(19) United States
(12) Patent Application Publication
(10) Pub. No.: US 2025/0268826 A1
(43) Pub. Date: Aug. 28, 2025

(54) IONIZABLE LIPIDOID COMPOSITIONS AND THERAPEUTIC USES THEREOF

(71) Applicant: Flagship Pioneering Innovations VII, LLC, Cambridge, MA (US)

(72) Inventors: Timothy Ray Blake, New York City, NY (US); Jae Hyeon Park, Belmont, MA (US); Dean Peter Stamos, Lexington, MA (US); Chelsea RamEI Martinez, Cambridge, MA (US)

(21) Appl. No.: 18/888,697

(22) Filed: Sep. 18, 2024

Related U.S. Application Data
(60) Provisional application No. 63/676,004, filed on Jul. 26, 2024.
(60) Provisional application No. 63/654,580, filed on May 31, 2024.
(60) Provisional application No. 63/557,884, filed on Feb. 26, 2024.
(60) Provisional application No. 63/538,946, filed on Sep. 18, 2023.

(51) Int. Cl. A61K 9/127; A61K 9/51; A61K 31/7105; A61K 39/00; A61K 39/215; C07C 237/08; C07C 237/20; C07D 207/06; C07D 211/12; C07D 233/64

(52) U.S. Cl. CPC A61K 9/1271; A61K 9/5123; A61K 31/7105; A61K 39/215; C07C 237/08; C07C 237/20; C07D 207/06; C07D 211/12; C07D 233/64; A61K 2039/53; A61K 2039/55555; C07C 2601/16

(57) ABSTRACT

Disclosed are lipidoid compounds having the structure of formula (X) or formula

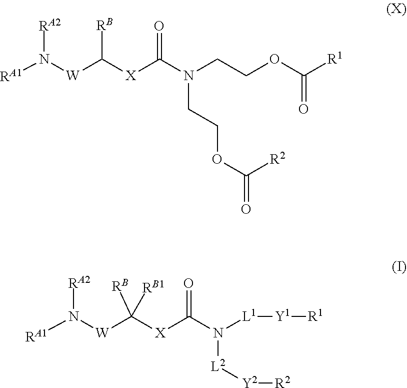

wherein the groups are as defined in the application. Also disclosed are nanoparticle compositions comprising a lipidoid of the invention that are capable of delivering a therapeutic agent. The application also discloses pharmaceutical compositions comprising a lipidoid composition of the invention.

### RELATED APPLICATIONS

This application claims the benefit of priority to U.S. Provisional Patent Application Ser. No. 63/538,946, filed Sep. 18, 2023; U.S. Provisional Patent Application Ser. No. 63/557,884, filed Feb. 26, 2024; U.S. Provisional Patent Application Ser. No. 63/654,580, filed May 31, 2024; and U.S. Provisional Patent Application Ser. No. 63/676,004, filed Jul. 26, 2024; the contents of each of which are incorporated herein by reference.

### BACKGROUND

The effective targeted delivery of biologically active substances such as small molecule drugs, proteins, and nucleic acids represents a continuing medical challenge. In particular, the delivery of nucleic acids to cells is made difficult by their relative instability and low cell permeability. Thus, there exists a need to develop methods and compositions to facilitate the delivery of therapeutic and/or prophylactics, such as nucleic acids, to cells.

Lipid-containing nanoparticle compositions, liposomes, and lipoplexes can be effective transport vehicles into cells and/or intracellular compartments for biologically active substances such as small molecule drugs, proteins, and nucleic acids. Such compositions generally include one or more cationic and/or ionizable lipids, phospholipids including polyunsaturated lipids, structural lipids (e.g., sterols), and/or lipids containing polyethylene glycol (PEG lipids). There is a need to develop lipidoids useful in lipid-nanoparticle compositions that can deliver therapeutic agents, such as nucleic acid molecules, proteins, and small molecule drugs with safety, efficacy, and specificity.

### SUMMARY

In certain aspects, provided here is a lipidoid having the structure of formula (X):

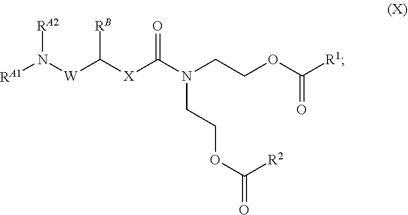

or a salt thereof;
wherein:
R^{A1} and R^{A2} are each independently H or alkyl;
   or wherein R^{A1} and R^{A2}, taken together with the intervening nitrogen, form an optionally substituted heterocyclic ring;
R^{B} is H or alkyl;
X is a bond or —CH₂—;
W is a bond, —CH₂—, or —CH₂CH₂—; and
R¹ and R² are each independently branched (C₈-C₃₀)alkyl or linear (C₈-C₃₀)alkenyl; wherein a branched (C₈-C₃₀)alkyl may comprise more than one branch point, and a linear (C₈-C₃₀)alkenyl may comprise more than one carbon-carbon double bond.

In further aspects, provided herein is a lipidoid having the structure of formula (X):

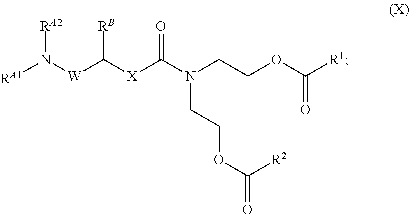

or a salt thereof;
wherein:
R^{A1} and R^{A2} are each alkyl;
   or wherein R^{A1} and R^{A2}, taken together with the intervening nitrogen, form an optionally substituted heterocyclic ring;
R^{B} is H or alkyl;
X is a bond or —CH₂—;
W is a bond, —CH₂—, or —CH₂CH₂—; and
R¹ and R² are each independently branched (C₈-C₃₀)alkyl; wherein a branched (C₈-C₃₀)alkyl may comprise more than one branch point.

In further aspects, provided here is a lipidoid having the structure of formula (I):

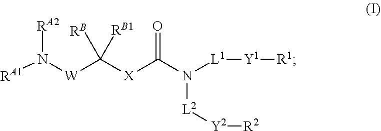

or a salt thereof;
wherein:
R^{A1} and R^{A2} are each independently H, alkyl, or hydroxyalkyl;
   or wherein R^{A1} and R^{A2}, taken together with the intervening nitrogen, form an optionally substituted heterocyclic ring;
R^{B} and R^{B1} are each independently H, optionally substituted alkyl, alkenyl, cycloalkyl, arylalkyl, heteroarylalkyl, hydroxyalkyl, alkoxyalkyl, aminoalkyl, or thioalkyl;
   or wherein R^{B} and R^{B1}, taken together with the intervening carbon, form an optionally substituted cycloalkyl or heterocyclic ring;
   or R^{A1} and R^{B} taken together with the intervening N—CH atoms to which they are attached form an N-containing heterocyclic ring;
L¹ and L² are each independently —CH₂CH₂—, —(CH₂)₃—, —(CH₂)₄—, —CH(CH₃)CH₂—, or —CH₂CH(CH₃)—;
X is a bond, —CH₂—, or —CH₂CH₂—;
W is a bond, —CH₂—, or —CH₂CH₂—;
Y¹ and Y² are each independently —O(C═O)—, —S(C═O)—, or —O(C═O)O—; and
R¹ and R² are each independently linear (C₈-C₃₀)alkyl, branched (C₈-C₃₀)alkyl, linear (C₈-C₃₀)alkenyl, branched (C₈-C₃₀)alkenyl, linear (C₈-C₃₀)alkynyl, or branched (C₈-C₃₀)alkynyl; wherein a branched (C₈-C₃₀)alkyl, a branched (C₈-C₃₀)alkenyl, or a branched (C₈-C₃₀)alkynyl may comprise more than one branch point, and a linear or branched (C₈-C₃₀)alkenyl may comprise more than one carbon-carbon double bond, and a linear or branched (C₈-C₃₀)alkynyl may comprise more than one carbon-carbon triple bond;
provided that the compound is not:
L-Lysine-bis(O,O′-oleoyl-β-hydroxyethyl)amide;
L-Lysine-bis(O,O′-palmitoyl-β-hydroxyethyl)amide;
L-Lysine-bis(O,O′-myristoyl-β-hydroxyethyl)amide;
L-Ornithine-bis(O,O′-palmitoyl-β-hydroxyethyl)amide;
L-Ornithine-bis(O,O′-oleoyl-β-hydroxyethyl)amide;
L-Ornithine-bis(O,O′-myristoyl-β-hydroxyethyl)amide;
L-Arginine-bis(O,O′-palmitoyl-β-hydroxyethyl)amide;
L-Arginine-bis(O,O′-oleoyl-β-hydroxyethyl)amide;
L-Serine-bis(O,O′-oleoyl-β-hydroxyethyl)amide;
Glycine-bis(O,O′-palmitoyl-β-hydroxyethyl)amide;
Sarcosine-bis(O,O′-palmitoyl-β-hydroxyethyl)amide;
L-Histidine-bis(O,O′-palmitoyl-β-hydroxyethyl)amide;
L-Glutamine-bis(O,O′-palmitoyl-β-hydroxyethyl)amide;
L-Glutamic acid-γ-N′-bis(O,O′-palmitoyl-β-hydroxyethyl)amide;
L-aspartic acid-β-N′-bis(O,O′-palmitoyl-β-hydroxyethyl)amide; or
L-aspartic acid-α-N′-bis(O,O′-palmitoyl-β-hydroxyethyl)amide.

In further aspects, provided here is a lipidoid having the structure of formula (II):

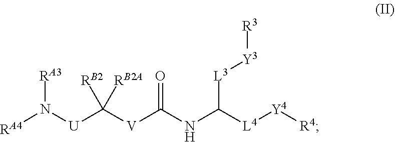

or a salt thereof;
wherein:
R^{A3} and R^{A4} are each independently H, alkyl, or hydroxyalkyl;
   or wherein R^{A3} and R^{A4}, taken together with the intervening nitrogen, form an optionally substituted heterocycloalkyl ring;
R^{B2} and R^{B2A} are each independently H, optionally substituted alkyl, alkenyl, cycloalkyl, arylalkyl, heteroarylalkyl, hydroxyalkyl, alkoxyalkyl, aminoalkyl, or thioalkyl;
   or wherein R^{B2} and R^{B2A}, taken together with the intervening carbon, form an optionally substituted cycloalkyl or heterocycloalkyl ring;
   or R^{A3} and R^{B2} taken together with the intervening N—CH atoms to which they are attached form an N-containing heterocyclic ring;
L³ and L⁴ are each independently —CH₂—, —CH₂CH₂—, —(CH₂)₃—, —(CH₂)₄—, —CH(CH₃)CH₂—, or —CH₂CH(CH₃)—;
V is a bond, —CH₂— or —CH₂CH₂—;
U is a bond, —CH₂— or —CH₂CH₂—;
Y³ and Y⁴ are each independently —O(C═O)—, —S(C═O)—, or —O(C═O)O—; and
R³ and R⁴ are each independently linear (C₈-C₃₀)alkyl, branched (C₈-C₃₀)alkyl, linear (C₈-C₃₀)alkenyl, branched (C₈-C₃₀)alkenyl, linear (C₈-C₃₀)alkynyl, or branched (C₈-C₃₀)alkynyl; wherein a branched (C₈-C₃₀)alkyl, a branched (C₈-C₃₀)alkenyl, or a branched (C₈-C₃₀)alkynyl may comprise more than one branch point, and a linear or branched (C₈-C₃₀)alkenyl may comprise more than one carbon-carbon double bond, and a linear or branched (C₈-C₃₀)alkynyl may comprise more than one carbon-carbon triple bond.

In certain embodiments, the invention provides nanoparticle compositions comprising a lipidoid of the invention.

The invention also provides methods of delivering a therapeutic agent, comprising administering to a subject in need thereof an effective amount of a nanoparticle composition of the invention, wherein the nanoparticle composition comprises a therapeutic agent.

The invention also provides methods of vaccination, comprising administering to a subject in need thereof an effective amount of a nanoparticle composition of the invention, wherein the nanoparticle composition comprises an antigen.

Also provided herein are pharmaceutical compositions, comprising a nanoparticle composition of the invention, and a pharmaceutically acceptable carrier.

### BRIEF DESCRIPTION OF THE DRAWINGS

FIG. 1 shows the in vivo LNP potency assessment by readout of hEPO mRNA expression for Series 100 lipids of the invention.

FIG. 2 shows the in vitro LNP potency assessment by cellular EGFP mRNA expression with compositional modifications on Series 100 and Series 300 lipids.

FIG. 3 shows the in vivo LNP potency assessment by readout of hEPO mRNA expression for Series 100, 200, and 300 lipids.

FIG. 4 shows the in vivo LNP potency assessment by readout of hEPO mRNA expression Compound 117 with compositional optimization.

FIG. 5 shows the in vivo LNP potency assessment by readout of hEPO mRNA expression for Series 600 LNPs with or without compositional optimization.

FIG. 6 shows the in vivo LNP potency assessment by readout of hEPO mRNA expression for Series 800 LNPs with or without compositional optimization.

FIG. 7 shows the in vivo LNP potency assessment of hEPO mRNA expression of Compound 808 with compositional modifications.

FIG. 8 shows the in vivo LNP potency assessment by readout of hEPO mRNA expression for Series 900 LNPs.

FIG. 9 shows the in vivo LNP potency assessment by readout of hEPO mRNA expression for Series 900 LNPs.

FIG. 10 shows the in vivo LNP potency assessment by readout of hEPO mRNA expression for Series 900 LNPs.

FIG. 11 shows the in vivo LNP potency assessment by readout of hEPO mRNA expression for Series 900 LNPs.

FIG. 12 shows the antibody titers in mice 28 days after administration of an LNP loaded with mRNA encoding SARS-CoV-2 spike proteins.

FIG. 13 depicts bar graphs showing the size and encapsulation efficiency of LNPs loaded with mRNA encoding SARS-CoV-2 spike proteins.

FIG. 14 shows the antibody titers in mice 28 days after administration of an LNP loaded with mRNA encoding SARS-CoV-2 spike proteins.

FIG. 15 depicts bar graphs showing the size and encapsulation efficiency of LNPs loaded with mRNA encoding SARS-CoV-2 spike proteins.

FIG. 16 depicts bar graphs showing the zeta potential and apparent pKₐ of LNPs loaded with mRNA encoding SARS-CoV-2 spike proteins.

FIG. 17 shows the in vivo LNP potency assessment by readout of hEPO mRNA expression for Series 900 LNPs.

### DETAILED DESCRIPTION

The present invention is based on the surprising discovery of a class of lipidoid compounds useful for forming lipid nanoparticles that can deliver therapeutic agents.

In certain embodiments, provided herein is a lipidoid having the structure of formula

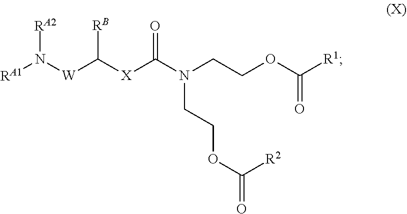

or a salt thereof;
wherein:
R^{A1} and R^{A2} are each independently H or alkyl;
or wherein R^{A1} and R^{A2}, taken together with the intervening nitrogen, form an optionally substituted heterocyclic ring;
R^{B} is H or alkyl;
X is a bond or —CH₂—;
W is a bond, —CH₂—, or —CH₂CH₂—; and
R¹ and R² are each independently branched (C₈-C₃₀)alkyl or linear (C₈-C₃₀)alkenyl; wherein a branched (C₈-C₃₀)alkyl may comprise more than one branch point, and a linear (C₈-C₃₀)alkenyl may comprise more than one carbon-carbon double bond.

In certain embodiments, R^{A1} and R^{A2} are the same.

In alternative embodiments, R^{A1} and R^{A2} are different.

In certain embodiments, R^{A1} and R^{A2} are each independently selected from H, methyl, ethyl, propyl, and isobutyl.

In certain other embodiments, R^{A1} and R^{A2}, taken together with the intervening nitrogen, form an optionally substituted 5-membered or 6-membered heterocyclic ring.

In certain embodiments, R^{B} is alkyl.

In other embodiments, R^{B} is methyl.

In certain embodiments, R¹ and R² are the same.

In certain embodiments, R¹ and R² are different.

In certain embodiments, R¹ and R² are each independently branched (C₁₂-C₂₀)alkyl.

In certain embodiments, if R^{B} is H and R^{A1} and R^{A2} are both methyl, then R¹ and R² are each independently branched (C₈-C₃₀)alkyl.

In certain preferred embodiments, the lipidoid has the structure of formula (X):

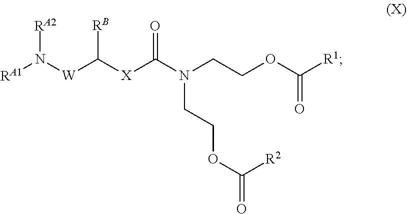

or a salt thereof;
wherein:
R^{A1} and R^{A2} are each alkyl;
   or wherein R^{A1} and R^{A2}, taken together with the intervening nitrogen, form an optionally substituted heterocyclic ring;
R^{B} is H or alkyl;
X is a bond or —CH₂—;
W is a bond, —CH₂—, or —CH₂CH₂—; and
R¹ and R² are each independently branched (C₈-C₃₀)alkyl; wherein a branched (C₈-C₃₀)alkyl may comprise more than one branch point.

In certain embodiments, R^{A1} and R^{A2} are the same.

In certain embodiments, R^{A1} and R^{A2} are different.

In certain embodiments, R^{A1} and R^{A2} are each independently selected from methyl, ethyl, propyl, and isobutyl.

In certain embodiments, R^{A1} and R^{A2}, taken together with the intervening nitrogen, form an optionally substituted 5-membered or 6-membered heterocyclic ring.

In some embodiments, R^{B} is alkyl.

In certain preferred embodiments, R^{B} is methyl.

In some embodiments, R¹ and R² are the same.

In certain other embodiments, R¹ and R² are different.

In certain embodiments, R¹ and R² are each independently branched (C₁₂-C₂₀)alkyl.

In certain embodiments, when X is a bond, then W is —CH₂— or —CH₂CH₂—; and when W is a bond, then X is —CH₂—.

In certain embodiments, when X is a bond, then W is —CH₂—; and when W is a bond, then X is —CH₂—.

In certain embodiments, the present disclosure provides a lipidoid having the structure of formula (I):

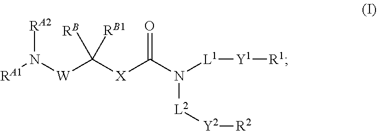

or a salt thereof;
wherein:
R^{A1} and R^{A2} are each independently H, alkyl, or hydroxyalkyl;
   or wherein R^{A1} and R^{A2}, taken together with the intervening nitrogen, form an optionally substituted heterocyclic ring;
R^{B} and R^{B1} are each independently H, optionally substituted alkyl, alkenyl, cycloalkyl, arylalkyl, heteroarylalkyl, hydroxyalkyl, alkoxyalkyl, aminoalkyl, or thioalkyl;
   or wherein R^{B} and R^{B1}, taken together with the intervening carbon, form an optionally substituted cycloalkyl or heterocyclic ring;
   or R^{A1} and R^{B} taken together with the intervening N—CH atoms to which they are attached form an N-containing heterocyclic ring;
L¹ and L² are each independently —CH₂CH₂—, —(CH₂)₃—, —(CH₂)₄—, —CH(CH₃)CH₂—, or —CH₂CH(CH₃)—;
X is a bond, —CH₂—, or —CH₂CH₂—;
W is a bond, —CH₂—, or —CH₂CH₂—;
Y¹ and Y² are each independently —O(C═O)—, —S(C═O)—, or —O(C═O)O—; and
R¹ and R² are each independently linear (C₈-C₃₀)alkyl, branched (C₈-C₃₀)alkyl, linear (C₈-C₃₀)alkenyl, branched (C₈-C₃₀)alkenyl, linear (C₈-C₃₀)alkynyl, or branched (C₈-C₃₀)alkynyl; wherein a branched (C₈-C₃₀)alkyl, a branched (C₈-C₃₀)alkenyl, or a branched (C₈-C₃₀)alkynyl may comprise more than one branch point, and a linear or branched (C₈-C₃₀)alkenyl may comprise more than one carbon-carbon double bond, and a linear or branched (C₈-C₃₀)alkynyl may comprise more than one carbon-carbon triple bond;
provided that the compound is not:
L-Lysine-bis(O,O′-oleoyl-β-hydroxyethyl)amide;
L-Lysine-bis(O,O′-palmitoyl-β-hydroxyethyl)amide;
L-Lysine-bis(O,O′-myristoyl-β-hydroxyethyl)amide;
L-Ornithine-bis(O,O′-palmitoyl-β-hydroxyethyl)amide;
L-Ornithine-bis(O,O′-oleoyl-β-hydroxyethyl)amide;
L-Ornithine-bis(O,O′-myristoyl-β-hydroxyethyl)amide;
L-Arginine-bis(O,O′-palmitoyl-β-hydroxyethyl)amide;
L-Arginine-bis(O,O′-oleoyl-β-hydroxyethyl)amide;
L-Serine-bis(O,O′-oleoyl-β-hydroxyethyl)amide;
Glycine-bis(O,O′-palmitoyl-β-hydroxyethyl)amide;
Sarcosine-bis(O,O′-palmitoyl-β-hydroxyethyl)amide;
L-Histidine-bis(O,O′-palmitoyl-β-hydroxyethyl)amide;
L-Glutamine-bis(O,O′-palmitoyl-β-hydroxyethyl)amide;
L-Glutamic acid-γ-N′-bis(O,O′-palmitoyl-β-hydroxyethyl)amide;
L-aspartic acid-β-N′-bis(O,O′-palmitoyl-β-hydroxyethyl)amide; or
L-aspartic acid-α-N′-bis(O,O′-palmitoyl-β-hydroxyethyl)amide.

In certain embodiments, when R^{B} and R^{B1} are both H and R^{A1} and R^{A2} are both methyl, then R¹ and R² are each independently branched (C₈-C₃₀)alkyl.

In certain embodiments,

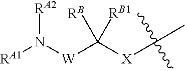

is represented by

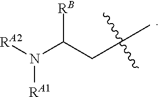

In further embodiments,

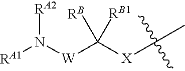

is represented by

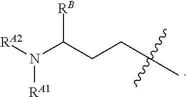

In yet further embodiments,

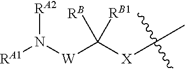

is represented by

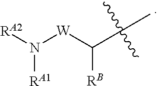

In certain embodiments, R^{A1} and R^{A2} are the same.

In certain preferred embodiments, R^{A1} and R^{A2} are each H.

In yet other embodiments, R^{A1} and R^{A2} are different.

In certain embodiments, R^{A1} and R^{A2} are each independently selected from H, methyl, ethyl, propyl, and isobutyl.

In some embodiments, R^{A1} and R^{A2}, taken together with the intervening nitrogen, form an optionally substituted 5-membered or 6-membered heterocyclic ring.

In certain other embodiments, R^{A1} is hydroxyalkyl; and R^{A2} is H.

In certain embodiments, R^{A1} is —CH₂CH₂OH.

In yet other embodiments, R^{B} is H, optionally substituted alkyl, arylalkyl, heteroarylalkyl, hydroxyalkyl, or aminoalkyl.

In certain embodiments, R^{B} is H, methyl, —CH₂Ph, —CH₂OH, —(CH₂)₄NH₂, or —CH₂(imidazolyl).

In certain embodiments, R^{B} is methyl.

In some embodiments, R^{B1} is hydrogen.

In certain embodiments, R^{B} or R^{B1} is alkyl.

In yet other embodiments, R^{B} or R^{B1} is methyl.

In certain preferred embodiments, at least one of X and W is a bond.

In alternative embodiments, X and W are both a bond.

In certain embodiments, the disclosure provides a lipidoid having the structure of formula (La):

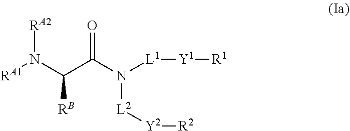

or a salt thereof;
wherein the lipidoid has the absolute stereochemistry shown.

In certain embodiments, the disclosure provides a lipidoid having the structure of formula (Ib):

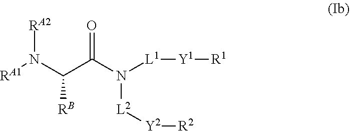

or a salt thereof;
wherein the lipidoid has the absolute stereochemistry shown.

In certain embodiments, X is —CH₂—; and W is a bond.

In certain embodiments, the disclosure provides a lipidoid having the structure of formula (Ic):

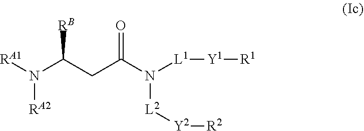

or a salt thereof;
wherein the lipidoid has the absolute stereochemistry shown.

In certain embodiments, the disclosure provides a lipidoid having the structure of formula (Id):

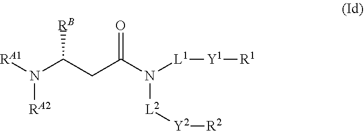

or a salt thereof;
wherein the lipidoid has the absolute stereochemistry shown.

In certain embodiments, X is —CH₂CH₂—; and W is a bond.

In certain embodiments, the disclosure provides a lipidoid having the structure of formula (Ie):

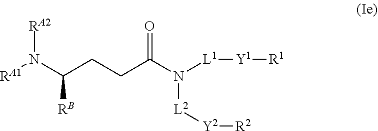

or a salt thereof;
wherein the lipidoid has the absolute stereochemistry shown.

In yet other embodiments, the disclosure provides a lipidoid having the structure of formula (If):

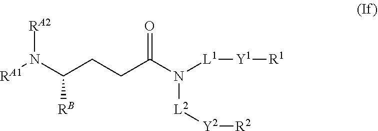

or a salt thereof;
wherein the lipidoid has the absolute stereochemistry shown.

In certain embodiments, W is a bond.

In yet other embodiments, Y¹ and Y² are each —O(C═O)—

In some embodiments, L¹ and L² each represent —CH₂CH₂—.

In further embodiments, R¹ and R² are the same

In certain embodiments, R¹ and R² are different.

In certain other embodiments, R¹ and R² are each independently linear (C₈-C₂₄)alkyl, branched (C₈-C₂₄)alkyl, linear (C₈-C₂₄)alkenyl, or branched (C₈-C₂₄)alkenyl.

In some preferred embodiments, R¹ and R² are each independently linear (C₈-C₂₄)alkyl or linear (C₈-C₂₄)alkenyl.

In some embodiments, R¹ and R² are each independently linear (C₈-C₂₄)alkyl.

In certain embodiments, R¹ and R² are each independently linear (C₉-C₁₇)alkyl.

In alternative embodiments, R¹ and R² are each independently linear (C₈-C₂₄)alkenyl.

In certain preferred embodiments, R¹ and R² are each independently a linear (C₁₇)alkenyl.

In certain embodiments, R¹ and R² are each independently branched (C₈-C₂₄)alkyl.

In certain embodiments, R¹ and R² are each independently branched (C₁₂-C₂₀)alkyl.

In further embodiments, R¹ and R² are each independently a branched (C₁₅-C₁₉)alkyl.

In certain embodiments, the present disclosure provides a lipidoid having the structure of formula (Ig):

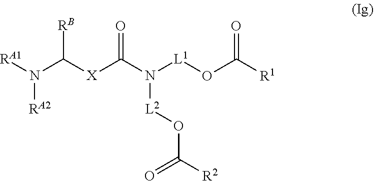

or a salt thereof.

In certain embodiments, R^{B} is aminoalkyl, R^{B1} is hydrogen, X and W are both a bond, R^{A1} and R^{A2} are each H, L¹ and L² are each —CH₂CH₂—; and R¹ and R² are each independently branched (C₈-C₃₀)alkyl.

In certain embodiments, R^{B} is hydroxyalkyl, R^{B1} is hydrogen, X and W are both a bond, R^{A1} and R^{A2} are each H, L¹ and L² are each —CH₂CH₂—; and R¹ and R² are each independently linear or branched (C₈-C₃₀)alkyl.

In certain embodiments, R^{B} is H, R^{Bl} is hydrogen, X and W are both a bond, R^{A1} is H, R^{A2} is H or methyl, L¹ and L² are each —CH₂CH₂—; and R¹ and R² are each independently branched (C₈-C₃₀)alkyl, linear (C₈-C₃₀)alkenyl, or branched (C₈-C₃₀)alkenyl.

In certain embodiments, R^{B} is heteroarylalkyl, R^{B1} is hydrogen, X and W are both a bond, R^{A1} and R^{A2} are each H, L¹ and L² are each —CH₂CH₂—; and R¹ and R² are each independently branched (C₈-C₃₀)alkyl, linear (C₈-C₃₀)alkenyl, or branched (C₈-C₃₀)alkenyl.

In any of the preceding embodiments, Y¹ and Y² can each be —O(C═O)—.

Representative Series 100 lipidoids include those in the table below.

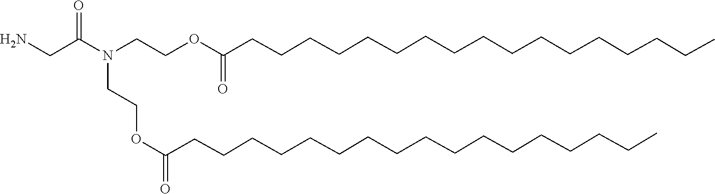 [SMILES: CCCCCCCCCCCCCCCCCC(=O)OCCN(CCOC(=O)CCCCCCCCCCCCCCCCC)C(=O)CN]

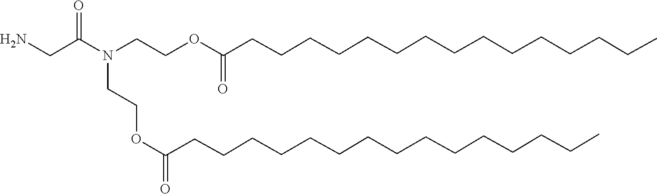 [SMILES: CCCCCCCCCCCCCCCC(=O)OCCN(CCOC(=O)CCCCCCCCCCCCCCC)C(=O)CN]

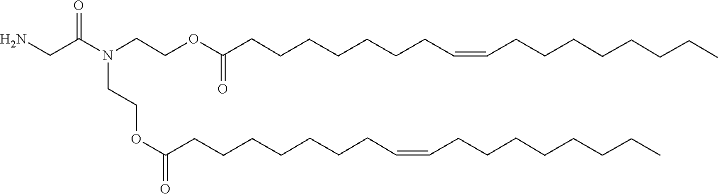 [SMILES: CCCCCCCC/C=C\CCCCCCCC(=O)OCCN(CCOC(=O)CCCCCCC/C=C\CCCCCCCC)C(=O)CN]

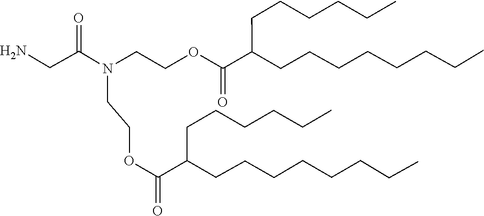 [SMILES: CCCCCCCCC(CCCCCC)C(=O)OCCN(CCOC(=O)C(CCCCCC)CCCCCCCC)C(=O)CN]

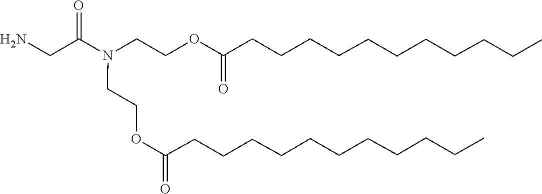 [SMILES: CCCCCCCCCCCC(=O)OCCN(CCOC(=O)CCCCCCCCCCC)C(=O)CN]

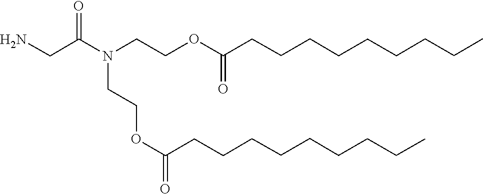 [SMILES: CCCCCCCCCC(=O)OCCN(CCOC(=O)CCCCCCCCC)C(=O)CN]

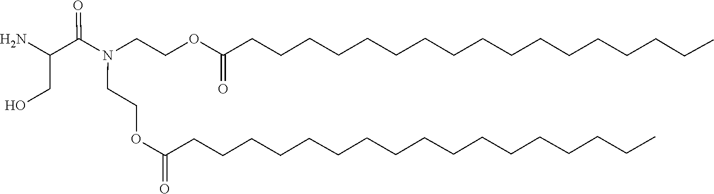 [SMILES: CCCCCCCCCCCCCCCCCC(=O)OCCN(CCOC(=O)CCCCCCCCCCCCCCCCC)C(=O)C(N)CO]

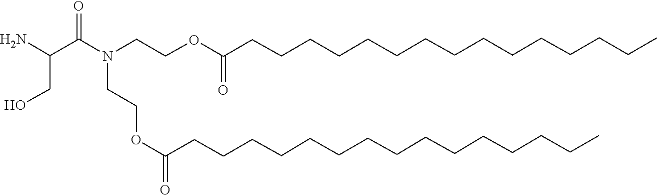 [SMILES: CCCCCCCCCCCCCCCC(=O)OCCN(CCOC(=O)CCCCCCCCCCCCCCC)C(=O)C(N)CO]

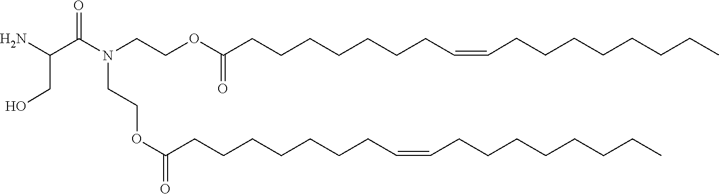 [SMILES: CCCCCCCC/C=C\CCCCCCCC(=O)OCCN(CCOC(=O)CCCCCCC/C=C\CCCCCCCC)C(=O)C(N)CO]

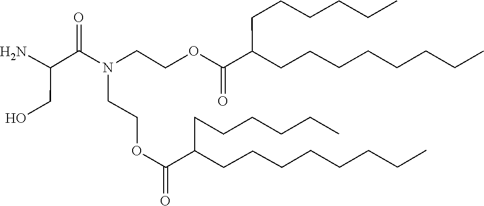 [SMILES: CCCCCCCCC(CCCCCC)C(=O)OCCN(CCOC(=O)C(CCCCCC)CCCCCCCC)C(=O)C(N)CO]

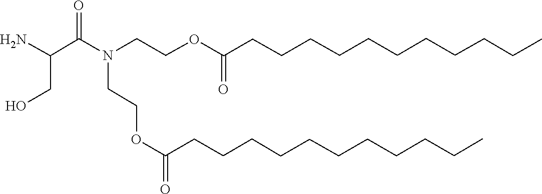 [SMILES: CCCCCCCCCCCC(=O)OCCN(CCOC(=O)CCCCCCCCCCC)C(=O)C(N)CO]

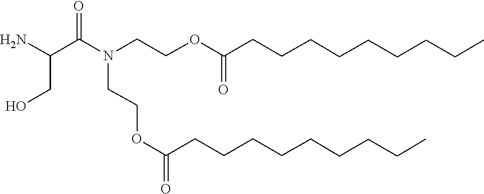 [SMILES: CCCCCCCCCC(=O)OCCN(CCOC(=O)CCCCCCCCC)C(=O)C(N)CO]

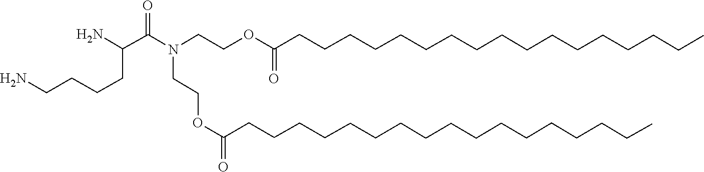 [SMILES: CCCCCCCCCCCCCCCCCC(=O)OCCN(CCOC(=O)CCCCCCCCCCCCCCCCC)C(=O)C(N)CCCCN]

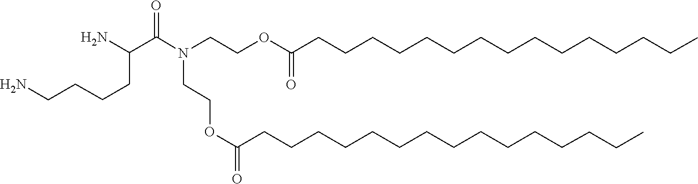 [SMILES: CCCCCCCCCCCCCCCC(=O)OCCN(CCOC(=O)CCCCCCCCCCCCCCC)C(=O)C(N)CCCCN]

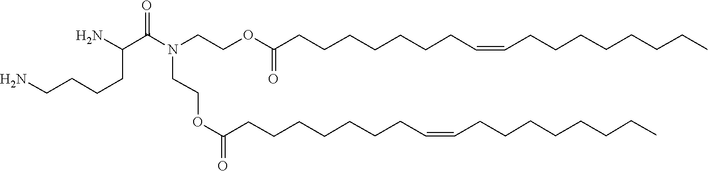 [SMILES: CCCCCCCC/C=C\CCCCCCCC(=O)OCCN(CCOC(=O)CCCCCCC/C=C\CCCCCCCC)C(=O)C(N)CCCCN]

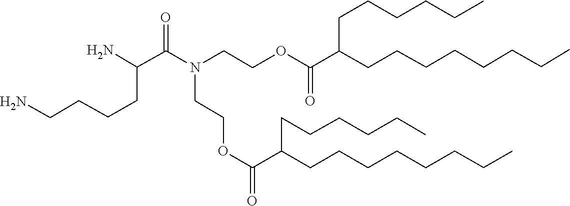 [SMILES: CCCCCCCCC(CCCCCC)C(=O)OCCN(CCOC(=O)C(CCCCCC)CCCCCCCC)C(=O)C(N)CCCCN]

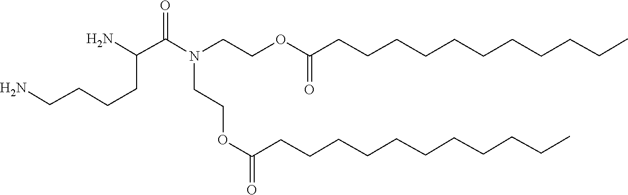 [SMILES: CCCCCCCCCCCC(=O)OCCN(CCOC(=O)CCCCCCCCCCC)C(=O)C(N)CCCCN]

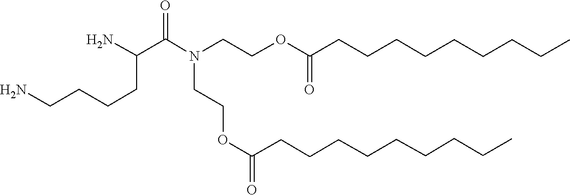 [SMILES: CCCCCCCCCC(=O)OCCN(CCOC(=O)CCCCCCCCC)C(=O)C(N)CCCCN]

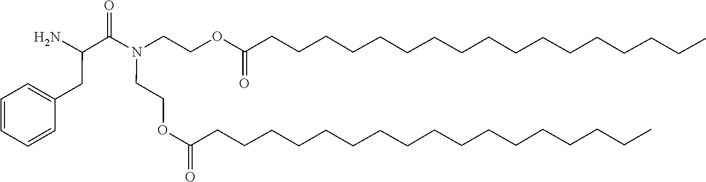 [SMILES: CCCCCCCCCCCCCCCCCC(=O)OCCN(CCOC(=O)CCCCCCCCCCCCCCCCC)C(=O)C(N)Cc1ccccc1]

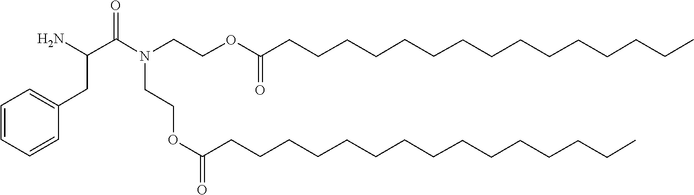 [SMILES: CCCCCCCCCCCCCCCC(=O)OCCN(CCOC(=O)CCCCCCCCCCCCCCC)C(=O)C(N)Cc1ccccc1]

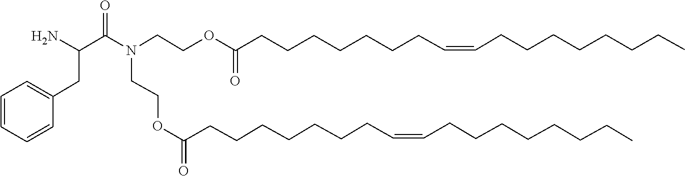 [SMILES: CCCCCCCC/C=C\CCCCCCCC(=O)OCCN(CCOC(=O)CCCCCCC/C=C\CCCCCCCC)C(=O)C(N)Cc1ccccc1]

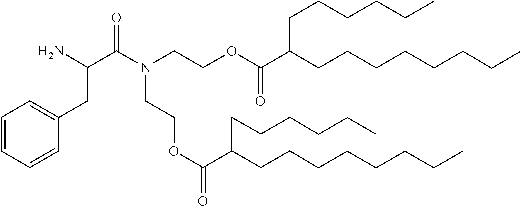 [SMILES: CCCCCCCCC(CCCCCC)C(=O)OCCN(CCOC(=O)C(CCCCCC)CCCCCCCC)C(=O)C(N)Cc1ccccc1]

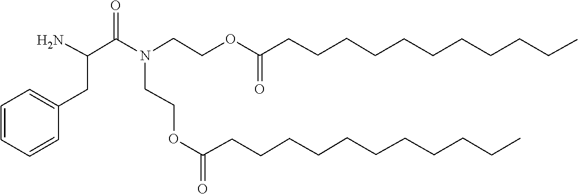 [SMILES: CCCCCCCCCCCC(=O)OCCN(CCOC(=O)CCCCCCCCCCC)C(=O)C(N)Cc1ccccc1]

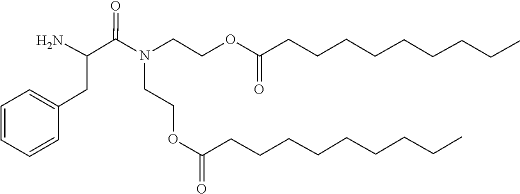 [SMILES: CCCCCCCCCC(=O)OCCN(CCOC(=O)CCCCCCCCC)C(=O)C(N)Cc1ccccc1]

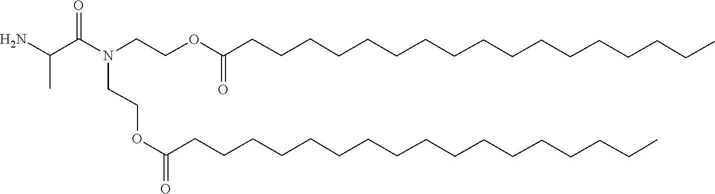 [SMILES: CCCCCCCCCCCCCCCCCC(=O)OCCN(CCOC(=O)CCCCCCCCCCCCCCCCC)C(=O)C(C)N]

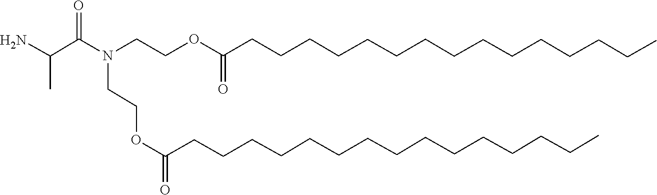 [SMILES: CCCCCCCCCCCCCCCC(=O)OCCN(CCOC(=O)CCCCCCCCCCCCCCC)C(=O)C(C)N]

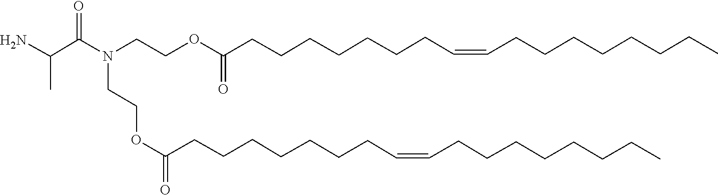 [SMILES: CCCCCCCC/C=C\CCCCCCCC(=O)OCCN(CCOC(=O)CCCCCCC/C=C\CCCCCCCC)C(=O)C(C)N]

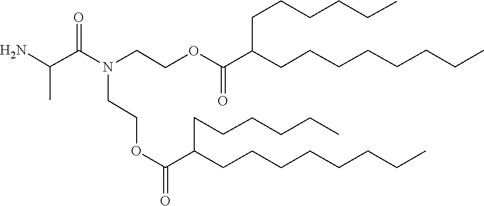 [SMILES: CCCCCCCCC(CCCCCC)C(=O)OCCN(CCOC(=O)C(CCCCCC)CCCCCCCC)C(=O)C(C)N]

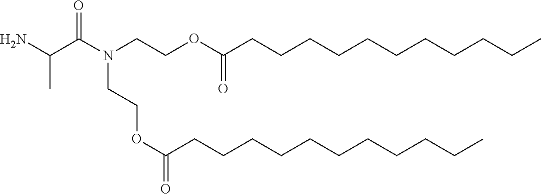 [SMILES: CCCCCCCCCCCC(=O)OCCN(CCOC(=O)CCCCCCCCCCC)C(=O)C(C)N]

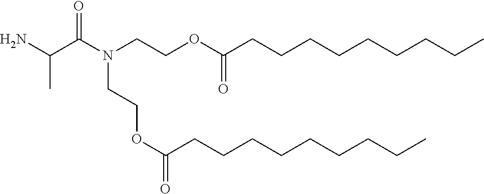 [SMILES: CCCCCCCCCC(=O)OCCN(CCOC(=O)CCCCCCCCC)C(=O)C(C)N]

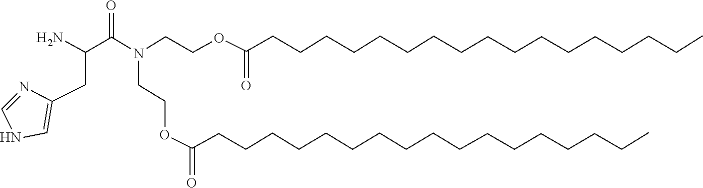 [SMILES: CCCCCCCCCCCCCCCCCC(=O)OCCN(CCOC(=O)CCCCCCCCCCCCCCCCC)C(=O)C(N)Cc1c[nH]cn1]

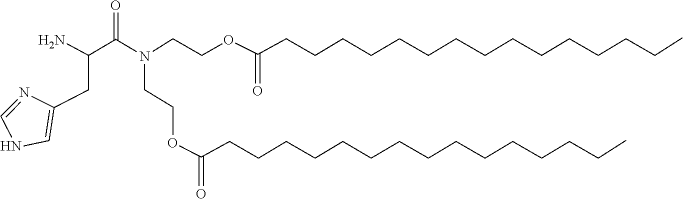 [SMILES: CCCCCCCCCCCCCCCC(=O)OCCN(CCOC(=O)CCCCCCCCCCCCCCC)C(=O)C(N)Cc1c[nH]cn1]

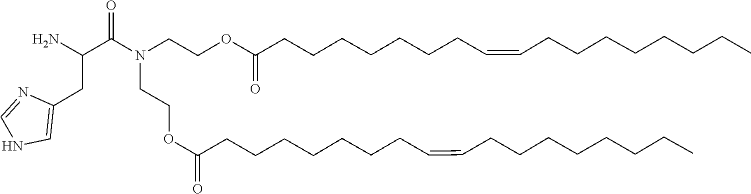 [SMILES: CCCCCCCC/C=C\CCCCCCCC(=O)OCCN(CCOC(=O)CCCCCCC/C=C\CCCCCCCC)C(=O)C(N)Cc1c[nH]cn1]

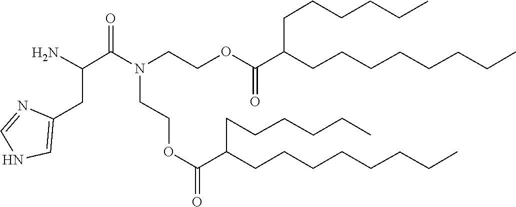 [SMILES: CCCCCCCCC(CCCCCC)C(=O)OCCN(CCOC(=O)C(CCCCCC)CCCCCCCC)C(=O)C(N)Cc1c[nH]cn1]

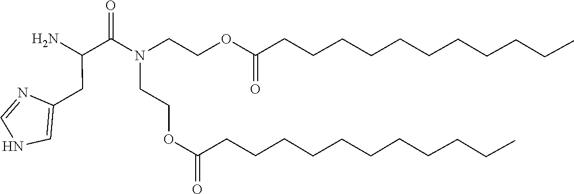 [SMILES: CCCCCCCCCCCC(=O)OCCN(CCOC(=O)CCCCCCCCCCC)C(=O)C(N)Cc1c[nH]cn1]

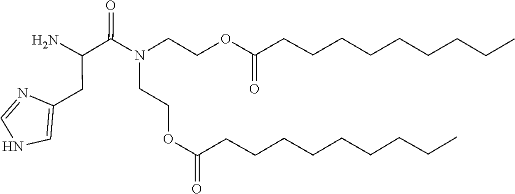 [SMILES: CCCCCCCCCC(=O)OCCN(CCOC(=O)CCCCCCCCC)C(=O)C(N)Cc1c[nH]cn1]

Representative Series 300 lipidoids include those in the table below:

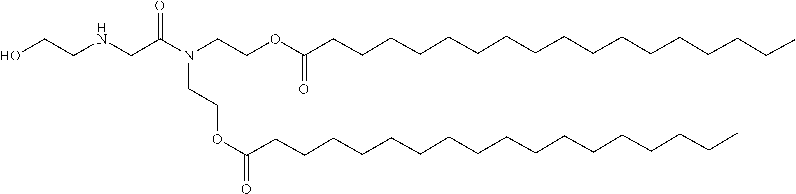 [SMILES: CCCCCCCCCCCCCCCCCC(=O)OCCN(CCOC(=O)CCCCCCCCCCCCCCCCC)C(=O)CNCCO]

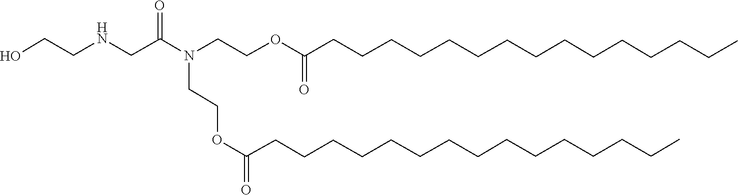 [SMILES: CCCCCCCCCCCCCCCC(=O)OCCN(CCOC(=O)CCCCCCCCCCCCCCC)C(=O)CNCCO]

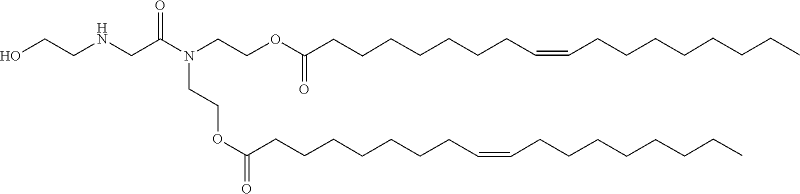 [SMILES: CCCCCCCC/C=C\CCCCCCCC(=O)OCCN(CCOC(=O)CCCCCCC/C=C\CCCCCCCC)C(=O)CNCCO]

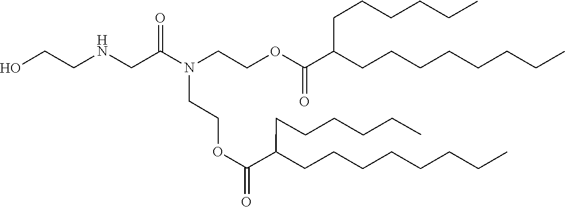 [SMILES: CCCCCCCCC(CCCCCC)C(=O)OCCN(CCOC(=O)C(CCCCCC)CCCCCCCC)C(=O)CNCCO]

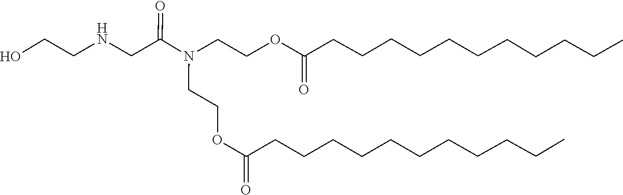 [SMILES: CCCCCCCCCCCC(=O)OCCN(CCOC(=O)CCCCCCCCCCC)C(=O)CNCCO]

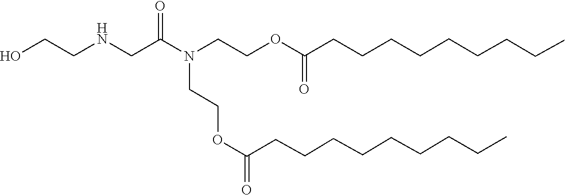 [SMILES: CCCCCCCCCC(=O)OCCN(CCOC(=O)CCCCCCCCC)C(=O)CNCCO]

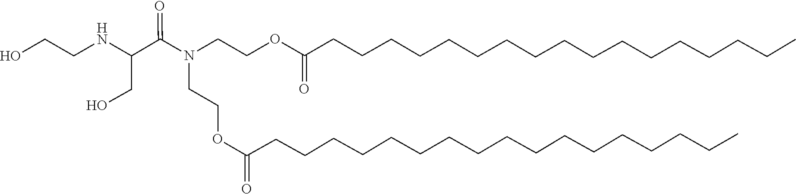 [SMILES: CCCCCCCCCCCCCCCCCC(=O)OCCN(CCOC(=O)CCCCCCCCCCCCCCCCC)C(=O)C(CO)NCCO]

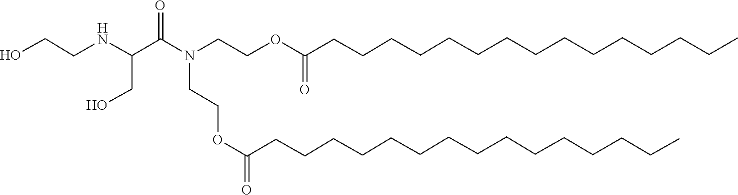 [SMILES: CCCCCCCCCCCCCCCC(=O)OCCN(CCOC(=O)CCCCCCCCCCCCCCC)C(=O)C(CO)NCCO]

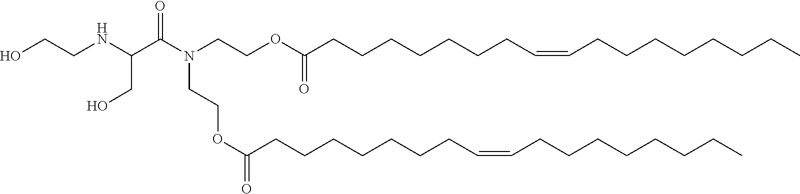 [SMILES: CCCCCCCC/C=C\CCCCCCCC(=O)OCCN(CCOC(=O)CCCCCCC/C=C\CCCCCCCC)C(=O)C(CO)NCCO]

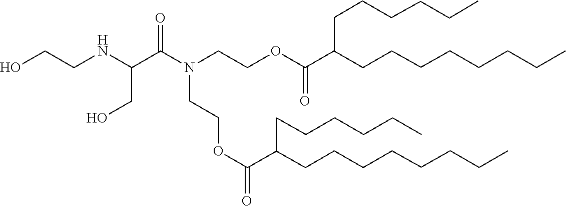 [SMILES: CCCCCCCCC(CCCCCC)C(=O)OCCN(CCOC(=O)C(CCCCCC)CCCCCCCC)C(=O)C(CO)NCCO]

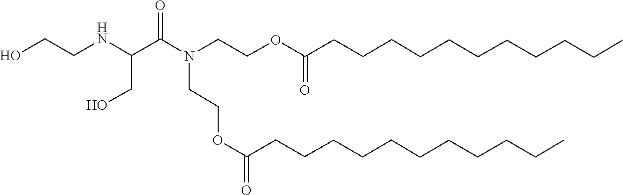 [SMILES: CCCCCCCCCCCC(=O)OCCN(CCOC(=O)CCCCCCCCCCC)C(=O)C(CO)NCCO]

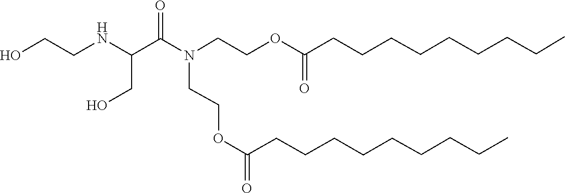 [SMILES: CCCCCCCCCC(=O)OCCN(CCOC(=O)CCCCCCCCC)C(=O)C(CO)NCCO]

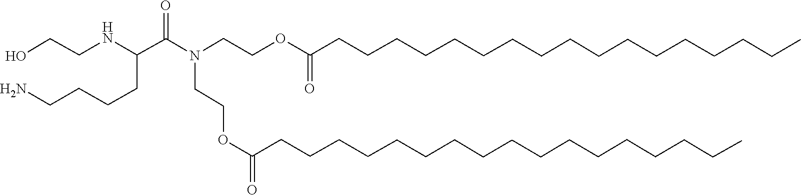 [SMILES: CCCCCCCCCCCCCCCCCC(=O)OCCN(CCOC(=O)CCCCCCCCCCCCCCCCC)C(=O)C(CCCCN)NCCO]

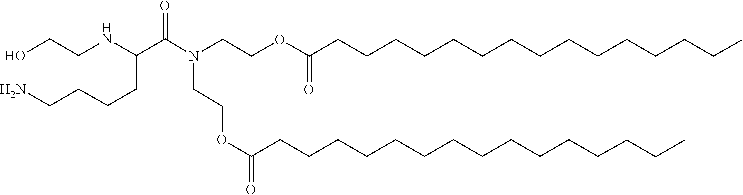 [SMILES: CCCCCCCCCCCCCCCC(=O)OCCN(CCOC(=O)CCCCCCCCCCCCCCC)C(=O)C(CCCCN)NCCO]

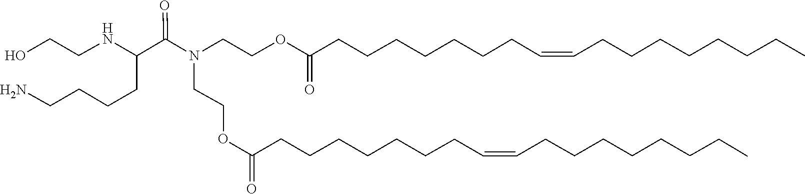 [SMILES: CCCCCCCC/C=C\CCCCCCCC(=O)OCCN(CCOC(=O)CCCCCCC/C=C\CCCCCCCC)C(=O)C(CCCCN)NCCO]

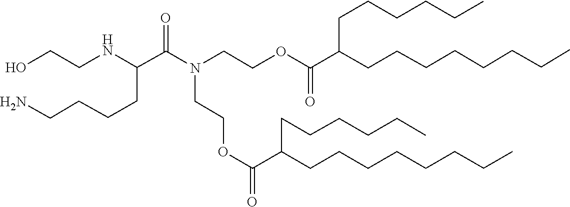 [SMILES: CCCCCCCCC(CCCCCC)C(=O)OCCN(CCOC(=O)C(CCCCCC)CCCCCCCC)C(=O)C(CCCCN)NCCO]

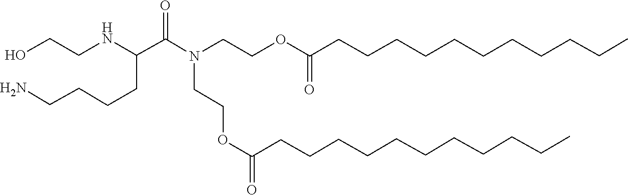 [SMILES: CCCCCCCCCCCC(=O)OCCN(CCOC(=O)CCCCCCCCCCC)C(=O)C(CCCCN)NCCO]

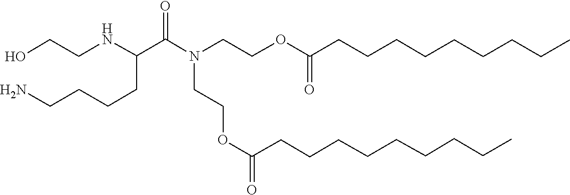 [SMILES: CCCCCCCCCC(=O)OCCN(CCOC(=O)CCCCCCCCC)C(=O)C(CCCCN)NCCO]

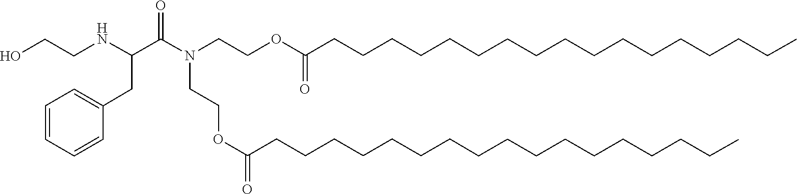 [SMILES: CCCCCCCCCCCCCCCCCC(=O)OCCN(CCOC(=O)CCCCCCCCCCCCCCCCC)C(=O)C(Cc1ccccc1)NCCO]

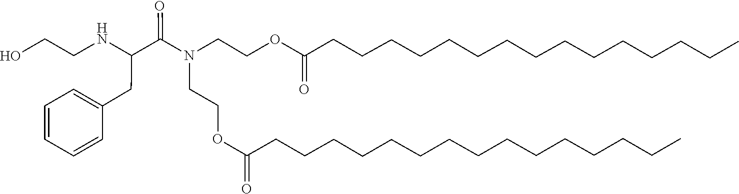 [SMILES: CCCCCCCCCCCCCCCC(=O)OCCN(CCOC(=O)CCCCCCCCCCCCCCC)C(=O)C(Cc1ccccc1)NCCO]

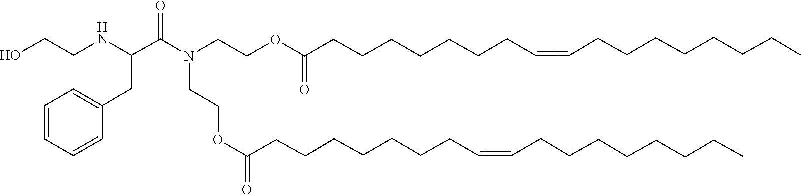 [SMILES: CCCCCCCC/C=C\CCCCCCCC(=O)OCCN(CCOC(=O)CCCCCCC/C=C\CCCCCCCC)C(=O)C(Cc1ccccc1)NCCO]

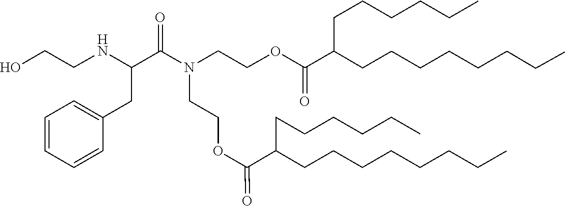 [SMILES: CCCCCCCCC(CCCCCC)C(=O)OCCN(CCOC(=O)C(CCCCCC)CCCCCCCC)C(=O)C(Cc1ccccc1)NCCO]

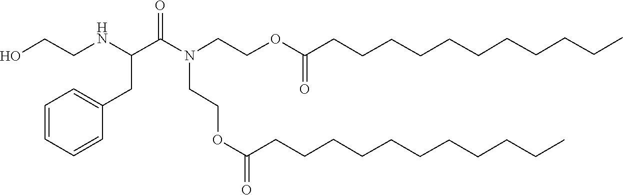 [SMILES: CCCCCCCCCCCC(=O)OCCN(CCOC(=O)CCCCCCCCCCC)C(=O)C(Cc1ccccc1)NCCO]

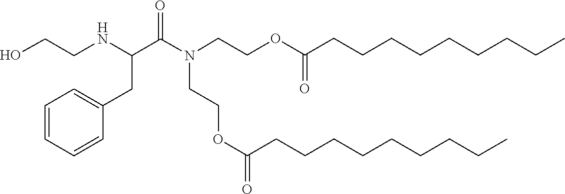 [SMILES: CCCCCCCCCC(=O)OCCN(CCOC(=O)CCCCCCCCC)C(=O)C(Cc1ccccc1)NCCO]

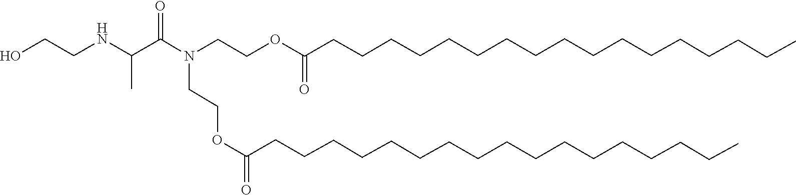 [SMILES: CCCCCCCCCCCCCCCCCC(=O)OCCN(CCOC(=O)CCCCCCCCCCCCCCCCC)C(=O)C(C)NCCO]

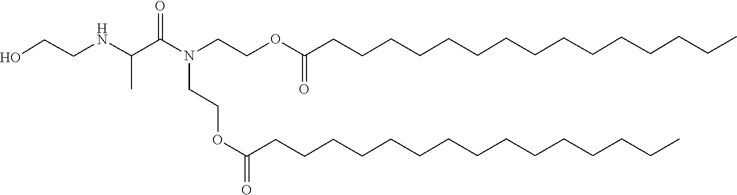 [SMILES: CCCCCCCCCCCCCCCC(=O)OCCN(CCOC(=O)CCCCCCCCCCCCCCC)C(=O)C(C)NCCO]

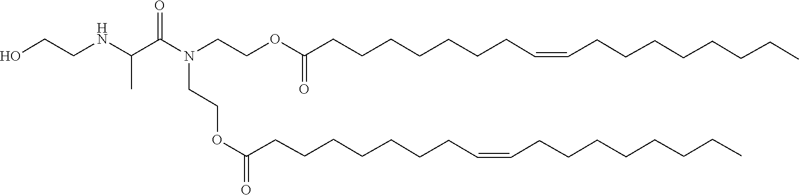 [SMILES: CCCCCCCC/C=C\CCCCCCCC(=O)OCCN(CCOC(=O)CCCCCCC/C=C\CCCCCCCC)C(=O)C(C)NCCO]

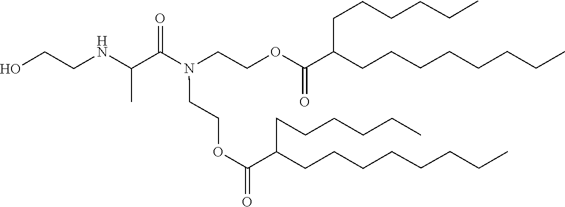 [SMILES: CCCCCCCCC(CCCCCC)C(=O)OCCN(CCOC(=O)C(CCCCCC)CCCCCCCC)C(=O)C(C)NCCO]

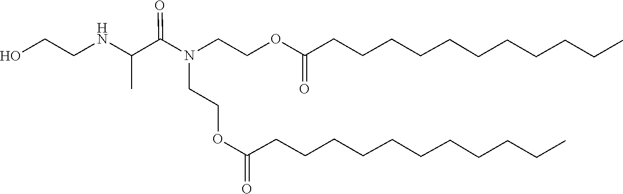 [SMILES: CCCCCCCCCCCC(=O)OCCN(CCOC(=O)CCCCCCCCCCC)C(=O)C(C)NCCO]

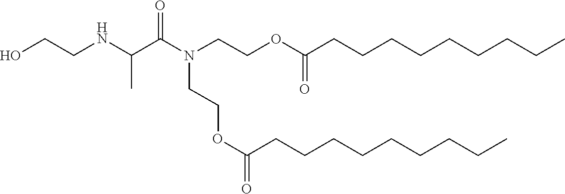 [SMILES: CCCCCCCCCC(=O)OCCN(CCOC(=O)CCCCCCCCC)C(=O)C(C)NCCO]

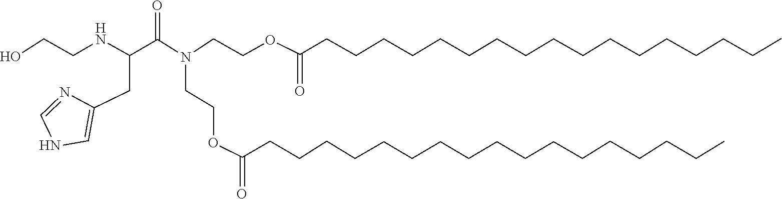 [SMILES: CCCCCCCCCCCCCCCCCC(=O)OCCN(CCOC(=O)CCCCCCCCCCCCCCCCC)C(=O)C(Cc1c[nH]cn1)NCCO]

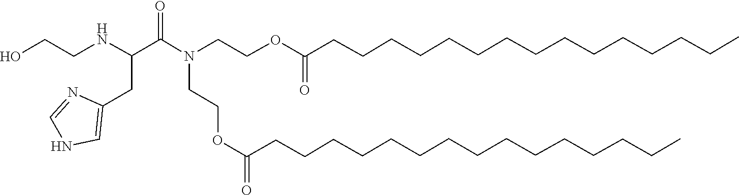 [SMILES: CCCCCCCCCCCCCCCC(=O)OCCN(CCOC(=O)CCCCCCCCCCCCCCC)C(=O)C(Cc1c[nH]cn1)NCCO]

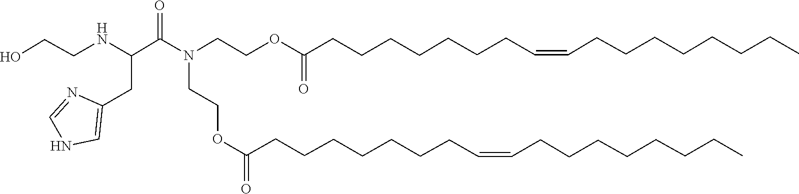 [SMILES: CCCCCCCC/C=C\CCCCCCCC(=O)OCCN(CCOC(=O)CCCCCCC/C=C\CCCCCCCC)C(=O)C(Cc1c[nH]cn1)NCCO]

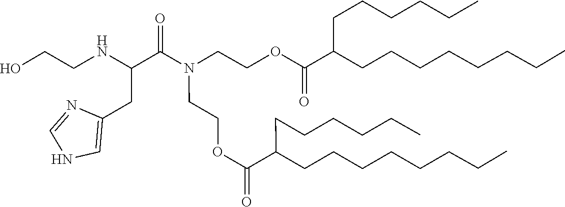 [SMILES: CCCCCCCCC(CCCCCC)C(=O)OCCN(CCOC(=O)C(CCCCCC)CCCCCCCC)C(=O)C(Cc1c[nH]cn1)NCCO]

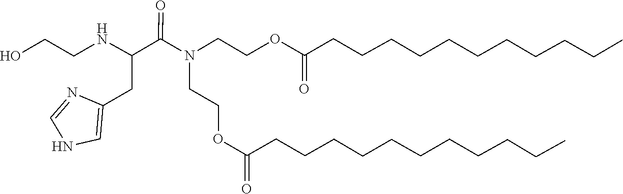 [SMILES: CCCCCCCCCCCC(=O)OCCN(CCOC(=O)CCCCCCCCCCC)C(=O)C(Cc1c[nH]cn1)NCCO]

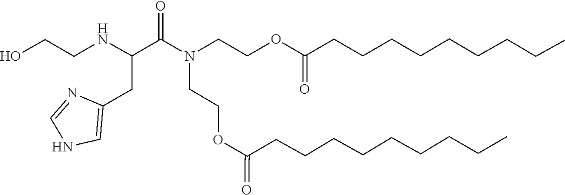 [SMILES: CCCCCCCCCC(=O)OCCN(CCOC(=O)CCCCCCCCC)C(=O)C(Cc1c[nH]cn1)NCCO]

Representative Series 600 lipidoids include those in the table below:

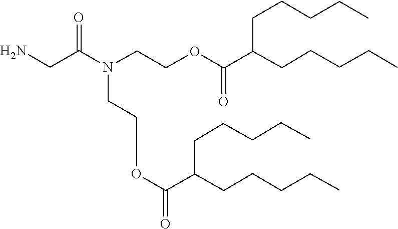 [SMILES: CCCCCC(CCCCC)C(=O)OCCN(CCOC(=O)C(CCCCC)CCCCC)C(=O)CN]

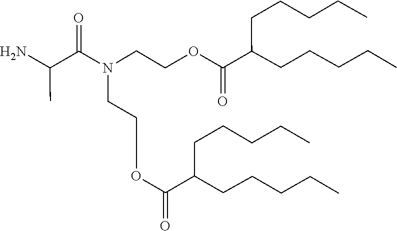 [SMILES: CCCCCC(CCCCC)C(=O)OCCN(CCOC(=O)C(CCCCC)CCCCC)C(=O)C(C)N]

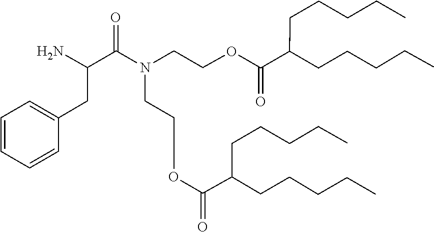 [SMILES: CCCCCC(CCCCC)C(=O)OCCN(CCOC(=O)C(CCCCC)CCCCC)C(=O)C(N)Cc1ccccc1]

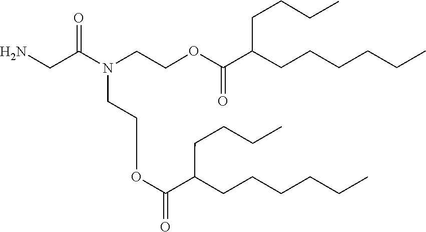 [SMILES: CCCCCCC(CCCC)C(=O)OCCN(CCOC(=O)C(CCCC)CCCCCC)C(=O)CN]

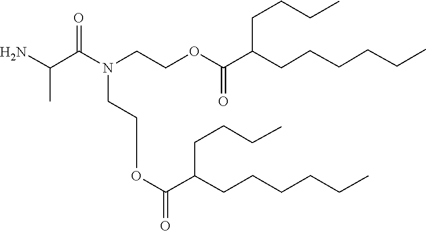 [SMILES: CCCCCCC(CCCC)C(=O)OCCN(CCOC(=O)C(CCCC)CCCCCC)C(=O)C(C)N]

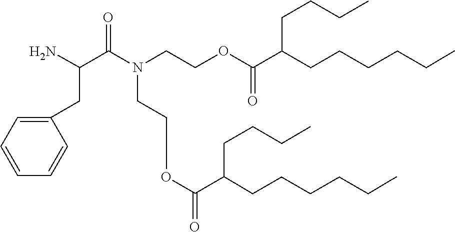 [SMILES: CCCCCCC(CCCC)C(=O)OCCN(CCOC(=O)C(CCCC)CCCCCC)C(=O)C(N)Cc1ccccc1]

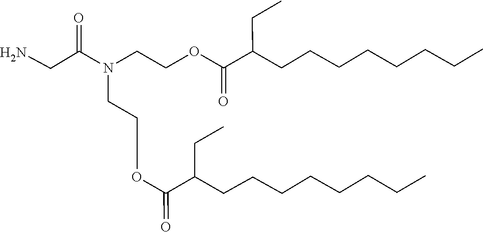 [SMILES: CCCCCCCCC(CC)C(=O)OCCN(CCOC(=O)C(CC)CCCCCCCC)C(=O)CN]

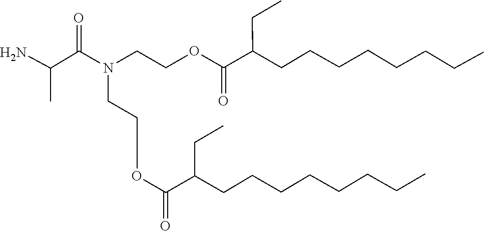 [SMILES: CCCCCCCCC(CC)C(=O)OCCN(CCOC(=O)C(CC)CCCCCCCC)C(=O)C(C)N]

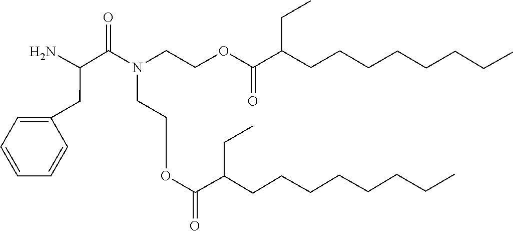 [SMILES: CCCCCCCCC(CC)C(=O)OCCN(CCOC(=O)C(CC)CCCCCCCC)C(=O)C(N)Cc1ccccc1]

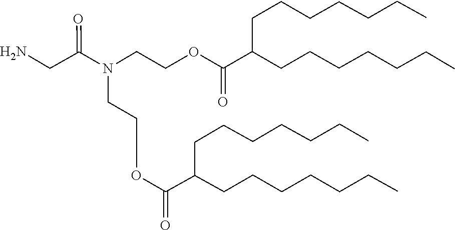 [SMILES: CCCCCCCC(CCCCCCC)C(=O)OCCN(CCOC(=O)C(CCCCCCC)CCCCCCC)C(=O)CN]

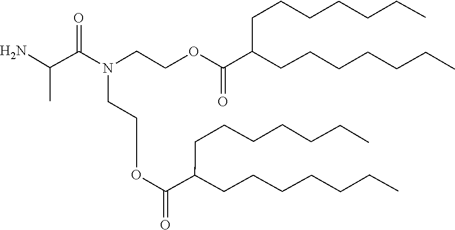 [SMILES: CCCCCCCC(CCCCCCC)C(=O)OCCN(CCOC(=O)C(CCCCCCC)CCCCCCC)C(=O)C(C)N]

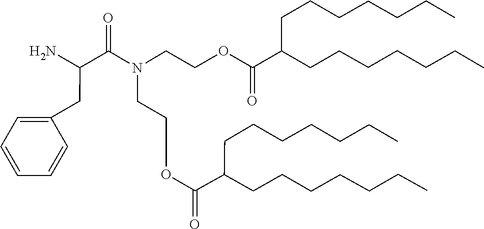 [SMILES: CCCCCCCC(CCCCCCC)C(=O)OCCN(CCOC(=O)C(CCCCCCC)CCCCCCC)C(=O)C(N)Cc1ccccc1]

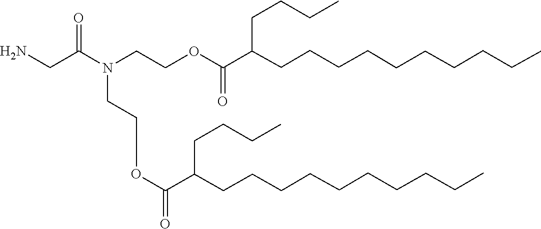 [SMILES: CCCCCCCCCCC(CCCC)C(=O)OCCN(CCOC(=O)C(CCCC)CCCCCCCCCC)C(=O)CN]

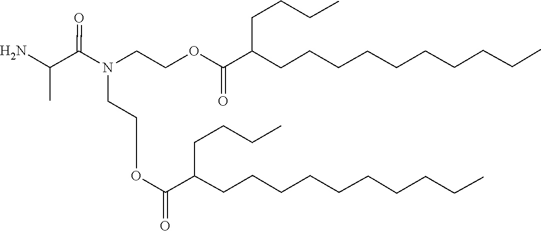 [SMILES: CCCCCCCCCCC(CCCC)C(=O)OCCN(CCOC(=O)C(CCCC)CCCCCCCCCC)C(=O)C(C)N]

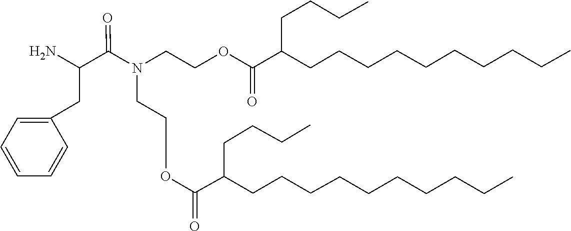 [SMILES: CCCCCCCCCCC(CCCC)C(=O)OCCN(CCOC(=O)C(CCCC)CCCCCCCCCC)C(=O)C(N)Cc1ccccc1]

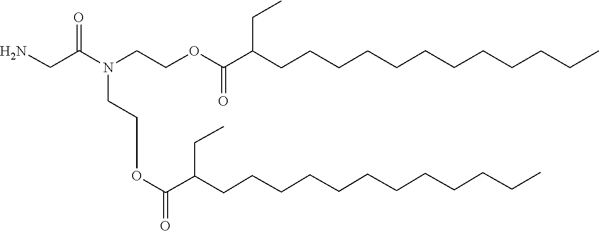 [SMILES: CCCCCCCCCCCCC(CC)C(=O)OCCN(CCOC(=O)C(CC)CCCCCCCCCCCC)C(=O)CN]

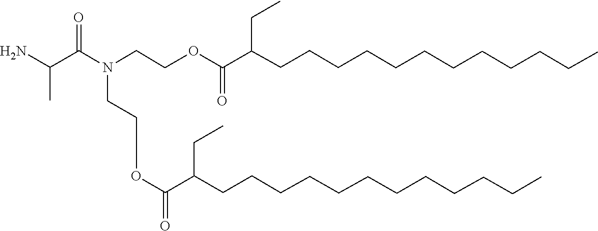 [SMILES: CCCCCCCCCCCCC(CC)C(=O)OCCN(CCOC(=O)C(CC)CCCCCCCCCCCC)C(=O)C(C)N]

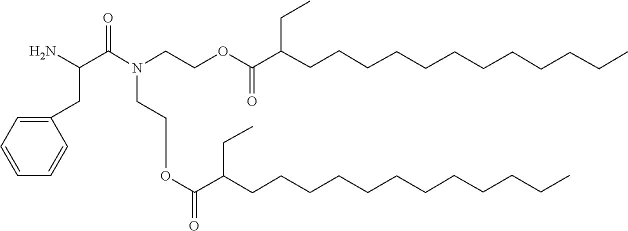 [SMILES: CCCCCCCCCCCCC(CC)C(=O)OCCN(CCOC(=O)C(CC)CCCCCCCCCCCC)C(=O)C(N)Cc1ccccc1]

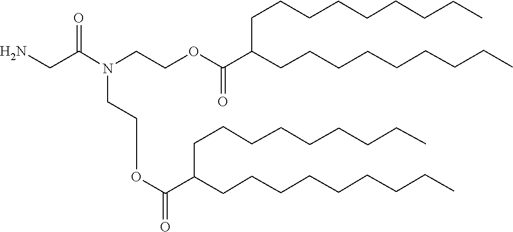 [SMILES: CCCCCCCCCC(CCCCCCCCC)C(=O)OCCN(CCOC(=O)C(CCCCCCCCC)CCCCCCCCC)C(=O)CN]

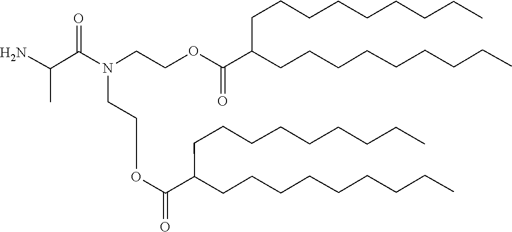 [SMILES: CCCCCCCCCC(CCCCCCCCC)C(=O)OCCN(CCOC(=O)C(CCCCCCCCC)CCCCCCCCC)C(=O)C(C)N]

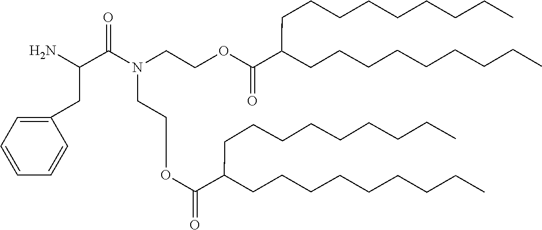 [SMILES: CCCCCCCCCC(CCCCCCCCC)C(=O)OCCN(CCOC(=O)C(CCCCCCCCC)CCCCCCCCC)C(=O)C(N)Cc1ccccc1]

Representative Series 800 lipidoids include those in the table below

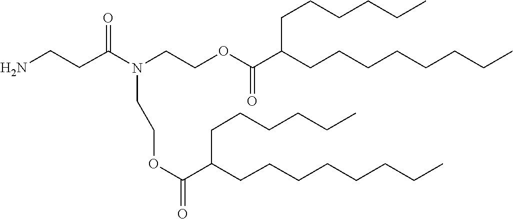 [SMILES: CCCCCCCCC(CCCCCC)C(=O)OCCN(CCOC(=O)C(CCCCCC)CCCCCCCC)C(=O)CCN]

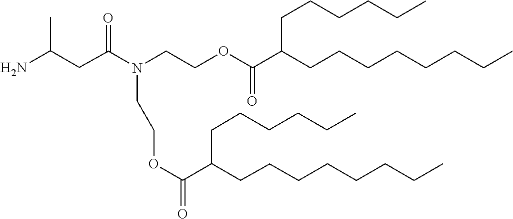 [SMILES: CCCCCCCCC(CCCCCC)C(=O)OCCN(CCOC(=O)C(CCCCCC)CCCCCCCC)C(=O)CC(C)N]

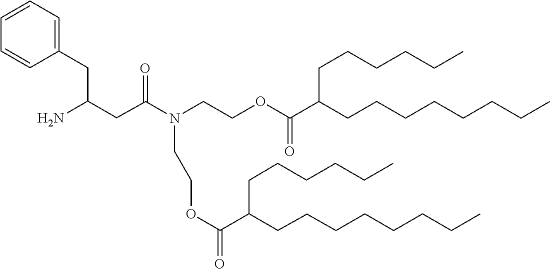 [SMILES: CCCCCCCCC(CCCCCC)C(=O)OCCN(CCOC(=O)C(CCCCCC)CCCCCCCC)C(=O)CC(N)Cc1ccccc1]

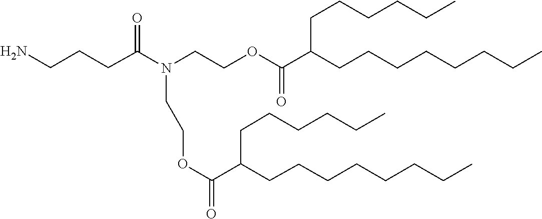 [SMILES: CCCCCCCCC(CCCCCC)C(=O)OCCN(CCOC(=O)C(CCCCCC)CCCCCCCC)C(=O)CCCN]

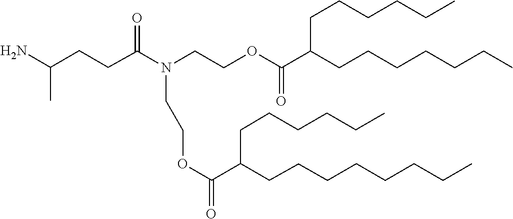 [SMILES: CCCCCCCCC(CCCCCC)C(=O)OCCN(CCOC(=O)C(CCCCCC)CCCCCCC)C(=O)CCC(C)N]

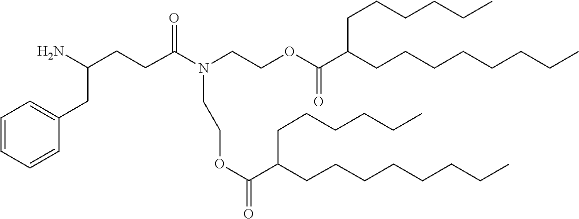 [SMILES: CCCCCCCCC(CCCCCC)C(=O)OCCN(CCOC(=O)C(CCCCCC)CCCCCCCC)C(=O)CCC(N)Cc1ccccc1]

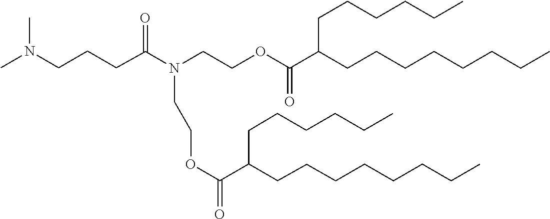 [SMILES: CCCCCCCCC(CCCCCC)C(=O)OCCN(CCOC(=O)C(CCCCCC)CCCCCCCC)C(=O)CCCN(C)C]

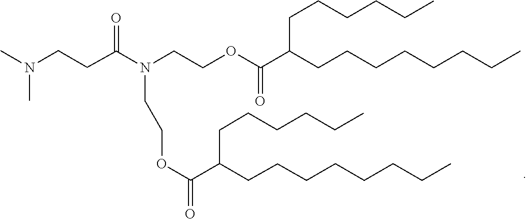 [SMILES: CCCCCCCCC(CCCCCC)C(=O)OCCN(CCOC(=O)C(CCCCCC)CCCCCCCC)C(=O)CCN(C)C]

Representative Series 900 lipidoids include those in the table below:

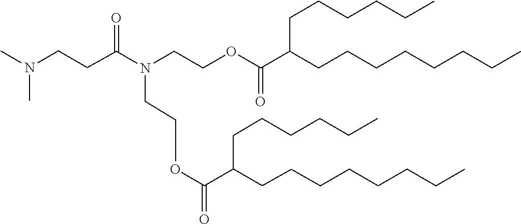 [SMILES: CCCCCCCCC(CCCCCC)C(=O)OCCN(CCOC(=O)C(CCCCCC)CCCCCCCC)C(=O)CCN(C)C]

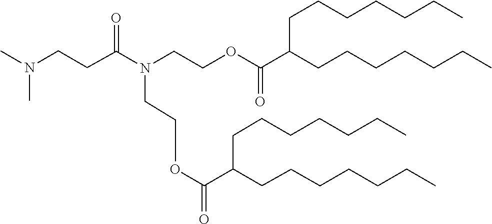 [SMILES: CCCCCCCC(CCCCCCC)C(=O)OCCN(CCOC(=O)C(CCCCCCC)CCCCCCC)C(=O)CCN(C)C]

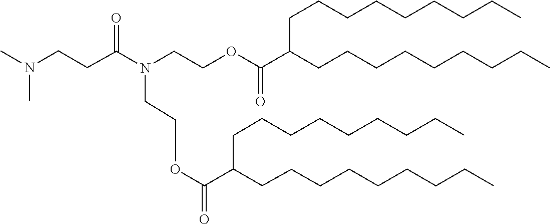 [SMILES: CCCCCCCCCC(CCCCCCCCC)C(=O)OCCN(CCOC(=O)C(CCCCCCCCC)CCCCCCCCC)C(=O)CCN(C)C]

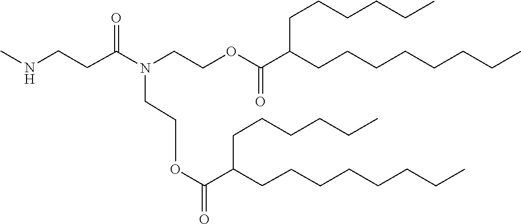 [SMILES: CCCCCCCCC(CCCCCC)C(=O)OCCN(CCOC(=O)C(CCCCCC)CCCCCCCC)C(=O)CCNC]

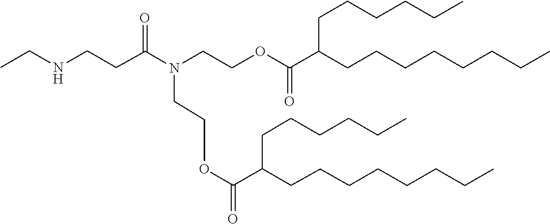 [SMILES: CCCCCCCCC(CCCCCC)C(=O)OCCN(CCOC(=O)C(CCCCCC)CCCCCCCC)C(=O)CCNCC]

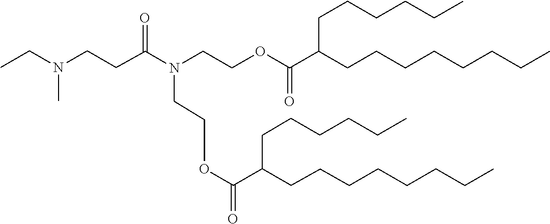 [SMILES: CCCCCCCCC(CCCCCC)C(=O)OCCN(CCOC(=O)C(CCCCCC)CCCCCCCC)C(=O)CCN(C)CC]

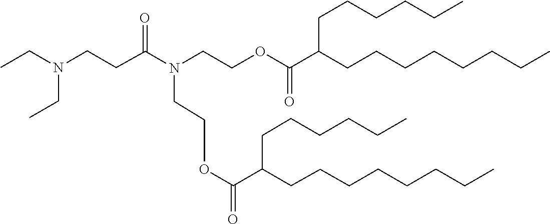 [SMILES: CCCCCCCCC(CCCCCC)C(=O)OCCN(CCOC(=O)C(CCCCCC)CCCCCCCC)C(=O)CCN(CC)CC]

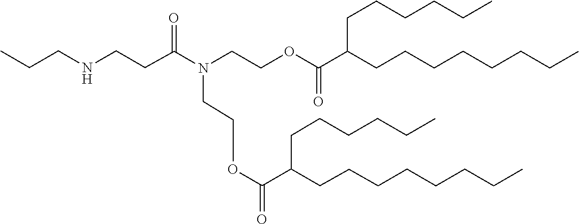 [SMILES: CCCCCCCCC(CCCCCC)C(=O)OCCN(CCOC(=O)C(CCCCCC)CCCCCCCC)C(=O)CCNCCC]

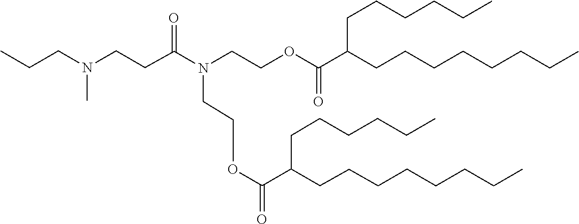 [SMILES: CCCCCCCCC(CCCCCC)C(=O)OCCN(CCOC(=O)C(CCCCCC)CCCCCCCC)C(=O)CCN(C)CCC]

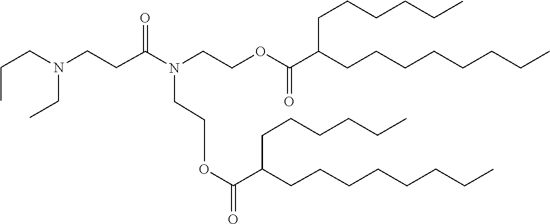 [SMILES: CCCCCCCCC(CCCCCC)C(=O)OCCN(CCOC(=O)C(CCCCCC)CCCCCCCC)C(=O)CCN(CC)CCC]

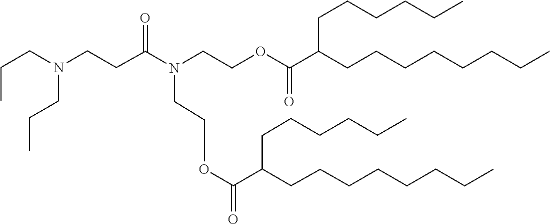 [SMILES: CCCCCCCCC(CCCCCC)C(=O)OCCN(CCOC(=O)C(CCCCCC)CCCCCCCC)C(=O)CCN(CCC)CCC]

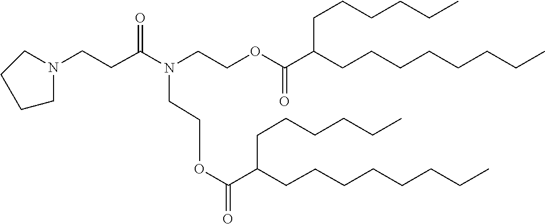 [SMILES: CCCCCCCCC(CCCCCC)C(=O)OCCN(CCOC(=O)C(CCCCCC)CCCCCCCC)C(=O)CCN1CCCC1]

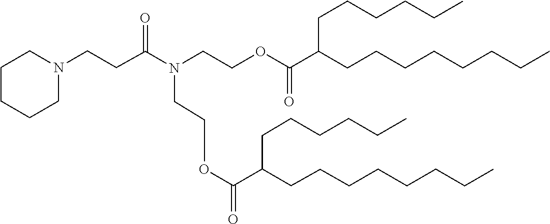 [SMILES: CCCCCCCCC(CCCCCC)C(=O)OCCN(CCOC(=O)C(CCCCCC)CCCCCCCC)C(=O)CCN1CCCCC1]

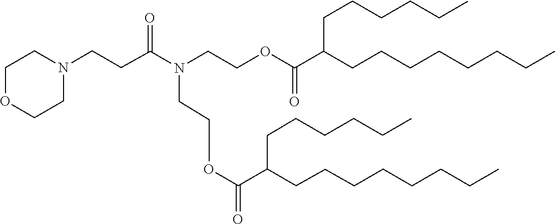 [SMILES: CCCCCCCCC(CCCCCC)C(=O)OCCN(CCOC(=O)C(CCCCCC)CCCCCCCC)C(=O)CCN1CCOCC1]

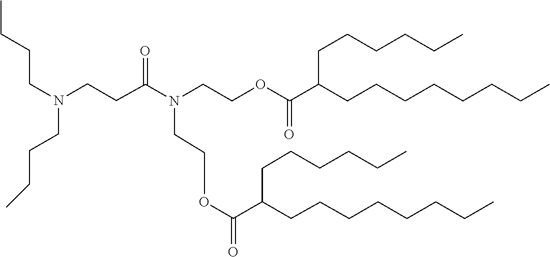 [SMILES: CCCCCCCCC(CCCCCC)C(=O)OCCN(CCOC(=O)C(CCCCCC)CCCCCCCC)C(=O)CCN(CCCC)CCCC]

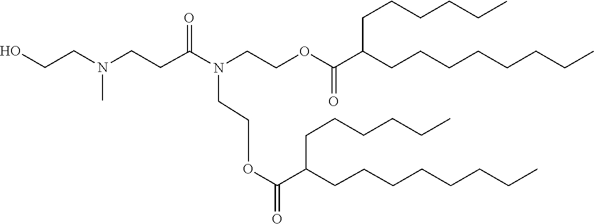 [SMILES: CCCCCCCCC(CCCCCC)C(=O)OCCN(CCOC(=O)C(CCCCCC)CCCCCCCC)C(=O)CCN(C)CCO]

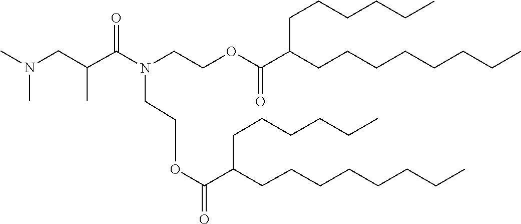 [SMILES: CCCCCCCCC(CCCCCC)C(=O)OCCN(CCOC(=O)C(CCCCCC)CCCCCCCC)C(=O)C(C)CN(C)C]

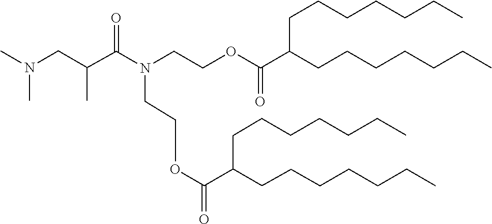 [SMILES: CCCCCCCC(CCCCCCC)C(=O)OCCN(CCOC(=O)C(CCCCCCC)CCCCCCC)C(=O)C(C)CN(C)C]

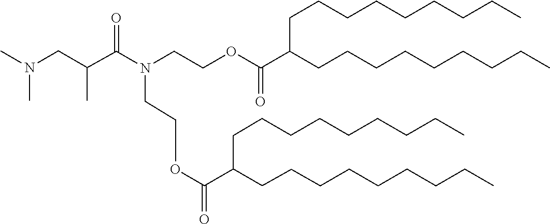 [SMILES: CCCCCCCCCC(CCCCCCCCC)C(=O)OCCN(CCOC(=O)C(CCCCCCCCC)CCCCCCCCC)C(=O)C(C)CN(C)C]

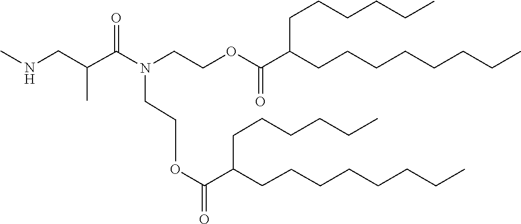 [SMILES: CCCCCCCCC(CCCCCC)C(=O)OCCN(CCOC(=O)C(CCCCCC)CCCCCCCC)C(=O)C(C)CNC]

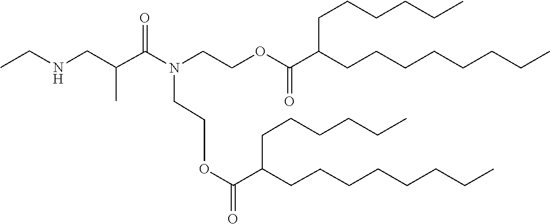 [SMILES: CCCCCCCCC(CCCCCC)C(=O)OCCN(CCOC(=O)C(CCCCCC)CCCCCCCC)C(=O)C(C)CNCC]

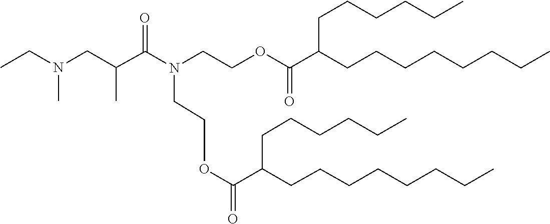 [SMILES: CCCCCCCCC(CCCCCC)C(=O)OCCN(CCOC(=O)C(CCCCCC)CCCCCCCC)C(=O)C(C)CN(C)CC]

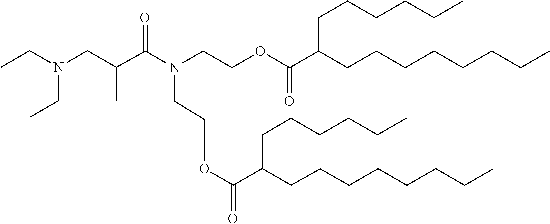 [SMILES: CCCCCCCCC(CCCCCC)C(=O)OCCN(CCOC(=O)C(CCCCCC)CCCCCCCC)C(=O)C(C)CN(CC)CC]

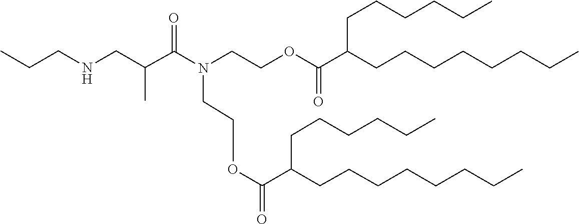 [SMILES: CCCCCCCCC(CCCCCC)C(=O)OCCN(CCOC(=O)C(CCCCCC)CCCCCCCC)C(=O)C(C)CNCCC]

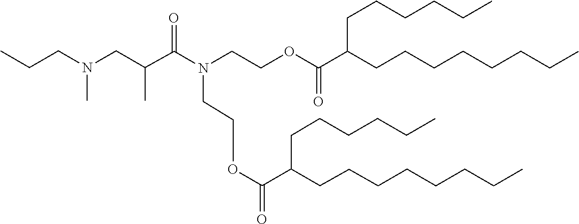 [SMILES: CCCCCCCCC(CCCCCC)C(=O)OCCN(CCOC(=O)C(CCCCCC)CCCCCCCC)C(=O)C(C)CN(C)CCC]

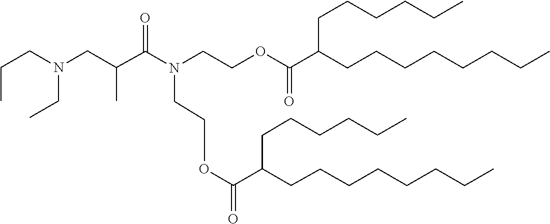 [SMILES: CCCCCCCCC(CCCCCC)C(=O)OCCN(CCOC(=O)C(CCCCCC)CCCCCCCC)C(=O)C(C)CN(CC)CCC]

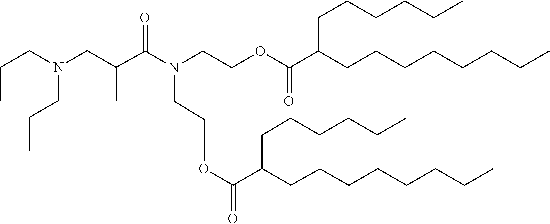 [SMILES: CCCCCCCCC(CCCCCC)C(=O)OCCN(CCOC(=O)C(CCCCCC)CCCCCCCC)C(=O)C(C)CN(CCC)CCC]

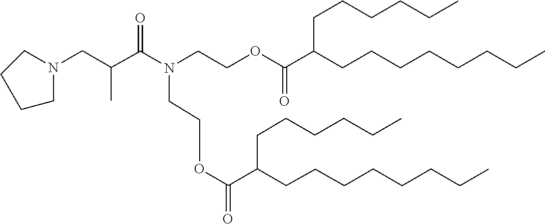 [SMILES: CCCCCCCCC(CCCCCC)C(=O)OCCN(CCOC(=O)C(CCCCCC)CCCCCCCC)C(=O)C(C)CN1CCCC1]

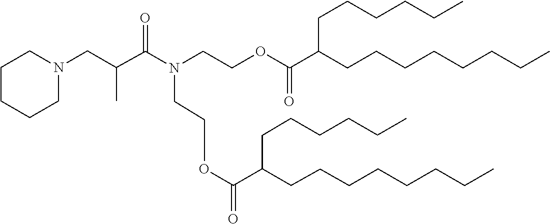 [SMILES: CCCCCCCCC(CCCCCC)C(=O)OCCN(CCOC(=O)C(CCCCCC)CCCCCCCC)C(=O)C(C)CN1CCCCC1]

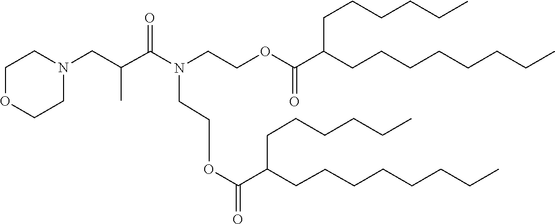 [SMILES: CCCCCCCCC(CCCCCC)C(=O)OCCN(CCOC(=O)C(CCCCCC)CCCCCCCC)C(=O)C(C)CN1CCOCC1]

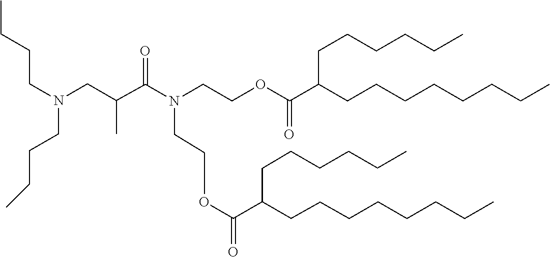 [SMILES: CCCCCCCCC(CCCCCC)C(=O)OCCN(CCOC(=O)C(CCCCCC)CCCCCCCC)C(=O)C(C)CN(CCCC)CCCC]

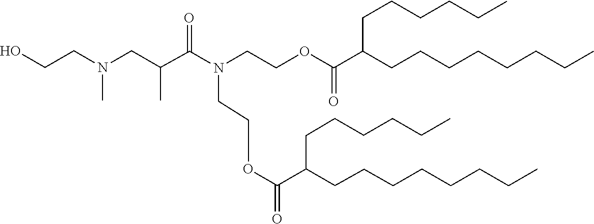 [SMILES: CCCCCCCCC(CCCCCC)C(=O)OCCN(CCOC(=O)C(CCCCCC)CCCCCCCC)C(=O)C(C)CN(C)CCO]

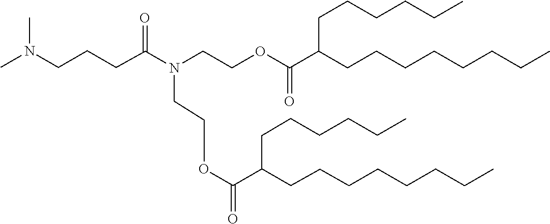 [SMILES: CCCCCCCCC(CCCCCC)C(=O)OCCN(CCOC(=O)C(CCCCCC)CCCCCCCC)C(=O)CCCN(C)C]

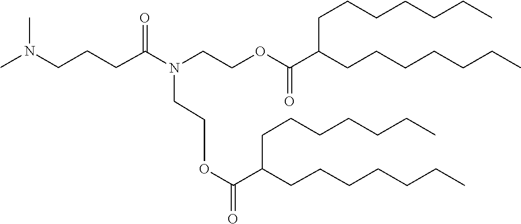 [SMILES: CCCCCCCC(CCCCCCC)C(=O)OCCN(CCOC(=O)C(CCCCCCC)CCCCCCC)C(=O)CCCN(C)C]

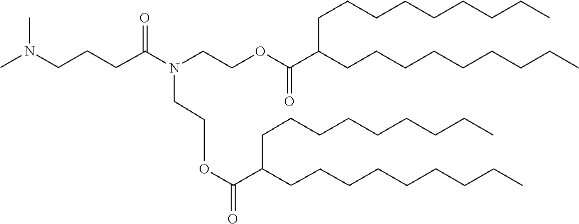 [SMILES: CCCCCCCCCC(CCCCCCCCC)C(=O)OCCN(CCOC(=O)C(CCCCCCCCC)CCCCCCCCC)C(=O)CCCN(C)C]

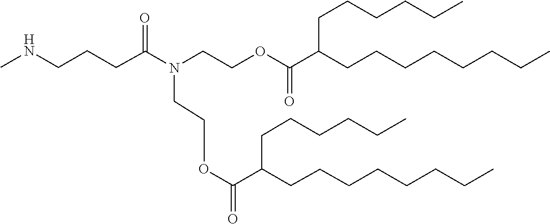 [SMILES: CCCCCCCCC(CCCCCC)C(=O)OCCN(CCOC(=O)C(CCCCCC)CCCCCCCC)C(=O)CCCNC]

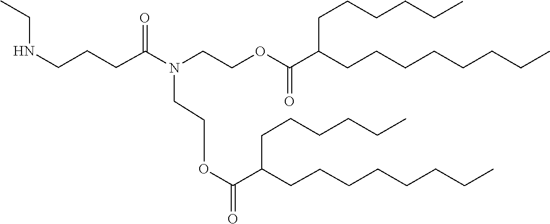 [SMILES: CCCCCCCCC(CCCCCC)C(=O)OCCN(CCOC(=O)C(CCCCCC)CCCCCCCC)C(=O)CCCNCC]

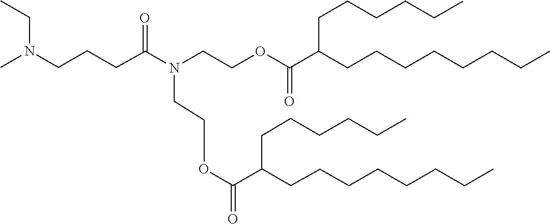 [SMILES: CCCCCCCCC(CCCCCC)C(=O)OCCN(CCOC(=O)C(CCCCCC)CCCCCCCC)C(=O)CCCN(C)CC]

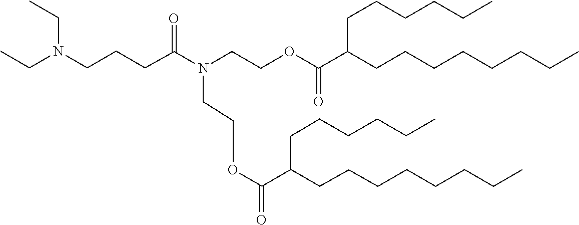 [SMILES: CCCCCCCCC(CCCCCC)C(=O)OCCN(CCOC(=O)C(CCCCCC)CCCCCCCC)C(=O)CCCN(CC)CC]

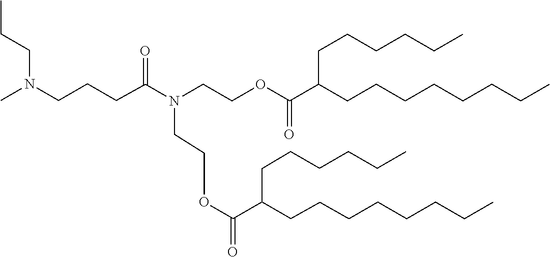 [SMILES: CCCCCCCCC(CCCCCC)C(=O)OCCN(CCOC(=O)C(CCCCCC)CCCCCCCC)C(=O)CCCN(C)CCC]

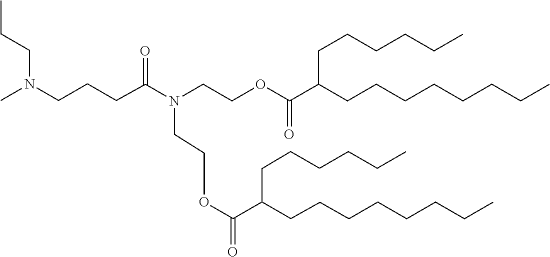 [SMILES: CCCCCCCCC(CCCCCC)C(=O)OCCN(CCOC(=O)C(CCCCCC)CCCCCCCC)C(=O)CCCN(C)CCC]

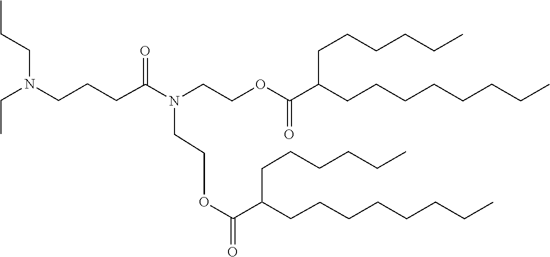 [SMILES: CCCCCCCCC(CCCCCC)C(=O)OCCN(CCOC(=O)C(CCCCCC)CCCCCCCC)C(=O)CCCN(CC)CCC]

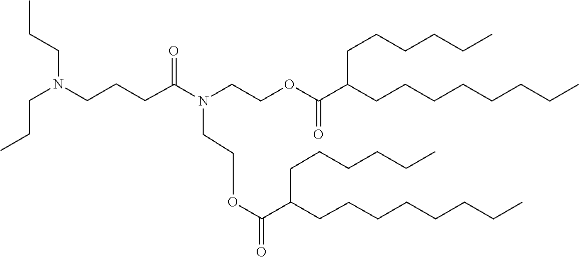 [SMILES: CCCCCCCCC(CCCCCC)C(=O)OCCN(CCOC(=O)C(CCCCCC)CCCCCCCC)C(=O)CCCN(CCC)CCC]

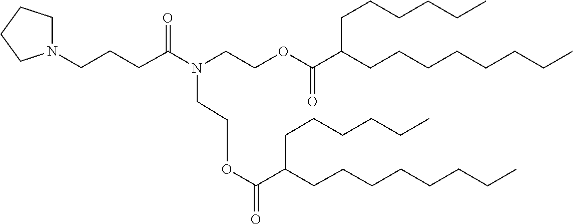 [SMILES: CCCCCCCCC(CCCCCC)C(=O)OCCN(CCOC(=O)C(CCCCCC)CCCCCCCC)C(=O)CCCN1CCCC1]

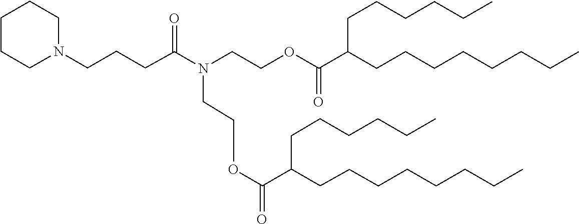 [SMILES: CCCCCCCCC(CCCCCC)C(=O)OCCN(CCOC(=O)C(CCCCCC)CCCCCCCC)C(=O)CCCN1CCCCC1]

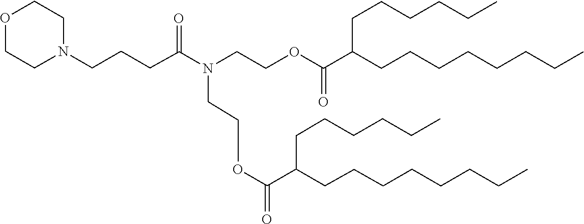 [SMILES: CCCCCCCCC(CCCCCC)C(=O)OCCN(CCOC(=O)C(CCCCCC)CCCCCCCC)C(=O)CCCN1CCOCC1]

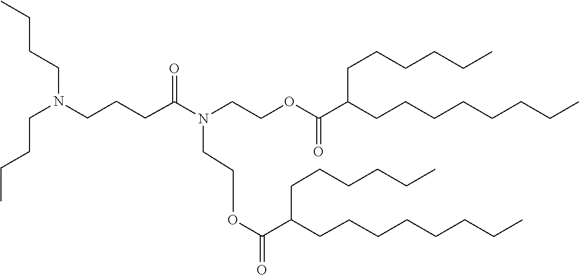 [SMILES: CCCCCCCCC(CCCCCC)C(=O)OCCN(CCOC(=O)C(CCCCCC)CCCCCCCC)C(=O)CCCN(CCCC)CCCC]

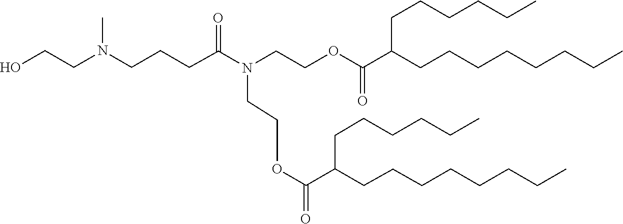 [SMILES: CCCCCCCCC(CCCCCC)C(=O)OCCN(CCOC(=O)C(CCCCCC)CCCCCCCC)C(=O)CCCN(C)CCO]

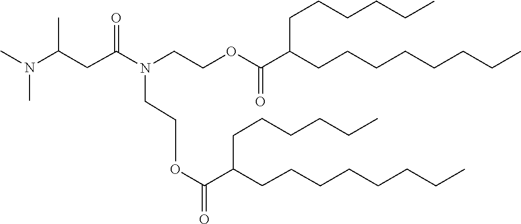 [SMILES: CCCCCCCCC(CCCCCC)C(=O)OCCN(CCOC(=O)C(CCCCCC)CCCCCCCC)C(=O)CC(C)N(C)C]

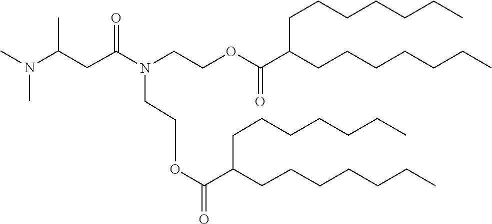 [SMILES: CCCCCCCC(CCCCCCC)C(=O)OCCN(CCOC(=O)C(CCCCCCC)CCCCCCC)C(=O)CC(C)N(C)C]

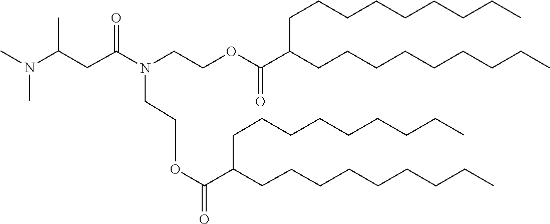 [SMILES: CCCCCCCCCC(CCCCCCCCC)C(=O)OCCN(CCOC(=O)C(CCCCCCCCC)CCCCCCCCC)C(=O)CC(C)N(C)C]

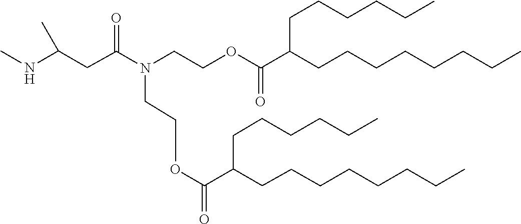 [SMILES: CCCCCCCCC(CCCCCC)C(=O)OCCN(CCOC(=O)C(CCCCCC)CCCCCCCC)C(=O)CC(C)NC]

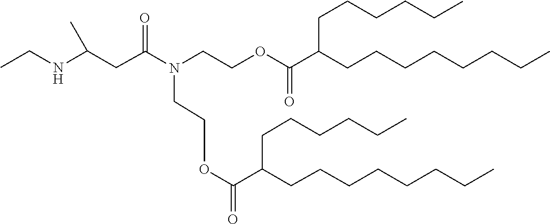 [SMILES: CCCCCCCCC(CCCCCC)C(=O)OCCN(CCOC(=O)C(CCCCCC)CCCCCCCC)C(=O)CC(C)NCC]

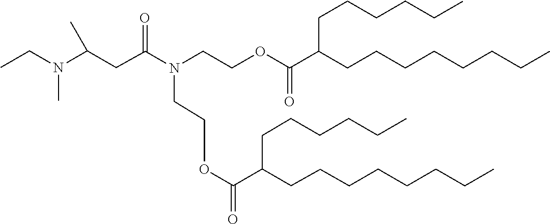 [SMILES: CCCCCCCCC(CCCCCC)C(=O)OCCN(CCOC(=O)C(CCCCCC)CCCCCCCC)C(=O)CC(C)N(C)CC]

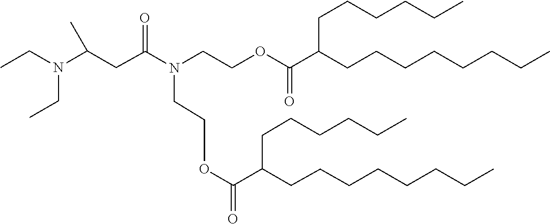 [SMILES: CCCCCCCCC(CCCCCC)C(=O)OCCN(CCOC(=O)C(CCCCCC)CCCCCCCC)C(=O)CC(C)N(CC)CC]

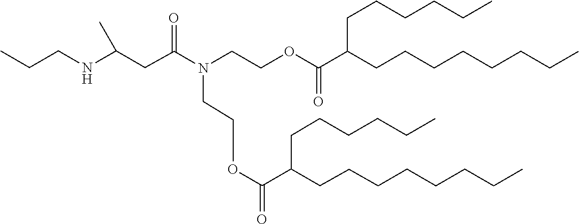 [SMILES: CCCCCCCCC(CCCCCC)C(=O)OCCN(CCOC(=O)C(CCCCCC)CCCCCCCC)C(=O)CC(C)NCCC]

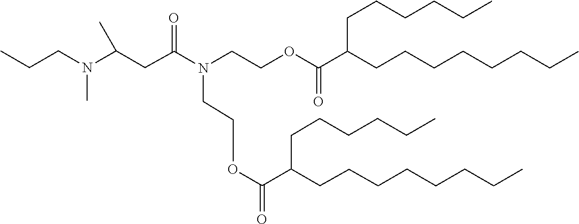 [SMILES: CCCCCCCCC(CCCCCC)C(=O)OCCN(CCOC(=O)C(CCCCCC)CCCCCCCC)C(=O)CC(C)N(C)CCC]

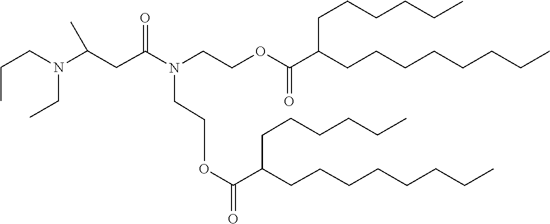 [SMILES: CCCCCCCCC(CCCCCC)C(=O)OCCN(CCOC(=O)C(CCCCCC)CCCCCCCC)C(=O)CC(C)N(CC)CCC]

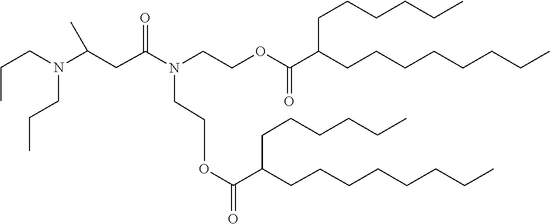 [SMILES: CCCCCCCCC(CCCCCC)C(=O)OCCN(CCOC(=O)C(CCCCCC)CCCCCCCC)C(=O)CC(C)N(CCC)CCC]

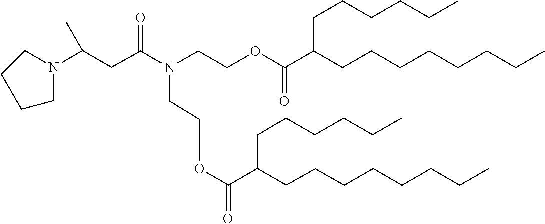 [SMILES: CCCCCCCCC(CCCCCC)C(=O)OCCN(CCOC(=O)C(CCCCCC)CCCCCCCC)C(=O)CC(C)N1CCCC1]

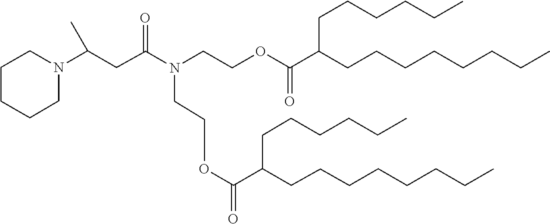 [SMILES: CCCCCCCCC(CCCCCC)C(=O)OCCN(CCOC(=O)C(CCCCCC)CCCCCCCC)C(=O)CC(C)N1CCCCC1]

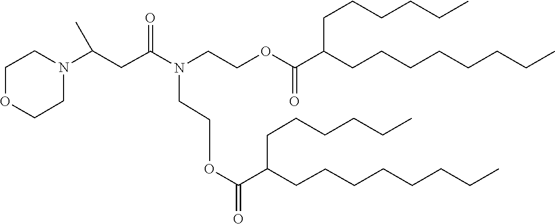 [SMILES: CCCCCCCCC(CCCCCC)C(=O)OCCN(CCOC(=O)C(CCCCCC)CCCCCCCC)C(=O)CC(C)N1CCOCC1]

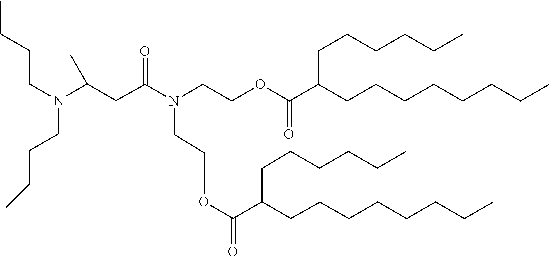 [SMILES: CCCCCCCCC(CCCCCC)C(=O)OCCN(CCOC(=O)C(CCCCCC)CCCCCCCC)C(=O)CC(C)N(CCCC)CCCC]

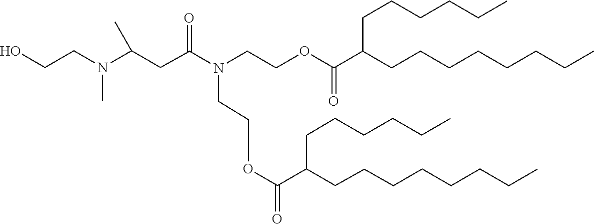 [SMILES: CCCCCCCCC(CCCCCC)C(=O)OCCN(CCOC(=O)C(CCCCCC)CCCCCCCC)C(=O)CC(C)N(C)CCO]

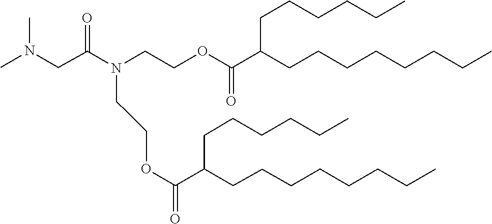 [SMILES: CCCCCCCCC(CCCCCC)C(=O)OCCN(CCOC(=O)C(CCCCCC)CCCCCCCC)C(=O)CN(C)C]

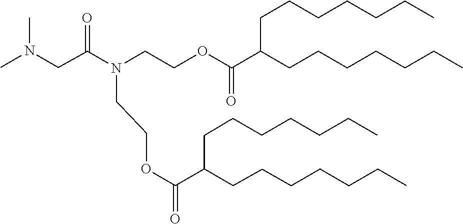 [SMILES: CCCCCCCC(CCCCCCC)C(=O)OCCN(CCOC(=O)C(CCCCCCC)CCCCCCC)C(=O)CN(C)C]

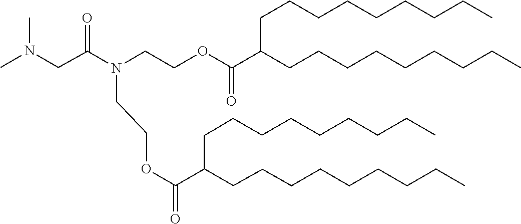 [SMILES: CCCCCCCCCC(CCCCCCCCC)C(=O)OCCN(CCOC(=O)C(CCCCCCCCC)CCCCCCCCC)C(=O)CN(C)C]

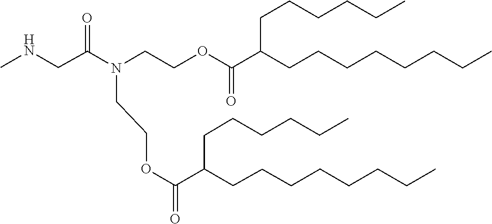 [SMILES: CCCCCCCCC(CCCCCC)C(=O)OCCN(CCOC(=O)C(CCCCCC)CCCCCCCC)C(=O)CNC]

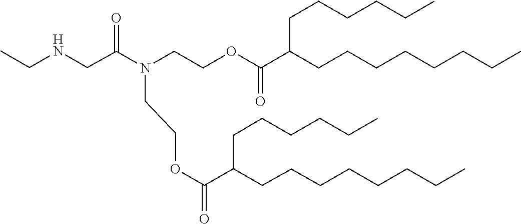 [SMILES: CCCCCCCCC(CCCCCC)C(=O)OCCN(CCOC(=O)C(CCCCCC)CCCCCCCC)C(=O)CNCC]

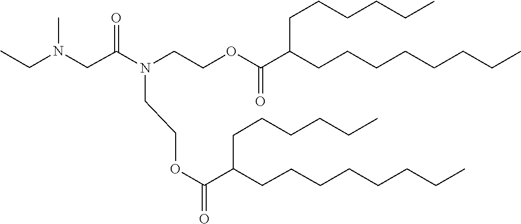 [SMILES: CCCCCCCCC(CCCCCC)C(=O)OCCN(CCOC(=O)C(CCCCCC)CCCCCCCC)C(=O)CN(C)CC]

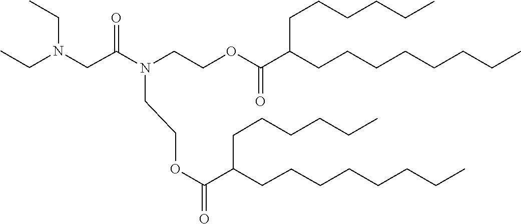 [SMILES: CCCCCCCCC(CCCCCC)C(=O)OCCN(CCOC(=O)C(CCCCCC)CCCCCCCC)C(=O)CN(CC)CC]

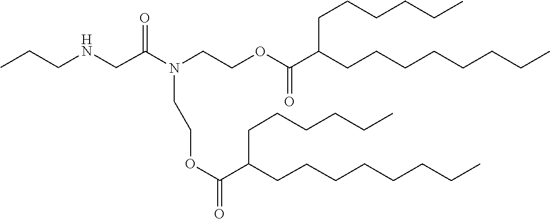 [SMILES: CCCCCCCCC(CCCCCC)C(=O)OCCN(CCOC(=O)C(CCCCCC)CCCCCCCC)C(=O)CNCCC]

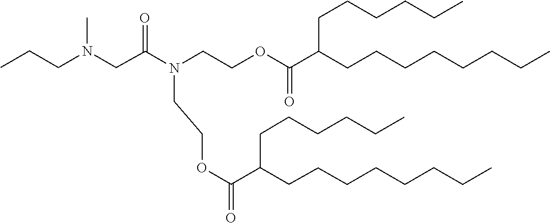 [SMILES: CCCCCCCCC(CCCCCC)C(=O)OCCN(CCOC(=O)C(CCCCCC)CCCCCCCC)C(=O)CN(C)CCC]

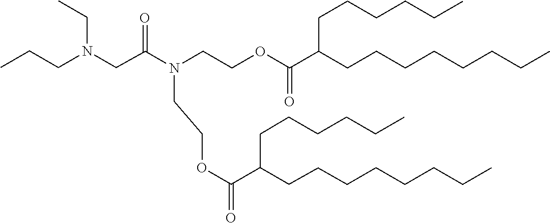 [SMILES: CCCCCCCCC(CCCCCC)C(=O)OCCN(CCOC(=O)C(CCCCCC)CCCCCCCC)C(=O)CN(CC)CCC]

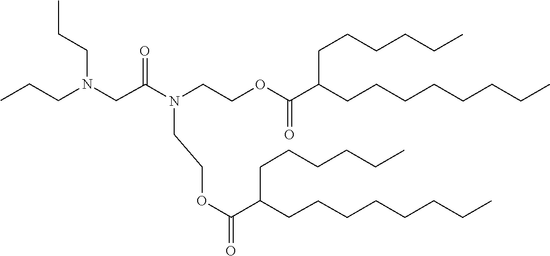 [SMILES: CCCCCCCCC(CCCCCC)C(=O)OCCN(CCOC(=O)C(CCCCCC)CCCCCCCC)C(=O)CN(CCC)CCC]

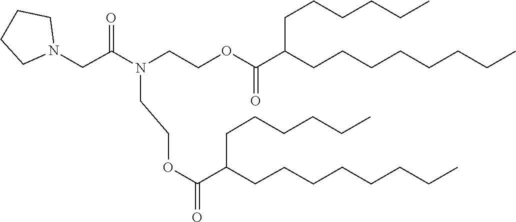 [SMILES: CCCCCCCCC(CCCCCC)C(=O)OCCN(CCOC(=O)C(CCCCCC)CCCCCCCC)C(=O)CN1CCCC1]

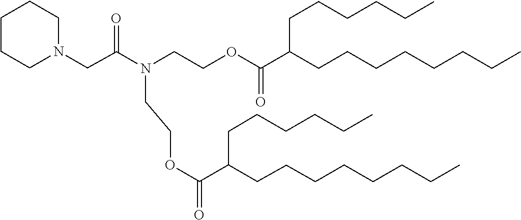 [SMILES: CCCCCCCCC(CCCCCC)C(=O)OCCN(CCOC(=O)C(CCCCCC)CCCCCCCC)C(=O)CN1CCCCC1]

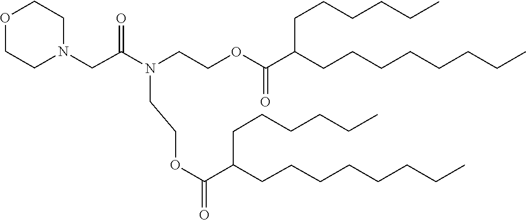 [SMILES: CCCCCCCCC(CCCCCC)C(=O)OCCN(CCOC(=O)C(CCCCCC)CCCCCCCC)C(=O)CN1CCOCC1]

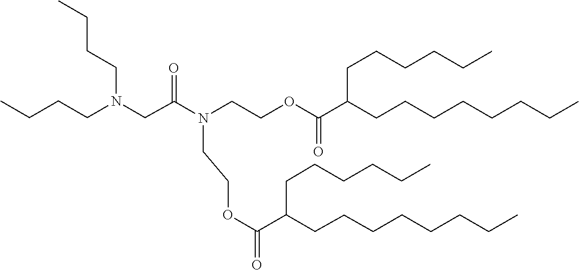 [SMILES: CCCCCCCCC(CCCCCC)C(=O)OCCN(CCOC(=O)C(CCCCCC)CCCCCCCC)C(=O)CN(CCCC)CCCC]

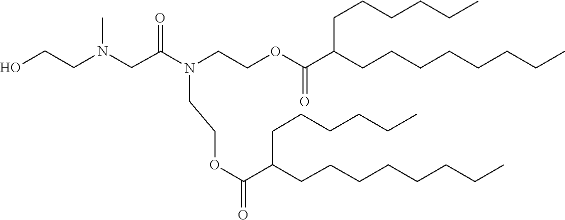 [SMILES: CCCCCCCCC(CCCCCC)C(=O)OCCN(CCOC(=O)C(CCCCCC)CCCCCCCC)C(=O)CN(C)CCO]

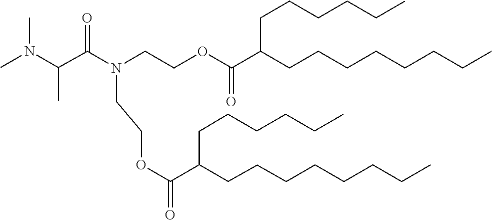 [SMILES: CCCCCCCCC(CCCCCC)C(=O)OCCN(CCOC(=O)C(CCCCCC)CCCCCCCC)C(=O)C(C)N(C)C]

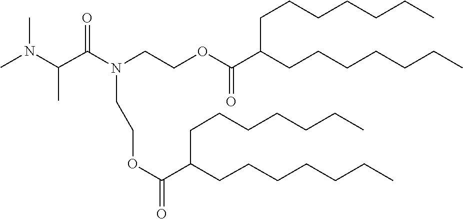 [SMILES: CCCCCCCC(CCCCCCC)C(=O)OCCN(CCOC(=O)C(CCCCCCC)CCCCCCC)C(=O)C(C)N(C)C]

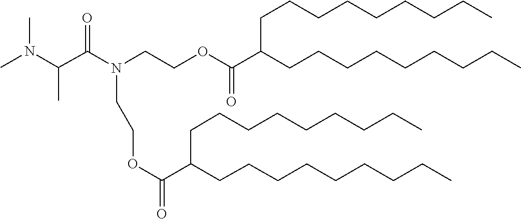 [SMILES: CCCCCCCCCC(CCCCCCCCC)C(=O)OCCN(CCOC(=O)C(CCCCCCCCC)CCCCCCCCC)C(=O)C(C)N(C)C]

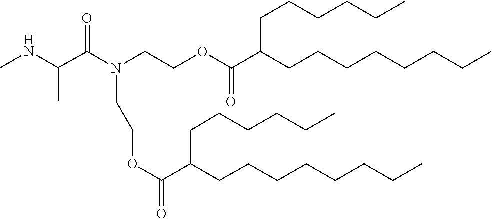 [SMILES: CCCCCCCCC(CCCCCC)C(=O)OCCN(CCOC(=O)C(CCCCCC)CCCCCCCC)C(=O)C(C)NC]

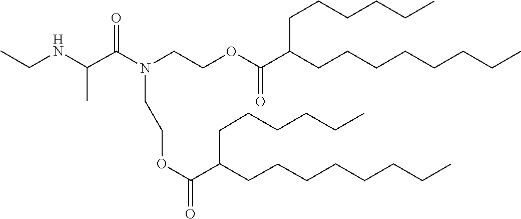 [SMILES: CCCCCCCCC(CCCCCC)C(=O)OCCN(CCOC(=O)C(CCCCCC)CCCCCCCC)C(=O)C(C)NCC]

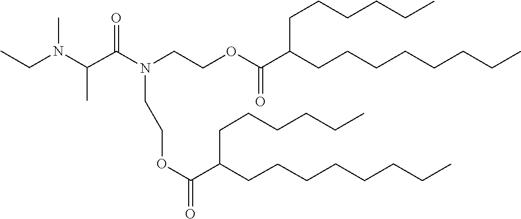 [SMILES: CCCCCCCCC(CCCCCC)C(=O)OCCN(CCOC(=O)C(CCCCCC)CCCCCCCC)C(=O)C(C)N(C)CC]

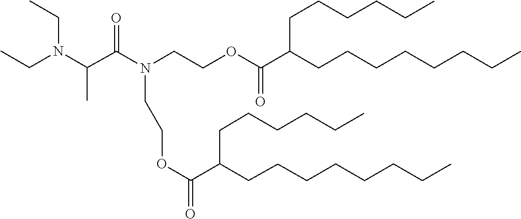 [SMILES: CCCCCCCCC(CCCCCC)C(=O)OCCN(CCOC(=O)C(CCCCCC)CCCCCCCC)C(=O)C(C)N(CC)CC]

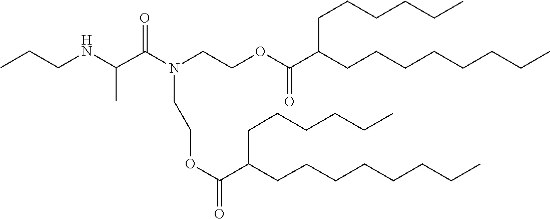 [SMILES: CCCCCCCCC(CCCCCC)C(=O)OCCN(CCOC(=O)C(CCCCCC)CCCCCCCC)C(=O)C(C)NCCC]

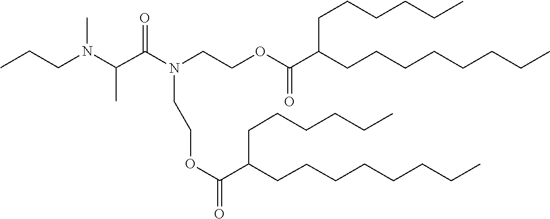 [SMILES: CCCCCCCCC(CCCCCC)C(=O)OCCN(CCOC(=O)C(CCCCCC)CCCCCCCC)C(=O)C(C)N(C)CCC]

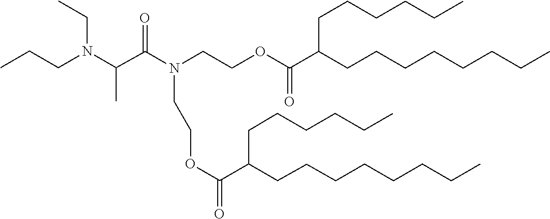 [SMILES: CCCCCCCCC(CCCCCC)C(=O)OCCN(CCOC(=O)C(CCCCCC)CCCCCCCC)C(=O)C(C)N(CC)CCC]

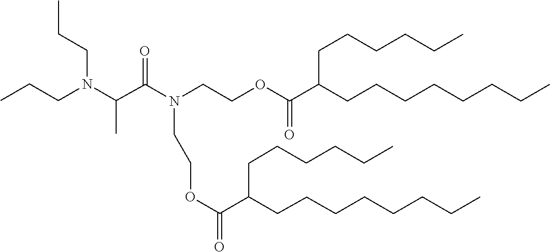 [SMILES: CCCCCCCCC(CCCCCC)C(=O)OCCN(CCOC(=O)C(CCCCCC)CCCCCCCC)C(=O)C(C)N(CCC)CCC]

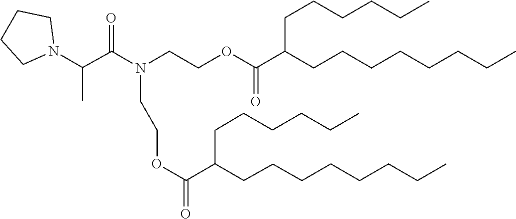 [SMILES: CCCCCCCCC(CCCCCC)C(=O)OCCN(CCOC(=O)C(CCCCCC)CCCCCCCC)C(=O)C(C)N1CCCC1]

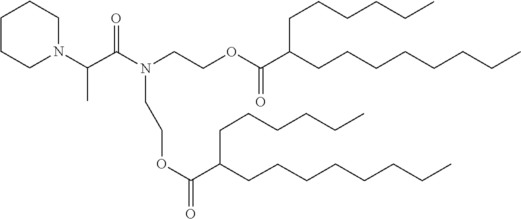 [SMILES: CCCCCCCCC(CCCCCC)C(=O)OCCN(CCOC(=O)C(CCCCCC)CCCCCCCC)C(=O)C(C)N1CCCCC1]

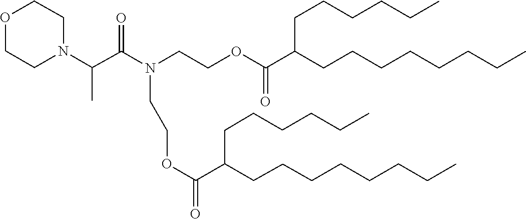 [SMILES: CCCCCCCCC(CCCCCC)C(=O)OCCN(CCOC(=O)C(CCCCCC)CCCCCCCC)C(=O)C(C)N1CCOCC1]

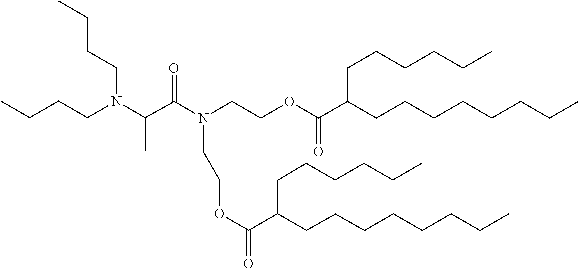 [SMILES: CCCCCCCCC(CCCCCC)C(=O)OCCN(CCOC(=O)C(CCCCCC)CCCCCCCC)C(=O)C(C)N(CCCC)CCCC]

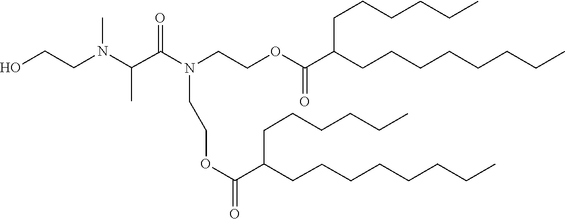 [SMILES: CCCCCCCCC(CCCCCC)C(=O)OCCN(CCOC(=O)C(CCCCCC)CCCCCCCC)C(=O)C(C)N(C)CCO]

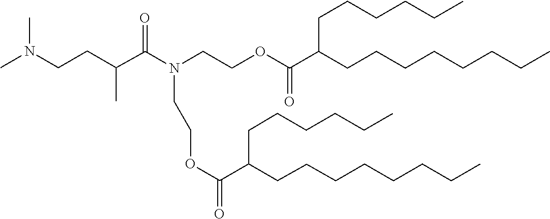 [SMILES: CCCCCCCCC(CCCCCC)C(=O)OCCN(CCOC(=O)C(CCCCCC)CCCCCCCC)C(=O)C(C)CCN(C)C]

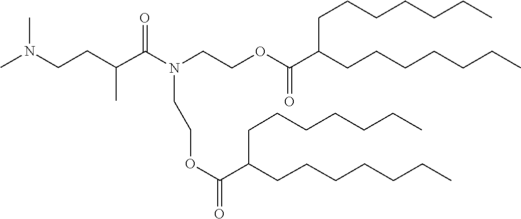 [SMILES: CCCCCCCC(CCCCCCC)C(=O)OCCN(CCOC(=O)C(CCCCCCC)CCCCCCC)C(=O)C(C)CCN(C)C]

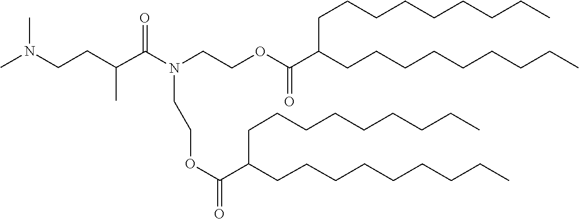 [SMILES: CCCCCCCCCC(CCCCCCCCC)C(=O)OCCN(CCOC(=O)C(CCCCCCCCC)CCCCCCCCC)C(=O)C(C)CCN(C)C]

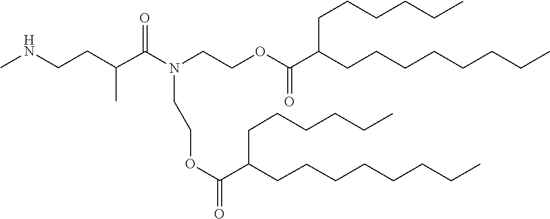 [SMILES: CCCCCCCCC(CCCCCC)C(=O)OCCN(CCOC(=O)C(CCCCCC)CCCCCCCC)C(=O)C(C)CCNC]

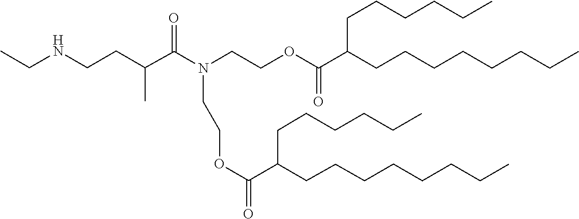 [SMILES: CCCCCCCCC(CCCCCC)C(=O)OCCN(CCOC(=O)C(CCCCCC)CCCCCCCC)C(=O)C(C)CCNCC]

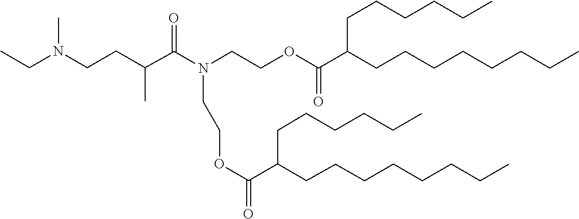 [SMILES: CCCCCCCCC(CCCCCC)C(=O)OCCN(CCOC(=O)C(CCCCCC)CCCCCCCC)C(=O)C(C)CCN(C)CC]

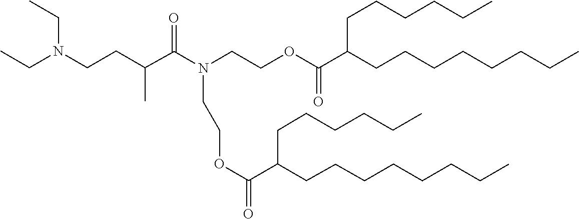 [SMILES: CCCCCCCCC(CCCCCC)C(=O)OCCN(CCOC(=O)C(CCCCCC)CCCCCCCC)C(=O)C(C)CCN(CC)CC]

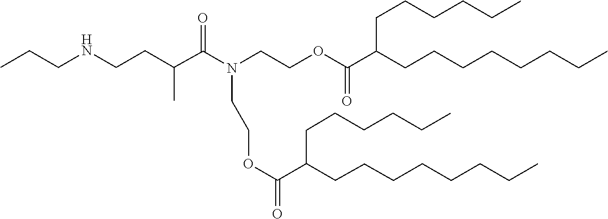 [SMILES: CCCCCCCCC(CCCCCC)C(=O)OCCN(CCOC(=O)C(CCCCCC)CCCCCCCC)C(=O)C(C)CCNCCC]

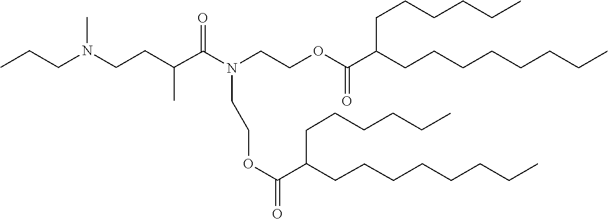 [SMILES: CCCCCCCCC(CCCCCC)C(=O)OCCN(CCOC(=O)C(CCCCCC)CCCCCCCC)C(=O)C(C)CCN(C)CCC]

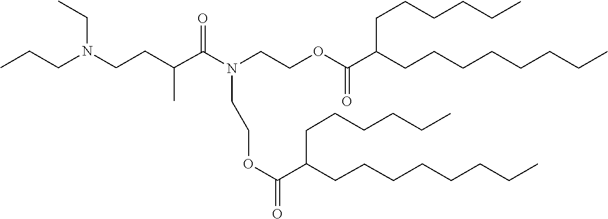 [SMILES: CCCCCCCCC(CCCCCC)C(=O)OCCN(CCOC(=O)C(CCCCCC)CCCCCCCC)C(=O)C(C)CCN(CC)CCC]

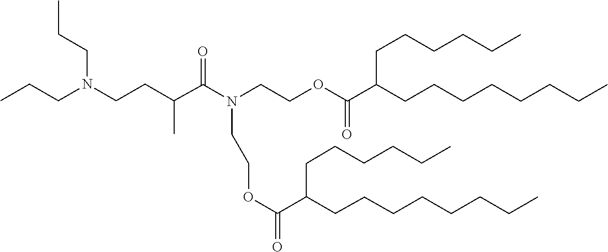 [SMILES: CCCCCCCCC(CCCCCC)C(=O)OCCN(CCOC(=O)C(CCCCCC)CCCCCCCC)C(=O)C(C)CCN(CCC)CCC]

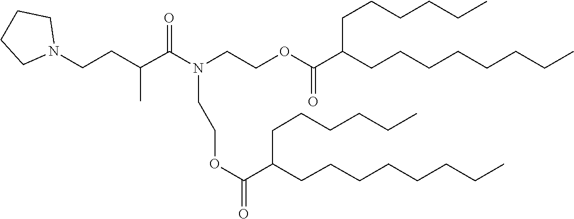 [SMILES: CCCCCCCCC(CCCCCC)C(=O)OCCN(CCOC(=O)C(CCCCCC)CCCCCCCC)C(=O)C(C)CCN1CCCC1]

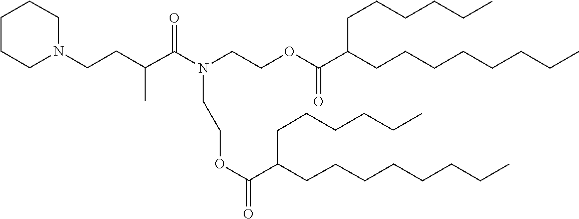 [SMILES: CCCCCCCCC(CCCCCC)C(=O)OCCN(CCOC(=O)C(CCCCCC)CCCCCCCC)C(=O)C(C)CCN1CCCCC1]

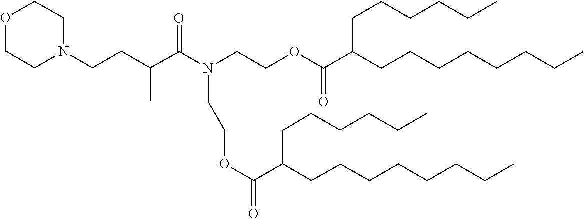 [SMILES: CCCCCCCCC(CCCCCC)C(=O)OCCN(CCOC(=O)C(CCCCCC)CCCCCCCC)C(=O)C(C)CCN1CCOCC1]

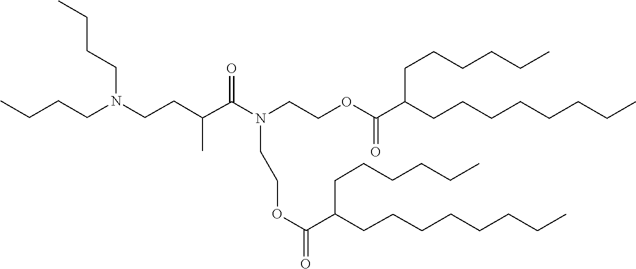 [SMILES: CCCCCCCCC(CCCCCC)C(=O)OCCN(CCOC(=O)C(CCCCCC)CCCCCCCC)C(=O)C(C)CCN(CCCC)CCCC]

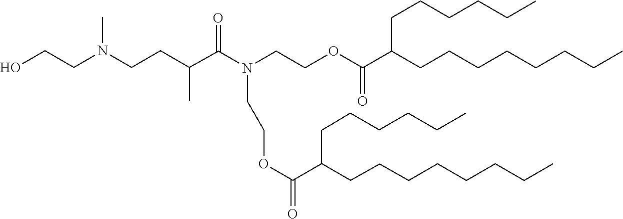 [SMILES: CCCCCCCCC(CCCCCC)C(=O)OCCN(CCOC(=O)C(CCCCCC)CCCCCCCC)C(=O)C(C)CCN(C)CCO]

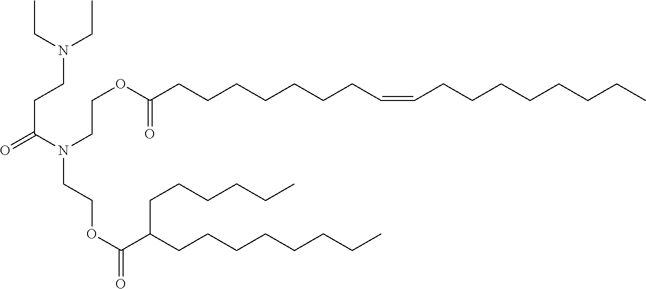 [SMILES: CCCCCCCC/C=C\CCCCCCCC(=O)OCCN(CCOC(=O)C(CCCCCC)CCCCCCCC)C(=O)CCN(CC)CC]

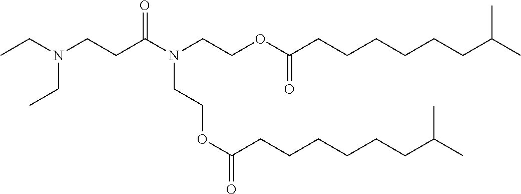 [SMILES: CCN(CC)CCC(=O)N(CCOC(=O)CCCCCCC(C)C)CCOC(=O)CCCCCCC(C)C]

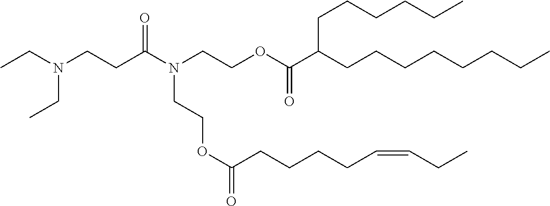 [SMILES: CC/C=C\CCCCC(=O)OCCN(CCOC(=O)C(CCCCCC)CCCCCCCC)C(=O)CCN(CC)CC]

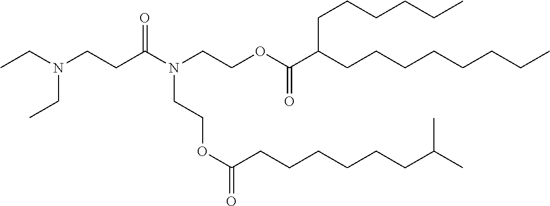 [SMILES: CCCCCCCCC(CCCCCC)C(=O)OCCN(CCOC(=O)CCCCCCC(C)C)C(=O)CCN(CC)CC]

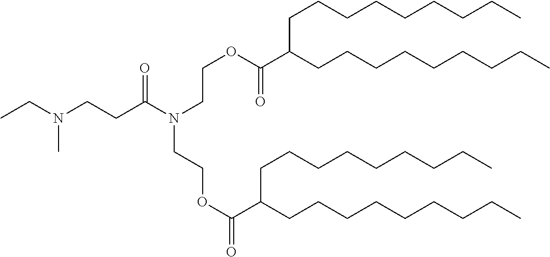 [SMILES: CCCCCCCCCC(CCCCCCCCC)C(=O)OCCN(CCOC(=O)C(CCCCCCCCC)CCCCCCCCC)C(=O)CCN(C)CC]

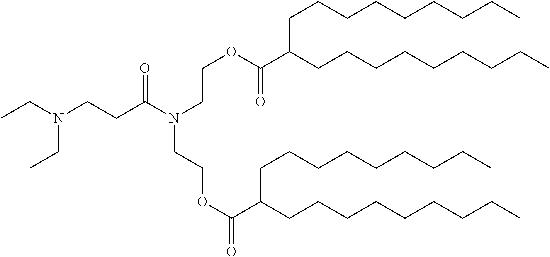 [SMILES: CCCCCCCCCC(CCCCCCCCC)C(=O)OCCN(CCOC(=O)C(CCCCCCCCC)CCCCCCCCC)C(=O)CCN(CC)CC]

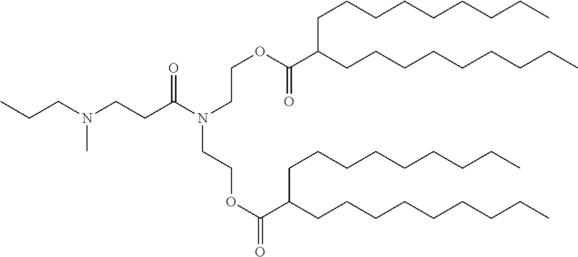 [SMILES: CCCCCCCCCC(CCCCCCCCC)C(=O)OCCN(CCOC(=O)C(CCCCCCCCC)CCCCCCCCC)C(=O)CCN(C)CCC]

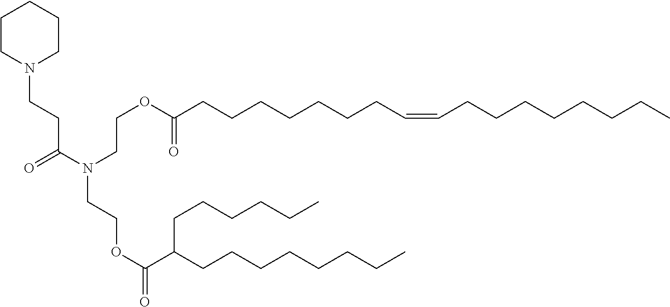 [SMILES: CCCCCCCC/C=C\CCCCCCCC(=O)OCCN(CCOC(=O)C(CCCCCC)CCCCCCCC)C(=O)CCN1CCCCC1]

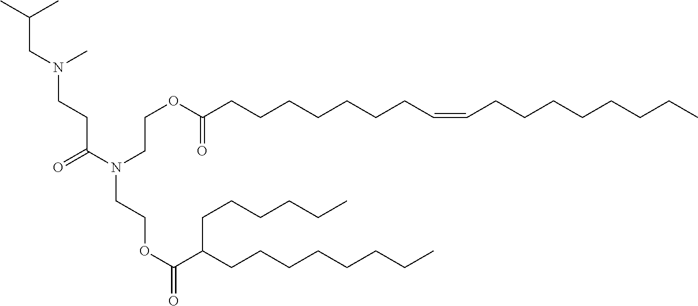 [SMILES: CCCCCCCC/C=C\CCCCCCCC(=O)OCCN(CCOC(=O)C(CCCCCC)CCCCCCCC)C(=O)CCN(C)CC(C)C]

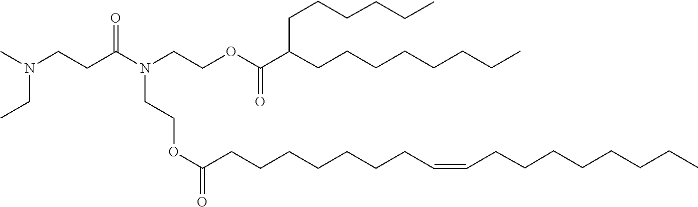 [SMILES: CCCCCCCC/C=C\CCCCCCCC(=O)OCCN(CCOC(=O)C(CCCCCC)CCCCCCCC)C(=O)CCN(C)CC]

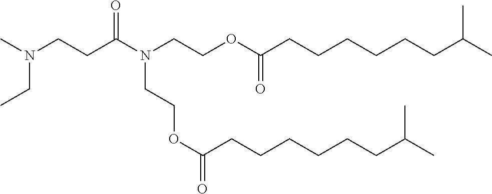 [SMILES: CCN(C)CCC(=O)N(CCOC(=O)CCCCCCC(C)C)CCOC(=O)CCCCCCC(C)C]

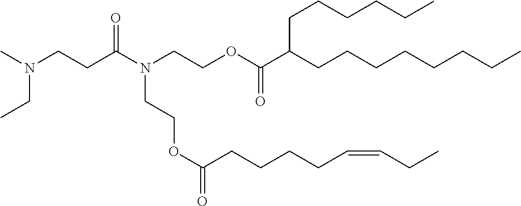 [SMILES: CC/C=C\CCCCC(=O)OCCN(CCOC(=O)C(CCCCCC)CCCCCCCC)C(=O)CCN(C)CC]

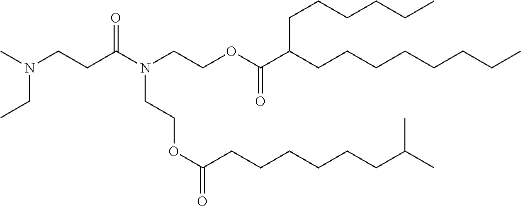 [SMILES: CCCCCCCCC(CCCCCC)C(=O)OCCN(CCOC(=O)CCCCCCC(C)C)C(=O)CCN(C)CC]

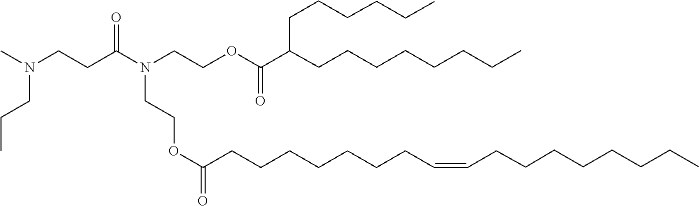 [SMILES: CCCCCCCC/C=C\CCCCCCCC(=O)OCCN(CCOC(=O)C(CCCCCC)CCCCCCCC)C(=O)CCN(C)CCC]

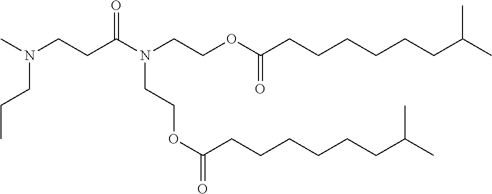 [SMILES: CCCN(C)CCC(=O)N(CCOC(=O)CCCCCCC(C)C)CCOC(=O)CCCCCCC(C)C]

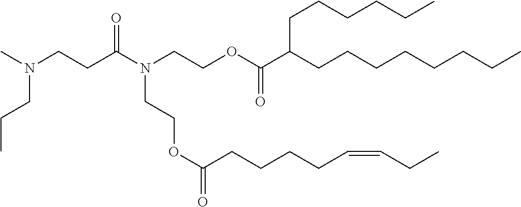 [SMILES: CC/C=C\CCCCC(=O)OCCN(CCOC(=O)C(CCCCCC)CCCCCCCC)C(=O)CCN(C)CCC]

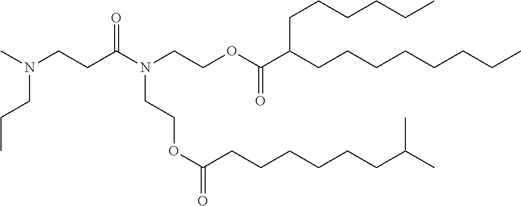 [SMILES: CCCCCCCCC(CCCCCC)C(=O)OCCN(CCOC(=O)CCCCCCC(C)C)C(=O)CCN(C)CCC]

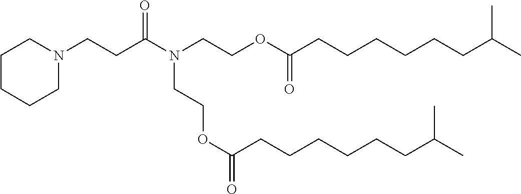 [SMILES: CC(C)CCCCCCC(=O)OCCN(CCOC(=O)CCCCCCC(C)C)C(=O)CCN1CCCCC1]

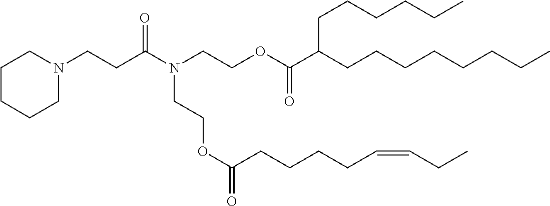 [SMILES: CC/C=C\CCCCC(=O)OCCN(CCOC(=O)C(CCCCCC)CCCCCCCC)C(=O)CCN1CCCCC1]

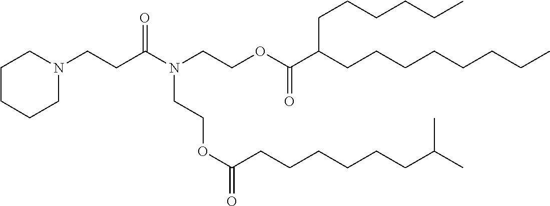 [SMILES: CCCCCCCCC(CCCCCC)C(=O)OCCN(CCOC(=O)CCCCCCC(C)C)C(=O)CCN1CCCCC1]

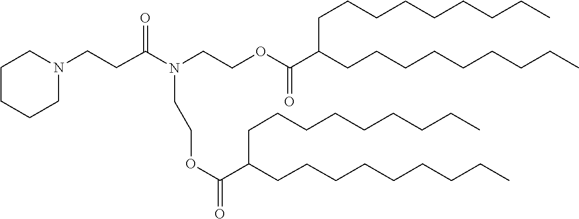 [SMILES: CCCCCCCCCC(CCCCCCCCC)C(=O)OCCN(CCOC(=O)C(CCCCCCCCC)CCCCCCCCC)C(=O)CCN1CCCCC1]

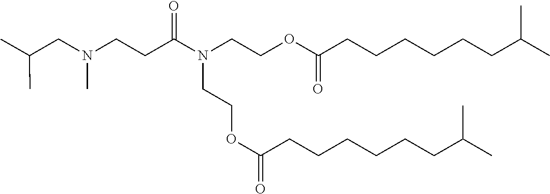 [SMILES: CC(C)CCCCCCC(=O)OCCN(CCOC(=O)CCCCCCC(C)C)C(=O)CCN(C)CC(C)C]

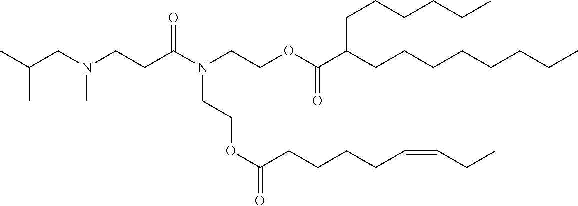 [SMILES: CC/C=C\CCCCC(=O)OCCN(CCOC(=O)C(CCCCCC)CCCCCCCC)C(=O)CCN(C)CC(C)C]

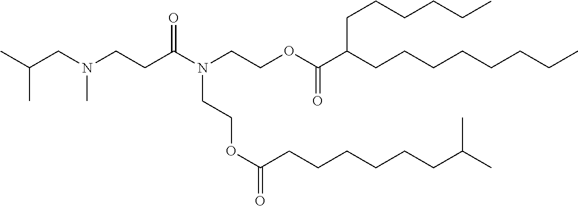 [SMILES: CCCCCCCCC(CCCCCC)C(=O)OCCN(CCOC(=O)CCCCCCC(C)C)C(=O)CCN(C)CC(C)C]

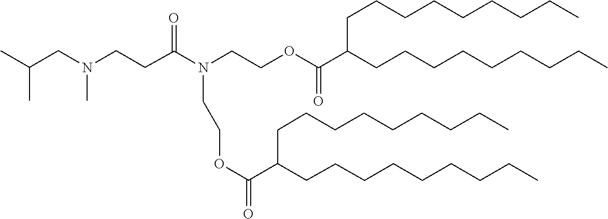 [SMILES: CCCCCCCCCC(CCCCCCCCC)C(=O)OCCN(CCOC(=O)C(CCCCCCCCC)CCCCCCCCC)C(=O)CCN(C)CC(C)C]

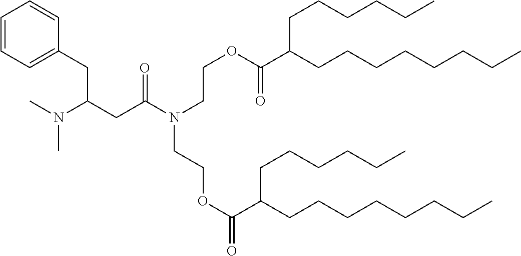 [SMILES: CCCCCCCCC(CCCCCC)C(=O)OCCN(CCOC(=O)C(CCCCCC)CCCCCCCC)C(=O)CC(Cc1ccccc1)N(C)C]

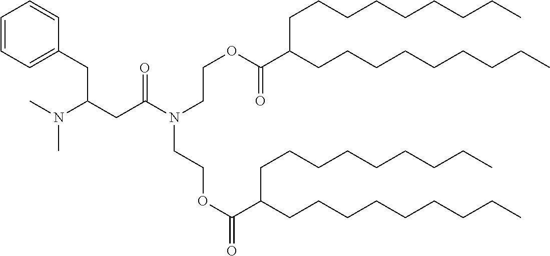 [SMILES: CCCCCCCCCC(CCCCCCCCC)C(=O)OCCN(CCOC(=O)C(CCCCCCCCC)CCCCCCCCC)C(=O)CC(Cc1ccccc1)N(C)C]

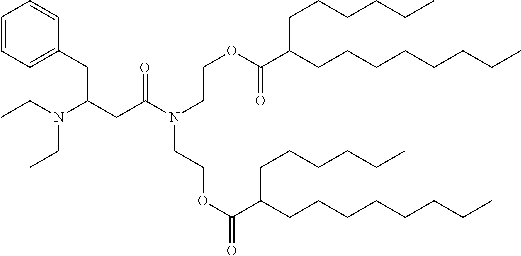 [SMILES: CCCCCCCCC(CCCCCC)C(=O)OCCN(CCOC(=O)C(CCCCCC)CCCCCCCC)C(=O)CC(Cc1ccccc1)N(CC)CC]

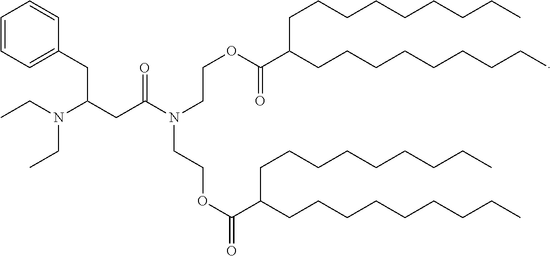

Provided herein is a lipidoid having the structure of formula (II):
or a salt thereof;

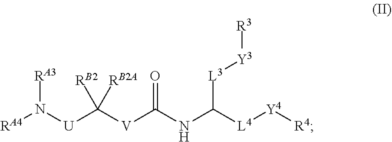

wherein:
R^{A3} and R^{A4} are each independently H, alkyl, or hydroxyalkyl;
or wherein R^{A3} and R^{A4}, taken together with the intervening nitrogen, form an optionally substituted heterocycloalkyl ring;
R^{B2} and R^{B2A} are each independently H, optionally substituted alkyl, alkenyl, cycloalkyl, arylalkyl, heteroarylalkyl, hydroxyalkyl, alkoxyalkyl, aminoalkyl, or thioalkyl;
or wherein R^{B2} and R^{B2A}, taken together with the intervening carbon, form an optionally substituted cycloalkyl or heterocycloalkyl ring;
or R^{A3} and R^{B2} taken together with the intervening N—CH atoms to which they are attached form an N-containing heterocyclic ring;
L³ and L⁴ are each independently —CH₂—, —CH₂CH₂—, —(CH₂)₃—, —(CH₂)₄—, —CH(CH₃)CH₂—, or —CH₂CH(CH₃)—;
V is a bond, —CH₂— or —CH₂CH₂—;
U is a bond, —CH₂— or —CH₂CH₂—;
Y³ and Y⁴ are each independently —O(C═O)—, —S(C═O)—, or —O(C═O)O—; and
R³ and R⁴ are each independently linear (C₈-C₃₀)alkyl, branched (C₈-C₃₀)alkyl, linear (C₈-C₃₀)alkenyl, branched (C₈-C₃₀)alkenyl, linear (C₈-C₃₀)alkynyl, or branched (C₈-C₃₀)alkynyl; wherein a branched (C₈-C₃₀)alkyl, a branched (C₈-C₃₀)alkenyl, or a branched (C₈-C₃₀)alkynyl may comprise more than one branch point, and a linear or branched (C₈-C₃₀)alkenyl may comprise more than one carbon-carbon double bond, and a linear or branched (C₈-C₃₀)alkynyl may comprise more than one carbon-carbon triple bond.

In some embodiments, R^{A3} and R^{A4} are the same. In certain exemplary embodiments, RA³ and RA⁴ are each H.

In certain embodiments, R^{A3} and R^{A4} are different. In certain such embodiments, R^{A3}is hydroxyalkyl; and R^{A4} is H.

In certain embodiments, R^{A3} is —CH₂CH₂OH.

In further embodiments, R^{B2} is H, optionally substituted alkyl, arylalkyl, heteroarylalkyl, hydroxyalkyl, or aminoalkyl.

In certain embodiments, wherein R^{B2} is H, methyl, —CH₂Ph, —CH₂OH, —(CH₂)₄NH₂, or —CH₂(imidazolyl).

In certain embodiments, R^{B2} is methyl.

In yet other embodiments, R^{B2A} is hydrogen.

In certain embodiments, at least one of U and V is a bond. In certain such embodiments, U and V are both a bond.

In certain such embodiments, the lipidoid has the structure of formula (IIa):

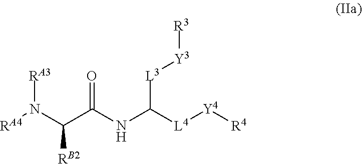

or a salt thereof;
wherein the lipidoid has the absolute stereochemistry shown.

In alternative such embodiments, the lipidoid has the structure of formula (IIb):

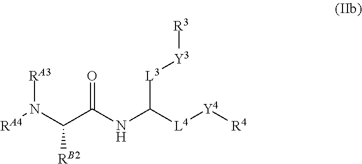

or a salt thereof;
wherein the lipidoid has the absolute stereochemistry shown.

In certain embodiments, V is —CH₂—; and U is a bond.

In certain embodiments, the present disclosure provides a lipidoid having the structure of formula (IIc):

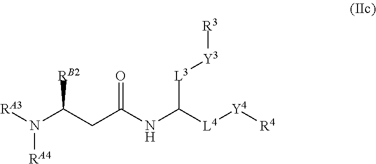

or a salt thereof;
wherein the lipidoid has the absolute stereochemistry shown.

In other alternative embodiments, the lipidoid has the structure of formula (IId):

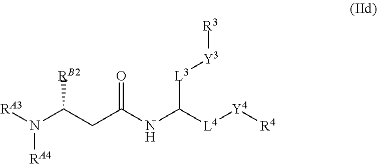

or a salt thereof;
wherein the lipidoid has the absolute stereochemistry shown.

In certain embodiments, V is —CH₂CH₂—; and U is a bond.

In certain embodiments, the present disclosure provides a lipidoid having the structure of formula (IIe):

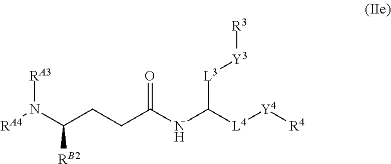

or a salt thereof;
wherein the lipidoid has the absolute stereochemistry shown.

In certain alternative embodiments, the lipidoid has the structure of formula (IIf):

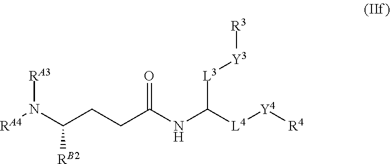

or a salt thereof;
wherein the lipidoid has the absolute stereochemistry shown.

In certain embodiments, U is a bond.

In certain embodiments, Y³ and Y⁴ are each —O(C═O)—

In certain embodiments, L³ and L⁴ each represent —CH₂—.

In certain embodiments, R³ and R⁴ are each independently linear (C₈-C₂₄)alkyl, branched (C₈-C₂₄)alkyl, linear (C₈-C₂₄)alkenyl, or branched (C₈-C₂₄)alkenyl.

In certain alternative embodiments, R³ and R⁴ are each independently linear (C₈-C₂₄)alkyl or linear (C₈-C₂₄)alkenyl.

In some embodiments, R³ and R⁴ are each independently linear (C₈-C₂₄)alkyl.

In other embodiments, R³ and R⁴ are each independently linear (C₉-C₁₇)alkyl.

In certain embodiments, R³ and R⁴ are each independently a linear (C₈-C₂₄)alkenyl.

In yet other embodiments, R³ and R⁴ are each independently a linear (C₁₇)alkenyl.

In certain other embodiments, R³ and R⁴ are each independently branched (C₈-C₂₄)alkyl.

In some embodiments, R³ and R⁴ are each independently a branched (C₁₅-C₁₉)alkyl.

In certain preferred embodiments, the salt is a pharmaceutically acceptable salt.

Representative Series 400 lipidoids include those in the table below:

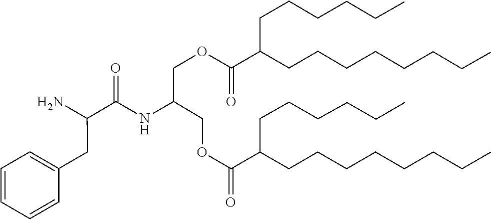 [SMILES: CCCCCCCCC(CCCCCC)C(=O)OCC(COC(=O)C(CCCCCC)CCCCCCCC)NC(=O)C(N)Cc1ccccc1]

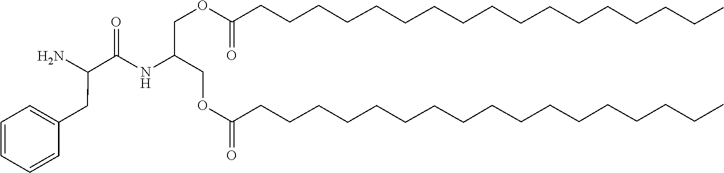 [SMILES: CCCCCCCCCCCCCCCCCC(=O)OCC(COC(=O)CCCCCCCCCCCCCCCCC)NC(=O)C(N)Cc1ccccc1]

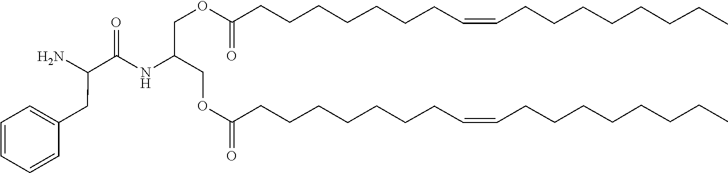 [SMILES: CCCCCCCC/C=C\CCCCCCCC(=O)OCC(COC(=O)CCCCCCC/C=C\CCCCCCCC)NC(=O)C(N)Cc1ccccc1]

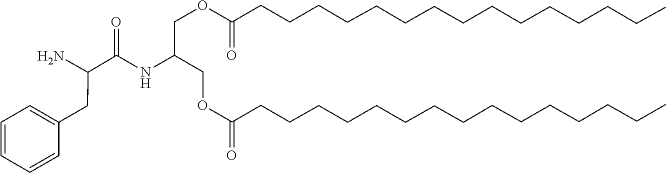 [SMILES: CCCCCCCCCCCCCCCC(=O)OCC(COC(=O)CCCCCCCCCCCCCCC)NC(=O)C(N)Cc1ccccc1]

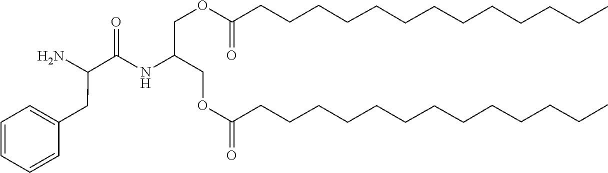 [SMILES: CCCCCCCCCCCCCC(=O)OCC(COC(=O)CCCCCCCCCCCCC)NC(=O)C(N)Cc1ccccc1]

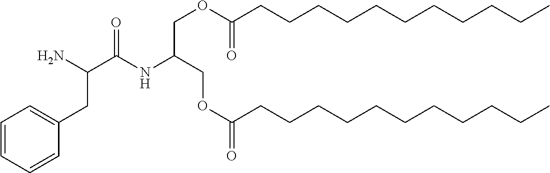 [SMILES: CCCCCCCCCCCC(=O)OCC(COC(=O)CCCCCCCCCCC)NC(=O)C(N)Cc1ccccc1]

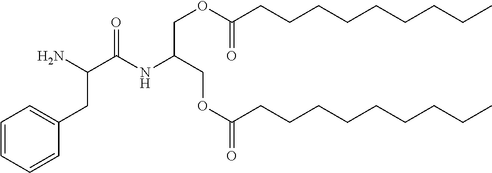 [SMILES: CCCCCCCCCC(=O)OCC(COC(=O)CCCCCCCCC)NC(=O)C(N)Cc1ccccc1]

Additional representative Series 300 lipidoids include those in the table below:

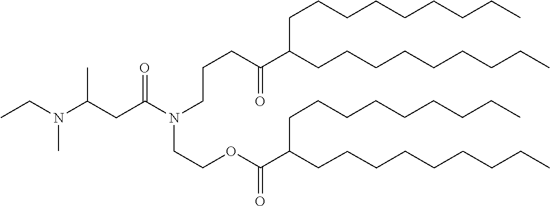 [SMILES: CCCCCCCCCC(CCCCCCCCC)C(=O)CCCN(CCOC(=O)C(CCCCCCCCC)CCCCCCCCC)C(=O)CC(C)N(C)CC]

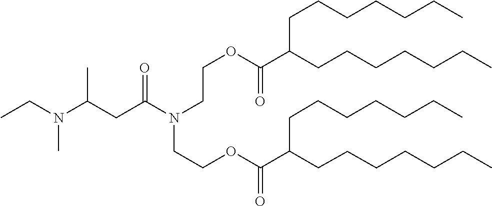 [SMILES: CCCCCCCC(CCCCCCC)C(=O)OCCN(CCOC(=O)C(CCCCCCC)CCCCCCC)C(=O)CC(C)N(C)CC]

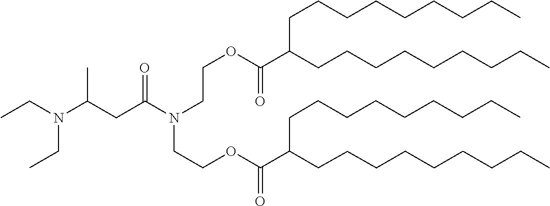 [SMILES: CCCCCCCCCC(CCCCCCCCC)C(=O)OCCN(CCOC(=O)C(CCCCCCCCC)CCCCCCCCC)C(=O)CC(C)N(CC)CC]

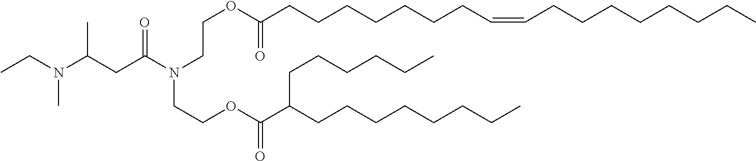 [SMILES: CCCCCCCC/C=C\CCCCCCCC(=O)OCCN(CCOC(=O)C(CCCCCC)CCCCCCCC)C(=O)CC(C)N(C)CC]

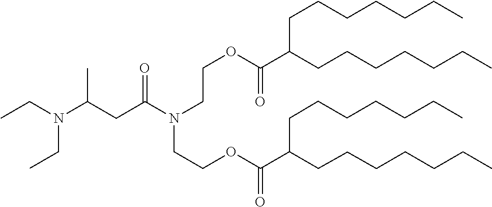 [SMILES: CCCCCCCC(CCCCCCC)C(=O)OCCN(CCOC(=O)C(CCCCCCC)CCCCCCC)C(=O)CC(C)N(CC)CC]

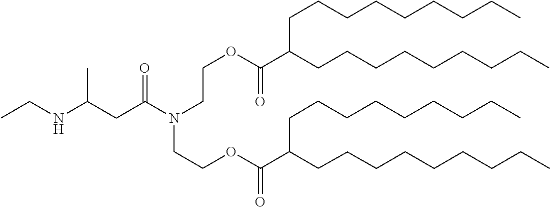 [SMILES: CCCCCCCCCC(CCCCCCCCC)C(=O)OCCN(CCOC(=O)C(CCCCCCCCC)CCCCCCCCC)C(=O)CC(C)NCC]

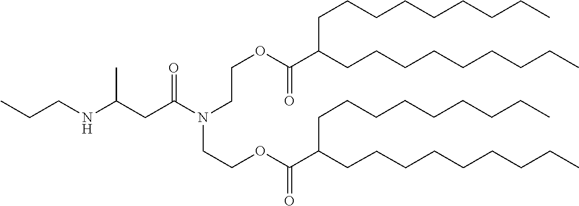 [SMILES: CCCCCCCCCC(CCCCCCCCC)C(=O)OCCN(CCOC(=O)C(CCCCCCCCC)CCCCCCCCC)C(=O)CC(C)NCCC]

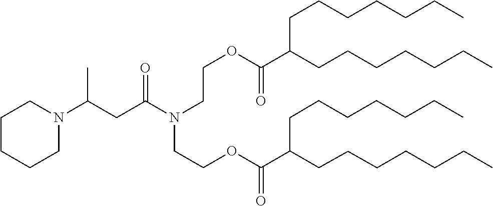 [SMILES: CCCCCCCC(CCCCCCC)C(=O)OCCN(CCOC(=O)C(CCCCCCC)CCCCCCC)C(=O)CC(C)N1CCCCC1]

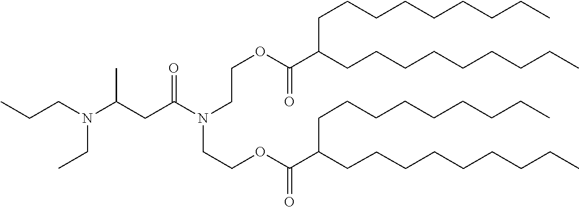 [SMILES: CCCCCCCCCC(CCCCCCCCC)C(=O)OCCN(CCOC(=O)C(CCCCCCCCC)CCCCCCCCC)C(=O)CC(C)N(CC)CCC]

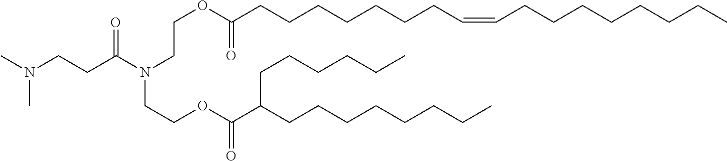 [SMILES: CCCCCCCC/C=C\CCCCCCCC(=O)OCCN(CCOC(=O)C(CCCCCC)CCCCCCCC)C(=O)CCN(C)C]

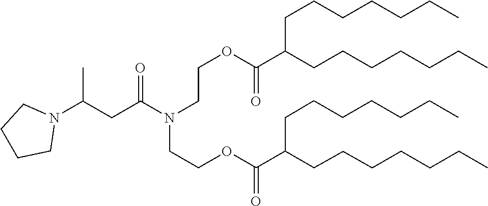 [SMILES: CCCCCCCC(CCCCCCC)C(=O)OCCN(CCOC(=O)C(CCCCCCC)CCCCCCC)C(=O)CC(C)N1CCCC1]

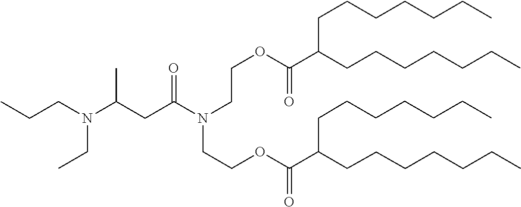 [SMILES: CCCCCCCC(CCCCCCC)C(=O)OCCN(CCOC(=O)C(CCCCCCC)CCCCCCC)C(=O)CC(C)N(CC)CCC]

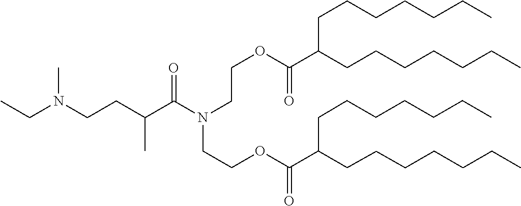 [SMILES: CCCCCCCC(CCCCCCC)C(=O)OCCN(CCOC(=O)C(CCCCCCC)CCCCCCC)C(=O)C(C)CCN(C)CC]

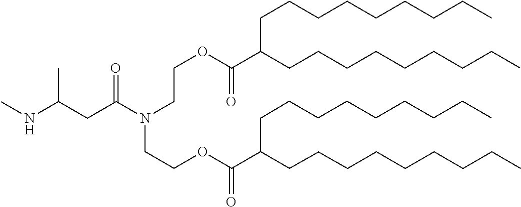 [SMILES: CCCCCCCCCC(CCCCCCCCC)C(=O)OCCN(CCOC(=O)C(CCCCCCCCC)CCCCCCCCC)C(=O)CC(C)NC]

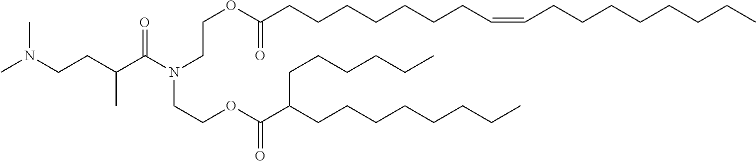 [SMILES: CCCCCCCC/C=C\CCCCCCCC(=O)OCCN(CCOC(=O)C(CCCCCC)CCCCCCCC)C(=O)C(C)CCN(C)C]

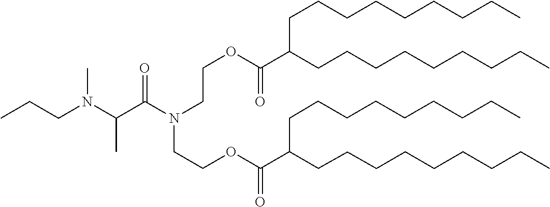 [SMILES: CCCCCCCCCC(CCCCCCCCC)C(=O)OCCN(CCOC(=O)C(CCCCCCCCC)CCCCCCCCC)C(=O)C(C)N(C)CCC]

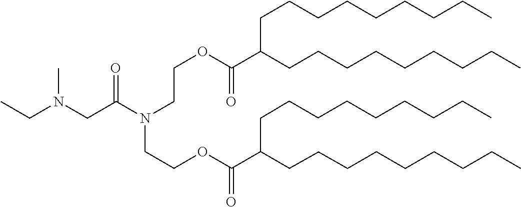 [SMILES: CCCCCCCCCC(CCCCCCCCC)C(=O)OCCN(CCOC(=O)C(CCCCCCCCC)CCCCCCCCC)C(=O)CN(C)CC]

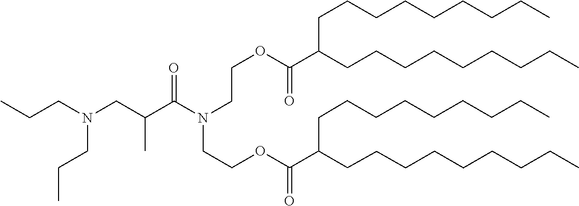 [SMILES: CCCCCCCCCC(CCCCCCCCC)C(=O)OCCN(CCOC(=O)C(CCCCCCCCC)CCCCCCCCC)C(=O)C(C)CN(CCC)CCC]

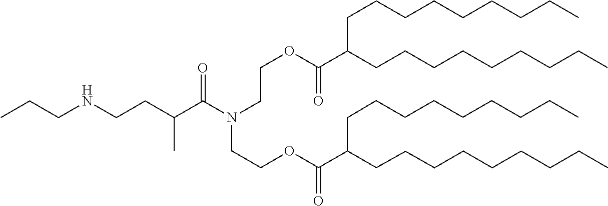 [SMILES: CCCCCCCCCC(CCCCCCCCC)C(=O)OCCN(CCOC(=O)C(CCCCCCCCC)CCCCCCCCC)C(=O)C(C)CCNCCC]

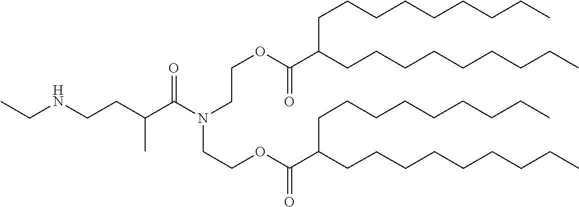 [SMILES: CCCCCCCCCC(CCCCCCCCC)C(=O)OCCN(CCOC(=O)C(CCCCCCCCC)CCCCCCCCC)C(=O)C(C)CCNCC]

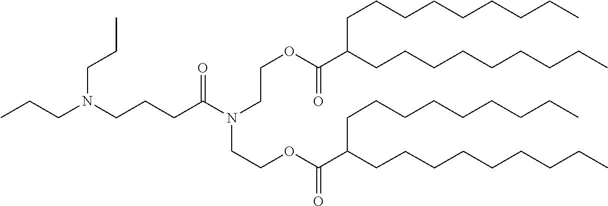 [SMILES: CCCCCCCCCC(CCCCCCCCC)C(=O)OCCN(CCOC(=O)C(CCCCCCCCC)CCCCCCCCC)C(=O)CCCN(CCC)CCC]

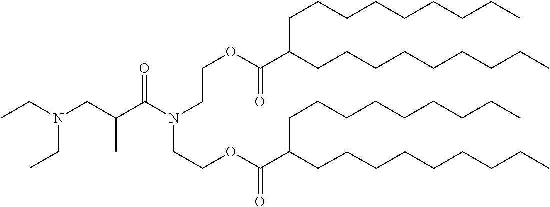 [SMILES: CCCCCCCCCC(CCCCCCCCC)C(=O)OCCN(CCOC(=O)C(CCCCCCCCC)CCCCCCCCC)C(=O)C(C)CN(CC)CC]

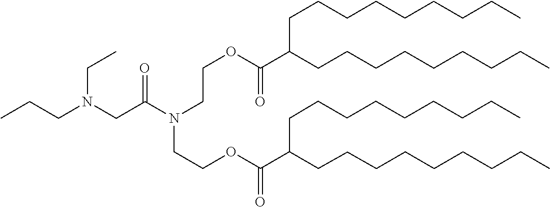 [SMILES: CCCCCCCCCC(CCCCCCCCC)C(=O)OCCN(CCOC(=O)C(CCCCCCCCC)CCCCCCCCC)C(=O)CN(CC)CCC]

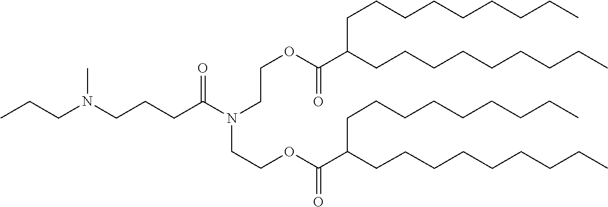 [SMILES: CCCCCCCCCC(CCCCCCCCC)C(=O)OCCN(CCOC(=O)C(CCCCCCCCC)CCCCCCCCC)C(=O)CCCN(C)CCC]

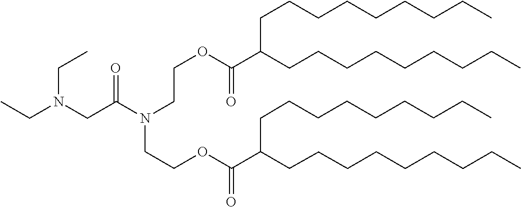 [SMILES: CCCCCCCCCC(CCCCCCCCC)C(=O)OCCN(CCOC(=O)C(CCCCCCCCC)CCCCCCCCC)C(=O)CN(CC)CC]

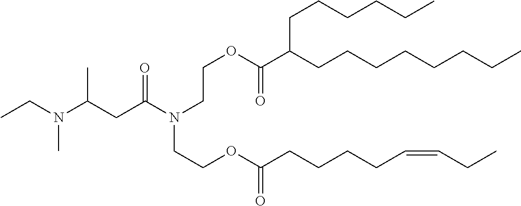 [SMILES: CC/C=C\CCCCC(=O)OCCN(CCOC(=O)C(CCCCCC)CCCCCCCC)C(=O)CC(C)N(C)CC]

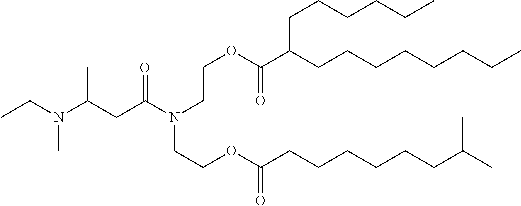 [SMILES: CCCCCCCCC(CCCCCC)C(=O)OCCN(CCOC(=O)CCCCCCC(C)C)C(=O)CC(C)N(C)CC]

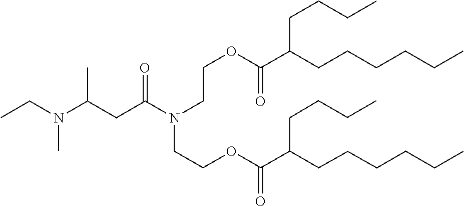 [SMILES: CCCCCCC(CCCC)C(=O)OCCN(CCOC(=O)C(CCCC)CCCCCC)C(=O)CC(C)N(C)CC]

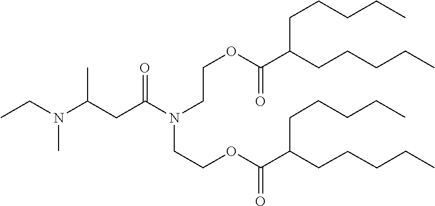 [SMILES: CCCCCC(CCCCC)C(=O)OCCN(CCOC(=O)C(CCCCC)CCCCC)C(=O)CC(C)N(C)CC]

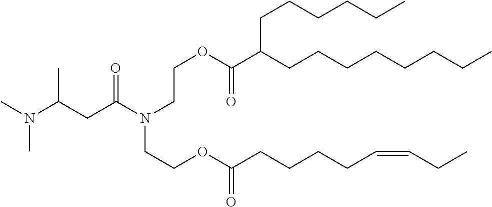 [SMILES: CC/C=C\CCCCC(=O)OCCN(CCOC(=O)C(CCCCCC)CCCCCCCC)C(=O)CC(C)N(C)C]

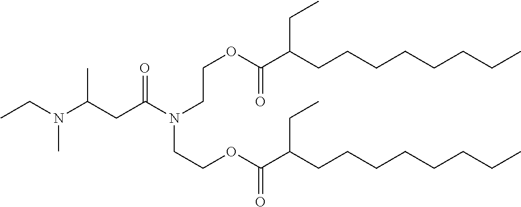 [SMILES: CCCCCCCCC(CC)C(=O)OCCN(CCOC(=O)C(CC)CCCCCCCC)C(=O)CC(C)N(C)CC]

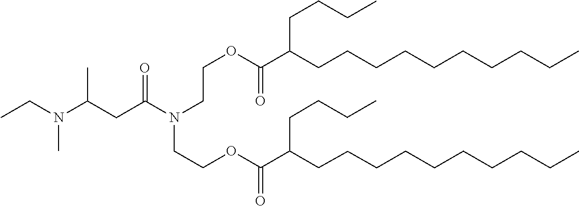 [SMILES: CCCCCCCCCCC(CCCC)C(=O)OCCN(CCOC(=O)C(CCCC)CCCCCCCCCC)C(=O)CC(C)N(C)CC]

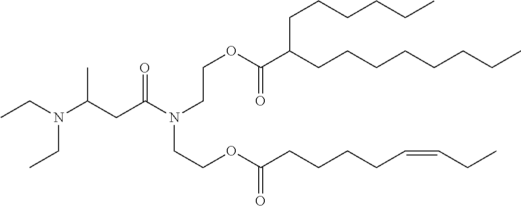 [SMILES: CC/C=C\CCCCC(=O)OCCN(CCOC(=O)C(CCCCCC)CCCCCCCC)C(=O)CC(C)N(CC)CC]

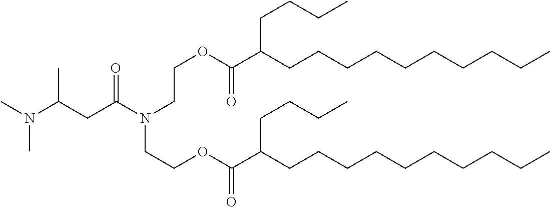 [SMILES: CCCCCCCCCCC(CCCC)C(=O)OCCN(CCOC(=O)C(CCCC)CCCCCCCCCC)C(=O)CC(C)N(C)C]

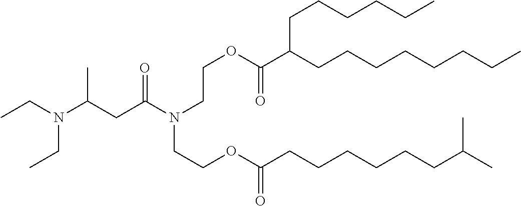 [SMILES: CCCCCCCCC(CCCCCC)C(=O)OCCN(CCOC(=O)CCCCCCC(C)C)C(=O)CC(C)N(CC)CC]

Also provided herein is a lipidoid having the structure of formula (Ix):
or a salt thereof;

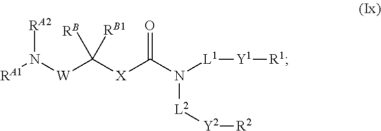

wherein:
R^{A1} and R^{A2} are each independently H, methyl, ethyl, or propyl;
or wherein R^{A1} and R^{A2}, taken together with the intervening nitrogen, form a 5- or 6-membered heterocyclic ring;
R^{B} and R^{B1} are each independently H or methyl;
L¹ and L² are each —CH₂CH₂—;
X is a bond;
W is a bond or —CH₂—;
Y¹ and Y² are each independently —O(C═O)—;
R¹ and R² are each independently branched (C₁₄-C₂₀)alkyl; wherein a branched (C₁₄-C₂₀)alkyl may comprise more than one branch point;
provided that
if X and W are both a bond, and R^{B} is H, then R^{A1} and R^{A2} are not both methyl.

Also provided herein is a lipidoid having the structure of formula (Iy): or a salt thereof;

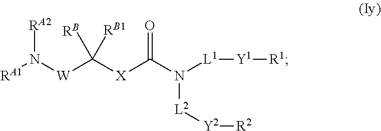

wherein:
R^{A1} and R^{A2} are each independently H, methyl, ethyl, or propyl;
or wherein R^{A1} and R^{A2}, taken together with the intervening nitrogen, form a 5- or 6-membered heterocyclic ring;
R^{B} and R^{B1} are each independently H or methyl;
L¹ and L² are each —CH₂CH₂—;
X is a bond or —CH₂—;
W is a bond or —CH₂—;
Y¹ and Y² are each independently —O(C═O)—; and
R¹ and R² are each independently branched (C₁₄-C₂₀)alkyl; wherein a branched (C₁₄-C₂₀)alkyl may comprise more than one branch point;
provided that
if X and W are both a bond, and R^{B} is H, then R^{A1} and R^{A2} are not both methyl.

In certain embodiments, L¹ and L² are the same.

In other embodiments, L¹ and L² are different.

In certain embodiments, Y¹ and Y² are the same.

In certain alternative embodiments, Y¹ and Y² are different.

In certain embodiments, R¹ is

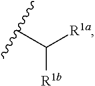

and R^{1a} and R^{1b} are each independently (C₁-C₂₂)alkyl, wherein R¹ contains from 8 to 24 total carbon atoms.

In certain embodiments, R^{1a} and R^{1b} are the same.

In certain embodiments, R^{1a} and R^{1b} are different.

In certain embodiments, R² is,

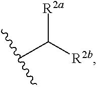

and R^{2a} and R^{2b} are each independently (C₁-C₂₂)alkyl, wherein R² contains from 8 to 24 total carbon atoms.

In certain embodiments, R^{2a} and R^{2b} are the same

In certain embodiments, R^{2a} and R^{2b} are different.

Certain embodiments of the present disclosure provide a lipidoid having the structure of Formula (Iz):

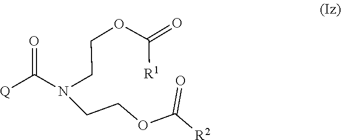

or a salt thereof,
wherein Q is

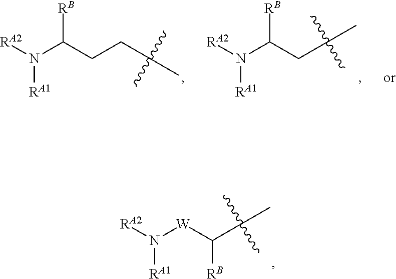

preferably

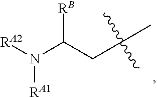

In certain embodiments, Q is selected from:

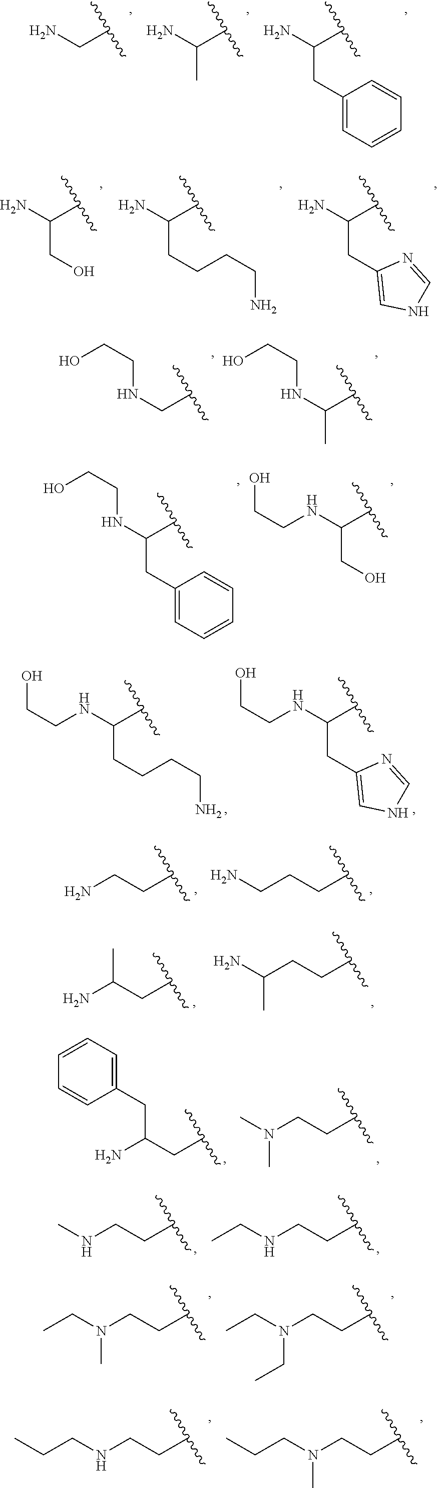

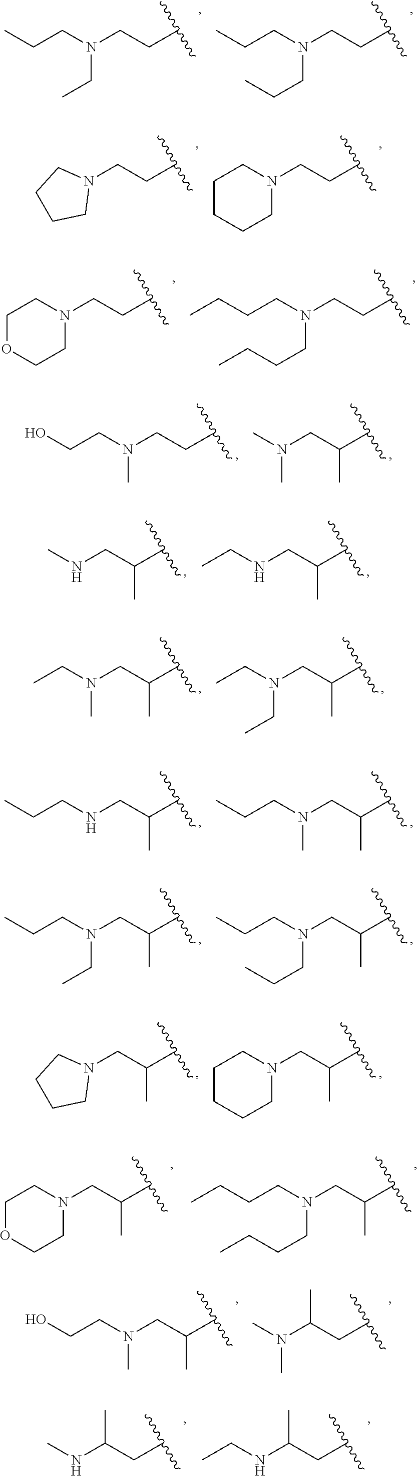

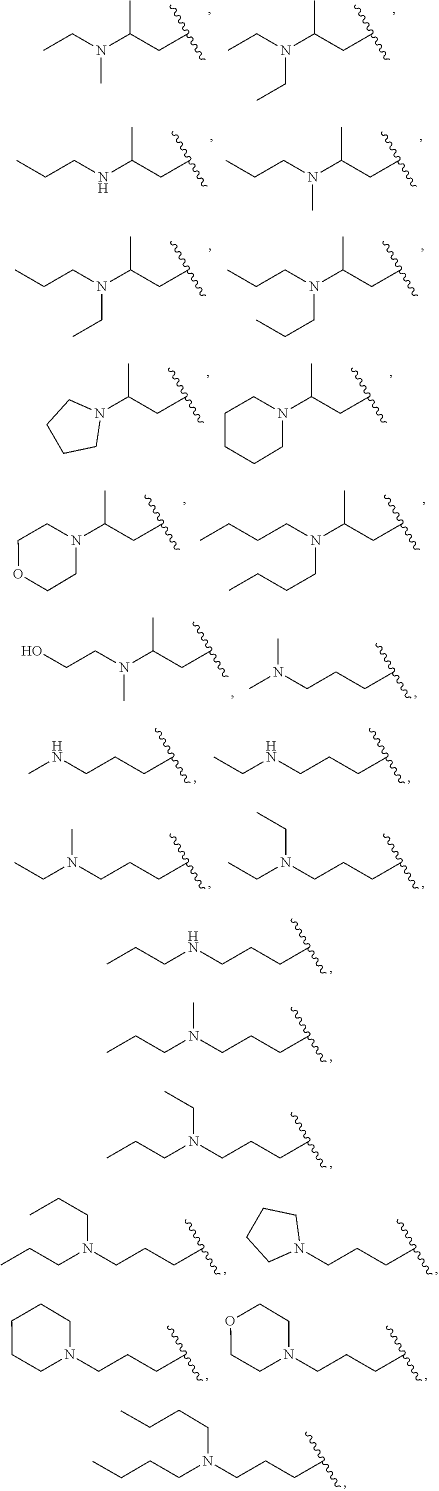

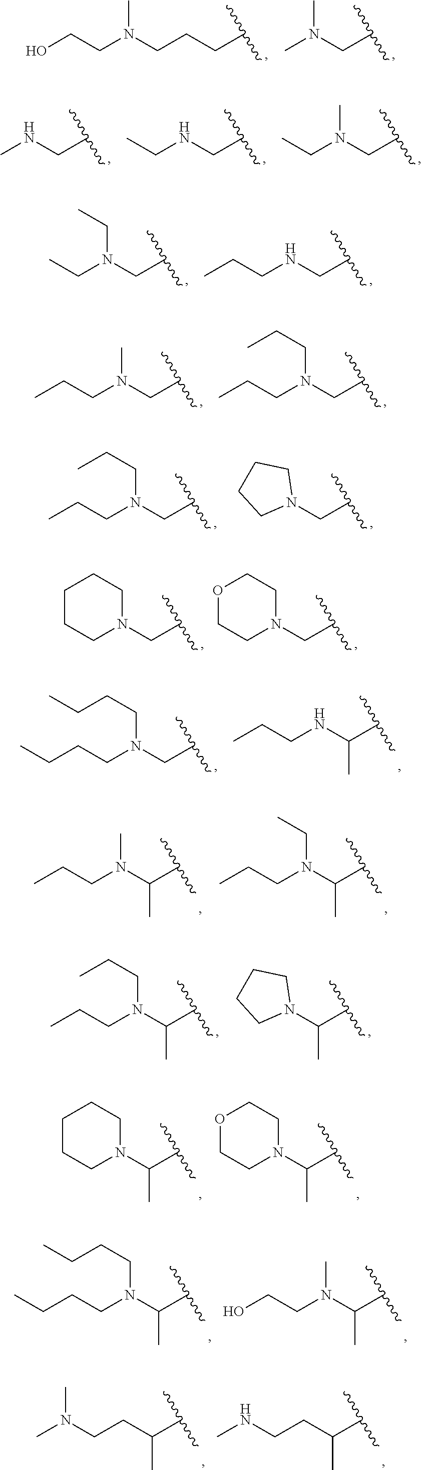

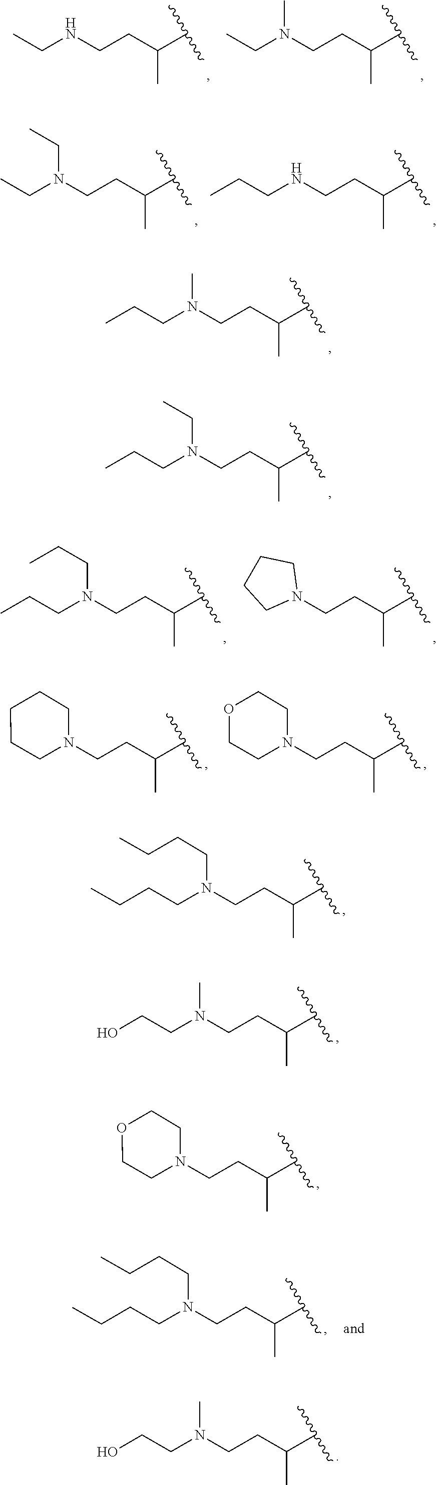

In certain preferred embodiments, Q is selected from

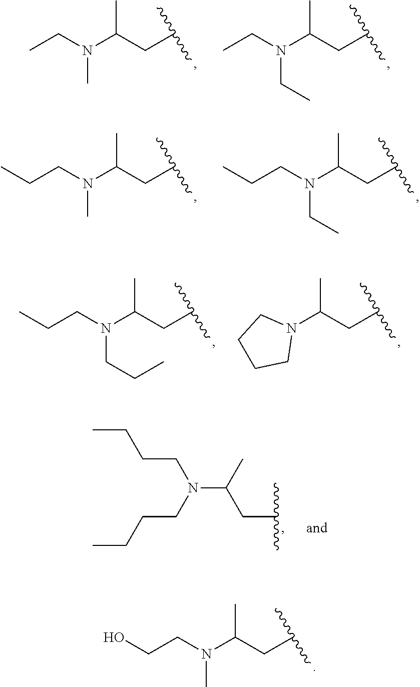

In other preferred embodiments, Q is

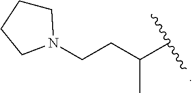 [SMILES: *C(C)CCN1CCCC1]

In any of the preceding embodiments, the salt of the lipidoid may be a pharmaceutically acceptable salt.

### Nanoparticle Compositions/Lipid Nanoformulations/Lipid-Based Carriers

In certain embodiments, provided herein is a nanoparticle composition comprising a lipidoid of the invention, or a pharmaceutically acceptable salt thereof. In certain embodiments, the nanoparticle composition comprises a plurality of lipidoids of the invention, or a pharmaceutically acceptable salt thereof.

As used here, “nanoparticle composition” is used interchangeably with the terms “lipid-based carrier,” “lipid nanoformulation,” and “lipid nanoparticle.”

In certain embodiments, the nanoparticle composition further comprises a lipid. In certain such embodiments, the lipid is a cationic, anionic, ionizable, or zwitterionic lipid.

In some embodiments, compounds described herein are formulated into a lipid-based carrier (or lipid nanoformulation). In some embodiments, the lipid-based carrier (or lipid nanoformulation) is a liposome or a lipid nanoparticle (LNP). In one embodiment, the lipid-based carrier is an LNP.

In some embodiments, the lipid-based carrier (or lipid nanoformulation) comprises a cationic lipid (e.g., an ionizable lipid), a non-cationic lipid (e.g., phospholipid), a structural lipid (e.g., cholesterol), and a PEG-modified lipid. In some embodiments, the lipid-based carrier (or lipid nanoformulation) contains one or more compounds described herein, or a pharmaceutically acceptable salt thereof.

All above descriptions and all embodiments discussed in the above aspects relating to the aspects of the lipid compounds, including the compounds covered by formula (I), and the exemplary formulas for lipids having ionizable head groups are all applicable to these aspects of the invention relating to the lipid-based carriers (or a lipid nanoformulation).

As described herein, suitable compounds to be used in the lipid-based carrier (or lipid nanoformulation) include all the isomers and isotopes of the compounds described above, as well as all the pharmaceutically acceptable salts, solvates, or hydrates thereof, and all crystal forms, crystal form mixtures, and anhydrides or hydrates.

In addition to one or more compounds described herein, the lipid-based carrier (or lipid nanoformulation) may further include a second lipid. In some embodiments, the second lipid is a cationic lipid, a non-cationic (e.g., neutral, anionic, or zwitterionic) lipid, or an ionizable lipid.

One or more naturally occurring and/or synthetic lipid compounds may be used in the preparation of the lipid-based carrier (or lipid nanoformulation).

The lipid-based carrier (or lipid nanoformulation) may contain positively charged (cationic) lipids, neutral lipids, negatively charged (anionic) lipids, or a combination thereof.

In some embodiments, the lipid nanoparticle of the invention may be conjugated to a targeting moiety (e.g., an antibody or antigen-binding fragment thereof) through a linking group. Various linking groups known in the art may be used in the lipid nanoparticles of the invention, and can comprise one or more of optionally substituted alkylene, optionally substituted heteroalkylene, optionally substituted alkenylene, optionally substituted heteroalkenylene, optionally substituted alkynylene, optionally substituted heteroalkynylene, optionally substituted cycloalkylene, optionally substituted heterocycloalkylene, optionally substituted arylene, optionally substituted heteroarylene, a peptide moiety, a dipeptide moiety, —(C═O)—, a disulfide, a hydrazone, thioester, sulfone, sulfoxide, thiosulfinate, thiosulfonate, sulfate, sulfonate, sulfonylurea, ether, thioether, ester, amide, carbonate, carbamate, urea, sulfamide, succinimide, maleimide, phosphate, diphosphate, triazole, or a saccharide, or a combination thereof. Suitable linking groups are described, e.g., in WO 2024/015229, WO 2024/006272, and WO 2023/225359.

### Cationic Lipids (Positively Charged) and Ionizable Lipids

In some embodiments, the lipid-based carrier (or lipid nanoformulation) comprises one or more cationic lipids, e.g., a cationic lipid that can exist in a positively charged or neutral form depending on pH, or an amine-containing lipid that can be readily protonated. In some embodiments, the cationic lipid is a lipid capable of being positively charged, e.g., under physiological conditions.

Exemplary cationic lipids include one or more amine group(s) which bear the positive charge. Examples of positively charged (cationic) lipids include, but are not limited to, N,N′-dimethyl-N,N′-dioctacyl ammonium bromide (DDAB) and chloride DDAC), N-(1-(2,3-dioleyloxy)propyl)-N,N,N-trimethylammonium chloride (DOTMA), 3β-[N—(N′,N′-dimethylaminoethyl)carbamoyl) cholesterol (DC-chol), 1,2-dioleoyloxy-3-[trimethylammonio]-propane (DOTAP), 1,2-dioctadecyloxy-3-[trimethylammonio]-propane (DSTAP), and 1,2-dioleoyloxypropyl-3-dimethyl-hydroxy ethyl ammonium chloride (DORI), N,N-dioleyl-N,N-dimethylammonium chloride (DODAC), N,N-dimethyl-2,3-dioleyloxy)propylamine (DODMA), 1,2-Dioleoyl-3-Dimethylammonium-propane (DODAP), 1,2-Dioleoylcarbamyl-3-Dimethylammonium-propane (DOCDAP), 1,2-Dilineoyl-3-Dimethylammonium-propane (DLINDAP), 3-Dimethylamino-2-(Cholest-5-en-3-beta-oxybutan-4-oxy)-1-(cis,cis-9,12-octadecadienoxy)propane (CLinDMA), 2-[5′-(cholest-5-en-3-beta-oxy)-3′-oxapentoxy)-3-dimethyl-1-(cis, cis-9′,12′-octadecadienoxy)propane (CpLin DMA), N,N-Dimethyl-3,4-dioleyloxybenzylamine (DMOBA), and the cationic lipids described in e.g. Martin et al., Current Pharmaceutical Design, pages 1-394, which is herein incorporated by reference in its entirety. In some embodiments, the lipid-based carrier (or lipid nanoformulation) comprises more than one cationic lipid.

In some embodiments, the lipid-based carrier (or lipid nanoformulation) comprises a cationic lipid having an effective pKa over 6.0. In some embodiments, the lipid-based carrier (or lipid nanoformulation) further comprises a second cationic lipid having a different effective pKa (e.g., greater than the first effective pKa) than the first cationic lipid.

In some embodiments, cationic lipids that can be used in the lipid-based carrier (or lipid nanoformulation) include, for example those described in Table 4 of WO 2019/217941, which is incorporated by reference.

In some embodiments, the cationic lipid is an ionizable lipid (e.g., a lipid that is protonated at low pH, but that remains neutral at physiological pH). In some embodiments, the lipid-based carrier (or lipid nanoformulation) may comprise one or more additional ionizable lipids, different than the ionizable lipids described herein. Exemplary ionizable lipids include, but are not limited to,

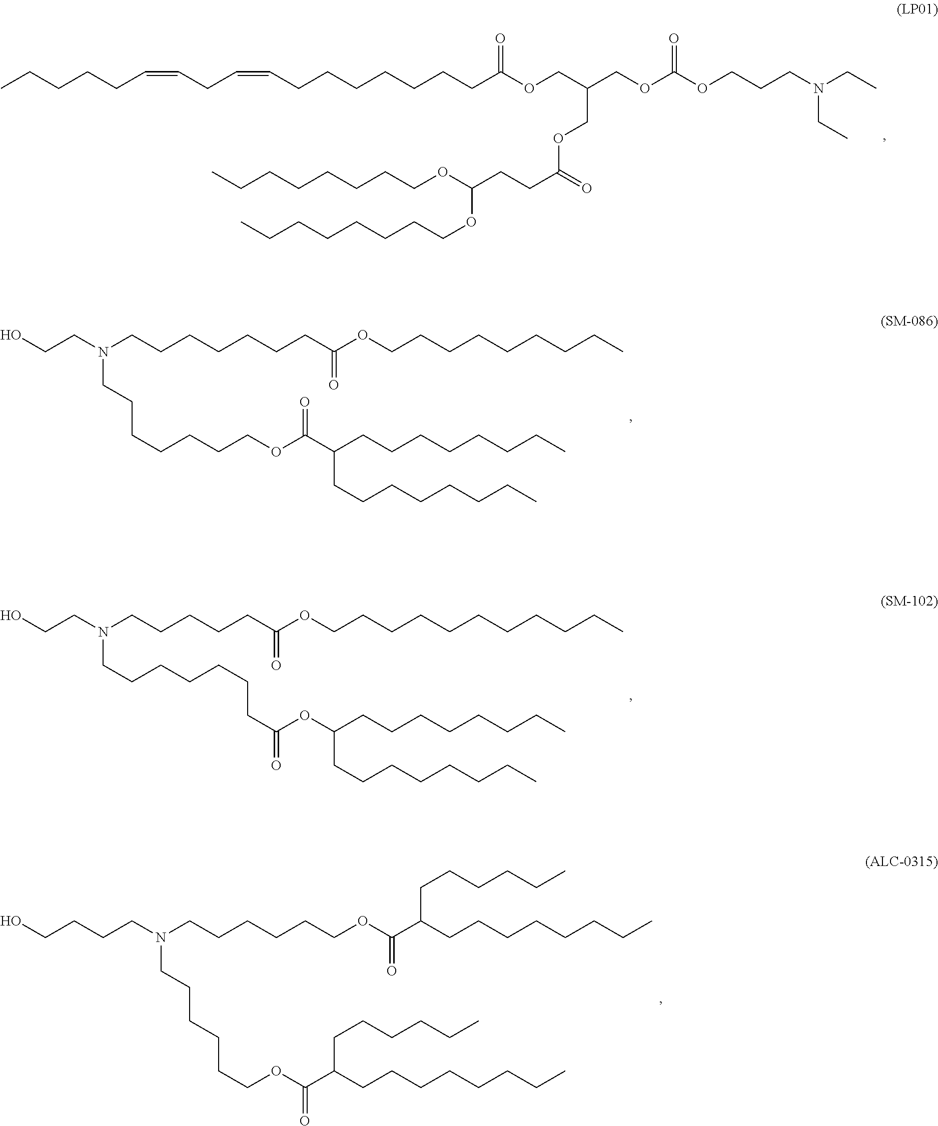 [SMILES: CCCCC/C=C\C/C=C\CCCCCCCC(=O)OCC(COC(=O)CCC(OCCCCCCCC)OCCCCCCCC)COC(=O)OCCCN(CC)CC ; CCCCCCCCCOC(=O)CCCCCCCN(CCO)CCCCCCCOC(=O)C(CCCCCCCC)CCCCCCCC ; CCCCCCCCCCCOC(=O)CCCCCN(CCO)CCCCCCCC(=O)OC(CCCCCCCC)CCCCCCCC ; CCCCCCCCC(CCCCCC)C(=O)OCCCCCCN(CCCCO)CCCCCCOC(=O)C(CCCCCC)CCCCCCCC]

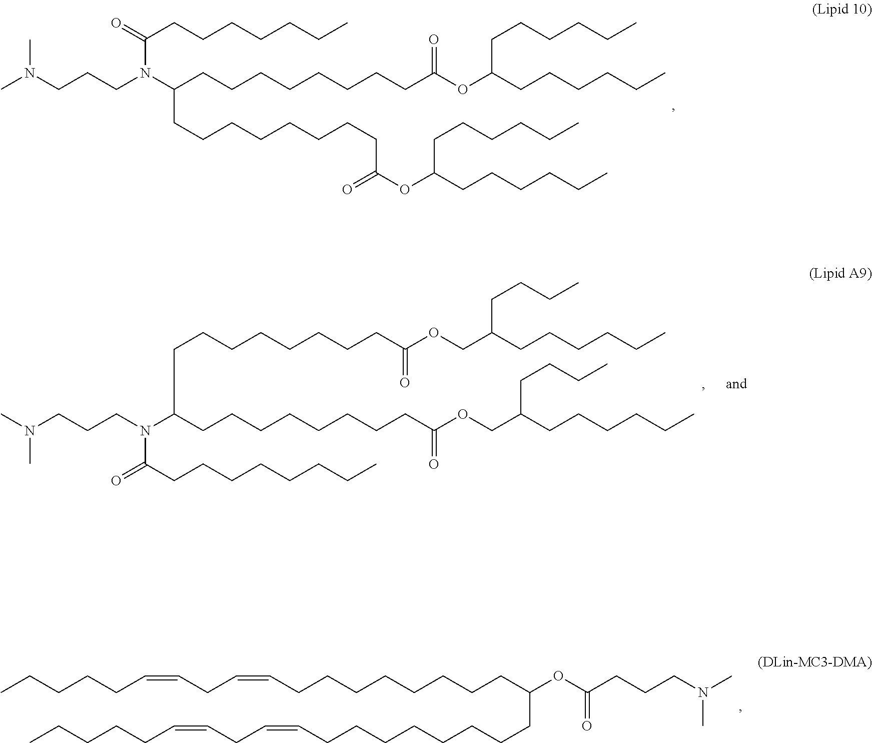

(see WO 2017/004143A1, which is incorporated herein by reference in its entirety).

In some embodiments, the lipid-based carrier (or lipid nanoformulation) further comprises one or more compounds described by WO 2021/113777 (e.g., a lipid of Formula (3) such as a lipid of Table 3 of WO 2021/113777), which is incorporated herein by reference in its entirety.

In one embodiment, the ionizable lipid is a lipid disclosed in Hou, X., et al. Nat Rev Mater 6, 1078-1094 (2021). https://doi.org/10.1038/s41578-021-00358-0 (e.g., L319, C12-200, and DLin-MC3-DMA), (which is incorporated by reference herein in its entirety).

Examples of other ionizable lipids that can be used in lipid-based carrier (or lipid nanoformulation) include, without limitation, one or more of the following formulas: X of US 2016/0311759; I of US 20150376115 or in US 2016/0376224; Compound 5 or Compound 6 in US 2016/0376224; I, IA, or II of U.S. Pat. No. 9,867,888; I, II or III of US 2016/0151284; I, IA, II, or IIA of US 2017/0210967; I-c of US 2015/0140070; A of US 2013/0178541; I of US 2013/0303587 or US 2013/0123338; I of US 2015/0141678; II, III, IV, or V of US 2015/0239926; I of US 2017/0119904; I or II of WO 2017/117528; A of US 2012/0149894; A of US 2015/0057373; A of WO 2013/116126; A of US 2013/0090372; A of US 2013/0274523; A of US 2013/0274504; A of US 2013/0053572; A of WO 2013/016058; A of WO 2012/162210; I of US 2008/042973; I, II, III, or IV of US 2012/01287670; I or II of US 2014/0200257; I, II, or III of US 2015/0203446; I or III of US 2015/0005363; I, IA, IB, IC, ID, II, IIA, IIB, IIC, IID, or III-XXIV of US 2014/0308304; of US 2013/0338210; I, II, III, or IV of WO 2009/132131; A of US 2012/01011478; I or XXXV of US 2012/0027796; XIV or XVII of US 2012/0058144; of US 2013/0323269; I of US 2011/0117125; I, II, or III of US 2011/0256175; I, II, III, IV, V, VI, VII, VIII, IX, X, XI, XII of US 2012/0202871; I, II, III, IV, V, VI, VII, VIII, X, XII, XIII, XIV, XV, or XVI of US 2011/0076335; I or II of US 2006/008378; I of WO2015/074085 (e.g., ATX-002); I of US 2013/0123338; I or X-A-Y-Z of US 2015/0064242; XVI, XVII, or XVIII of US 2013/0022649; I, II, or III of US 2013/0116307; I, II, or III of US 2013/0116307; I or II of US 2010/0062967; I-X of US 2013/0189351; I of US 2014/0039032; V of US 2018/0028664; I of US 2016/0317458; I of US 2013/0195920; 5, 6, or 10 of U.S. Pat. No. 10,221,127; III-3 of WO 2018/081480; I-5 or I-8 of WO 2020/081938; I of WO 2015/199952 (e.g., compound 6 or 22) and Table 1 therein; 18 or 25 of U.S. Pat. No. 9,867,888; A of US 2019/0136231; II of WO 2020/219876; 1 of US 2012/0027803; OF-02 of US 2019/0240349; 23 of U.S. Pat. No. 10,086,013; cKK-E12/A6 of Miao et al (2020); C12-200 of WO 2010/053572; 7C1 of Dahlman et al (2017); 304-013 or 503-013 of Whitehead et al; TS-P4C2 of U.S. Pat. No. 9,708,628; I of WO 2020/106946; I of WO 2020/106946; (1), (2), (3), or (4) of WO 2021/113777; and any one of Tables 1-16 of WO 2021/113777, all of which are incorporated herein by reference in their entirety.

In some embodiments, the lipid-based carrier (or lipid nanoformulation) further includes biodegradable ionizable lipids, for instance, (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate). See, e.g., lipids of WO 2019/067992, WO 2017/173054, WO 2015/095340, and WO 2014/136086, which are incorporated herein by reference in their entirety.

### Non-Cationic Lipids (e.g., Phospholipids)

In some embodiments, the lipid-based carrier (or lipid nanoformulation) further comprises one or more non-cationic lipids. In some embodiments, the non-cationic lipid is a phospholipid. In some embodiments, the non-cationic lipid is a phospholipid substitute or replacement. In some embodiments, the non-cationic lipid is a negatively charged (anionic) lipid.

Exemplary non-cationic lipids include, but are not limited to, distearoyl-sn-glycero-phosphoethanolamine, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoylphosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), monomethyl-phosphatidylethanolamine (such as 16-O-monomethyl PE), dimethyl-phosphatidylethanolamine (such as 16-O-dimethyl PE), 18-1-trans PE, 1-stearoyl-2-oleoyl-phosphatidyethanolamine (SOPE), hydrogenated soy phosphatidylcholine (HSPC), egg phosphatidylcholine (EPC), dioleoylphosphatidylserine (DOPS), sphingomyelin (SM), dimyristoyl phosphatidylcholine (DMPC), dimyristoyl phosphatidylglycerol (DMPG), distearoylphosphatidylglycerol (DSPG), dierucoylphosphatidylcholine (DEPC), palmitoyloleyolphosphatidylglycerol (POPG), dielaidoyl-phosphatidylethanolamine (DEPE), 1,2-dilauroyl-sn-glycero-3-phosphocholine (DLPC), Sodium 1,2-ditetradecanoyl-sn-glycero-3-phosphate (DMPA), phosphatidylcholine (lecithin), phosphatidylethanolamine, lysolecithin, lysophosphatidylethanolamine, phosphatidylserine, phosphatidylinositol, sphingomyelin, egg sphingomyelin (ESM), phosphatidylethanolamine (cephalin), cardiolipin, phosphatidic acid, cerebrosides, dicetylphosphate, lysophosphatidylcholine, dilinoleoylphosphatidylcholine, or mixtures thereof. It is understood that other diacylphosphatidylcholine and diacylphosphatidylethanolamine phospholipids can also be used. The acyl groups in these lipids are preferably acyl groups derived from fatty acids having C₁₀-C₂₄ carbon chains, e.g., lauroyl, myristoyl, paimitoyl, stearoyl, or oleoyl.

Additional exemplary lipids, in certain embodiments, include, without limitation, those described in Kim et al. (2020) dx.doi.org/10.1021/acs.nanolett.0c01386, which is incorporated herein by reference. Such lipids include, in some embodiments, plant lipids found to improve liver transfection with mRNA (e.g., DGTS).

In some embodiments, the lipid-based carrier (or lipid nanoformulation) may comprise a combination of distearoylphosphatidylcholine/cholesterol, dipalmitoylphosphatidylcholine/cholesterol, dimyrystoylphosphatidylcholine/cholesterol, 1,2-Dioleoyl-sn-glycero-3-phosphocholine (DOPC)/cholesterol, or egg sphingomyelin/cholesterol.

Other examples of suitable non-cationic lipids include, without limitation, nonphosphorous lipids such as, e.g., stearylamine, dodecylamine, hexadecylamine, acetyl palmitate, glycerol ricinoleate, hexadecyl stearate, isopropyl myristate, amphoteric acrylic polymers, triethanolamine-lauryl sulfate, alkyl-aryl sulfate polyethyloxylated fatty acid amides, dioctadecyl dimethyl ammonium bromide, ceramide, sphingomyelin, and the like. Other non-cationic lipids are described in WO 2017/099823 or US 2018/0028664, which are incorporated herein by reference in their entirety.

In one embodiment, the lipid-based carrier (or lipid nanoformulation) further comprises one or more non-cationic lipid that is oleic acid or a compound of Formula I, II, or IV of US 2018/0028664, which is incorporated herein by reference in its entirety.

The non-cationic lipid content can be, for example, 0-30% (mol) of the total lipid components present. In some embodiments, the non-cationic lipid content is 5-20% (mol) or 10-15% (mol) of the total lipid components present.

In some embodiments, the lipid-based carrier (or lipid nanoformulation) further comprises a neutral lipid, and the molar ratio of an ionizable lipid to a neutral lipid ranges from about 2:1 to about 8:1 (e.g., about 2:1, 3:1, 4:1, 5:1, 6:1, 7:1, or 8:1).

In some embodiments, the lipid-based carrier (or lipid nanoformulation) does not include any phospholipids.

In some embodiments, the lipid-based carrier (or lipid nanoformulation) can further include one or more phospholipids, and optionally one or more additional molecules of similar molecular shape and dimensions having both a hydrophobic moiety and a hydrophilic moiety (e.g., cholesterol).

Exemplary anionic lipids include dimyrystoyl-, dipalmitoyl-, and distearoyl-phasphatidylglycerol; dimyrystoyl-, dipalmitoyl-, and dipalmitoyl-phosphatidic acid; dimyrystoyl-, dipalmitoyl-, and dipalmitoyl-phosphatidylethanolamine; and their unsaturated diacyl and mixed acyl chain counterparts as well as cardiolipin.

Exemplary neutral lipids include DLPC (1,2-dilauroyl-sn-glycero-3-phosphocholine), DMPC (1,2-dimyristoyl-sn-glycero-3-phosphocholine), DPPC (1,2-dipalmitoyl-sn-glycero-3-phosphocholine), DSPC (1,2-distearoyl-sn-glycero-3-phosphocholine), DOPC (1,2-dioleoyl-sn-glycero-3-phosphocholine), DMPA (Sodium 1,2-ditetradecanoyl-sn-glycero-3-phosphate), DPPE (1,2-Dipalmitoyl-sn-glycero-3-phosphoethanolamine), and DOPE (1,2-dioleoyl-sn-glycero-3-phosphoethanolamine).

Exemplary phospholipids include, but are not limited to, phosphatidylcholine (lecithin), lysolecithin, lysophosphatidylethanol-amine, phosphatidylserine, phosphatidylinositol, sphingomyelin, phosphatidylethanolamine (cephalin), cardiolipin, phosphatidic acid, cerebrosides, dicetylphosphate, phosphatidylcholine, and dipalmitoyl-phosphatidylglycerol.

### Structural Lipids

The lipid-based carrier (or lipid nanoformulation) described herein may further comprise one or more structural lipids. As used herein, the term “structural lipid” refers to sterols (e.g., cholesterol and derivatives thereof) and to lipids containing sterol moieties.

Incorporation of structural lipids in the lipid nanoparticle may help mitigate aggregation of other lipid in the particle. Structural lipids can be selected from the group including but not limited to, cholesterol or cholesterol derivative, fecosterol, sitosterol, ergosterol, campesterol, stigmasterol, brassicasterol, tomatidine, tomatine, ursolic acid, alpha-tocopherol, hopanoids, phytosterols, steroids, and mixtures thereof. In some embodiments, the structural lipid is a sterol. In certain embodiments, the structural lipid is a steroid. In certain embodiments, the structural lipid is cholesterol. In certain embodiments, the structural lipid is an analog of cholesterol. In certain embodiments, the structural lipid is alpha-tocopherol.

In some embodiments, structural lipids may be incorporated into the lipid-based carrier at molar ratios ranging from about 0.1 to 1.0 (cholesterol phospholipid).

In some embodiments, sterols, when present, can include one or more of cholesterol or cholesterol derivatives, such as those described in WO 2009/127060 or US 2010/0130588, which are incorporated herein by reference in their entirety. Additional exemplary sterols include phytosterols, including those described in Eygeris et al. (2020), Nano Lett. 2020; 20(6):4543-4549, incorporated herein by reference.

In some embodiments, the structural lipid is a cholesterol derivative. Non-limiting examples of cholesterol derivatives include polar analogues such as 5a-cholestanol, 53-coprostanol, cholesteryl-(2′-hydroxy)-ethyl ether, cholesteryl-(4′-hydroxy)-butyl ether, and 6-ketocholestanol; non-polar analogues such as 5a-cholestane, cholestenone, 5a-cholestanone, 5p-cholestanone, and cholesteryl decanoate; and mixtures thereof. In some embodiments, the cholesterol derivative is a polar analogue, e.g., cholesteryl-(4′-hydroxy)-butyl ether. Exemplary cholesterol derivatives are described in WO 2009/127060 and US 2010/0130588, each of which is incorporated herein by reference in its entirety.

In some embodiments, the lipid-based carrier (or lipid nanoformulation) further comprises sterol in an amount of 0-50 mol % (e.g., 0-10 mol %, 10-20 mol %, 20-50 mol %, 20-30 mol %, 30-40 mol %, or 40-50 mol %) of the total lipid components.

### Polymers and Polyethylene Glycol (PEG) Lipids

In some embodiments, the lipid-based carrier (or lipid nanoformulation) may include one or more polymers or co-polymers, e.g., poly(lactic-co-glycolic acid) (PFAG) nanoparticles.

In some embodiments, the lipid-based carrier (or lipid nanoformulation) may include one or more polyethylene glycol (PEG) lipid (also referred to as a “PEGylated lipid”). Examples of useful PEG-lipids include, but are not limited to, 1,2-Diacyl-sn-Glycero-3-Phosphoethanolamine-N-[Methoxy(Polyethylene glycol)-350](mPEG 350 PE); 1,2-Diacyl-sn-Glycero-3-Phosphoethanolamine-N-[Methoxy(Polyethylene glycol)-550](mPEG 550 PE); 1,2-Diacyl-sn-Glycero-3-Phosphoethanolamine-N-[Methoxy(Polyethylene glycol)-750](mPEG 750 PE); 1,2-Diacyl-sn-Glycero-3-Phosphoethanolamine-N-[Methoxy(Polyethylene glycol)-1000](mPEG 1000 PE); 1,2-Diacyl-sn-Glycero-3-Phosphoethanolamine-N-[Methoxy(Polyethylene glycol)-2000](mPEG 2000 PE); 1,2-Diacyl-sn-Glycero-3-Phosphoethanolamine-N-[Methoxy(Polyethylene glycol)-3000](mPEG 3000 PE); 1,2-Diacyl-sn-Glycero-3-Phosphoethanolamine-N-[Methoxy(Polyethylene glycol)-5000](mPEG 5000 PE); N-Acyl-Sphingosine-1-[Succinyl(Methoxy Polyethylene Glycol) 750](mPEG 750 Ceramide); N-Acyl-Sphingosine-1-[Succinyl(Methoxy Polyethylene Glycol) 2000](mPEG 2000 Ceramide); and N-Acyl-Sphingosine-1-[Succinyl(Methoxy Polyethylene Glycol) 5000](mPEG 5000 Ceramide). In some embodiments, the PEG lipid is a polyethyleneglycol-diacylglycerol (i.e., polyethyleneglycol diacylglycerol (PEG-DAG), PEG-cholesterol, or PEG-DMB) conjugate.

In some embodiments, the lipid-based carrier (or nanoformulation) includes one or more conjugated lipids (such as PEG-conjugated lipids or lipids conjugated to polymers described in Table 5 of WO 2019/217941, which is incorporated herein by reference in its entirety). In some embodiments, the one or more conjugated lipids is formulated with one or more ionic lipids (e.g., non-cationic lipid such as a neutral or anionic, or zwitterionic lipid); and one or more sterols (e.g., cholesterol).

The PEG conjugate can comprise a PEG-dilaurylglycerol (C12), a PEG-dimyristylglycerol (C14), a PEG-dipalmitoylglycerol (C16), a PEG-disterylglycerol (C18), PEG-dilaurylglycamide (C12), PEG-dimyristylglycamide (C14), PEG-dipalmitoylglycamide (C16), and PEG-disterylglycamide (C18).

In some embodiments, conjugated lipids, when present, can include one or more of PEG-diacylglycerol (DAG) (such as 1-(monomethoxy-polyethyleneglycol)-2,3-dimyristoylglycerol (PEG-DMG)), PEG-dialkyloxypropyl (DAA), PEG-phospholipid, PEG-ceramide (Cer), a pegylated phosphatidylethanoloamine (PEG-PE), PEG succinate diacylglycerol (PEGS-DAG) (such as 4-0-(2′,3′-di(tetradecanoyloxy)propyl-1-0-(w-methoxy(polyethoxy)ethyl) butanedioate (PEG-S-DMG)), PEG dialkoxypropylcarbam, N-(carbonyl-methoxypolyethylene glycol 2000)-1,2-distearoyl-sn-glycero-3-phosphoethanolamine sodium salt, and those described in Table 2 of WO 2019/051289 (which is herein incorporated by reference in its entirety), and combinations of the foregoing.

Additional exemplary PEG-lipid conjugates are described, for example, in U.S. Pat. Nos. 5,885,613, 6,287,591, US 2003/0077829, US 2003/0077829, US 2005/0175682, US 2008/0020058, US 2011/0117125, US 2010/0130588, US 2016/0376224, US 2017/0119904, US 2018/0028664, and WO 2017/099823, all of which are incorporated herein by reference in their entirety.

In some embodiments, the PEG-lipid is a compound of Formula III, III-a-I, III-a-2, III-b-1, III-b-2, or V of US 2018/0028664, which is incorporated herein by reference in its entirety. In some embodiments, the PEG-lipid is of Formula II of US 2015/0376115 or US 2016/0376224, both of which are incorporated herein by reference in their entirety. In some embodiments, the PEG-DAA conjugate can be, for example, PEG-dilauryloxypropyl, PEG-dimyristyloxypropyl, PEG-dipalmityloxypropyl, or PEG-distearyloxypropyl. In some embodiments, the PEG-lipid includes one of the following:

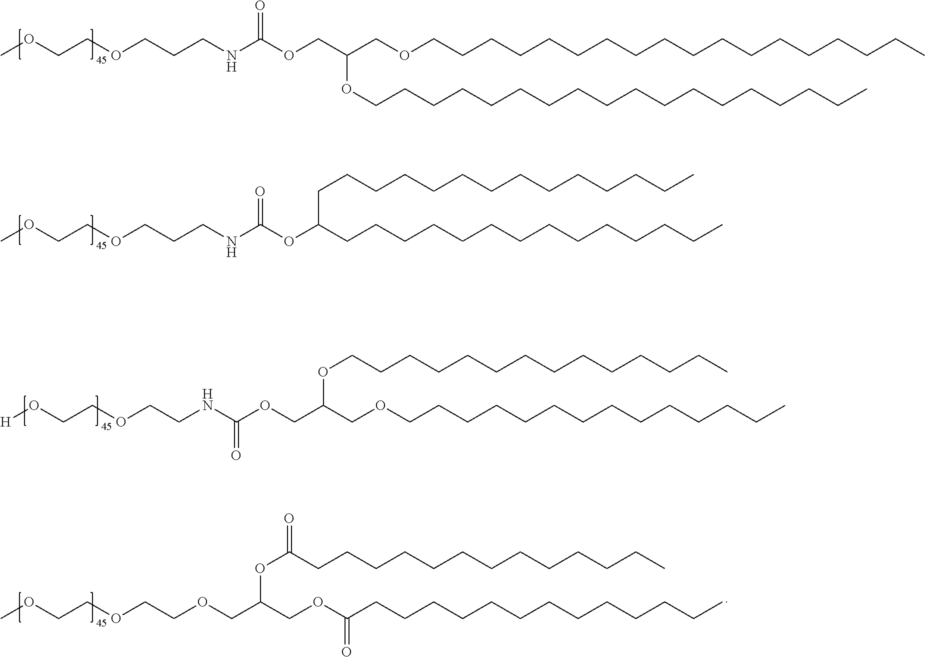

In some embodiments, lipids conjugated with a molecule other than a PEG can also be used in place of PEG-lipid. For example, polyoxazoline (POZ)-lipid conjugates, polyamide-lipid conjugates (such as ATTA-lipid conjugates), and cationic-polymer lipid (GPL) conjugates can be used in place of or in addition to the PEG-lipid.

Exemplary conjugated lipids, e.g., PEG-lipids, (POZ)-lipid conjugates, ATTA-lipid conjugates and cationic polymer-lipids, include those described in Table 2 of WO 2019/051289A9, which is incorporated herein by reference in its entirety.

In some embodiments, the conjugated lipid (e.g., the PEGylated lipid) can be present in an amount of 0-20 mol % of the total lipid components present in the lipid-based carrier (or lipid nanoformulation). In some embodiments, the conjugated lipid (e.g., the PEGylated lipid) content is 0.5-10 mol % or 2-5 mol % of the total lipid components.

When needed, the lipid-based carrier (or lipid nanoformulation) described herein may be coated with a polymer layer to enhance stability in vivo (e.g., sterically stabilized LNPs).

Examples of suitable polymers include, but are not limited to, poly(ethylene glycol), which may form a hydrophilic surface layer that improves the circulation half-life of liposomes and enhances the amount of lipid nanoformulations (e.g., liposomes or LNPs) that reach therapeutic targets. See, e.g., Working et al. J Pharmacol Exp Ther, 289: 1128-1133 (1999); Gabizon et al., J Controlled Release 53: 275-279 (1998); Adlakha Hutcheon et al., Nat Biotechnol 17: 775-779 (1999); and Koning et al., Biochim Biophys Acta 1420: 153-167 (1999), which are incorporated herein by reference in their entirety.

In certain embodiments, the nanoparticle composition further comprises a PEGylated lipid, a sterol, a phospholipid, and/or a neutral lipid.

### Percentages of Lipid Nanoformulation Components

In some embodiments, the lipid-based carrier (or lipid nanoformulation) comprises one or more of the compounds described herein, optionally a non-cationic lipid (e.g., a phospholipid), a sterol, a neutral lipid, and/or optionally conjugated lipid (e.g., a PEGylated lipid) that inhibits aggregation of particles. The relative amounts of these components can be varied independently and to achieve desired properties. For example, in some embodiments, the ionizable lipid including the lipid compounds described herein is present in an amount from about 20 mol % to about 100 mol % (e.g., 20-90 mol %, 20-80 mol %, 20-70 mol %, 25-100 mol %, 30-70 mol %, 30-60 mol %, 30-40 mol %, 40-50 mol %, or 50-90 mol %) of the total lipid and lipidoid components; a non-cationic lipid (e.g., phospholipid) is present in an amount from about 0 mol % to about 50 mol % (e.g., 0-40 mol %, 0-30 mol %, 5-50 mol %, 5-40 mol %, 5-30 mol %, or 5-10 mol %) of the total lipid and lipidoid components, a conjugated lipid (e.g., a PEGylated lipid) in an amount from about 0.5 mol % to about 20 mol % (e.g., 1-10 mol % or 5-10%) of the total lipid and lipidoid components, and a sterol in an amount from about 0 mol % to about 60 mol % (e.g., 0-50 mol %, 10-60 mol %, 10-50 mol %, 15-60 mol %, 15-50 mol %, 20-50 mol %, 20-40 mol %) of the total lipid and lipidoid components, provided that the total mol % of the lipid component does not exceed 100%.

In some embodiments, the lipid-based carrier (or lipid nanoformulation) comprises about 25-100 mol % of the ionizable lipid including the lipid and lipidoid compounds described herein, about 0-50 mol % phospholipid, about 0-50 mol % sterol, and about 0-10 mol % PEGylated lipid.

In one embodiment, the lipid-based carrier (or lipid nanoformulation) comprises about 25-100 mol % of the ionizable lipid including the lipid and lipidoid compounds described herein; about 0-40 mol % phospholipid (e.g., DSPC), about 0-50 mol % sterol (e.g., cholesterol), and about 0-10 mol % PEGylated lipid.

In some embodiments, the lipid-based carrier (or lipid nanoformulation) comprises about 30-60 mol % (e.g., about 35-55 mol %, or about 40-50 mol %) of the ionizable lipid including the lipid and lipidoid compounds described herein, about 0-30 mol % (e.g., 5-25 mol %, or 10-20 mol %) phospholipid, about 15-50 mol % (e.g., 18.5-48.5 mol %, or 30-40 mol %) sterol, and about 0-10 mol % (e.g., 1-5 mol %, or 1.5-2.5 mol %) PEGylated lipid.

In some embodiments, molar ratios of ionizable lipid/sterol/phospholipid (or another structural lipid)/PEG-lipid/additional components is varied in the following ranges: ionizable lipid (25-100%); phospholipid (DSPC) (0-40%); sterol (0-50%); and PEG lipid (0-5%).

In some embodiments, the lipid-based carrier (or lipid nanoformulation) comprises, by mol % or wt % of the total lipid and lipidoid components, 50-75% ionizable lipid (including the lipid and lipidoid compounds as described herein), 20-40% sterol (e.g., cholesterol or derivative), 0 to 10% non-cationic-lipid, and 1-10% conjugated lipid (e.g., the PEGylated lipid).

In some embodiments, the lipidoid compound described herein is a component of the lipid-based carrier (or lipid nanoformulation, or nanoparticle composition) and comprises from 10 mol % to 95 mol %, from 10 mol % to 90 mol %, from 10 mol % to 80 mol %, from 10 mol % to 70 mol %, from 10 mol % to 60 mol %, from 20 mol % to 55 mol %, from 20 mol % to 45 mol %, 20 mol % to 40 mol %, from 25 mol % to 50 mol %, from 25 mol % to 45 mol %, from 30 mol % to 50 mol %, from 30 mol % to 45 mol %, from 30 mol % to 40 mol %, from 35 mol % to 45 mol %, or from 37 mol % to 42 mol % (or any fraction of these ranges) of the total lipid and lipidoid components.

In some embodiments, where the lipid-based carrier (or lipid nanoformulation) contains a mixture of phospholipid and sterol (e.g. cholesterol or derivative), the mixture may be present up to 40 mol %, 45 mol %, 50 mol %, 55 mol %, or 60 mol % of the total lipid and lipidoid components.

In some embodiments, the phospholipid component in the mixture may be present from 2 mol % to 20 mol %, from 2 mol % to 15 mol %, from 2 mol % to 12 mol %, from 4 mol % to 15 mol %, from 4 mol % to 10 mol %, from 5 mol % to 10 mol %, (or any fraction of these ranges) of the total lipid and lipidoid components. In some embodiments, the lipid-based carrier (or lipid nanoformulation or nanoparticle composition) is substantially free of a phospholipid. In certain embodiments, the lipid-based carrier (or lipid nanoformulation or nanoparticle composition) is substantially free of distearolyphosphatidycholine (DSPC).

In some embodiments, the sterol component (e.g. cholesterol or derivative) in the mixture may comprise from 25 mol % to 45 mol %, from 25 mol % to 40 mol %, from 25 mol % to 35 mol %, from 25 mol % to 30 mol %, from 30 mol % to 45 mol %, from 30 mol % to 40 mol %, from 30 mol % to 35 mol %, from 35 mol % to 40 mol %, from 27 mol % to 37 mol %, or from 27 mol % to 35 mol % (or any fraction of these ranges) of the total lipid and lipidoid components.

In some embodiments, where the lipid-based carrier (or lipid nanoformulation) is phospholipid-free, the sterol component (e.g. cholesterol or derivative) may be present up to 25 mol %, 30 mol %, 35 mol %, 40 mol %, 45 mol %, 50 mol %, 55 mol %, or 60 mol % of the total lipid and lipidoid components. For instance, the sterol component (e.g. cholesterol or derivative) may be present from 25 mol % to 65 mol %, from 25 mol % to 60 mol %, from 25 mol % to 55 mol %, from 25 mol % to 50 mol %, from 25 mol % to 45 mol %, from 25 mol % to 40 mol %, from 30 mol % to 45 mol %, from 30 mol % to 40 mol %, from 35 mol % to 45 mol %, from 30 mol % to 35 mol %, or from 35 mol % to 40 mol % (or any fraction thereof or range therein) of the total lipid and lipidoid components.

In some embodiments, the non-ionizable lipid components in the lipid-based carrier (or lipid nanoformulation) may be present from 5 mol % to 90 mol %, from 10 mol % to 85 mol %, or from 20 mol % to 80 mol % (or any fraction of these ranges) of the total lipid and lipidoid components.

The ratio of total lipid components to the cargo (e.g., an encapsulated therapeutic agent such as a nucleic acid) can be varied as desired. For example, the total lipid components to the cargo (mass or weight) ratio can be from about 10:1 to about 30:1. In some embodiments, the total lipid and lipidoid components to the cargo ratio (mass/mass ratio; w/w ratio) can be in the range of from about 1:1 to about 25:1, from about 10:1 to about 14:1, from about 3:1 to about 15:1, from about 4:1 to about 10:1, from about 5:1 to about 9:1, or about 6:1 to about 9:1. The amounts of total lipid components and the cargo can be adjusted to provide a desired N/P ratio, for example, N/P ratio of 3, 4, 5, 6, 7, 8, 9, 10, 11, 12, 13, 14, 15, 16, 17, 18, 19, 20, 21, 22, 23, 24, 25, 26, 27, 28, 29, 30, or higher. Generally, the lipid-based carrier (or lipid nanoformulation)'s overall lipid content can range from about 5 mg/mL to about 30 mg/mL. Nitrogen:phosphate ratios (N:P ratio) is evaluated at values between 0.1 and 100.

In some embodiments, the lipid-based carrier (or lipid nanoformulation) includes the ionizable lipid compound as described herein, phospholipid, cholesterol, and a PEGylated lipid in a molar ratio of 50:10:38.5:1.5. In some embodiments, the lipid-based carrier (or lipid nanoformulation) includes the ionizable lipid compound as described herein, cholesterol and a PEGylated lipid in a molar ratio of 60:38.5:1.5.

In some embodiments of any of the aspects or embodiments herein, the lipid-based carrier (or lipid nanoformulation) further comprises a tissue targeting moiety. The tissue targeting moiety can be a peptide, oligosaccharide or the like, which can be used for the delivery of the lipid-based carrier (or lipid nanoformulation) to one or more specific tissues such as the liver. In some embodiments, the tissue targeting moiety is a ligand for liver specific receptors. In one embodiment, the ligand of liver specific receptors used for liver targeting is an oligosaccharide such as N-Acetylgalactosamine (GalNAc) which is covalently attached to a component of a lipid-based carrier (or lipid nanoformulation), e.g., PEG-lipid conjugates or the like. In some embodiments, the GalNAc is covalently attached to, for example, PEG-lipid conjugate. In some embodiments, the GalNAc is conjugated to DSPE-PEG2000. In some embodiments, the GalNAc-PEG-lipid conjugate is present in the lipid-based carrier (or lipid nanoformulation) at a molar percentage of 1.5%, 1.4%, 1.3%, 1.2%, 1.1%, 1.0%, 0.9%, 0.8%, 0.7%, 0.6%, 0.5%, 0.4%, 0.3%, 0.2%, or 0.1% of the total lipid. In some embodiments, the GalNAc-PEG-lipid conjugate is present in the lipid-based carrier (or lipid nanoformulation) at a molar percentage of 0.2% of the total lipid. In some embodiments, the GalNAc-PEG-lipid conjugate is present in the lipid-based carrier (or lipid nanoformulation) at a molar percentage of 0.3% of the total lipid. In some embodiments, the GalNAc-PEG-lipid conjugate is present in the lipid-based carrier (or lipid nanoformulation) at a molar percentage of 0.4% of the total lipid. In some embodiments, the GalNAc-PEG-lipid conjugate is present in the lipid-based carrier (or lipid nanoformulation) at a molar percentage of 0.5% of the total lipid. In some embodiments, the GalNAc-PEG-lipid conjugate is present in the lipid-based carrier (or lipid nanoformulation) at a molar percentage of 0.6% of the total lipid. In some embodiments, the GalNAc-PEG-lipid conjugate is present in the lipid-based carrier (or lipid nanoformulation) at a molar percentage of 0.7% of the total lipid. In some embodiments, the GalNAc-PEG-lipid conjugate is present in the lipid-based carrier (or lipid nanoformulation) at a molar percentage of 0.8% of the total lipid. In some embodiments, the GalNAc-PEG-lipid conjugate is present in the lipid-based carrier (or lipid nanoformulation) at a molar percentage of 0.9% of the total lipid. In some embodiments, the GalNAc-PEG-lipid conjugate is present in the lipid-based carrier (or lipid nanoformulation) at a molar percentage of 1.0% of the total lipid. In some embodiments, the GalNAc-PEG-lipid conjugate is present in the lipid-based carrier (or lipid nanoformulation) at a molar percentage of about 1.5% of the total lipid. In some embodiments, the GalNAc-PEG-lipid conjugate is present in the lipid-based carrier (or lipid nanoformulation) at a molar percentage of 2.0% of the total lipid.

### Properties of Lipid Nanoformulations

In some embodiments, the average particle diameter of the lipid-based carrier (or lipid nanoformulation) may be between 10 s of nm and 100 s of nm, e.g., measured by dynamic light scattering (DLS). In some embodiments, the average particle diameter of the lipid-based carrier (or lipid nanoformulation) ranges from about 1 mm to about 500 mm, from about 5 mm to about 200 mm, from about 10 mm to about 100 mm, from about 20 mm to about 80 mm, from about 25 mm to about 60 mm, from about 30 mm to about 55 mm, from about 35 mm to about 50 mm, from about 38 mm to about 42 mm, from about 40 nm to about 150 nm (such as about 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm), from about 50 nm to about 100 nm, from about 50 nm to about 90 nm, from about 50 nm to about 80 nm, from about 50 nm to about 70 nm, from about 50 nm to about 60 nm, from about 60 nm to about 100 nm, from about 60 nm to about 90 nm, from about 60 nm to about 80 nm, from about 60 nm to about 70 nm, from about 70 nm to about 100 nm, from about 70 nm to about 90 nm, from about 70 nm to about 80 nm, from about 80 nm to about 100 nm, from about 80 nm to about 90 nm, or from about 90 nm to about 100 nm.

The lipid-based carrier or lipid nanoformulation (e.g., liposome or LNP) may, in some instances, be relatively homogenous. A polydispersity index may be used to indicate the homogeneity of a lipid nanoformulation (e.g., liposome or LNP), e.g., the particle size distribution of the liposome or LNP. A small (e.g., less than 0.3) polydispersity index generally indicates a narrow particle size distribution. A lipid-based carrier or lipid nanoformulation (e.g., liposome or LNP) may have a polydispersity index from about 0 to about 0.25, such as 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, or 0.25. In some embodiments, the polydispersity index of the lipid-based carrier or lipid nanoformulation (e.g., liposome or LNP) may be from about 0.10 to about 0.20.

The zeta potential of a lipid-based carrier or a lipid nanoformulation (e.g., liposome or LNP) may be used to indicate the electrokinetic potential of the composition. In some embodiments, the zeta potential may describe the surface charge of a liposome or LNP. Lipid nanoformulations (e.g., liposomes or LNP) with relatively low charges, positive or negative, are generally desirable, as more highly charged species may interact undesirably with cells, tissues, and other elements in the body. In some embodiments, the zeta potential of a liposome or LNP may be from about −10 mV to about +20 mV, from about −10 mV to about +15 mV, from about −10 mV to about +10 mV, from about −10 mV to about +5 mV, from about −10 mV to about 0 mV, from about −10 mV to about −5 mV, from about −5 mV to about +20 mV, from about −5 mV to about +15 mV, from about −5 mV to about +10 mV, from about −5 mV to about +5 mV, from about −5 mV to about 0 mV, from about 0 mV to about +20 mV, from about 0 mV to about +15 mV, from about 0 mV to about +10 mV, from about 0 mV to about +5 mV, from about +5 mV to about +20 mV, from about +5 mV to about +15 mV, or from about +5 mV to about +10 mV.

The efficiency of encapsulation of a cargo such as a protein and/or nucleic acid, describes the amount of protein and/or nucleic acid that is encapsulated or otherwise associated with a lipid nanoformulation (e.g., liposome or LNP) after preparation, relative to the initial amount provided. The encapsulation efficiency is desirably high (e.g., at least 70%, 80%. 90%. 95%, close to 100%). The encapsulation efficiency may be measured, for example, by comparing the amount of protein or nucleic acid in a solution containing the liposome or LNP before and after breaking up the liposome or LNP with one or more organic solvents or detergents. An anion exchange resin may be used to measure the amount of free protein or nucleic acid (e.g., RNA) in a solution. Fluorescence may be used to measure the amount of free protein and/or nucleic acid (e.g., RNA) in a solution. For the liposome or LNP described herein, the encapsulation efficiency of a protein and/or nucleic acid may be at least 50%, for example 50%, 55%, 60%, 65%, 70%, 75%, 80%, 85%, 90%, 91%, 92%, 93%, 94%, 95%, 96%, 97%, 98%, 99%, or 100%. In some embodiments, the encapsulation efficiency may be at least 80%. In some embodiments, the encapsulation efficiency may be at least 90%. In some embodiments, the encapsulation efficiency may be at least 95%.

The lipid carrier or lipid nanoformulation may optionally include one or more coatings. In some embodiments, the lipid carrier or lipid nanoformulation (e.g., liposome or LNP) may be formulated in a capsule, film, or tablet having a coating. A capsule, film, or tablet including a composition described herein may have any useful size, tensile strength, hardness, or density.

Additional exemplary lipids, formulations, methods, and characterization of a lipid carrier or lipid nanoformulation (e.g., liposome or LNP) are taught by WO 2020/061457 and WO 2021/113777, which are incorporated herein by reference in their entirety. Further exemplary lipids, formulations, methods, and characterization of LNPs are taught by Hou et al. Lipid nanoparticles for mRNA delivery. Nat Rev Mater (2021). doi.org/10.1038/s41578-021-00358-0, which is incorporated herein by reference in its entirety (see, for example, exemplary lipids and lipid derivatives of FIG. 2 of Hou et al.).

In some embodiments, in vitro or ex vivo cell lipofections are performed using Lipofectamine MessengerMax (Thermo Fisher) or TransIT-mRNA Transfection Reagent (Mirus Bio). In certain embodiments, LNPs are formulated using the GenVoy_ILM ionizable lipid mix (Precision NanoSystems). In certain embodiments, LNPs are formulated using 2,2-dilinoleyl-4-dimethylaminoethyl-[1,3]-dioxolane (DLin-KC2-DMA) or dilinoleylmethyl-4-dimethylaminobutyrate (DLin-MC3-DMA or MC3), the formulation and in vivo use of which are taught in Jayaraman et al. Angew Chem Int Ed Engl 51(34):8529-8533 (2012), incorporated herein by reference in its entirety.

Lipid nanoformulations (e.g., liposome or LNP) optimized for the delivery of CRISPR-Cas systems, e.g., Cas9-gRNA RNP, gRNA, Cas9 mRNA, are described in WO 2019067992 and WO 2019067910, which are incorporated by reference in their entirety.

Additional specific lipid nanoformulations (e.g., liposome or LNP) useful for delivery of nucleic acid effector molecules are described in U.S. Pat. Nos. 8,158,601 and 8,168,775, which are incorporated by reference in their entirety.

A variety of methods can be used for preparing the lipid carrier or lipid nanoformulation (e.g., liposomes or LNPs) described herein. Such methods are known in the art or disclosed herein, for example, the methods described in Lichtenberg and Barenholz in Methods of Biochemical Analysis, 33:337-462 (1988), which is incorporated herein by reference in its entirety. See also Szoka et al., Ann. Rev. Biophys. Bioeng. 9:467 (1980); U.S. Pat. Nos. 4,235,871; 4,501,728; and 4,837,028; Liposomes, Marc J. Ostro, ed., Marcel Dekker, Inc., New York, 1983, Chapter 1; and Hope, et al., Chem. Phys. Lip. 40:89 (1986), which are incorporated herein by reference in their entirety. Small unilamellar vesicles (SUV, size <100 nm) can be prepared by a combination of standard methods of thin-film hydration and repeated extrusion.

Techniques for sizing the lipid carrier or lipid nanoformulations (e.g., liposomes or LNPs) to a desired size are well-known to one skilled in the art. See, e.g., U.S. Pat. No. 4,737,323, and Hope et al., Biochim. Biophys. Acta, 812: 55-65, which are incorporated by reference in their entirety. Sonicating a lipid nanoformulation (e.g., liposome or LNP) suspension either by bath or probe sonication produces a progressive size reduction down to small unilamellar vesicles less than about 50 nm in size. Homogenization or microfluidization are other methods which rely on shearing energy to fragment large lipid nanoformulations (e.g., liposomes or LNPs) into smaller ones. In a typical homogenization procedure, multilamellar vesicles are recirculated through a standard emulsion homogenizer until selected lipid nanoformulation (e.g., liposome or LNP) sizes, typically between about 100 and 500 nm, are observed. In both methods, the particle size distribution can be monitored by conventional laser-beam particle size discrimination.

Extrusion of lipid nanoformulations (e.g., liposomes or LNPs) through a small-pore polycarbonate membrane or an asymmetric ceramic membrane is a very effective method for reducing liposome or LNP sizes to a relatively well-defined size distribution. Typically, the suspension is cycled through the membrane one or more times until the desired liposome or LNP size distribution is achieved. The lipid-based carrier or lipid nanoformulations may be extruded through successively smaller-pore membranes, to achieve a gradual reduction in liposome or LNP size.

Any of the lipid-based carrier or lipid nanoformulations described herein can be analyzed by methods well-known to one skilled in the art to determine its physical and/or chemical features. For example, a phosphate assay can be used to determine the concentration of the lipid nanoformulations. One phosphate assay is based on the interaction between molybdate and malachite green dye. The main principle involves the reaction of inorganic phosphate with molybdate to form a colorless unreduced phosphomolybdate complex which is converted to a blue colored complex when reduced under acidic conditions. Phosphomolybdate gives 20 or 30 times more color when complexed with malachite green. The final product, reduced green soluble complex is measured by its absorbance at 620 nm and is a direct measure of inorganic phosphate in solution.

In some embodiments, the lipid-based carrier or lipid nanoformulations disclosed herein are tested for particle size, lipid concentration, and active agent encapsulation.

### Therapeutic Agents

In certain embodiments, the nanoparticle composition further comprises a therapeutic agent.

In certain such embodiments, the therapeutic agent is a nucleic acid molecule, such as a plasmid, an immunostimulatory oligonucleotide, an antisense oligonucleotide, an antagomir, an aptamer, a deoxyribozyme (DNAzyme), or a ribozyme. In certain embodiments, the nucleic acid molecule is DNA or RNA. In certain embodiments, the nucleic acid molecule is DNA; and the DNA is a linear DNA, circular DNA, single stranded DNA, or double stranded DNA. In alternative embodiments, wherein the nucleic acid molecule is RNA; the RNA is messenger RNA (mRNA), miRNA, siRNA or siRNA precursor, RNA aptamer, linear RNA, circular RNA, single stranded RNA, double stranded RNA, tRNA, microRNA (miRNA) or miRNA precursor, Dicer substrate small interfering RNA (dsiRNA), Dicer substrate RNA (dsRNA), short hairpin RNA (shRNA), asymmetric interfering RNA (aiRNA), guide RNA (gRNA), lncRNA, ncRNA, sncRNA, rRNA, snRNA, piRNA, snoRNA, snRNA, scaRNA, exRNA, scaRNA, Y RNA, or hnRNA, preferably mRNA. In yet further embodiments, the nucleic acid molecule comprises a phosphoramide, a phosphorothioate, a phosphorodithioate, an O-methylphosphoroamidate, a morpholino, a locked nucleic acid (LNA), a glycerol nucleic acid (GNA), a threose nucleic acid (TNA), or a peptide nucleic acid (PNA).

In some embodiments, the nucleic acid molecule (e.g., RNA or DNA) encodes a therapeutic peptide or polypeptide, operably linked to a promoter for a DNA. The therapeutic peptide or polypeptide may be, e.g., a transcription factor; a chromatin remodeling factor; an antigen; a hormone; an enzyme (such as a nuclease, e.g., an endonuclease, e.g., a nuclease element of a CRISPR system, e.g., a Cas9, dCas9, aCas9-nickase, CpfCas12a); a Crispr-linked enzyme, e.g., a base editor or prime editor; a mobile genetic element protein (e.g., a transposase, a retrotransposase, a recombinase, an integrase); a Gene Writer; a polymerase; a methylase; a demethylase; an acetylase; a deacetylase; a kinase; a phosphatase; a ligase; a deubiquitinase; an integrase; a recombinase; a topoisomerase; a gyrase; a helicase; a lysosomal acid hydrolase); an antibody; a receptor ligand; a receptor; a clotting factor; a membrane protein; a mitochondrial protein; a nuclear protein; an antibody or other protein scaffold binder such as a centyrin, darpin, or adnectin.

In some embodiments, the nucleic acid molecule (e.g., a DNA or RNA) encodes (if DNA) or is (if RNA) a non-coding RNA, e.g., one or more of a siRNA, a miRNA, long non-coding RNA, a piRNA, a snoRNA, a scaRNA, a tRNA, a rRNA, a therapeutic RNA aptamer, and a snRNA.

In some embodiments, the nucleic acid molecule can include a plurality of sequences. The plurality may be the same or different types. The plurality of sequences may be the same or different sequences of the same type.

All the nucleic acid molecules described herein can be chemically modified. The various modification strategy to the nucleic acid molecules are well known to one skilled in the art. In some embodiments, the nucleic acid molecule comprises one or more modifications such as pseudouridine, 5-bromouracil, 5-methylcytosine, peptide nucleic acid, xeno nucleic acid, morpholinos, locked nucleic acids, glycol nucleic acids, threose nucleic acids, dideoxynucleotides, cordycepin, 7-deaza-GTP, florophores (e.g. rhodamine or fluorescein linked to the sugar), thiol containing nucleotides, biotin linked nucleotides, fluorescent base analogs, CpG islands, methyl-7-guanosine, methylated nucleotides, inosine, thiouridine, pseudourdine, dihydrouridine, queuosine, or wyosine. In some embodiments, the antisense oligonucleotide may be a locked nucleic acid oligonucleotide (LNA). The term “locked nucleic acid (LNA)” refers to oligonucleotides that contain one or more nucleotide building blocks in which an extra methylene bridge fixes the ribose moiety either in the C3′-endo (beta-D-LNA) or C2′-endo (alpha-L-LNA) conformation (Grunweller A, Hartmann R K, BioDrugs, 21(4):235-243 (2007)).

Additional examples of the nucleic acid molecules (including tumor suppressor genes, antisense oligonucleotides, siRNA, miRNA, or shRNA) may be found in U.S. Published Patent Application No. 2007/0065499 and U.S. Pat. No. 7,780,882, which are incorporated by reference herein in their entireties.

In some embodiments, the nanoparticle composition can include a plurality of nucleic acid molecules, which may be the same or different types.

In some embodiments, the N:P ratio of the nucleic acid molecule-lipidoid composition ranges from 1:1 to 30:1, for instance from 3:1 to 20:1, from 3:1 to 15:1, from 3:1 to 10:1, or from 3:1 to 6:1. An N:P ratio refers to the molar ratio of the amines present in the plurality of lipidoids to the phosphates present in the nucleic acid molecule. In certain embodiments, the N:P ratio of the nucleic acid molecule—lipidoid composition ranges from 3:1 to 15:1.

In alternative embodiments, the therapeutic agent is a protein or small molecule drug.

In certain embodiments, the protein may be a peptide or polypeptide, e.g., a transcription factor; a chromatin remodeling factor; an antigen; a hormone; an enzyme (such as a nuclease, e.g., an endonuclease, e.g., a nuclease element of a CRISPR system, e.g., a Cas9, dCas9, aCas9-nickase, Cpf/Cas12a); a Crispr-linked enzyme, e.g., a base editor or prime editor; a mobile genetic element protein (e.g., a transposase, a retrotransposase, a recombinase, an integrase); a gene writer; a polymerase; a methylase; a demethylase; an acetylase; a deacetylase; a kinase; a phosphatase; a ligase; a deubiquitinase; an integrase; a recombinase; a topoisomerase; a gyrase; a helicase; a lysosomal acid hydrolase); an antibody; a receptor ligand; a receptor; a clotting factor; a membrane protein; a mitochondrial protein; a nuclear protein; an antibody or other protein scaffold binder, such as a centyrin, darpin, or adnectin.

In certain embodiments, the protein is a ribonucleoprotein (RNP) that a complex of ribonucleic acid and RNA-binding protein.

In certain embodiments, the protein is a recombinant cytokine such as IL-12.

In certain embodiments, the nanoparticle composition can include a plurality of protein molecules, which may be the same or different types.

In certain embodiments, the small molecule drug comprises an immune suppressive agent, an endosomal escape agent, or a combination of both.

In certain embodiments, the small molecule drug is an HDAC inhibitor, a kinase inhibitor, a cytotoxic molecule, a chromatin modulator, an RNAi modulator, transcription factor, an adjuvant, or a combination of two or more.

In some embodiments, the small molecule drug may be a compound with limited cell permeability properties. For instance, the small molecule drug may be a STING (simulator of interferon genes) modulator.

In some embodiments, the nanoparticle composition can include a plurality of small molecule drugs, which may be the same or different types.

In some embodiments, the therapeutic agent may be encapsulated in the LNP. For example, the therapeutic agent may be completely or partially located in the interior space of the LNPs, within the lipid layer/membrane, or associated with the exterior surface of the lipid layer/membrane. In some embodiments, incorporation of the therapeutic agent into the LNP protects the therapeutic agents from environments which may contain enzymes or chemicals or conditions that degrade the therapeutic agents and/or systems or receptors that cause the rapid excretion of the therapeutic agents. Moreover, incorporating the therapeutic agent into the LNP may promote uptake of the therapeutic agent, and hence, may enhance the therapeutic effect.

The ratio of total lipid and lipidoid components to the therapeutic agent can be varied as desired. For example, the total lipid and lipidoid components to the therapeutic agent (mass or weight) ratio can be from about 10:1 to about 30:1. In some embodiments, the total lipid and lipidoid components to the therapeutic agent ratio (mass/mass ratio; w/w ratio) can be in the range of from about 1:1 to about 25:1, from about 10:1 to about 14:1, from about 3:1 to about 15:1, from about 4:1 to about 10:1, from about 5:1 to about 9:1, or about 6:1 to about 9:1. The amounts of total lipid and lipidoid components and the therapeutic agent can be adjusted to provide a desired N:P ratio, for example, N:P ratio of 3, 4, 5, 6, 7, 8, 9, 10, 11, 12, 13, 14, 15, 16, 17, 18, 19, 20, 21, 22, 23, 24, 25, 26, 27, 28, 29, 30, or higher. Generally, the overall lipid and lipidoid content can range from about 5 mg/mL to about 30 mg/mL in the nanoparticle composition.

The nanoparticle composition may contain about 5 to about 95% by weight of the therapeutic agent. In some embodiments, the nanoparticle composition contains about 5%, about 10%, about 20, about 30, about 40, about 50, about 60, about 70, about 80, about 90, or about 95% by weight of the therapeutic agent. In some embodiments, the nanoparticle composition contains the therapeutic agent in an amount about 5-95%, about 5-90%, about 5-80%, about 5-70%, about 5-60%, about 5-50%, about 5-40%, about 5-30%, about 5-20%, about 5-10%, about 10-95%, about 10-90%, about 10-80%, about 10-70%, about 10-60%, about 10-50%, about 10-40%, about 10-30%, about 10-20%, about 20-95%, about 20-90%, about 20-80%, about 20-70%, about 20-60%, about 20-50%, about 20-40%, about 20-30%, about 30-95%, about 30-90%, about 30-80%, about 30-70%, about 30-60%, about 30-50%, about 30-40%, about 40-95%, about 40-90%, about 40-80%, about 40-70%, about 40-60%, about 40-50%, about 50-95%, about 50-90%, about 50-80%, about 50-70%, about 50-60%, about 60-95%, about 60-90%, about 60-80%, about 60-70%, about 70-95%, about 70-90%, about 70-80%, about 80-95%, about 80-90%, or about 90-95%, based on the weight of the nanoparticle composition.

In certain embodiments, the nanoparticle composition further comprises an antigen; wherein the antigen is a protein or a nucleic acid; the antigen is a protein; or the antigen is a nucleic acid.

In further embodiments, the nanoparticle composition further comprises an mRNA molecule comprising a nucleotide sequence that encodes an antigen.

The nanoparticle compositions described herein are useful for delivering a therapeutic agent. Accordingly, in certain embodiments, provided herein is a method of delivering a therapeutic agent, comprising administering to a subject in need thereof an effective amount of the nanoparticle composition of the invention that comprises a therapeutic agent.

The nanoparticle compositions described herein are useful for delivering an antigen. Accordingly, in certain embodiments, provided herein is a method of vaccination, comprising administering to a subject in need thereof an effective amount of a nanoparticle composition of the invention, wherein the nanoparticle composition comprises an antigen.

### Pharmaceutical Compositions

In certain aspects, the present invention provides a pharmaceutical composition comprising a nanoparticle composition of the invention, in combination with a pharmaceutically acceptable carrier.

As used throughout this section, a “lipidoid composition” can refer to a nanoparticle composition comprising a lipidoid compound, e.g., a lipidoid compound of the invention.

The compositions and methods of the present invention may be utilized to treat an individual in need thereof. The pharmaceutical composition described herein may comprise a therapeutic or prophylactic composition, or any combination thereof. In certain embodiments, the individual is a mammal such as a human, or a non-human mammal. When administered to an animal, such as a human, the nanoparticle composition or the lipidoid composition is preferably administered as a pharmaceutical composition comprising, for example, a lipidoid composition and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil, or injectable organic esters. In preferred embodiments, when such pharmaceutical compositions are for human administration, particularly for invasive routes of administration (i.e., routes, such as injection or implantation, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as tablet, capsule (including sprinkle capsule and gelatin capsule), granule, lyophile for reconstitution, powder, solution, syrup, suppository, injection or the like. The composition can also be present in a transdermal delivery system, e.g., a skin patch. The composition can also be present in a solution suitable for topical administration, such as a lotion, cream, or ointment.

A pharmaceutically acceptable carrier can contain physiologically acceptable agents that act, for example, to stabilize, increase solubility or to increase the absorption of a lipidoid composition. Such physiologically acceptable agents include, for example, carbohydrates, such as glucose, sucrose or dextrans, antioxidants, such as ascorbic acid or glutathione, chelating agents, low molecular weight proteins or other stabilizers or excipients.

The choice of a pharmaceutically acceptable carrier, including a physiologically acceptable agent, depends, for example, on the route of administration of the composition. The preparation or pharmaceutical composition can be a self-emulsifying drug delivery system or a self-microemulsifying drug delivery system. The pharmaceutical composition (preparation) also can be a liposome or other polymer matrix, which can have incorporated therein, for example, a lipidoid composition. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those lipidoid compositions, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A pharmaceutical composition (preparation) can be administered to a subject by any of a number of routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); subcutaneously; transdermally (for example as a patch applied to the skin); and topically (for example, as a cream, ointment or spray applied to the skin). The lipidoid composition may also be formulated for inhalation. In certain embodiments, a lipidoid composition may be simply dissolved or suspended in sterile water. Details of appropriate routes of administration and compositions suitable for same can be found in, for example, U.S. Pat. Nos. 6,110,973, 5,763,493, 5,731,000, 5,541,231, 5,427,798, 5,358,970 and 4,172,896, as well as in patents cited therein.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the lipidoid composition which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an active composition, such as a lipidoid (e.g., nanoparticle) composition as described herein, with the carrier and, optionally, one or more accessory ingredients. In general, the formulations are prepared by uniformly and intimately bringing into association a lipidoid (e.g., nanoparticle) composition as described herein with liquid carriers, or finely divided solid carriers, or both, and then, if necessary, shaping the product.

Formulations of the invention suitable for oral administration may be in the form of capsules (including sprinkle capsules and gelatin capsules), cachets, pills, tablets, lozenges (using a flavored basis, usually sucrose and acacia or tragacanth), lyophile, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a lipidoid (e.g., nanoparticle) composition as described herein of the present invention as an active ingredient. Lipidoid compositions may also be administered as a bolus, electuary or paste.

To prepare solid dosage forms for oral administration (capsules (including sprinkle capsules and gelatin capsules), tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium lipidoid compositions; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; (10) complexing agents, such as, modified and unmodified cyclodextrins; and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered lipidoid composition moistened with an inert liquid diluent.

The tablets, and other solid dosage forms of the pharmaceutical compositions, such as dragees, capsules (including sprinkle capsules and gelatin capsules), pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms useful for oral administration include pharmaceutically acceptable emulsions, lyophiles for reconstitution, microemulsions, solutions, suspensions, syrups and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active lipidoid compositions, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Dosage forms for the topical or transdermal administration include powders, sprays, ointments, pastes, creams, lotions, gels, solutions, patches and inhalants. The active lipidoid composition may be mixed under sterile conditions with a pharmaceutically acceptable carrier, and with any preservatives, buffers, or propellants that may be required.

The ointments, pastes, creams and gels may contain, in addition to an active lipidoid composition, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active lipidoid composition, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a lipidoid composition to the body. Such dosage forms can be made by dissolving or dispersing the active lipidoid composition in the proper medium. Absorption enhancers can also be used to increase the flux of the lipidoid composition across the skin. The rate of such flux can be controlled by either providing a rate controlling membrane or dispersing the lipidoid composition in a polymer matrix or gel.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion. Pharmaceutical compositions suitable for parenteral administration comprise one or more active lipidoid compositions in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsulated matrices of the lipidoid compositions in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

For use in the methods of this invention, active lipidoid compositions can be given per se or as a pharmaceutical composition containing, for example, 0.1 to 99.5% (more preferably, 0.5 to 90%) of active ingredient in combination with a pharmaceutically acceptable carrier.

Methods of introduction may also be provided by rechargeable or biodegradable devices. Various slow release polymeric devices have been developed and tested in vivo in recent years for the controlled delivery of drugs, including proteinaceous biopharmaceuticals. A variety of biocompatible polymers (including hydrogels), including both biodegradable and non-degradable polymers, can be used to form an implant for the sustained release of a lipidoid composition at a particular target site.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular lipidoid composition or combination of lipidoid compositions employed, or the ester, salt or amide thereof, the route of administration, the time of administration, the rate of excretion of the particular lipidoid composition(s) being employed, the duration of the treatment, other drugs, lipidoid compositions and/or materials used in combination with the particular lipidoid composition(s) employed, the age, sex, weight, condition, general health and prior medical history of the patient being treated, and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition or lipidoid composition at levels lower than that required in order to achieve the desired therapeutic effect and gradually increase the dosage until the desired effect is achieved. By “therapeutically effective amount” is meant the concentration of a lipidoid composition that is sufficient to elicit the desired therapeutic effect. It is generally understood that the effective amount of the lipidoid composition will vary according to the weight, sex, age, and medical history of the subject. Other factors which influence the effective amount may include, but are not limited to, the severity of the patient's condition, the disorder being treated, the stability of the lipidoid composition, and, if desired, another type of therapeutic agent being administered with the lipidoid composition of the invention. A larger total dose can be delivered by multiple administrations of the agent. Methods to determine efficacy and dosage are known to those skilled in the art (Isselbacher et al. (1996) Harrison's Principles of Internal Medicine 13 ed., 1814-1882, herein incorporated by reference).

In general, a suitable daily dose of an active lipidoid composition used in the compositions and methods of the invention will be that amount of the lipidoid composition that is the lowest dose effective to produce a therapeutic effect. Such an effective dose will generally depend upon the factors described above.

If desired, the effective daily dose of the active lipidoid composition may be administered as one, two, three, four, five, six or more sub-doses administered separately at appropriate intervals throughout the day, optionally, in unit dosage forms. In certain embodiments of the present invention, the active lipidoid composition may be administered two or three times daily. In preferred embodiments, the active lipidoid composition will be administered once daily.

The patient receiving this treatment is any animal in need, including primates, in particular humans; and other mammals such as equines, cattle, swine, sheep, cats, and dogs; poultry; and pets in general.

In certain embodiments, lipidoid compositions of the invention may be used alone or conjointly administered with another type of therapeutic agent.

The present disclosure includes the use of pharmaceutically acceptable salts of lipidoids of the invention in the compositions and methods of the present invention. In certain embodiments, contemplated salts of the invention include, but are not limited to, alkyl, dialkyl, trialkyl or tetra-alkyl ammonium salts. In certain embodiments, contemplated salts of the invention include, but are not limited to, L-arginine, benenthamine, benzathine, betaine, calcium hydroxide, choline, deanol, diethanolamine, diethylamine, 2-(diethylamino)ethanol, ethanolamine, ethylenediamine, N-methylglucamine, hydrabamine, 1H-imidazole, lithium, L-lysine, magnesium, 4-(2-hydroxyethyl)morpholine, piperazine, potassium, 1-(2-hydroxyethyl)pyrrolidine, sodium, triethanolamine, tromethamine, and zinc salts. In certain embodiments, contemplated salts of the invention include, but are not limited to, Na, Ca, K, Mg, Zn or other metal salts. In certain embodiments, contemplated salts of the invention include, but are not limited to, 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, 1-ascorbic acid, 1-aspartic acid, benzenesulfonic acid, benzoic acid, (+)-camphoric acid, (+)-camphor-10-sulfonic acid, capric acid (decanoic acid), caproic acid (hexanoic acid), caprylic acid (octanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, d-glucoheptonic acid, d-gluconic acid, d-glucuronic acid, glutamic acid, glutaric acid, glycerophosphoric acid, glycolic acid, hippuric acid, hydrobromic acid, hydrochloric acid, isobutyric acid, lactic acid, lactobionic acid, lauric acid, maleic acid, 1-malic acid, malonic acid, mandelic acid, methanesulfonic acid, naphthalene-1,5-disulfonic acid, naphthalene-2-sulfonic acid, nicotinic acid, nitric acid, oleic acid, oxalic acid, palmitic acid, pamoic acid, phosphoric acid, proprionic acid, 1-pyroglutamic acid, salicylic acid, sebacic acid, stearic acid, succinic acid, sulfuric acid, 1-tartaric acid, thiocyanic acid, p-toluenesulfonic acid, trifluoroacetic acid, and undecylenic acid acid salts.

The pharmaceutically acceptable acid addition salts can also exist as various solvates, such as with water, methanol, ethanol, dimethylformamide, and the like. Mixtures of such solvates can also be prepared. The source of such solvate can be from the solvent of crystallization, inherent in the solvent of preparation or crystallization, or adventitious to such solvent.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

### Definitions

Unless otherwise defined herein, scientific and technical terms used in this application shall have the meanings that are commonly understood by those of ordinary skill in the art. Generally, nomenclature used in connection with, and techniques of, chemistry, cell and tissue culture, molecular biology, cell and cancer biology, neurobiology, neurochemistry, virology, immunology, microbiology, pharmacology, genetics and protein and nucleic acid chemistry, described herein, are those well-known and commonly used in the art.

The methods and techniques of the present disclosure are generally performed, unless otherwise indicated, according to conventional methods well known in the art and as described in various general and more specific references that are cited and discussed throughout this specification. See, e.g. “Principles of Neural Science”, McGraw-Hill Medical, New York, N.Y. (2000); Motulsky, “Intuitive Biostatistics”, Oxford University Press, Inc. (1995); Lodish et al., “Molecular Cell Biology, 4th ed.”, W. H. Freeman & Co., New York (2000); Griffiths et al., “Introduction to Genetic Analysis, 7th ed.”, W. H. Freeman & Co., N.Y. (1999); and Gilbert et al., “Developmental Biology, 6th ed.”, Sinauer Associates, Inc., Sunderland, MA (2000).

Chemistry terms used herein, unless otherwise defined herein, are used according to conventional usage in the art, as exemplified by “The McGraw-Hill Dictionary of Chemical Terms”, Parker S., Ed., McGraw-Hill, San Francisco, C.A. (1985).

All of the above, and any other publications, patents and published patent applications referred to in this application are specifically incorporated by reference herein. In case of conflict, the present specification, including its specific definitions, will control.

The term “agent” is used herein to denote a chemical compound (such as an organic or inorganic compound, a mixture of chemical compounds), a biological macromolecule (such as a nucleic acid, an antibody, including parts thereof as well as humanized, chimeric and human antibodies and monoclonal antibodies, a protein or portion thereof, e.g., a peptide, a lipid, a carbohydrate), or an extract made from biological materials such as bacteria, plants, fungi, or animal (particularly mammalian) cells or tissues. Agents include, for example, agents whose structure is known, and those whose structure is not known.

A “patient,” “subject,” or “individual” are used interchangeably and refer to either a human or a non-human animal. These terms include mammals, such as humans, primates, livestock animals (including bovines, porcines, etc.), companion animals (e.g., canines, felines, etc.) and rodents (e.g., mice and rats).

“Treating” a condition or patient refers to taking steps to obtain beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

The term “preventing” is art-recognized, and when used in relation to a condition, such as a local recurrence (e.g., pain), a disease such as cancer, a syndrome complex such as heart failure or any other medical condition, is well understood in the art, and includes administration of a composition which reduces the frequency of, or delays the onset of, symptoms of a medical condition in a subject relative to a subject which does not receive the composition. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount.

“Administering” or “administration of” a substance, a compound or an agent to a subject can be carried out using one of a variety of methods known to those skilled in the art. For example, a compound or an agent can be administered, intravenously, arterially, intradermally, intramuscularly, intraperitoneally, subcutaneously, ocularly, sublingually, orally (by ingestion), intranasally (by inhalation), intraspinally, intracerebrally, and transdermally (by absorption, e.g., through a skin duct). A compound or agent can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods.

Appropriate methods of administering a substance, a compound or an agent to a subject will also depend, for example, on the age and/or the physical condition of the subject and the chemical and biological properties of the compound or agent (e.g., solubility, digestibility, bioavailability, stability and toxicity). In some embodiments, a compound or an agent is administered orally, e.g., to a subject by ingestion. In some embodiments, the orally administered compound or agent is in an extended release or slow release formulation, or administered using a device for such slow or extended release.

As used herein, the phrase “conjoint administration” refers to any form of administration of two or more different therapeutic agents such that the second agent is administered while the previously administered therapeutic agent is still effective in the body (e.g., the two agents are simultaneously effective in the patient, which may include synergistic effects of the two agents). For example, the different therapeutic compounds can be administered either in the same formulation or in separate formulations, either concomitantly or sequentially. Thus, an individual who receives such treatment can benefit from a combined effect of different therapeutic agents.

A “therapeutically effective amount” or a “therapeutically effective dose” of a drug or agent is an amount of a drug or an agent that, when administered to a subject will have the intended therapeutic effect. The full therapeutic effect does not necessarily occur by administration of one dose, and may occur only after administration of a series of doses. Thus, a therapeutically effective amount may be administered in one or more administrations. The precise effective amount needed for a subject will depend upon, for example, the subject's size, health and age, and the nature and extent of the condition being treated, such as cancer or a viral infection. The skilled worker can readily determine the effective amount for a given situation by routine experimentation.

As used herein, the terms “optional” or “optionally” mean that the subsequently described event or circumstance may occur or may not occur, and that the description includes instances where the event or circumstance occurs as well as instances in which it does not. For example, “optionally substituted alkyl” refers to the alkyl may be substituted as well as where the alkyl is not substituted.

It is understood that substituents and substitution patterns on the compounds of the present invention can be selected by one of ordinary skilled person in the art to result chemically stable compounds which can be readily synthesized by techniques known in the art, as well as those methods set forth below, from readily available starting materials. If a substituent is itself substituted with more than one group, it is understood that these multiple groups may be on the same carbon or on different carbons, so long as a stable structure results.

As used herein, the term “optionally substituted” refers to the replacement of one to six hydrogen radicals in a given structure with the radical of a specified substituent including, but not limited to: hydroxyl, hydroxyalkyl, alkoxy, halogen, alkyl, nitro, silyl, acyl, acyloxy, aryl, heteroaryl, cycloalkyl, heterocycloalkyl, amino, alkylamino, dialkylamino, amido (—C(O)NH₂), carboxyl (—C(O)OH), cyano, haloalkyl, haloalkoxy, —OCO—CH₂—O-alkyl, —OP(O)(O-alkyl)₂ or —CH₂—OP(O)(O-alkyl)₂. Preferably, “optionally substituted” refers to the replacement of one to four hydrogen radicals in a given structure with the substituents mentioned above. More preferably, one to three hydrogen radicals are replaced by the substituents as mentioned above. It is understood that the substituent can be further substituted.

As used herein, the term “alkyl” refers to saturated aliphatic groups, including but not limited to C₁-C₁₀ straight-chain alkyl groups or C₁-C₁₀ branched-chain alkyl groups. Preferably, the “alkyl” group refers to C₁-C₆ straight-chain alkyl groups or C₁-C₆ branched-chain alkyl groups. Most preferably, the “alkyl” group refers to C₁-C₄ straight-chain alkyl groups or C₁-C₄ branched-chain alkyl groups. Examples of “alkyl” include, but are not limited to, methyl, ethyl, 1-propyl, 2-propyl, n-butyl, sec-butyl, tert-butyl, 1-pentyl, 2-pentyl, 3-pentyl, neo-pentyl, 1-hexyl, 2-hexyl, 3-hexyl, 1-heptyl, 2-heptyl, 3-heptyl, 4-heptyl, 1-octyl, 2-octyl, 3-octyl or 4-octyl and the like. The “alkyl” group may be optionally substituted. A “linear” alkyl group refers to a straight-chain alkyl group without a branching point. A “branched” alkyl group refers to an alkyl group having at least one branch point. Examples of branched alkyl groups include, e.g., isopropyl, sec-butyl, and tert-butyl.

Moreover, the term “alkyl” as used throughout the specification, examples, and claims is intended to include both unsubstituted and substituted alkyl groups, the latter of which refers to alkyl moieties having substituents replacing a hydrogen on one or more carbons of the hydrocarbon backbone, including haloalkyl groups such as trifluoromethyl and 2,2,2-trifluoroethyl, etc.

The term “C_{x-y}” or “Cₓ-C_{y}”, when used in conjunction with a chemical moiety, such as, acyl, acyloxy, alkyl, alkenyl, alkynyl, or alkoxy is meant to include groups that contain from x to y carbons in the chain. C₀alkyl indicates a hydrogen where the group is in a terminal position, a bond if internal. A C₁₋₆alkyl group, for example, contains from one to six carbon atoms in the chain.

The term “alkenyl” as used herein means a straight or branched chain hydrocarbon radical containing from 2 to 10 carbons and containing at least one carbon-carbon double bond formed by the removal of two hydrogens. Representative examples of alkenyl include, but are not limited to, ethenyl, 2-propenyl, 2-methyl-2-propenyl, 3-butenyl, 4-pentenyl, 5-hexenyl, 2-heptenyl, 2-methyl-1-heptenyl, and 3-decenyl. The unsaturated bond(s) of the alkenyl group can be located anywhere in the moiety and can have either the (Z) or the (E) configuration about the double bond(s).

The term “alkynyl” as used herein means a straight or branched chain hydrocarbon radical containing from 2 to 10 carbon atoms and containing at least one carbon-carbon triple bond. Representative examples of alkynyl include, but are not limited, to acetylenyl, 1-propynyl, 2-propynyl, 3-butynyl, 2-pentynyl, and 1-butynyl.

The term “acyl” is art-recognized and refers to a group represented by the general formula hydrocarbylC(O)—, preferably alkylC(O)—.

The term “acylamino” is art-recognized and refers to an amino group substituted with an acyl group and may be represented, for example, by the formula hydrocarbylC(O)NH—, preferably alkylC(O)NH—.

The term “acyloxy” is art-recognized and refers to a group represented by the general formula hydrocarbylC(O)O—, preferably alkylC(O)O—.

The term “alkoxy” refers to an alkyl group appended to the parent molecular moiety through an oxygen atom. Representative alkoxy groups include methoxy, ethoxy, propoxy, tert-butoxy and the like.

The term “alkoxyalkyl” refers to an alkyl group substituted with an alkoxy group and may be represented by the general formula -alkyl-O-alkyl.

The term “alkylamino”, as used herein, refers to an amino group substituted with at least one alkyl group. A “dialkylamino” refers to an amino group substituted with two alkyl groups.

The term “alkylthio”, as used herein, refers to a thiol group substituted with an alkyl group and may be represented by the general formula alkylS—.

The term “amide”, as used herein, refers to a group

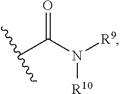

wherein R⁹ and R¹⁰ each independently represent a hydrogen or hydrocarbyl group, or R⁹ and R¹⁰ taken together with the N atom to which they are attached complete a heterocycle having from 4 to 8 atoms in the ring structure. In some embodiments, “amido” refers to the group —C(O)NH₂.

The terms “amine” and “amino” are art-recognized and refer to both unsubstituted and substituted amines and salts thereof, e.g., a moiety that can be represented by

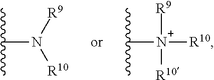

wherein R⁹, R¹⁰, and R^{10′} each independently represent a hydrogen or a hydrocarbyl group, or R⁹ and R¹⁰ taken together with the N atom to which they are attached complete a heterocycle having from 4 to 8 atoms in the ring structure. In some embodiments, “amino” refers to the group —NH₂.

The term “aminoalkyl”, as used herein, refers to an alkyl group substituted with an amino group.

The term “arylalkyl”, as used herein, refers to an alkyl group substituted with an aryl group.

The term “aryl” as used herein include substituted or unsubstituted single-ring aromatic groups in which each atom of the ring is carbon. Preferably the ring is a 5- to 7-membered ring, more preferably a 6-membered ring. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Aryl groups include benzene, naphthalene, phenanthrene, phenol, aniline, and the like.

The term “carbamate” is art-recognized and refers to a group

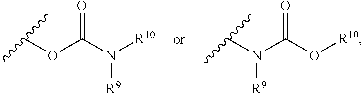

wherein R⁹ and R¹⁰ independently represent hydrogen or a hydrocarbyl group.

The term “carbocyclylalkyl”, as used herein, refers to an alkyl group substituted with a carbocycle group.

The term “carbocycle” includes 5-7 membered monocyclic and 8-12 membered bicyclic rings. Each ring of a bicyclic carbocycle may be selected from saturated, unsaturated and aromatic rings. Carbocycle includes bicyclic molecules in which one, two or three or more atoms are shared between the two rings. The term “fused carbocycle” refers to a bicyclic carbocycle in which each of the rings shares two adjacent atoms with the other ring. Each ring of a fused carbocycle may be selected from saturated, unsaturated and aromatic rings. In an exemplary embodiment, an aromatic ring, e.g., phenyl, may be fused to a saturated or unsaturated ring, e.g., cyclohexane, cyclopentane, or cyclohexene. Any combination of saturated, unsaturated and aromatic bicyclic rings, as valence permits, is included in the definition of carbocyclic. Exemplary “carbocycles” include cyclopentane, cyclohexane, bicyclo[2.2.1]heptane, 1,5-cyclooctadiene, 1,2,3,4-tetrahydronaphthalene, bicyclo[4.2.0]oct-3-ene, naphthalene and adamantane. Exemplary fused carbocycles include decalin, naphthalene, 1,2,3,4-tetrahydronaphthalene, bicyclo[4.2.0]octane, 4,5,6,7-tetrahydro-1H-indene and bicyclo[4.1.0]hept-3-ene. “Carbocycles” may be substituted at any one or more positions capable of bearing a hydrogen atom.

The term “carbocyclylalkyl”, as used herein, refers to an alkyl group substituted with a carbocycle group.

The term “carbonate” is art-recognized and refers to a group —OCO₂—.

The term “carboxy” or “carboxyl”, as used herein, refers to a group represented by the formula —CO₂H.

The term “cycloalkyl” includes substituted or unsubstituted non-aromatic single ring structures, preferably 4- to 8-membered rings, more preferably 4- to 6-membered rings. The term “cycloalkyl” also includes polycyclic ring systems having two or more cyclic rings in which two or more carbons are common to two adjoining rings wherein at least one of the rings is cycloalkyl and the substituent (e.g., R¹⁰⁰) is attached to the cycloalkyl ring, e.g., the other cyclic rings can be cycloalkyls, cycloalkenyls, cycloalkynyls, aryls, heteroaryls, and/or heterocyclyls. Cycloalkyl groups include, e.g., cyclopropyl, cyclobutyl, cyclopentyl, and cyclohexyl.

The term “ester”, as used herein, refers to a group —C(O)OR⁹ wherein R⁹ represents a hydrocarbyl group.

The term “ether”, as used herein, refers to a hydrocarbyl group linked through an oxygen to another hydrocarbyl group. Accordingly, an ether substituent of a hydrocarbyl group may be hydrocarbyl-O—. Ethers may be either symmetrical or unsymmetrical.

Examples of ethers include, but are not limited to, heterocycle-O-heterocycle and aryl-O-heterocycle. Ethers include “alkoxyalkyl” groups, which may be represented by the general formula alkyl-O-alkyl.

The terms “halo” and “halogen” as used herein means halogen and includes chloro, fluoro, bromo, and iodo.

The terms “heteroaralkyl” and “heteroarylalkyl”, as used herein, refers to an alkyl group substituted with a heteroaryl group.

The term “heteroaryl” includes substituted or unsubstituted aromatic single ring structures, preferably 5- to 7-membered rings, more preferably 5- to 6-membered rings, whose ring structures include at least one heteroatom, preferably one to four heteroatoms, more preferably one or two heteroatoms. The term “heteroaryl” also includes polycyclic ring systems having two or more cyclic rings in which two or more carbons are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, oxazole, thiazole, pyrazole, pyridine, pyrazine, pyridazine, and pyrimidine, and the like.

The term “heteroatom” as used herein means an atom of any element other than carbon or hydrogen. Preferred heteroatoms are nitrogen, oxygen, and sulfur.

The terms “heterocycloalkylalkyl” and “heterocyclylalkyl”, as used herein, refers to an alkyl group substituted with a heterocycle group.

The terms “heterocycloalkyl,” “heterocyclyl”, “heterocycle”, and “heterocyclic” refer to substituted or unsubstituted non-aromatic ring structures, preferably 3- to 10-membered rings, more preferably 3- to 7-membered rings, whose ring structures include at least one heteroatom, preferably one to four heteroatoms, more preferably one or two heteroatoms. The terms “heterocycloalkyl,” “heterocyclyl”, “heterocycle”, and “heterocyclic” also include polycyclic ring systems having two or more cyclic rings in which two or more carbons are common to two adjoining rings wherein at least one of the rings is heterocyclic, e.g., the other cyclic rings can be cycloalkyls, cycloalkenyls, cycloalkynyls, aryls, heteroaryls, and/or heterocyclyls. Heterocycloalkyl groups include, for example, piperidine, piperazine, pyrrolidine, morpholine, lactones, lactams, and the like.

The term “hydrocarbyl”, as used herein, refers to a group that is bonded through a carbon atom that does not have a ═O or ═S substituent, and typically has at least one carbon-hydrogen bond and a primarily carbon backbone, but may optionally include heteroatoms. Thus, groups like methyl, ethoxyethyl, 2-pyridyl, and even trifluoromethyl are considered to be hydrocarbyl for the purposes of this application, but substituents such as acetyl (which has a ═O substituent on the linking carbon) and ethoxy (which is linked through oxygen, not carbon) are not. Hydrocarbyl groups include, but are not limited to aryl, heteroaryl, carbocycle, heterocycle, alkyl, alkenyl, alkynyl, and combinations thereof.

The term “hydroxyalkyl”, as used herein, refers to an alkyl group substituted with a hydroxy group.

The term “lower” when used in conjunction with a chemical moiety, such as acyl, acyloxy, alkyl, alkenyl, alkynyl, or alkoxy is meant to include groups where there are ten or fewer atoms in the substituent, preferably six or fewer. A “lower alkyl”, for example, refers to an alkyl group that contains ten or fewer carbon atoms, preferably six or fewer. In certain embodiments, acyl, acyloxy, alkyl, alkenyl, alkynyl, or alkoxy substituents defined herein are respectively lower acyl, lower acyloxy, lower alkyl, lower alkenyl, lower alkynyl, or lower alkoxy, whether they appear alone or in combination with other substituents, such as in the recitations hydroxyalkyl and aralkyl (in which case, for example, the atoms within the aryl group are not counted when counting the carbon atoms in the alkyl substituent).

The terms “polycyclyl”, “polycycle”, and “polycyclic” refer to two or more rings (e.g., cycloalkyls, cycloalkenyls, cycloalkynyls, aryls, heteroaryls, and/or heterocyclyls) in which two or more atoms are common to two adjoining rings, e.g., the rings are “fused rings”. Each of the rings of the polycycle can be substituted or unsubstituted. In certain embodiments, each ring of the polycycle contains from 3 to 10 atoms in the ring, preferably from 5 to 7.

The term “sulfate” is art-recognized and refers to the group —OSO₃H, or a pharmaceutically acceptable salt thereof.

The term “sulfonamide” is art-recognized and refers to the group represented by the general formulae

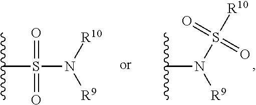

wherein R⁹ and R¹⁰ independently represents hydrogen or hydrocarbyl.

The term “sulfoxide” is art-recognized and refers to the group —S(O)—.

The term “sulfonate” is art-recognized and refers to the group SO₃H, or a pharmaceutically acceptable salt thereof.

The term “sulfone” is art-recognized and refers to the group —S(O)₂—.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate.

The term “thioalkyl”, as used herein, refers to an alkyl group substituted with a thiol group.

The term “thioester”, as used herein, refers to a group —C(O)SR⁹ or —SC(O)R⁹ wherein R⁹ represents a hydrocarbyl.

The term “thioether”, as used herein, is equivalent to an ether, wherein the oxygen is replaced with a sulfur.

The term “urea” is art-recognized and may be represented by the general formula

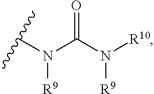

wherein R⁹ and R¹⁰ independently represent hydrogen or a hydrocarbyl.

The term “modulate” as used herein includes the inhibition or suppression of a function or activity (such as cell proliferation) as well as the enhancement of a function or activity.

The phrase “pharmaceutically acceptable” is art-recognized. In certain embodiments, the term includes compositions, excipients, adjuvants, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable salt” or “salt” is used herein to refer to an acid addition salt or a basic addition salt which is suitable for or compatible with the treatment of patients.

The term “pharmaceutically acceptable acid addition salt” as used herein means any non-toxic organic or inorganic salt of any base compounds disclosed herein. Illustrative inorganic acids which form suitable salts include hydrochloric, hydrobromic, sulfuric and phosphoric acids, as well as metal salts such as sodium monohydrogen orthophosphate and potassium hydrogen sulfate. Illustrative organic acids that form suitable salts include mono-, di-, and tricarboxylic acids such as glycolic, lactic, pyruvic, malonic, succinic, glutaric, fumaric, malic, tartaric, citric, ascorbic, maleic, benzoic, phenylacetic, cinnamic and salicylic acids, as well as sulfonic acids such as p-toluene sulfonic and methanesulfonic acids. Either the mono or di-acid salts can be formed, and such salts may exist in either a hydrated, solvated or substantially anhydrous form. In general, the acid addition salts of compounds of Formula I are more soluble in water and various hydrophilic organic solvents, and generally demonstrate higher melting points in comparison to their free base forms. The selection of the appropriate salt will be known to one skilled in the art. Other non-pharmaceutically acceptable salts, e.g., oxalates, may be used, for example, in the isolation of compounds of Formula I for laboratory use, or for subsequent conversion to a pharmaceutically acceptable acid addition salt.

The term “pharmaceutically acceptable basic addition salt” as used herein means any non-toxic organic or inorganic base addition salt of any acid compounds disclosed herein. Illustrative inorganic bases which form suitable salts include lithium, sodium, potassium, calcium, magnesium, or barium hydroxide. Illustrative organic bases which form suitable salts include aliphatic, alicyclic, or aromatic organic amines such as methylamine, trimethylamine and picoline or ammonia. The selection of the appropriate salt will be known to a person skilled in the art.

In certain embodiments, the lipidoid compositions (e.g., nanoparticles) useful in the methods and compositions of this disclosure have at least one stereogenic center in their structure. This stereogenic center may be present in a R or a S configuration, said R and S notation is used in correspondence with the rules described in Pure Appl. Chem. (1976), 45, 11-30. The disclosure contemplates all stereoisomeric forms such as enantiomeric and diastereoisomeric forms of the compounds, salts, prodrugs or mixtures thereof (including all possible mixtures of stereoisomers). See, e.g., WO 01/062726.

Some of the lipidoid compositions (e.g., nanoparticles) may also comprise chemical compound which exist in tautomeric forms. Such forms, although not explicitly indicated in the formulae described herein, are intended to be included within the scope of the present disclosure.

The phrase “pharmaceutically acceptable carrier” as used herein means a pharmaceutically acceptable material, composition or vehicle, such as a liquid or solid filter, diluent, excipient, solvent or encapsulating material useful for formulating a drug for medicinal or therapeutic use.

### EXAMPLES

The invention now being generally described, it will be more readily understood by reference to the following examples which are included merely for purposes of illustration of certain aspects and embodiments of the present invention, and are not intended to limit the invention.

### Example 1. Synthesis and Characterization of Series 100 and 300 Lipidoids

In the following schemes, stereochemistry of the amino acid headgroup has been omitted for clarity. In Example 1, the lipidoid syntheses use L-amino acids in the headgroup.

### Synthesis of Compound 100 and Compound 300

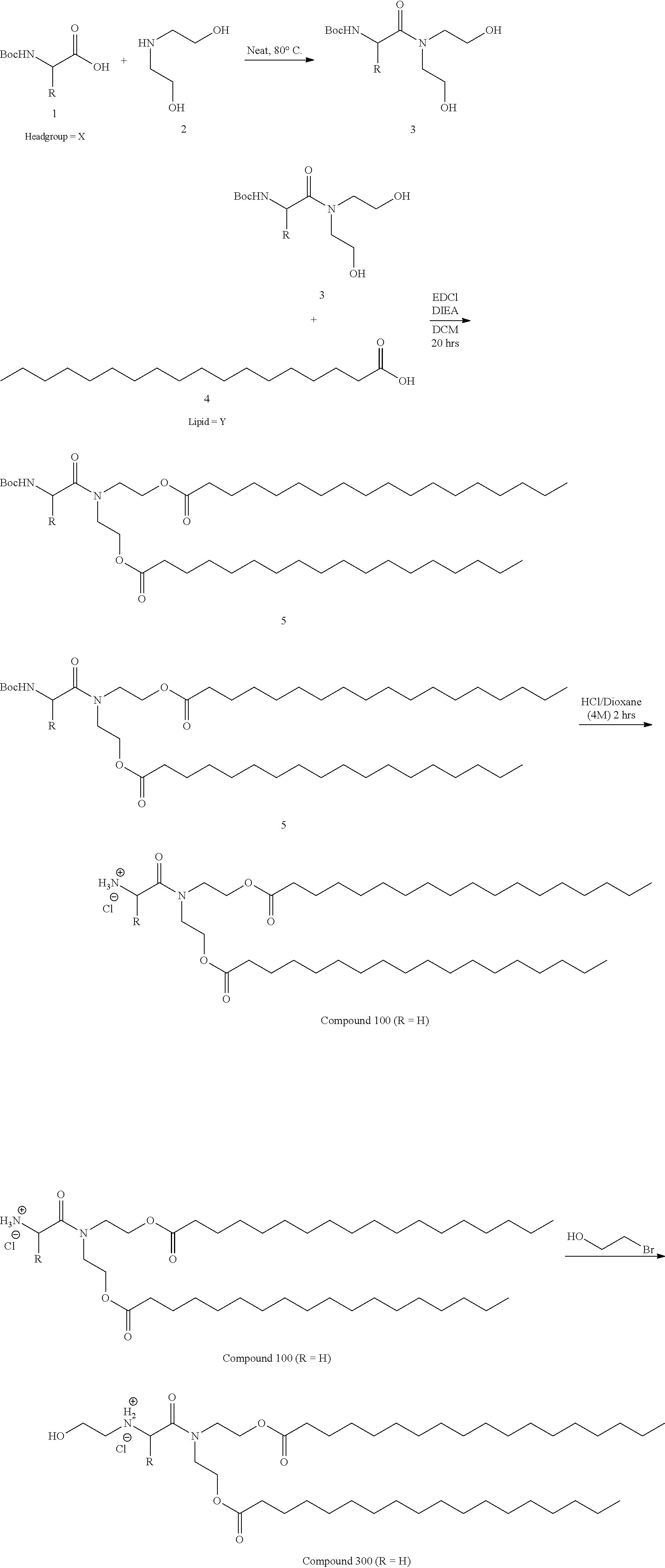

### Representative Synthesis of Compound 3:

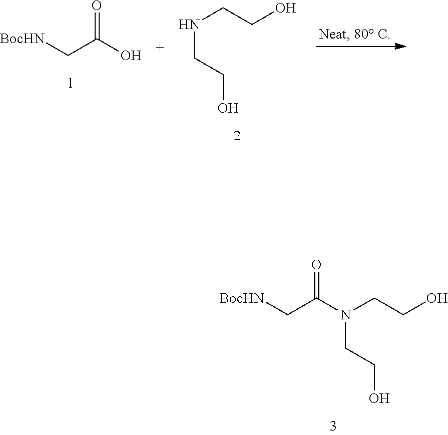 [SMILES: CC(C)(C)OC(=O)NCC(=O)O ; OCCNCCO ; CC(C)(C)OC(=O)NCC(=O)N(CCO)CCO]

A mixture of Compound 1 (5 g, 26.43 mmol, 3.91 mL, 1 eq.) and Compound 2 (3.06 g, 29.07 mmol, 2.81 mL, 1.1 eq.) was stirred at 20° C. for 16 hours. LCMS showed Compound 1 was consumed completely and desired MS was detected. The mixture was concentrated to give a residue. The residue was purified by column chromatography (SiO₂, DCM: MeOH=20:1 to 5:1) to give Compound 3 (4.8 g, 17.93 mmol, 67.86% yield, 98% purity) as white solid. LCMS [M+1]⁺=263.3.

¹H NMR (400 MHz, DMSO-d₆) δ=6.63-6.55 (m, 1H), 4.87-4.82 (m, 1H), 4.66 (t, J=5.2 Hz, 1H), 3.84 (br d, J=5.6 Hz, 2H), 3.56-3.49 (m, 2H), 3.47-3.42 (m, 2H), 3.37-3.33 (m, 4H), 1.43-1.33 (m, 9H).

### Representative Lipid Addition of Diethanolamine Functionalized Amino Acid Headgroups:

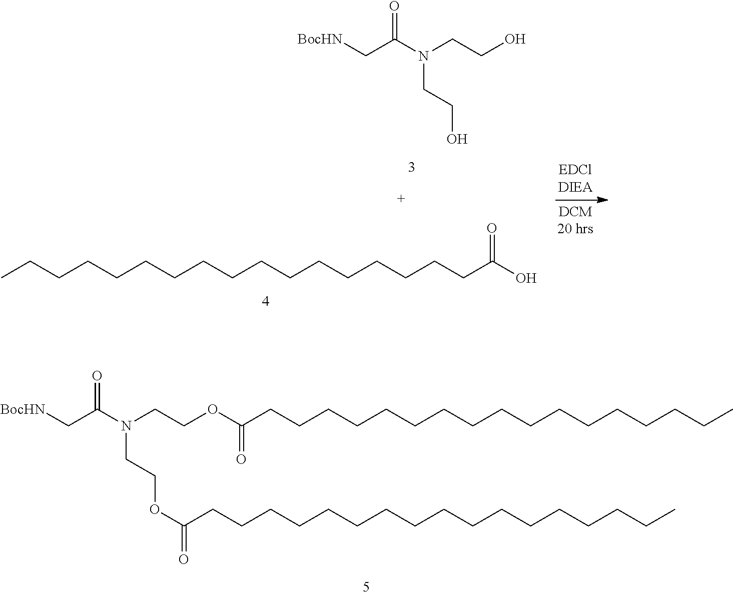 [SMILES: CC(C)(C)OC(=O)NCC(=O)N(CCO)CCO ; CCCCCCCCCCCCCCCCCC(=O)O ; CCCCCCCCCCCCCCCCCC(=O)OCCN(CCOC(=O)CCCCCCCCCCCCCCCCC)C(=O)CNC(=O)OC(C)(C)C]

A mixture of Compound 3 (0.5 g, 1.91 mmol, 1 eq.), Compound 4 (1.19 g, 4.19 mmol, 1.41 mL, 2.2 eq.), EDCI (1.10 g, 5.72 mmol, 3 eq.), DIEA (739.09 mg, 5.72 mmol, 996.08 μL, 3 eq.) and DMAP (116.44 mg, 953.10 μmol, 0.5 eq.) in DCM (5 mL) was degassed and purged with N₂ for 3 times, and the mixture was stirred at 25° C. for 16 hours under N₂ atmosphere. TLC (Petroleum ether:Ethyl acetate=5:1, R_{f}=0.45) indicated Compound 3 was consumed completely and many new spots formed. The reaction mixture was partitioned between CH₂Cl₂ (15 mL) and H₂O (5 mL). The organic phase was separated, dried over Na₂SO₄, filtered and concentrated under reduced pressure to give a residue. The residue was purified by column chromatography (SiO₂, Petroleum ether:Ethyl acetate=80:1 to 1:100) to give Compound 5 (1.16 g, 1.46 mmol, 76.52% yield, 100% purity) as white solid. LCMS [M+1]⁺=795.7.

¹H NMR (400 MHz, chloroform-d) δ=5.52-5.36 (m, 1H), 4.28-4.12 (m, 4H), 4.04 (br d, J=3.6 Hz, 2H), 3.69-3.45 (m, 4H), 2.35-2.26 (m, 4H), 1.65-1.55 (m, 4H), 1.45 (s, 9H), 1.26 (s, 54H), 0.91-0.85 (m, 6H).

### Representative Procedure for Deprotection of Lipid Precursors:

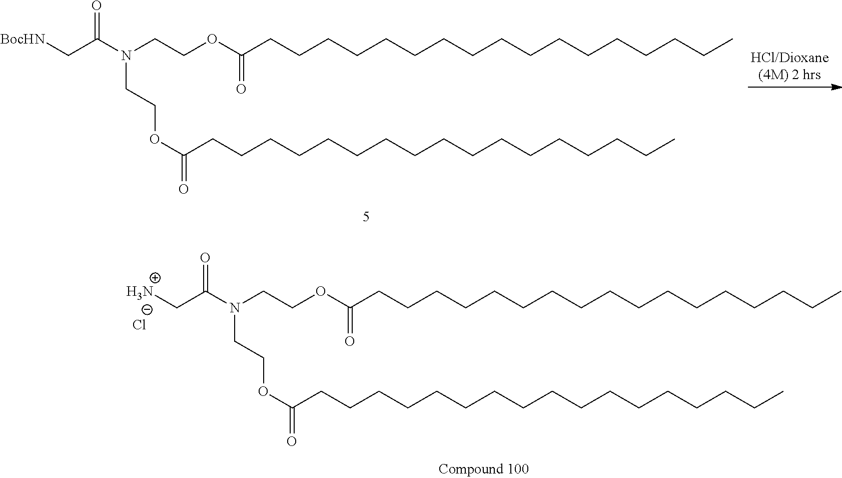 [SMILES: CCCCCCCCCCCCCCCCCC(=O)OCCN(CCOC(=O)CCCCCCCCCCCCCCCCC)C(=O)CNC(=O)OC(C)(C)C ; [Cl-] ; CCCCCCCCCCCCCCCCCC(=O)OCCN(CCOC(=O)CCCCCCCCCCCCCCCCC)C(=O)C[NH3+]]

a solution of Compound 5 (200 mg, 319.03 μmol, 1 eq.) in EtOAc (15 mL) was added HCl/EtOAc (15 mL) at 20° C., and the mixture was stirred at 20° C. for 2 hours. The mixture was concentrated under reduced pressure to give crude product. The crude product was triturated with DCM (3 mL) at 20° C. for 30 min, and the filter cake was dried and lyophilized to give Compound 100 (110 mg, 150.36 μmol, 80.40% yield, HCl) as white solid. LCMS [M+1]⁺=695.6.

¹H NMR (400 MHz, methanol-d₄) δ=4.25 (td, J=5.6, 7.6 Hz, 4H), 3.99 (s, 2H), 3.78-3.60 (m, 4H), 2.42-2.25 (m, 4H), 1.61 (br d, J=4.4 Hz, 4H), 1.29 (s, 56H), 0.96-0.84 (m, 6H).

### Representative Synthesis of Hydroxyethyl Functionalized Lipids:

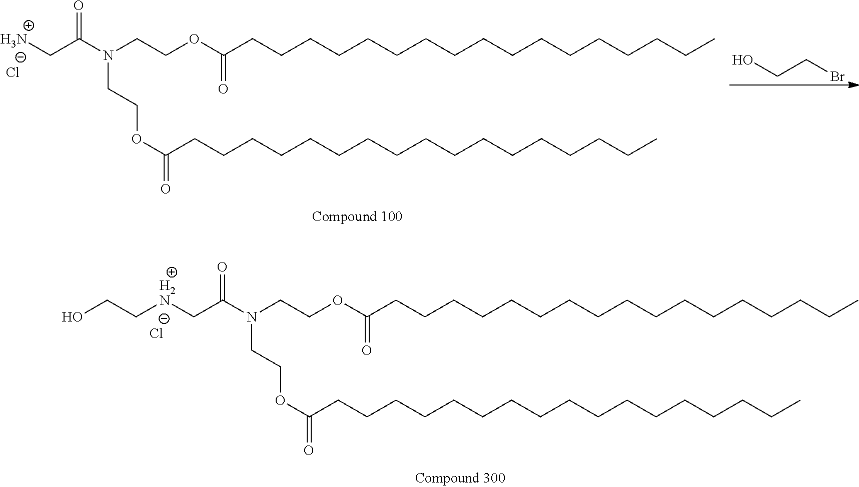

To a solution of Compound 100 (400 mg, 575.45 μmol, 1 eq.) in ACN (20 mL) was added TEA (174.69 mg, 1.73 mmol, 240.29 μL, 3 eq.) and Bromoethanol (143.82 mg, 1.15 mmol, 81.58 μL, 2 eq.). The mixture was stirred at 80° C. for 48 hours. LC-MS showed Gly-Stea was consumed completely and desired MS was detected. The reaction mixture was concentrated under reduced pressure to give a residue. The residue was purified by prep-TLC (SiO₂, DCM:MeOH=5:1) to give Compound 300 (100 mg, 135.29 μmol, 23.51% yield) as white solid. To a solution of Compound 300 (80 mg, 108.23 μmol, 1 eq.) in dioxane (2.5 mL) was added HCl/dioxane (0.5 M, 432.92 μL, 2 eq.). The mixture was stirred at 20° C. for 2 hours. The reaction mixture was concentrated and lyophilized to give Compound 300 (103 mg, HCl) as white solid. LCMS [M+1]⁺=739.7.

¹H NMR (400 MHz, DMSO-d₆) δ=8.69 (br s, 2H), 5.18 (br t, J=4.0 Hz, 1H), 4.23-4.10 (m, 4H), 4.08 (br d, J=2.4 Hz, 2H), 3.67 (br d, J=4.4 Hz, 2H), 3.58 (br s, 4H), 3.00 (br s, 2H), 2.35-2.24 (m, 4H), 1.50 (br s, 4H), 1.23 (br s, 56H), 0.91-0.81 (m, 6H).

### Characterization Data for all Series 100 and 300 Lipids:

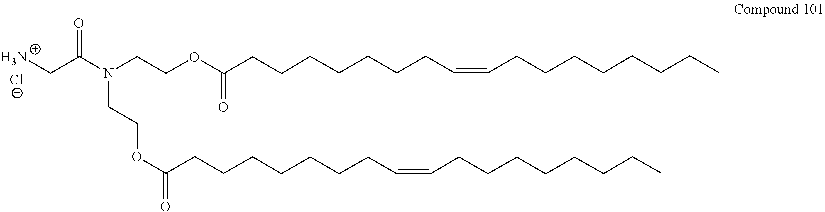 [SMILES: [Cl-] ; CCCCCCCC/C=C\CCCCCCCC(=O)OCCN(CCOC(=O)CCCCCCC/C=C\CCCCCCCC)C(=O)C[NH3+]]

LCMS [M+1]⁺=691.6. ¹H NMR (400 MHz, chloroform-d) δ=8.54 (br s, 3H), 5.47-5.23 (m, 4H), 4.27-4.13 (m, 4H), 3.80-3.50 (m, 4H), 2.43-2.25 (m, 4H), 2.01 (d, J=5.6 Hz, 8H), 1.75 (s, 8H), 1.29 (d, J=12.4 Hz, 40H), 0.96-0.81 (m, 6H).

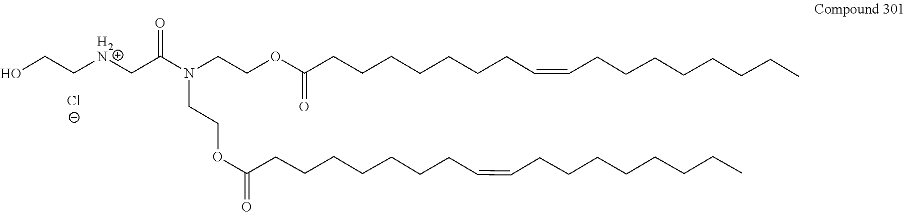

LCMS [M+1]⁺=735.7. ¹H NMR (400 MHz, DMSO-d₆) δ=8.67-8.70 (m, 2H), 5.28-5.36 (m, 4 H), 5.16-5.19 (t, J=5.2 Hz, 1H), 4.12-4.18 (dt, J=16.6, 5.2 Hz, 4H), 4.06 (s, 2H), 3.65-3.69 (m, 2H), 3.56-3.60 (m, 4H), 2.99-3.01 (t, J=5.2 Hz, 2H), 2.25-2.33 (m, 4H), 1.93-2.02 (m, 8H), 1.43-1.51 (m, 4H), 1.09-1.37 (m, 40H), 0.83-0.87 (m, 6H).

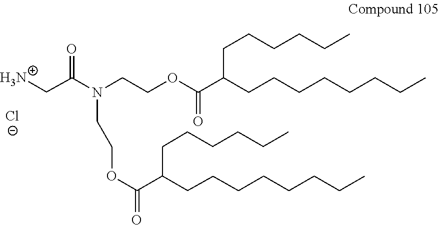 [SMILES: [Cl-] ; CCCCCCCCC(CCCCCC)C(=O)OCCN(CCOC(=O)C(CCCCCC)CCCCCCCC)C(=O)C[NH3+]]

LCMS [M+1]⁺=639.6. ¹H NMR (400 MHz, chloroform-d) δ=8.75-8.35 (m, 3H), 4.26 (br t, J=5.6 Hz, 2H), 4.21 (br t, J=5.6 Hz, 2H), 4.13 (br s, 2H), 3.73-3.54 (m, 4H), 2.29 (br s, 2H), 1.63-1.52 (m, 4H), 1.50-1.41 (m, 4H), 1.35-1.13 (m, 40H), 0.88 (t, J=6.8 Hz, 12H).

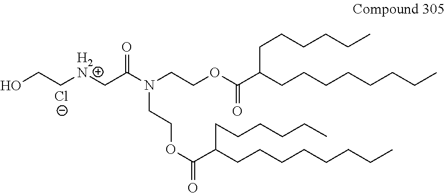

LCMS [M+1]⁺=683.6. ¹H NMR (400 MHz, DMSO-d₆) δ=8.75 (d, J=1.2 Hz, 2H), 5.25-5.14 (m, 1H), 4.18 (t, J=5.6 Hz, 2H), 4.15-4.03 (m, 4H), 3.68 (s, 2H), 3.63-3.50 (m, 4H), 3.01 (s, 2H), 2.38-2.26 (m, 2H), 1.52-1.37 (m, 8H), 1.23 (s, 40H), 0.85 (t, J=6.4 Hz, 12H).

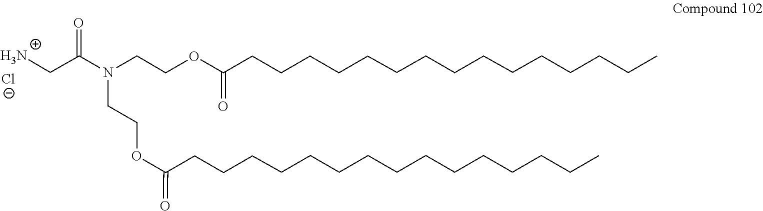 [SMILES: [Cl-] ; CCCCCCCCCCCCCCCC(=O)OCCN(CCOC(=O)CCCCCCCCCCCCCCC)C(=O)C[NH3+]]

LCMS [M+1]⁺=639.6. ¹H NMR (400 MHz, methanol-d₄) δ=4.34-4.16 (m, 4H), 4.00 (s, 2H), 3.76-3.57 (m, 4H), 2.40-2.22 (m, 4H), 1.61 (br d, J=4.4 Hz, 4H), 1.37-1.18 (m, 48H), 0.90 (t, J=6.8 Hz, 6H).

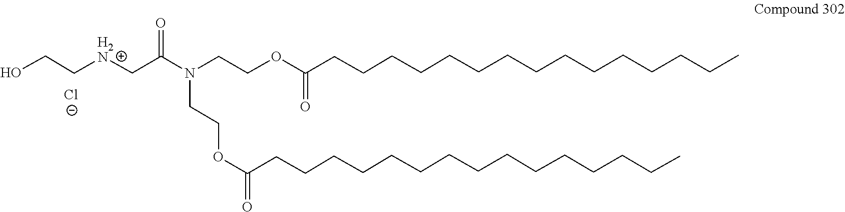

LCMS [M+1]⁺=683.6. ¹H NMR (400 MHz, DMSO-d₆) δ=8.87-8.67 (m, 2H), 5.27-5.12 (m, 1H), 4.24-4.00 (m, 6H), 3.73-3.51 (m, 6H), 3.08-2.94 (m, 2H), 2.38-2.21 (m, 4H), 1.56-1.47 (m, 4H), 1.35-1.16 (m, 48H), 0.92-0.79 (m, 6H).

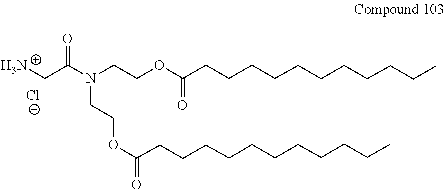 [SMILES: CCCCCCCCCCCC(=O)OCCN(CCOC(=O)CCCCCCCCCCC)C(=O)C[NH3+] ; [Cl-]]

LCMS [M+1]⁺=527.4. ¹H NMR (400 MHz, DMSO-d₆) δ=8.13-7.90 (m, 3H), 4.27-4.10 (m, 4H), 3.88 (s, 2H), 3.58 (br d, J=3.2 Hz, 4H), 2.37-2.20 (m, 4H), 1.50 (br s, 4H), 1.24 (br s, 32H), 0.85 (br t, J=6.4 Hz, 6H).

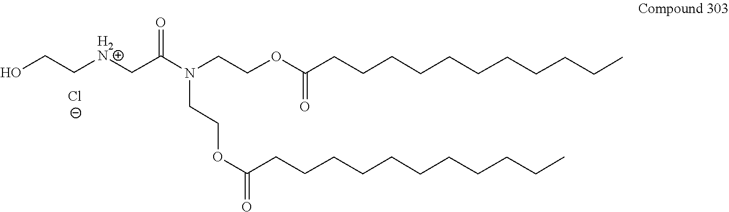

LCMS [M+1]⁺=571.5. ¹H NMR (400 MHz, DMSO-d₆) δ=9.05-8.23 (m, 2H), 5.24-5.08 (m, 1H), 4.31-3.94 (m, 6H), 3.76-3.43 (m, 6H), 3.07-2.92 (m, 2H), 2.37-2.22 (m, 4H), 1.56-1.44 (m, 4H), 1.37-1.01 (m, 32H), 0.94-0.76 (m, 6H).

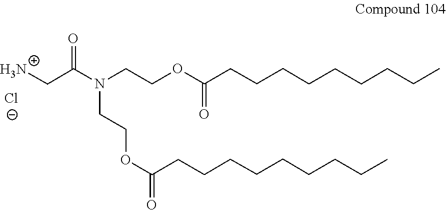 [SMILES: [Cl-] ; CCCCCCCCCC(=O)OCCN(CCOC(=O)CCCCCCCCC)C(=O)C[NH3+]]

LCMS [M+1]⁺=527.4, LCMS [2M+1]⁺=471.4. ¹H NMR (400 MHz, DMSO-d₆) δ=8.12 (s, 3H), 4.06-4.24 (t, J=18.8, 5.42 Hz, 4H), 3.87 (s, 2H), 3.57-3.58 (m, 4H), 2.26-2.50 (m, 4H), 1.49-1.52 (m, 4H), 1.24 (s, 24H), 0.84-0.87 (t, J=6.8 Hz, 6H).

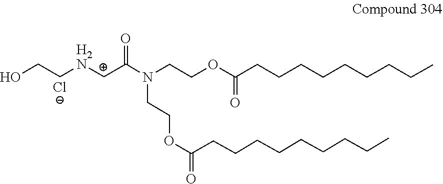

LCMS [M+1]⁺=515.5, LCMS [2M+1]⁺=1029.8. ¹H NMR (400 MHz, DMSO-d₆) δ=8.98-8.53 (m, 2H), 5.23-5.14 (m, 1H), 4.26-4.10 (m, 4H), 4.09-3.98 (m, 2H), 3.76-3.62 (m, 2H), 3.61-3.45 (m, 4H), 3.00 (br t, J=5.2 Hz, 2H), 2.30 (td, J=7.4, 18.8 Hz, 4H), 1.51 (br s, 4H), 1.24 (br s, 24H), 0.85 (br t, J=6.4 Hz, 6H).

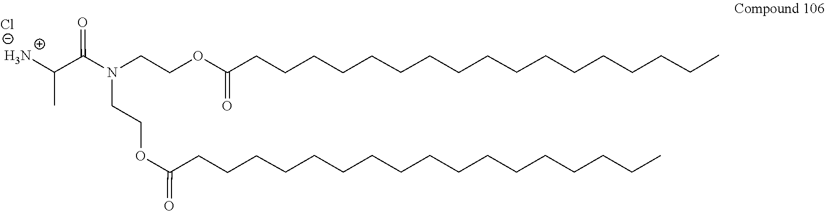 [SMILES: [Cl-] ; CCCCCCCCCCCCCCCCCC(=O)OCCN(CCOC(=O)CCCCCCCCCCCCCCCCC)C(=O)C(C)[NH3+]]

LCMS [M+1]⁺=709.6. ¹H NMR (400 MHz, chloroform-d) δ=8.34 (br s, 3H), 4.75-4.56 (m, 1H), 4.43-4.12 (m, 4H), 3.99-3.82 (m, 1H), 3.67 (br d, J=3.2 Hz, 2H), 3.55-3.36 (m, 1H), 2.39-2.24 (m, 4H), 1.60 (br s, 7H), 1.26 (s, 56H), 0.95-0.83 (m, 6H).

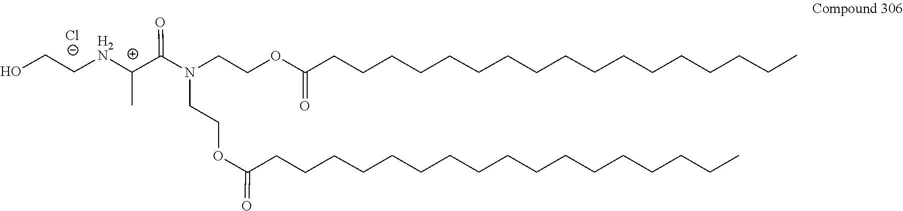

LCMS [M+1]⁺=753.7. ¹H NMR (400 MHz, DMSO-d₆) δ=9.03-8.88 (m, 1H), 8.78-8.61 (m, 1H), 5.20 (t, J=4.4 Hz, 1H), 4.38 (br d, J=3.6 Hz, 1H), 4.27-4.08 (m, 4H), 3.82-3.69 (m, 1H), 3.69-3.56 (m, 4H), 3.54-3.44 (m, 1H), 3.03-2.80 (m, 2H), 2.28 (td, J=7.6, 18.7 Hz, 4H), 1.50 (br d, J=6.0 Hz, 4H), 1.38 (br d, J=6.4 Hz, 3H), 1.24 (s, 56H), 0.91-0.81 (m, 6H).

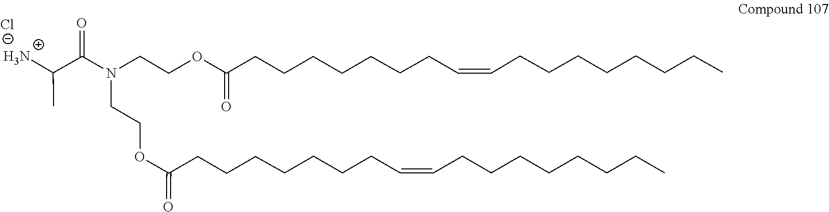 [SMILES: [Cl-] ; CCCCCCCC/C=C\CCCCCCCC(=O)OCCN(CCOC(=O)CCCCCCC/C=C\CCCCCCCC)C(=O)C(C)[NH3+]]

LCMS [M+1]⁺=705.5. ¹H NMR (400 MHz, chloroform-d) δ=9.16-7.77 (m, 3H), 5.45-5.27 (m, 4H), 4.62 (br d, J=6.8 Hz, 1H), 4.32 (br d, J=8.4 Hz, 2H), 4.21 (br t, J=5.2 Hz, 2H), 3.97-3.80 (m, 1H), 3.67 (br s, 2H), 3.52-3.37 (m, 1H), 2.41-2.23 (m, 4H), 2.12-1.93 (m, 8H), 1.59 (br d, J=6.4 Hz, 7H), 1.29 (br d, J=11.2 Hz, 40H), 0.89 (t, J=6.8 Hz, 6H).

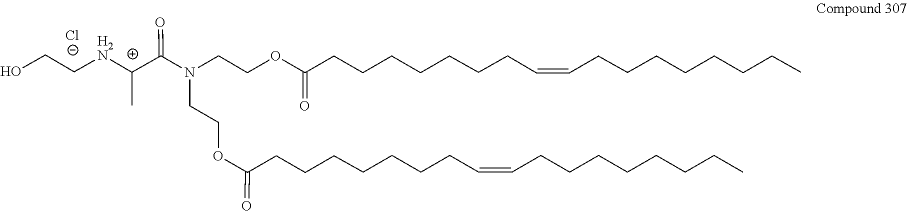

LCMS [M+1]⁺=749.7. ¹H NMR (400 MHz, DMSO-d₆) δ=9.64-8.20 (m, 2H), 5.32 (br t, J=4.8 Hz, 4H), 5.26-5.17 (m, 1H), 4.38 (br d, J=6.8 Hz, 1H), 4.16 (td, J=5.0, 9.6 Hz, 4H), 3.71 (br s, 1H), 3.69-3.55 (m, 4H), 3.53-3.42 (m, 1H), 3.04-2.78 (m, 2H), 2.28 (td, J=7.4, 19.2 Hz, 4H), 1.97 (br d, J=5.6 Hz, 8H), 1.50 (br d, J=6.4 Hz, 4H), 1.38 (br d, J=6.8 Hz, 3H), 1.24 (br s, 40H), 0.85 (br t, J=6.8 Hz, 6H).

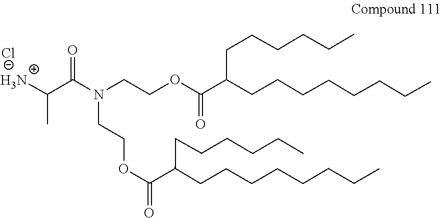 [SMILES: [Cl-] ; CCCCCCCCC(CCCCCC)C(=O)OCCN(CCOC(=O)C(CCCCCC)CCCCCCCC)C(=O)C(C)[NH3+]]

LCMS [M+1]⁺=653.6. ¹H NMR (400 MHz, DMSO-d₆) δ=8.22 (br s, 3H), 4.29 (br d, J=6.8 Hz, 1H), 4.24-4.05 (m, 4H), 3.85-3.64 (m, 2H), 3.63-3.53 (m, 1H), 3.44-3.36 (m, 1H), 2.36-2.24 (m, 2H), 1.45-1.45 (m, 1H), 1.48 (br s, 3H), 1.41 (br d, J=4.4 Hz, 4H), 1.32 (br d, J=6.5 Hz, 3H), 1.22 (br s, 40H), 0.85 (br t, J=6.4 Hz, 12H).

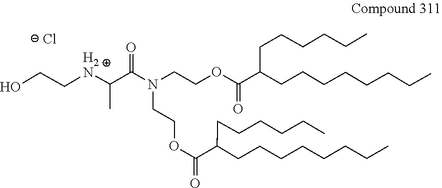

LCMS [M+1]⁺=697.7. ¹H NMR (400 MHz, DMSO-d₆) δ=9.17-8.84 (m, 1H), 8.83-8.49 (m, 1H), 5.20 (br s, 1H), 4.38 (br d, J=5.6 Hz, 1H), 4.26-4.03 (m, 4H), 3.79-3.70 (m, 1H), 3.70-3.56 (m, 4H), 3.53-3.40 (m, 1H), 3.04-2.91 (m, 1H), 2.91-2.80 (m, 1H), 2.29 (br d, J=6.4 Hz, 2H), 1.57-1.39 (m, 8H), 1.37 (br d, J=6.4 Hz, 3H), 1.22 (br s, 40H), 1.17-1.13 (m, 2H), 0.85 (t, J=6.4 Hz, 12H).

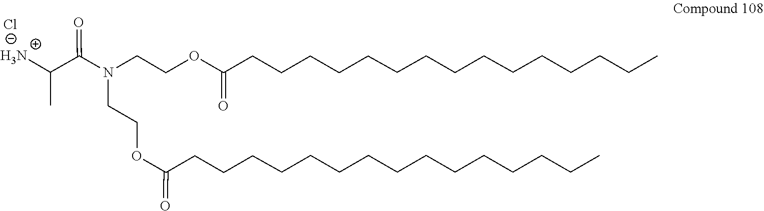 [SMILES: [Cl-] ; CCCCCCCCCCCCCCCC(=O)OCCN(CCOC(=O)CCCCCCCCCCCCCCC)C(=O)C(C)[NH3+]]

LCMS [M+1]⁺=653.6. ¹H NMR (400 MHz, chloroform-d) δ=8.33 (br s, 3H), 4.65 (br s, 1H), 4.33 (br s, 2H), 4.21 (br s, 2H), 3.89 (br d, J=12.4 Hz, 1H), 3.67 (br s, 2H), 3.53-3.38 (m, 1H), 2.31 (td, J=7.6, 14.4 Hz, 4H), 1.69-1.49 (m, 7H), 1.26 (s, 48H), 0.89 (t, J=6.8 Hz, 6H).

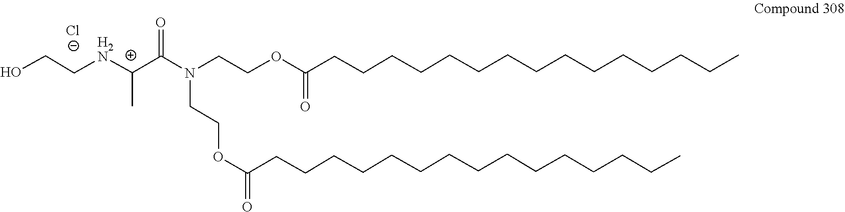

LCMS [M+1]⁺=697.6. ¹H NMR (400 MHz, DMSO-d₆) δ=9.47-9.01 (m, 1H), 8.92-8.51 (m, 1H), 5.22 (br t, J=4.4 Hz, 1H), 4.38 (br d, J=6.4 Hz, 1H), 4.24-4.07 (m, 4H), 3.80-3.69 (m, 1H), 3.69-3.55 (m, 4H), 3.54-3.42 (m, 1H), 3.04-2.91 (m, 1H), 2.89-2.81 (m, 1H), 2.28 (td, J=7.2, 19.0 Hz, 4H), 1.50 (br d, J=5.6 Hz, 4H), 1.38 (br d, J=6.4 Hz, 3H), 1.23 (s, 48H), 0.85 (br t, J=6.4 Hz, 6H).

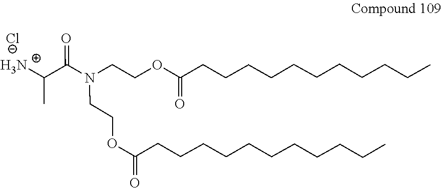 [SMILES: [Cl-] ; CCCCCCCCCCCC(=O)OCCN(CCOC(=O)CCCCCCCCCCC)C(=O)C(C)[NH3+]]

LCMS [M+1]⁺=541.4. ¹H NMR (400 MHz, chloroform-d) δ=8.62-8.09 (m, 3H), 4.65 (br s, 1H), 4.33 (br s, 2H), 4.21 (br s, 2H), 3.89 (br d, J=12.0 Hz, 1H), 3.67 (br s, 2H), 3.51-3.37 (m, 1H), 2.31 (td, J=7.4, 14.8 Hz, 4H), 1.60 (br s, 7H), 1.26 (s, 32H), 0.88 (t, J=6.8 Hz, 6H).

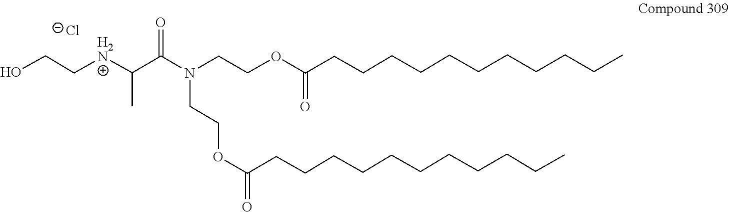

LCMS [M+1]⁺=585.5. ¹H NMR (400 MHz, chloroform-d) δ=5.42-5.30 (m, 1H), 4.77-4.62 (m, 1H), 4.37-4.09 (m, 4H), 3.83 (td, J=5.5, 14.3 Hz, 1H), 3.67 (br s, 2H), 3.42 (td, J=5.6, 14.0 Hz, 1H), 2.41-2.18 (m, 4H), 1.63-1.54 (m, 5H), 1.44 (s, 9H), 1.35-1.20 (m, 34H), 0.89 (t, J=6.8 Hz, 6H).

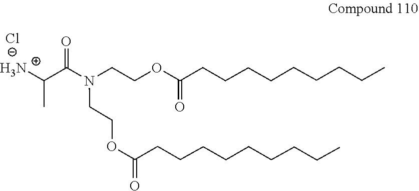 [SMILES: [Cl-] ; CCCCCCCCCC(=O)OCCN(CCOC(=O)CCCCCCCCC)C(=O)C(C)[NH3+]]

LCMS [M+1]⁺=485.4. ¹H NMR (400 MHz, chloroform-d) δ=8.42 (br s, 3H), 4.71-4.58 (m, 1H), 4.39-4.26 (m, 2H), 4.25-4.17 (m, 2H), 3.98-3.82 (m, 1H), 3.67 (br s, 2H), 3.50-3.38 (m, 1H), 2.39-2.24 (m, 4H), 1.63-1.61 (m, 7H), 1.27 (br s, 24H), 0.89 (t, J=6.8 Hz, 6H).

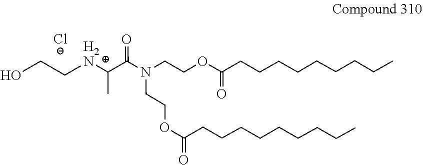

LCMS [M+1]⁺=529.5. ¹H NMR (400 MHz, DMSO-d₆) δ=9.32-8.87 (m, 1H), 8.87-8.44 (m, 1H), 5.21 (br t, J=4.8 Hz, 1H), 4.38 (br d, J=6.0 Hz, 1H), 4.16 (br d, J=6.8 Hz, 4H), 3.83-3.71 (m, 1H), 3.71-3.57 (m, 4H), 3.46 (td, J=5.3, 14.0 Hz, 1H), 3.31-3.21 (m, 1H), 3.00-2.79 (m, 2H), 2.28 (td, J=7.5, 19.2 Hz, 4H), 1.50 (br d, J=6.8 Hz, 4H), 1.37 (br d, J=6.8 Hz, 3H), 1.24 (br s, 24H), 0.85 (br t, J=6.4 Hz, 6H).

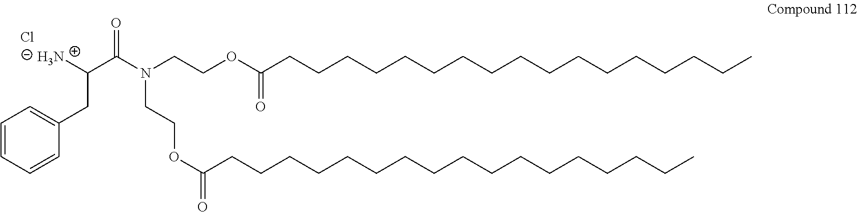 [SMILES: CCCCCCCCCCCCCCCCCC(=O)OCCN(CCOC(=O)CCCCCCCCCCCCCCCCC)C(=O)C(N)Cc1ccccc1 ; [Cl-]]

LCMS [M+1]⁺=785.5. ¹H NMR (400 MHz, chloroform-d) δ=9.06-8.27 (m, 3H), 7.30 (br d, J=6.4 Hz, 4H), 7.27-7.22 (m, 1H), 4.82 (br dd, J=4.4, 8.8 Hz, 1H), 4.24-3.98 (m, 4H), 3.89 (td, J=5.2, 13.6 Hz, 1H), 3.54 (br dd, J=4.0, 13.2 Hz, 1H), 3.32-3.16 (m, 3H), 3.11-3.00 (m, 1H), 2.27 (t, J=7.6 Hz, 4H), 1.60-1.51 (m, 4H), 1.26 (s, 56H), 0.94-0.84 (m, 6H).

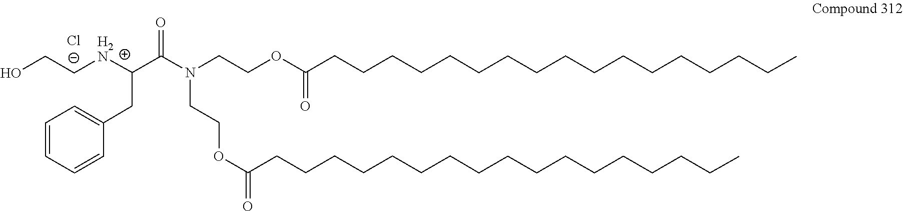

LCMS [M+1]⁺=829.7. ¹H NMR (400 MHz, DMSO-d₆) δ=9.54-9.12 (m, 1H), 9.07-8.72 (m, 1H), 7.51-7.09 (m, 5H), 5.24 (br s, 1H), 4.86-4.51 (m, 1H), 4.14-4.03 (m, 1H), 3.97 (br dd, J=5.2, 10.9 Hz, 1H), 3.82 (br t, J=5.2 Hz, 2H), 3.74-3.59 (m, 3H), 3.45-3.34 (m, 2H), 3.22-3.11 (m, 1H), 3.08-2.84 (m, 4H), 2.23 (br t, J=7.2 Hz, 4H), 1.47 (br d, J=7.2 Hz, 4H), 1.23 (br s, 56H), 0.85 (br t, J=6.8 Hz, 6H).

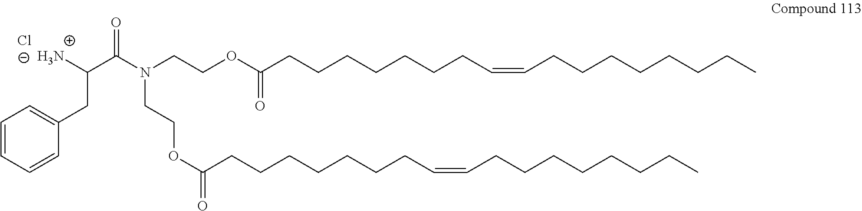 [SMILES: CCCCCCCC/C=C\CCCCCCCC(=O)OCCN(CCOC(=O)CCCCCCC/C=C\CCCCCCCC)C(=O)C([NH3+])Cc1ccccc1 ; [Cl-]]

LCMS [M+1]⁺=781.5. ¹H NMR (400 MHz, chloroform-d) δ=8.71 (br s, 3H), 7.30 (br d, J=12.8 Hz, 4H), 7.27-7.22 (m, 1H), 5.35 (br t, J=6.0 Hz, 4H), 4.86 (br d, J=1.2 Hz, 1H), 4.26-3.97 (m, 4H), 3.95-3.82 (m, 1H), 3.55 (br d, J=11.6 Hz, 1H), 3.34-3.16 (m, 3H), 3.14-2.98 (m, 1H), 2.27 (br t, J=7.6 Hz, 4H), 2.09-1.95 (m, 8H), 1.58 (td, J=6.4, 13.5 Hz, 4H), 1.39-1.19 (m, 40H), 0.88 (t, J=6.8 Hz, 6H).

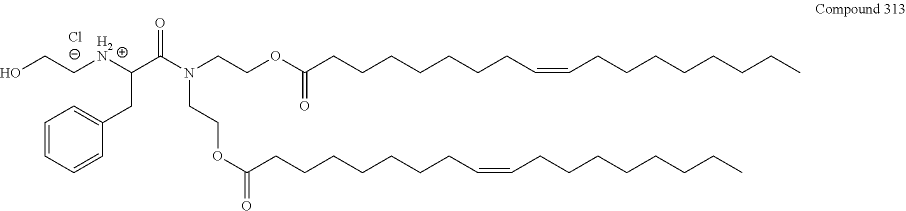

LCMS [M+1]⁺=825.7. ¹H NMR (400 MHz, DMSO-d₆) δ=9.38-9.17 (m, 1H), 9.03-8.83 (m, 1H), 7.35-7.23 (m, 3H), 7.24-7.14 (m, 2H), 5.38-5.28 (m, 4H), 5.25 (br s, 1H), 4.66 (br d, J=1.2 Hz, 1H), 4.12-4.01 (m, 1H), 4.00-3.91 (m, 1H), 3.82 (br t, J=5.6 Hz, 2H), 3.72-3.60 (m, 3H), 3.46-3.36 (m, 2H), 3.20-3.11 (m, 1H), 3.07-2.91 (m, 3H), 2.91 (br d, J=5.2 Hz, 1H), 2.23 (t, J=7.6 Hz, 4H), 2.03-1.91 (m, 8H), 1.54-1.40 (m, 4H), 1.33-1.19 (m, 41H), 0.89-0.79 (m, 6H).

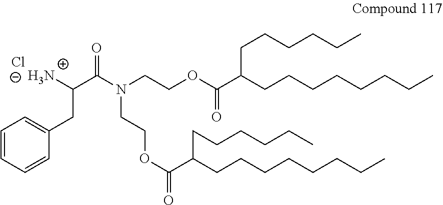 [SMILES: CCCCCCCCC(CCCCCC)C(=O)OCCN(CCOC(=O)C(CCCCCC)CCCCCCCC)C(=O)C([NH3+])Cc1ccccc1 ; [Cl-]]

LCMS [M+1]⁺=729.5. ¹H NMR (400 MHz, chloroform-d) δ=7.29 (d, J=7.6 Hz, 2H), 7.26-7.18 (m, 3H), 4.26-3.91 (m, 5H), 3.78 (td, J=5.6, 14.0 Hz, 1H), 3.51-3.23 (m, 3H), 2.99 (dd, J=7.2, 13.2 Hz, 1H), 2.82 (dd, J=7.2, 13.0 Hz, 1H), 2.37-2.24 (m, 2H), 1.63-1.51 (m, 4H), 1.49-1.39 (m, 4H), 1.25 (br s, 40H), 0.95-0.80 (m, 12H).

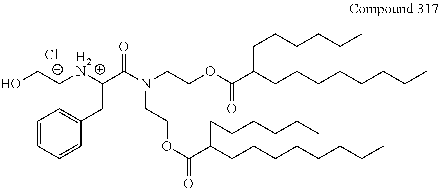

LCMS [M+1]⁺=773.7. ¹H NMR (400 MHz, DMSO-d₆) δ=9.42-9.10 (m, 1H), 9.10-8.78 (m, 1H), 7.36-7.05 (m, 5H), 5.40-5.04 (m, 1H), 4.77-4.49 (m, 1H), 4.11 (td, J=5.2, 10.4 Hz, 1H), 3.95 (td, J=5.6, 11.2 Hz, 1H), 3.91-3.73 (m, 2H), 3.72-3.57 (m, 3H), 3.37 (br d, J=7.2 Hz, 2H), 3.24-3.14 (m, 1H), 3.14-3.04 (m, 1H), 3.07-2.94 (m, 2H), 2.93-2.80 (m, 1H), 2.31-2.18 (m, 2H), 1.55-1.33 (m, 8H), 1.22 (br s, 40H), 0.92-0.77 (m, 12H).

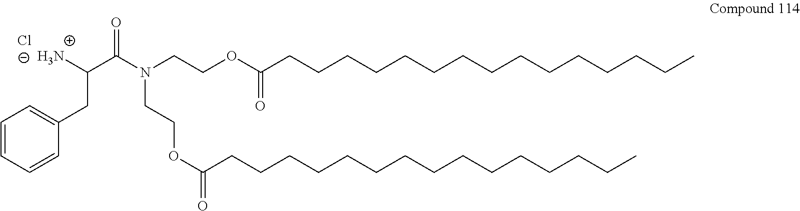 [SMILES: CCCCCCCCCCCCCCCC(=O)OCCN(CCOC(=O)CCCCCCCCCCCCCCC)C(=O)C([NH3+])Cc1ccccc1 ; [Cl-]]

LCMS [M+1]⁺=729.5. ¹H NMR (400 MHz, chloroform-d) δ=8.71 (br s, 3H), 7.30 (br d, J=11.6 Hz, 4H), 7.27-7.22 (m, 1H), 4.86 (br s, 1H), 4.23-3.98 (m, 4H), 3.95-3.83 (m, 1H), 3.55 (br dd, J=3.6, 12.9 Hz, 1H), 3.33-3.17 (m, 3H), 3.12-3.00 (m, 1H), 2.27 (dt, J=2.3, 7.6 Hz, 4H), 1.57 (td, J=6.8, 19.6 Hz, 4H), 1.26 (br s, 48H), 0.89 (t, J=6.8 Hz, 6H).

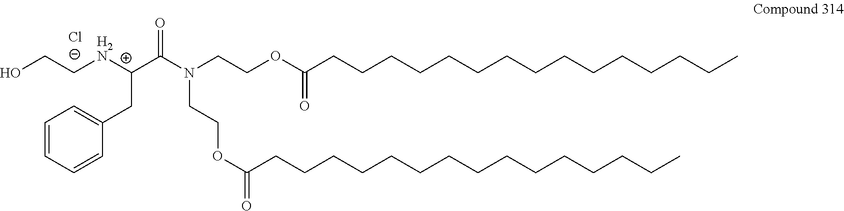

LCMS [M+1]⁺=773.7. ¹H NMR (400 MHz, DMSO-d₆) δ=9.41-9.14 (m, 1H), 9.09-8.76 (m, 1H), 7.36-7.16 (m, 5H), 5.22 (br d, J=4.8 Hz, 1H), 4.71-4.58 (m, 1H), 4.12-4.03 (m, 1H), 4.01-3.91 (m, 1H), 3.82 (br t, J=5.6 Hz, 2H), 3.73-3.58 (m, 3H), 3.40-3.34 (m, 2H), 3.14 (br s, 1H), 3.07-2.83 (m, 4H), 2.23 (t, J=7.6 Hz, 4H), 1.47 (br d, J=6.4 Hz, 4H), 1.23 (s, 48H), 0.85 (t, J=6.8 Hz, 6H).

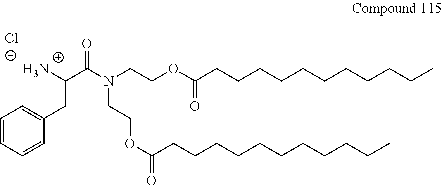 [SMILES: [Cl-] ; CCCCCCCCCCCC(=O)OCCN(CCOC(=O)CCCCCCCCCCC)C(=O)C([NH3+])Cc1ccccc1]

LCMS [M+1]⁺=617.5. ¹H NMR (400 MHz, chloroform-d) δ=7.32-7.27 (m, 2H), 7.26-7.17 (m, 3H), 4.15 (tt, J=6.0, 12.0 Hz, 2H), 4.10-4.02 (m, 2H), 3.99 (br t, J=6.8 Hz, 1H), 3.80 (td, J=5.2, 14.0 Hz, 1H), 3.47-3.20 (m, 3H), 2.99 (dd, J=7.2, 13.2 Hz, 1H), 2.91-2.81 (m, 1H), 2.28 (q, J=7.6 Hz, 4H), 1.58 (br d, J=6.0 Hz, 4H), 1.26 (s, 32H), 0.88 (t, J=6.8 Hz, 6H).

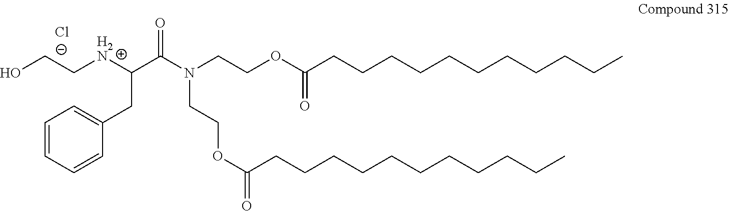

LCMS [M+1]⁺=661.5. ¹H NMR (400 MHz, DMSO-d₆) δ=9.96-9.23 (m, 1H), 9.22-8.59 (m, 1H), 7.45-7.07 (m, 5H), 5.26 (br s, 1H), 4.66 (br d, J=4.0 Hz, 1H), 4.06 (br d, J=5.6 Hz, 1H), 4.01-3.90 (m, 1H), 3.82 (br t, J=5.2 Hz, 2H), 3.75-3.58 (m, 3H), 3.36 (br d, J=13.2 Hz, 2H), 3.23-3.12 (m, 1H), 3.07-2.94 (m, 3H), 2.89 (br d, J=5.6 Hz, 1H), 2.24 (br t, J=7.6 Hz, 4H), 1.47 (br d, J=6.4 Hz, 4H), 1.23 (br s, 32H), 0.85 (br t, J=6.4 Hz, 6H).

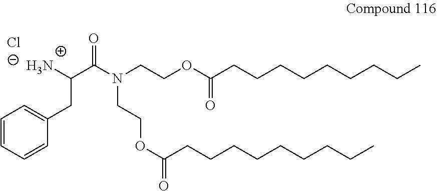 [SMILES: CCCCCCCCCC(=O)OCCN(CCOC(=O)CCCCCCCCC)C(=O)C([NH3+])Cc1ccccc1 ; [Cl-]]

LCMS [M+1]⁺=605.5. ¹H NMR (400 MHz, chloroform-d) δ=7.29 (s, 2H), 7.25-7.17 (m, 3H), 4.24-4.09 (m, 2H), 4.08-3.98 (m, 2H), 3.93 (t, J=7.2 Hz, 1H), 3.80 (td, J=5.3, 14.0 Hz, 1H), 3.48-3.30 (m, 2H), 3.29-3.21 (m, 1H), 2.97 (dd, J=7.6, 13.2 Hz, 1H), 2.82 (dd, J=6.8, 13.2 Hz, 1H), 2.28 (q, J=8.0 Hz, 4H), 1.65-1.52 (m, 4H), 1.38-1.19 (m, 25H), 0.88 (t, J=6.8 Hz, 6H).

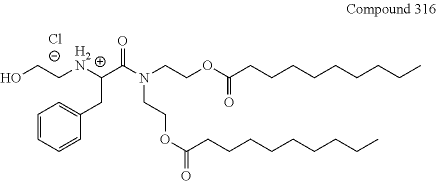

LCMS [M+1]⁺=605.5. ¹H NMR (400 MHz, DMSO-d₆) δ=9.54-9.10 (m, 1H), 9.10-8.79 (m, 1H), 7.48-7.08 (m, 5H), 5.25 (br s, 1H), 4.66 (br d, J=2.4 Hz, 1H), 4.13-3.92 (m, 2H), 3.83 (br t, J=5.2 Hz, 2H), 3.75-3.58 (m, 3H), 3.44-3.36 (m, 2H), 3.15 (br d, J=6.0 Hz, 1H), 3.00 (br d, J=9.6 Hz, 4H), 2.24 (br t, J=7.2 Hz, 4H), 1.48 (br d, J=6.4 Hz, 4H), 1.23 (br s, 24H), 0.85 (br t, J=6.4 Hz, 6H).

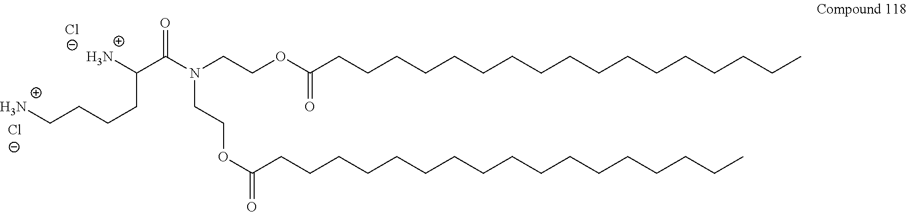 [SMILES: [Cl-] ; CCCCCCCCCCCCCCCCCC(=O)OCCN(CCOC(=O)CCCCCCCCCCCCCCCCC)C(=O)C([NH3+])CCCC[NH3+] ; [Cl-]]

LCMS [M+1]⁺=766.7; [M+1]⁺/2=383.9. ¹H NMR (400 MHz, DMSO-d₆) δ=8.42-8.00 (m, 3H), 7.95-7.64 (m, 3H), 4.31-4.04 (m, 5H), 3.84-3.65 (m, 2H), 3.59-3.49 (m, 1H), 3.47-3.37 (m, 2H), 2.79-2.69 (m, 2H), 2.36-2.20 (m, 5H), 1.74-1.62 (m, 2H), 1.59-1.45 (m, 7H), 1.44-1.33 (m, 3H), 1.23 (s, 58H), 0.85 (br t, J=6.4 Hz, 6H).

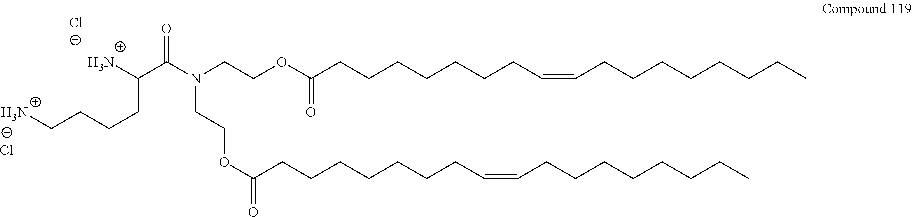 [SMILES: [Cl-] ; [Cl-] ; CCCCCCCC/C=C\CCCCCCCC(=O)OCCN(CCOC(=O)CCCCCCC/C=C\CCCCCCCC)C(=O)C([NH3+])CCCC[NH3+]]

LCMS [M+1]⁺=762.7; [M/2+1]⁺=382.0. ¹H NMR (400 MHz, DMSO-d₆) δ=8.34-7.96 (m, 6H), 5.40-5.22 (m, 4H), 4.25-4.09 (m, 4H), 3.84-3.68 (m, 2H), 3.60-3.41 (m, 2H), 2.78-2.69 (m, 2H), 2.38-2.23 (m, 4H), 2.02-1.94 (m, 8H), 1.74-1.65 (m, 2H), 1.56-1.48 (m, 5H), 1.40 (td, J=7.2, 13.6 Hz, 2H), 1.32-1.22 (m, 42H), 0.88-0.83 (m, 6H).

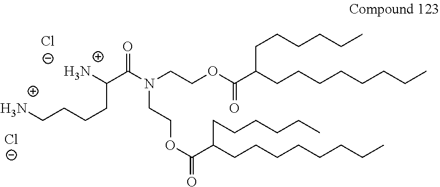 [SMILES: [Cl-] ; CCCCCCCCC(CCCCCC)C(=O)OCCN(CCOC(=O)C(CCCCCC)CCCCCCCC)C(=O)C([NH3+])CCCC[NH3+] ; [Cl-]]

LCMS [M+1]⁺=710.6. ¹H NMR (400 MHz, DMSO-d₆) δ=8.70-7.61 (m, 5H), 4.33-4.00 (m, 5H), 3.84-3.50 (m, 4H), 2.82-2.64 (m, 2H), 2.38-2.23 (m, 2H), 1.78-1.61 (m, 2H), 1.59-1.34 (m, 12H), 1.23 (br s, 40H), 0.85 (br t, J=6.8 Hz, 12H).

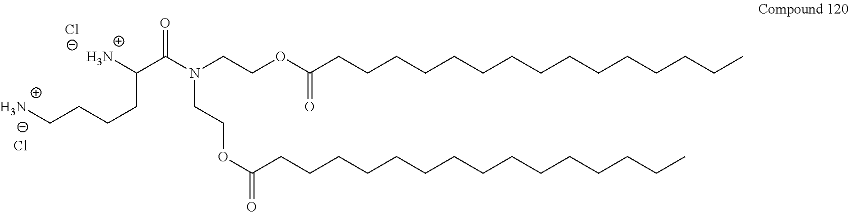 [SMILES: [Cl-] ; CCCCCCCCCCCCCCCC(=O)OCCN(CCOC(=O)CCCCCCCCCCCCCCC)C(=O)C([NH3+])CCCC[NH3+] ; [Cl-]]

LCMS [M+1]⁺=710.5. ¹H NMR (400 MHz, DMSO-d₆) δ=8.44-7.88 (m, 6H), 4.29-4.06 (m, 5H), 3.84-3.68 (m, 2H), 3.60-3.50 (m, 1H), 2.73 (br s, 2H), 2.36-2.23 (m, 4H), 1.70 (br d, J=6.8 Hz, 2H), 1.61-1.46 (m, 6H), 1.43-1.36 (m, 2H), 1.23 (s, 48H), 0.85 (br t, J=6.4 Hz, 6H).

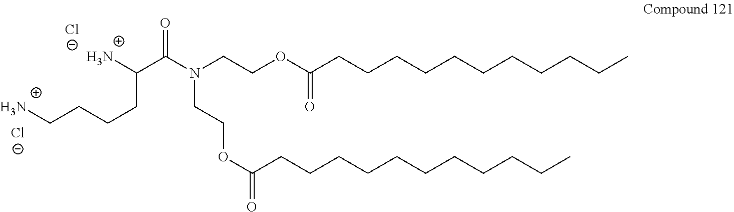 [SMILES: Cl ; CCCCCCCCCCCC(=O)OCCN(CCOC(=O)CCCCCCCCCCC)C(=O)C([NH3+])CCCCN ; [Cl-]]

LCMS [M+1]⁺=598.6. ¹HNMR (400 MHz, chloroform-d) δ=8.38 (s, 3H), 8.06-7.90 (m, 3H), 4.81-4.60 (m, 1H), 4.54-3.99 (m, 4H), 3.93-3.43 (m, 4H), 3.34-3.06 (m, 2H), 2.76 (s, 2H), 2.43-2.20 (m, 4H), 2.09-1.78 (m, 4H), 1.67-1.52 (m, 4H), 1.27 (s, 32H), 0.97-0.68 (m, 6H).

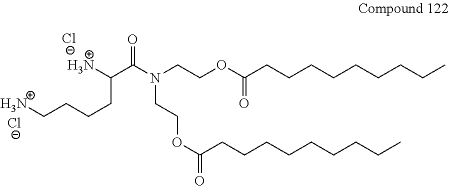 [SMILES: [Cl-] ; [Cl-] ; CCCCCCCCCC(=O)OCCN(CCOC(=O)CCCCCCCCC)C(=O)C([NH3+])CCCC[NH3+]]

LCMS [M+1]⁺=542.5. ¹H NMR (400 MHz, DMSO-d₆) δ=8.46-8.01 (m, 6H), 4.37-4.02 (m, 5H), 3.88-3.67 (m, 2H), 3.61-3.47 (m, 1H), 3.46-3.35 (m, 1H), 2.80-2.66 (m, 2H), 2.41-2.20 (m, 4H), 1.70 (br d, J=6.0 Hz, 2H), 1.62-1.38 (m, 9H), 1.24 (br s, 24H), 0.94-0.76 (m, 6H).

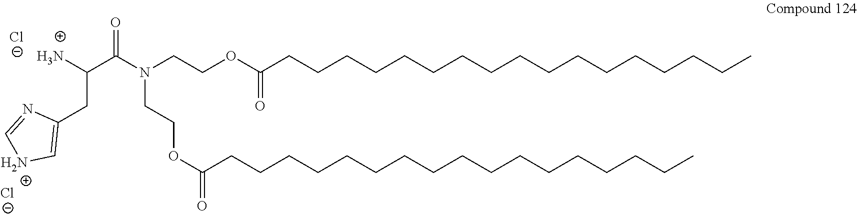 [SMILES: [Cl-] ; CCCCCCCCCCCCCCCCCC(=O)OCCN(CCOC(=O)CCCCCCCCCCCCCCCCC)C(=O)C([NH3+])CC1=C[NH2+]C=N1 ; [Cl-]]

LCMS [M+1]⁺=775.6. ¹H NMR (400 MHz, DMSO-d₆) δ=15.18-13.82 (m, 2H), 9.22-8.91 (m, 1H), 8.34 (br s, 3H), 7.43 (br s, 1H), 4.60 (br d, J=2.4 Hz, 1H), 4.29-4.09 (m, 4H), 3.98-3.87 (m, 1H), 3.82-3.73 (m, 1H), 3.67-3.57 (m, 1H), 3.51-3.42 (m, 2H), 3.24-3.15 (m, 1H), 3.04 (br dd, J=9.6, 15.2 Hz, 1H), 2.34-2.23 (m, 4H), 1.50 (br t, J=6.4 Hz, 5H), 1.24 (s, 56H), 0.86 (t, J=6.8 Hz, 6H).

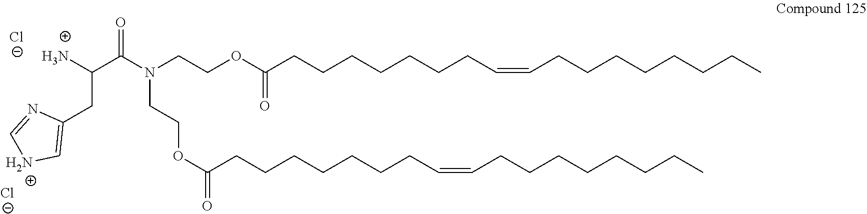 [SMILES: [Cl-] ; CCCCCCCC/C=C\CCCCCCCC(=O)OCCN(CCOC(=O)CCCCCCC/C=C\CCCCCCCC)C(=O)C([NH3+])CC1=C[NH2+]C=N1 ; [Cl-]]

LCMS [M+1]⁺=771.7; [M/2+1]⁺=386.5 ¹H NMR (400 MHz, DMSO-d₆) δ=15.28-13.62 (m, 2H), 9.16-8.81 (m, 1H), 8.39-8.25 (m, 2H), 7.44-7.34 (m, 1H), 5.38-5.24 (m, 4H), 4.63-4.52 (m, 1H), 4.26-4.18 (m, 1H), 4.18-4.06 (m, 3H), 3.96-3.85 (m, 1H), 3.82-3.74 (m, 1H), 3.64-3.54 (m, 1H), 3.50-3.39 (m, 2H), 3.22-3.14 (m, 1H), 3.07-2.97 (m, 1H), 2.32-2.23 (m, 4H), 2.01-1.94 (m, 8H), 1.54-1.45 (m, 4H), 1.24 (br s, 40H), 0.88-0.82 (m, 6H).

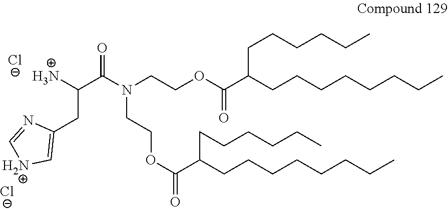 [SMILES: [Cl-] ; CCCCCCCCC(CCCCCC)C(=O)OCCN(CCOC(=O)C(CCCCCC)CCCCCCCC)C(=O)C([NH3+])CC1=C[NH2+]C=N1 ; [Cl-]]

LCMS [M+1]⁺=719.7. ¹H NMR (400 MHz, DMSO-d₆) δ=15.49-14.74 (m, 1H), 14.57-13.86 (m, 1H), 9.42-8.73 (m, 1H), 8.36-8.26 (m, 2H), 7.59-7.25 (m, 1H), 4.67-4.58 (m, 1H), 4.24-4.07 (m, 4H), 4.00-3.91 (m, 1H), 3.79-3.67 (m, 2H), 3.21-3.16 (m, 1H), 3.06-2.99 (m, 1H), 2.33-2.28 (m, 2H), 1.48 (d, J=6.8 Hz, 4H), 1.44-1.38 (m, 4H), 1.25-1.17 (m, 40H), 0.87-0.81 (m, 12H).

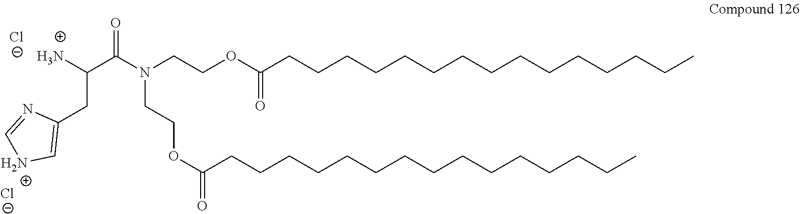 [SMILES: [Cl-] ; CCCCCCCCCCCCCCCC(=O)OCCN(CCOC(=O)CCCCCCCCCCCCCCC)C(=O)C([NH3+])CC1=C[NH2+]C=N1 ; [Cl-]]

LCMS [M+1]⁺=719.6. ¹H NMR (400 MHz, DMSO-d₆) δ=15.39-13.64 (m, 2H), 9.21-8.78 (m, 1H), 8.60-8.03 (m, 3H), 7.59-7.19 (m, 1H), 4.65-4.50 (m, 1H), 4.25-4.08 (m, 4H), 3.93-3.74 (m, 2H), 3.63-3.55 (m, 1H), 3.51-3.41 (m, 2H), 3.21-3.12 (m, 1H), 3.08-2.96 (m, 1H), 2.33-2.25 (m, 4H), 1.54-1.46 (m, 4H), 1.23 (br s, 48H), 0.90-0.80 (m, 6H).

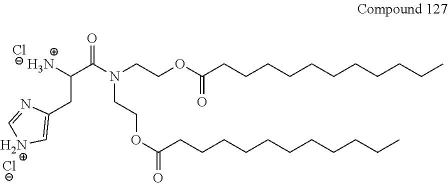 [SMILES: [Cl-] ; CCCCCCCCCCCC(=O)OCCN(CCOC(=O)CCCCCCCCCCC)C(=O)C([NH3+])CC1=C[NH2+]C=N1 ; [Cl-]]

LCMS [M+1]⁺=607.5. ¹H NMR (400 MHz, DMSO-d₆) δ=9.31-8.81 (m, 1H), 8.52-8.10 (m, 3H), 7.62-7.16 (m, 1H), 4.62-4.48 (m, 1H), 4.29-4.05 (m, 4H), 3.97-3.74 (m, 2H), 3.66-3.50 (m, 1H), 3.24-3.11 (m, 1H), 3.07-2.94 (m, 1H), 2.71-2.64 (m, 1H), 2.35-2.21 (m, 5H), 1.55-1.43 (m, 4H), 1.29-1.19 (m, 34H), 0.89-0.81 (m, 6H).

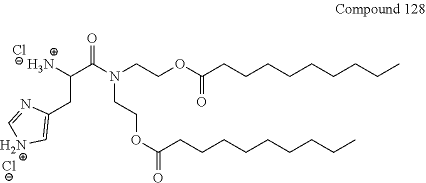 [SMILES: [Cl-] ; CCCCCCCCCC(=O)OCCN(CCOC(=O)CCCCCCCCC)C(=O)C([NH3+])CC1=C[NH2+]C=N1 ; [Cl-]]

LCMS [M+1]⁺=551.5. ¹H NMR (400 MHz, DMSO-d₆) δ=15.33-14.69 (m, 1H), 14.44-13.90 (m, 1H), 9.20-8.86 (m, 1H), 8.38 (s, 3H), 7.44 (s, 1H), 4.67-4.55 (m, 1H), 4.28-4.09 (m, 4H), 4.05-3.91 (m, 1H), 3.85-3.78 (m, 1H), 3.66-3.56 (m, 1H), 3.50-3.40 (m, 2H), 3.26-3.17 (m, 1H), 3.04 (dd, J=9.6, 15.2 Hz, 1H), 2.35-2.22 (m, 4H), 1.50 (t, J=6.8 Hz, 4H), 1.23 (s, 26H), 0.88-0.80 (m, 6H).

### Representative Procedure for Synthesis of Series 100 and 300 Serine Compounds

### Synthesis of Compound 2:

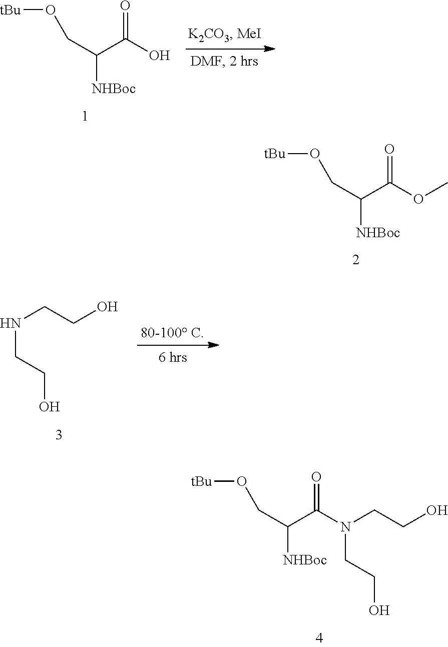 [SMILES: CC(C)(C)OCC(NC(=O)OC(C)(C)C)C(=O)O ; COC(=O)C(COC(C)(C)C)NC(=O)OC(C)(C)C ; OCCNCCO ; CC(C)(C)OCC(NC(=O)OC(C)(C)C)C(=O)N(CCO)CCO]

To a solution of Compound 1 (10 g, 38.26 mmol, 1 eq.) in DMF (70 mL) was added K₂CO₃ (15.86 g, 114.80 mmol, 3 eq.) and CH₃I (5.98 g, 42.10 mmol, 2.62 mL, 1.1 eq.). The mixture was stirred at 20° C. for 2 hours. TLC (Petroleum ether:Ethyl acetate=3:1, R_{f}=0.8) indicated Compound 1 was consumed completely and one new spot formed. The reaction mixture was partitioned between water (70 mL) and ethyl acetate (100 mL). Organic layer was washed with water (50 mL), brine (20 mL), dried over Na₂SO₄ to give Compound 2 (10.4 g, 37.77 mmol, 98.70% yield) as light yellow solid.

Compound 2: ¹H NMR (400 MHz, chloroform-d) δ=5.36 (br d, J=8.4 Hz, 1H), 4.49-4.34 (m, 1H), 3.80 (dd, J=2.8, 9.2 Hz, 1H), 3.74 (s, 31H), 3.56 (dd, J=3.2, 9.2 Hz, 1H), 1.46 (s, 9H), 1.14 (s, 9H).

Synthesis of compound 4:

A mixture of Compound 2 (5 g, 18.16 mmol, 1 eq.) and Compound 3 (3.82 g, 36.32 mmol, 1.75 mL, 2 eq.) was degassed and purged with N₂ for 3 times and then the mixture was heated to 80° C. for 4 hours under N₂ atmosphere. The Compound 2 was remained and then the mixture was heated to 100° C. and stirred at 100° C. for 2 hours. TLC (Dichloromethane:Methanol=15:1, R_{f}=0.45) indicated Compound 2 was consumed and one new spot formed. LC-MS showed desired MS was detected. The reaction mixture was quenched by addition saturated NaHCO₃ solution (10 mL) at 20° C., and then diluted with H₂O (50 mL) and extracted with DCM (50 mL*3). The combined organic layers were washed with brine (50 mL), dried over Na₂SO₄, filtered and concentrated under reduced pressure to give Compound 4 (6 g, 15.50 mmol, 85.34% yield, 90% purity) as colorless gel.

Compound 4: LCMS [M+1]⁺=349.2. ¹H NMR (400 MHz, chloroform-d) δ=5.20 (br d, J=8.0 Hz, 1H), 4.93-4.79 (m, 1H), 3.96-3.59 (m, 9H), 3.52-3.31 (m, 3H), 1.42 (s, 9H), 1.17 (s, 9H).

### Synthesis of Compound 6:

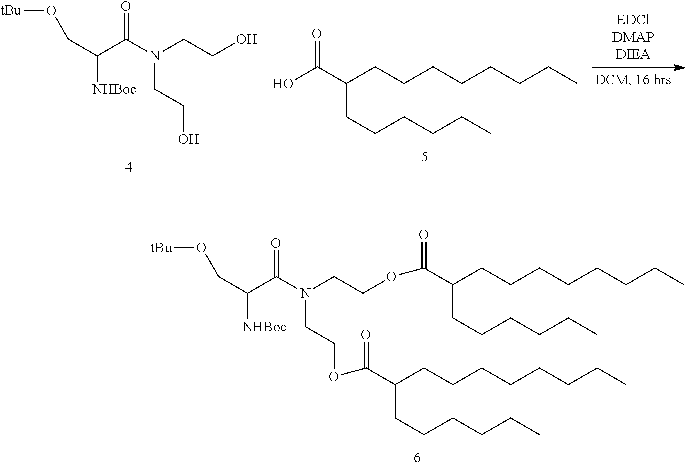 [SMILES: CC(C)(C)OCC(NC(=O)OC(C)(C)C)C(=O)N(CCO)CCO ; CCCCCCCCC(CCCCCC)C(=O)O ; CCCCCCCCC(CCCCCC)C(=O)OCCN(CCOC(=O)C(CCCCCC)CCCCCCCC)C(=O)C(COC(C)(C)C)NC(=O)OC(C)(C)C]

To a solution of Compound 5 (883.12 mg, 3.44 mmol, 2 eq.) in DCM (6 mL) was added DMAP (42.07 mg, 344.40 μmol, 0.2 eq.), EDCI (825.27 mg, 4.30 mmol, 2.5 eq.), DIEA (667.67 mg, 5.17 mmol, 899.82 μL, 3 eq.) and Compound 4 (600 mg, 1.72 mmol, 1 eq.). The mixture was stirred at 20° C. for 16 hours. LC-MS showed desired MS was detected. TLC (Petroleum ether:Ethyl acetate=5:1, R_{f}=0.58) indicated Compound 4 consumed and new spots formed. The reaction mixture was quenched by saturated NaHCO₃ aqueous solution (10 mL) at 20° C., and then diluted with H₂O (10 mL) and extracted with DCM (30 mL*3). The combined organic layers were washed with brine (20 mL), dried over Na₂SO₄, filtered and concentrated under reduced pressure to give a residue. The residue was purified by column chromatography (SiO₂, Petroleum ether:Ethyl acetate=50:1 to 30:1) to give Compound 6 (900 mg, 1.09 mmol, 63.33% yield, 100% purity) as colorless oil.

Compound 6: LCMS [M−100+1]⁺=725.6. ¹H NMR (400 MHz, chloroform-d) δ=5.28 (br d, J=8.4 Hz, 1H), 4.94-4.72 (m, 1H), 4.41-4.28 (m, 1H), 4.26-4.21 (m, 1H), 4.19-4.06 (m, 3H), 3.90-3.82 (m, 1H), 3.62-3.47 (m, 2H), 3.45-3.32 (m, 2H), 2.40-2.27 (m, 2H), 1.64-1.52 (m, 4H), 1.47-1.40 (m, 13H), 1.25 (br s, 40H), 1.13 (s, 9H), 0.88 (t, J=6.8 Hz, 12H).

### Synthesis of Compound 130:

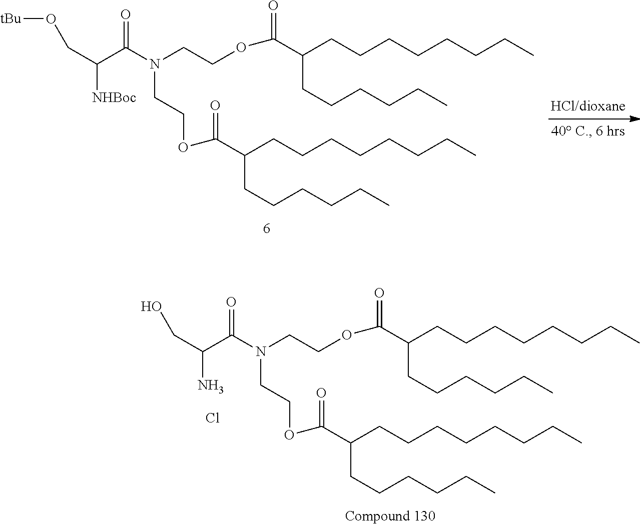 [SMILES: CCCCCCCCC(CCCCCC)C(=O)OCCN(CCOC(=O)C(CCCCCC)CCCCCCCC)C(=O)C(COC(C)(C)C)NC(=O)OC(C)(C)C ; CCCCCCCCC(CCCCCC)C(=O)OCCN(CCOC(=O)C(CCCCCC)CCCCCCCC)C(=O)C(N)CO ; Cl]

A solution of Compound 6 (100 mg, 121.18 μmol, 1 eq.) in HCl/dioxane (4 M, 5.00 mL, 165.05 eq.) was stirred at 40° C. for 6 hours. LC-MS showed Compound 6 was consumed completely and one main peak with desired MS was detected. The reaction mixture was concentrated and lyophilized to give Compound 130 (140 mg, 196.42 μmol, 81.05% yield, 98.98% purity, HCl) as white solid.

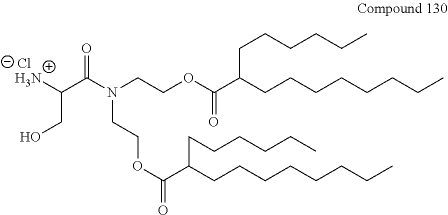 [SMILES: [Cl-] ; CCCCCCCCC(CCCCCC)C(=O)OCCN(CCOC(=O)C(CCCCCC)CCCCCCCC)C(=O)C([NH3+])CO]

LCMS [M+1]⁺=713.6. ¹H NMR (400 MHz, chloroform-d) δ=8.76-7.90 (m, 3H), 5.53-4.97 (m, 1H), 4.73 (br s, 1H), 4.34 (br s, 2H), 4.29-4.03 (m, 3H), 3.96-3.73 (m, 3H), 3.72-3.59 (m, 1H), 3.56-3.43 (m, 1H), 2.33 (td, J=6.0, 11.6 Hz, 2H), 1.57 (br d, J=5.2 Hz, 4H), 1.45 (br s, 4H), 1.26 (s, 40H), 0.88 (t, J=6.8 Hz, 12H).

### Synthesis of Compound 330:

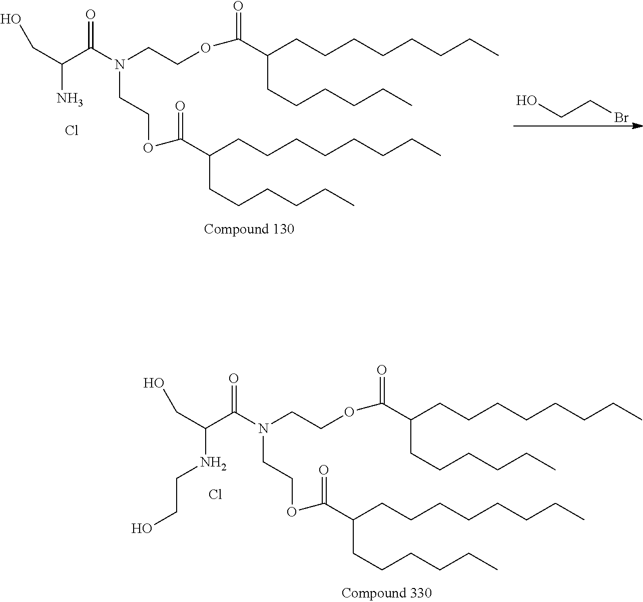

To a solution of Compound 130 (400 mg, 597.88 μmol, 1 eq.) in MeCN (12 mL) was added TEA (181.50 mg, 1.79 mmol, 249.65 μL, 3 eq.) and bromoethanol (373.57 mg, 2.99 mmol, 211.89 μL, 5 eq.). The mixture was stirred at 80° C. for 40 hours. LC-MS showed

Compound 130 was consumed and desired MS was detected. The reaction mixture was concentrated under reduced pressure to give a residue. The residue was purified by prep-TLC (SiO₂, DCM:MeOH=8:1) to give Compound 330 (140 mg, 196.33 μmol, 32.84% yield) as light yellow oil. To a solution of Compound 330 (120 mg, 168.28 μmol, 1 eq.) in dioxane (4.5 mL) was added HCl/dioxane (0.5 M, 673.14 μL, 2 eq.). The mixture was stirred at 20° C. for 2 hours. Special analysis showed desired MS was detected. The reaction mixture was concentrated and lyophilized to give Compound 330 (110 mg, 145.86 μmol, 86.68% yield, 99.39% purity, HCl) as white solid.

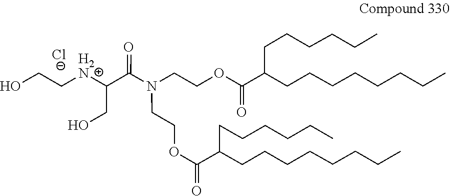

LCMS [M+1]⁺=825.6. ¹H NMR (400 MHz, DMSO-d₆) δ=9.70-8.23 (m, 2H), 5.57 (br s, 1H), 5.22 (br t, J=4.4 Hz, 1H), 4.49 (br s, 1H), 4.21 (br s, 2H), 4.16-4.03 (m, 2H), 3.78 (br d, J=3.2 Hz, 3H), 3.73-3.58 (m, 4H), 3.52 (br d, J=7.2 Hz, 1H), 3.09-2.85 (m, 2H), 2.38-2.21 (m, 2H), 1.55-1.45 (m, 4H), 1.41 (br s, 4H), 1.22 (br s, 40H), 0.85 (br t, J=6.4 Hz, 12H).

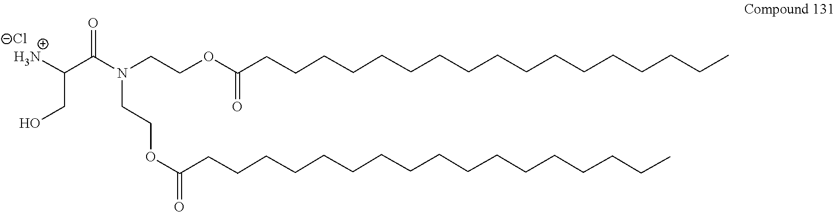 [SMILES: [Cl-] ; CCCCCCCCCCCCCCCCCC(=O)OCCN(CCOC(=O)CCCCCCCCCCCCCCCCC)C(=O)C([NH3+])CO]

LCMS [M+1]⁺=725.6. ¹H NMR (400 MHz, chloroform-d) δ=8.23 (br s, 3H), 5.18 (br d, J=5.2 Hz, 1H), 4.78 (br s, 1H), 4.33 (br s, 2H), 4.20 (br s, 2H), 4.15-4.05 (m, 1H), 3.98-3.75 (m, 3H), 3.73-3.60 (m, 1H), 3.56-3.40 (m, 1H), 2.44-2.21 (m, 4H), 1.59 (br s, 4H), 1.26 (s, 57H), 0.99-0.78 (m, 6H).

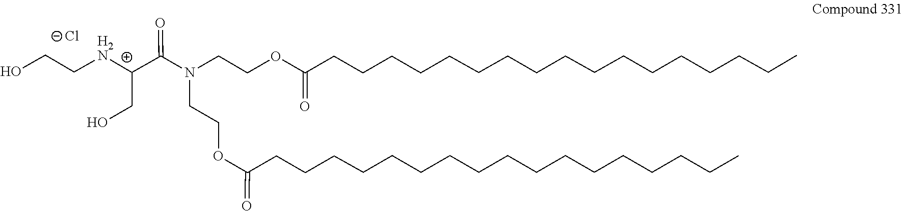

LCMS [M+1]⁺=769.7. ¹H NMR (400 MHz, DMSO-d₆) δ=9.02-8.78 (m, 1H), 8.76-8.51 (m, 1H), 5.58-5.39 (m, 1H), 5.23-5.07 (m, 1H), 4.51-4.36 (m, 1H), 4.16 (br dd, J=4.0, 16.0 Hz, 4H), 3.82-3.49 (m, 8H), 3.03-2.86 (m, 2H), 2.32-2.20 (m, 4H), 1.50 (br dd, J=1.6, 4.0 Hz, 4H), 1.23 (br s, 56H), 0.85 (br s, 6H).

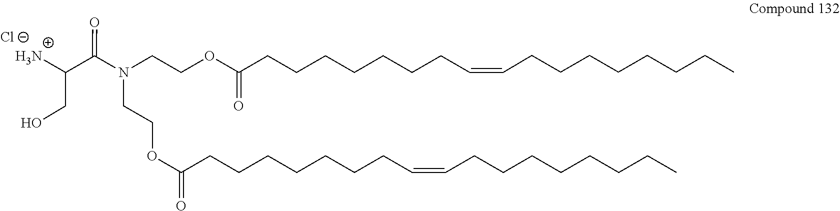 [SMILES: [Cl-] ; CCCCCCCC/C=C\CCCCCCCC(=O)OCCN(CCOC(=O)CCCCCCC/C=C\CCCCCCCC)C(=O)C([NH3+])CO]

LCMS [M+1]*=721.6. ¹H NMR (400 MHz, methanol-d₄) δ=5.35 (t, J=4.4 Hz, 4H), 4.28 (br d, J=4.4 Hz, 2H), 4.23 (t, J=5.6 Hz, 2H), 3.96 (s, 1H), 3.92-3.84 (m, 1H), 3.76 (td, J=5.6, 14.2 Hz, 1H), 3.66 (br dd, J=6.4, 10.7 Hz, 2H), 3.63-3.52 (m, 2H), 2.35 (td, J=7.6, 18.0 Hz, 4H), 2.04 (br d, J=5.6 Hz, 8H), 1.61 (br d, J=3.2 Hz, 4H), 1.31 (br d, J=12.0 Hz, 40H), 0.95-0.86 (m, 6H).

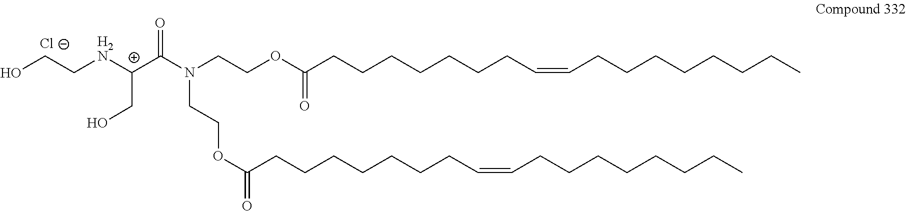

LCMS [M+1]⁺=765.7. ¹H NMR (400 MHz, DMSO-d₆) δ=9.13-8.85 (m, 1H), 8.84-8.52 (m, 1H), 5.54 (br t, J=4.8 Hz, 1H), 5.42-5.24 (m, 4H), 5.19 (br s, 1H), 4.55-4.39 (m, 1H), 4.27-4.04 (m, 4H), 3.84-3.48 (m, 8H), 3.09-2.86 (m, 2H), 2.28 (td, J=7.2, 19.9 Hz, 4H), 1.98 (br d, J=5.6 Hz, 8H), 1.50 (br d, J=4.0 Hz, 4H), 1.24 (br s, 41H), 0.85 (br t, J=6.8 Hz, 6H).

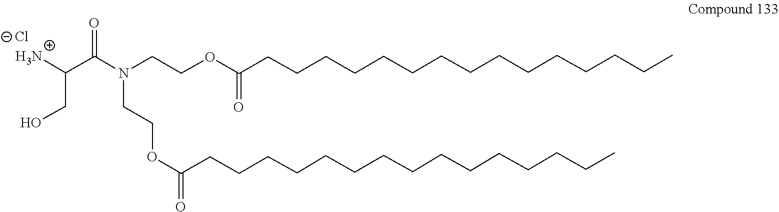 [SMILES: [Cl-] ; CCCCCCCCCCCCCCCC(=O)OCCN(CCOC(=O)CCCCCCCCCCCCCCC)C(=O)C([NH3+])CO]

LCMS [M+1]⁺=669.6. ¹H NMR (400 MHz, chloroform-d) δ=8.47-8.05 (m, 3H), 5.41-5.01 (m, 1H), 4.76 (br s, 1H), 4.33 (br s, 2H), 4.28-4.04 (m, 3H), 3.95-3.76 (m, 3H), 3.73-3.59 (m, 1H), 3.55-3.40 (m, 1H), 2.41-2.20 (m, 4H), 1.59-1.54 (m, 4H), 1.26 (s, 48H), 0.89 (t, J=6.8 Hz, 6H).

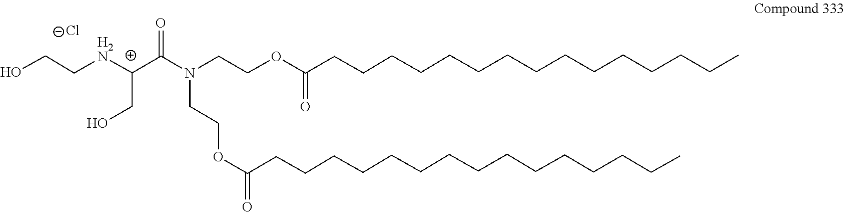

LCMS [M+1]⁺=713.6. ¹H NMR (400 MHz, DMSO-d₆) δ=9.29-8.84 (m, 1H), 8.80-8.40 (m, 1H), 5.60-5.48 (m, 1H), 5.18 (br t, J=5.2 Hz, 1H), 4.46 (br s, 1H), 4.16 (qd, J=5.2, 16.0 Hz, 4H), 3.76 (br dd, J=5.2, 12.8 Hz, 3H), 3.70-3.46 (m, 5H), 3.09-2.86 (m, 2H), 2.36-2.22 (m, 4H), 1.50 (br d, J=5.6 Hz, 4H), 1.23 (s, 48H), 0.92-0.81 (m, 6H).

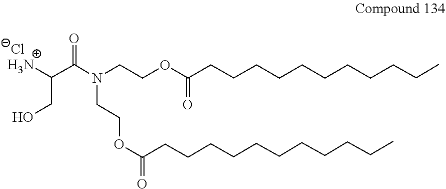 [SMILES: [Cl-] ; CCCCCCCCCCCC(=O)OCCN(CCOC(=O)CCCCCCCCCCC)C(=O)C([NH3+])CO]

LCMS [M+1]⁺=557.5. ¹H NMR (400 MHz, chloroform-d) δ=4.36-4.14 (m, 4H), 3.83 (br d, J=5.2 Hz, 1H), 3.81-3.73 (m, 2H), 3.72-3.63 (m, 2H), 3.61-3.53 (m, 1H), 3.53-3.44 (m, 1H), 2.31 (q, J=7.2 Hz, 4H), 1.64-1.58 (m, 4H), 1.27 (s, 34H), 0.89 (br t, J=6.8 Hz, 6H).

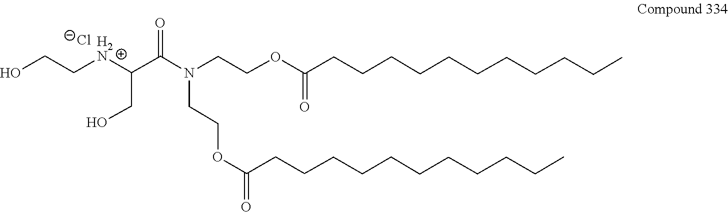

LCMS [M+1]⁺=601.5. ¹H NMR (400 MHz, DMSO-d₆) δ=9.06-8.83 (m, 1H), 8.81-8.56 (m, 1H), 5.53 (br t, J=5.2 Hz, 1H), 5.18 (br t, J=4.8 Hz, 1H), 4.44 (br d, J=4.8 Hz, 1H), 4.24-4.06 (m, 4H), 3.83-3.70 (m, 3H), 3.66 (br d, J=5.2 Hz, 5H), 3.10-2.84 (m, 2H), 2.36-2.22 (m, 4H), 1.50 (br d, J=5.6 Hz, 4H), 1.23 (s, 32H), 0.90-0.79 (m, 6H).

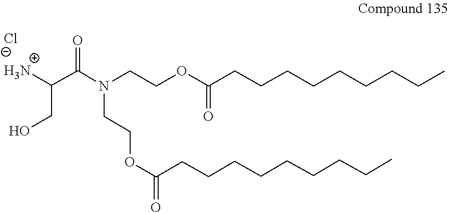 [SMILES: [Cl-] ; CCCCCCCCCC(=O)OCCN(CCOC(=O)CCCCCCCCC)C(=O)C([NH3+])CO]

LCMS [M+1]⁺=501.4. ¹H NMR (400 MHz, chloroform-d) δ=4.49 (br s, 1H), 4.30 (br s, 2H), 4.25-4.14 (m, 2H), 3.98 (br d, J=10.0 Hz, 1H), 3.89-3.74 (m, 3H), 3.72-3.59 (m, 1H), 3.56-3.41 (m, 1H), 2.38-2.24 (m, 4H), 1.59 (br d, J=6.0 Hz, 4H), 1.27 (br s, 24H), 0.89 (t, J=6.8 Hz, 6H).

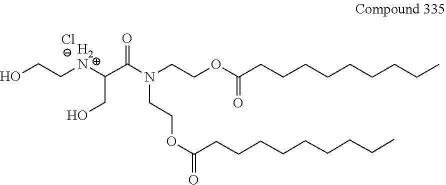

LCMS [M+1]⁺=545.4. ¹H NMR (400 MHz, DMSO-d₆) δ=9.87-7.91 (m, 2H), 5.52 (br s, 1H), 5.16 (br s, 1H), 4.59-4.36 (m, 1H), 4.34-3.94 (m, 4H), 3.92-3.71 (m, 3H), 3.70-3.43 (m, 5H), 3.10-2.96 (m, 1H), 2.93-2.79 (m, 1H), 2.39-2.13 (m, 4H), 1.50 (br s, 4H), 1.24 (br s, 24H), 0.98-0.68 (m, 6H).

### Synthesis of Series 300-Lysine Compounds:

### Synthesis of Compound 3:

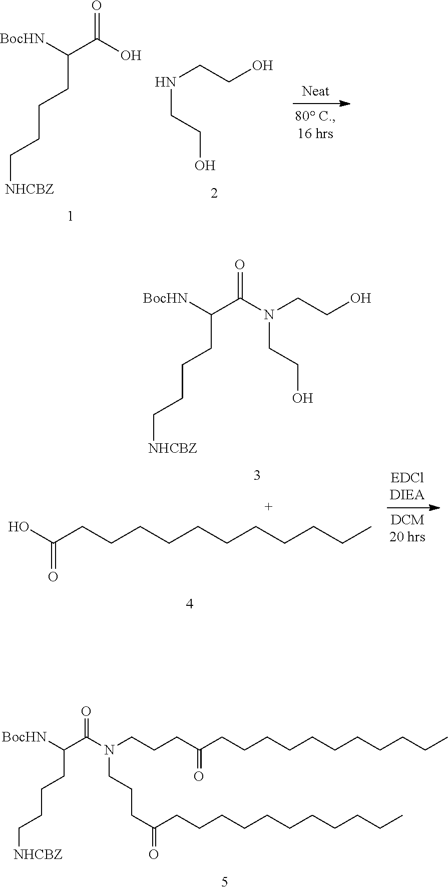

A mixture of Compound 1 (15 g, 38.03 mmol, 1 eq.) and Compound 2 (4.40 g, 41.83 mmol, 4.04 mL, 1.1 eq.) was degassed and purged with N₂ for 3 times, and then the mixture was stirred at 80° C. for 16 hours under N₂ atmosphere. LCMS showed Compound 1 was consumed completely and one main peak with desired MS was detected. The mixture was concentrated to give a residue. The residue was purified by column chromatography (SiO₂, Petroleum ether:DCM:Ethyl acetate=1:1:1 to 0:1:1) to give Compound 3 (8 g, 16.94 mmol, 44.55% yield, 99% purity) as white solid.

Compound 3: LCMS [M+1]⁺=468.4; [M−100]⁺=368.4. ¹H NMR (400 MHz, DMSO-d₆) δ=7.38-7.29 (m, 5H), 7.26-7.19 (m, 1H), 6.80 (d, J=7.6 Hz, 1H), 5.05-4.99 (m, 2H), 4.85-4.77 (m, 1H), 4.68-4.60 (m, 1H), 4.39-4.26 (m, 1H), 3.58-3.33 (m, 8H), 3.27-3.16 (m, 1H), 3.00-2.93 (m, 2H), 1.53-1.46 (m, 2H), 1.38-1.35 (m, 9H), 1.31-1.20 (m, 2H)

### Synthesis of Compound 5:

A mixture of Compound 3 (1 g, 2.14 mmol, 1 eq.), Compound 4 (856.87 mg, 4.28 mmol, 2 eq.), EDCI (1.23 g, 6.42 mmol, 3 eq.), DIEA (829.27 mg, 6.42 mmol, 1.12 mL, 3 eq.), DMAP (130.65 mg, 1.07 mmol, 0.5 eq.) in DCM (10 mL) was degassed and purged with N₂ for 3 times, and then the mixture was stirred at 20° C. for 16 hours under N₂ atmosphere. LCMS showed Compound 3 was consumed completely and one main peak with desired MS was detected. The reaction mixture was concentrated under reduced pressure to give a residue. The residue was diluted with DCM (30 mL) and washed with sat.NH₄Cl (9 mL), dried over Na₂SO₄, filtered and concentrated under reduced pressure to give a residue. The residue was purified by column chromatography (SiO₂, Petroleum ether:Ethyl acetate=30:1 to 5:1) to give Compound 5 (1.13 g, 1.36 mmol, 63.49% yield) as white solid.

Compound 5: LCMS [M+1]⁺=732.5. ¹H NMR (400 MHz, DMSO-d₆) δ=7.39-7.26 (m, 5H), 7.25-7.19 (m, 1H), 6.92-6.83 (m, 1H), 5.07-4.97 (m, 2H), 4.37-4.27 (m, 1H), 4.26-4.17 (m, 1H), 4.17-4.02 (m, 3H), 3.73-3.61 (m, 3H), 3.39-3.33 (m, 1H), 3.01-2.92 (m, 2H), 2.32-2.20 (m, 4H), 1.53-1.42 (m, 6H), 1.41-1.31 (m, 12H), 1.22 (s, 34H), 0.88-0.81 (m, 6H).

### Synthesis of Compound 6:

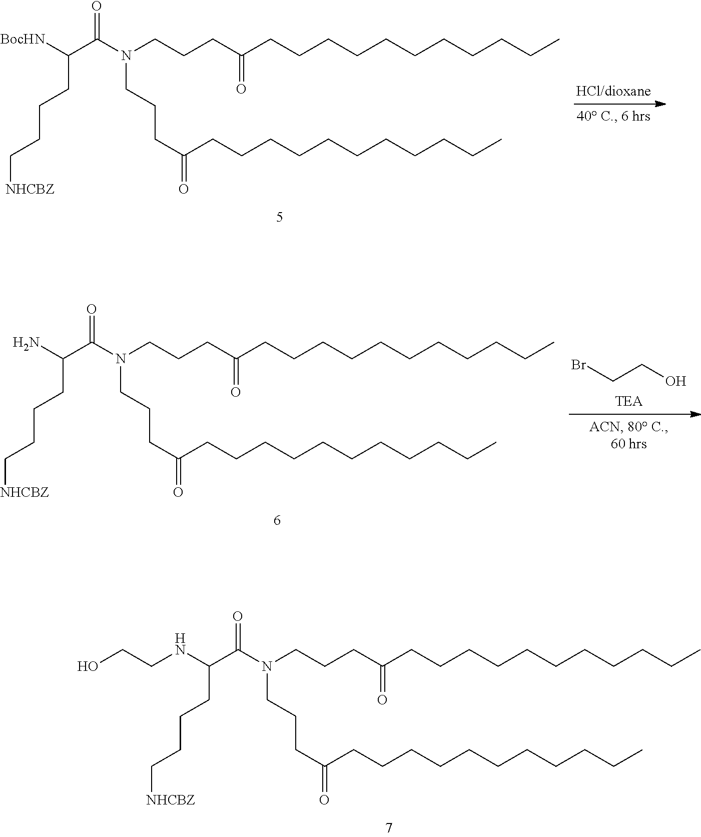

A solution of Compound 5 (1.6 g, 1.92 mmol, 1 eq.) in HCl/Dioxane (40 mL) was stirred at 20° C. for 2 hours. LCMS showed Compound 5 was consumed and desired MS was detected. The reaction was concentrated and lyophilized to give Compound 6 (1.3 g, 1.74 mmol, 90.48% yield, 97.96% purity) as white solid.
Compound 6: LCMS [M+1]⁺=732.5. ¹H NMR (400 MHz, DMSO-d₆) δ=8.34-8.15 (m, 3H), 7.46-7.20 (m, 5H), 5.09-4.94 (m, 2H), 4.32-4.03 (m, 5H), 3.92-3.78 (m, 1H), 3.70 (d, J=15.6 Hz, 1H), 3.59-3.46 (m, 1H), 3.03-2.92 (m, 2H), 2.37-2.20 (m, 4H), 1.76-1.61 (m, 2H), 1.49 (d, J=4.4 Hz, 5H), 1.42-1.14 (m, 36H), 0.93-0.76 (m, 6H).

### Synthesis of Compound 7:

A mixture of Compound 6 (500 mg, 683.02 μmol, 1 eq.), 2-bromoethanol (256.06 mg, 2.05 mmol, 145.24 μL, 3 eq.), TEA (276.46 mg, 2.73 mmol, 380.27 μL, 4 eq.) in DMF (5 mL) was degassed and purged with N₂ for 3 times, and then the mixture was stirred at 80° C. for 16 hours under N₂ atmosphere. LCMS showed Compound 6 was consumed completely and one main peak with desired mass was detected. The reaction mixture was concentrated under reduced pressure to remove solvent. The residue was diluted with DCM (30 mL). The combined organic layers were washed with NH₄Cl (9 mL), dried over Na₂SO₄, filtered and concentrated under reduced pressure to give a residue. The residue was purified by prep-TLC (SiO₂, DCM:MeOH=10:1) to give Compound 7 (180 mg, 231.93 μmol, 33.96% yield) as yellow liquid.

Compound 7: LCMS [M+1]⁺=776.5. ¹H NMR (400 MHz, DMSO-d₆) δ=7.39-7.27 (m, 4H), 7.24-7.18 (m, 1H), 5.05-4.96 (m, 2H), 4.44-4.38 (m, 1H), 4.20-4.01 (m, 4H), 3.70-3.61 (m, 2H), 3.60-3.51 (m, 1H), 3.49-3.43 (m, 2H), 3.42-3.35 (m, 2H), 3.00-2.92 (m, 2H), 2.38-2.30 (m, 1H), 2.30-2.20 (m, 4H), 1.54-1.44 (m, 4H), 1.44-1.32 (m, 5H), 1.23 (s, 34H), 0.85 (t, J=6.6 Hz, 6H).

### Synthesis of Compound 321:

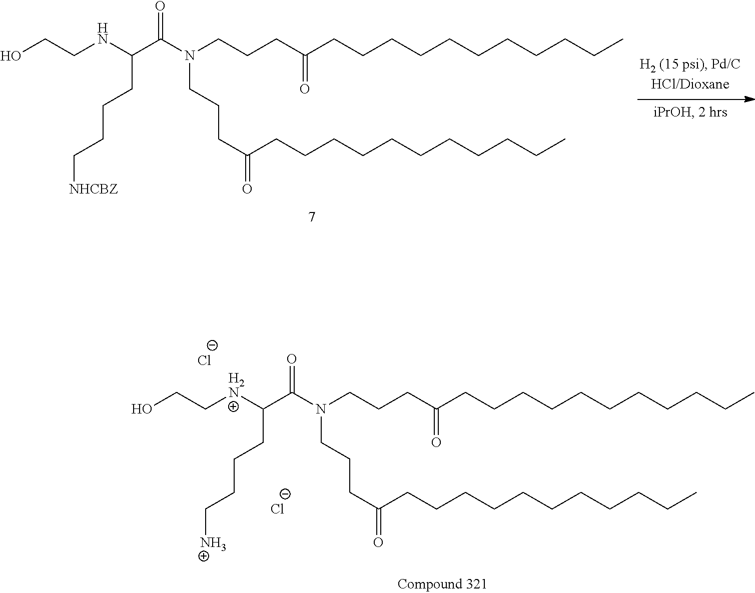

To a solution of Compound 7 (180 mg, 231.93 μmol, 1 eq.) in IPA (20 mL) was added Pd/C (360.00 mg, 338.28 μmol, 10% purity, 1.46 eq.) and HCl/Dioxane (0.5 M, 1.02 mL, 2.2 eq.). The mixture was degassed and purged with H₂ for 3 times, and then the mixture was stirred at 20° C. for 2.5 hours under H₂ atmosphere. TLC (Dichloromethane:Methanol=10:1, P:R_{f}=0.5) indicated Compound 7 was consumed and one new spot formed. The reaction mixture was filtered, and the filtrate was concentrated and lyophilized to give Compound 321 (110 mg, 171.35 μmol, 73.88% yield, HCl) as white solid.

Compound 321: LCMS [M+1]⁺=642.5; [M+1]⁺/2=321.8. ¹H NMR (400 MHz, DMSO-d₆) δ=9.93-9.13 (m, 1H), 9.04-8.39 (m, 1H), 8.08 (s, 3H), 5.23 (s, 1H), 4.44 (s, 1H), 4.26-4.02 (m, 4H), 3.86-3.63 (m, 4H), 3.63-3.43 (m, 2H), 3.01-2.90 (m, 1H), 2.90-2.80 (m, 1H), 2.73 (s, 2H), 2.28 (td, J=7.6, 19.2 Hz, 4H), 1.84 (br d, J=5.6 Hz, 2H), 1.64-1.45 (m, 6H), 1.39 (m, 2H), 1.24 (s, 32H), 0.85 (t, J=6.4 Hz, 6H).

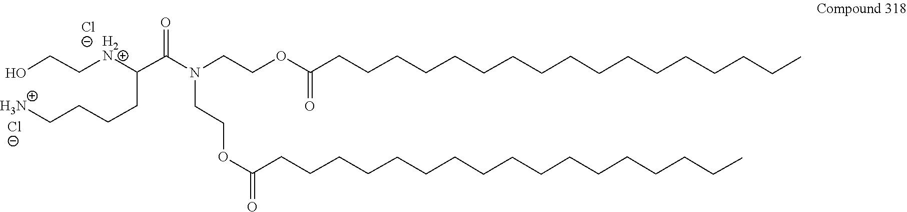

LCMS [M+1]⁺=810.7. ¹H NMR (400 MHz, DMSO-d₆) δ=9.38-9.16 (m, 1H), 8.80-8.64 (m, 1H), 8.00-7.84 (m, 2H), 5.25-5.17 (m, 1H), 4.49-4.38 (m, 1H), 4.20-4.11 (m, 3H), 3.75 (br s, 3H), 3.60-3.45 (m, 2H), 3.39-3.27 (m, 15H), 3.05-2.81 (m, 2H), 2.76-2.68 (m, 1H), 2.32-2.20 (m, 3H), 1.87-1.79 (m, 1H), 1.60-1.47 (m, 5H), 1.40-1.32 (m, 2H), 1.27-1.17 (m, 48H), 0.89-0.80 (m, 6H).

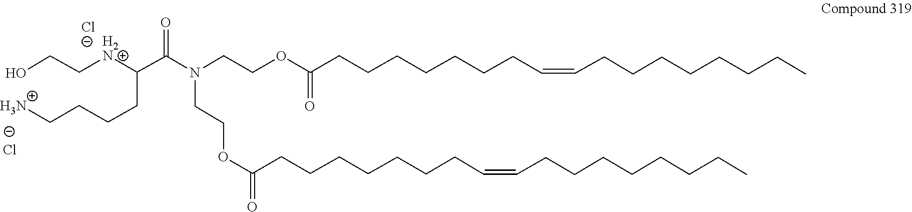

LCMS [M+1]⁺=806.8. ¹H NMR (400 MHz, DMSO-d₆) δ=9.52-9.18 (m, 1H), 8.80-8.62 (m, 1H), 8.06-7.86 (m, 3H), 5.39-5.27 (m, 4H), 5.25-5.19 (m, 1H), 4.51-4.40 (m, 1H), 4.22-4.10 (m, 4H), 3.80-3.64 (m, 4H), 3.60-3.48 (m, 2H), 3.00-2.80 (m, 2H), 2.79-2.69 (m, 2H), 2.35-2.23 (m, 4H), 2.02-1.92 (m, 8H), 1.90-1.76 (m, 2H), 1.58-1.47 (m, 6H), 1.42-1.34 (m, 2H), 1.31-1.22 (m, 40H), 0.91-0.77 (m, 6H).

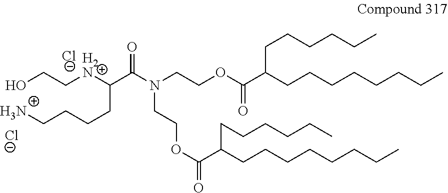

LCMS [M+1]⁺=754.7. ¹H NMR (400 MHz, DMSO-d₆) δ=9.77-9.12 (m, 1H), 9.04-8.46 (m, 1H), 8.27-7.62 (m, 3H), 5.22 (m, 1H), 4.63-4.29 (m, 1H), 4.28-4.03 (m, 4H), 3.86-3.43 (m, 6H), 3.06-2.80 (m, 2H), 2.74 (s, 2H), 2.37-2.36 (m, 1H), 2.37-2.22 (m, 1H), 1.80 (s, 2H), 1.60-1.33 (m, 12H), 1.23 (s, 40H), 0.85 (t, J=6.4 Hz, 12H).

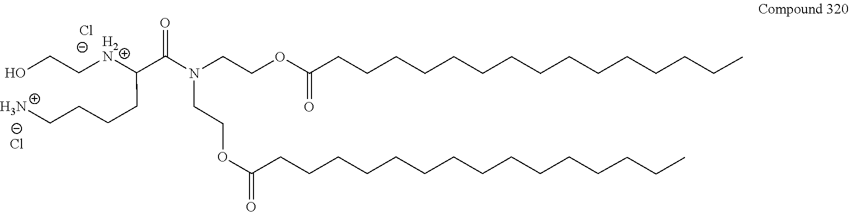

LCMS [M+1]⁺=754.7. ¹H NMR (400 MHz, DMSO-d₆) δ=9.56-9.17 (m, 1H), 8.90-8.43 (m, 1H), 8.22-7.56 (m, 3H), 5.34-5.03 (m, 1H), 4.53-4.29 (m, 1H), 4.26-4.06 (m, 4H), 3.85-3.62 (m, 4H), 3.62-3.46 (m, 2H), 3.01-2.79 (m, 2H), 2.74 (br s, 2H), 2.28 (td, J=7.6, 18.4 Hz, 4H), 1.87-1.73 (m, 2H), 1.59-1.46 (m, 6H), 1.41-1.34 (m, 3H), 1.23 (s, 48H), 0.85 (br t, J=6.4 Hz, 6H).

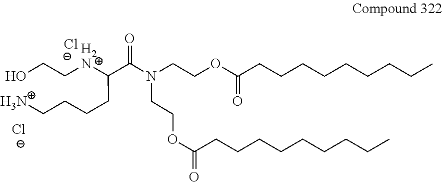

LCMS [M+1]⁺=586.5, LCMS [(M+1)/2]⁺=293.8. ¹H NMR (400 MHz, DMSO-d₆) δ=9.72-9.07 (m, 1H), 8.96-8.41 (m, 1H), 7.96 (br s, 3H), 5.20 (br s, 1H), 4.42 (br s, 1H), 4.29-4.06 (m, 4H), 3.84-3.61 (m, 4H), 3.61-3.44 (m, 2H), 2.92 (br s, 1H), 2.86 (br s, 1H), 2.80-2.64 (m, 2H), 2.28 (td, J=7.4, 19.2 Hz, 4H), 1.91-1.72 (m, 2H), 1.60-1.44 (m, 6H), 1.43-1.33 (m, 2H), 1.32-1.17 (m, 24H), 0.95-0.76 (m, 6H).

### Synthesis of Series 300-Histidine Compounds:

### Synthesis of Compound 2:

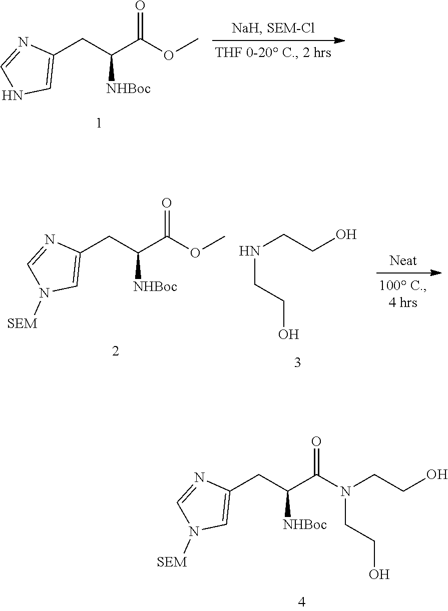

To a solution of Compound 1 (10 g, 37.13 mmol, 1 eq.) in THF (100 mL) was added NaH (1.49 g, 37.13 mmol, 60% purity, 1 eq.) in portions and added dropwise SEM-C₁ (6.19 g, 37.13 mmol, 6.57 mL, 1 eq.) at 0° C. to the mixture. The mixture was stirred at 20° C. for 2 hours. LCMS showed Compound 1 was consumed completely and desired MS was detected. The reaction mixture was added into Sat. NH₄Cl (20 mL) slowly at 0° C., and extracted with Ethyl acetate (50 mL*3). The combined organic layers were dried over Na₂SO₄, filtered and concentrated under reduced pressure to give a residue. The residue was purified by column chromatography (SiO₂, DCM:MeOH=97:3 to 95:5) to give Compound 2 (15.7 g, 32.61 mmol, 87.83% yield, 83% purity) as white solid.

Compound 2: LCMS [M+1]⁺=400.2. ¹H NMR (400 MHz, chloroform-d) δ=7.57-7.44 (m, 1H), 6.92-6.74 (m, 1H), 5.87 (br d, J=8.0 Hz, 1H), 5.39-5.26 (m, 1H), 5.25 (s, 1H), 5.19 (s, 1H), 4.62-4.50 (m, 1H), 3.73 (s, 1H), 3.70 (s, 2H), 3.54-3.37 (m, 2H), 3.19-3.00 (m, 2H), 1.43 (d, J=7.2 Hz, 9H), 0.97-0.85 (m, 2H), −0.01 (s, 9H).

Synthesis of Compound 4: A solution of Compound 3 (4.05 g, 38.54 mmol, 3.72 mL, 2 eq.) and Compound 2 (7.7 g, 19.27 mmol, 1 eq.) was stirred at 100° C. for 4 hours. TLC (Dichloromethane: Methanol=10:1, R_{f}=0.5) indicated Compound 2 was consumed completely and one new spot formed. The residue was diluted with sat.NaHCO₃ (50 mL) and extracted with Ethyl acetate (30 mL*3). The combined organic layers were dried over Na₂SO₄, filtered and concentrated under reduced pressure to give a residue. The residue was purified by column chromatography (SiO₂, DCM:MeOH=96:4 to 93:7) to give Compound 4 (9.8 g, 19.49 mmol, 50.57% yield, 94% purity) as white solid.

Compound 4: LCMS [M+1]⁺=473.3. ¹H NMR (400 MHz, chloroform-d) δ=7.48 (d, J=0.8 Hz, 1H), 6.86 (s, 1H), 5.53 (br d, J=7.6 Hz, 1H), 5.26-5.15 (m, 2H), 4.95-4.81 (m, 1H), 4.18-4.00 (m, 1H), 3.91-3.71 (m, 4H), 3.61 (td, J=3.6, 12 Hz, 1H), 3.46 (t, J=8.0 Hz, 2H), 3.42-3.31 (m, 1H), 3.19 (m, 1H), 3.15-2.99 (m, 2H), 1.44 (s, 9H), 0.97-0.84 (m, 2H), 0.00 (s, 9H).

### Synthesis of Compound 6:

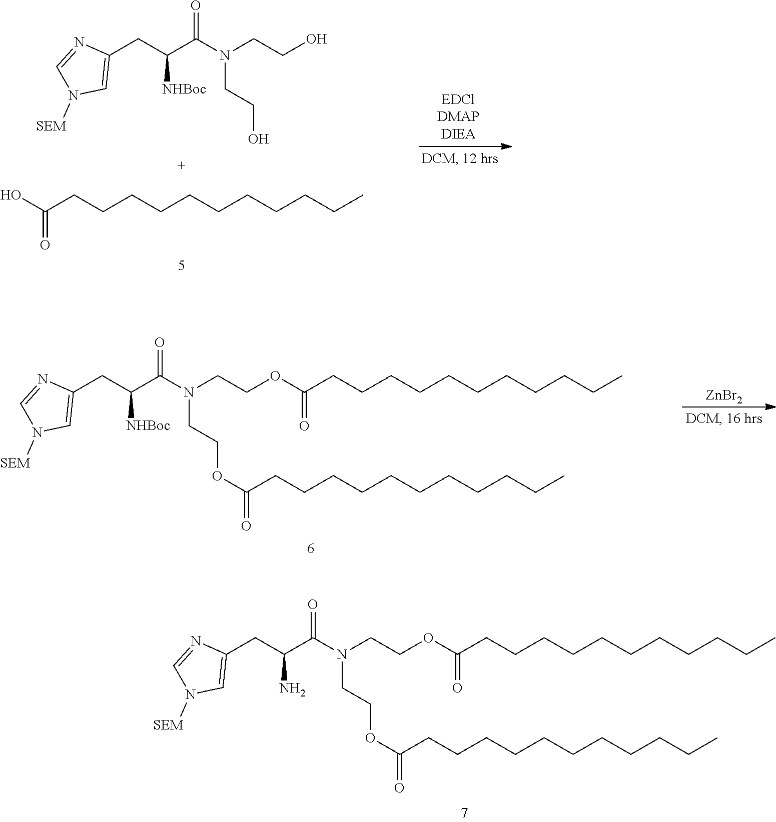 [SMILES: CC(C)(C)OC(=O)N[C@@H](Cc1cn(COCC[Si](C)(C)C)cn1)C(=O)N(CCO)CCO ; CCCCCCCCCCCC(=O)O ; CCCCCCCCCCCC(=O)OCCN(CCOC(=O)CCCCCCCCCCC)C(=O)[C@H](Cc1cn(COCC[Si](C)(C)C)cn1)NC(=O)OC(C)(C)C ; "ZnBr2" ; CCCCCCCCCCCC(=O)OCCN(CCOC(=O)CCCCCCCCCCC)C(=O)[C@@H](N)Cc1cn(COCC[Si](C)(C)C)cn1]

To a solution of Compound 5 (1.86 g, 9.31 mmol, 2.2 eq.) in DCM (20 mL) was added DMAP (103.39 mg, 846.29 μmol, 0.2 eq.), EDCI (2.03 g, 10.58 mmol, 2.5 eq.), DIEA (1.64 g, 12.69 mmol, 2.21 mL, 3 eq.) and Compound 4 (2 g, 4.23 mmol, 1 eq.). The mixture was stirred at 20° C. for 4 hours. TLC (Dichloromethane:Methanol=10:1 R_{f}=0.6) indicated Compound 4 was consumed completely and one new spot formed. The residue was diluted with Sat. NaHCO₃ (50 mL) and extracted with DCM (100 mL). The combined organic layers were dried over Na₂SO₄, filtered and concentrated under reduced pressure to give a residue. The residue was purified by column chromatography (SiO₂, Dichloromethane:Methanol=98:2 to 94:6) to give Compound 6 (3.3 g, 3.67 mmol, 86.63% yield, 93% purity) as white solid.

Compound 6: LCMS [M+1]⁺=837.6. ¹H NMR (400 MHz, chloroform-d) δ=7.48 (s, 1H), 6.83 (s, 1H), 5.40 (br d, J=8.4 Hz, 1H), 5.18 (s, 2H), 5.02-4.82 (m, 1H), 4.26-4.18 (m, 1H), 4.18-4.03 (m, 3H), 3.89-3.68 (m, 2H), 3.66-3.53 (m, 1H), 3.46 (t, J=8.0 Hz, 2H), 3.43-3.35 (m, 1H), 3.02-2.81 (m, 2H), 2.32-2.25 (m, 4H), 1.63-1.63 (m, 1H), 1.63-1.53 (m, 4H), 1.40 (s, 9H), 1.25 (s, 32H), 0.90-0.86 (m, 6H), −0.01 (s, 9H)

### Synthesis of Compound 7:

To a solution of Compound 6 (1 g, 1.19 mmol, 1 eq.) in DCM (10 mL) was added ZnBr₂ (1.34 g, 5.97 mmol, 298.86 μL, 5 eq.). The mixture was stirred at 20° C. for 16 hours. TLC (Dichloromethane:Methanol=10:1, R_{f}=0.6) indicated Compound 6 was consumed completely and one new spot formed. The residue was diluted with Sat. NaHCO₃ (50 mL) and extracted with DCM (100 mL). The combined organic layers were dried over Na₂SO₄, filtered and concentrated under reduced pressure to give a residue. The residue was purified by column chromatography (SiO₂, Dichloromethane:Methanol=98:2 to 94:6) to give Compound 7 (500 mg, 583.34 μmol, 48.84% yield, 86% purity) as white solid.

Compound 7: LCMS [M+1]⁺=737.5. ¹H NMR (400 MHz, chloroform-d) δ=7.98 (br s, 1H), 7.02 (br s, 1H), 5.33 (s, 2H), 4.24 (br s, 5H), 3.89 (br d, J=14.4 Hz, 1H), 3.65-3.52 (m, 5H), 3.46 (br d, J=14.4 Hz, 2H), 3.05-2.83 (m, 2H), 2.41-2.27 (m, 4H), 1.61 (br s, 5H), 1.26 (s, 32H), 0.96 (t, J=7.6, 8.8 Hz, 2H), 0.90-0.84 (m, 6H), 0.06-0.01 (m, 9H).

### Synthesis of Compound 8:

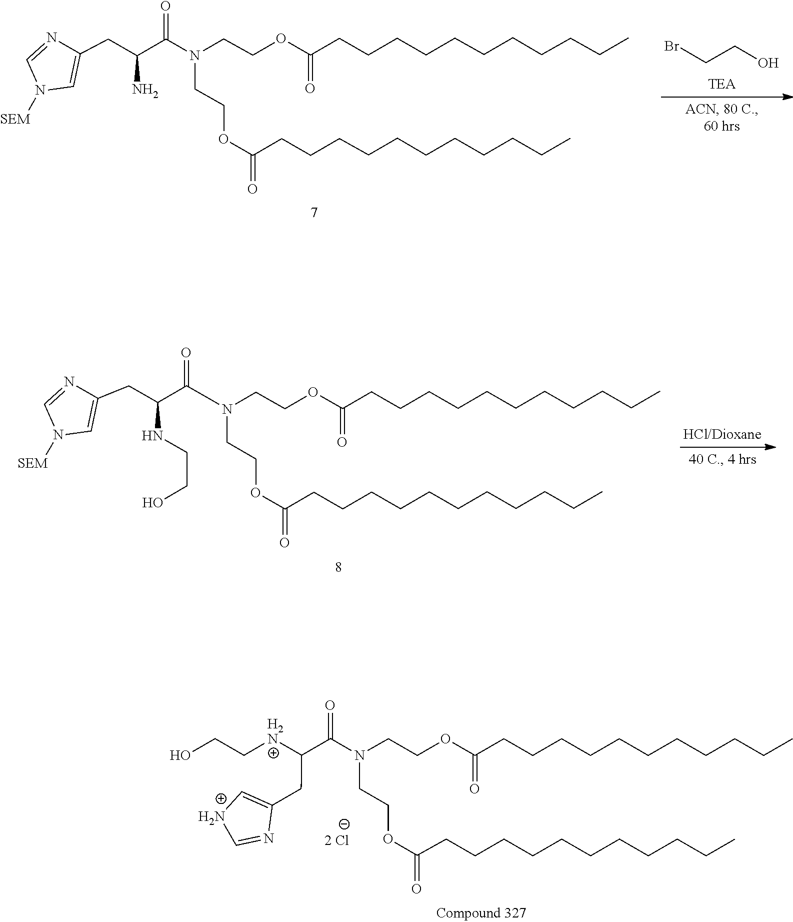

To a solution of Compound 7 (260 mg, 352.72 μmol, 1 eq.) in ACN (7 mL) was added TEA (107.07 mg, 1.06 mmol, 147.28 μL, 3 eq.) and 2-bromoethanol (132.23 mg, 1.06 mmol, 75.00 μL, 3 eq.). The mixture was stirred at 80° C. for 60 hours. LCMS showed Compound 7 was consumed completely and desired MS was detected. The reaction mixture was concentrated under reduced pressure to remove solvent. The residue was purified by column chromatography (SiO₂, DCM:MeOH=96:4 to 95:5) to give Compound 8 (200 mg, 220.18 μmol, 62.42% yield, 86% purity) as white solid.

Compound 8: LCMS [M+1]⁺=781.6. ¹H NMR (400 MHz, chloroform-d) δ=7.96 (s, 1H), 7.01 (s, 1H), 5.30 (s, 2H), 4.39-4.10 (m, 5H), 4.07-3.95 (m, 1H), 3.94-3.75 (m, 2H), 3.68-3.46 (m, 5H), 3.13-3.01 (m, 2H), 2.98 (br d, J=8.4 Hz, 1H), 2.86-2.75 (m, 1H), 2.40-2.22 (m, 4H), 1.70-1.50 (m, 4H), 1.26 (s, 32H), 1.04-0.92 (m, 2H), 0.89 (t, J=6.8 Hz, 6H), 0.08-0.01 (m, 9H).

### Synthesis of Compound 327:

A mixture of Compound 8 (500 mg, 640.05 μmol, 1 eq.) in HCl/dioxane (10 mL) was degassed and purged with N₂ for 3 times, and then the mixture was stirred at 40° C. for 4 hours under N₂ atmosphere. LCMS indicated Compound 8 was consumed completely and one main peak with desired MS was detected. The reaction mixture was concentrated under reduced pressure to give a residue. The residue was purified by prep-HPLC (column: X-Select CSH Phenyl-Hexyl 100*30 5u; mobile phase: [H₂O (0.04% HCl)-ACN]; gradient: 10%-55% B over 8.0 min) to give Compound 327 (105 mg, 161.31 μmol, 12.60% yield, HCl) as white solid.

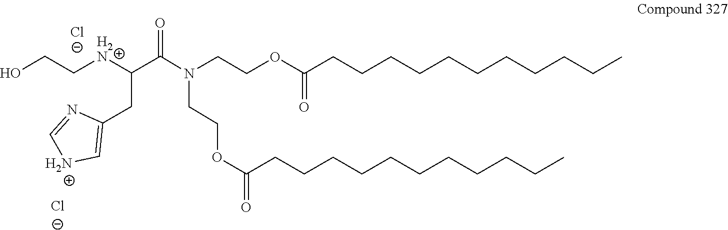

LCMS [M+1]⁺=651.6. ¹H NMR (400 MHz, DMSO-d6) δ=15.32-13.89 (m, 2H), 10.12-9.21 (m, 1H), 9.04 (m, 2H), 7.51 (s, 1H), 5.52-5.03 (m, 1H), 4.83 (br t, J=6.4 Hz, 1H), 4.21-4.02 (m, 4H), 3.84-3.73 (m, 1H), 3.71-3.64 (m, 2H), 3.63-3.58 (m, 1H), 3.58-3.50 (m, 2H), 3.31 (br d, J=6.0 Hz, 2H), 3.06-2.84 (m, 2H), 2.32-2.19 (m, 4H), 1.49 (br s, 4H), 1.32-1.18 (m, 32H), 0.92-0.79 (m, 6H).

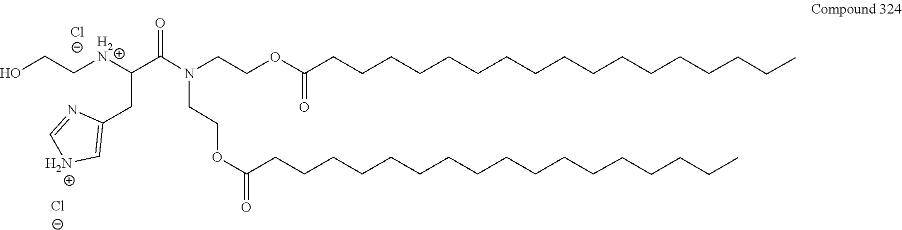

LCMS [M+1]⁺=819.7. ¹H NMR (400 MHz, DMSO-d₆) δ=14.90-13.81 (m, 2H), 9.27-8.70 (m, 2H), 8.44-8.16 (m, 1H), 7.49 (br s, 1H), 5.44-5.05 (m, 1H), 4.75 (br t, J=6.0 Hz, 1H), 4.11 (m, 4H), 3.67 (m, 8H), 3.18 (br s, 2H), 2.95 (br d, J=18.0 Hz, 2H), 2.26 (q, J=8.0 Hz, 4H), 1.48 (br s, 4H), 1.22 (s, 54H), 0.91-0.77 (m, 6H).

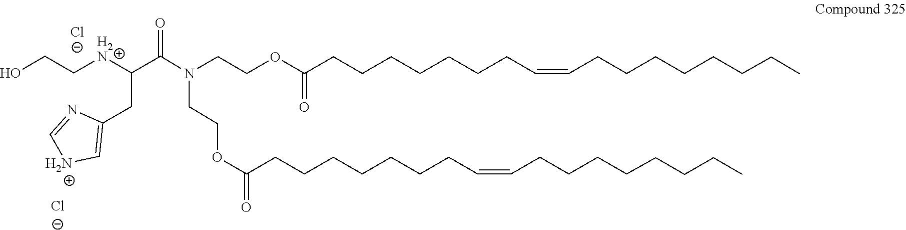

LCMS [M+1]⁺=815.7. ¹H NMR (400 MHz, DMSO-d6) δ=15.56-13.49 (m, 2H), 10.04-8.64 (m, 2H), 8.41 (s, 1H), 7.60-7.44 (m, 1H), 5.38-5.26 (m, 4H), 4.84 (t, J=6.4 Hz, 1H), 4.31-3.97 (m, 5H), 3.83-3.73 (m, 1H), 3.70-3.63 (m, 2H), 3.62-3.50 (m, 3H), 3.09-2.86 (m, 2H), 2.33-2.22 (m, 4H), 2.05-1.89 (m, 8H), 1.49 (s, 4H), 1.32-1.21 (m, 40H), 0.85 (t, J=6.8 Hz, 6H).

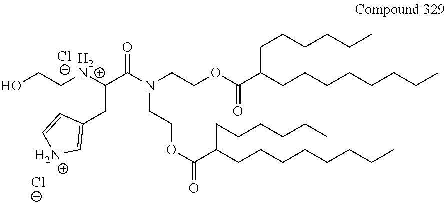

LCMS [M+1]⁺=763.7. ¹H NMR (400 MHz, DMSO-d₆) δ=15.61-14.68 (m, 1H), 14.61-13.96 (m, 1H), 9.70-9.29 (m, 1H), 9.19-8.94 (m, 1H), 8.44-8.25 (m, 1H), 7.58-7.43 (m, 1H), 5.56-5.02 (m, 1H), 4.87 (br s, 1H), 4.27-4.00 (m, 4H), 3.86-3.72 (m, 1H), 3.72-3.51 (m, 6H), 3.31 (br s, 2H), 3.04-2.86 (m, 2H), 2.28 (m, 2H), 1.47 (m, 4H), 1.41 (m, 4H), 1.21 (br s, 40H), 0.95-0.80 (m, 12H).

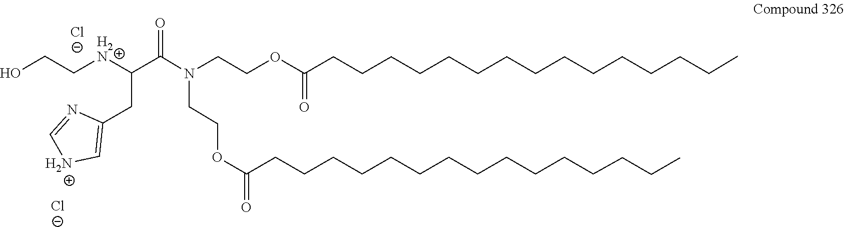

LCMS [M+1]⁺=763.6. ¹HNMR (400 MHz, DMSO-d₆) δ=14.99-14.48 (m, 1H), 14.44-13.88 (m, 1H), 9.82-8.68 (m, 2H), 8.40-8.16 (m, 1H), 7.76-7.26 (m, 1H), 5.47-5.10 (m, 1H), 4.85-4.68 (m, 1H), 4.25-3.95 (m, 4H), 3.53 (m, 8H), 3.33-3.19 (m, 2H), 3.06-2.79 (m, 2H), 2.30-2.21 (m, 4H), 1.48 (s, 4H), 1.23 (s, 46H), 0.93-0.80 (m, 6H).

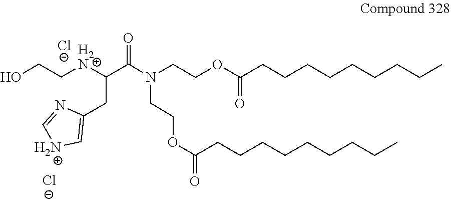

LCMS [M+1]⁺=595.5. ¹H NMR (400 MHz, DMSO-d6) δ=15.30-14.03 (m, 2H), 9.99-9.29 (m, 1H), 9.08 (s, 2H), 7.53 (s, 1H), 5.58-5.04 (m, 1H), 4.86 (br t, J=5.6 Hz, 1H), 4.20-4.01 (m, 4H), 3.88-3.74 (m, 1H), 3.68 (br t, J=5.2 Hz, 2H), 3.65-3.48 (m, 5H), 3.33 (br d, J=6.4 Hz, 2H), 3.03-2.86 (m, 2H), 2.27 (q, J=8.0 Hz, 4H), 1.54-1.43 (m, 4H), 1.23 (s, 24H), 0.85 (t, J=6.8 Hz, 6H).

### General Procedure for Preparation of Compound 323:

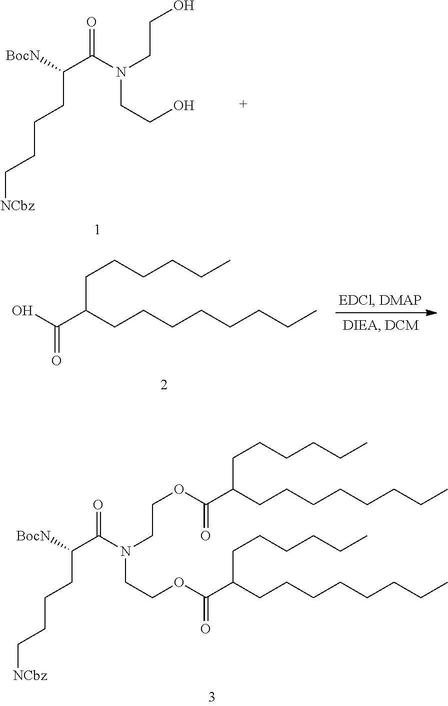

A mixture of Compound 1 (1 g, 2.14 mmol, 1 eq.), Compound 2 (1.10 g, 4.28 mmol, 2 eq.), EDCI (1.23 g, 6.42 mmol, 3 eq.), DIEA (829.27 mg, 6.42 mmol, 1.12 mL, 3 eq.) and DMAP (130.65 mg, 1.07 mmol, 0.5 eq.) in DCM (10 mL) was degassed and purged with N₂ 3 times, and the mixture was stirred at 20° C. for 16 hours under N₂ atmosphere. LCMS showed Compound 1 was consumed completely and one main peak with the desired MS was detected. The reaction mixture was partitioned between saturated NH₄Cl (30 mL) and CH₂Cl₂ (30 mL). The organic phase was separated, dried over Na₂SO₄, filtered and concentrated under reduced pressure to give a residue. The residue was purified by column chromatography (SiO₂, Petroleum ether:Ethyl acetate=30:1 to 0:1) to give Compound 3 (1.86 g, 1.95 mmol, 91.08% yield, 98.9% purity) as a white solid.

LCMS [M+1]⁺=943.6

¹H NMR (400 MHz, DMSO-d₆) δ=7.41-7.11 (m, 6H), 6.94-6.78 (m, 1H), 5.05-4.95 (m, 2H), 4.38-3.92 (m, 5H), 3.49-3.32 (m, 2H), 3.72-3.32 (m, 4H), 3.01-2.91 (m, 2H), 2.37-2.22 (m, 2H), 1.52-1.38 (m, 10H), 1.35 (s, 9H), 1.22 (s, 42H), 0.87-0.81 (m, 12H)

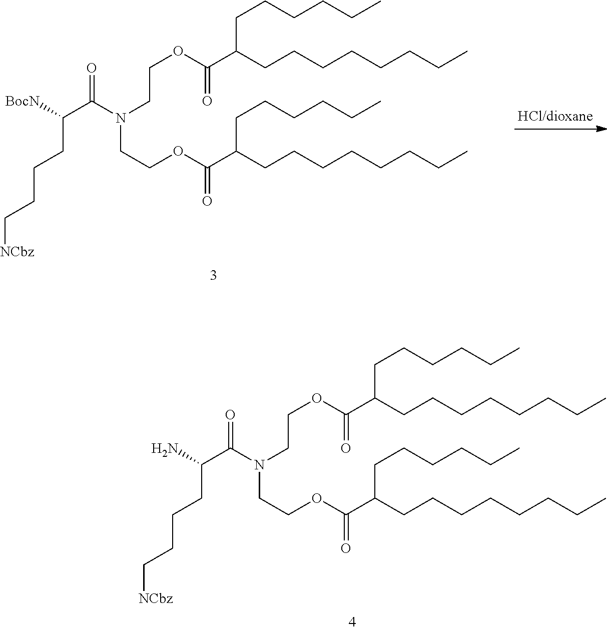

A mixture of Compound 3 (1.6 g, 2.06 mmol, 1 eq.) in HCl/dioxane (40 mL) was degassed and purged with N₂ 3 times, and the mixture was stirred at 20° C. for 2 hour under N₂ atmosphere. LCMS showed Compound 3 was consumed completely and one main peak with desired MS was detected. The reaction mixture was concentrated and lyophilized to give Compound 4 (1.4 g, 2.05 mmol, 99.46% yield, 99% purity) as yellow oil.

LCMS [M+1]⁺=845.5

¹H NMR (400 MHz, DMSO-d₆) δ=8.29-8.23 (m, 3H), 7.39-7.15 (m, 6H), 5.08-4.96 (m, 2H), 4.30-4.05 (m, 5H), 3.90-3.81 (m, 1H), 3.74-3.64 (m, 1H), 3.59-3.47 (m, 1H), 3.39-3.34 (m, 1H), 3.04-2.92 (m, 2H), 2.19 (m, 4H), 1.73-1.63 (m, 2H), 1.55-1.46 (m, 4H), 1.43-1.31 (m, 4H), 1.29-1.20 (m, 24H), 0.90-0.78 (m, 6H)

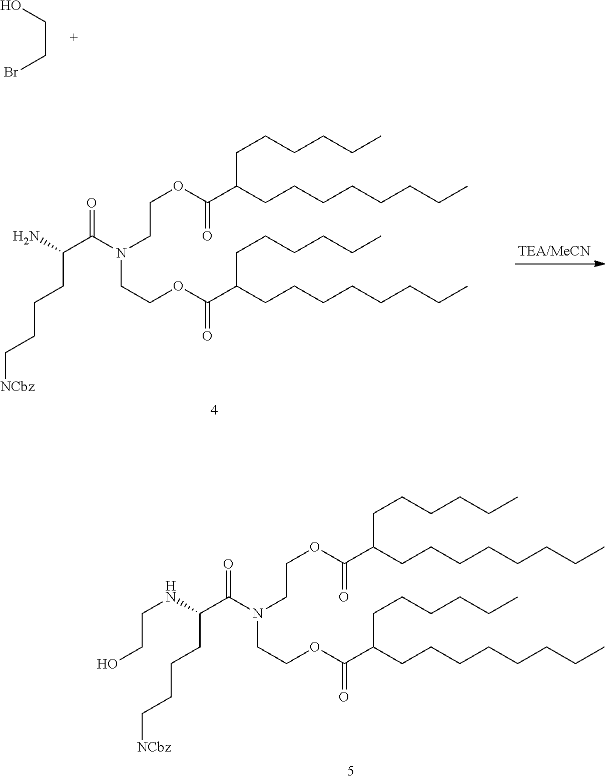

To a solution of Compound 4 (410 mg, 575.52 μmol, 1 eq., HCl) in MeCN (2.5 mL) was added 2-bromoethanol (215.76 mg, 1.73 mmol, 122.38 μL, 3 eq.) and TEA (174.71 mg, 1.73 mmol, 240.32 μL, 3 eq.) at 25° C. The resulting mixture was heated to 80° C. for 16 hours. Then, 2-bromoethanol (35.96 mg, 287.76 μmol, 20.40 μL, 0.5 eq.) was added, and the resulting mixture was stirred at 80° C. for 16 hours. LCMS showed desired MS was detected.

The mixture was concentrated to give a residue. The residue was purified by prep-TLC (SiO₂, DCM:MeOH=10:1) to give Compound 5 (0.22 g, 233.69 μmol, 40.61% yield, 76.48% purity) as a colorless oil.

LCMS [M+1]⁺=888.5

¹H NMR (400 MHz, DMSO-d₆) δ=7.28 (br s, 5H), 7.21 (br t, J=5.6 Hz, 1H), 5.05-4.92 (m, 2H), 4.46-4.36 (m, 1H), 4.27-3.99 (m, 4H), 3.75-3.60 (m, 2H), 3.60-3.32 (m, 5H), 3.00-2.90 (m, 2H), 2.45-2.31 (m, 2H), 2.31-2.20 (m, 4H), 1.54-1.39 (m, 6H), 1.38-1.33 (m, 2H), 1.27-1.19 (m, 26H), 0.88-0.82 (m, 6H)

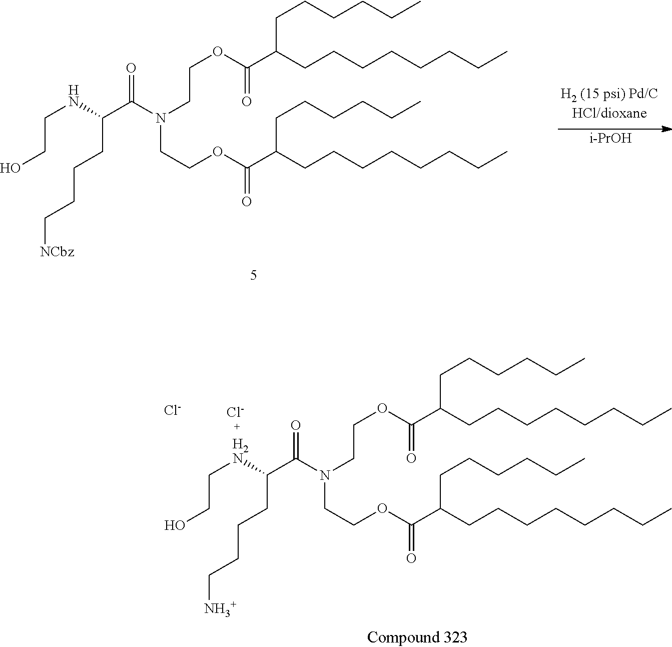

To a solution of Compound 5 (170 mg, 191.38 μmol, 1 eq.) in isopropanol (15 mL) was added Pd/C (170.00 mg, 159.74 μmol, 10% purity, 8.35e-1 eq.) and HCl/dioxane (0.5 M, 842.05 μL, 2.2 eq.). The mixture was degassed and purged with H₂ 3 times, and the mixture was stirred at 20° C. for 2 hours under H₂ atmosphere. TLC (Dichloromethane:Methanol=10:1, P:R_{f}=0) indicated that Compound 5 was consumed and one new spot formed. The reaction mixture was filtered and lyophilized to give Compound 323 (120 mg, 151.78 μmol, 79.31% yield, HCl) as a light yellow solid.

LCMS [M+1]⁺=754.7

¹H NMR (400 MHz, DMSO-d₆) δ=9.77-9.12 (m, 1H), 9.04-8.46 (m, 1H), 8.27-7.62 (m, 3H), 5.22 (m, 1H), 4.63-4.29 (m, 1H), 4.28-4.03 (m, 4H), 3.86-3.43 (m, 6H), 3.06-2.80 (m, 2H), 2.74 (s, 2H), 2.37-2.36 (m, 1H), 2.37-2.22 (m, 1H), 1.80 (s, 2H), 1.60-1.33 (m, 12H), 1.23 (s, 40H), 0.85 (t, J=6.4 Hz, 12H)

### General Procedure for Preparation of Compound 340

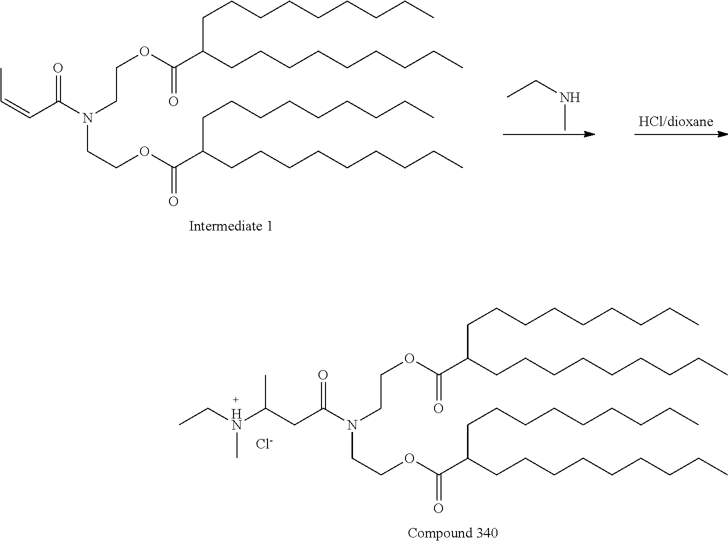

To a solution of Intermediate 1 (1 eq.) in THE (1 mL) and MeOH (1 mL) is added ethylmethylamine (2 M, 20 eq.). The reaction mixture is stirred at 70° C. overnight. TLC indicates the extent of the reaction and the mixture is concentrated under reduced pressure to give a residue. The residue is purified by column chromatography to give Compound 340.

To a solution of this residue in DCM (1 mL) is added HCl/dioxane (1.5 mL, 1 M) at 25° C. The reaction mixture is stirred at 25° C. for 1 hour. The reaction mixture is concentrated and lyophilized to give Compound 340 in HCl salt form.

### General Procedure for Preparation of Compound 341

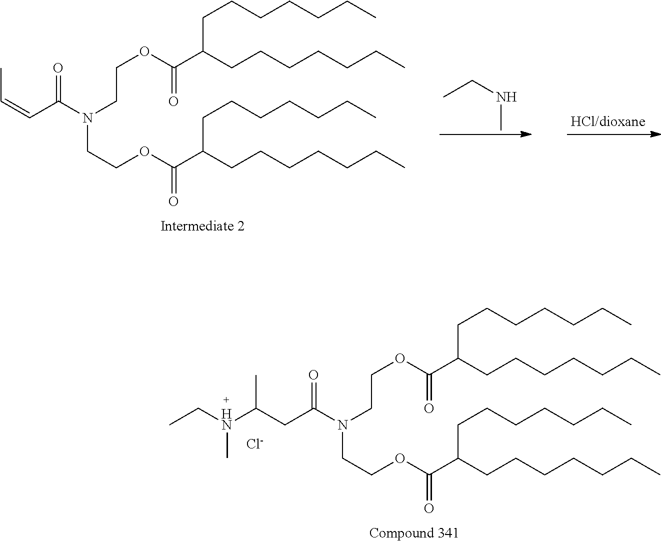

To a solution of Intermediate 2 (1 eq.) in THE (1 mL) and MeOH (1 mL) is added ethylmethylamine (2 M, 20 eq.). The reaction mixture is stirred at 70° C. overnight. TLC indicates the extent of the reaction and the mixture is concentrated under reduced pressure to give a residue. The residue is purified by column chromatography to give Compound 341.

To a solution of this residue in DCM (1 mL) is added HCl/dioxane (1.5 mL, 1 M) at 25° C. The reaction mixture is stirred at 25° C. for 1 hour. The reaction mixture is concentrated and lyophilized to give Compound 341 in HCl salt form.

### General Procedure for Preparation of Compound 342

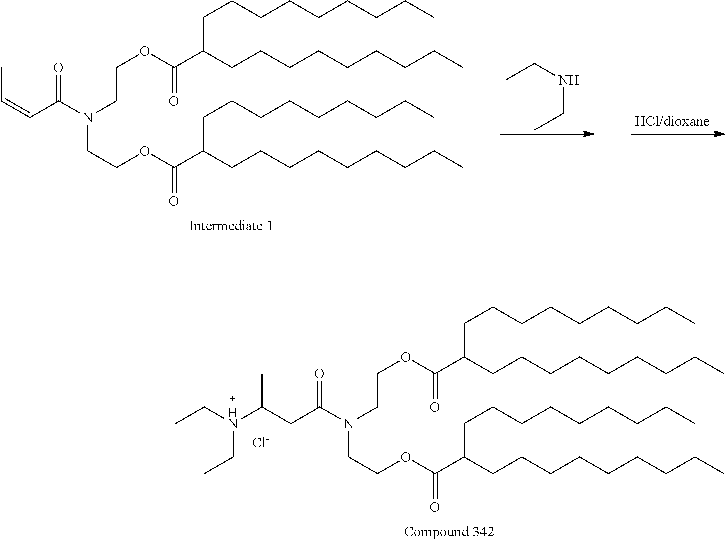

To a solution of Intermediate 1 (1 eq.) in THE (1 mL) and MeOH (1 mL) is added diethylamine (2 M, 20 eq.). The reaction mixture is stirred at 70° C. overnight. TLC indicates the extent of the reaction and the mixture is concentrated under reduced pressure to give a residue. The residue is purified by column chromatography to give Compound 342.

To a solution of this residue in DCM (1 mL) is added HCl/dioxane (1.5 mL, 1 M) at 25° C. The reaction mixture is stirred at 25° C. for 1 hour. The reaction mixture is concentrated and lyophilized to give Compound 342 in HCl salt form.

### General Procedure for Preparation of Compound 343

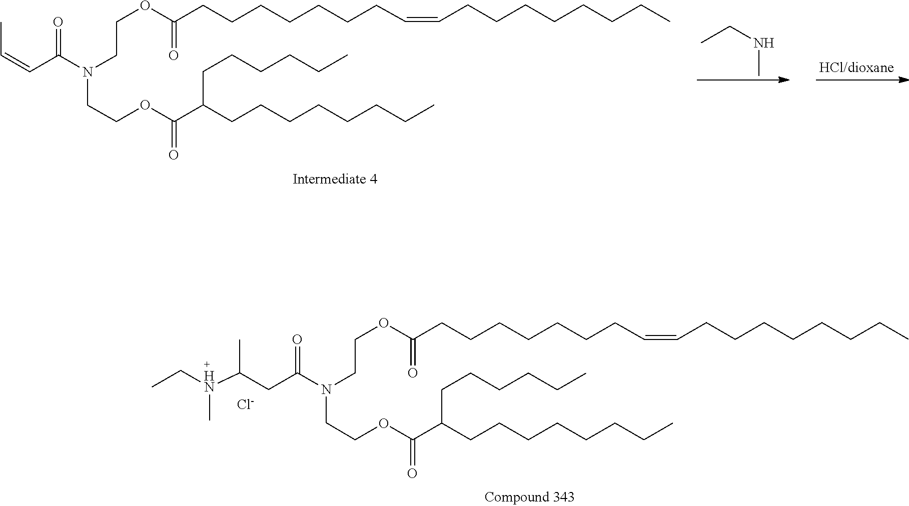

To a solution of Intermediate 4 (1 eq.) in THF (1 mL) and MeOH (1 mL) is added ethylmethylamine (2 M, 20 eq.). The reaction mixture is stirred at 70° C. overnight. TLC indicates the extent of the reaction and the mixture is concentrated under reduced pressure to give a residue. The residue is purified by column chromatography to give Compound 343

To a solution of this residue in DCM (1 mL) is added HCl/dioxane (1.5 mL, 1 M) at 25° C. The reaction mixture is stirred at 25° C. for 1 hour. The reaction mixture is concentrated and lyophilized to give Compound 343 in HCl salt form.

### General Procedure for Preparation of Compound 344

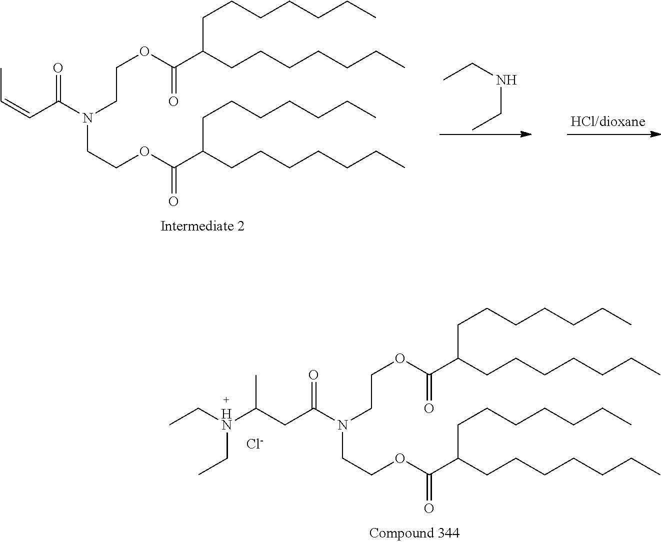

To a solution of Intermediate 2 (1 eq.) in THF (1 mL) and MeOH (1 mL) is added diethylamine (2 M, 20 eq.). The reaction mixture is stirred at 70° C. overnight. TLC indicates the extent of the reaction and the mixture is concentrated under reduced pressure to give a residue. The residue is purified by column chromatography to give Compound 344.

To a solution of this residue in DCM (1 mL) is added HCl/dioxane (1.5 mL, 1 M) at 25° C. The reaction mixture is stirred at 25° C. for 1 hour. The reaction mixture is concentrated and lyophilized to give Compound 344 in HCl salt form.

### General Procedure for Preparation of Compound 345

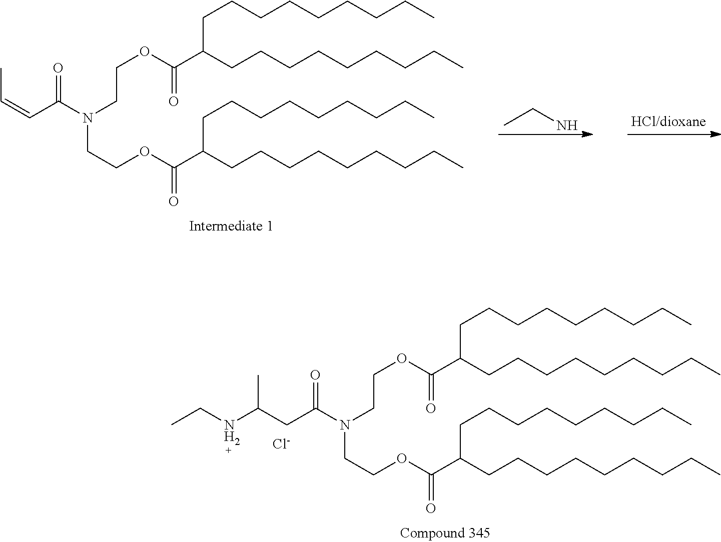

To a solution of Intermediate 1 (1 eq.) in THE (1 mL) and MeOH (1 mL) is added ethylamine (2 M, 20 eq.). The reaction mixture is stirred at 70° C. overnight. TLC indicates the extent of the reaction and the mixture is concentrated under reduced pressure to give a residue. The residue is purified by column chromatography to give Compound 345.

To a solution of this residue in DCM (1 mL) is added HCl/dioxane (1.5 mL, 1 M) at 25° C. The reaction mixture is stirred at 25° C. for 1 hour. The reaction mixture is concentrated and lyophilized to give Compound 345 in HCl salt form.

### General Procedure for Preparation of Compound 346

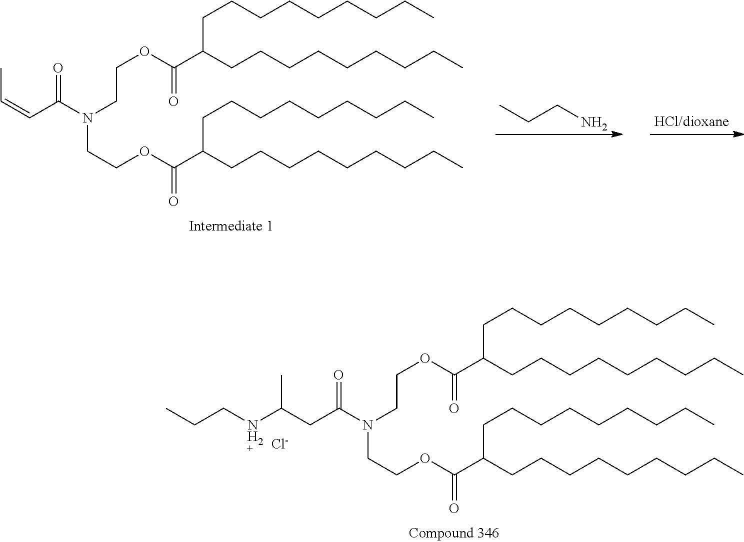

To a solution of Intermediate 1 (1 eq.) in THE (1 mL) and MeOH (1 mL) is added propylamine (2 M, 20 eq.). The reaction mixture is stirred at 70° C. overnight. TLC indicates the extent of the reaction and the mixture is concentrated under reduced pressure to give a residue. The residue is purified by column chromatography to give Compound 346.

To a solution of this residue in DCM (1 mL) is added HCl/dioxane (1.5 mL, 1 M) at 25° C. The reaction mixture is stirred at 25° C. for 1 hour. The reaction mixture is concentrated and lyophilized to give Compound 346 in HCl salt form.

### General Procedure for Preparation of Compound 347

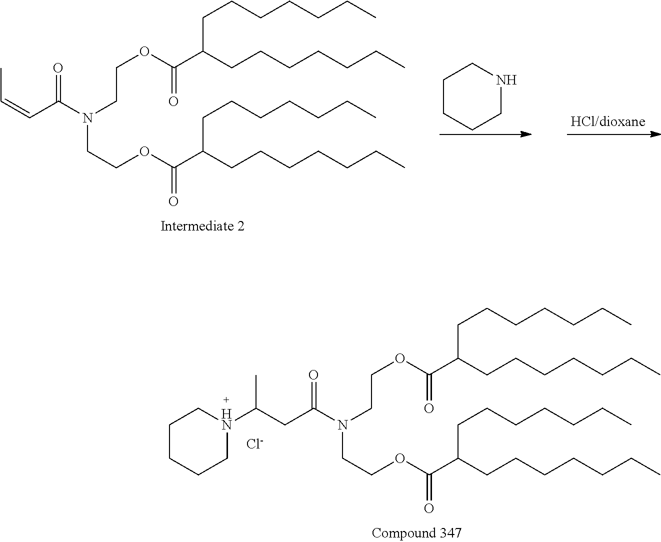

To a solution of Intermediate 2 (1 eq.) in THF (1 mL) and MeOH (1 mL) is added cyclohexylamine (2 M, 20 eq.). The reaction mixture is stirred at 70° C. overnight. TLC indicates the extent of the reaction and the mixture is concentrated under reduced pressure to give a residue. The residue is purified by column chromatography to give Compound 347.

To a solution of this residue in DCM (1 mL) is added HCl/dioxane (1.5 mL, 1 M) at 25° C. The reaction mixture is stirred at 25° C. for 1 hour. The reaction mixture is concentrated and lyophilized to give Compound 347 in HCl salt form.

### General Procedure for Preparation of Compound 348

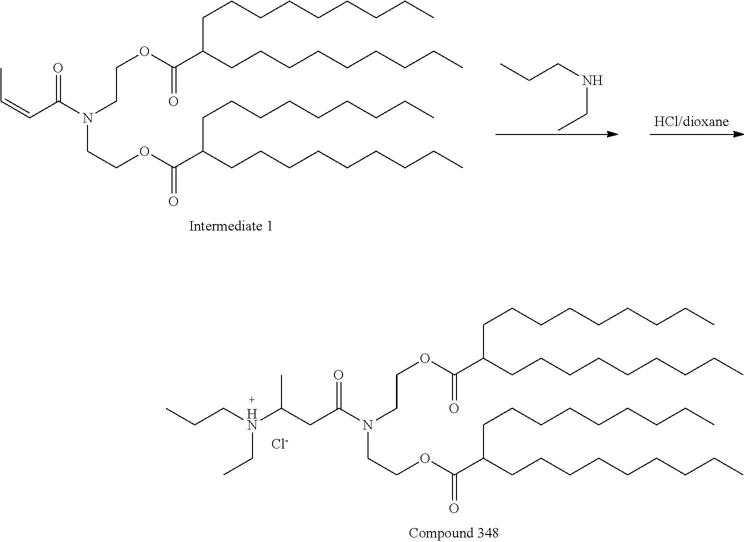

To a solution of Intermediate 1 (1 eq.) in THF (1 mL) and MeOH (1 mL) is added ethylpropylamine (2 M, 20 eq.). The reaction mixture is stirred at 70° C. overnight. TLC indicates the extent of the reaction and the mixture is concentrated under reduced pressure to give a residue. The residue is purified by column chromatography to give Compound 348.

To a solution of this residue in DCM (1 mL) is added HCl/dioxane (1.5 mL, 1 M) at 25° C. The reaction mixture is stirred at 25° C. for 1 hour. The reaction mixture is concentrated and lyophilized to give Compound 348 in HCl salt form.

### General Procedure for Preparation of Compound 349

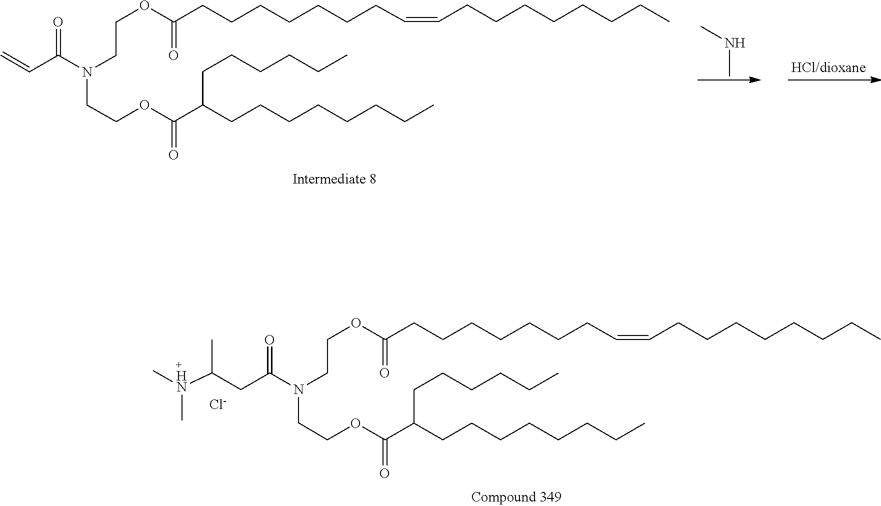

To a solution of Intermediate 8 (1 eq.) in THF (1 mL) and MeOH (1 mL) is added dimethylamine (2 M, 20 eq.). The reaction mixture is stirred at 70° C. overnight. TLC indicates the extent of the reaction and the mixture is concentrated under reduced pressure to give a residue. The residue is purified by column chromatography to give Compound 349.

To a solution of this residue in DCM (1 mL) is added HCl/dioxane (1.5 mL, 1 M) at 25° C. The reaction mixture is stirred at 25° C. for 1 hour. The reaction mixture is concentrated and lyophilized to give Compound 349 in HCl salt form.

### General Procedure for Preparation of Compound 350

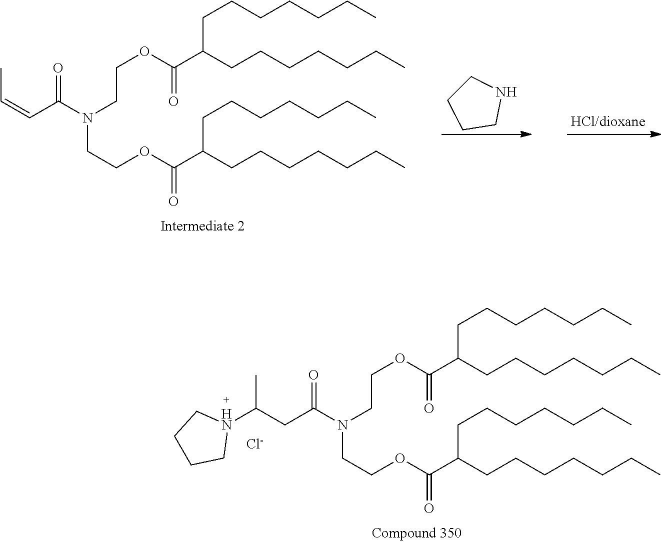

To a solution of Intermediate 2 (1 eq.) in THE (1 mL) and MeOH (1 mL) is added cyclopentylamine (2 M, 20 eq.). The reaction mixture is stirred at 70° C. overnight. TLC indicates the extent of the reaction and the mixture is concentrated under reduced pressure to give a residue. The residue is purified by column chromatography to give Compound 350.

To a solution of this residue in DCM (1 mL) is added HCl/dioxane (1.5 mL, 1 M) at 25° C. The reaction mixture is stirred at 25° C. for 1 hour. The reaction mixture is concentrated and lyophilized to give Compound 350 in HCl salt form.

### General Procedure for Preparation of Compound 351

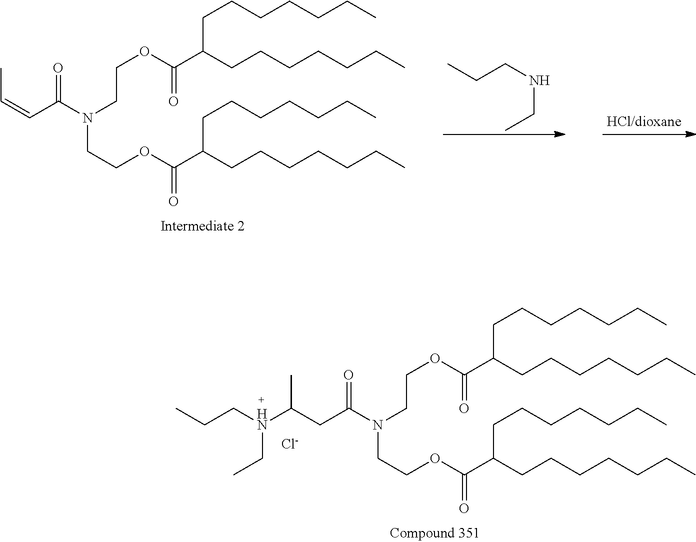

To a solution of Intermediate 2 (1 eq.) in THE (1 mL) and MeOH (1 mL) is added ethylpropylamine (2 M, 20 eq.). The reaction mixture is stirred at 70° C. overnight. TLC indicates the extent of the reaction and the mixture is concentrated under reduced pressure to give a residue. The residue is purified by column chromatography to give Compound 351.

To a solution of this residue in DCM (1 mL) is added HCl/dioxane (1.5 mL, 1 M) at 25° C. The reaction mixture is stirred at 25° C. for 1 hour. The reaction mixture is concentrated and lyophilized to give Compound 351 in HCl salt form.

### General Procedure for Preparation of Compound 352

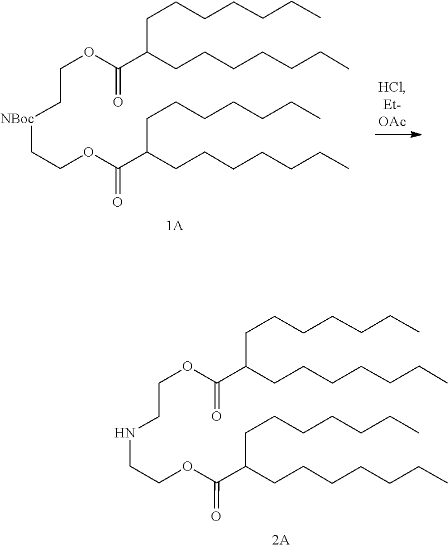 [SMILES: CCCCCCCC(CCCCCCC)C(=O)OCCN(CCOC(=O)C(CCCCCCC)CCCCCCC)C(=O)OC(C)(C)C ; CCCCCCCC(CCCCCCC)C(=O)OCCNCCOC(=O)C(CCCCCCC)CCCCCCC]

A solution of Compound 1A (1 eq.) in HCl/EtOAc (13.64 eq.) is stirred at 20° C. for 2 hours. LC-MS indicates whether Compound 1A has been consumed and the target MS detected.

The reaction mixture is concentrated under reduced pressure to give Compound 2A.

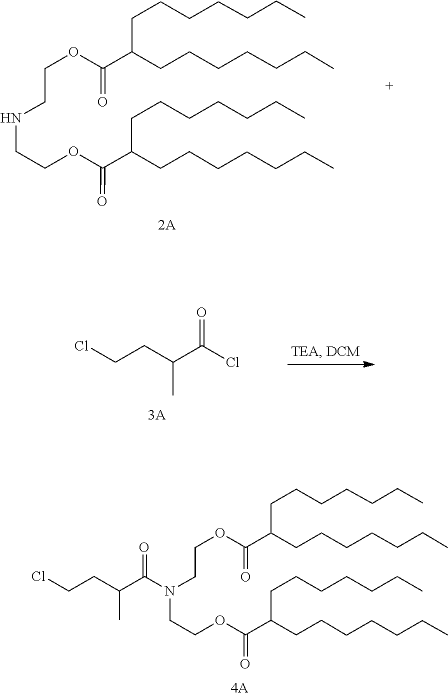

To a solution of Compound 2A (1 eq., HCl salt) in DCM (100 mL) is added TEA (5 eq.) and Compound 3A (2 eq.) at −20° C. The reaction mixture is stirred at 25° C. overnight. TLC indicates whether Compound 2A is consumed completely and new spots formed. The mixture is concentrated under reduced pressure to give a residue. The residue is purified by column chromatography to give Compound 4A.

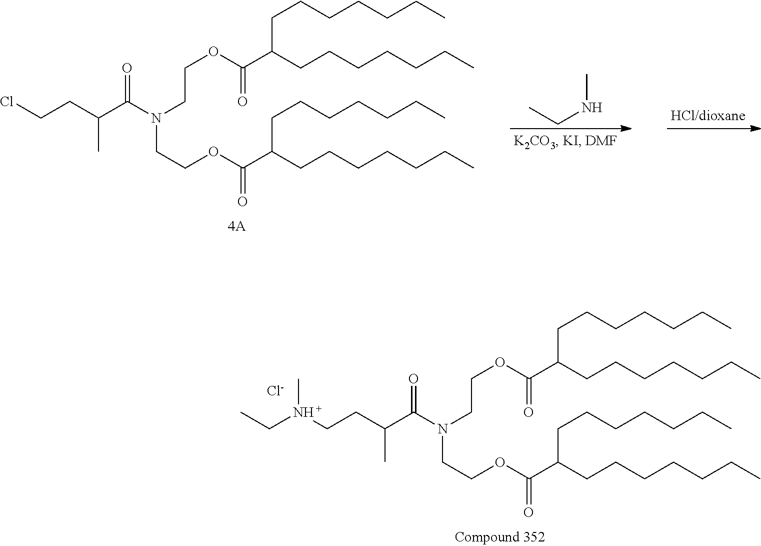

To a solution of Compound 4A (1 eq.) in in DMF (3 mL) is added K₂CO₃ (5 eq.), KI (2 eq.) and ethylmethylamine (20 eq.). The reaction mixture is stirred at 70° C. for 12 hours. TLC indicates whether Compound 4A has been consumed completely and new spots formed. The mixture is concentrated under reduced pressure to give a residue. The residue is purified by column chromatography to give Compound 352.

To a solution of this residue in DCM (1 mL) is added HCl/dioxane (1.5 mL, 1 M) at 25° C. The reaction mixture is stirred at 25° C. for 1 hour. The reaction mixture is concentrated and lyophilized to give Compound 352 in HCl salt form.

### General Procedure for Preparation of Compound 353

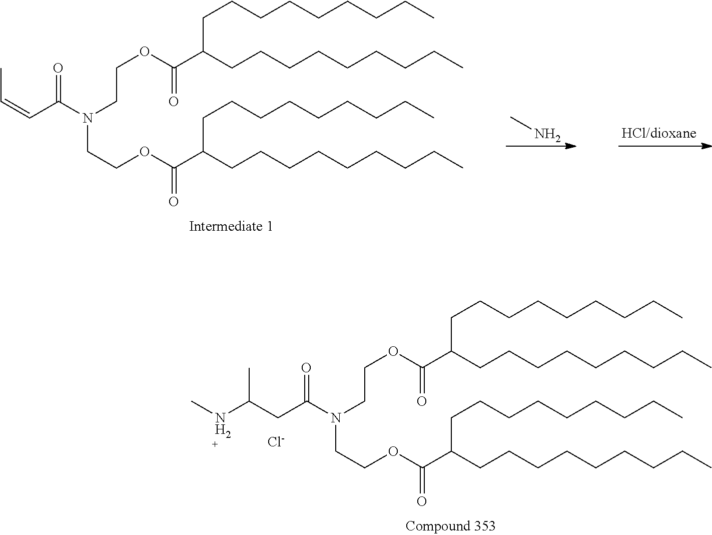

To a solution of Intermediate 1 (1 eq.) in THE (1 mL) and MeOH (1 mL) is added methylamine (2 M, 20 eq.). The reaction mixture is stirred at 70° C. overnight. TLC indicates the extent of the reaction and the mixture is concentrated under reduced pressure to give a residue. The residue is purified by column chromatography to give Compound 353.

To a solution of this residue in DCM (1 mL) is added HCl/dioxane (1.5 mL, 1 M) at 25° C. The reaction mixture is stirred at 25° C. for 1 hour. The reaction mixture is concentrated and lyophilized to give Compound 353 in HCl salt form.

### General Procedure for Preparation of Compound 354

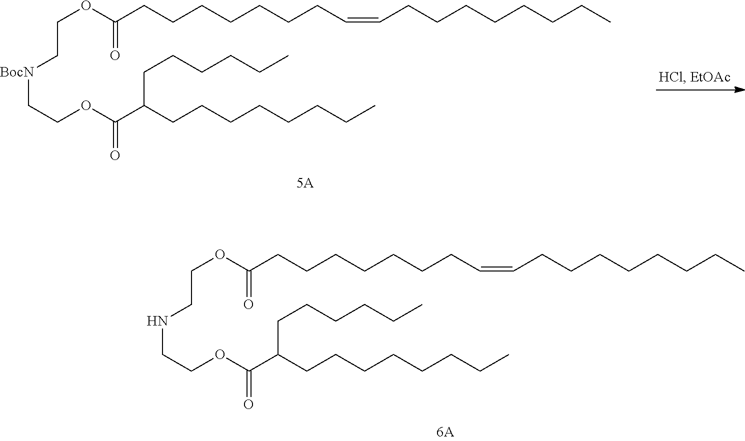 [SMILES: CCCCCCCC/C=C\CCCCCCCC(=O)OCCN(CCOC(=O)C(CCCCCC)CCCCCCCC)C(=O)OC(C)(C)C ; CCCCCCCC/C=C\CCCCCCCC(=O)OCCNCCOC(=O)C(CCCCCC)CCCCCCCC]

A solution of Compound 5A (1 eq.) in HCl/EtOAc (13.64 eq.) is stirred at 20° C. for 2 hours. LC-MS indicates whether Compound 5A has been consumed and the target MS detected. The reaction mixture is concentrated under reduced pressure to give Compound 6A.

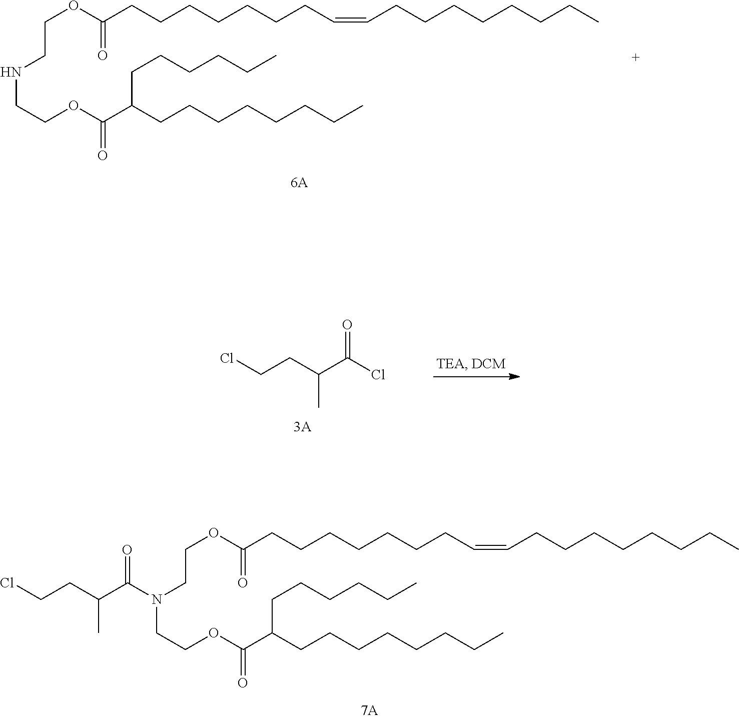

To a solution of Compound 6A (1 eq., HCl salt) in DCM (100 mL) is added TEA (5 eq.) and Compound 3A (2 eq.) at −20° C. The reaction mixture is stirred at 25° C. overnight. TLC indicates whether Compound 7A is consumed completely and new spots formed. The mixture is concentrated under reduced pressure to give a residue. The residue is purified by column chromatography to give Compound 7A.

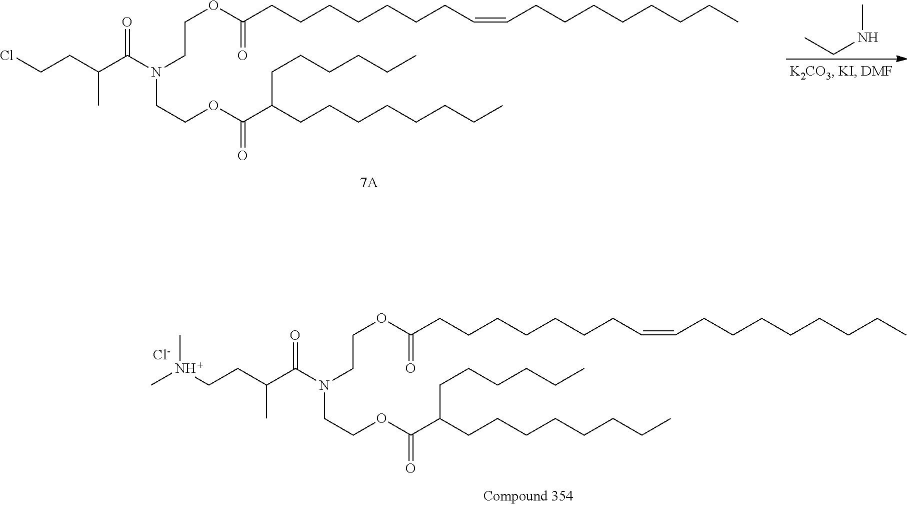

To a solution of Compound 7A (1 eq.) in in DMF (3 mL) is added K₂CO₃ (5 eq.), KI (2 eq.) and ethylmethylamine (20 eq.). The reaction mixture is stirred at 70° C. for 12 hours. TLC indicates whether Compound 7A has been consumed completely and new spots formed. The mixture is concentrated under reduced pressure to give a residue. The residue is purified by column chromatography to give Compound 354.

### General Procedure for Preparation of Compound 355

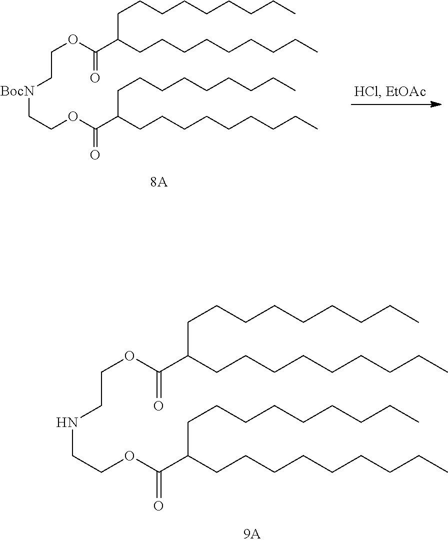 [SMILES: CCCCCCCCCC(CCCCCCCCC)C(=O)OCCN(CCOC(=O)C(CCCCCCCCC)CCCCCCCCC)C(=O)OC(C)(C)C ; CCCCCCCCCC(CCCCCCCCC)C(=O)OCCNCCOC(=O)C(CCCCCCCCC)CCCCCCCCC]

A solution of 8A (1 eq.) in HCl/EtOAc (13.64 eq.) is stirred at 20° C. for 1 hour. LCMS determines whether 8A has been consumed completely and if a single main peak with desired mass is detected. The mixture is concentrated to give Compound 9A.

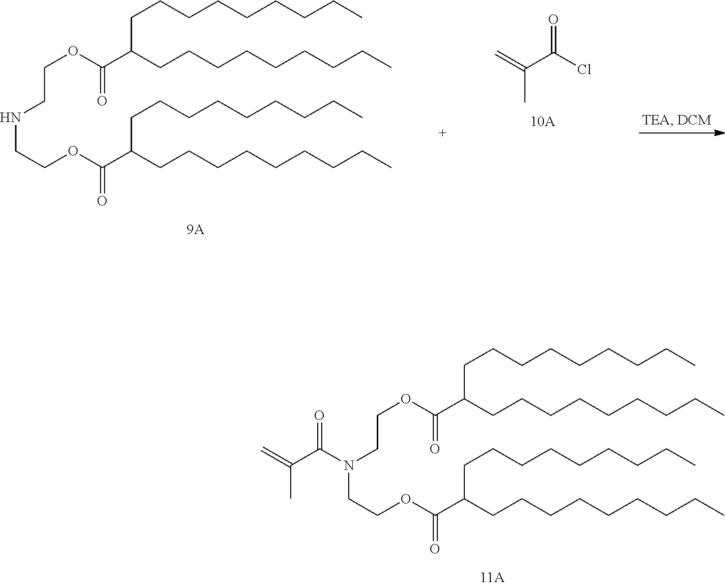

To a mixture of 9A (1 eq.) and TEA (, 20 eq.) in DCM (10 mL) is added 10A (1.5 eq.) at 0° C. The mixture is stirred at 20° C. for 16 hours. LCMS determines whether 10A has been consumed completely and if a single main peak with desired mass is detected. The reaction mixture is diluted with sat.NH₄Cl (20 mL) and extracted with DCM (60 mL). The combined organic layers re dried over Na₂SO₄, filtered and concentrated under reduced pressure to give a residue. The residue is purified by column chromatography to give 11A.

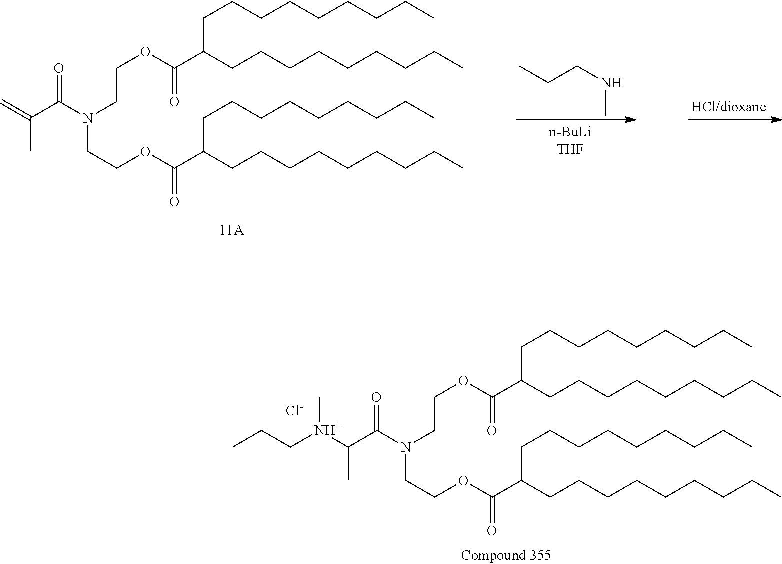

To a solution of methylpropylamine (5 eq.) in THF (3 mL) is added n-BuLi (3 eq.) at −70° C. for 30 min. Then, 11A (1 eq.) in THF (0.5 mL) is added dropwise at −70° C. The mixture is stirred at −70° C. for 2 hours. LCMS determines whether 11A has been consumed completely and if a single main peak with desired mass is detected. The reaction mixture is quenched by addition of NH₄Cl (5 mL) at −70° C., and extracted with DCM (24 mL). The combined organic layers are washed with brine (5 mL), dried over Na₂SO₄, filtered and concentrated under reduced pressure to give a residue. The residue is purified by column chromatography to give Compound 355

To a solution of this residue in DCM (1 mL) is added HCl/dioxane (1.5 mL, 1 M) at 25° C. The reaction mixture is stirred at 25° C. for 1 hour. The reaction mixture is concentrated and lyophilized to give Compound 355 in HCl salt form.

### General Procedure for Preparation of Compound 356

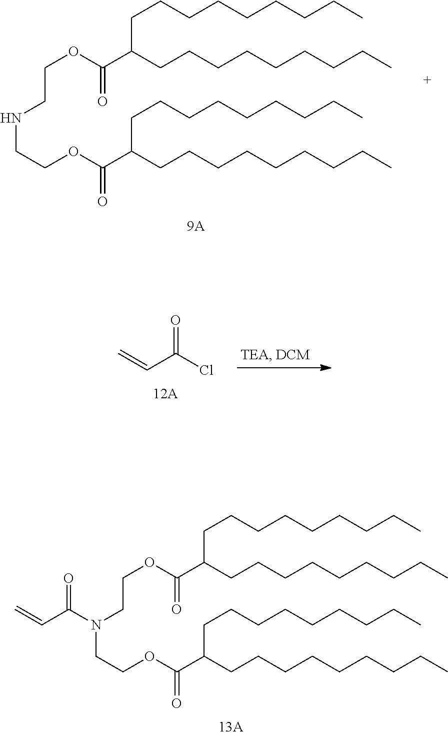

To a mixture of 9A (1 eq.) and TEA (, 20 eq.) in DCM (10 mL) is added 12A (1.5 eq.) at 0° C. The mixture is stirred at 20° C. for 16 hours. LCMS determines whether 12A has been consumed completely and if a single main peak with desired mass is detected. The reaction mixture is diluted with sat.NH₄Cl (20 mL) and extracted with DCM (60 mL). The combined organic layers re dried over Na₂SO₄, filtered and concentrated under reduced pressure to give a residue. The residue is purified by column chromatography to give 13A.

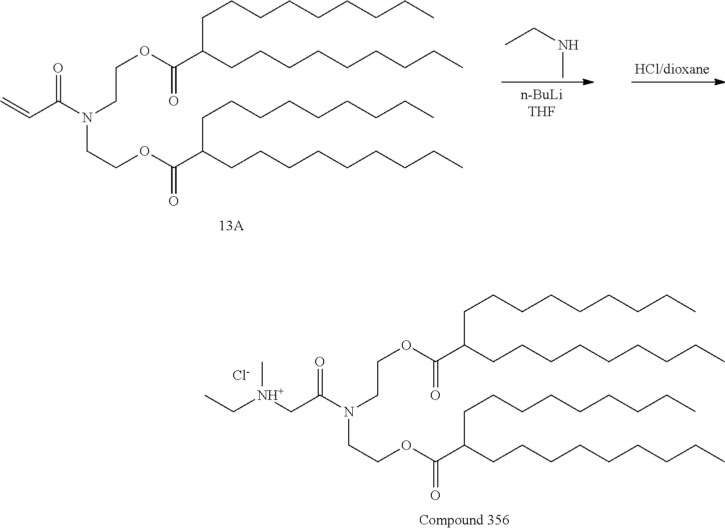

To a solution of ethylmethylamine (5 eq.) in THF (3 mL) is added n-BuLi (3 eq.) at −70° C. for 30 min. Then, 13A (1 eq.) in THF (0.5 mL) is added dropwise at −70° C. The mixture is stirred at −70° C. for 2 hours. LCMS determines whether 13A has been consumed completely and if a single main peak with desired mass is detected. The reaction mixture is quenched by addition of NH₄Cl (5 mL) at −70° C., and extracted with DCM (24 mL). The combined organic layers are washed with brine (5 mL), dried over Na₂SO₄, filtered and concentrated under reduced pressure to give a residue. The residue is purified by column chromatography to give Compound 356

To a solution of this residue in DCM (1 mL) is added HCl/dioxane (1.5 mL, 1 M) at 25° C. The reaction mixture is stirred at 25° C. for 1 hour. The reaction mixture is concentrated and lyophilized to give Compound 356 in HCl salt form.

### General Procedure for Preparation of Compound 357

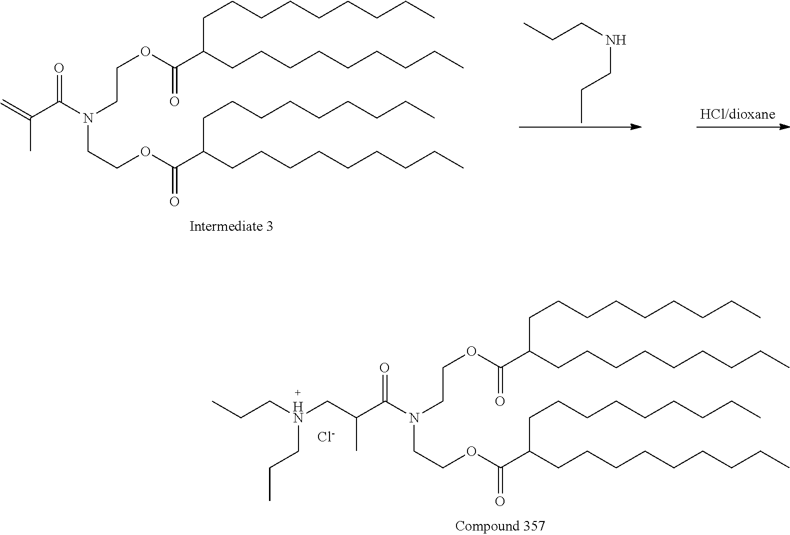

To a solution of Intermediate 3 (1 eq.) in THF (1 mL) and MeOH (1 mL) is added dipropylamine (2 M, 20 eq.). The reaction mixture is stirred at 70° C. overnight. TLC indicates the extent of the reaction and the mixture is concentrated under reduced pressure to give a residue. The residue is purified by column chromatography to give Compound 357.

To a solution of this residue in DCM (1 mL) is added HCl/dioxane (1.5 mL, 1 M) at 25° C. The reaction mixture is stirred at 25° C. for 1 hour. The reaction mixture is concentrated and lyophilized to give Compound 357 in HCl salt form.

### General Procedure for Preparation of Compound 358

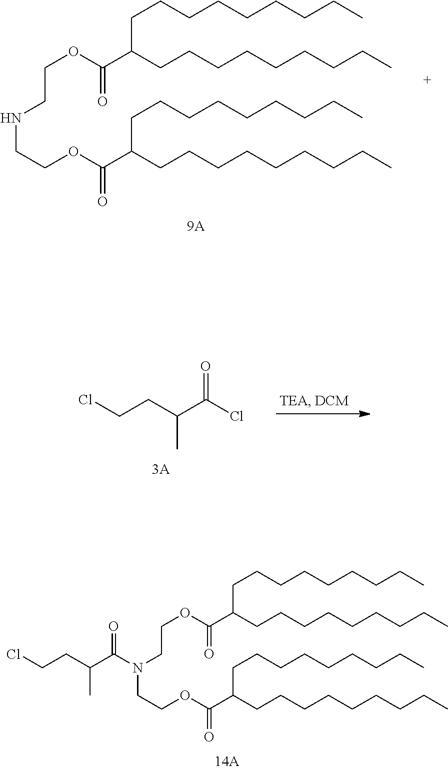

To a solution of Compound 9A (1 eq., HCl salt) in DCM (100 mL) is added TEA (5 eq.) and Compound 3A (2 eq.) at −20° C. The reaction mixture is stirred at 25° C. overnight. TLC indicates whether Compound 9A is consumed completely and new spots formed. The mixture is concentrated under reduced pressure to give a residue. The residue is purified by column chromatography to give Compound 14A.

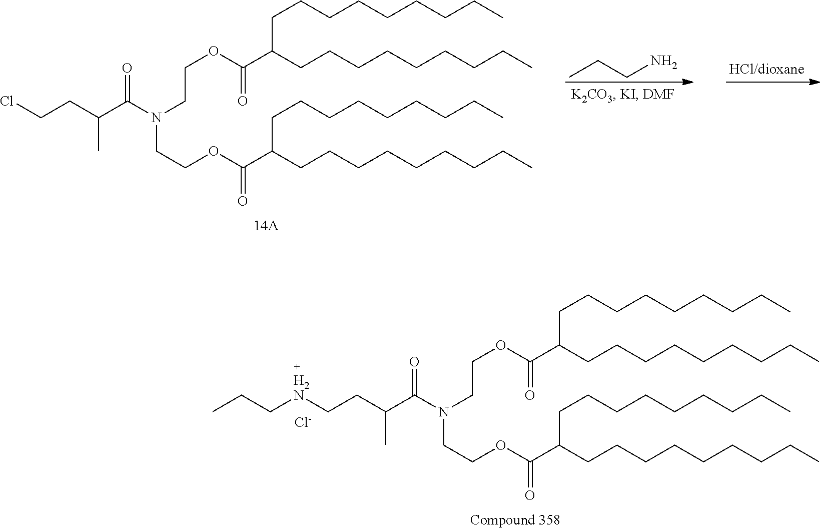

To a solution of Compound 14A (1 eq.) in in DMF (3 mL) is added K₂CO₃ (5 eq.), KI (2 eq.) and propylamine (20 eq.). The reaction mixture is stirred at 70° C. for 12 hours. TLC indicates whether Compound 14A has been consumed completely and new spots formed. The mixture is concentrated under reduced pressure to give a residue. The residue is purified by column chromatography to give Compound 358.

To a solution of this residue in DCM (1 mL) is added HCl/dioxane (1.5 mL, 1 M) at 25° C. The reaction mixture is stirred at 25° C. for 1 hour. The reaction mixture is concentrated and lyophilized to give Compound 358 in HCl salt form.

### General Procedure for Preparation of Compound 359

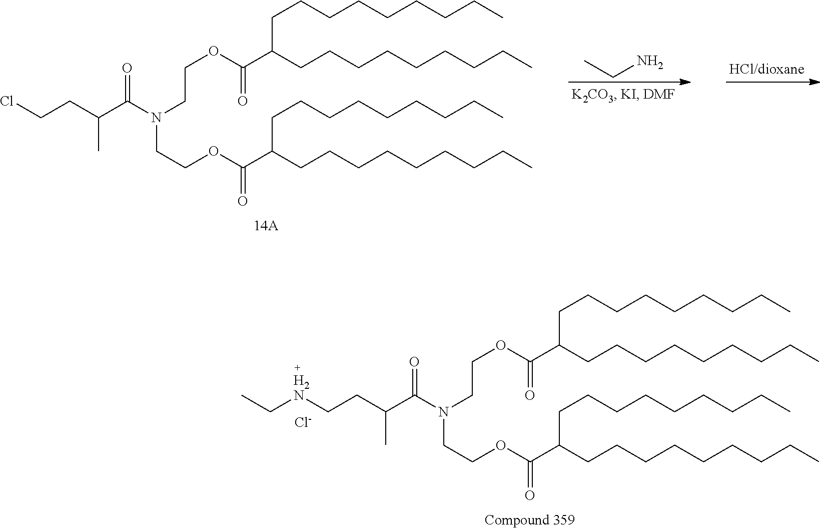

To a solution of Compound 14A (1 eq.) in in DMF (3 mL) is added K₂CO₃ (5 eq.), KI (2 eq.) and ethylamine (20 eq.). The reaction mixture is stirred at 70° C. for 12 hours. TLC indicates whether Compound 14A has been consumed completely and new spots formed. The mixture is concentrated under reduced pressure to give a residue. The residue is purified by column chromatography to give Compound 359.

To a solution of this residue in DCM (1 mL) is added HCl/dioxane (1.5 mL, 1 M) at 25° C. The reaction mixture is stirred at 25° C. for 1 hour. The reaction mixture is concentrated and lyophilized to give Compound 359 in HCl salt form.

### General Procedure for Preparation of Compound 360

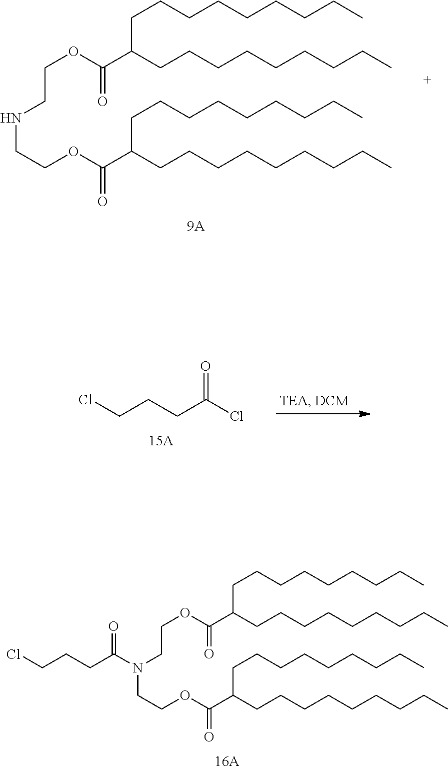 [SMILES: CCCCCCCCCC(CCCCCCCCC)C(=O)OCCNCCOC(=O)C(CCCCCCCCC)CCCCCCCCC ; O=C(Cl)CCCCl ; CCCCCCCCCC(CCCCCCCCC)C(=O)OCCN(CCOC(=O)C(CCCCCCCCC)CCCCCCCCC)C(=O)CCCCl]

To a solution of Compound 9A (1 eq., HCl salt) in DCM (100 mL) is added TEA (5 eq.) and Compound 15A (2 eq.) at −20° C. The reaction mixture is stirred at 25° C. overnight. TLC indicates whether Compound 9A is consumed completely and new spots formed. The mixture is concentrated under reduced pressure to give a residue. The residue is purified by column chromatography to give Compound 16A.

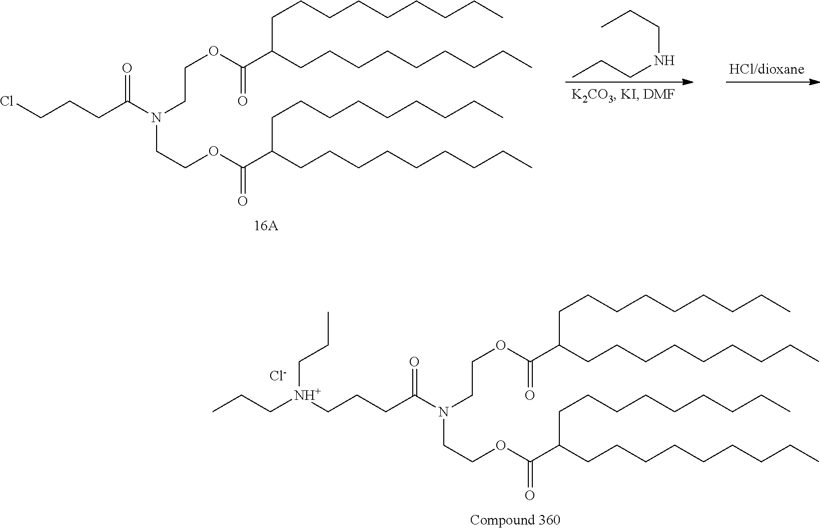

To a solution of Compound 16A (1 eq.) in in DMF (3 mL) is added K₂CO₃ (5 eq.), KI (2 eq.) and dipropylamine (20 eq.). The reaction mixture is stirred at 70° C. for 12 hours. TLC indicates whether Compound 16A has been consumed completely and new spots formed. The mixture is concentrated under reduced pressure to give a residue. The residue is purified by column chromatography to give Compound 360.

To a solution of this residue in DCM (1 mL) is added HCl/dioxane (1.5 mL, 1 M) at 25° C. The reaction mixture is stirred at 25° C. for 1 hour. The reaction mixture is concentrated and lyophilized to give Compound 360 in HCl salt form.

### General Procedure for Preparation of Compound 361

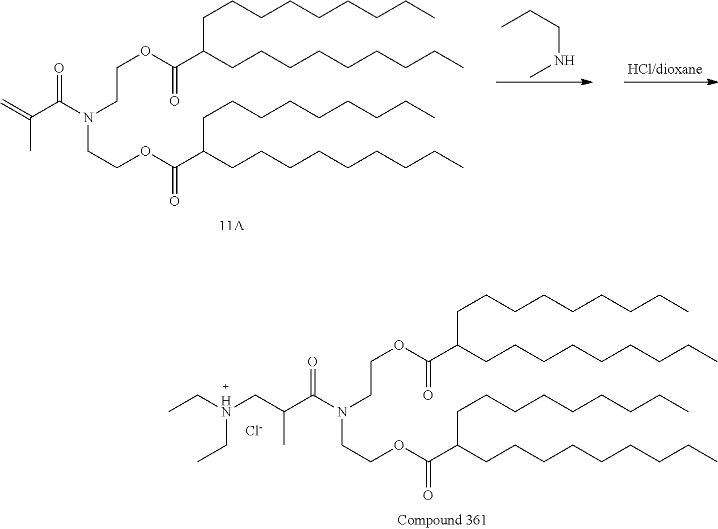

To a solution of 11A (1 eq.) in THF (1 mL) and MeOH (1 mL) is added diethylamine (2 M, 20 eq.). The reaction mixture is stirred at 70° C. overnight. TLC indicates the extent of the reaction and the mixture is concentrated under reduced pressure to give a residue. The residue is purified by column chromatography to give Compound 361.

To a solution of this residue in DCM (1 mL) is added HCl/dioxane (1.5 mL, 1 M) at 25° C. The reaction mixture is stirred at 25° C. for 1 hour. The reaction mixture is concentrated and lyophilized to give Compound 361 in HCl salt form.

### General Procedure for Preparation of Compound 362

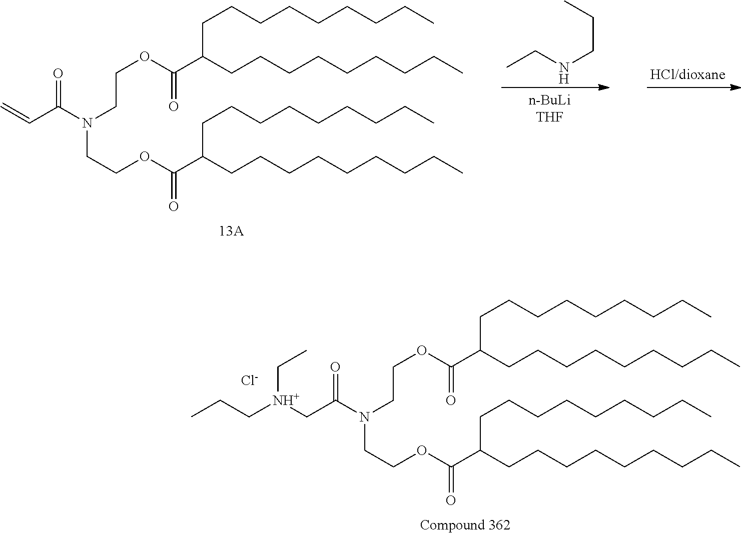

To a solution of ethylpropylamine (5 eq.) in THE (3 mL) is added n-BuLi (3 eq.) at −70° C. for 30 min. Then, 13A (1 eq.) in THF (0.5 mL) is added dropwise at −70° C. The mixture is stirred at −70° C. for 2 hours. LCMS determines whether 13A has been consumed completely and if a single main peak with desired mass is detected. The reaction mixture is quenched by addition of NH₄Cl (5 mL) at −70° C., and extracted with DCM (24 mL). The combined organic layers are washed with brine (5 mL), dried over Na₂SO₄, filtered and concentrated under reduced pressure to give a residue. The residue is purified by column chromatography to give Compound 362

To a solution of this residue in DCM (1 mL) is added HCl/dioxane (1.5 mL, 1 M) at 25° C. The reaction mixture is stirred at 25° C. for 1 hour. The reaction mixture is concentrated and lyophilized to give Compound 362 in HCl salt form.

### General Procedure for Preparation of Compound 363

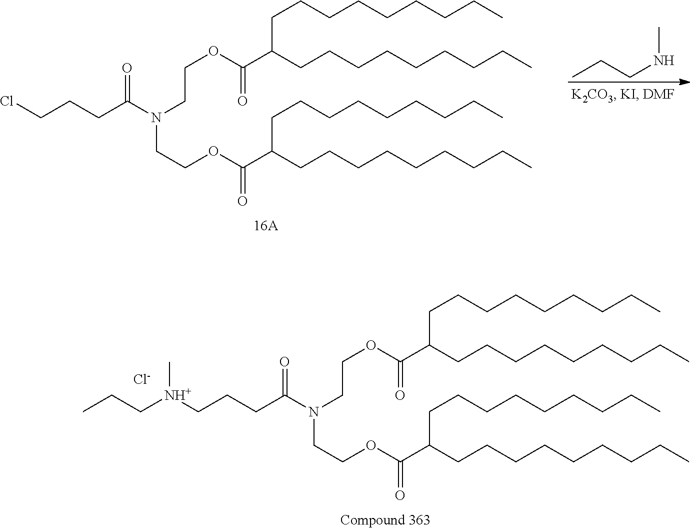

To a solution of Compound 16A (1 eq.) in in DMF (3 mL) is added K₂CO₃ (5 eq.), KI (2 eq.) and ethylpropylamine (20 eq.). The reaction mixture is stirred at 70° C. for 12 hours.

TLC indicates whether Compound 14A has been consumed completely and new spots formed. The mixture is concentrated under reduced pressure to give a residue. The residue is purified by column chromatography to give Compound 363.

To a solution of this residue in DCM (1 mL) is added HCl/dioxane (1.5 mL, 1 M) at 25° C. The reaction mixture is stirred at 25° C. for 1 hour. The reaction mixture is concentrated and lyophilized to give Compound 363 in HCl salt form.

### General Procedure for Preparation of Compound 364

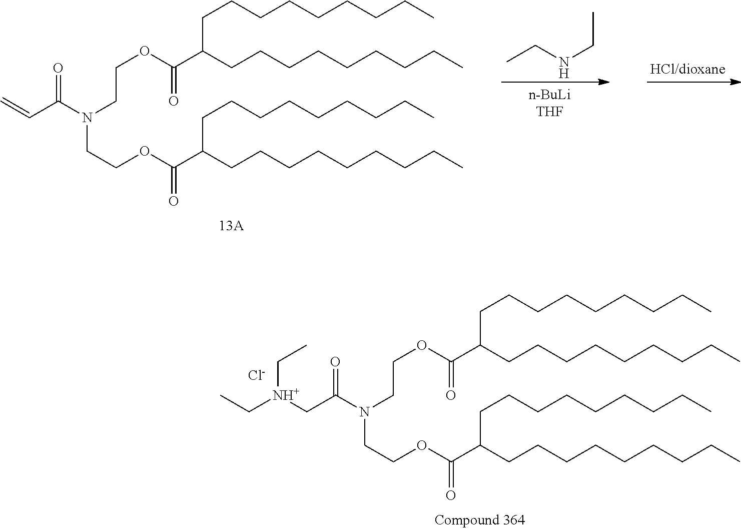

To a solution of diethylamine (5 eq.) in THF (3 mL) is added n-BuLi (3 eq.) at −70° C. for 30 min. Then, 13A (1 eq.) in THF (0.5 mL) is added dropwise at −70° C. The mixture is stirred at −70° C. for 2 hours. LCMS determines whether 13A has been consumed completely and if a single main peak with desired mass is detected. The reaction mixture is quenched by addition of NH₄Cl (5 mL) at −70° C., and extracted with DCM (24 mL). The combined organic layers are washed with brine (5 mL), dried over Na₂SO₄, filtered and concentrated under reduced pressure to give a residue. The residue is purified by column chromatography to give Compound 364.

To a solution of this residue in DCM (1 mL) is added HCl/dioxane (1.5 mL, 1 M) at 25° C. The reaction mixture is stirred at 25° C. for 1 hour. The reaction mixture is concentrated and lyophilized to give Compound 364 in HCl salt form.

### General Procedure for Preparation of Compound 365

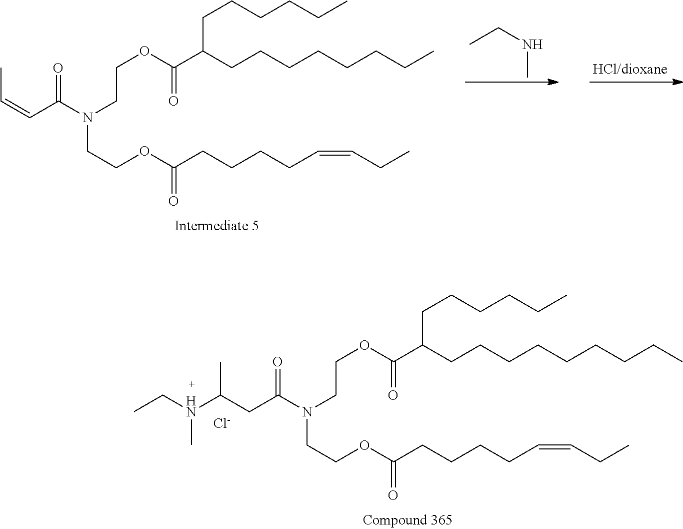

To a solution of Intermediate 5 (1 eq.) in THF (1 mL) and MeOH (1 mL) is added ethylmethylamine (2 M, 20 eq.). The reaction mixture is stirred at 70° C. overnight. TLC indicates the extent of the reaction and the mixture is concentrated under reduced pressure to give a residue. The residue is purified by column chromatography to give Compound 365.

To a solution of this residue in DCM (1 mL) is added HCl/dioxane (1.5 mL, 1 M) at 25° C. The reaction mixture is stirred at 25° C. for 1 hour. The reaction mixture is concentrated and lyophilized to give Compound 365 in HCl salt form.

### General Procedure for Preparation of Compound 366

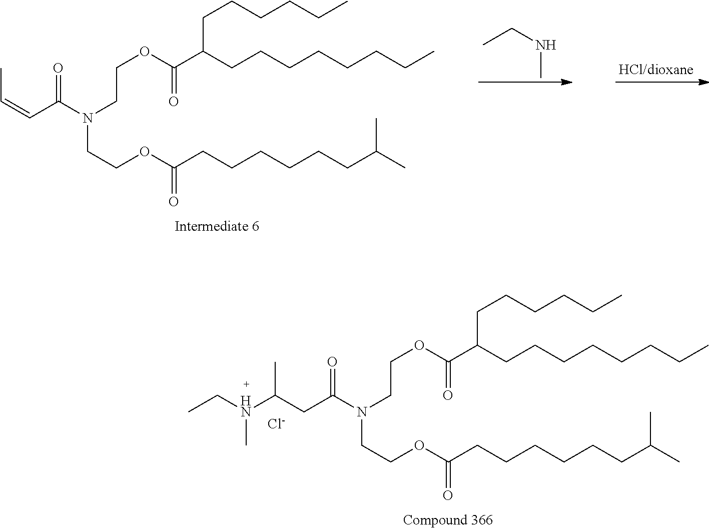

To a solution of Intermediate 6 (1 eq.) in THE (1 mL) and MeOH (1 mL) is added ethylmethylamine (2 M, 20 eq.). The reaction mixture is stirred at 70° C. overnight. TLC indicates the extent of the reaction and the mixture is concentrated under reduced pressure to give a residue. The residue is purified by column chromatography to give Compound 366.

To a solution of this residue in DCM (1 mL) is added HCl/dioxane (1.5 mL, 1 M) at 25° C. The reaction mixture is stirred at 25° C. for 1 hour. The reaction mixture is concentrated and lyophilized to give Compound 366 in HCl salt form.

### General Procedure for Preparation of Compound 367

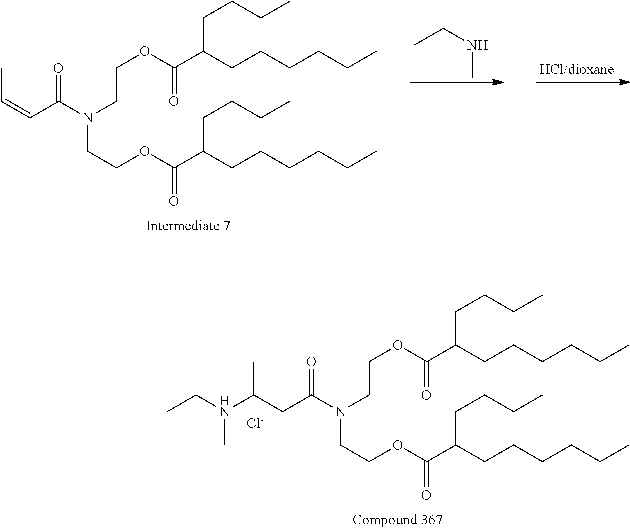

To a solution of Intermediate 7 (1 eq.) in THF (1 mL) and MeOH (1 mL) is added ethylmethylamine (2 M, 20 eq.). The reaction mixture is stirred at 70° C. overnight. TLC indicates the extent of the reaction and the mixture is concentrated under reduced pressure to give a residue. The residue is purified by column chromatography to give Compound 367.

To a solution of this residue in DCM (1 mL) is added HCl/dioxane (1.5 mL, 1 M) at 25° C. The reaction mixture is stirred at 25° C. for 1 hour. The reaction mixture is concentrated and lyophilized to give Compound 367 in HCl salt form.

### General Procedure for Preparation of Compound 368

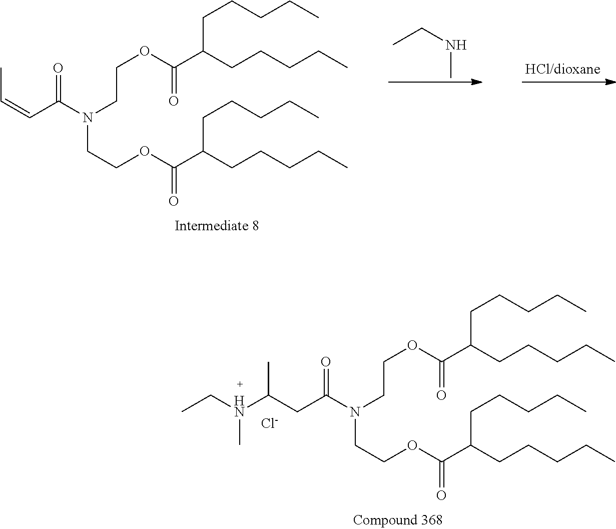

To a solution of Intermediate 8 (1 eq.) in THF (1 mL) and MeOH (1 mL) is added ethylmethylamine (2 M, 20 eq.). The reaction mixture is stirred at 70° C. overnight. TLC indicates the extent of the reaction and the mixture is concentrated under reduced pressure to give a residue. The residue is purified by column chromatography to give Compound 368.

To a solution of this residue in DCM (1 mL) is added HCl/dioxane (1.5 mL, 1 M) at 25° C. The reaction mixture is stirred at 25° C. for 1 hour. The reaction mixture is concentrated and lyophilized to give Compound 368 in HCl salt form.

### General Procedure for Preparation of Compound 369

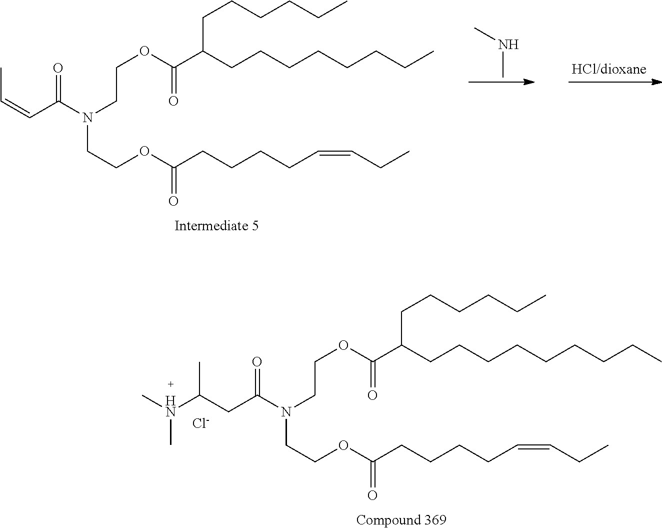

To a solution of Intermediate 5 (1 eq.) in THF (1 mL) and MeOH (1 mL) is added dimethylamine (2 M, 20 eq.). The reaction mixture is stirred at 70° C. overnight. TLC indicates the extent of the reaction and the mixture is concentrated under reduced pressure to give a residue. The residue is purified by column chromatography to give Compound 369.

To a solution of this residue in DCM (1 mL) is added HCl/dioxane (1.5 mL, 1 M) at 25° C. The reaction mixture is stirred at 25° C. for 1 hour. The reaction mixture is concentrated and lyophilized to give Compound 369 in HCl salt form.

### General Procedure for Preparation of Compound 370

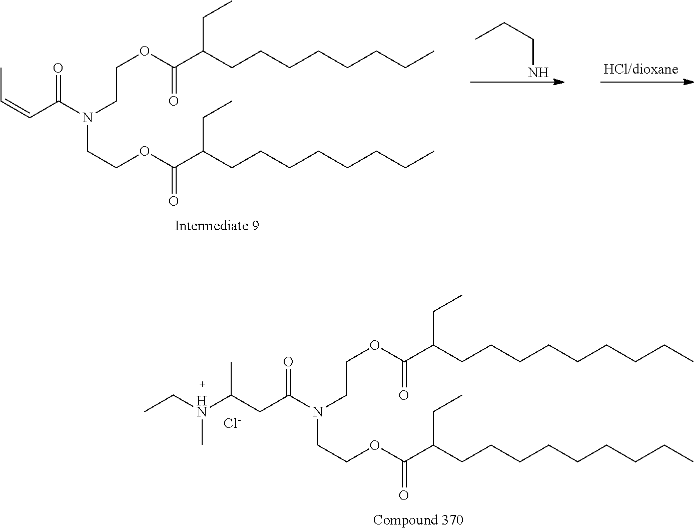

To a solution of Intermediate 9 (1 eq.) in THE (1 mL) and MeOH (1 mL) is added ethylmethylamine (2 M, 20 eq.). The reaction mixture is stirred at 70° C. overnight. TLC indicates the extent of the reaction and the mixture is concentrated under reduced pressure to give a residue. The residue is purified by column chromatography to give Compound 370.

To a solution of this residue in DCM (1 mL) is added HCl/dioxane (1.5 mL, 1 M) at 25° C. The reaction mixture is stirred at 25° C. for 1 hour. The reaction mixture is concentrated and lyophilized to give Compound 370 in HCl salt form.

### General Procedure for Preparation of Compound 371

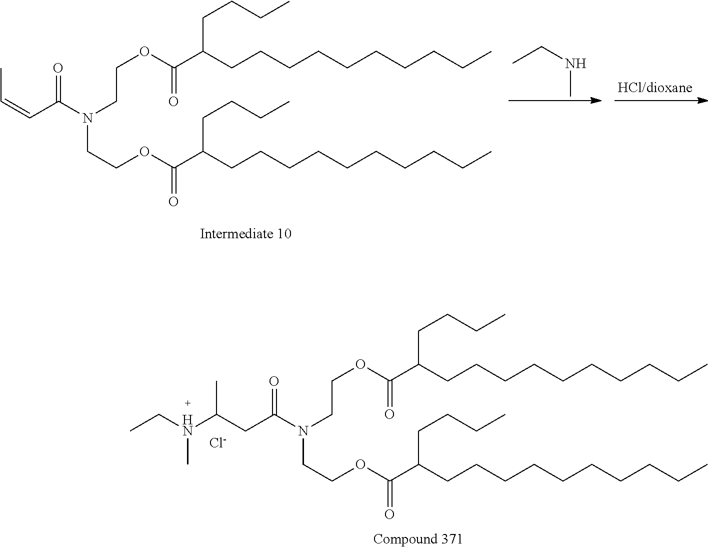

To a solution of Intermediate 10 (1 eq.) in THF (1 mL) and MeOH (1 mL) is added ethylmethylamine (2 M, 20 eq.). The reaction mixture is stirred at 70° C. overnight. TLC indicates the extent of the reaction and the mixture is concentrated under reduced pressure to give a residue. The residue is purified by column chromatography to give Compound 371.

To a solution of this residue in DCM (1 mL) is added HCl/dioxane (1.5 mL, 1 M) at 25° C. The reaction mixture is stirred at 25° C. for 1 hour. The reaction mixture is concentrated and lyophilized to give Compound 371 in HCl salt form.

### General Procedure for Preparation of Compound 372

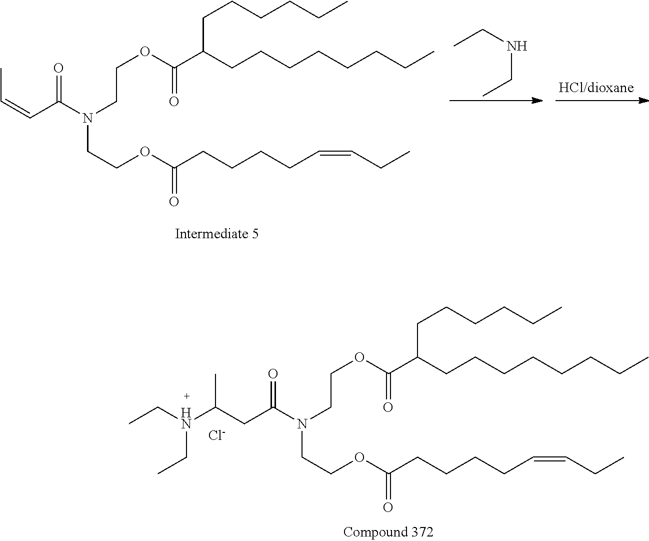

To a solution of Intermediate 5 (1 eq.) in THF (1 mL) and MeOH (1 mL) is added diethylamine (2 M, 20 eq.). The reaction mixture is stirred at 70° C. overnight. TLC indicates the extent of the reaction and the mixture is concentrated under reduced pressure to give a residue. The residue is purified by column chromatography to give Compound 372.

To a solution of this residue in DCM (1 mL) is added HCl/dioxane (1.5 mL, 1 M) at 25° C. The reaction mixture is stirred at 25° C. for 1 hour. The reaction mixture is concentrated and lyophilized to give Compound 372 in HCl salt form.

### General Procedure for Preparation of Compound 373

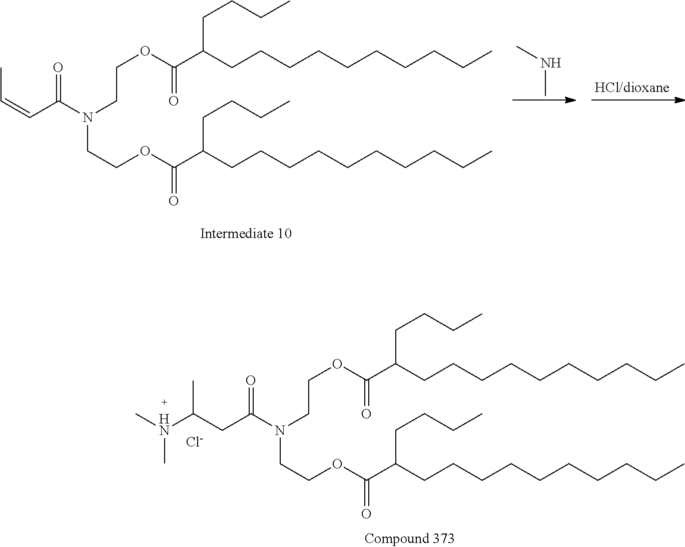

To a solution of Intermediate 10 (1 eq.) in THF (1 mL) and MeOH (1 mL) is added dimethylamine (2 M, 20 eq.). The reaction mixture is stirred at 70° C. overnight. TLC indicates the extent of the reaction and the mixture is concentrated under reduced pressure to give a residue. The residue is purified by column chromatography to give Compound 373.

To a solution of this residue in DCM (1 mL) is added HCl/dioxane (1.5 mL, 1 M) at 25° C. The reaction mixture is stirred at 25° C. for 1 hour. The reaction mixture is concentrated and lyophilized to give Compound 373 in HCl salt form.

### General Procedure for Preparation of Compound 374

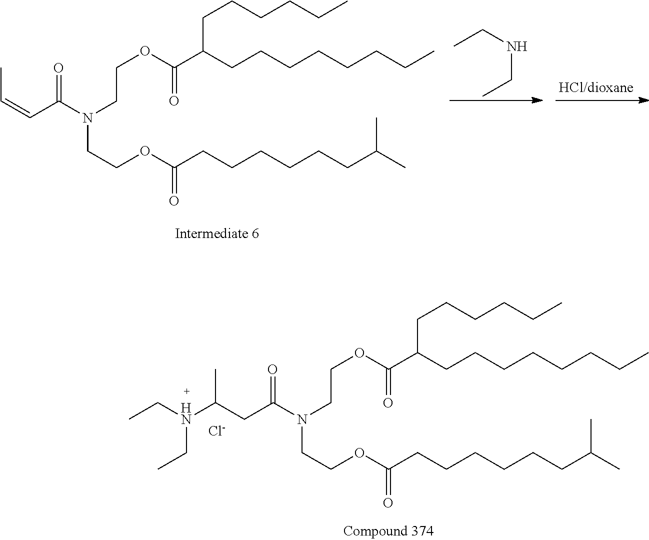

To a solution of Intermediate 6 (1 eq.) in THF (1 mL) and MeOH (1 mL) is added diethylamine (2 M, 20 eq.). The reaction mixture is stirred at 70° C. overnight. TLC indicates the extent of the reaction and the mixture is concentrated under reduced pressure to give a residue. The residue is purified by column chromatography to give Compound 374.

To a solution of this residue in DCM (1 mL) is added HCl/dioxane (1.5 mL, 1 M) at 25° C. The reaction mixture is stirred at 25° C. for 1 hour. The reaction mixture is concentrated and lyophilized to give Compound 374 in HCl salt form.

### Example 2. Synthesis and Characterization of Series 200 Lipidoids

Procedures of Example 1 were used to access Series 200 lipidoids, except that D-amino acid starting materials were used in place of the L-amino acid starting materials.

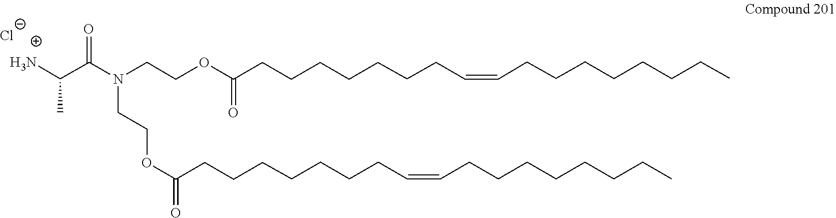 [SMILES: [Cl-] ; CCCCCCCC/C=C\CCCCCCCC(=O)OCCN(CCOC(=O)CCCCCCC/C=C\CCCCCCCC)C(=O)[C@H](C)[NH3+]]

LCMS [M+1]⁺=705.5. ¹H NMR (400 MHz, chloroform-d) δ=8.50 (br s, 3H), 5.42-5.29 (m, 4H), 4.66 (br d, J=6.0 Hz, 1H), 4.40-4.25 (m, 2H), 4.21 (br t, J=5.2 Hz, 2H), 3.94-3.84 (m, 1H), 3.67 (br s, 2H), 3.48-3.38 (m, 1H), 2.36-2.26 (m, 4H), 2.06-1.96 (m, 8H), 1.62 (br d, J=6.4 Hz, 7H), 1.29 (br d, J=11.6 Hz, 40H), 0.93-0.83 (m, 6H).

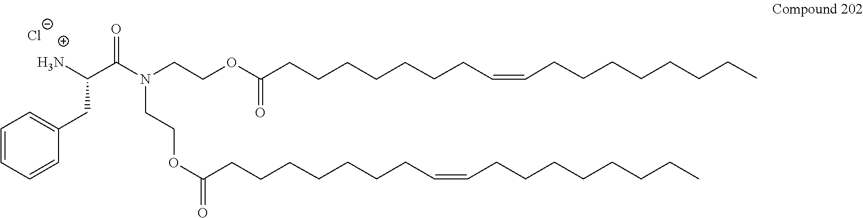 [SMILES: CCCCCCCC/C=C\CCCCCCCC(=O)OCCN(CCOC(=O)CCCCCCC/C=C\CCCCCCCC)C(=O)[C@@H]([NH3+])Cc1ccccc1 ; [Cl-]]

LCMS [M+1]+=781.6. ¹H NMR (400 MHz, chloroform-d) δ=7.32-7.27 (m, 2H), 7.26-7.17 (m, 3H), 5.51-5.24 (m, 4H), 4.25-3.99 (m, 4H), 3.94 (t, J=7.2 Hz, 1H), 3.80 (td, J=5.6, 14.1 Hz, 1H), 3.48-3.20 (m, 3H), 2.98 (dd, J=7.2, 13.1 Hz, 1H), 2.82 (dd, J=6.8, 13.2 Hz, 1H), 2.28 (q, J=8.0 Hz, 4H), 2.01 (q, J=6.4 Hz, 8H), 1.64-1.50 (m, 4H), 1.41-1.19 (m, 40H), 0.96-0.82 (m, 6H).

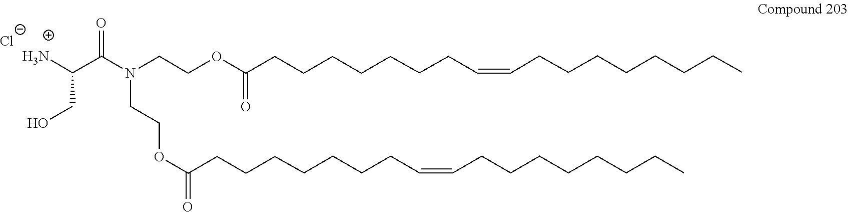 [SMILES: [Cl-] ; CCCCCCCC/C=C\CCCCCCCC(=O)OCCN(CCOC(=O)CCCCCCC/C=C\CCCCCCCC)C(=O)[C@@H]([NH3+])CO]

LCMS [M+1]⁺=721.6. ¹H NMR (400 MHz, methanol-d₄) δ=5.35 (t, J=4.8 Hz, 4H), 4.44 (dd, J=4.4, 7.5 Hz, 1H), 4.26 (qd, J=5.6, 14.8 Hz, 4H), 4.00-3.78 (m, 3H), 3.77-3.60 (m, 2H), 3.58-3.45 (m, 1H), 2.45-2.28 (m, 4H), 2.03 (br d, J=4.4 Hz, 8H), 1.61 (br d, J=6.4 Hz, 4H), 1.32 (br d, J=13.2 Hz, 40H), 0.90 (br t, J=6.8 Hz, 6H).

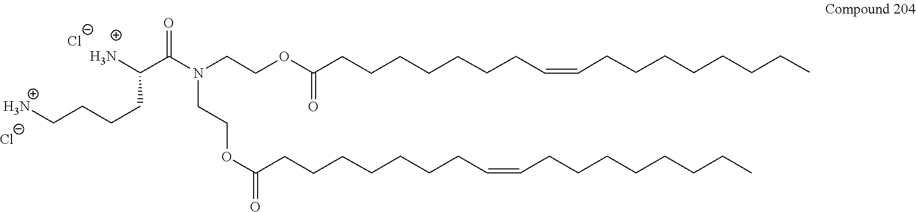 [SMILES: [Cl-] ; CCCCCCCC/C=C\CCCCCCCC(=O)OCCN(CCOC(=O)CCCCCCC/C=C\CCCCCCCC)C(=O)[C@@H]([NH3+])CCCC[NH3+] ; [Cl-]]

LCMS [M+1]⁺=762.7. ¹H NMR (400 MHz, DMSO-d₆) δ=8.34-7.98 (m, 5H), 5.46-5.23 (m, 4H), 4.34-4.05 (m, 5H), 3.83-3.67 (m, 2H), 3.59-3.51 (m, 1H), 3.40 (br dd, J=6.0, 8.3 Hz, 1H), 2.73 (br t, J=6.8 Hz, 2H), 2.36-2.22 (m, 4H), 1.98 (br d, J=4.4 Hz, 8H), 1.69 (br s, 2H), 1.50 (br s, 8H), 1.25 (br d, J=2.0 Hz, 40H), 0.94-0.78 (m, 6H).

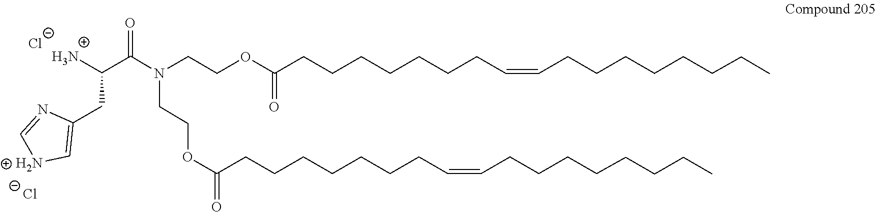 [SMILES: CCCCCCCC/C=C\CCCCCCCC(=O)OCCN(CCOC(=O)CCCCCCC/C=C\CCCCCCCC)C(=O)[C@@H]([NH3+])CC1=C[NH2+]C=N1 ; [Cl-] ; [Cl-]]

LCMS [M+1]⁺=771.7. ¹H NMR (400 MHz, DMSO-d₆) δ=15.29-13.46 (m, 2H), 8.65-7.96 (m, 4H), 7.44-7.22 (m, 1H), 5.41-5.23 (m, 4H), 4.64-4.47 (m, 1H), 4.21 (br dd, J=4.8, 6.0 Hz, 1H), 4.17-4.05 (m, 3H), 3.89-3.73 (m, 1H), 3.93-3.72 (m, 1H), 3.66-3.45 (m, 4H), 2.37-2.17 (m, 4H), 1.97 (br d, J=4.8 Hz, 8H), 1.49 (br s, 4H), 1.36-1.15 (m, 40H), 0.88-0.87 (m, 1H), 0.85 (br t, J=6.4 Hz, 6H).

### Example 3. Synthesis and Characterization of Series 400 Lipidoids

### Representative Synthesis.

Headgroup=L-phenylalanine

### Synthesis of Compound 3:

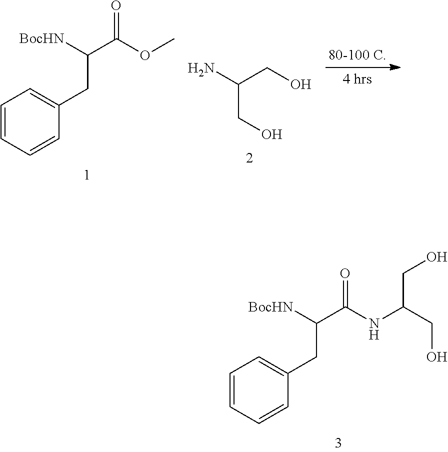

A mixture of Compound 2 (3.26 g, 35.80 mmol, 2 eq.) and Compound 1 (5 g, 17.90 mmol, 1 eq.) was heated to 100° C. for 3 hours. TLC (Dichloromethane:Methanol=10:1, R_{f}=0.5) indicated the reaction was consumed completely and one new spot formed. The residue was diluted with saturated NaHCO₃ solution (20 mL) and extracted with DCM (50 mL*3), dried over Na₂SO₄, filtered and concentrated under reduced pressure to give a residue. The residue was purified by column chromatography (SiO₂, DCM:MeOH=100:1 to 9:1) to give Compound 3 (10 g, 27.78 mmol, 77.59% yield, 94% purity) was obtained as a white solid.

Compound 3: LCMS [M+1]⁺=339.2, LCMS [M+23]⁺=361.2. ¹H NMR (400 MHz, chloroform-d) δ=7.38-7.28 (m, 3H), 7.26-7.22 (m, 2H), 6.39 (br d, J=6.8 Hz, 1H), 5.08 (br s, 1H), 3.85-3.69 (m, 4H), 3.69-3.62 (m, 1H), 3.15-3.01 (m, 2H), 2.71 (br s, 1H), 2.33 (br d, J=4.4 Hz, 1H), 1.61 (s, 1H), 1.43 (s, 9H).

### Representative Synthesis of Compound 5:

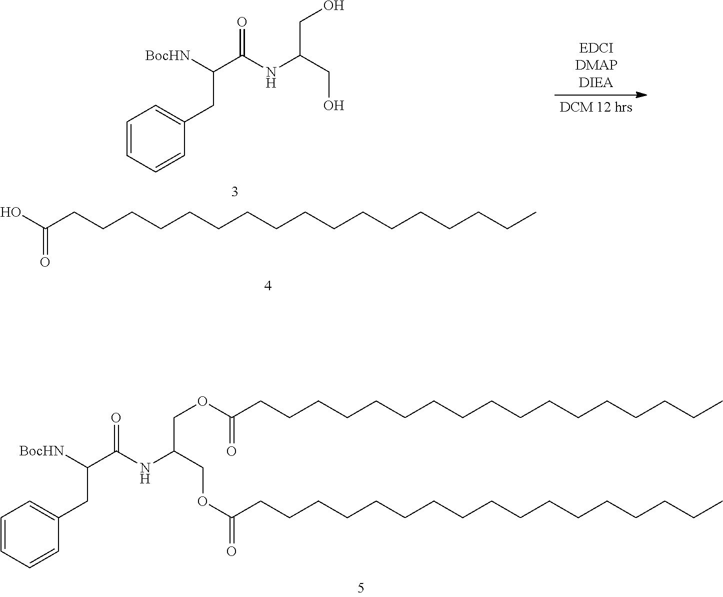

To a solution of Compound 4 (840.66 mg, 2.96 mmol, 994.86 μL, 2 eq.) in DCM (8 mL) was added Compound 3 (500.00 mg, 1.48 mmol, 1 eq.), DMAP (90.25 mg, 738.78 μmol, 0.5 eq.), EDCI (708.12 mg, 3.69 mmol, 2.5 eq.) and DIEA (572.89 mg, 4.43 mmol, 772.09 μL, 3 eq.), and then the mixture was stirred at 20° C. for 16 hours under N₂ atmosphere. LC-MS showed Compound 3 was consumed completely and one main peak with desired MS was detected. The residue was quenched by saturated NaHCO₃ solution (20 mL) and extracted with DCM (20 mL*2). The combined organic layers were dried over Na₂SO₄, filtered and concentrated under reduced pressure to give a residue. The residue was purified by column chromatography (SiO₂, Petroleum ether:Ethyl acetate=25:1 to 9:1) to give Compound 5 (0.3 g, 344.31 μmol, 23.30% yield) as a white solid.

Compound 5: LCMS [M−100+1]+=771.6. ¹H NMR (400 MHz, chloroform-d) δ=7.35-7.28 (m, 2H), 7.27-7.16 (m, 3H), 5.99 (br d, J=8.4 Hz, 1H), 5.04 (br s, 1H), 4.40-4.33 (m, 1H), 4.30 (br d, J=6.8 Hz, 1H), 4.12 (dd, J=5.2, 11.3 Hz, 1H), 4.06 (br dd, J=4.8, 11.4 Hz, 1H), 3.98 (dd, J=5.6, 11.3 Hz, 1H), 3.82 (br dd, J=4.4, 10.8 Hz, 1H), 3.14-3.06 (m, 1H), 3.03-2.95 (m, 1H), 2.26 (dt, J=3.2, 7.6 Hz, 4H), 1.58 (br d, J=6.8 Hz, 4H), 1.43 (s, 9H), 1.26 (s, 56H), 0.89 (t, J=6.8 Hz, 6H).

### Representative Synthesis of Series 400 Lipid:

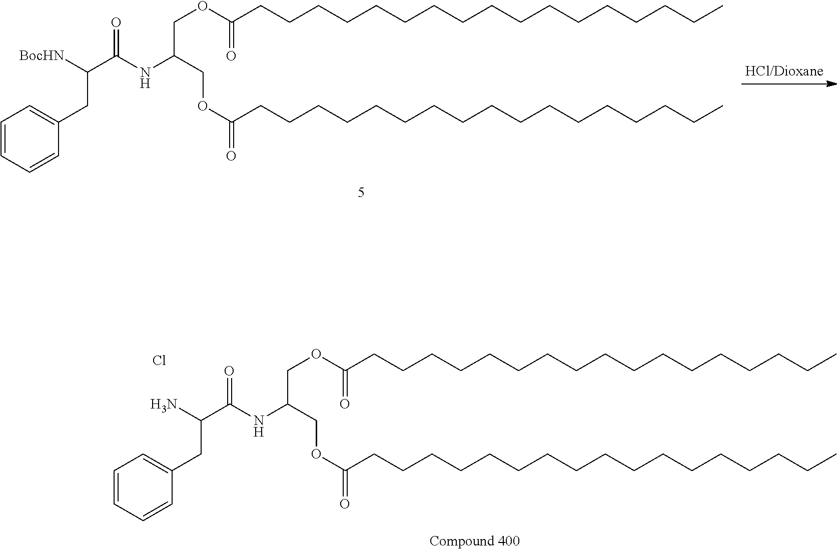 [SMILES: CCCCCCCCCCCCCCCCCC(=O)OCC(COC(=O)CCCCCCCCCCCCCCCCC)NC(=O)C(Cc1ccccc1)NC(=O)OC(C)(C)C ; CCCCCCCCCCCCCCCCCC(=O)OCC(COC(=O)CCCCCCCCCCCCCCCCC)NC(=O)C(N)Cc1ccccc1 ; Cl]

A mixture of Compound 5 (270 mg, 309.88 μmol, 1 eq) in HCl/dioxane (3 mL, 4M) was was stirred at 20° C. for 4 hours under N₂ atmosphere. TLC (Petroleum ether:Ethyl acetate=4:1, R_{f}=0.52) indicated Compound 5 was consumed completely and one major new spot was detected. The reaction mixture was concentrated under reduced pressure to give crude product. The crude product was purified by re-crystallization from DMSO (2 mL) at 45° C., then the mixture was filtered, and the filter cake was lyophilized to give Compound 400 (130 mg, 160.96 μmol, 51.94% yield, HCl) as white solid.

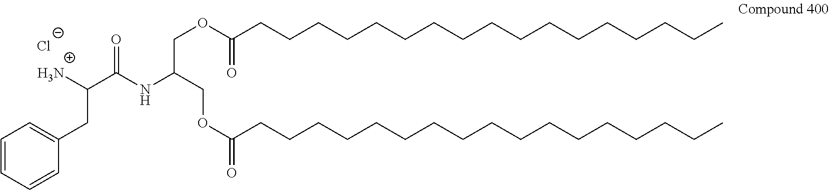 [SMILES: CCCCCCCCCCCCCCCCCC(=O)OCC(COC(=O)CCCCCCCCCCCCCCCCC)NC(=O)C([NH3+])Cc1ccccc1 ; [Cl-]]

LCMS [M+1]+=771.6. ¹H NMR (400 MHz, DMSO-d₆) δ=8.54 (br d, J=6.4 Hz, 1H), 8.17 (br s, 3H), 7.46-7.11 (m, 5H), 4.26-4.13 (m, 1H), 4.11-3.85 (m, 5H), 3.00 (br s, 2H), 2.35-2.21 (m, 4H), 1.50 (br s, 4H), 1.23 (br s, 56H), 0.85 (br s, 6H).

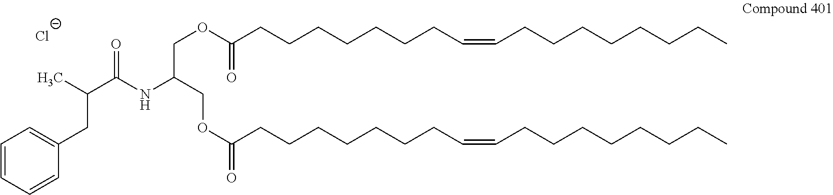 [SMILES: CCCCCCCC/C=C\CCCCCCCC(=O)OCC(COC(=O)CCCCCCC/C=C\CCCCCCCC)NC(=O)C(C)Cc1ccccc1 ; [Cl-]]

LCMS [M+1]+=767.7. ¹H NMR (400 MHz, DMSO-d₆) δ=8.60 (br d, J=8.0 Hz, 1H), 8.42-7.91 (m, 3H), 7.43-7.08 (m, 5H), 5.31 (br d, J=4.4 Hz, 4H), 4.25-4.15 (m, 1H), 4.10-4.04 (m, 1H), 4.04-3.96 (m, 1H), 3.96-3.95 (m, 1H), 3.92 (br d, J=5.2 Hz, 2H), 3.00 (br t, J=7.6 Hz, 2H), 2.28 (td, J=7.6, 15.2 Hz, 4H), 1.97 (br s, 8H), 1.50 (br d, J=5.6 Hz, 4H), 1.24 (br s, 40H), 0.85 (br t, J=6.4 Hz, 6H).

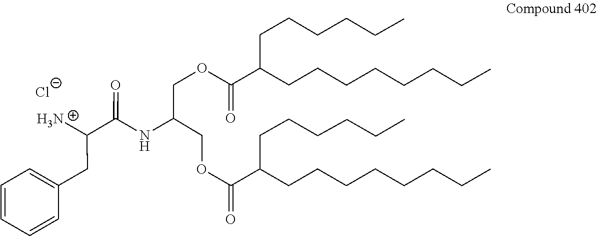 [SMILES: CCCCCCCCC(CCCCCC)C(=O)OCC(COC(=O)C(CCCCCC)CCCCCCCC)NC(=O)C([NH3+])Cc1ccccc1 ; [Cl-]]

LCMS [M+1]⁺=715.6. ¹H NMR (400 MHz, DMSO-d₆) δ=8.71 (br d, J=7.6 Hz, 1H), 8.19-8.07 (m, 2H), 7.38-7.17 (m, 5H), 4.25-4.07 (m, 2H), 4.02-3.85 (m, 4H), 3.10-3.03 (m, 1H), 3.13-3.01 (m, 1H), 3.01-2.92 (m, 1H), 2.35-2.26 (m, 2H), 1.55-1.36 (m, 8H), 1.28-1.11 (m, 42H), 0.87-0.77 (m, 12H).

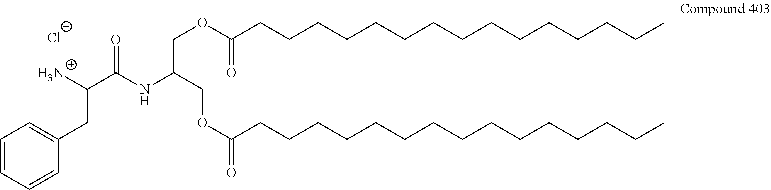 [SMILES: CCCCCCCCCCCCCCCC(=O)OCC(COC(=O)CCCCCCCCCCCCCCC)NC(=O)C([NH3+])Cc1ccccc1 ; [Cl-]]

LCMS [M+1]⁺=705.5. ¹H NMR (400 MHz, DMSO-d₆) δ=8.63-8.46 (m, 1H), 8.28-7.55 (m, 3H), 7.36-7.20 (m, 5H), 4.25-4.16 (m, 1H), 4.10-3.94 (m, 3H), 3.93 (br d, J=5.6 Hz, 2H), 3.06-2.91 (m, 2H), 2.32-2.22 (m, 4H), 1.56-1.44 (m, 4H), 1.32-1.14 (m, 48H), 0.94-0.76 (m, 6H).

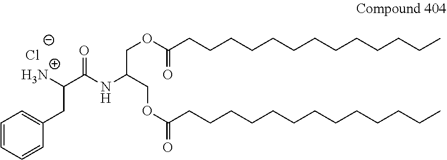 [SMILES: CCCCCCCCCCCCCC(=O)OCC(COC(=O)CCCCCCCCCCCCC)NC(=O)C([NH3+])Cc1ccccc1 ; [Cl-]]

LCMS [M+1]⁺=659.5. ¹HNMR (400 MHz, DMSO-d₆) δ=8.64-8.54 (m, 1H), 8.31-7.69 (m, 3H), 7.38-7.14 (m, 5H), 4.25-4.15 (m, 1H), 4.11-3.89 (m, 5H), 3.08-2.92 (m, 2H), 2.33-2.21 (m, 4H), 1.55-1.44 (m, 4H), 1.35-1.13 (m, 40H), 0.85 (br t, J=6.4 Hz, 6H).

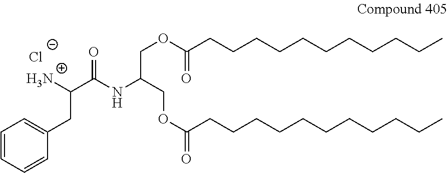 [SMILES: CCCCCCCCCCCC(=O)OCC(COC(=O)CCCCCCCCCCC)NC(=O)C([NH3+])Cc1ccccc1 ; [Cl-]]

LCMS [M+1]⁺=603.5. ¹H NMR (400 MHz, DMSO-d₆) δ=8.60 (br d, J=8.0 Hz, 1H), 8.14 (br d, J=3.2 Hz, 3H), 7.41-7.18 (m, 5H), 4.26-4.14 (m, 1H), 4.12-3.90 (m, 5H), 3.09-2.92 (m, 2H), 2.28 (td, J=7.6, 15.0 Hz, 4H), 1.56-1.44 (m, 4H), 1.30-1.16 (m, 32H), 0.85 (t, J=6.8 Hz, 6H).

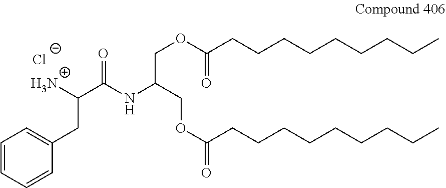 [SMILES: CCCCCCCCCC(=O)OCC(COC(=O)CCCCCCCCC)NC(=O)C([NH3+])Cc1ccccc1 ; [Cl-]]

LCMS [M+1]⁺=547.5. ¹H NMR (400 MHz, DMSO-d₆) δ=8.63 (br d, J=6.8 Hz, 1H), 8.11 (br d, J=1.6 Hz, 3H), 7.38-7.15 (m, 5H), 4.29-4.14 (m, 1H), 4.10-3.88 (m, 5H), 3.10-2.92 (m, 2H), 2.28 (td, J=7.6, 15.2 Hz, 4H), 1.58-1.42 (m, 4H), 1.22 (br d, J=11.6 Hz, 24H), 0.96-0.72 (m, 6H).

### Example 4. Synthesis and Characterization of Series 600 Lipidoids

The same synthetic route of Series 100 compounds was followed for the Series 600 compounds. Lipid precursors were prepared according to Small Sci. 2023, 3, 2200071.

### General Synthetic Overview of Series 600 Compounds

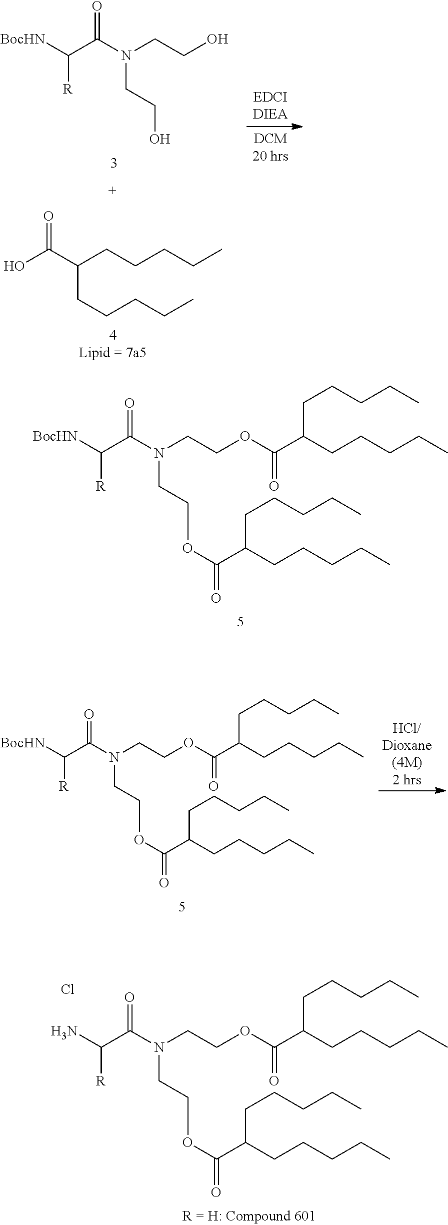

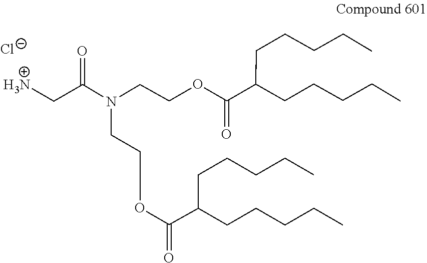 [SMILES: [Cl-] ; CCCCCC(CCCCC)C(=O)OCCN(CCOC(=O)C(CCCCC)CCCCC)C(=O)C[NH3+]]

LCMS [M+1]⁺=527.4. ¹H NMR (400 MHz, DMSO-d₆) δ=8.05-7.74 (m, 3H), 4.22-4.08 (m, 4H), 3.90-3.80 (m, 2H), 3.65-3.54 (m, 4H), 2.37-2.27 (m, 2H), 1.51-1.37 (m, 8H), 1.29-1.16 (m, 24H), 0.84 (br t, J=6.8 Hz, 12H).

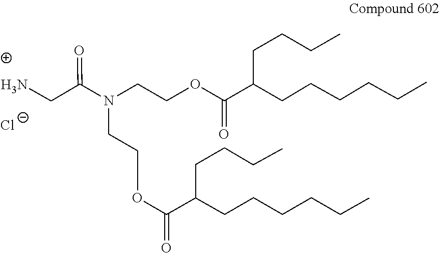 [SMILES: [Cl-] ; CCCCCCC(CCCC)C(=O)OCCN(CCOC(=O)C(CCCC)CCCCCC)C(=O)CN]

LCMS [M+1]⁺=527.5. ¹H NMR (400 MHz, DMSO-d₆) δ=7.98-7.68 (m, 3H), 4.24-4.05 (m, 4H), 3.93-3.80 (m, 2H), 3.67-3.49 (m, 4H), 2.39-2.25 (m, 2H), 1.53-1.37 (m, 8H), 1.30-1.13 (m, 24H), 0.96-0.75 (m, 12H).

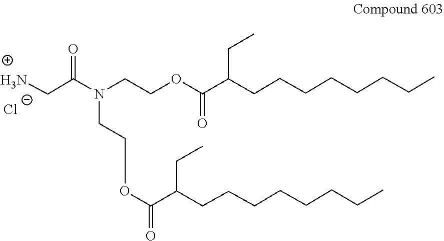 [SMILES: [Cl-] ; CCCCCCCCC(CC)C(=O)OCCN(CCOC(=O)C(CC)CCCCCCCC)C(=O)C[NH3+]]

LCMS [M+1]⁺=527.5. ¹H NMR (400 MHz, DMSO-d₆) δ=7.70-8.11 (m, 3H), 4.16-4.23 (m, 2H), 4.10-4.16 (m, 2H), 3.83-3.90 (m, 2H), 3.55-3.63 (m, 4H), 2.20-2.34 (m, 2H), 1.38-1.53 (m, 8H), 1.23 (s, 24H), 0.78-0.89 (m, 12H).

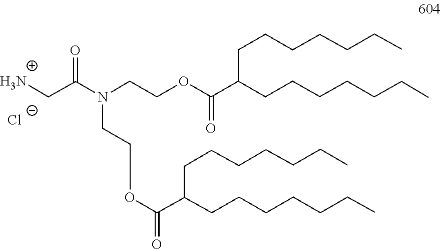 [SMILES: [Cl-] ; CCCCCCCC(CCCCCCC)C(=O)OCCN(CCOC(=O)C(CCCCCCC)CCCCCCC)C(=O)C[NH3+]]

LCMS [M+1]⁺=639.6. ¹H NMR (400 MHz, DMSO-d₆) δ=8.20-7.42 (m, 3H), 4.21-4.15 (m, 2H), 4.14-4.08 (m, 2H), 3.90-3.83 (m, 2H), 3.63-3.53 (m, 4H), 2.35-2.25 (m, 2H), 1.51-1.37 (m, 8H), 1.23 (s, 40H), 0.85 (t, J=6.8 Hz, 12H).

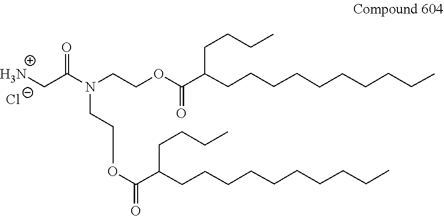 [SMILES: [Cl-] ; CCCCCCCCCCC(CCCC)C(=O)OCCN(CCOC(=O)C(CCCC)CCCCCCCCCC)C(=O)C[NH3+]]

LCMS [M+1]⁺=639.6. ¹H NMR (400 MHz, DMSO-d₆) δ=8.14-7.61 (m, 3H), 4.23-4.15 (m, 2H), 4.14-4.07 (m, 2H), 3.92-3.81 (m, 2H), 3.67-3.52 (m, 4H), 2.36-2.24 (m, 2H), 1.54-1.37 (m, 8H), 1.23 (s, 40H), 0.93-0.77 (m, 12H).

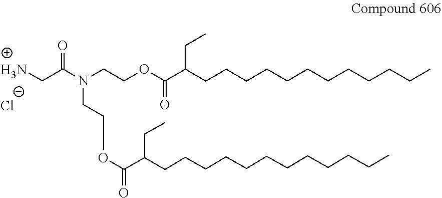 [SMILES: Cl ; CCCCCCCCCCCCC(CC)C(=O)OCCN(CCOC(=O)C(CC)CCCCCCCCCCCC)C(=O)CN]

LCMS [M+1]⁺=639.6. ¹H NMR (400 MHz, DMSO-d₆) δ=7.93-7.70 (m, 3H), 4.28-4.00 (m, 4H), 3.94-3.78 (m, 2H), 3.68-3.51 (m, 4H), 2.34-2.18 (m, 2H), 1.58-1.37 (m, 9H), 1.35-1.06 (m, 40H), 0.98-0.71 (m, 12H).

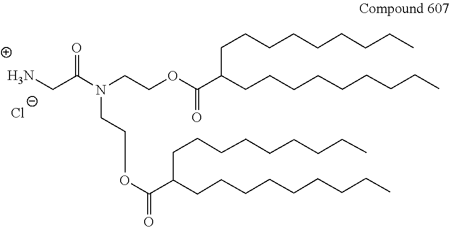 [SMILES: [Cl-] ; CCCCCCCCCC(CCCCCCCCC)C(=O)OCCN(CCOC(=O)C(CCCCCCCCC)CCCCCCCCC)C(=O)CN]

LCMS [M+1]⁺=751.7. ¹H NMR (400 MHz, DMSO-d₆) δ=8.17-7.30 (m, 3H), 4.23-4.05 (m, 4H), 3.91-3.79 (m, 2H), 3.65-3.52 (m, 4H), 2.33-2.28 (m, 2H), 1.49-1.38 (m, 8H), 1.23 (s, 56H), 0.91-0.77 (m, 12H).

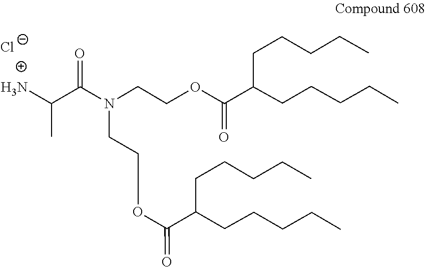 [SMILES: [Cl-] ; CCCCCC(CCCCC)C(=O)OCCN(CCOC(=O)C(CCCCC)CCCCC)C(=O)C(C)[NH3+]]

LCMS [M+1]⁺=541.5. ¹H NMR (400 MHz, DMSO-d₆) δ=8.25-7.14 (m, 3H), 4.27-4.03 (m, 5H), 3.82-3.72 (m, 1H), 3.71-3.55 (m, 2H), 3.42 (m, 1H), 2.36-2.25 (m, 2H), 1.52-1.38 (m, 8H), 1.28 (br d, J=6.8 Hz, 3H), 1.22 (br s, 24H), 0.84 (br t, J=6.8 Hz, 12H).

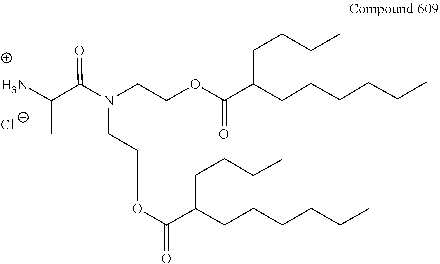 [SMILES: [Cl-] ; CCCCCCC(CCCC)C(=O)OCCN(CCOC(=O)C(CCCC)CCCCCC)C(=O)C(C)N]

LCMS [M+1]⁺=785.5. ¹H NMR (400 MHz, DMSO-d6) δ=8.31-7.81 (m, 3H), 4.29 (q, J=6.4 Hz, 1H), 4.23-4.02 (m, 4H), 3.85-3.74 (m, 1H), 3.73-3.52 (m, 2H), 3.46-3.36 (m, 1H), 2.37-2.24 (m, 2H), 1.55-1.38 (m, 8H), 1.32 (br d, J=6.8 Hz, 3H), 1.22 (br s, 24H), 0.97-0.75 (m, 12H).

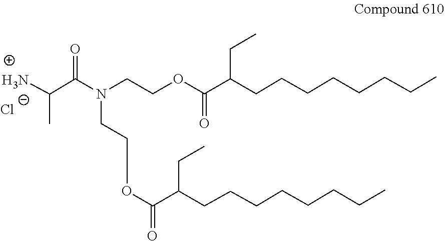 [SMILES: [Cl-] ; CCCCCCCCC(CC)C(=O)OCCN(CCOC(=O)C(CC)CCCCCCCC)C(=O)C(C)[NH3+]]

LCMS [M+1]⁺=541.5. ¹H NMR (400 MHz, DMSO-d₆) δ=7.95 (br s, 3H), 4.29-4.13 (m, 4H), 4.12-4.04 (m, 1H), 3.86-3.75 (m, 1H), 3.73-3.53 (m, 2H), 3.40 (m, 2H), 2.32-2.17 (m, 2H), 1.56-1.37 (m, 8H), 1.30 (br d, J=6.4 Hz, 3H), 1.28-1.09 (m, 24H), 0.95-0.72 (m, 12H).

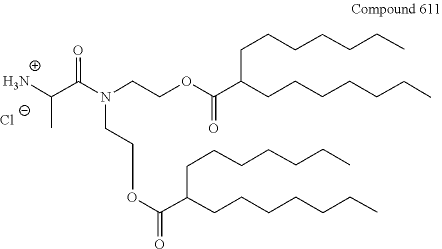 [SMILES: [Cl-] ; CCCCCCCC(CCCCCCC)C(=O)OCCN(CCOC(=O)C(CCCCCCC)CCCCCCC)C(=O)C(C)[NH3+]]

LCMS [M+1]⁺=653.6. ¹H NMR (400 MHz, DMSO-d₆) δ=7.78 (br d, J=1.6 Hz, 3H), 4.30-4.10 (m, 4H), 4.10-3.99 (m, 1H), 3.85-3.72 (m, 1H), 3.71-3.51 (m, 2H), 3.45-3.38 (m, 1H), 2.41-2.19 (m, 2H), 1.58-1.45 (m, 4H), 1.44-1.34 (m, 4H), 1.29 (br d, J=6.8 Hz, 3H), 1.28-1.10 (m, 40H), 0.85 (t, J=6.8 Hz, 12H).

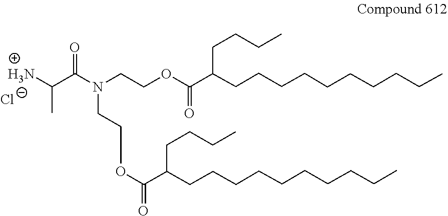 [SMILES: [Cl-] ; CCCCCCCCCCC(CCCC)C(=O)OCCN(CCOC(=O)C(CCCC)CCCCCCCCCC)C(=O)C(C)[NH3+]]

LCMS [M+1]⁺=653.6. ¹H NMR (400 MHz, DMSO-d₆) δ=8.27-7.82 (m, 3H), 4.33-4.25 (m, 1H), 4.24-4.12 (m, 3H), 4.11-4.04 (m, 1H), 3.83-3.74 (m, 1H), 3.72-3.53 (m, 2H), 3.45-3.37 (m, 1H), 2.25 (s, 1H), 1.55-1.45 (m, 4H), 1.45-1.37 (m, 4H), 1.34-1.30 (m, 3H), 1.29-1.13 (m, 40H), 0.92-0.78 (m, 12H).

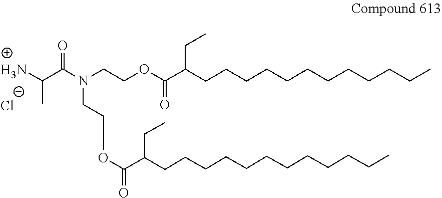 [SMILES: [Cl-] ; CCCCCCCCCCCCC(CC)C(=O)OCCN(CCOC(=O)C(CC)CCCCCCCCCCCC)C(=O)C(C)[NH3+]]

LCMS [M+1]⁺=653.6. ¹H NMR (400 MHz, DMSO-d6) δ=8.36-7.63 (m, 3H), 4.32-4.02 (m, 5H), 3.84-3.75 (m, 1H), 3.73-3.53 (m, 2H), 3.44-3.37 (m, 1H), 2.31-2.16 (m, 2H), 1.49 (br d, J=4.8 Hz, 4H), 1.42 (m, 4H), 1.30 (br d, J=6.8 Hz, 3H), 1.23 (br s, 40H), 0.92-0.74 (m, 12H).

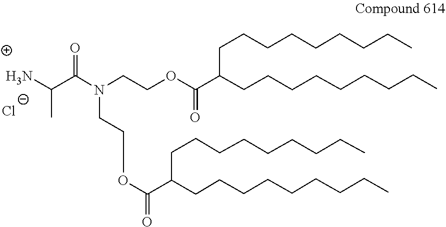 [SMILES: [Cl-] ; CCCCCCCCCC(CCCCCCCCC)C(=O)OCCN(CCOC(=O)C(CCCCCCCCC)CCCCCCCCC)C(=O)C(C)N]

LCMS [M+1]⁺=765.7. ¹H NMR (400 MHz, DMSO-d6) δ=8.42-7.62 (m, 3H), 4.34-4.03 (m, 5H), 3.84-3.73 (m, 1H), 3.72-3.53 (m, 2H), 3.47-3.37 (m, 1H), 2.38-2.23 (m, 2H), 1.55-1.38 (m, 8H), 1.35-1.10 (m, 59H), 0.86 (br t, J=6.4 Hz, 12H).

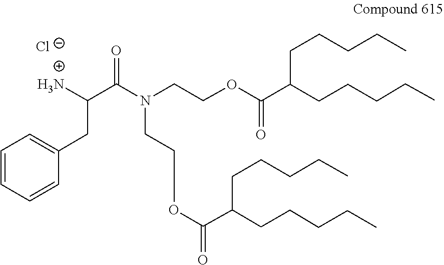 [SMILES: CCCCCC(CCCCC)C(=O)OCCN(CCOC(=O)C(CCCCC)CCCCC)C(=O)C([NH3+])Cc1ccccc1 ; [Cl-]]

LCMS [M+1]⁺=617.5. ¹H NMR (400 MHz, chloroform-d) δ=8.78-8.40 (m, 3H), 7.35-7.27 (m, 5H), 4.86-4.74 (m, 1H), 4.29-3.97 (m, 4H), 3.93-3.82 (m, 1H), 3.56-3.47 (m, 1H), 3.35-3.17 (m, 3H), 3.13-2.99 (m, 1H), 2.36-2.24 (m, 2H), 1.63-1.49 (m, 4H), 1.48-1.36 (m, 4H), 1.26 (br s, 24H), 0.94-0.82 (m, 12H).

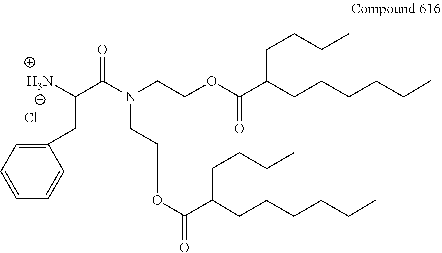 [SMILES: CCCCCCC(CCCC)C(=O)OCCN(CCOC(=O)C(CCCC)CCCCCC)C(=O)C([NH3+])Cc1ccccc1 ; [Cl-]]

LCMS [M+1]⁺=617.5. ¹H NMR (400 MHz, DMSO-d₆) δ=8.40-7.69 (m, 3H), 7.33-7.20 (m, 5H), 4.48 (br t, J=6.8 Hz, 1H), 4.13-3.96 (m, 4H), 3.81-3.72 (m, 1H), 3.40-3.33 (m, 2H), 3.31-3.22 (m, 2H), 3.01 (br d, J=6.8 Hz, 2H), 2.32-2.22 (m, 2H), 1.54-1.37 (m, 8H), 1.21 (br s, 24H), 0.88-0.79 (m, 12H).

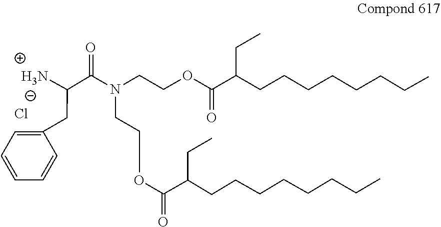 [SMILES: CCCCCCCCC(CC)C(=O)OCCN(CCOC(=O)C(CC)CCCCCCCC)C(=O)C([NH3+])Cc1ccccc1 ; [Cl-]]

LCMS [M+1]⁺=617.5. ¹H NMR (400 MHz, chloroform-d) δ=8.51-8.12 (m, 3H), 7.30 (m, 4H), 7.26-7.18 (m, 1H), 4.91-4.68 (m, 1H), 4.27-3.94 (m, 4H), 3.93-3.83 (m, 1H), 3.54-3.44 (m, 1H), 3.37-3.15 (m, 3H), 3.14-3.04 (m, 1H), 2.26-2.18 (m, 2H), 1.64-1.38 (m, 8H), 1.33-1.21 (m, 24H), 0.92-0.81 (m, 12H)

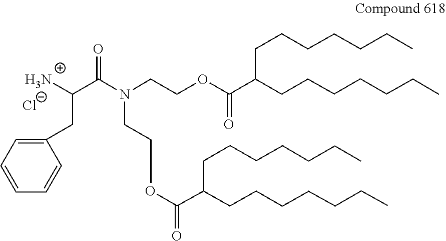 [SMILES: CCCCCCCC(CCCCCCC)C(=O)OCCN(CCOC(=O)C(CCCCCCC)CCCCCCC)C(=O)C([NH3+])Cc1ccccc1 ; [Cl-]]

LCMS [M+1]⁺=729.6 LCMS [2M+1]⁺=1459.2. ¹H NMR (400 MHz, DMSO-d₆) δ=8.51-7.70 (m, 3H), 7.31-7.20 (m, 5H), 4.55-4.47 (m, 1H), 4.12-3.98 (m, 4H), 3.82-3.71 (m, 1H), 3.37-3.34 (m, 1H), 3.30-3.22 (m, 2H), 3.06-2.96 (m, 2H), 2.31-2.23 (m, 2H), 1.49-1.35 (m, 8H), 1.25-1.13 (m, 40H), 0.88-0.81 (m, 12H).

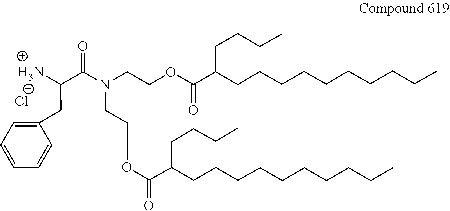 [SMILES: CCCCCCCCCCC(CCCC)C(=O)OCCN(CCOC(=O)C(CCCC)CCCCCCCCCC)C(=O)C([NH3+])Cc1ccccc1 ; [Cl-]]

LCMS [M+1]⁺=729.6. ¹H NMR (400 MHz, DMSO-d₆) δ=8.40-7.94 (m, 3H), 7.32-7.20 (m, 5H), 4.55-4.44 (m, 1H), 4.12-3.96 (m, 4H), 3.81-3.72 (m, 1H), 3.38-3.33 (m, 1H), 3.22 (br s, 2H), 3.08-2.94 (m, 2H), 2.32-2.23 (m, 2H), 1.51-1.37 (m, 8H), 1.28-1.14 (m, 40H), 0.89-0.80 (m, 12H).

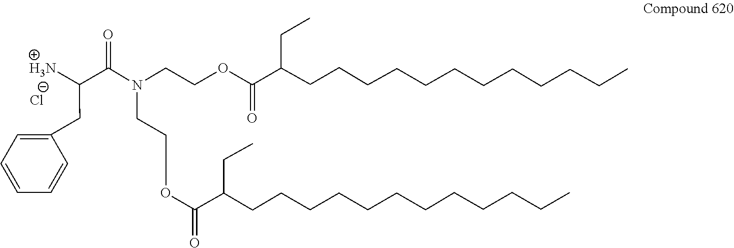 [SMILES: CCCCCCCCCCCCC(CC)C(=O)OCCN(CCOC(=O)C(CC)CCCCCCCCCCCC)C(=O)C([NH3+])Cc1ccccc1 ; [Cl-]]

LCMS [M−100+1]+=729.5. ¹H NMR (400 MHz, DMSO-d₆) δ=7.32-7.15 (m, 5H), 7.13-7.02 (m, 1H), 4.63-4.51 (m, 1H), 4.20-3.98 (m, 4H), 3.74-3.49 (m, 3H), 3.47-3.36 (m, 1H), 2.88-2.67 (m, 2H), 2.26-2.13 (m, 2H), 1.53-1.34 (m, 9H), 1.29 (s, 48H), 0.88-0.75 (m, 12H).

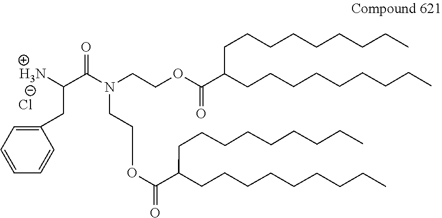 [SMILES: CCCCCCCCCC(CCCCCCCCC)C(=O)OCCN(CCOC(=O)C(CCCCCCCCC)CCCCCCCCC)C(=O)C([NH3+])Cc1ccccc1 ; [Cl-]]

LCMS [M+1]⁺=841.7. ¹H NMR (400 MHz, DMSO-d₆) δ=8.42-7.47 (m, 3H), 7.31-7.20 (m, 5H), 4.50-4.43 (m, 1H), 4.11-3.97 (m, 4H), 3.79-3.72 (m, 1H), 3.40-3.35 (m, 1H), 3.30-3.22 (m, 2H), 3.03-2.95 (m, 2H), 2.30-2.21 (m, 2H), 1.50-1.37 (m, 8H), 1.26-1.15 (m, 56H), 0.89-0.81 (m, 12H).

### Example 5. Synthesis and Characterization of Series 800 Lipidoids

For non-commercially available materials Step 1 is followed. For commercially available amino methyl esters, Step 1 is not required.

### Step 1: Methyl Ester Synthesis:

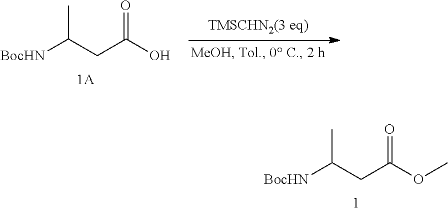

To a solution of Compound 1A (2 g, 9.84 mmol, 1 eq.) in MeOH (20 mL) and Tol. (70 mL) was added TMSCHN₂ (2 M, 14.76 mL, 3 eq) at 0° C. The reaction mixture was stirred at 0° C. for 2 hours. TLC (PE:EtOAc=3:1, R_{f}=0.8) indicated Compound 1A was consumed completely and one new spot formed. The reaction mixture was concentrated under reduced pressure to give a residue. The residue was purified by column chromatography (SiO₂, PE:EtOAc=10:1) to give Compound 1 (2.1 g, 9.67 mmol, 98.22% yield) as colorless oil. ¹H NMR (400 MHz, chloroform-d) δ=4.92 (s, 1H), 4.04 (br s, 1H), 3.69 (s, 3H), 2.61-2.42 (m, 2H), 1.44 (s, 9H), 1.22 (d, J=6.8 Hz, 3H).

### Step 2: Headgroup Coupling:

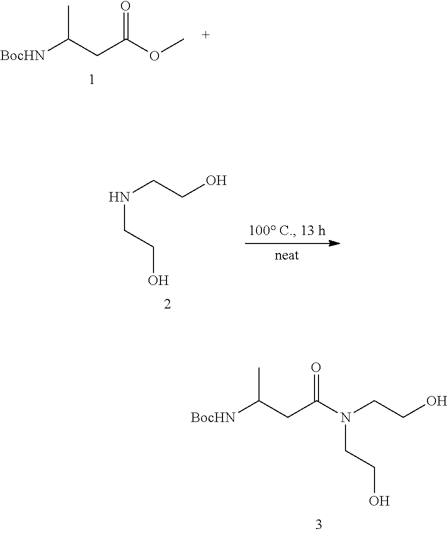 [SMILES: COC(=O)CC(C)NC(=O)OC(C)(C)C ; OCCNCCO ; CC(CC(=O)N(CCO)CCO)NC(=O)OC(C)(C)C]

A mixture of Compound 1 (2.06 g, 9.48 mmol, 1 eq.) and Compound 2 (996.86 mg, 9.48 mmol, 916.23 μL, 1 eq.) was heated to 100° C. for 13 hours. TLC (Dichloromethane:Methanol=10:1, R_{f}=0.3) indicated Compound 1 was consumed completely and one new spot formed. The mixture was purified by column chromatography (SiO₂, EtOAc to DCM:MeOH=5:1) to give Compound 3 (1.8 g, 6.20 mmol, 65.38% yield) as colorless oil. ¹H NMR (400 MHz, chloroform-d) δ=4.10 (m, 1H), 3.89 (m, 1H), 3.85-3.74 (m, 3H), 3.65-3.44 (m, 4H), 2.66-2.49 (m, 2H), 1.44 (s, 9H), 1.29-1.20 (m, 3H).

### Step 3: Lipid Coupling:

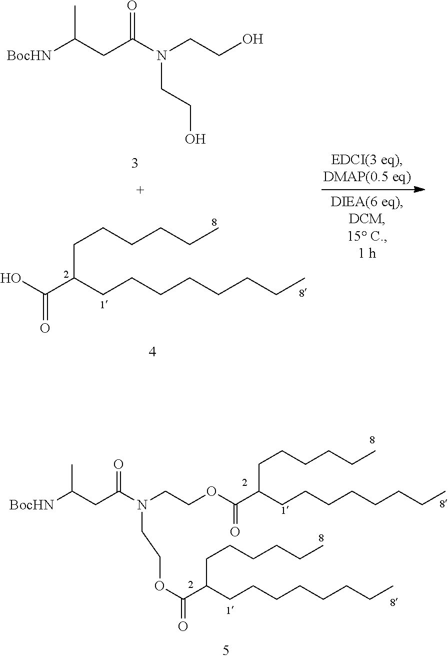 [SMILES: CC(CC(=O)N(CCO)CCO)NC(=O)OC(C)(C)C ; CCCCCCCCC(CCCCCC)C(=O)O ; CCCCCCCCC(CCCCCC)C(=O)OCCN(CCOC(=O)C(CCCCCC)CCCCCCCC)C(=O)CC(C)NC(=O)OC(C)(C)C]

To a solution of Compound 4 (2.21 g, 8.61 mmol, 2.5 eq.) in DCM (5 mL) was added DIEA (2.67 g, 20.66 mmol, 3.60 mL, 6 eq.), EDCI (1.98 g, 10.33 mmol, 3 eq.), DMAP (210.38 mg, 1.72 mmol, 0.5 eq.) and Compound 3 (1 g, 3.44 mmol, 1 eq.). The mixture was stirred at 15° C. for 1 h. TLC (Petroleum ether:Ethyl acetate=5:1, R_{f}=0.5) indicated Compound 3 was consumed completely and one new spot formed. The reaction mixture was concentrated under reduced pressure to give a residue. The residue was purified by column chromatography (SiO₂, PE:EtOAc=10:1) to give Compound 5 (2 g, 2.61 mmol, 75.70% yield) as a colorless oil.

Compound 5: LCMS [M−100+1]⁺=667.7. ¹H NMR (400 MHz, chloroform-d) δ=5.39 (br s, 1H), 4.26-4.11 (m, 4H), 4.09-3.97 (m, 1H), 3.72-3.51 (m, 4H), 2.72 (dd, J=4.4, 15.2 Hz, 1H), 2.49 (dd, J=6.0, 15.2 Hz, 1H), 2.32 (tt, J=5.6, 8.4 Hz, 2H), 1.61-1.52 (m, 4H), 1.44 (s, 12H), 1.35-1.14 (m, 41H), 0.88 (t, J=6.8 Hz, 12H).

### Step 4: Deprotection of Precursors/Synthesis of Cationic Species:

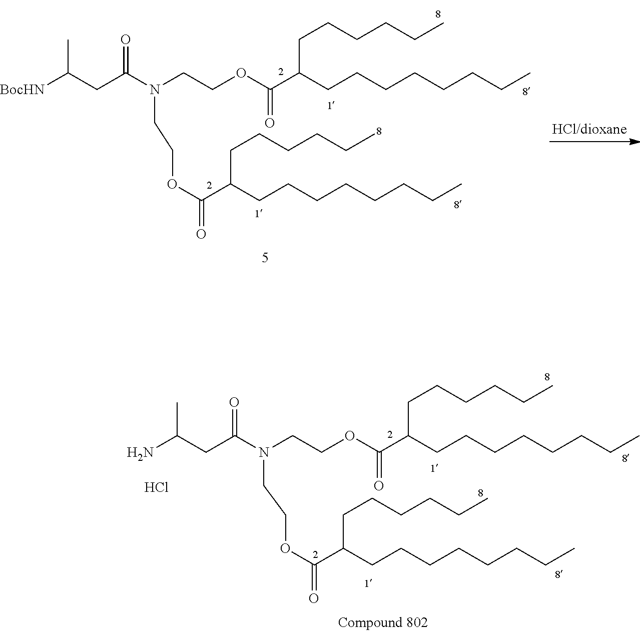 [SMILES: CCCCCCCCC(CCCCCC)C(=O)OCCN(CCOC(=O)C(CCCCCC)CCCCCCCC)C(=O)CC(C)NC(=O)OC(C)(C)C ; CCCCCCCCC(CCCCCC)C(=O)OCCN(CCOC(=O)C(CCCCCC)CCCCCCCC)C(=O)CC(C)N ; Cl]

A mixture of Compound 5 (700 mg, 912.44 μmol, 1 eq.) in HCl/dioxane (14 mL, 4 M) was degassed and purged with N₂ for 3 times, and then the mixture was stirred at 10° C. for 2 hours under N₂ atmosphere. LCMS showed Compound 5 was consumed completely and desired MS was detected. The reaction mixture was concentrated and lyophilized to give Compound 802 (500 mg, 749.56 μmol, 82.15% yield, HCl) as colorless oil.

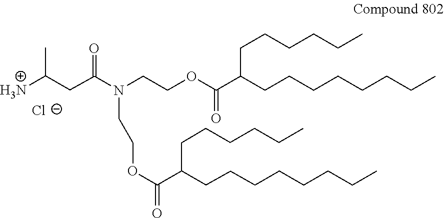 [SMILES: CCCCCCCCC(CCCCCC)C(=O)OCCN(CCOC(=O)C(CCCCCC)CCCCCCCC)C(=O)CC(C)[NH3+] ; [Cl-]]

LCMS [M+1]⁺=667.6. ¹H NMR (400 MHz, DMSO-d₆) δ=7.99 (br d, J=2.4 Hz, 3H), 4.18 (br d, J=2.4 Hz, 2H), 4.11 (t, J=6.0 Hz, 2H), 3.70-3.44 (m, 5H), 2.79-2.66 (m, 2H), 2.37-2.24 (m, 2H), 1.54-1.38 (m, 8H), 1.23 (br s, 43H), 0.86 (t, J=6.5 Hz, 12H).

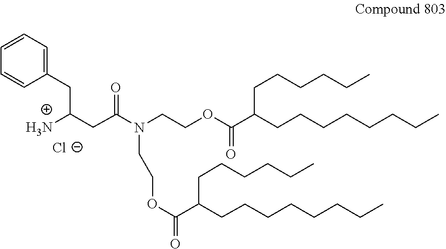 [SMILES: CCCCCCCCC(CCCCCC)C(=O)OCCN(CCOC(=O)C(CCCCCC)CCCCCCCC)C(=O)CC([NH3+])Cc1ccccc1 ; [Cl-]]

LCMS [M+1]⁺=743.6. ¹H NMR (400 MHz, DMSO-d6) δ=8.11 (br d, J=3.8 Hz, 3H), 7.36-7.29 (m, 2H), 7.29-7.23 (m, 3H), 4.21-3.96 (m, 4H), 3.71-3.50 (m, 4H), 3.46-3.42 (m, 1H), 3.08 (dd, J=5.5, 13.5 Hz, 1H), 2.83 (dd, J=8.8, 13.4 Hz, 1H), 2.79-2.69 (m, 1H), 2.68-2.58 (m, 1H), 2.35-2.23 (m, 1H), 2.05 (br d, J=5.0 Hz, 1H), 1.53-1.44 (m, 2H), 1.41-1.33 (m, 4H), 1.29-1.10 (m, 40H), 1.04 (br s, 2H), 0.89-0.80 (m, 12H).

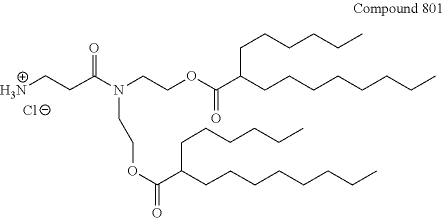 [SMILES: CCCCCCCCC(CCCCCC)C(=O)OCCN(CCOC(=O)C(CCCCCC)CCCCCCCC)C(=O)CC[NH3+] ; [Cl-]]

LCMS [M+1]⁺=653.6. ¹H NMR (400 MHz, DMSO-d₆) δ=7.95 (br s, 3H), 4.23-4.05 (m, 4H), 3.64-3.48 (m, 4H), 3.03-2.92 (m, 2H), 2.80-2.72 (m, 2H), 2.39-2.24 (m, 2H), 1.51-1.43 (m, 4H), 1.42-1.34 (m, 4H), 1.22 (br s, 40H), 0.85 (br t, J=6.5 Hz, 12H).

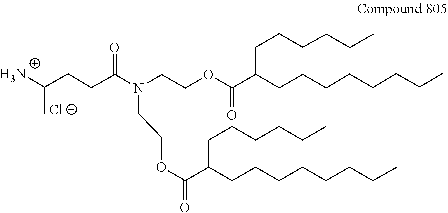 [SMILES: CCCCCCCCC(CCCCCC)C(=O)OCCN(CCOC(=O)C(CCCCCC)CCCCCCCC)C(=O)CCC(C)[NH3+] ; [Cl-]]

LCMS [M+1]⁺=681.6. ¹H NMR (400 MHz, DMSO-d₆) δ=8.19-7.82 (m, 3H), 4.23-4.01 (m, 4H), 3.81 (br s, 2H), 3.61 (br s, 2H), 3.51 (br t, J=5.2 Hz, 2H), 3.25-3.12 (m, 1H), 2.34-2.24 (m, 2H), 1.91-1.79 (m, 1H), 1.71-1.61 (m, 1H), 1.47 (m, 4H), 1.42-1.34 (m, 4H), 1.30-1.12 (m, 43H), 0.85 (br t, J=6.4 Hz, 12H).

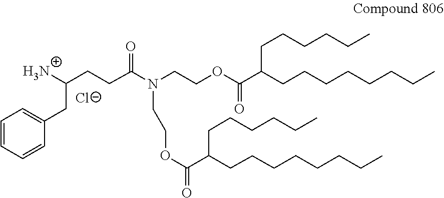 [SMILES: CCCCCCCCC(CCCCCC)C(=O)OCCN(CCOC(=O)C(CCCCCC)CCCCCCCC)C(=O)CCC([NH3+])Cc1ccccc1 ; [Cl-]]

LCMS [M+1]⁺=757.6. ¹H NMR (400 MHz, DMSO-d6) δ=8.12 (m, 3H), 7.37-7.30 (m, 2H), 7.29-7.21 (m, 3H), 4.18-4.09 (m, 2H), 4.05 (br t, J=5.6 Hz, 2H), 3.61-3.51 (m, 2H), 3.47 (m, 2H), 3.39 (br s, 1H), 3.05-2.97 (m, 1H), 2.84-2.75 (m, 1H), 2.60-2.52 (m, 1H), 2.48-2.40 (m, 1H), 2.32-2.20 (m, 2H), 1.78-1.67 (m, 2H), 1.52-1.42 (m, 4H), 1.41-1.33 (m, 4H), 1.21 (br s, 40H), 0.92-0.78 (m, 12H).

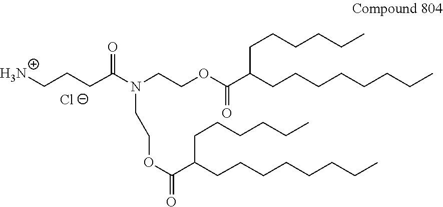 [SMILES: CCCCCCCCC(CCCCCC)C(=O)OCCN(CCOC(=O)C(CCCCCC)CCCCCCCC)C(=O)CCC[NH3+] ; [Cl-]]

LCMS [M+1]⁺=667.8. ¹H NMR (400 MHz, DMSO-d₆) δ=7.93 (br s, 3H), 4.17 (s, 2H), 4.11-4.03 (m, 2H), 3.59 (br t, J=5.6 Hz, 2H), 3.51 (s, 2H), 2.79 (br d, J=5.8 Hz, 2H), 2.49-2.43 (m, 2H), 2.35-2.23 (m, 2H), 1.85-1.72 (m, 2H), 1.52-1.33 (m, 8H), 1.22 (br s, 40H), 0.85 (br t, J=6.4 Hz, 12H).

### Dimethyl Lipid Series:

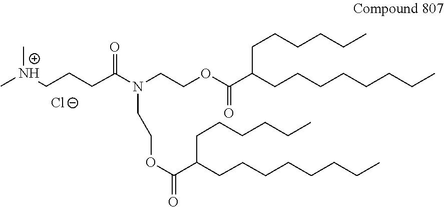 [SMILES: CCCCCCCCC(CCCCCC)C(=O)OCCN(CCOC(=O)C(CCCCCC)CCCCCCCC)C(=O)CCC[NH+](C)C ; [Cl-]]

LCMS [M+1]⁺=695.6. ¹H NMR (400 MHz, DMSO-d₆) δ=9.91 (br s, 1H), 4.17 (br t, J=5.6 Hz, 2H), 4.09 (t, J=6.0 Hz, 2H), 3.59 (br t, J=5.6 Hz, 2H), 3.51 (br t, J=6.0 Hz, 2H), 3.06-2.98 (m, 2H), 2.74 (s, 6H), 2.47-2.44 (m, 1H), 2.35-2.24 (m, 2H), 1.90-1.79 (m, 2H), 1.51-1.34 (m, 8H), 1.22 (br s, 41H), 0.85 (t, J=6.4 Hz, 12H).

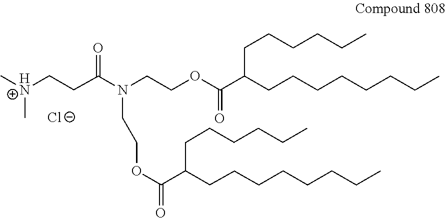

LCMS [M+1]⁺=681.6. ¹H NMR (400 MHz, DMSO-d₆) δ=9.72-9.32 (m, 1H), 4.24-4.05 (m, 4H), 3.67-3.47 (m, 4H), 3.26 (br t, J=6.8 Hz, 2H), 2.87 (br t, J=7.2 Hz, 2H), 2.77 (s, 6H), 2.35-2.26 (m, 2H), 1.53-1.37 (m, 8H), 1.22 (br s, 40H), 0.85 (t, J=6.4 Hz, 12H).

### Example 6. Synthesis and Characterization of Series 900 Lipidoids

### General Procedure for Preparation of Boc-Protected Diethanolamine Lipids

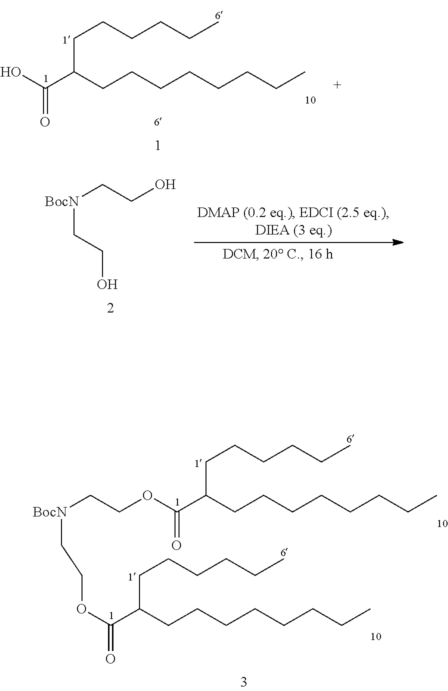 [SMILES: CCCCCCCCC(CCCCCC)C(=O)O ; CC(C)(C)OC(=O)N(CCO)CCO ; CCCCCCCCC(CCCCCC)C(=O)OCCN(CCOC(=O)C(CCCCCC)CCCCCCCC)C(=O)OC(C)(C)C]

To a solution of Compound 1 (13.74 g, 53.59 mmol, 2.2 eq.) in DCM (50 mL) was added DMAP (595.21 mg, 4.87 mmol, 0.2 eq.), EDCI (11.67 g, 60.90 mmol, 2.5 eq.), DIEA (9.45 g, 73.08 mmol, 12.73 mL, 3 eq.) and Compound 2 (5 g, 24.36 mmol, 4.61 mL, 1 eq.). The mixture was stirred at 20° C. for 16 hours. TLC (Petroleum ether:Ethyl acetate=3:1, P1:R_{f}=0.64) indicated Compound 1 and Compound 2 was consumed completely and new spots formed. The reaction mixture was quenched by addition sat. NaHCO₃ (10 mL) at 20° C., and then diluted with H₂O (40 mL) and extracted with DCM (80 mL). The combined organic layers were washed with brine (30 mL), dried over Na₂SO₄, filtered and concentrated under reduced pressure to give a residue. The residue was purified by column chromatography (SiO₂, Petroleum ether:Ethyl acetate=200:1 to 70:1 to 30:1) to give Compound 3 (11.48 g, 16.81 mmol, 52.13% yield, 99.9% purity) as colorless oil.

Compound 3: LCMS [M−100+1]⁺=582.5. ¹H NMR (400 MHz, chloroform-d) δ=4.33-4.07 (m, 4H), 3.66-3.31 (m, 4H), 2.54-2.18 (m, 2H), 1.64-1.55 (m, 4H), 1.50-1.38 (m, 13H), 1.34-1.19 (m, 40H), 0.88 (t, J=6.8 Hz, 12H).

### General Procedure for Preparation of Deprotected Diethanolamine Lipids

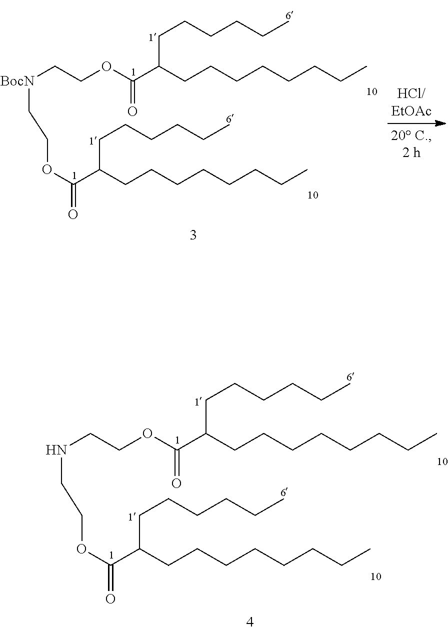 [SMILES: CCCCCCCCC(CCCCCC)C(=O)OCCN(CCOC(=O)C(CCCCCC)CCCCCCCC)C(=O)OC(C)(C)C ; CCCCCCCCC(CCCCCC)C(=O)OCCNCCOC(=O)C(CCCCCC)CCCCCCCC]

A solution of Compound 3 (9.48 g, 13.90 mmol, 1 eq.) in HCl/EtOAc (4 M, 47.40 mL, 13.64 eq.) was stirred at 20° C. for 2 hours. LC-MS showed Compound 3 was consumed and desired MS was detected. The reaction mixture was concentrated under reduced pressure to give Compound 4 (8.6 g, crude, HCl) as colorless oil.

Compound 4: LCMS [M+1]⁺=582.5. ¹H NMR (400 MHz, chloroform-d) δ=9.87 (br s, 2H), 4.51 (br s, 4H), 3.36 (br s, 4H), 2.52-2.31 (m, 2H), 1.71-1.56 (m, 4H), 1.53-1.41 (m, 4H), 1.38-1.18 (m, 40H), 0.88 (t, J=6.8 Hz, 12H).

### General Procedure for Preparation of Acrylate Precursor

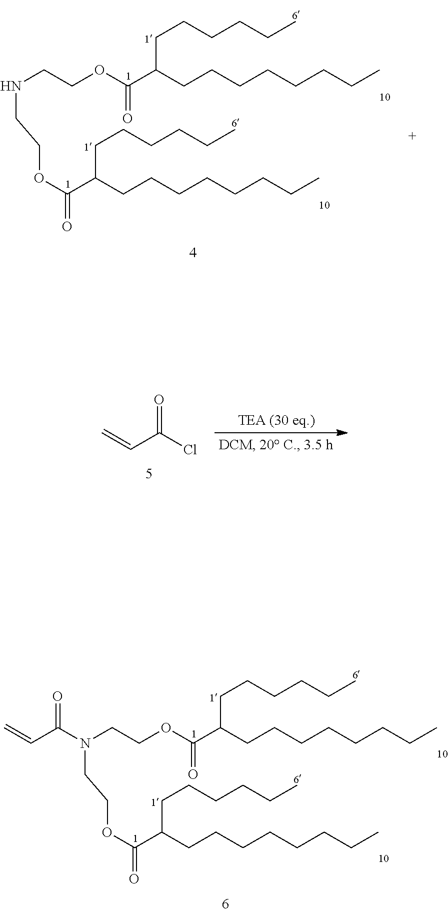

To a solution of Compound 4 (8.6 g, 13.91 mmol, 1 eq., HCl) in DCM (90 mL) was added TEA (42.22 g, 417.20 mmol, 58.07 mL, 30 eq.) and Compound 5 (1.76 g, 19.47 mmol, 1.58 mL, 1.4 eq.) at 0° C. The mixture was stirred at 25° C. for 16 hours. TLC (Petroleum ether:Ethyl acetate=5:1, P1:R_{f}=0.50) indicated Compound 4 was consumed completely and new spots formed. The reaction mixture was quenched by addition MeOH (15 mL) at 20° C., and then washed with NH₄Cl (50 mL). The organic layer was washed with brine (30 mL), dried over Na₂SO₄, filtered and concentrated under reduced pressure to give a residue. The residue was purified by column chromatography (SiO₂, Petroleum ether:Ethyl acetate=50:1 to 26:1) to give Compound 6 (3 g, 4.69 mmol, 33.75% yield, 99.5% purity) as colorless oil.

Compound 6: LCMS [M+23]⁺=658.5. ¹H NMR (400 MHz, chloroform-d) δ=6.65 (dd, J=10.4, 16.8 Hz, 1H), 6.40 (dd, J=2.0, 16.8 Hz, 1H), 5.74 (dd, J=2.0, 10.4 Hz, 1H), 4.34-4.13 (m, 4H), 3.69 (t, J=5.6 Hz, 4H), 2.43-2.24 (m, 2H), 1.62-1.51 (m, 4H), 1.49-1.38 (m, 4H), 1.25 (br s, 40H), 0.88 (t, J=6.8 Hz, 12H)

### General Procedure for Preparation of Series 900-1

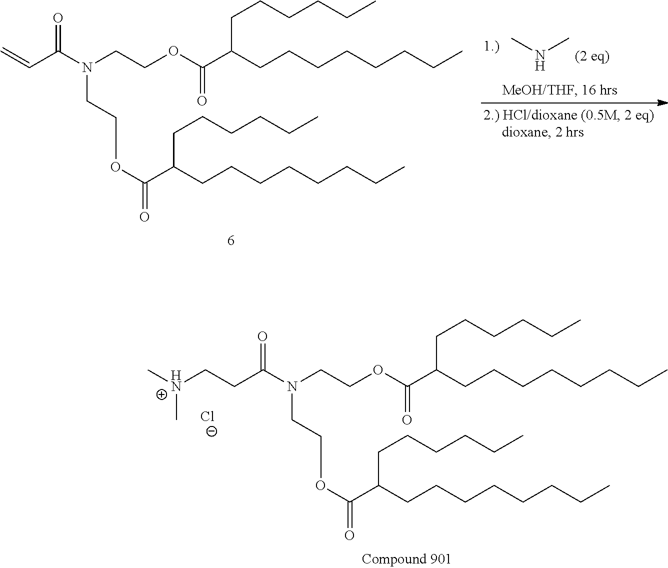

To a solution of Compound 6 (50 mg, 78.62 μmol, 1 eq.) in MeOH (1.5 mL) and THF (1.5 mL) was added N-methylmethanamine (2 M, 78.62 μL, 2 eq.). The mixture was stirred at 20° C. for 16 hours. LC-MS showed Compound 6 was consumed completely and one main peak with desired mass was detected. The reaction mixture was concentrated under reduced pressure to give a residue. The residue was purified by column chromatography (SiO₂, DCM:MeOH=1:0 to 50:1) to give Compound 901 (40 mg, 58.73 μmol, 74.70% yield, 100% purity) as colorless oil.

Compound 901 LCMS [M+1]+=681.6. ¹H NMR (400 MHz, chloroform-d) δ=4.21 (q, J=5.6 Hz, 4H), 3.75-3.53 (m, 4H), 3.03 (br d, J=2.8 Hz, 2H), 2.85 (br s, 2H), 2.54 (br s, 6H), 2.33 (br dd, J=5.6, 8.0 Hz, 2H), 1.65-1.52 (m, 4H), 1.49-1.40 (m, 4H), 1.26 (br s, 40H), 0.88 (br t, J=6.8 Hz, 12H)

### General Procedure for Preparation of HCl Salt

To a solution of Compound 901 (40 mg, 58.73 μmol, 1 eq.) in dioxane (1 mL) was added HCl/dioxane (0.5 M, 234.92 μL, 2 eq.). The mixture was stirred at 20° C. for 2 hours. The mixture was concentrated and lyophilized to give Compound 901 (27.8 mg, 38.74 μmol, 65.97% yield, HCl) as colorless oil.

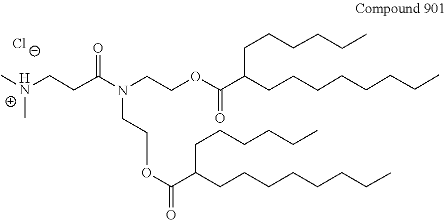

LCMS [M+1]⁺=681.6. ¹H NMR (400 MHz, chloroform-d) δ=12.32 (br s, 1H), 4.31-4.13 (m, 4H), 3.75-3.56 (m, 4H), 3.43 (br s, 2H), 3.14 (br s, 2H), 2.84 (br s, 6H), 2.46-2.22 (m, 2H), 1.63-1.52 (m, 4H), 1.45 (m, 4H), 1.26 (br s, 40H), 0.88 (t, J=6.8 Hz, 12H)

Compound 902 and 903 have varied lipid tails than the other structures. They were prepared with the same method as Compound 901, but using different lipid starting materials.

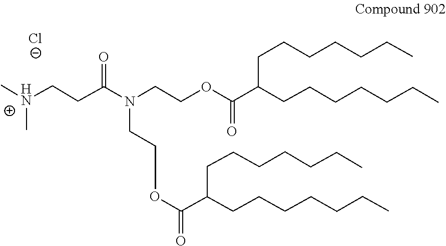

LCMS [M+1]⁺=681.6. ¹H NMR (400 MHz, chloroform-d) δ=12.67-12.40 (m, 1H), 4.34-4.08 (m, 4H), 3.75-3.55 (m, 4H), 3.47-3.35 (m, 2H), 3.27-3.06 (m, 2H), 2.94-2.73 (m, 6H), 2.45-2.26 (m, 2H), 1.62-1.53 (m, 4H), 1.49-1.40 (m, 4H), 1.34-1.20 (m, 40H), 0.95-0.78 (m, 12H)

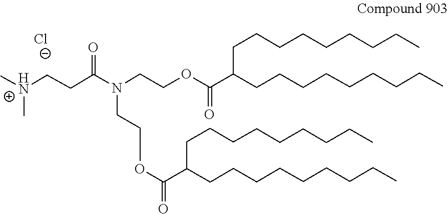

LCMS [M+1]⁺=793.7. ¹H NMR (400 MHz, chloroform-d) δ=12.75-12.46 (m, 1H), 4.22 (td, J=5.6, 15.0 Hz, 4H), 3.72-3.56 (m, 4H), 3.42 (br d, J=5.4 Hz, 2H), 3.15 (br t, J=6.4 Hz, 2H), 2.82 (d, J=4.8 Hz, 6H), 2.40-2.26 (m, 2H), 1.61-1.52 (m, 4H), 1.45 (br dd, J=5.2, 8.4 Hz, 4H), 1.26 (s, 56H), 0.88 (t, J=6.8 Hz, 12H).

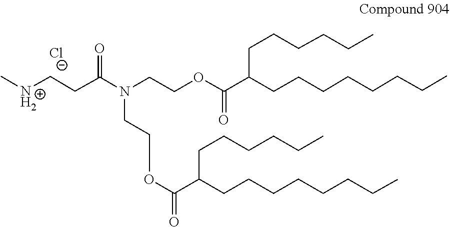 [SMILES: CCCCCCCCC(CCCCCC)C(=O)OCCN(CCOC(=O)C(CCCCCC)CCCCCCCC)C(=O)CC[NH2+]C ; [Cl-]]

LCMS [M+1]⁺=667.6. ¹H NMR (400 MHz, chloroform-d) δ=9.63-9.38 (m, 2H), 4.22 (br s, 4H), 3.61 (br s, 4H), 3.27 (br s, 2H), 3.12 (br s, 2H), 2.79 (br s, 3H), 2.40-2.28 (m, 2H), 1.65-1.52 (m, 4H), 1.49-1.40 (m, 4H), 1.35-1.19 (m, 40H), 0.89 (t, J=6.8 Hz, 12H).

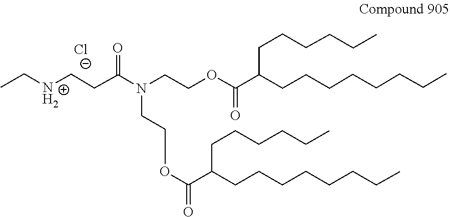 [SMILES: CCCCCCCCC(CCCCCC)C(=O)OCCN(CCOC(=O)C(CCCCCC)CCCCCCCC)C(=O)CC[NH2+]CC ; [Cl-]]

LCMS [M+1]⁺=681.6. ¹H NMR (400 MHz, chloroform-d) δ=9.38 (br s, 2H), 4.22 (br t, J=5.6 Hz, 4H), 3.68-3.54 (m, 4H), 3.25 (br s, 2H), 3.19-3.04 (m, 4H), 2.41-2.29 (m, 2H), 1.62-1.52 (m, 4H), 1.51-1.41 (m, 7H), 1.36-1.16 (m, 40H), 0.88 (t, J=6.8 Hz, 12H).

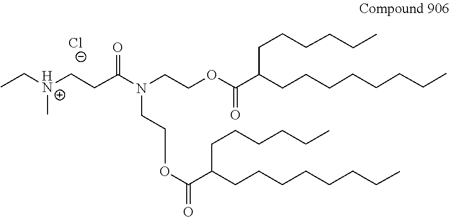

LCMS [M+1]⁺=695.6. ¹H NMR (400 MHz, chloroform-d) δ=11.98 (br s, 1H), 4.22 (td, J=5.6, 19.6 Hz, 4H), 3.74-3.56 (m, 4H), 3.48 (m, 1H), 3.38-3.02 (m, 5H), 2.77 (d, J=4.6 Hz, 3H), 2.38-2.27 (m, 2H), 1.65-1.52 (m, 4H), 1.51-1.41 (m, 7H), 1.35-1.17 (m, 40H), 0.88 (t, J=6.8 Hz, 12H).

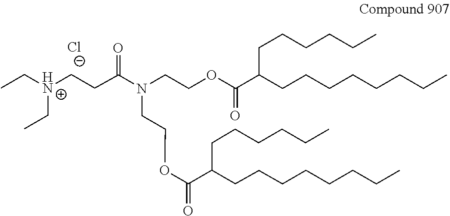

LCMS [M+1]⁺=709.6. ¹H NMR (400 MHz, chloroform-d) δ=12.15 (br s, 1H), 4.22 (td, J=5.6, 18.8 Hz, 4H), 3.70 (br t, J=5.6 Hz, 2H), 3.61 (t, J=5.6 Hz, 2H), 3.37 (br d, J=5.2 Hz, 2H), 3.27-3.14 (m, 4H), 3.14-3.02 (m, 2H), 2.33 (ddd, J=2.9, 5.6, 8.4 Hz, 2H), 1.63-1.51 (m, 4H), 1.50-1.38 (m, 10H), 1.35-1.15 (m, 40H), 0.98-0.81 (m, 12H).

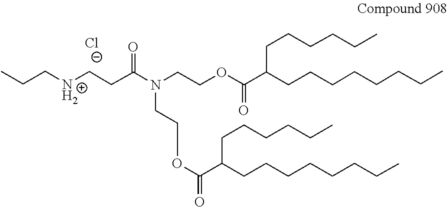 [SMILES: CCCCCCCCC(CCCCCC)C(=O)OCCN(CCOC(=O)C(CCCCCC)CCCCCCCC)C(=O)CC[NH2+]CCC ; [Cl-]]

LCMS [M+1]⁺=695.6. ¹H NMR (400 MHz, chloroform-d) δ=9.38-9.18 (m, 2H), 4.28-4.16 (m, 4H), 3.67-3.56 (m, 4H), 3.26 (br s, 2H), 3.19-3.09 (m, 2H), 3.06-2.95 (m, 2H), 2.38-2.29 (m, 2H), 1.94-1.84 (m, 2H), 1.64-1.52 (m, 4H), 1.46 (br d, J=4.8 Hz, 4H), 1.34-1.18 (m, 40H), 1.06 (t, J=7.2 Hz, 3H), 0.97-0.77 (m, 12H).

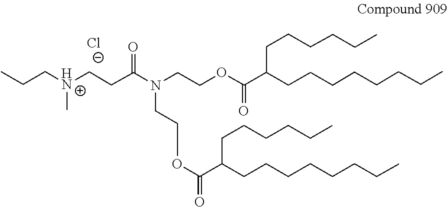

LCMS [M+1]⁺=709.6. ¹H NMR (400 MHz, chloroform-d) δ=11.97 (br s, 1H), 4.32-4.14 (m, 4H), 3.74-3.56 (m, 4H), 3.49 (m, 1H), 3.34 (m, 1H), 3.18 (m, 2H), 3.14-3.01 (m, 1H), 2.93 (br dd, J=5.6, 12.0 Hz, 1H), 2.79 (d, J=4.8 Hz, 3H), 2.40-2.28 (m, 2H), 2.03-1.82 (m, 2H), 1.57 (br dd, J=7.6, 13.6 Hz, 4H), 1.46 (br dd, J=6.0, 8.0 Hz, 4H), 1.36-1.16 (m, 40H), 1.04 (t, J=7.2 Hz, 3H), 0.88 (t, J=6.8 Hz, 12H).

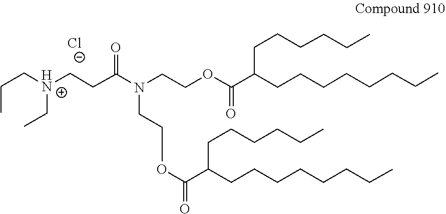

LCMS [M+1]⁺=723.6. ¹H NMR (400 MHz, chloroform-d) δ=12.31-12.17 (m, 1H), 4.28-4.16 (m, 4H), 3.70 (br t, J=6.0 Hz, 2H), 3.64-3.57 (m, 2H), 3.43-3.35 (m, 2H), 3.25-3.16 (m, 3H), 3.14-2.89 (m, 3H), 2.39-2.29 (m, 2H), 1.94-1.81 (m, 2H), 1.56-1.51 (m, 3H), 1.50-1.38 (m, 8H), 1.33-1.16 (m, 40H), 1.03 (t, J=7.6 Hz, 3H), 0.89 (t, J=6.8 Hz, 12H).

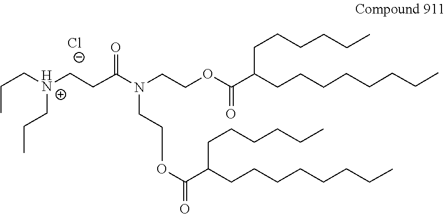

LCMS [M+1]⁺=737.7. 1H NMR (400 MHz, chloroform-d) δ=12.31-12.11 (m, 1H), 4.31-4.11 (m, 4H), 3.69 (br t, J=5.6 Hz, 2H), 3.63-3.55 (m, 2H), 3.43-3.33 (m, 2H), 3.27-3.16 (m, 2H), 3.06-2.88 (m, 4H), 2.40-2.27 (m, 2H), 1.95-1.80 (m, 4H), 1.58-1.49 (m, 4H), 1.49-1.37 (m, 4H), 1.33-1.18 (m, 40H), 1.07-0.97 (m, 6H), 0.89 (t, J=6.8 Hz, 12H)

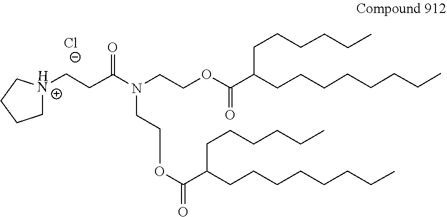

LCMS [M+1]⁺=707.6. 1H NMR (400 MHz, chloroform-d) δ=12.58 (br s, 1H), 4.21 (td, J=5.6, 15.1 Hz, 4H), 3.80-3.65 (m, 4H), 3.61 (t, J=5.6 Hz, 2H), 3.45 (br d, J=5.6 Hz, 2H), 3.16 (br t, J=6.4 Hz, 2H), 2.87 (br d, J=6.4 Hz, 2H), 2.33 (dt, J=2.8, 5.6 Hz, 2H), 2.28-2.16 (m, 2H), 2.09 (br s, 2H), 1.60-1.52 (m, 4H), 1.46 (br dd, J=5.6, 8.4 Hz, 4H), 1.36-1.20 (m, 40H), 0.88 (t, J=6.8 Hz, 12H).

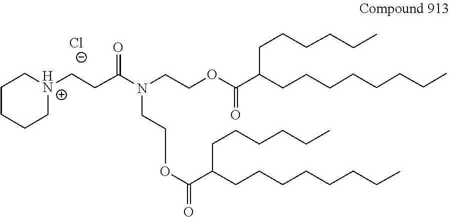

LCMS [M+1]⁺=721.6. 1H NMR (400 MHz, chloroform-d) δ=12.24-12.00 (m, 1H), 4.22 (td, J=5.6, 17.6 Hz, 4H), 3.69 (br t, J=5.6 Hz, 2H), 3.61 (t, J=5.6 Hz, 2H), 3.50 (br d, J=11.6 Hz, 2H), 3.34 (q, J=5.6 Hz, 2H), 3.27-3.16 (m, 2H), 2.75-2.60 (m, 2H), 2.41-2.18 (m, 4H), 1.99-1.81 (m, 3H), 1.57 (br dd, J=7.2, 14.4 Hz, 4H), 1.50-1.38 (m, 5H), 1.35-1.16 (m, 40H), 0.88 (t, J=6.8 Hz, 12H).

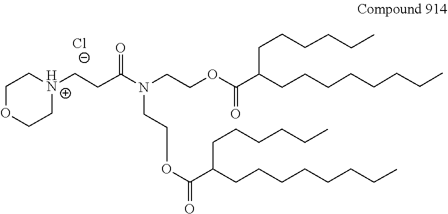

LCMS [M+1]⁺=723.6. 1H NMR (400 MHz, chloroform-d) δ=13.10 (br s, 1H), 4.31-4.16 (m, 6H), 3.98 (dd, J=2.8, 12.8 Hz, 2H), 3.67 (br t, J=5.6 Hz, 2H), 3.60 (t, J=5.6 Hz, 2H), 3.45-3.36 (m, 4H), 3.26-3.19 (m, 2H), 3.03-2.86 (m, 2H), 2.38-2.27 (m, 2H), 1.62-1.52 (m, 4H), 1.50-1.39 (m, 4H), 1.33-1.18 (m, 40H), 0.88 (t, J=6.8 Hz, 12H).

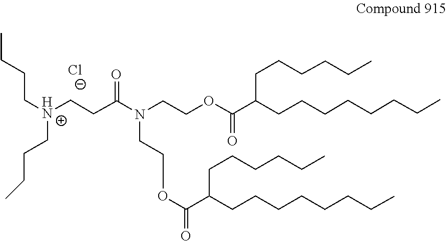

LCMS [M+1]⁺=765.7. 1H NMR (400 MHz, chloroform-d) δ=12.12 (br s, 1H), 4.28-4.15 (m, 4H), 3.73-3.66 (m, 2H), 3.60 (br t, J=5.6 Hz, 2H), 3.37 (br d, J=3.6 Hz, 2H), 3.21 (br s, 2H), 3.01 (dt, J=5.6, 12.0 Hz, 4H), 2.38-2.28 (m, 2H), 1.79 (br dd, J=5.6, 11.2 Hz, 4H), 1.57 (td, J=7.2, 14.0 Hz, 4H), 1.49-1.36 (m, 8H), 1.35-1.17 (m, 40H), 0.99 (t, J=7.2 Hz, 6H), 0.88 (t, J=6.8 Hz, 12H).

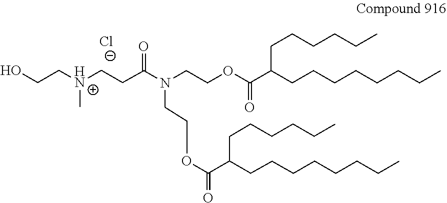

LCMS [M+1]⁺=711.6. ¹H NMR (400 MHz, chloroform-d) δ=11.64 (br s, 1H), 4.38-4.13 (m, 4H), 4.02 (br s, 2H), 3.76-3.56 (m, 5H), 3.41 (br s, 2H), 3.18 (br s, 3H), 2.90 (br d, J=2.8 Hz, 3H), 2.42-2.28 (m, 3H), 1.64-1.51 (m, 4H), 1.49-1.40 (m, 4H), 1.35-1.18 (m, 40H), 0.88 (t, J=6.8 Hz, 12H)

### Series 900-2

### Synthesis and Characterization Data for Compounds 921-929, 970-976

### Route:

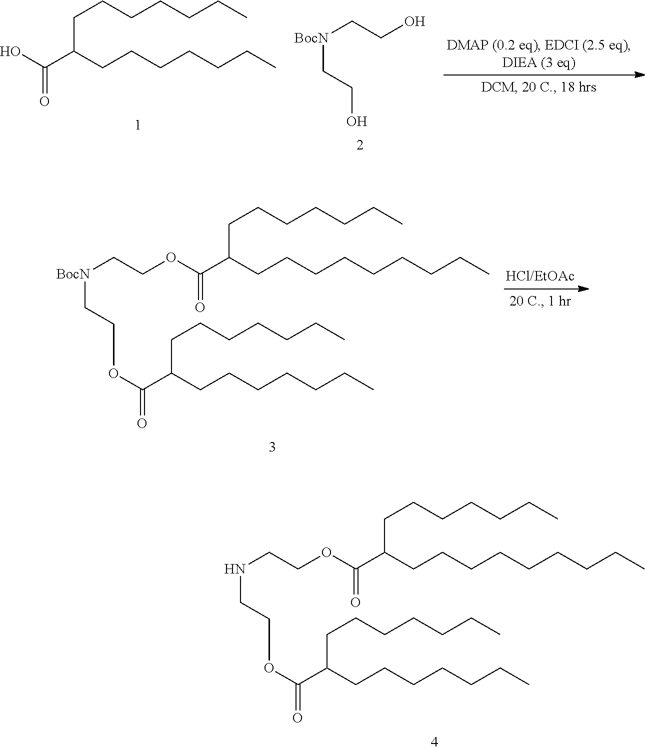 [SMILES: CCCCCCCC(CCCCCCC)C(=O)O ; CC(C)(C)OC(=O)N(CCO)CCO ; CCCCCCCCCC(CCCCCCC)C(=O)OCCN(CCOC(=O)C(CCCCCCC)CCCCCCC)C(=O)OC(C)(C)C ; CCCCCCCCCC(CCCCCCC)C(=O)OCCNCCOC(=O)C(CCCCCCC)CCCCCCC]

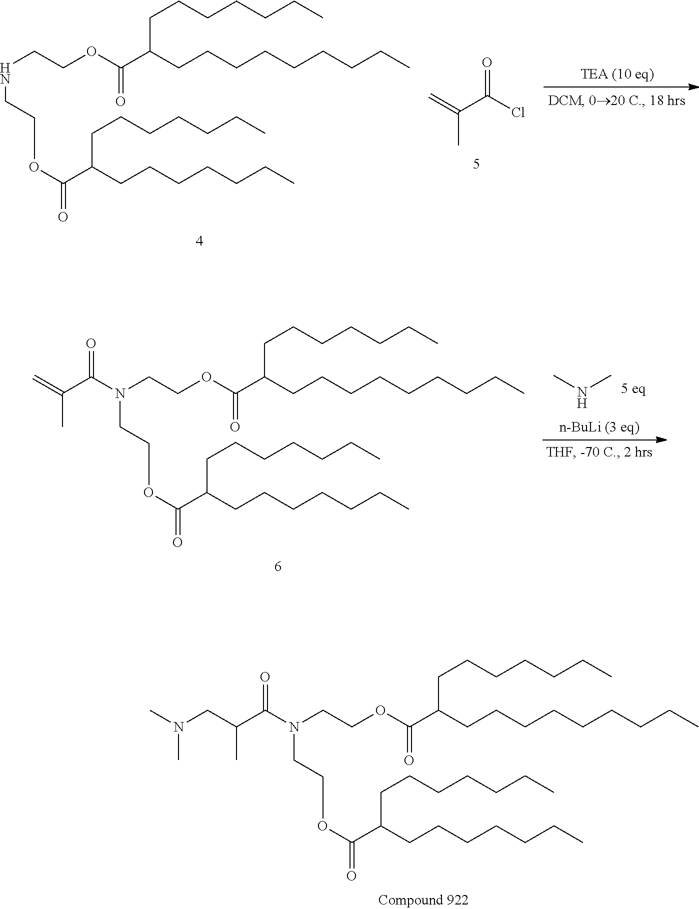

### General Synthetic Procedure for Preparation of Protected Lipidated Diethanolamine:

### Procedure for Preparation of Compound 3:

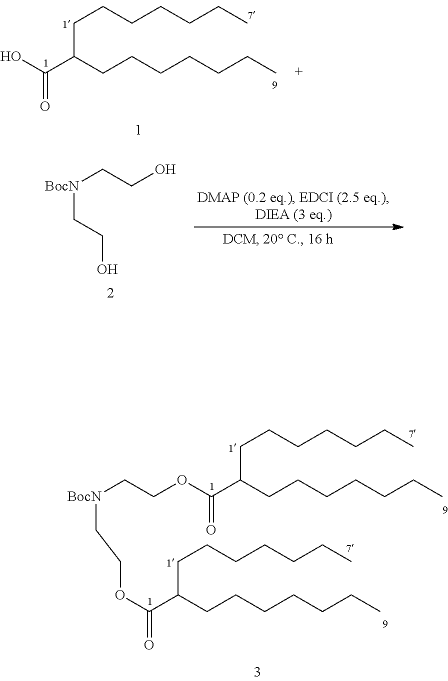 [SMILES: CCCCCCCC(CCCCCCC)C(=O)O ; CC(C)(C)OC(=O)N(CCO)CCO ; CCCCCCCC(CCCCCCC)C(=O)OCCN(CCOC(=O)C(CCCCCCC)CCCCCCC)C(=O)OC(C)(C)C]

To a solution of Compound 1 (1.00 g, 4.87 mmol, 922.16 μL, 1 eq.) and Compound 2 (2.75 g, 10.72 mmol, 2.2 eq.) in DCM (10 mL) was added EDCI (2.34 g, 12.19 mmol, 2.5 eq.), DMAP (119.11 mg, 974.95 mol, 0.2 eq.) and DIEA (1.89 g, 14.62 mmol, 2.55 mL, 3 eq.). The mixture was stirred at 20° C. for 16 hours. LCMS showed Compound 1 was consumed completely and one main peak with desired mass was detected. The reaction mixture was diluted with sat.NH₄Cl (10 mL) and extracted with DCM (30 mL). The combined organic layers were dried over Na₂SO₄, filtered and concentrated under reduced pressure to give a residue. The residue was purified by column chromatography (SiO₂, Petroleum ether:Ethyl acetate:Dichloromethane=400:1:1 to 60:1:1) to give Compound 3 (2.1 g, 3.08 mmol, 63.16% yield, 100% purity) as colorless oil.

### LCMS [M−100+1]⁺=582.5

¹H NMR (400 MHz, chloroform-d) δ=4.23-4.10 (m, 4H), 3.60-3.41 (m, 4H), 2.38-2.27 (m, 2H), 1.60-1.56 (m, 4H), 1.51-1.39 (m, 13H), 1.32-1.21 (m, 40H), 0.96-0.79 (m, 12H)

### General Deprotection Procedure for Lipidated Diethanolamines: Procedure for Preparation of Compound 4

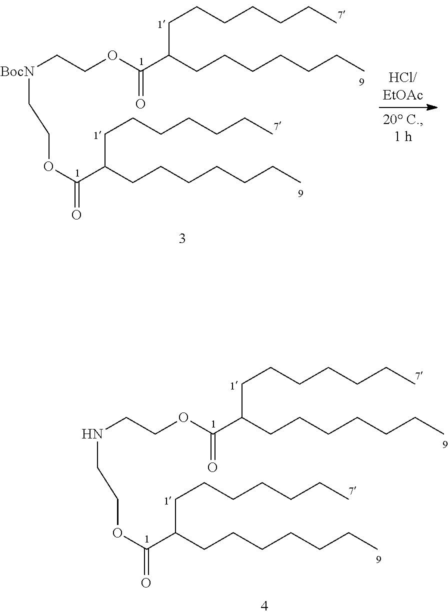 [SMILES: CCCCCCCC(CCCCCCC)C(=O)OCCN(CCOC(=O)C(CCCCCCC)CCCCCCC)C(=O)OC(C)(C)C ; CCCCCCCC(CCCCCCC)C(=O)OCCNCCOC(=O)C(CCCCCCC)CCCCCCC]

A solution of Compound 3 (2 g, 2.93 mmol, 1 eq.) in HCl/EtOAc (4 M, 10 mL, 13.64 eq.) was stirred at 20° C. for 1 hour. LCMS showed Compound 3 was consumed completely and one main peak with desired mass was detected. The mixture was concentrated to give Compound 4 (2 g, crude) as colorless oil.

### LCMS [M+1]⁺=582.5

¹H NMR (400 MHz, chloroform-d) δ=10.15-10.02 (m, 2H), 4.52 (br s, 4H), 3.35 (br s, 4H), 2.51-2.32 (m, 2H), 1.67-1.56 (m, 4H), 1.54-1.43 (m, 4H), 1.33-1.23 (m, 40H), 0.94-0.83 (m, 12H)

### General Procedure for Preparation of Methacrylate Precursor: Procedure for Preparation of Compound 6

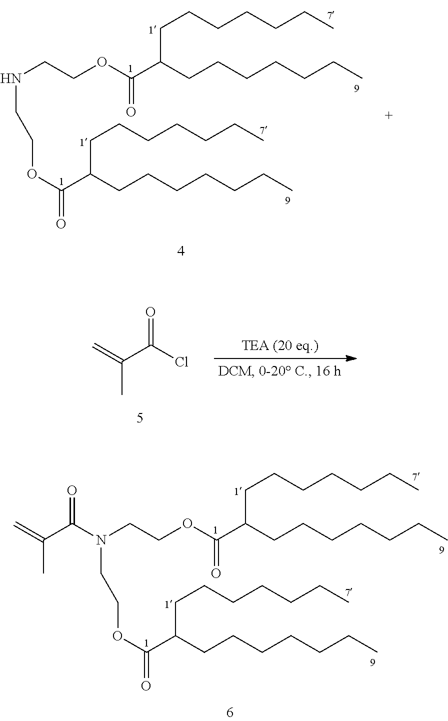

To a mixture of Compound 4 (1 g, 1.72 mmol, 1 eq.) and TEA (3.48 g, 34.37 mmol, 4.78 mL, 20 eq.) in DCM (10 mL) was added Compound 5 (269.44 mg, 2.58 mmol, 251.82 μL, 1.5 eq.) at 0° C. The mixture was stirred at 20° C. for 16 hours. LCMS showed Compound 4 was consumed completely and one main peak with desired mass was detected. The reaction mixture was diluted with sat.NH₄Cl (20 mL) and extracted with DCM (60 mL). The combined organic layers were dried over Na₂SO₄, filtered and concentrated under reduced pressure to give a residue. The residue was purified by column chromatography (SiO₂, Petroleum ether:Ethyl acetate=100:1 to 10:1) to give Compound 6 (1 g, 1.54 mmol, 89.53% yield, 100% purity) as colorless oil.

¹H NMR (400 MHz, chloroform-d) δ=5.30-5.17 (m, 1H), 5.06 (s, 1H), 4.22 (s, 4H), 3.70 (t, J=6.0 Hz, 4H), 2.40-2.27 (m, 2H), 1.98 (s, 3H), 1.60-1.52 (m, 4H), 1.49-1.40 (m, 4H), 1.35-1.17 (m, 40H), 0.88 (t, J=6.8 Hz, 12H)

### Representative Preparation (Compounds 921-929, 970-976): Procedure for Preparation of Compound 922

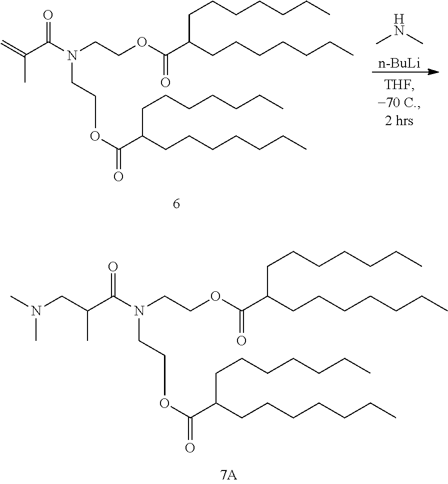

To a solution of N-methylmethanamine (2 M, 1.15 mL, 5 eq.) in THF (3 mL) was added n-BuLi (2.5 M, 553.83 μL, 3 eq.) at −70° C. for 30 min. Then Compound 6 (300 mg, 461.52 μmol, 1 eq.) in THF (0.5 mL) was added dropwise at −70° C. The mixture was stirred at −70° C. for 2 hours. LCMS showed Compound 6 was consumed completely and one main peak with desired mass was detected. The reaction mixture was quenched by addition NH₄Cl (5 mL) at −70° C., and then extracted with DCM (24 mL). The combined organic layers were washed with brine (5 mL), dried over Na₂SO₄, filtered and concentrated under reduced pressure to give a residue. The residue was purified by column chromatography (SiO₂, Dichloromethane:Methanol=30:1 to 25:1) to give the intermediate of target Compound 7A (34 mg, 46.96 μmol, 10.17% yield, 96% purity) as yellow oil. ¹H NMR (400 MHz, chloroform-d) δ=4.35-4.14 (m, 4H), 4.04-3.37 (m, 6H), 3.23-2.91 (m, 1H), 2.21 (d, J=18.0 Hz, 8H), 1.59-1.51 (m, 4H), 1.50-1.41 (m, 4H), 1.32-1.22 (m, 40H), 1.18-1.10 (m, 3H), 0.88 (t, J=6.8 Hz, 12H)

### Representative Procedure for Synthesizing the HCl Salts of Compounds 921-929, 970-976

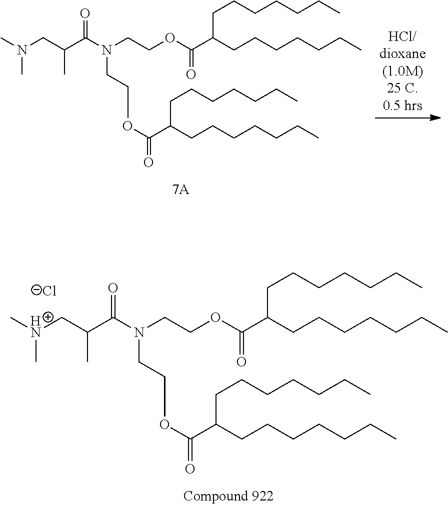

A solution of compound 7A (0.034 g, 48.91 μmol, 1 eq.) in HCl (11.22 g, 123.09 μmol, 11 mL, 0.04 wt %, 2.52 eq.) was stirred at 20° C. for 30 mins. The mixture was lyophilized to give Compound 922 (34 mg, 46.48 μmol, 95.02% yield, HCl) as colorless oil.

### LCMS [M+1]⁺=695.7

¹H NMR (400 MHz, chloroform-d) δ=12.64-12.43 (m, 1H), 4.38 (t, J=6.0 Hz, 2H), 4.28-4.16 (m, 2H), 4.00-3.90 (m, 1H), 3.82-3.62 (m, 4H), 3.60-3.48 (m, 1H), 2.96 (d, J=12.0 Hz, 1H), 2.83 (d, J=4.4 Hz, 3H), 2.69 (d, J=4.4 Hz, 3H), 2.39-2.27 (m, 2H), 1.62-1.52 (m, 4H), 1.45 (m, 4H), 1.32-1.23 (m, 43H), 0.88 (t, J=6.8 Hz, 12H)

### Series 900-2 Characterization Data

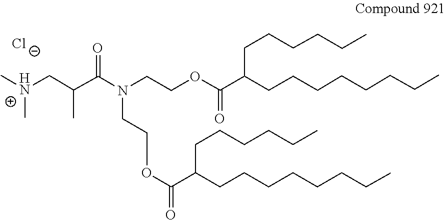

LCMS [M+1]⁺=695.6. ¹H NMR (400 MHz, chloroform-d) δ=12.55 (m, 1H), 4.38 (br t, J=6.0 Hz, 2H), 4.27-4.14 (m, 2H), 4.02-3.89 (m, 1H), 3.84-3.61 (m, 4H), 3.61-3.50 (m, 1H), 2.96 (br d, J=11.6 Hz, 1H), 2.83 (br d, J=3.6 Hz, 3H), 2.69 (br d, J=4.0 Hz, 3H), 2.39-2.25 (m, 2H), 1.58 (m, 4H), 1.46 (m, 4H), 1.26 (br s, 43H), 0.88 (t, J=6.8 Hz, 12H).

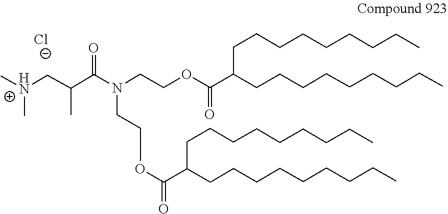

LCMS [M+1]⁺=807.8. ¹H NMR (400 MHz, chloroform-d) δ=12.73-12.49 (m, 1H), 4.38 (br t, J=5.2 Hz, 2H), 4.27-4.14 (m, 2H), 4.01-3.87 (m, 1H), 3.84-3.61 (m, 4H), 3.60-3.49 (m, 1H), 3.02-2.90 (m, 1H), 2.82 (br d, J=2.8 Hz, 3H), 2.69 (br d, J=3.2 Hz, 3H), 2.39-2.26 (m, 2H), 1.63-1.51 (m, 4H), 1.50-1.40 (m, 4H), 1.34-1.16 (m, 56H), 0.88 (t, J=6.8 Hz, 12H).

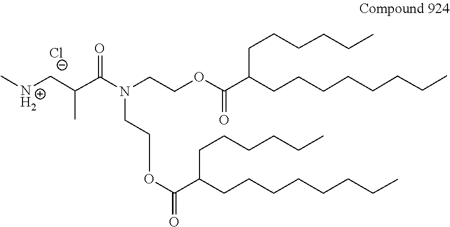 [SMILES: CCCCCCCCC(CCCCCC)C(=O)OCCN(CCOC(=O)C(CCCCCC)CCCCCCCC)C(=O)C(C)C[NH2+]C ; [Cl-]]

LCMS [M+1]⁺=681.6. ¹H NMR (400 MHz, chloroform-d) δ=10.89-10.00 (m, 1H), 8.36-7.63 (m, 1H), 4.61-4.06 (m, 4H), 3.96-3.22 (m, 6H), 3.19-2.95 (m, 1H), 2.79 (m, 3H), 2.49-2.23 (m, 2H), 1.58 (br s, 4H), 1.45 (m, 4H), 1.26 (br s, 43H), 0.89 (br t, J=6.4 Hz, 12H)

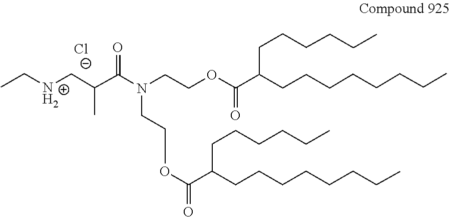 [SMILES: CCCCCCCCC(CCCCCC)C(=O)OCCN(CCOC(=O)C(CCCCCC)CCCCCCCC)C(=O)C(C)C[NH2+]CC ; [Cl-]]

LCMS [M+1]⁺=695.6. ¹H NMR (400 MHz, DMSO-d6) δ=8.68-8.03 (m, 2H), 4.27-4.17 (m, 2H), 4.16-4.03 (m, 2H), 3.78-3.57 (m, 3H), 3.44-3.34 (m, 2H), 3.11 (br d, J=9.6 Hz, 2H), 2.93 (br d, J=7.6 Hz, 2H), 2.35-2.22 (m, 2H), 1.55-1.33 (m, 8H), 1.30-1.14 (m, 43H), 1.11 (br d, J=6.4 Hz, 3H), 0.84 (t, J=6.4 Hz, 12H)

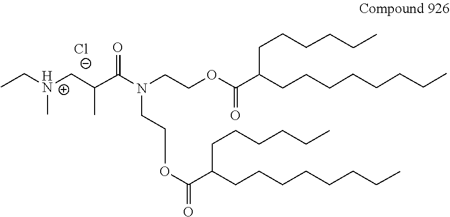

LCMS [M+1]⁺=709.6. ¹H NMR (400 MHz, DMSO-d₆) δ=9.37-9.36 (m, 1H), 9.56-8.96 (m, 1H), 4.30-4.16 (m, 2H), 4.16-4.02 (m, 2H), 3.81-3.60 (m, 3H), 3.57-3.33 (m, 2H), 3.20-2.89 (m, 3H), 2.76-2.61 (m, 3H), 2.36-2.19 (m, 2H), 1.57-1.32 (m, 8H), 1.22 (br s, 43H), 1.12-1.03 (m, 3H), 0.91-0.78 (m, 12H)

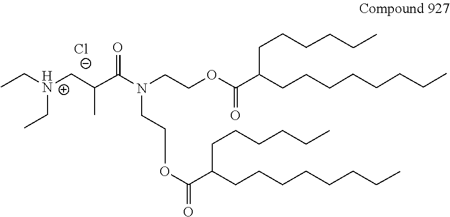

LCMS [M+1]⁺=723.8. ¹H NMR (CHLOROFORM-d, 400 MHz) δ=12.0-12.2 (m, 1H), 4.3-4.4 (m, 2H), 4.20 (q, 2H, J=5.2 Hz), 4.0-4.1 (m, 1H), 3.88 (br t, 1H, J=8.4 Hz), 3.5-3.8 (m, 4H), 3.0-3.3 (m, 3H), 2.8-3.0 (m, 2H), 2.3-2.4 (m, 2H), 1.57 (br dd, 4H, J=5.2, 7.6 Hz), 1.4-1.5 (m, 7H), 1.2-1.4 (m, 46H), 0.88 (t, 12H, J=6.4 Hz)

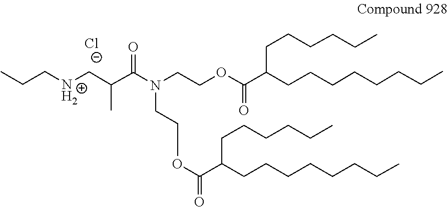 [SMILES: CCCCCCCCC(CCCCCC)C(=O)OCCN(CCOC(=O)C(CCCCCC)CCCCCCCC)C(=O)C(C)C[NH2+]CCC ; [Cl-]]

LCMS [M+1]⁺=709.6. ¹H NMR (400 MHz, chloroform-d) δ=11.15-10.74 (m, 1H), 7.26-7.18 (m, 1H), 4.60-4.46 (m, 1H), 4.44-4.32 (m, 1H), 4.29-4.16 (m, 1H), 4.14-4.06 (m, 1H), 3.85-3.76 (m, 2H), 3.69-3.54 (m, 2H), 3.49-3.36 (m, 1H), 3.34-3.24 (m, 1H), 3.15-2.85 (m, 3H), 2.40-2.25 (m, 2H), 1.99-1.83 (m, 2H), 1.58 (s, 4H), 1.50-1.40 (m, 4H), 1.26 (br s, 43H), 1.09-1.01 (m, 3H), 0.89 (t, J=6.8 Hz, 12H).

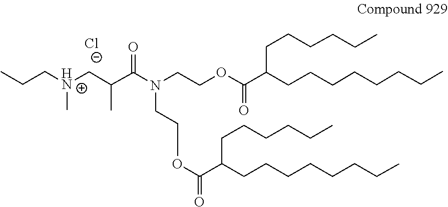

LCMS [M+1]⁺=737.7. ¹H NMR (400 MHz, chloroform-d) δ=12.46-11.83 (m, 1H), 4.45-4.32 (m, 2H), 4.31-4.12 (m, 2H), 4.11-3.96 (m, 1H), 3.88 (td, J=7.2, 14.4 Hz, 1H), 3.82-3.49 (m, 4H), 3.18-3.05 (m, 1H), 3.02-2.68 (m, 4H), 2.40-2.26 (m, 2H), 1.93-1.73 (m, 2H), 1.62-1.54 (m, 4H), 1.51-1.39 (m, 7H), 1.34-1.18 (m, 43H), 1.04-0.94 (m, 3H), 0.88 (t, J=6.8 Hz, 12H)

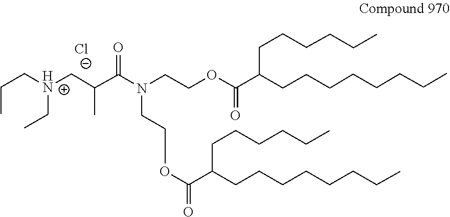

LCMS [M+1]⁺=751.7. ¹H NMR (400 MHz, chloroform-d) δ=11.99 (br s, 1H), 4.38 (br s, 2H), 4.31-4.21 (m, 1H), 4.20-4.10 (m, 1H), 4.07-3.94 (m, 1H), 3.92-3.83 (m, 1H), 3.76 (m, 3H), 3.58-3.47 (m, 1H), 3.02-2.70 (m, 5H), 2.33 (td, J=4.4, 8.8 Hz, 2H), 1.96-1.82 (m, 4H), 1.57 (m, 4H), 1.44 (m, 4H), 1.25 (br s, 43H), 1.04-0.92 (m, 6H), 0.88 (t, J=6.8 Hz, 12H)

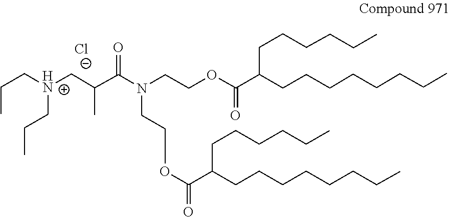

LCMS [M+1]⁺=751.7. ¹H NMR (400 MHz, chloroform-d) δ=11.99 (br s, 1H), 4.38 (br s, 2H), 4.31-4.21 (m, 1H), 4.20-4.10 (m, 1H), 4.07-3.94 (m, 1H), 3.92-3.83 (m, 1H), 3.76 (m, 3H), 3.58-3.47 (m, 1H), 3.02-2.70 (m, 5H), 2.33 (td, J=4.4, 8.8 Hz, 2H), 1.96-1.82 (m, 4H), 1.57 (m, 4H), 1.44 (m, 4H), 1.25 (br s, 43H), 1.04-0.92 (m, 6H), 0.88 (t, J=6.8 Hz, 12H)

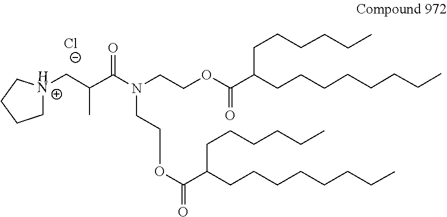

[M+1]⁺=721.7. ¹H NMR (400 MHz, chloroform-d) δ=12.46 (m, 1H), 4.37 (m, 2H), 4.21 (m, 3H), 3.91-3.73 (m, 2H), 3.73-3.53 (m, 4H), 3.53-3.40 (m, 1H), 3.11 (br d, J=12.4 Hz, 1H), 2.91-2.72 (m, 2H), 2.33 (br d, J=5.2 Hz, 2H), 2.19 (br d, J=5.2 Hz, 2H), 2.08-1.97 (m, 2H), 1.57 (m, 4H), 1.45 (m, 4H), 1.33-1.18 (m, 43H), 0.88 (t, J=6.8 Hz, 12H)

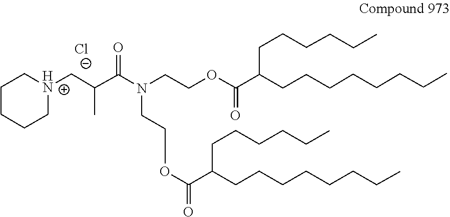

LCMS [M+1]⁺=735.8. ¹H NMR (400 MHz, chloroform-d) δ=12.39-11.74 (m, 1H), 4.43 (dd, J=5.6, 11.6 Hz, 1H), 4.37-4.25 (m, 1H), 4.19 (t, J=5.6 Hz, 2H), 4.09-3.84 (m, 2H), 3.79-3.51 (m, 4H), 3.45 (d, J=11.6 Hz, 1H), 3.28 (d, J=11.6 Hz, 1H), 2.87 (d, J=12.8 Hz, 1H), 2.77-2.51 (m, 2H), 2.42-2.07 (m, 4H), 1.82 (s, 3H), 1.61-1.51 (m, 4H), 1.50-1.38 (m, 5H), 1.35-1.14 (m, 43H), 0.88 (t, J=6.8 Hz, 12H)

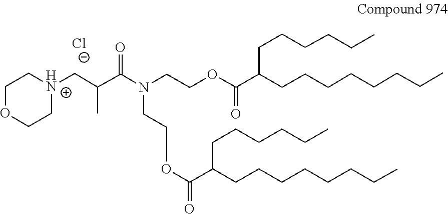

LCMS [M+1]⁺=737.6. 1H NMR (400 MHz, chloroform-d) δ=13.22-12.86 (m, 1H), 4.65-4.45 (m, 1H), 4.40-4.11 (m, 5H), 3.94 (m, 4H), 3.85-3.61 (m, 3H), 3.59-3.46 (m, 1H), 3.37 (br d, J=11.2 Hz, 1H), 3.27 (br d, J=12.4 Hz, 1H), 3.04-2.78 (m, 3H), 2.41-2.25 (m, 2H), 1.58 (m, 4H), 1.46 (br s, 4H), 1.26 (br s, 43H), 0.97-0.80 (m, 12H)

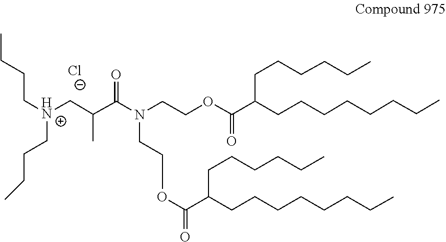

LCMS [M+1]⁺=779.7. ¹H NMR (400 MHz, chloroform-d) δ=12.18-11.91 (m, 1H), 4.38 (br t, J=5.6 Hz, 2H), 4.31-4.20 (m, 1H), 4.19-4.10 (m, 1H), 4.07-3.95 (m, 1H), 3.93-3.84 (m, 1H), 3.83-3.61 (m, 3H), 3.58-3.46 (m, 1H), 3.07-2.90 (m, 3H), 2.90-2.72 (m, 2H), 2.41-2.29 (m, 2H), 1.85-1.77 (m, 2H), 1.76-1.68 (m, 2H), 1.52-1.36 (m, 8H), 1.36-1.18 (m, 47H), 1.03-0.93 (m, 5H), 0.88 (t, J=6.8 Hz, 13H)

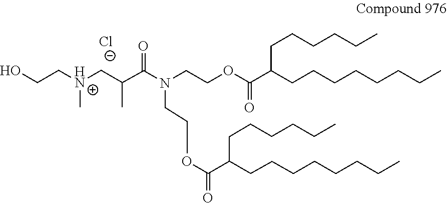

LCMS [M+1]⁺=725.6. ¹H NMR (400 MHz, DMSO-d₆) δ=9.56-8.95 (m, 1H), 5.40-5.23 (m, 1H), 4.30-4.03 (m, 4H), 3.83-3.53 (m, 6H), 3.47-3.39 (m, 2H), 3.21 (s, 3H), 2.82-2.66 (m, 3H), 2.35-2.23 (m, 2H), 1.56-1.45 (m, 4H), 1.41 (m, 4H), 1.22 (s, 40H), 1.09 (t, J=7.6 Hz, 3H), 0.85 (t, J=6.4 Hz, 12H)

### Series 900-3

### Representative Synthesis and Characterization Data for Compounds 931-939, 980-986

### Route:

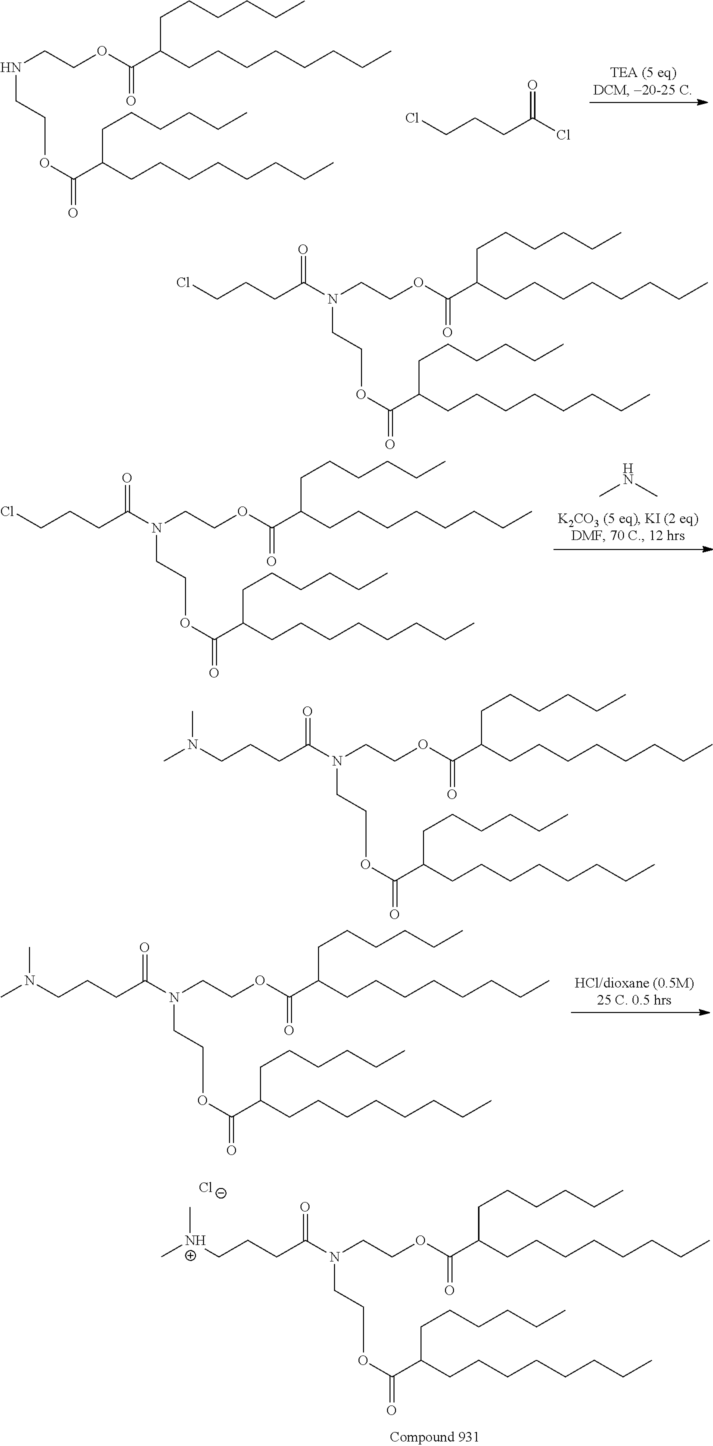

### General Synthetic Procedure for Preparation of Protected Lipidated Diethanolamine:

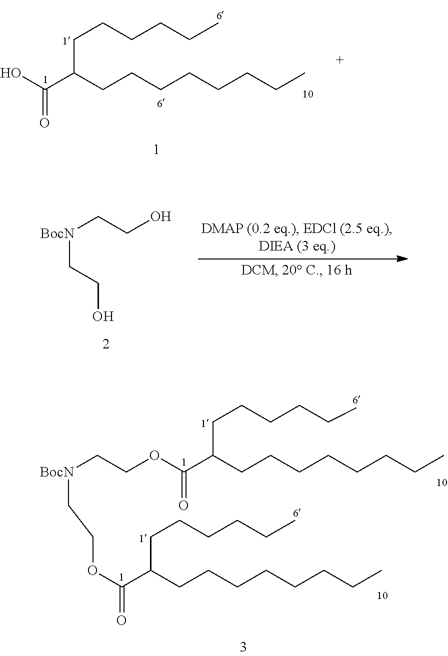 [SMILES: CCCCCCCCC(CCCCCC)C(=O)O ; CC(C)(C)OC(=O)N(CCO)CCO ; CCCCCCCCC(CCCCCC)C(=O)OCCN(CCOC(=O)C(CCCCCC)CCCCCCCC)C(=O)OC(C)(C)C]

To a solution of Compound 1 (13.74 g, 53.59 mmol, 2.2 eq.) in DCM (50 mL) was added DMAP (595.21 mg, 4.87 mmol, 0.2 eq.), EDCI (11.67 g, 60.90 mmol, 2.5 eq.), DIEA (9.45 g, 73.08 mmol, 12.73 mL, 3 eq.) and Compound 2 (5 g, 24.36 mmol, 4.61 mL, 1 eq.). The mixture was stirred at 20° C. for 16 hours. TLC (Petroleum ether:Ethyl acetate=3:1, P1:R_{f}=0.64) indicated Compound 1 and Compound 2 was consumed completely and new spots formed. The reaction mixture was quenched by addition sat. NaHCO₃ (10 mL) at 20° C., and then diluted with H₂O (40 mL) and extracted with DCM (80 mL). The combined organic layers were washed with brine (30 mL), dried over Na₂SO₄, filtered and concentrated under reduced pressure to give a residue. The residue was purified by column chromatography (SiO₂, Petroleum ether:Ethyl acetate=200:1 to 70:1 to 30:1) to give Compound 3 (11.48 g, 16.81 mmol, 52.13% yield, 99.9% purity) as colorless oil.

LCMS [M−100+1]⁺=582.5. ¹H NMR (400 MHz, chloroform-d) δ=4.33-4.07 (m, 4H), 3.66-3.31 (m, 4H), 2.54-2.18 (m, 2H), 1.64-1.55 (m, 4H), 1.50-1.38 (m, 13H), 1.34-1.19 (m, 40H), 0.88 (t, J=6.8 Hz, 12H)

### General Deprotection Procedure for Lipidated Diethanolamines: Procedure for Preparation of Compound 4

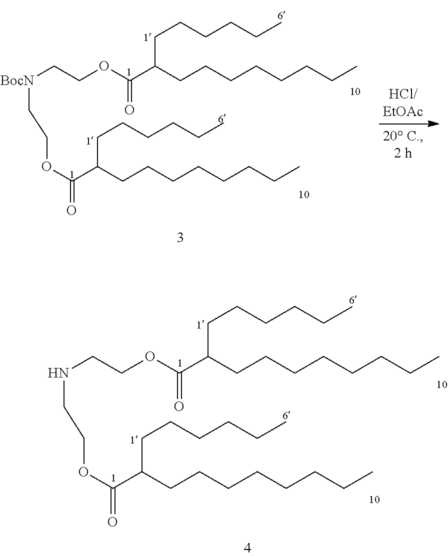 [SMILES: CCCCCCCCC(CCCCCC)C(=O)OCCN(CCOC(=O)C(CCCCCC)CCCCCCCC)C(=O)OC(C)(C)C ; CCCCCCCCC(CCCCCC)C(=O)OCCNCCOC(=O)C(CCCCCC)CCCCCCCC]

A solution of Compound 3 (9.48 g, 13.90 mmol, 1 eq.) in HCl/EtOAc (4 M, 47.40 mL, 13.64 eq.) was stirred at 20° C. for 2 hours. LC-MS showed Compound 3 was consumed and desired MS was detected. The reaction mixture was concentrated under reduced pressure to give Compound 4 (8.6 g, crude, HCl) as colorless oil.

LCMS [M+1]⁺=582.5. ¹H NMR (400 MHz, chloroform-d) δ=9.87 (br s, 2H), 4.51 (br s, 4H), 3.36 (br s, 4H), 2.52-2.31 (m, 2H), 1.71-1.56 (m, 4H), 1.53-1.41 (m, 4H), 1.38-1.18 (m, 40H), 0.88 (t, J=6.8 Hz, 12H)

### General Procedure for the Preparation of Common Intermediate Alkyl Halides: Preparation of Compound 6a):

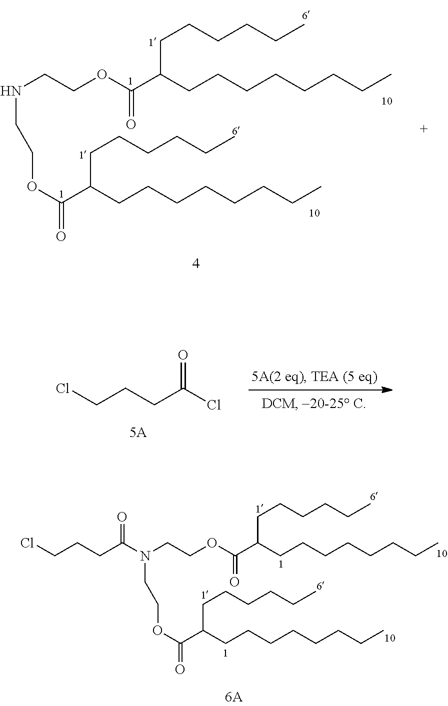 [SMILES: CCCCCCCCC(CCCCCC)C(=O)OCCNCCOC(=O)C(CCCCCC)CCCCCCCC ; O=C(Cl)CCCCl ; CCCCCCCCC(CCCCCC)C(=O)OCCN(CCOC(=O)C(CCCCCC)CCCCCCCC)C(=O)CCCCl]

To a solution of Compound 4 (10 g, 17.18 mmol, 1 eq., HCl salt) in DCM (100 mL) was added TEA (8.69 g, 85.92 mmol, 11.96 mL, 5 eq.) and Compound 5A (4.85 g, 34.37 mmol, 3.85 mL, 2 eq.) at −20° C. The reaction mixture was stirred at 25° C. for 12 hours. TLC (Petroleum ether:Ethyl acetate=5:1, R_{f}=0.56) indicated Compound 4 was consumed completely and new spots formed. The mixture was concentrated under reduced pressure to give a residue. The residue was purified by column chromatography (SiO₂, Petroleum ether:Ethyl acetate=92:8) to give Compound 6A (10 g, 14.57 mmol, 84.77% yield) as yellow oil.

¹H NMR (400 MHz, chloroform-d) δ=4.27-4.17 (m, 4H), 3.70-3.56 (m, 6H), 2.68-2.52 (m, 2H), 2.34 (br d, J=8.8 Hz, 2H), 2.14 (t, J=6.4 Hz, 2H), 1.62-1.56 (m, 4H), 1.51-1.41 (m, 4H), 1.26 (s, 37H), 0.89 (t, J=6.8 Hz, 12H)

### General Procedure for Amination of Common Intermediate 6a: Preparation of Compound 7A

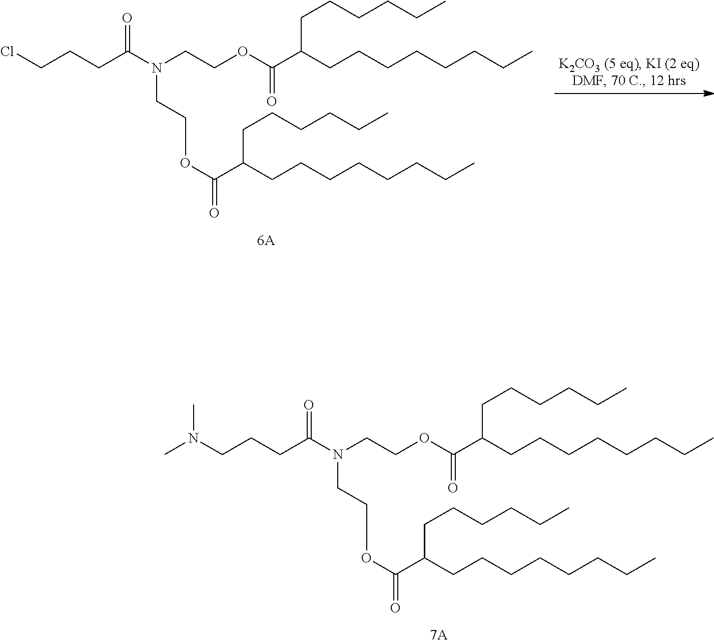

To a solution of Compound 6A (500 mg, 728.35 μmol, 1 eq.) in in DMF (3 mL) was added K₂CO₃ (503.31 mg, 3.64 mmol, 5 eq.), KI (241.81 mg, 1.46 mmol, 2 eq.) and N-methylmethanamine (2 M, 5.38 mL, 20 eq.). The reaction mixture was stirred at 70° C. for 12 hours. TLC (DCM:MeOH=10:1, R_{f}=0.56) indicated Compound 6A was consumed completely and new spots formed. The mixture was concentrated under reduced pressure to give a residue. The residue was purified by column chromatography (SiO₂, Petroleum ether:Ethyl acetate=4:1 to DCM:MeOH=10:1) to give Compound 7A (310 mg, 445.97 μmol, 61.23% yield) as yellow oil. LCMS [M+1]⁺=695.6.

### General Procedure for HCl Salt Formation: Preparation of Compound 931

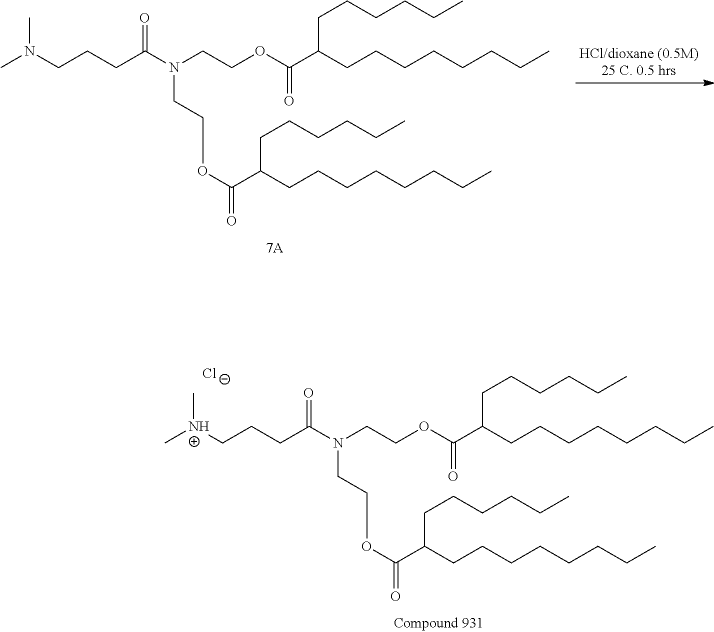

To a solution of Compound 7A (0.108 g, 155.37 μmol, 1 eq) in DCM (1 mL) was added HCl/dioxane (1.5 mL, 1 M) at 25° C. The reaction mixture was stirred at 25° C. for 1 hour. The reaction mixture was concentrated and lyophilized to give Compound 931 (105 mg, 151.06 μmol, 97.22% yield, HCl salt) as yellow gum.

LCMS [M+1]⁺=695.6. ¹H NMR (400 MHz, chloroform-d) δ=12.32 (br s, 1H), 4.21 (t, J=5.2 Hz, 4H), 3.67-3.55 (m, 4H), 3.13 (br s, 2H), 2.82 (d, J=4.4 Hz, 6H), 2.64 (br s, 2H), 2.32 (dt, J=4.8, 8.4 Hz, 2H), 2.20 (br s, 2H), 1.56 (m, 4H), 1.49-1.41 (m, 4H), 1.34-1.21 (m, 40H), 0.88 (t, J=6.8 Hz, 12H)

### Series Characterization Data: Characterization of Compounds 931-39, 980-986

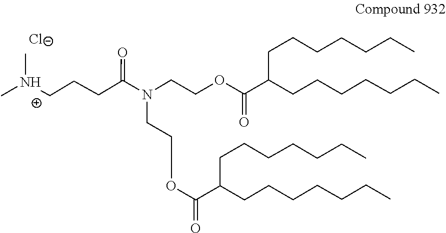

LCMS [M+1]⁺=695.7. ¹H NMR (400 MHz, chloroform-d) δ=12.39 (br s, 1H), 4.21 (t, J=5.2 Hz, 4H), 3.67-3.55 (m, 4H), 3.13 (br s, 2H), 2.82 (d, J=4.8 Hz, 6H), 2.65 (t, J=5.2 Hz, 2H), 2.32 (dd, J=3.6, 8.4 Hz, 2H), 2.20 (br s, 2H), 1.61-1.53 (m, 4H), 1.49-1.41 (m, 4H), 1.33-1.22 (m, 40H), 0.88 (t, J=6.8 Hz, 12H)

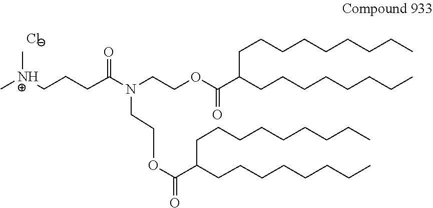 [SMILES: [Cl-] ; CCCCCCCCCC(CCCCCCCC)C(=O)OCCN(CCOC(=O)C(CCCCCCCC)CCCCCCCCC)C(=O)CCC[NH+](C)C]

LCMS [M+1]⁺=807.8. ¹H NMR (400 MHz, DMSO-d₆) δ=9.51-9.31 (m, 1H), 4.23-4.05 (m, 4H), 3.64-3.47 (m, 4H), 3.07-2.98 (m, 2H), 2.76 (s, 6H), 2.47 (s, 2H), 2.35-2.23 (m, 2H), 1.90-1.76 (m, 2H), 1.54-1.44 (m, 4H), 1.43-1.35 (m, 4H), 1.23 (s, 56H), 0.85 (t, J=6.8 Hz, 12H)

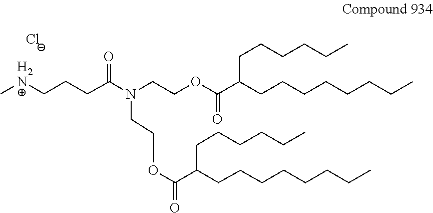

LCMS [M+1]⁺=681.6. ¹H NMR (400 MHz, chloroform-d) δ=9.77 (m, 2H), 4.22 (br d, J=3.2 Hz, 4H), 3.61 (br d, J=5.6 Hz, 4H), 3.10 (br s, 2H), 2.73 (br s, 5H), 2.40-2.27 (m, 1H), 2.15 (br s, 2H), 1.59-1.52 (m, 4H), 1.50-1.42 (m, 4H), 1.26 (br s, 40H), 0.89 (br t, J=6.4 Hz, 12H)

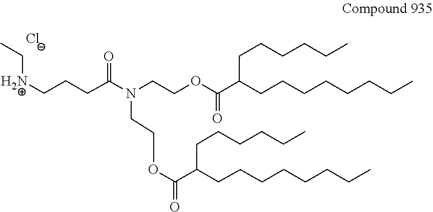 [SMILES: [Cl-] ; CCCCCCCCC(CCCCCC)C(=O)OCCN(CCOC(=O)C(CCCCCC)CCCCCCCC)C(=O)CCC[NH2+]CC]

LCMS [M+1]⁺=695.6. ¹H NMR (400 MHz, chloroform-d) δ=9.38 (br s, 2H), 4.23 (br s, 4H), 3.68-3.54 (m, 4H), 3.13 (d, J=1.6 Hz, 4H), 2.75 (br s, 2H), 2.36-2.30 (m, 2H), 2.17 (br s, 2H), 1.62-1.54 (m, 4H), 1.47 (br s, 7H), 1.26 (br s, 41H), 0.88 (t, J=6.8 Hz, 12H)

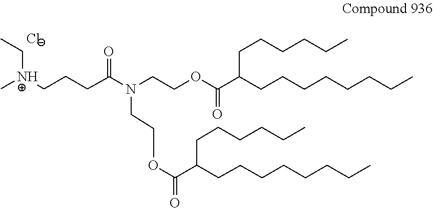 [SMILES: [Cl-] ; CCCCCCCCC(CCCCCC)C(=O)OCCN(CCOC(=O)C(CCCCCC)CCCCCCCC)C(=O)CCC[NH+](C)CC]

LCMS [M+1]⁺=709.6. ¹H NMR (400 MHz, chloroform-d) δ=12.03-11.85 (m, 1H), 4.21 (br s, 4H), 3.61 (td, J=5.6, 11.2 Hz, 4H), 3.30-3.01 (m, 4H), 2.79 (d, J=4.8 Hz, 3H), 2.65 (br s, 2H), 2.41-2.26 (m, 2H), 2.23-2.11 (m, 2H), 1.50-1.41 (m, 8H), 1.33-1.21 (m, 43H), 0.88 (t, J=6.8 Hz, 12H)

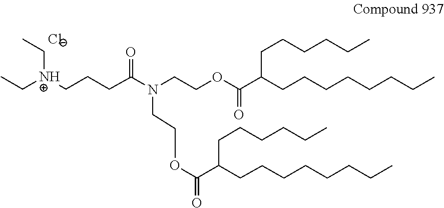 [SMILES: [Cl-] ; CCCCCCCCC(CCCCCC)C(=O)OCCN(CCOC(=O)C(CCCCCC)CCCCCCCC)C(=O)CCC[NH+](CC)CC]

LCMS [M+1]⁺=723.6. ¹H NMR (400 MHz, chloroform-d) δ=11.57 (m, 1H), 4.29-4.12 (m, 4H), 3.69-3.54 (m, 4H), 3.16 (br s, 6H), 2.64 (br s, 2H), 2.32 (dq, J=5.6, 8.4 Hz, 2H), 2.18 (br s, 2H), 1.60-1.53 (m, 4H), 1.45 (br t, J=6.4 Hz, 10H), 1.32-1.19 (m, 40H), 0.88 (t, J=6.8 Hz, 12H)

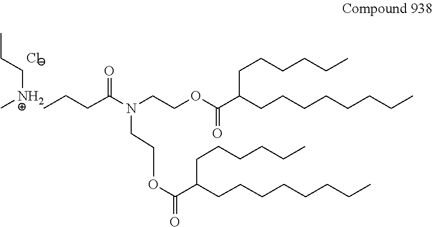 [SMILES: CCCCCCCCC(CCCCCC)C(=O)OCCN(CCOC(=O)C(CCCCCC)CCCCCCCC)C(=O)CCC[NH+](C)CCC ; [Cl-]]

LCMS [M+1]⁺=709.6. ¹H NMR (400 MHz, DMSO-d₆) δ=8.56 (br s, 2H), 4.17 (s, 2H), 4.09 (s, 2H), 3.59 (br s, 2H), 3.51 (s, 2H), 2.94-2.77 (m, 4H), 2.48 (br s, 2H), 2.29 (br dd, J=3.2, 8.4 Hz, 2H), 1.87-1.76 (m, 2H), 1.63-1.55 (m, 2H), 1.50-1.35 (m, 8H), 1.31-1.15 (m, 40H), 0.91 (t, J=7.6 Hz, 3H), 0.85 (br t, J=6.4 Hz, 12H)

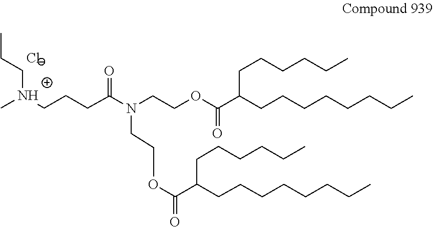 [SMILES: CCCCCCCCC(CCCCCC)C(=O)OCCN(CCOC(=O)C(CCCCCC)CCCCCCCC)C(=O)CCC[NH+](C)CCC ; [Cl-]]

LCMS [M+1]⁺=723.6. ¹H NMR (400 MHz, chloroform-d) δ=10.73 (br s, 1H), 4.22 (br s, 4H), 3.69-3.54 (m, 4H), 3.28-3.11 (m, 3H), 3.01-2.94 (m, 1H), 2.82 (br d, J=4.4 Hz, 3H), 2.69 (br s, 2H), 2.37-2.29 (m, 2H), 2.22 (br s, 2H), 1.99-1.87 (m, 2H), 1.62-1.53 (m, 4H), 1.50-1.42 (m, 4H), 1.32-1.20 (m, 40H), 1.05 (t, J=7.2 Hz, 3H), 0.89 (t, J=6.8 Hz, 12H)

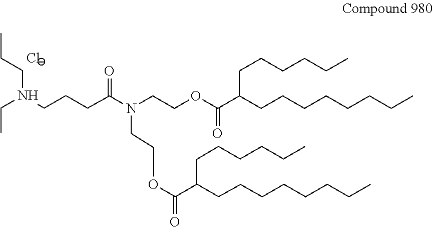 [SMILES: CCCCCCCCC(CCCCCC)C(=O)OCCN(CCOC(=O)C(CCCCCC)CCCCCCCC)C(=O)CCCN(CC)CCC ; [Cl-]]

LCMS [M+1]⁺=737.6. ¹H NMR (400 MHz, DMSO-d₆) δ=9.54-9.25 (m, 1H), 4.17 (br t, J=5.6 Hz, 2H), 4.09 (br t, J=5.6 Hz, 2H), 3.70-3.55 (m, 2H), 3.54-3.40 (m, 2H), 3.18-3.09 (m, 2H), 3.07-2.94 (m, 4H), 2.33-2.23 (m, 2H), 1.88-1.77 (m, 2H), 1.70 (br s, 2H), 1.47 (m, 4H), 1.43-1.35 (m, 4H), 1.32-1.12 (m, 45H), 0.92 (t, J=7.2 Hz, 3H), 0.85 (br t, J=6.4 Hz, 12H)

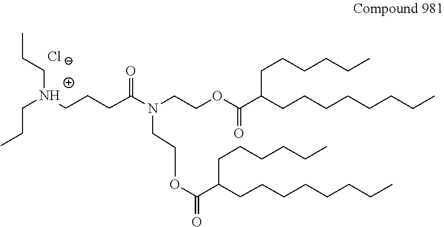 [SMILES: CCCCCCCCC(CCCCCC)C(=O)OCCN(CCOC(=O)C(CCCCCC)CCCCCCCC)C(=O)CCC[NH+](CCC)CCC ; [Cl-]]

LCMS [M+1]⁺=751.7. ¹H NMR (400 MHz, chloroform-d) δ=11.84 (m, 1H), 4.35-4.07 (m, 4H), 3.69-3.52 (m, 4H), 3.13 (br s, 2H), 2.99 (m, 4H), 2.67-2.55 (m, 2H), 2.32 (dq, J=5.6, 8.8 Hz, 2H), 2.18 (m, 2H), 1.89 (br s, 4H), 1.63-1.52 (m, 4H), 1.49-1.41 (m, 4H), 1.33-1.18 (m, 40H), 1.02 (br t, J=7.2 Hz, 6H), 0.88 (t, J=6.8 Hz, 12H)

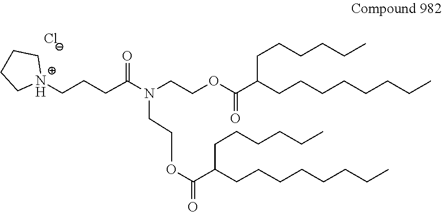 [SMILES: [Cl-] ; CCCCCCCCC(CCCCCC)C(=O)OCCN(CCOC(=O)C(CCCCCC)CCCCCCCC)C(=O)CCC[NH+]1CCCC1]

LCMS [M+1]⁺=721.6. ¹H NMR (400 MHz, chloroform-d) δ=10.82-10.69 (m, 1H), 4.24-4.17 (m, 4H), 3.86 (br s, 2H), 3.61 (td, J=5.6, 17.2 Hz, 4H), 3.30-3.20 (m, 2H), 2.91 (br s, 2H), 2.67 (br t, J=6.0 Hz, 2H), 2.31 (td, J=4.4, 8.8 Hz, 2H), 2.23 (br s, 4H), 2.12 (br s, 2H), 1.61-1.52 (m, 4H), 1.49-1.41 (m, 4H), 1.25 (s, 36H), 0.92-0.84 (m, 12H)

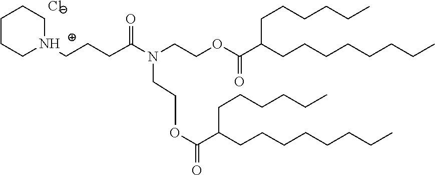 [SMILES: [Cl-] ; CCCCCCCCC(CCCCCC)C(=O)OCCN(CCOC(=O)C(CCCCCC)CCCCCCCC)C(=O)CCC[NH+]1CCCCC1]

LCMS [M+1]⁺=735.7. ¹H NMR (400 MHz, chloroform-d) δ=11.86 (br s, 1H), 4.20 (br d, J=3.2 Hz, 4H), 3.68-3.51 (m, 6H), 3.06 (br s, 2H), 2.63 (m, 4H), 2.37-2.21 (m, 6H), 1.88 (br d, J=0.9 Hz, 4H), 1.56 (br dd, J=6.0, 13.6 Hz, 4H), 1.47-1.41 (m, 4H), 1.32-1.21 (m, 40H), 0.88 (t, J=6.8 Hz, 12H)

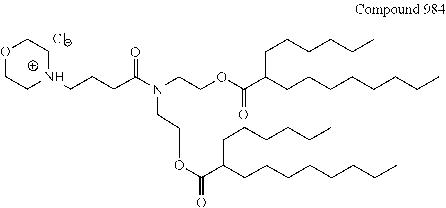 [SMILES: [Cl-] ; CCCCCCCCC(CCCCCC)C(=O)OCCN(CCOC(=O)C(CCCCCC)CCCCCCCC)C(=O)CCC[NH+]1CCOCC1]

LCMS [M+1]⁺=737.6. 1H NMR (400 MHz, chloroform-d) δ=12.99-12.81 (m, 1H), 4.31 (br t, J=12.0 Hz, 2H), 4.20 (q, J=5.6 Hz, 4H), 3.98 (dd, J=2.8, 12.8 Hz, 2H), 3.66-3.55 (m, 4H), 3.48 (br d, J=12.0 Hz, 2H), 3.15-3.07 (m, 2H), 2.97-2.84 (m, 2H), 2.64 (br t, J=5.6 Hz, 2H), 2.38-2.20 (m, 4H), 1.56 (br dd, J=6.0, 14.0 Hz, 4H), 1.50-1.40 (m, 4H), 1.35-1.16 (m, 40H), 0.89 (t, J=6.8 Hz, 12H)

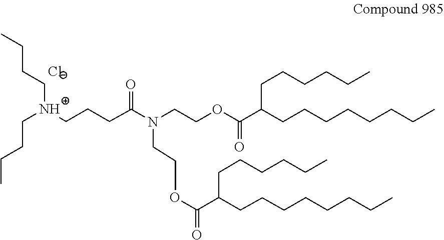 [SMILES: CCCCCCCCC(CCCCCC)C(=O)OCCN(CCOC(=O)C(CCCCCC)CCCCCCCC)C(=O)CCC[NH+](CCCC)CCCC ; [Cl-]]

LCMS [M+1]⁺=779.7. ¹H NMR (400 MHz, DMSO-d₆) δ=9.71-9.45 (m, 1H), 4.23-4.13 (m, 2H), 4.12-4.04 (m, 2H), 3.64-3.56 (m, 2H), 3.56-3.47 (m, 2H), 3.12-2.95 (m, 6H), 2.35-2.23 (m, 2H), 1.91-1.77 (m, 2H), 1.62 (td, J=8.0, 15.2 Hz, 4H), 1.52-1.37 (m, 8H), 1.32 (br dd, J=7.2, 14.8 Hz, 6H), 1.27-1.14 (m, 40H), 0.91 (t, J=7.2 Hz, 6H), 0.85 (br t, J=6.4 Hz, 12H)

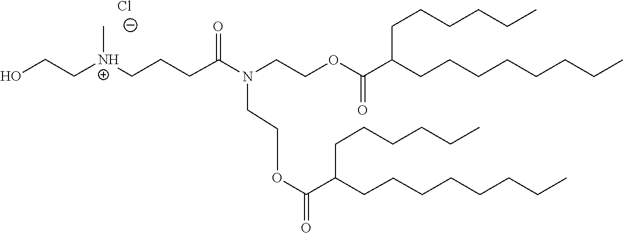 [SMILES: [Cl-] ; CCCCCCCCC(CCCCCC)C(=O)OCCN(CCOC(=O)C(CCCCCC)CCCCCCCC)C(=O)CCC[NH+](C)CCO]

LCMS [M+1]⁺=725.6. 1H NMR (400 MHz, DMSO-d6) δ=9.79-9.41 (m, 1H), 5.32 (br s, 1H), 4.17 (br t, J=5.6 Hz, 2H), 4.09 (t, J=5.6 Hz, 2H), 3.73 (q, J=4.8 Hz, 2H), 3.59 (br t, J=5.6 Hz, 2H), 3.51 (br t, J=5.6 Hz, 2H), 3.26-2.97 (m, 4H), 2.77 (s, 3H), 2.49-2.42 (m, 2H), 2.29 (dt, J=5.4, 8.4 Hz, 2H), 1.94-1.77 (m, 2H), 1.56-1.44 (m, 4H), 1.43-1.34 (m, 4H), 1.22 (br s, 40H), 0.85 (t, J=6.8 Hz, 12H)

### Series 900-4

### Representative Synthesis and Characterization Data for Compounds 941-949, 990-996

### Route:

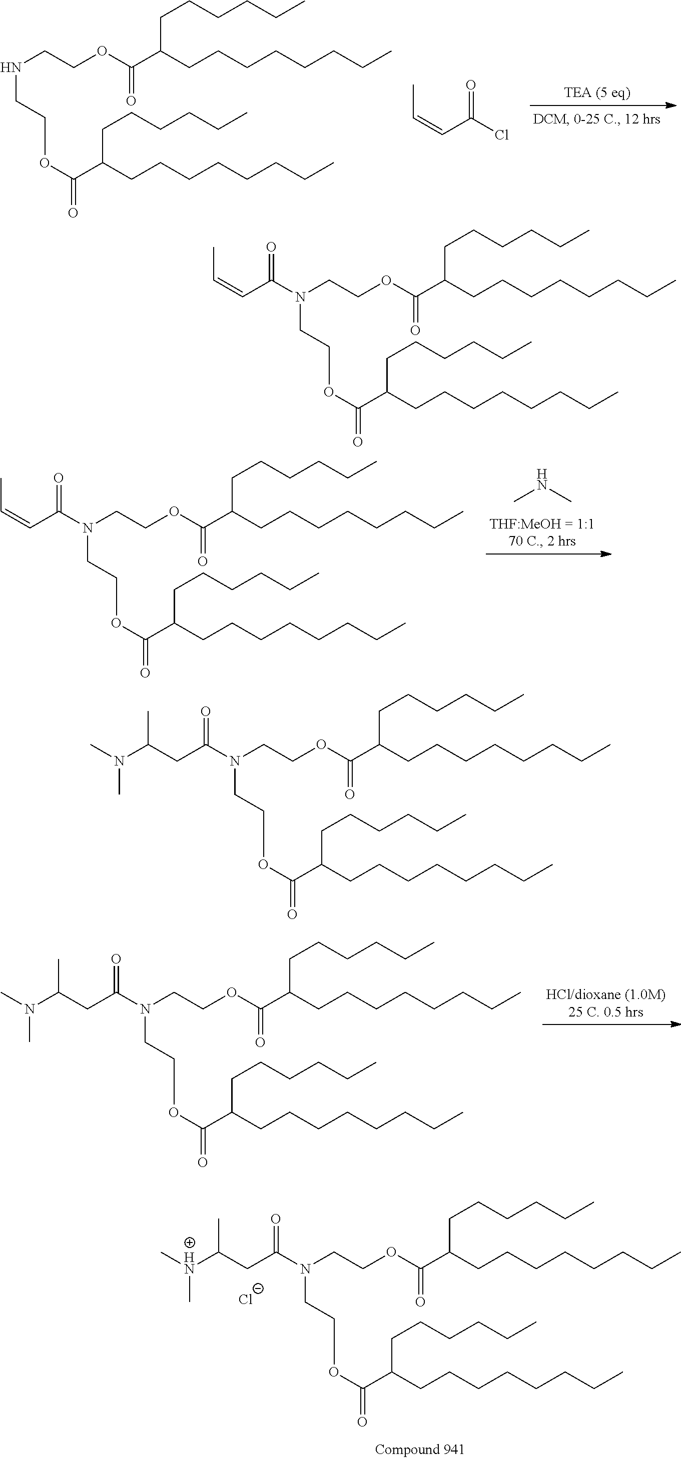

### General Synthetic Procedure for Preparation of Protected Lipidated Diethanolamine: Synthesis of Compound 3

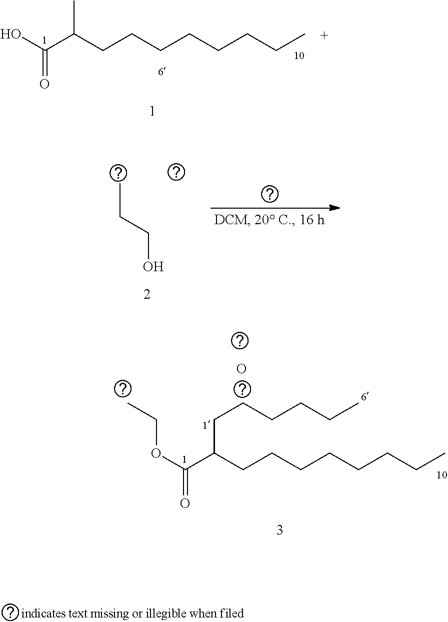

To a solution of Compound 1 (13.74 g, 53.59 mmol, 2.2 eq.) in DCM (50 mL) was added DMAP (595.21 mg, 4.87 mmol, 0.2 eq.), EDCI (11.67 g, 60.90 mmol, 2.5 eq.), DIEA (9.45 g, 73.08 mmol, 12.73 mL, 3 eq.) and Compound 2 (5 g, 24.36 mmol, 4.61 mL, 1 eq.). The mixture was stirred at 20° C. for 16 hours. TLC (Petroleum ether:Ethyl acetate=3:1, P1:R_{f}=0.64) indicated Compound 1 and Compound 2 was consumed completely and new spots formed. The reaction mixture was quenched by addition sat. NaHCO₃ (10 mL) at 20° C., and then diluted with H₂O (40 mL) and extracted with DCM (80 mL). The combined organic layers were washed with brine (30 mL), dried over Na₂SO₄, filtered and concentrated under reduced pressure to give a residue. The residue was purified by column chromatography (SiO₂, Petroleum ether:Ethyl acetate=200:1 to 70:1 to 30:1) to give Compound 3 (11.48 g, 16.81 mmol, 52.13% yield, 99.9% purity) as colorless oil.

LCMS [M−100+1]⁺=582.5. ¹H NMR (400 MHz, chloroform-d) δ=4.33-4.07 (m, 4H), 3.66-3.31 (m, 4H), 2.54-2.18 (m, 2H), 1.64-1.55 (m, 4H), 1.50-1.38 (m, 13H), 1.34-1.19 (m, 40H), 0.88 (t, J=6.8 Hz, 12H)

### General Deprotection Procedure for Lipidated Diethanolamines: Procedure for Preparation of Compound 4

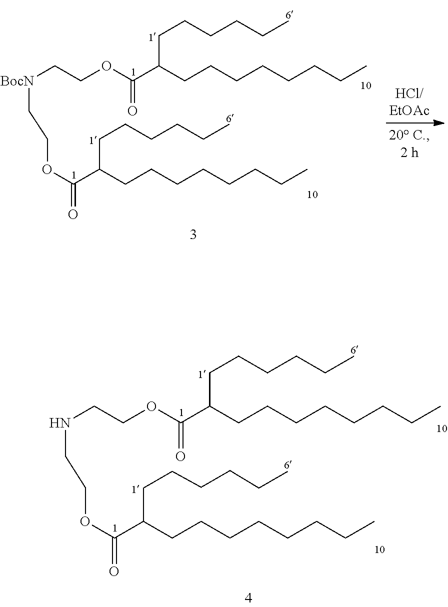 [SMILES: CCCCCCCCC(CCCCCC)C(=O)OCCN(CCOC(=O)C(CCCCCC)CCCCCCCC)C(=O)OC(C)(C)C ; CCCCCCCCC(CCCCCC)C(=O)OCCNCCOC(=O)C(CCCCCC)CCCCCCCC]

A solution of Compound 3 (9.48 g, 13.90 mmol, 1 eq.) in HCl/EtOAc (4 M, 47.40 mL, 13.64 eq.) was stirred at 20° C. for 2 hours. LC-MS showed Compound 3 was consumed and desired MS was detected. The reaction mixture was concentrated under reduced pressure to give Compound 4 (8.6 g, crude, HCl) as colorless oil.

LCMS [M+1]⁺=582.5. ¹H NMR (400 MHz, chloroform-d) δ=9.87 (br s, 2H), 4.51 (br s, 4H), 3.36 (br s, 4H), 2.52-2.31 (m, 2H), 1.71-1.56 (m, 4H), 1.53-1.41 (m, 4H), 1.38-1.18 (m, 40H), 0.88 (t, J=6.8 Hz, 12H)

### General Procedure for the Preparation of Common Intermediate Methacrylates: Preparation of Compound 6):

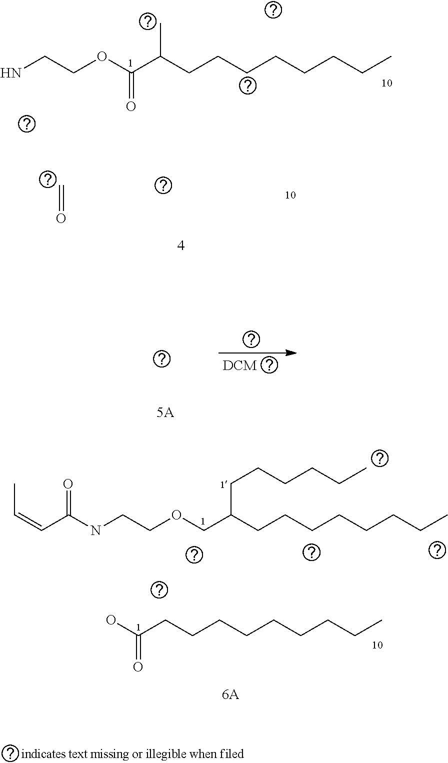

To a solution of Compound 4 (8 g, 13.75 mmol, 1 eq.) in DCM (50 mL) was added TEA (6.96 g, 68.73 mmol, 9.57 mL, 5 eq.) and Compound 5A (2.16 g, 20.62 mmol, 1.5 eq.) at 0° C. The reaction mixture was stirred at 25° C. for 12 hours. TLC (Petroleum ether:Ethyl acetate=5:1, R_{f}=0.56) indicated Compound 4 was consumed completely and new spots formed. The mixture was concentrated under reduced pressure to give a residue. The residue was purified by column chromatography (SiO₂, Petroleum ether:Ethyl acetate=92:8) to give Compound 6A (5.34 g, 8.22 mmol, 59.76% yield) as yellow oil.

¹H NMR (400 MHz, chloroform-d) δ=6.96 (dd, J=6.8, 14.8 Hz, 1H), 6.32 (dd, J=1.6, 14.8 Hz, 1H), 5.22-5.08 (m, 1H), 4.30-4.16 (m, 4H), 3.72-3.58 (m, 4H), 2.32 (tt, J=5.6, 8.8 Hz, 2H), 1.91 (dd, J=1.6, 6.8 Hz, 2H), 1.60-1.53 (m, 4H), 1.44 (m, 4H), 1.26 (br s, 40H), 0.88 (t, J=6.8 Hz, 12H)

### General Procedure for Amination of Common Intermediate 6a: Preparation of Compound 7A

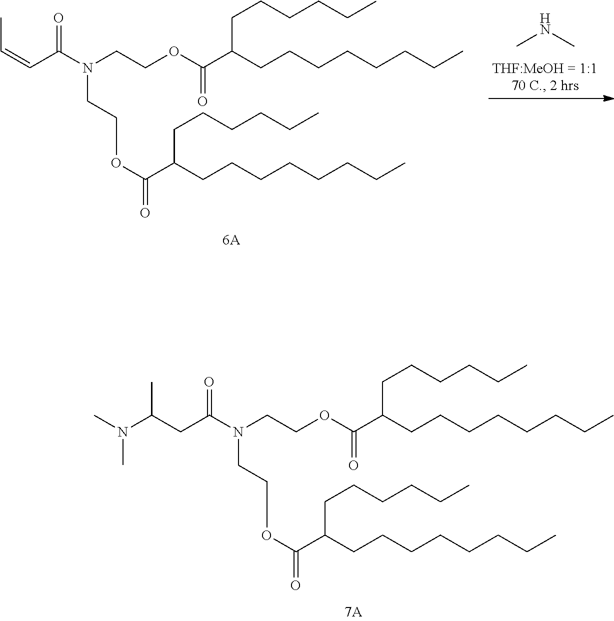

To a solution of Compound 6A (350 mg, 538.44 μmol, 1 eq.) in THF (1 mL) and MeOH (1 mL) was added dimethylamine (2 M, 5.38 mL, 20 eq.). The reaction mixture was stirred at 70° C. for 12 hours. TLC (Dichloromethane:Methanol=10:1, R_{f}=0.45) indicated Compound 6A was consumed completely and new spots formed. The mixture was concentrated under reduced pressure to give a residue. The residue was purified by column chromatography (SiO₂, Dichloromethane:Methanol=100:1 to 30:1) to give Compound 7A (320 mg, 460.36 μmol, 85.50% yield) as yellow oil. LCMS [M+1]⁺=695.6 General procedure for HCl salt formation: Preparation of Compound 941

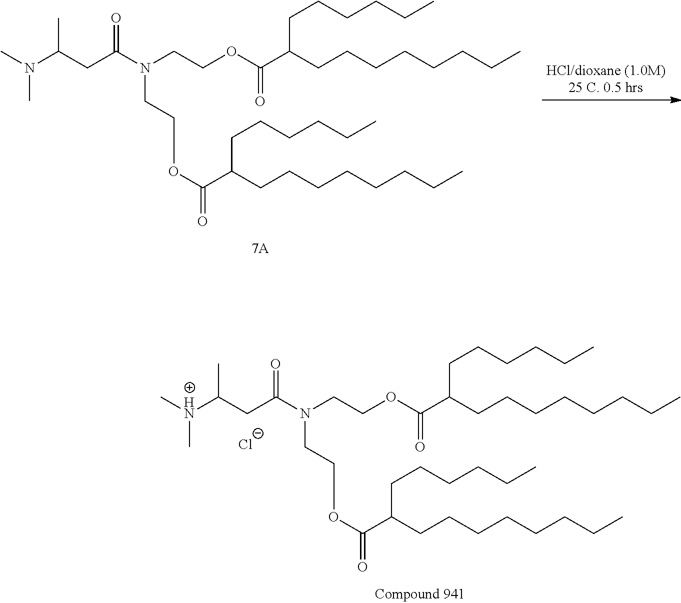

To a solution of Compound 7A (210 mg, 302.11 μmol, 1 eq.) in DCM (1 mL) was added HCl/dioxane (1 M, 1 mL) at 25° C. The reaction mixture was stirred at 25° C. for 0.5 hour. The reaction mixture was concentrated and lyophilized to give Compound 941 (210 mg, 287.05 μmol, 95.02% yield, HCl) as yellow oil. LCMS [M+1]⁺=695.6 ¹H NMR (400 MHz, chloroform-d) δ=12.57-12.34 (m, 1H), 4.30-4.11 (m, 4H), 3.95-3.83 (m, 1H), 3.80-3.70 (m, 1H), 3.67-3.41 (m, 4H), 2.80 (d, J=4.8 Hz, 3H), 2.74 (br d, J=4.8 Hz, 3H), 2.67 (br dd, J=8.8, 16.0 Hz, 1H), 2.38-2.27 (m, 2H), 1.63-1.52 (m, 4H), 1.49-1.40 (m, 7H), 1.31-1.17 (m, 40H), 0.96-0.78 (m, 12H)

### Series Characterization Data: Characterization of Compounds 942-949, 990-996

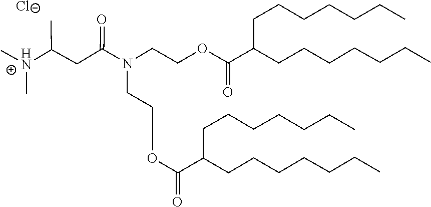

LCMS [M+1]⁺=695.7. ¹H NMR (400 MHz, chloroform-d) δ=12.52-12.28 (m, 1H), 4.29-4.12 (m, 4H), 3.94-3.80 (m, 1H), 3.79-3.68 (m, 1H), 3.65-3.39 (m, 4H), 2.86 (br d, J=4.8 Hz, 3H), 2.74 (br d, J=4.8 Hz, 3H), 2.70-2.61 (m, 1H), 2.33-2.28 (m, 2H), 1.62-1.51 (m, 4H), 1.48-1.40 (m, 7H), 1.31-1.21 (m, 40H), 0.88 (t, J=6.8 Hz, 12H)

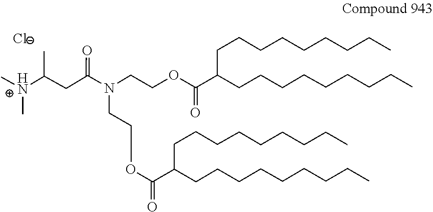

LCMS [M+1]⁺=807.8. ¹H NMR (400 MHz, chloroform-d) δ=12.40 (br s, 1H), 4.27-4.15 (m, 4H), 3.89 (d, J=2.0 Hz, 1H), 3.80-3.70 (m, 1H), 3.66-3.40 (m, 4H), 2.80 (d, J=4.8 Hz, 3H), 2.74 (d, J=4.8 Hz, 3H), 2.71-2.62 (m, 1H), 2.37-2.25 (m, 2H), 1.61-1.51 (m, 4H), 1.44 (q, J=6.8 Hz, 7H), 1.25 (s, 56H), 0.88 (t, J=6.8 Hz, 12H)

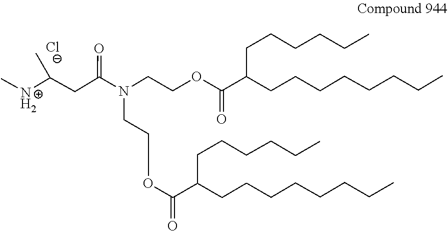 [SMILES: CCCCCCCCC(CCCCCC)C(=O)OCCN(CCOC(=O)C(CCCCCC)CCCCCCCC)C(=O)CC(C)[NH2+]C ; [Cl-]]

LCMS [M+1]⁺=681.6. ¹H NMR (400 MHz, chloroform-d) δ=10.46-10.22 (m, 1H), 9.08-8.80 (m, 1H), 4.32-4.13 (m, 4H), 3.75-3.48 (m, 5H), 3.22-3.08 (m, 1H), 2.99-2.85 (m, 1H), 2.74 (br s, 3H), 2.38-2.27 (m, 2H), 1.64-1.51 (m, 7H), 1.49-1.41 (m, 4H), 1.31-1.18 (m, 40H), 0.88 (t, J=6.8 Hz, 12H)

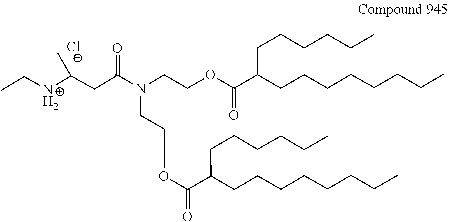 [SMILES: CCCCCCCCC(CCCCCC)C(=O)OCCN(CCOC(=O)C(CCCCCC)CCCCCCCC)C(=O)CC(C)[NH2+]CC ; [Cl-]]

LCMS [M+1]⁺=695.6. ¹H NMR (400 MHz, chloroform-d) δ=10.67-10.49 (m, 1H), 8.44 (br s, 1H), 4.22 (br t, J=5.6 Hz, 4H), 3.75-3.47 (m, 5H), 3.37-3.11 (m, 1H), 3.09-2.83 (m, 2H), 2.38-2.27 (m, 2H), 2.13 (br s, 3H), 1.63-1.55 (m, 4H), 1.51-1.41 (m, 7H), 1.26 (br s, 40H), 0.88 (t, J=6.8 Hz, 12H)

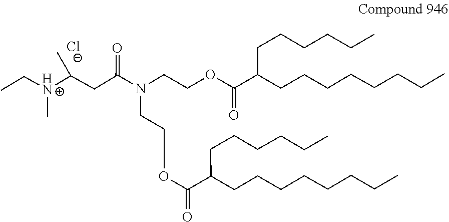

LCMS [M+1]⁺=709.6. ¹H NMR (400 MHz, chloroform-d) δ=12.33-11.98 (m, 1H), 4.22 (m, 4H), 4.08-3.89 (m, 1H), 3.78 (m, 1H), 3.69-3.39 (m, 4H), 3.25-2.94 (m, 2H), 2.81-2.59 (m, 4H), 2.39-2.25 (m, 2H), 1.95 (br s, 3H), 1.55-1.43 (m, 8H), 1.37 (d, J=6.8 Hz, 3H), 1.31-1.19 (m, 40H), 0.88 (t, J=6.8 Hz, 12H)

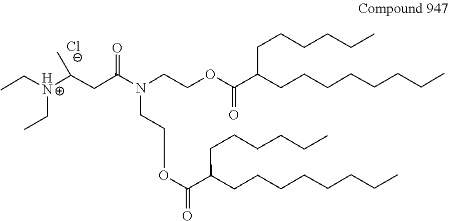

LCMS [M+1]⁺=723.6. ¹H NMR (400 MHz, chloroform-d) δ=11.90-11.72 (m, 1H), 4.33-4.14 (m, 4H), 4.13-4.00 (m, 1H), 3.89-3.77 (m, 1H), 3.61 (br s, 4H), 3.35-3.15 (m, 2H), 3.13-2.91 (m, 2H), 2.83-2.66 (m, 1H), 2.37-2.29 (m, 2H), 1.57 (br s, 3H), 1.56-1.51 (m, 3H), 1.50 (br s, 4H), 1.46 (m, 3H), 1.44 (br s, 4H), 1.34-1.17 (m, 40H), 0.88 (t, J=6.8 Hz, 12H)

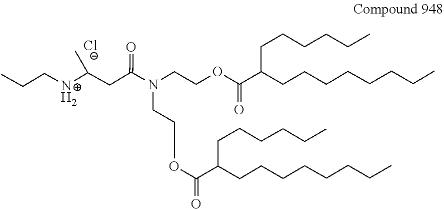 [SMILES: CCCCCCCCC(CCCCCC)C(=O)OCCN(CCOC(=O)C(CCCCCC)CCCCCCCC)C(=O)CC(C)[NH2+]CCC ; [Cl-]]

LCMS [M+1]⁺=709.7. ¹H NMR (400 MHz, chloroform-d) δ=10.91-10.68 (m, 1H), 8.41 (m, 1H), 4.34-4.11 (m, 4H), 3.74-3.67 (m, 1H), 3.67-3.56 (m, 2H), 3.55-3.45 (m, 2H), 3.31-3.09 (m, 2H), 2.95-2.72 (m, 2H), 2.39-2.29 (m, 2H), 1.93-1.81 (m, 2H), 1.64-1.51 (m, 7H), 1.49-1.40 (m, 4H), 1.32-1.20 (m, 40H), 1.07 (t, J=7.2 Hz, 3H), 0.88 (t, J=6.8 Hz, 12H)

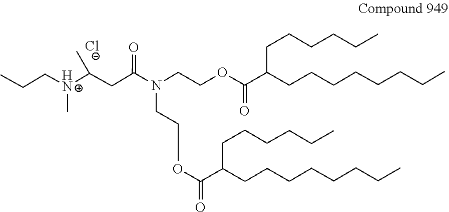

LCMS [M+1]⁺=723.6 ¹H NMR (400 MHz, DMSO-d6) δ=10.23-9.60 (m, 1H), 4.18 (br t, J=5.2 Hz, 2H), 4.13-4.05 (m, 2H), 3.68-3.44 (m, 5H), 3.14-2.86 (m, 3H), 2.80-2.59 (m, 4H), 2.37-2.22 (m, 2H), 1.75-1.64 (m, 2H), 1.54-1.45 (m, 4H), 1.44-1.37 (m, 4H), 1.27 (br s, 3H), 1.25-1.13 (m, 40H), 0.91 (q, J=7.2 Hz, 3H), 0.85 (t, J=6.4 Hz, 12H)

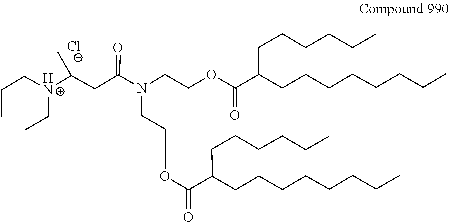

LCMS [M+1]⁺=737.6. ¹H NMR (400 MHz, chloroform-d) δ=11.94-11.59 (m, 1H), 4.33-4.12 (m, 4H), 4.11-3.96 (m, 1H), 3.89-3.75 (m, 1H), 3.73-3.49 (m, 4H), 3.36-3.19 (m, 1H), 3.18-2.96 (m, 2H), 2.95-2.70 (m, 2H), 2.39-2.27 (m, 2H), 1.56 (m, 4H), 1.50 (m, 4H), 1.47-1.39 (m, 6H), 1.35-1.18 (m, 42H), 1.09-0.99 (m, 3H), 0.88 (t, J=6.8 Hz, 12H)

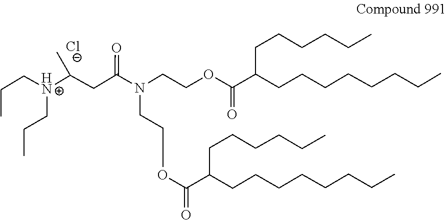

LCMS [M+1]⁺=751.7. ¹H NMR (400 MHz, chloroform-d) δ=11.90-11.68 (m, 1H), 4.40-4.12 (m, 4H), 4.03 (br s, 1H), 3.79 (br s, 1H), 3.53 (br s, 1H), 3.74-3.43 (m, 3H), 3.19-3.00 (m, 2H), 2.97-2.88 (m, 1H), 2.84-2.62 (m, 2H), 2.37-2.28 (m, 2H), 2.20-1.91 (m, 4H), 1.62-1.53 (m, 4H), 1.49-1.40 (m, 7H), 1.31-1.23 (m, 40H), 1.02 (q, J=6.8 Hz, 6H), 0.88 (t, J=6.8 Hz, 12H)

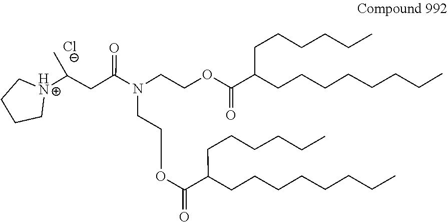

LCMS [M+1]⁺=721.6. ¹H NMR (400 MHz, chloroform-d) δ=12.58-12.22 (m, 1H), 4.20 (br t, J=5.4 Hz, 4H), 3.88-3.72 (m, 3H), 3.70-3.52 (m, 4H), 3.37-3.23 (m, 1H), 2.98-2.80 (m, 3H), 2.40-2.29 (m, 2H), 2.24 (br s, 2H), 2.14-1.98 (m, 2H), 1.62-1.51 (m, 7H), 1.49-1.41 (m, 4H), 1.33-1.21 (m, 40H), 0.88 (t, J=6.8 Hz, 12H)

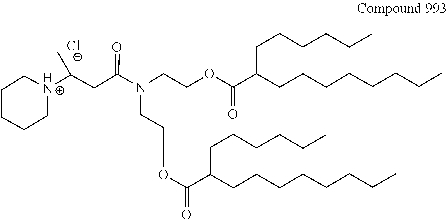

LCMS [M+1]⁺=735.7. ¹H NMR (400 MHz, chloroform-d) δ=11.90 (br s, 1H), 4.33-4.13 (m, 4H), 3.89-3.76 (m, 2H), 3.75-3.51 (m, 4H), 3.51-3.34 (m, 2H), 2.90-2.78 (m, 1H), 2.75-2.62 (m, 2H), 2.51-2.28 (m, 4H), 1.91 (br s, 4H), 1.56 (br d, J=4.1 Hz, 4H), 1.49-1.41 (m, 7H), 1.32-1.22 (m, 40H), 0.88 (t, J=6.8 Hz, 12H)

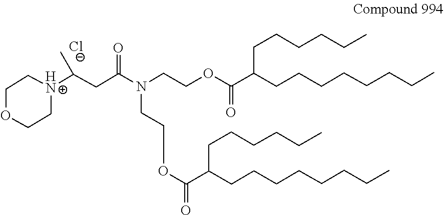

LCMS [M+1]⁺=737.6. ¹H NMR (400 MHz, chloroform-d) δ=12.91 (br s, 1H), 4.57-4.33 (m, 2H), 4.33-4.08 (m, 4H), 3.99 (br t, J=10.8 Hz, 2H), 3.89-3.75 (m, 2H), 3.70-3.49 (m, 4H), 3.45-3.26 (m, 2H), 3.15-2.88 (m, 2H), 2.88-2.69 (m, 1H), 2.39-2.25 (m, 2H), 1.61-1.55 (m, 3H), 1.54-1.39 (m, 8H), 1.33-1.19 (m, 40H), 0.88 (t, J=6.8 Hz, 12H)

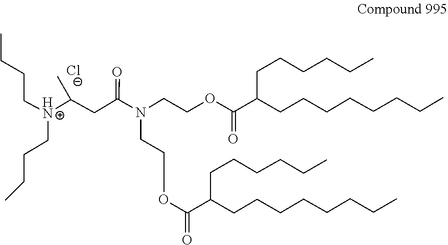

LCMS [M+1]⁺=779.7. ¹H NMR (400 MHz, chloroform-d) δ=11.67 (br s, 1H), 4.21 (br dd, J=5.2, 11.6 Hz, 4H), 4.02 (br d, J=2.4 Hz, 1H), 3.80 (br s, 1H), 3.60 (br s, 4H), 3.09 (br d, J=10.8 Hz, 2H), 2.95 (br d, J=4.4 Hz, 1H), 2.90-2.70 (m, 2H), 2.29-2.13 (m, 2H), 2.04 (br dd, J=2.5, 7.4 Hz, 1H), 1.90 (br s, 2H), 1.81-1.70 (m, 1H), 1.56 (br d, J=4.4 Hz, 4H), 1.42 (br d, J=4.0 Hz, 11H), 1.26 (br s, 40H), 0.99 (br s, 6H), 0.93-0.80 (m, 12H)

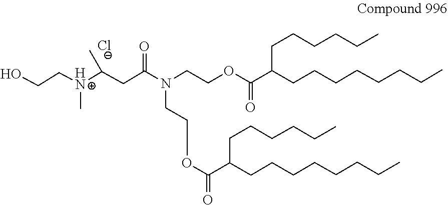

LCMS [M+1]⁺=725.6. ¹H NMR (400 MHz, DMSO-d₆) δ=9.74 (m, 1H), 4.18 (br d, J=3.6 Hz, 2H), 4.13-4.04 (m, 2H), 3.86-3.79 (m, 1H), 3.78-3.73 (m, 2H), 3.69-3.62 (m, 2H), 3.60-3.47 (m, 3H), 3.33-3.08 (m, 2H), 3.04-2.92 (m, 1H), 2.81-2.75 (m, 2H), 2.74-2.66 (m, 2H), 2.35-2.24 (m, 2H), 1.53-1.44 (m, 4H), 1.43-1.35 (m, 4H), 1.29-1.16 (m, 43H), 0.85 (t, J=6.4 Hz, 12H)

### Series 900-6 Compounds

Synthesis and Characterization of Compounds 960a-g, 961-969:

Common intermediates: Compounds A-C were synthesized in the same batch as the precursors used for the preparation of Compounds 901, 902, and 903, respectively. Compounds D-F were all prepared under the same reaction conditions.

### Synthesis and Characterization of Common Intermediates, Compounds D-F:

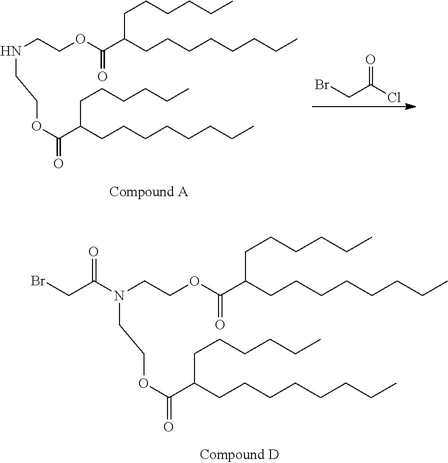 [SMILES: CCCCCCCCC(CCCCCC)C(=O)OCCNCCOC(=O)C(CCCCCC)CCCCCCCC ; O=C(Cl)CBr ; CCCCCCCCC(CCCCCC)C(=O)OCCN(CCOC(=O)C(CCCCCC)CCCCCCCC)C(=O)CBr]

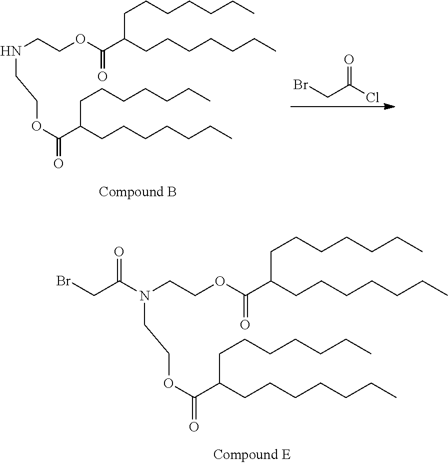 [SMILES: CCCCCCCC(CCCCCCC)C(=O)OCCNCCOC(=O)C(CCCCCCC)CCCCCCC ; O=C(Cl)CBr ; CCCCCCCC(CCCCCCC)C(=O)OCCN(CCOC(=O)C(CCCCCCC)CCCCCCC)C(=O)CBr]

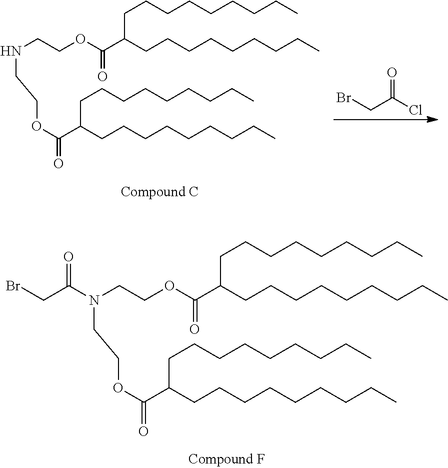 [SMILES: CCCCCCCCCC(CCCCCCCCC)C(=O)OCCNCCOC(=O)C(CCCCCCCCC)CCCCCCCCC ; O=C(Cl)CBr ; CCCCCCCCCC(CCCCCCCCC)C(=O)OCCN(CCOC(=O)C(CCCCCCCCC)CCCCCCCCC)C(=O)CBr]

### Representative Synthetic Preparation of Compound D

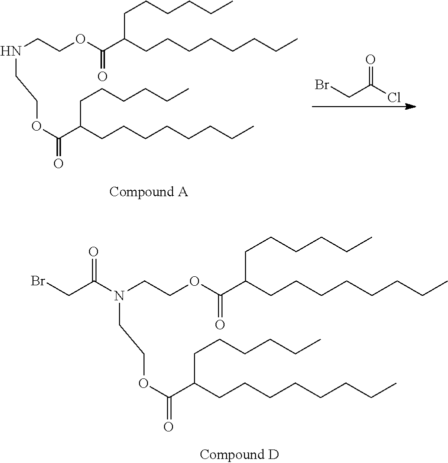 [SMILES: CCCCCCCCC(CCCCCC)C(=O)OCCNCCOC(=O)C(CCCCCC)CCCCCCCC ; O=C(Cl)CBr ; CCCCCCCCC(CCCCCC)C(=O)OCCN(CCOC(=O)C(CCCCCC)CCCCCCCC)C(=O)CBr]

To a solution of Compound A (4 g, 6.87 mmol, 1 eq.) in DCM (40 mL) was added bromoacetyl chloride (1.62 g, 10.31 mmol, 858.60 μL, 1.5 eq.) at 0° C. The mixture was stirred at 20° C. for 1 hour. TLC (Petroleum ether:Ethyl acetate=5:1, R_{f}=0.6) indicated Compound A was consumed completely and new spots formed. The reaction mixture was concentrated under reduced pressure to give a residue. The residue was purified by column chromatography (SiO₂, Petroleum ether:Ethyl acetate=98:2 to 94:6) to give Compound D (5.13 g, 7.30 mmol, 53.09% yield, 100% purity) as white solid.

¹H NMR (400 MHz, CHLOROFORM-d) δ=4.28-4.21 (m, 4H), 4.17 (s, 1H), 3.95 (s, 1H), 3.74-3.61 (m, 4H), 2.37-2.29 (m, 2H), 1.57 (s, 4H), 1.45 (m, 4H), 1.37-1.14 (m, 40H), 0.89 (t, J=6.8 Hz, 12H)

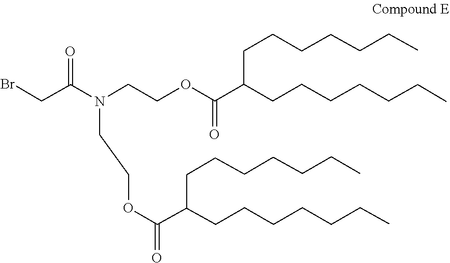 [SMILES: CCCCCCCC(CCCCCCC)C(=O)OCCN(CCOC(=O)C(CCCCCCC)CCCCCCC)C(=O)CBr]

Compound E. ¹H NMR (400 MHz, CHLOROFORM-d) δ=4.29-4.21 (m, 4H), 4.17 (s, 1H), 3.95 (s, 1H), 3.76-3.54 (m, 4H), 2.33 (ddd, J=3.2, 5.6, 8.8 Hz, 2H), 1.57-1.52 (m, 4H), 1.50-1.40 (m, 4H), 1.33-1.21 (m, 40H), 0.89 (t, J=6.8 Hz, 12H)

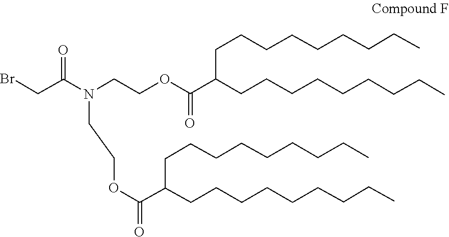 [SMILES: CCCCCCCCCC(CCCCCCCCC)C(=O)OCCN(CCOC(=O)C(CCCCCCCCC)CCCCCCCCC)C(=O)CBr]

### Compound F. ¹H NMR (400 MHz, CHLOROFORM-d) δ=4.29-4.19 (m, 4H), 4.17 (s, 1H), 3.95 (s, 1H), 3.77-3.58 (m, 4H), 2.40-2.27 (m, 2H), 1.58-1.51 (m, 4H), 1.45 (m, 4H), 1.38-1.08 (m, 56H), 0.89 (t, J=6.8 Hz, 12H).

Synthesis of compounds 960a-g and 961-969: All compounds were synthesized with the same procedure using 5 equiv. of amine, except for Compound 961 which used 8 eq amine.

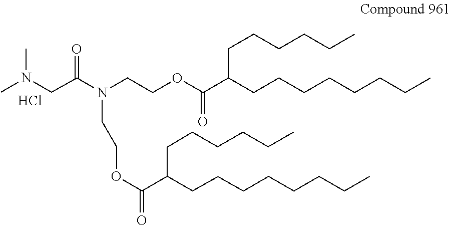 [SMILES: CCCCCCCCC(CCCCCC)C(=O)OCCN(CCOC(=O)C(CCCCCC)CCCCCCCC)C(=O)CN(C)C ; Cl]

Prepared in the same way from common intermediates. Compounds 977a-e were synthesized using 30 equiv. of amine, while Compounds 977f-p were prepared using 20 equiv. of amine.

### Representative Synthesis of Compound 961

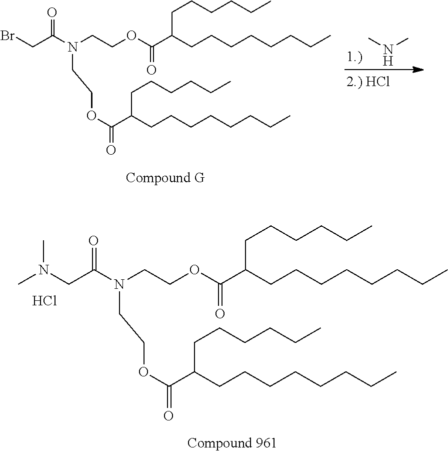

To a solution of Compound D (200 mg, 284.54 μmol, 1 equiv.) in DCM (0.5 mL) was added dimethylamine (2 M, 1.14 mL, 8 eq.). The mixture was stirred at 20° C. for 16 hours. TLC (Dichloromethane:Methanol=10:1, R_{f}=0.71) indicated Compound D was consumed and new spots formed. The reaction mixture was concentrated under reduced pressure to give a residue. The residue was purified by column chromatography (SiO₂, Dichloromethane:Methanol=300:1 to 150:1) to give a colorless oil (160 mg, 239.86 μmol, 84.30% yield, 100% purity).

LCMS [M+1]⁺=667.7. ¹H NMR (400 MHz, CHLOROFORM-d) δ=4.22 (q, J=5.6 Hz, 4H), 3.79 (t, J=6.0 Hz, 2H), 3.62 (t, J=5.6 Hz, 2H), 3.21 (s, 2H), 2.37-2.28 (m, 8H), 1.65-1.52 (m, 4H), 1.50-1.39 (m, 4H), 1.34-1.18 (m, 40H), 0.88 (t, J=6.8 Hz, 12H) A portion of this oil (70 mg, 104.94 μmol, 1 eq.) was dissolved in HCl/H₂O (20 mL, 0.04 wt %) and stirred at 20° C. for 1 hour. The mixture was lyophilized to give Compound 961 (31.10 mg, 44.21 μmol, 42.13% yield, 100% purity, HCl) as a white gum.

LCMS [M+1]⁺=667.6. ¹H NMR (400 MHz, DMSO-d₆) δ=9.66 (br s, 1H), 4.26 (br s, 2H), 4.20 (br t, J=5.2 Hz, 2H), 4.13 (br t, J=5.6 Hz, 2H), 3.59 (br dd, J=5.2, 10.4 Hz, 4H), 2.80 (s, 6H), 2.41-2.21 (m, 2H), 1.48 (m, 4H), 1.45-1.36 (m, 4H), 1.22 (br s, 40H), 0.85 (br t, J=6.8 Hz, 12H)

Characterization of Compounds 960b-960g, 962-969

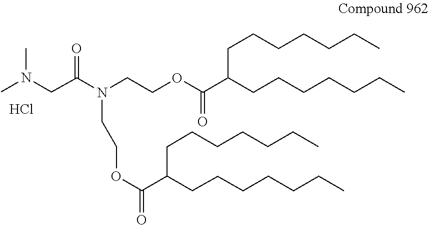 [SMILES: CCCCCCCC(CCCCCCC)C(=O)OCCN(CCOC(=O)C(CCCCCCC)CCCCCCC)C(=O)CN(C)C ; Cl]

### Compound 962. ¹H NMR (400 MHz, CHLOROFORM-d) δ=12.95 (s, 1H), 4.15 (td, J=5.2, 19.2 Hz, 4H), 3.96 (s, 2H), 3.63-3.41 (m, 4H), 2.98 (d, J=3.6 Hz, 6H), 2.34-2.20 (m, 2H), 1.52-1.34 (m, 8H), 1.25-1.12 (m, 40H), 0.90-0.74 (m, 12H)

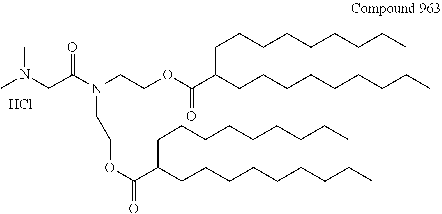 [SMILES: CCCCCCCCCC(CCCCCCCCC)C(=O)OCCN(CCOC(=O)C(CCCCCCCCC)CCCCCCCCC)C(=O)CN(C)C ; Cl]

### Compound 963. ¹H NMR (400 MHz, CHLOROFORM-d) δ=12.98 (m, 1H), 4.23 (br d, J=18.0 Hz, 4H), 4.05 (br s, 2H), 3.69-3.47 (m, 4H), 3.06 (br s, 6H), 2.39-2.23 (m, 2H), 1.62-1.52 (m, 4H), 1.51-1.40 (m, 4H), 1.36-1.16 (m, 56H), 0.88 (t, J=6.8 Hz, 12H).

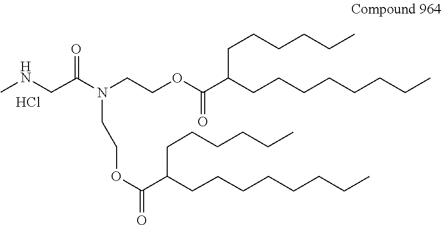 [SMILES: CCCCCCCCC(CCCCCC)C(=O)OCCN(CCOC(=O)C(CCCCCC)CCCCCCCC)C(=O)CNC ; Cl]

Compound 964. ¹H NMR (400 MHz, CHLOROFORM-d) δ=9.66 (br s, 2H), 4.24 (br d, J=4.4 Hz, 4H), 4.06 (br s, 2H), 3.71-3.51 (m, 4H), 2.87 (br s, 3H), 2.41-2.28 (m, 2H), 1.58 (br dd, J=7.2, 13.6 Hz, 4H), 1.51-1.40 (m, 4H), 1.35-1.19 (m, 40H), 0.89 (t, J=6.8 Hz, 12H)

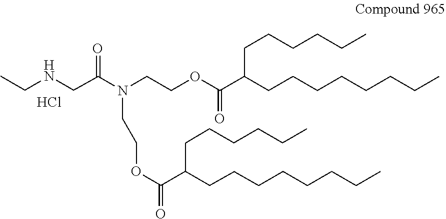 [SMILES: CCCCCCCCC(CCCCCC)C(=O)OCCN(CCOC(=O)C(CCCCCC)CCCCCCCC)C(=O)CNCC ; Cl]

### Compound 965. ¹H NMR (400 MHz, CHLOROFORM-d) δ=9.55 (br s, 2H), 4.24 (br s, 4H), 4.06 (br s, 2H), 3.75-3.51 (m, 4H), 3.28-3.11 (m, 2H), 2.33 (br d, J=5.2 Hz, 2H), 1.63-1.50 (m, 7H), 1.47 (m, 4H), 1.36-1.16 (m, 40H), 0.89 (t, J=6.7 Hz, 12H)

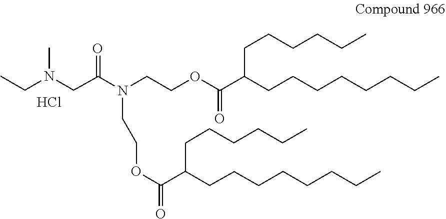 [SMILES: CCCCCCCCC(CCCCCC)C(=O)OCCN(CCOC(=O)C(CCCCCC)CCCCCCCC)C(=O)CN(C)CC ; Cl]

Compound 966. ¹H NMR (400 MHz, CHLOROFORM-d) δ=12.86-12.70 (m, 1H), 4.30-4.15 (m, 4H), 4.12-3.93 (m, 2H), 3.67-3.54 (m, 4H), 3.54-3.45 (m, 2H), 3.02 (d, J=4.4 Hz, 3H), 2.37-2.28 (m, 2H), 1.56 (m, 4H), 1.51-1.41 (m, 7H), 1.35-1.16 (m, 40H), 0.89 (t, J=6.8 Hz, 12H)

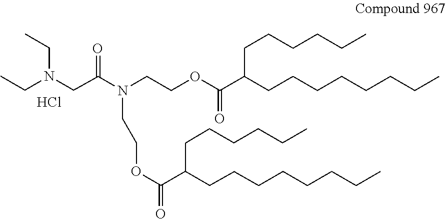 [SMILES: CCCCCCCCC(CCCCCC)C(=O)OCCN(CCOC(=O)C(CCCCCC)CCCCCCCC)C(=O)CN(CC)CC ; Cl]

Compound 967. ¹H NMR (400 MHz, CHLOROFORM-d) δ=12.49-12.36 (m, 1H), 4.29-4.16 (m, 4H), 4.12-4.00 (m, 2H), 3.68-3.57 (m, 4H), 3.57-3.41 (m, 4H), 2.38-2.27 (m, 2H), 1.57 (br s, 4H), 1.52-1.40 (m, 10H), 1.35-1.16 (m, 40H), 0.89 (t, J=6.8 Hz, 12H)

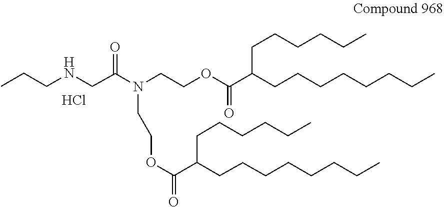 [SMILES: CCCCCCCCC(CCCCCC)C(=O)OCCN(CCOC(=O)C(CCCCCC)CCCCCCCC)C(=O)CNCCC ; Cl]

Compound 968. ¹H NMR (400 MHz, CHLOROFORM-d) δ=9.54 (br s, 2H), 4.24 (br s, 4H), 4.06 (br s, 2H), 3.73-3.53 (m, 4H), 3.05 (br s, 2H), 2.40-2.27 (m, 2H), 1.96 (br d, J=6.4 Hz, 2H), 1.56 (br d, J=6.4 Hz, 4H), 1.46 (br d, J=6.8 Hz, 4H), 1.37-1.15 (m, 40H), 1.06 (br t, J=7.2 Hz, 3H), 0.88 (t, J=6.8 Hz, 12H).

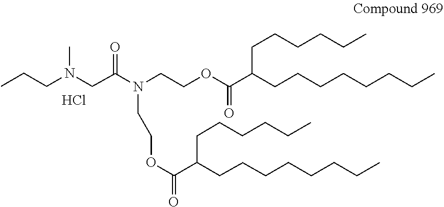 [SMILES: CCCCCCCCC(CCCCCC)C(=O)OCCN(CCOC(=O)C(CCCCCC)CCCCCCCC)C(=O)CN(C)CCC ; Cl]

Compound 969. ¹H NMR (400 MHz, CHLOROFORM-d) δ=12.73 (s, 1H), 4.30-4.15 (m, 4H), 4.03 (d, J=11.2 Hz, 2H), 3.68-3.52 (m, 4H), 3.40-3.28 (m, 2H), 3.02 (d, J=4.0 Hz, 3H), 2.37-2.27 (m, 2H), 1.89 (td, J=7.6, 15.2 Hz, 2H), 1.59-1.41 (m, 8H), 1.36-1.12 (m, 40H), 1.02 (t, J=7.2 Hz, 3H), 0.89 (t, J=6.8 Hz, 12H)

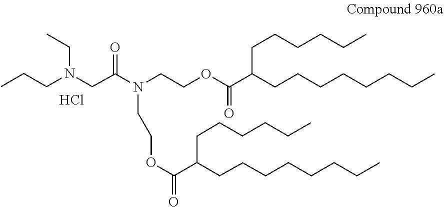 [SMILES: CCCCCCCCC(CCCCCC)C(=O)OCCN(CCOC(=O)C(CCCCCC)CCCCCCCC)C(=O)CN(CC)CCC ; Cl]

Compound 960a. ¹H NMR (400 MHz, DMSO-d₆) δ=9.31 (br s, 1H), 4.25 (br s, 1H), 4.20 (br t, J=5.6 Hz, 2H), 4.14 (br t, J=6.0 Hz, 2H), 3.69-3.53 (m, 4H), 3.25-2.95 (m, 4H), 2.35-2.23 (m, 2H), 1.72-1.56 (m, 2H), 1.53-1.35 (m, 8H), 1.22 (br s, 43H), 0.95-0.77 (m, 15H)

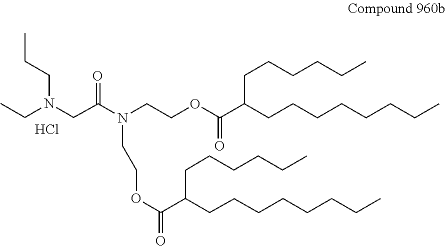 [SMILES: CCCCCCCCC(CCCCCC)C(=O)OCCN(CCOC(=O)C(CCCCCC)CCCCCCCC)C(=O)CN(CC)CCC ; Cl]

Compound 960b. ¹H NMR (400 MHz, DMSO-d₆) δ=9.36-9.18 (m, 1H), 4.27 (s, 2H), 4.23-4.10 (m, 4H), 3.61 (s, 4H), 3.04 (s, 4H), 2.36-2.24 (m, 2H), 1.75-1.58 (m, 4H), 1.55-1.45 (m, 4H), 1.45-1.37 (m, 4H), 1.23 (s, 40H), 0.95-0.78 (m, 18H)

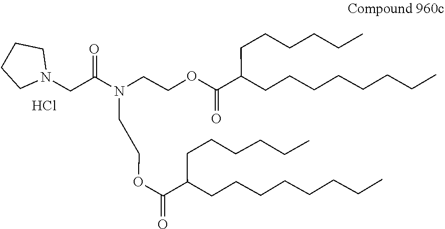 [SMILES: CCCCCCCCC(CCCCCC)C(=O)OCCN(CCOC(=O)C(CCCCCC)CCCCCCCC)C(=O)CN1CCCC1 ; Cl]

Compound 960c. ¹H NMR (400 MHz, CHLOROFORM-d) δ=12.75 (br s, 1H), 4.24 (s, 4H), 4.13 (br s, 2H), 3.72 (br s, 2H), 3.67-3.51 (m, 4H), 3.43-3.29 (m, 2H), 2.39-2.27 (m, 2H), 2.20 (br s, 4H), 1.62-1.51 (m, 4H), 1.50-1.39 (m, 4H), 1.36-1.14 (m, 40H), 0.88 (t, J=6.8 Hz, 12H)

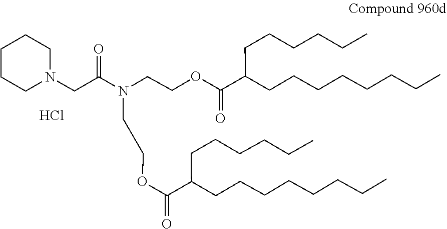 [SMILES: CCCCCCCCC(CCCCCC)C(=O)OCCN(CCOC(=O)C(CCCCCC)CCCCCCCC)C(=O)CN1CCCCC1 ; Cl]

Compound 960d. ¹H NMR (400 MHz, CHLOROFORM-d) δ=12.27 (m, 1H), 4.29-4.14 (m, 4H), 3.99 (br s, 2H), 3.62 (br t, J=5.2 Hz, 6H), 3.36 (br d, J=10.0 Hz, 2H), 2.37-2.22 (m, 4H), 1.95-1.81 (m, 3H), 1.62-1.52 (m, 4H), 1.51-1.39 (m, 5H), 1.35-1.16 (m, 40H), 0.88 (t, J=6.8 Hz, 12H)

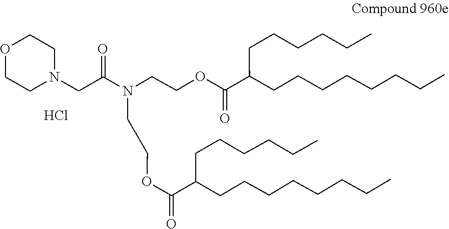 [SMILES: CCCCCCCCC(CCCCCC)C(=O)OCCN(CCOC(=O)C(CCCCCC)CCCCCCCC)C(=O)CN1CCOCC1 ; Cl]

Compound 960e. ¹H NMR (400 MHz, CHLOROFORM-d) δ=4.23 (q, J=5.6 Hz, 4H), 3.82-3.69 (m, 6H), 3.61 (t, J=5.6 Hz, 2H), 3.27 (br s, 2H), 2.56 (br s, 4H), 2.32 (br dd, J=3.2, 8.8 Hz, 2H), 1.64-1.55 (m, 4H), 1.50-1.41 (m, 4H), 1.34-1.21 (m, 40H), 0.88 (t, J=6.8 Hz, 12H)

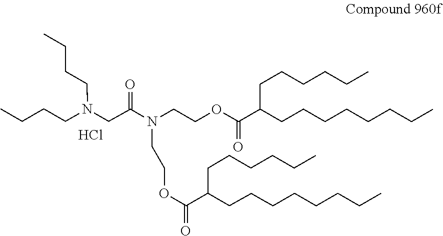 [SMILES: CCCCCCCCC(CCCCCC)C(=O)OCCN(CCOC(=O)C(CCCCCC)CCCCCCCC)C(=O)CN(CCCC)CCCC ; Cl]

### Compound 960f. ¹H NMR (400 MHz, CHLOROFORM-d) δ=13.75-12.88 (m, 1H), 4.37-4.15 (m, 6H), 3.99 (br s, 4H), 3.85-3.53 (m, 6H), 3.50-3.24 (m, 2H), 2.37-2.27 (m, 2H), 1.59-1.51 (m, 4H), 1.47 (m, 4H), 1.35-1.15 (m, 40H), 0.89 (t, J=6.8 Hz, 12H)

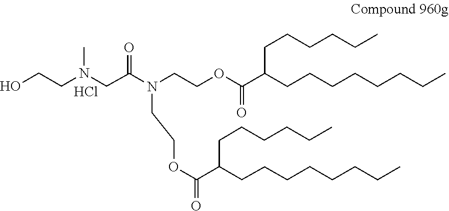 [SMILES: CCCCCCCCC(CCCCCC)C(=O)OCCN(CCOC(=O)C(CCCCCC)CCCCCCCC)C(=O)CN(C)CCO ; Cl]

Compound 960g. ¹H NMR (400 MHz, CHLOROFORM-d) δ=12.36-11.65 (m, 1H), 4.39 (br d, J=16.8 Hz, 1H), 4.32-4.18 (m, 4H), 4.18-4.04 (m, 2H), 3.94 (br dd, J=5.6, 13.6 Hz, 1H), 3.76-3.42 (m, 6H), 3.13 (s, 3H), 2.40-2.28 (m, 2H), 1.63-1.52 (m, 4H), 1.46 (m, 4H), 1.36-1.16 (m, 40H), 0.89 (t, J=6.8 Hz, 12H)

### Representative Synthesis and Characterization of Compounds 977a-p:

Common intermediates: Synthesis of Compounds A-C were described above. Compounds G-I were prepared under the same reaction conditions.

### Representative Synthesis and Characterization of Common Intermediates, Compounds G-I:

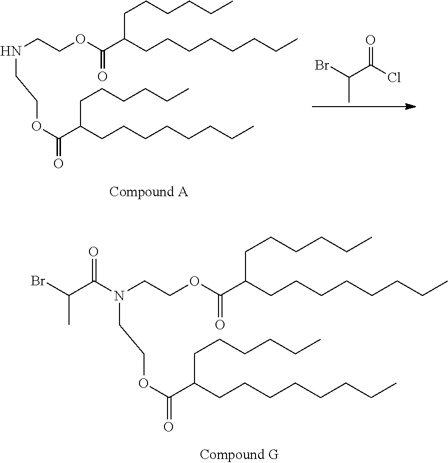

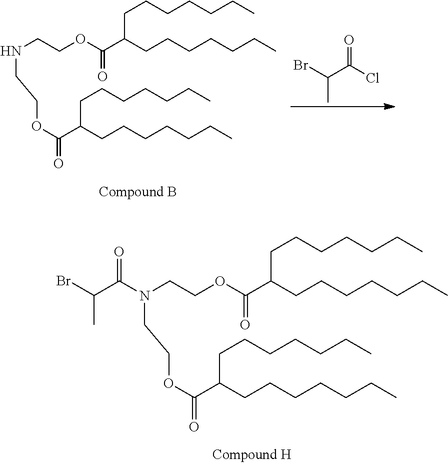

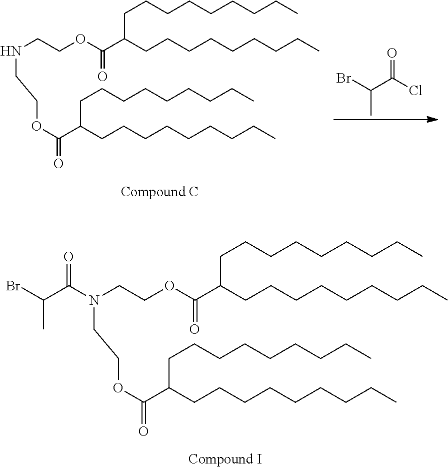

### Representative Preparation of Compounds G-I: Preparation of Compound H

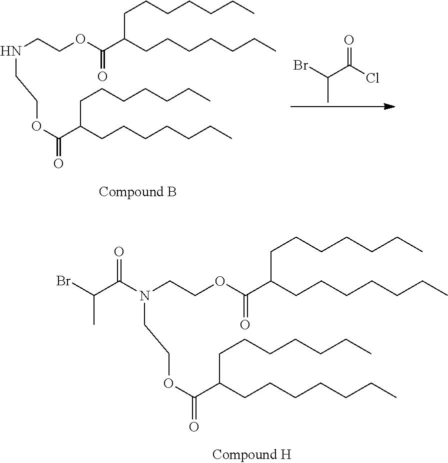

To a solution of Compound B (950 mg, 1.63 mmol, 1 eq.) in DCM (8.5 mL) was added TEA (825.93 mg, 8.16 mmol, 1.14 mL, 5 eq.) and 2-bromopropionyl chloride (559.67 mg, 3.26 mmol, 329.22 μL, 2 eq.) at −20° C., and the mixture was stirred at 25 for 0.5 hour. LCMS showed Compound B was consumed completely and one main peak with desired MS was detected. The reaction mixture was concentrated under reduced pressure to give a residue. The residue was purified by column chromatography (SiO₂, Petroleum ether:Ethyl acetate=50:1 to 10:1) to give Compound H (670 mg, 934.57 μmol, 57.25% yield) as a colorless oil.

¹H NMR (400 MHz, CHLOROFORM-d) δ=4.67 (d, J=6.8 Hz, 1H), 4.30-4.14 (m, 4H), 4.06-3.79 (m, 2H), 3.58-3.32 (m, 2H), 2.38-2.24 (m, 2H), 1.83 (d, J=6.8 Hz, 3H), 1.68-1.52 (m, 4H), 1.49-1.39 (m, 4H), 1.35-1.18 (m, 40H), 0.88 (t, J=6.8 Hz, 12H)

### Compound G

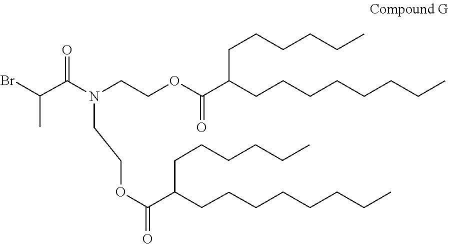 [SMILES: CCCCCCCCC(CCCCCC)C(=O)OCCN(CCOC(=O)C(CCCCCC)CCCCCCCC)C(=O)C(C)Br]

¹H NMR (400 MHz, CHLOROFORM-d) δ=4.65 (s, 1H), 4.31-4.15 (m, 4H), 4.04-3.92 (m, 1H), 3.87 (td, J=5.2, 14.4 Hz, 1H), 3.56-3.45 (m, 1H), 3.44-3.35 (m, 1H), 2.37-2.26 (m, 2H), 1.83 (d, J=6.4 Hz, 3H), 1.60-1.52 (m, 4H), 1.49-1.40 (m, 4H), 1.31-1.19 (m, 40H), 0.87-0.87 (m, 1H), 0.88 (t, J=6.8 Hz, 12H)

### Compound I

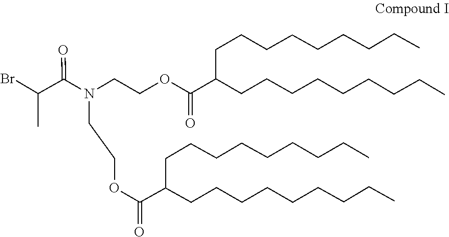 [SMILES: CCCCCCCCCC(CCCCCCCCC)C(=O)OCCN(CCOC(=O)C(CCCCCCCCC)CCCCCCCCC)C(=O)C(C)Br]

¹H NMR (400 MHz, CHLOROFORM-d) δ=4.67 (d, J=6.4 Hz, 1H), 4.33-4.14 (m, 4H), 4.04-3.83 (m, 2H), 3.58-3.35 (m, 2H), 2.39-2.26 (m, 2H), 1.84 (d, J=6.5 Hz, 3H), 1.59 (br s, 2H), 1.56-1.52 (m, 2H), 1.50-1.41 (m, 4H), 1.33-1.22 (m, 56H), 0.89 (t, J=6.8 Hz, 12H)

Syntheses of Compounds 977a-977p: All compounds prepared in the same way from common intermediates. Compounds 977a-e were synthesized using 30 equiv of amine, while Compounds 977f-p were prepared using 20 equiv of amine.

### Representative Synthesis and Characterization of Compound 977a

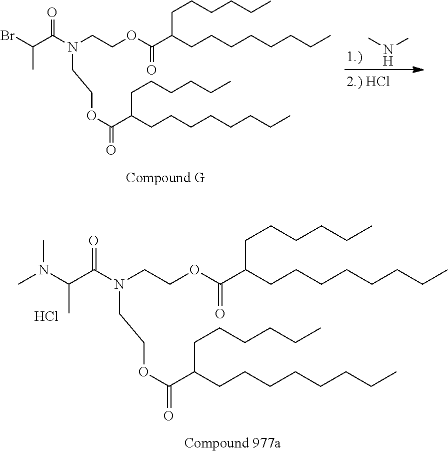

1.) To a solution of Compound G (350 mg, 488.21 μmol, 1 eq.) in DMF (2 mL) was added K₂CO₃ (337.38 mg, 2.44 mmol, 5 eq.), KI (162.08 mg, 976.41 μmol, 2 eq.) and dimethylamine (2 M, 7.32 mL, 30 eq.). The mixture was stirred at 70° C. for 12 hours. TLC (DCM: MeOH=10:1, R_{f}=0.45) indicated Compound 6 was consumed completely and new spots formed. The mixture was filtered and the filtrate was concentrated under reduced pressure to give a residue. The residue was purified by column chromatography (SiO₂, DCM:MeOH=100:1 to 30:1) to give a yellow oil (220 mg, 323.02 μmol, 66.16% yield). LCMS [M+1]⁺=681.7

2.) To a portion of this oil (200 mg, 293.65 μmol, 1 eq.) in DCM (0.25 mL) was added HCl/dioxane (0.25 mL) at 25° C. The reaction mixture was stirred at 25° C. for 0.5 hour. The reaction mixture was lyophilized to give Compound 977a (200 mg, 278.73 μmol, 94.92% yield, HCl) as a yellow oil.

¹H NMR (400 MHz, CHLOROFORM-d) δ=12.75 (br s, 1H), 4.44 (br d, J=4.8 Hz, 1H), 4.31-4.15 (m, 4H), 3.83-3.47 (m, 4H), 3.05 (br d, J=4.0 Hz, 3H), 2.85 (br d, J=3.6 Hz, 3H), 2.38-2.24 (m, 2H), 1.68 (br d, J=5.6 Hz, 3H), 1.56 (br d, J=5.6 Hz, 4H), 1.50-1.40 (m, 4H), 1.31-1.19 (m, 40H), 0.88 (t, J=6.8 Hz, 12H)

Characterization of Compounds 977b-977p:

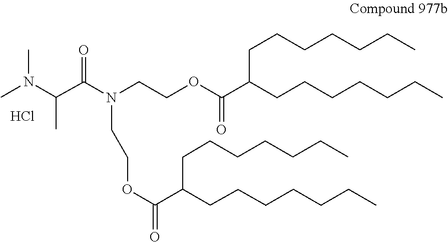 [SMILES: CCCCCCCC(CCCCCCC)C(=O)OCCN(CCOC(=O)C(CCCCCCC)CCCCCCC)C(=O)C(C)N(C)C ; Cl]

Compound 977b. ¹H NMR (400 MHz, CHLOROFORM-d) δ=12.87-12.66 (m, 1H), 4.44 (d, J=5.2 Hz, 1H), 4.31-4.17 (m, 4H), 3.84-3.67 (m, 2H), 3.66-3.48 (m, 2H), 3.04 (d, J=4.4 Hz, 3H), 2.85 (d, J=4.4 Hz, 3H), 2.31 (dd, J=4.4, 8.8 Hz, 2H), 1.68 (d, J=6.8 Hz, 3H), 1.62-1.51 (m, 4H), 1.50-1.41 (m, 4H), 1.33-1.19 (m, 40H), 0.94-0.83 (m, 12H)

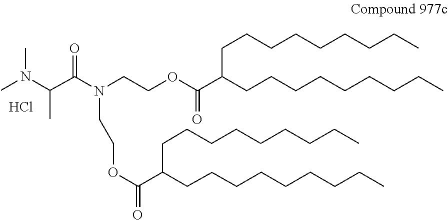 [SMILES: CCCCCCCCCC(CCCCCCCCC)C(=O)OCCN(CCOC(=O)C(CCCCCCCCC)CCCCCCCCC)C(=O)C(C)N(C)C ; Cl]

Compound 977c. ¹H NMR (400 MHz, CHLOROFORM-d) δ=12.83 (br s, 1H), 4.41 (br s, 1H), 4.24 (d, J=5.4 Hz, 4H), 3.82-3.50 (m, 4H), 3.05 (d, J=4.4 Hz, 3H), 2.85 (d, J=4.4 Hz, 3H), 2.31 (br s, 2H), 1.68 (d, J=6.4 Hz, 3H), 1.61-1.52 (m, 4H), 1.46 (m, 4H), 1.33-1.19 (m, 56H), 0.88 (t, J=6.8 Hz, 12H)

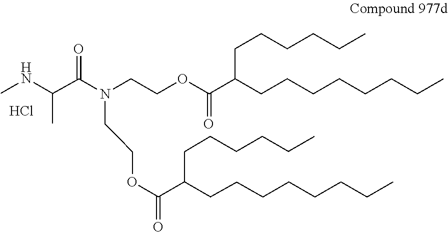 [SMILES: CCCCCCCCC(CCCCCC)C(=O)OCCN(CCOC(=O)C(CCCCCC)CCCCCCCC)C(=O)C(C)NC ; Cl]

Compound 977d. ¹H NMR (400 MHz, CHLOROFORM-d) δ=9.49 (br s, 1H), 8.87 (br s, 1H), 4.33 (br s, 1H), 4.24-4.17 (m, 2H), 4.17-4.06 (m, 2H), 3.78-3.70 (m, 1H), 3.68-3.59 (m, 2H), 3.50-3.44 (m, 1H), 2.47 (t, J=5.2 Hz, 3H), 2.29 (d, J=8.0 Hz, 2H), 1.47 (m, 4H), 1.43-1.32 (m, 7H), 1.22 (br s, 40H), 0.85 (t, J=6.5 Hz, 12H)

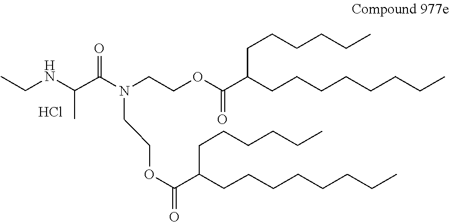 [SMILES: CCCCCCCCC(CCCCCC)C(=O)OCCN(CCOC(=O)C(CCCCCC)CCCCCCCC)C(=O)C(C)NCC ; Cl]

Compound 977e. ¹H NMR (400 MHz, CHLOROFORM-d) δ=11.29-11.07 (m, 1H), 7.76 (m, 1H), 4.40-4.29 (m, 1H), 4.29-4.15 (m, 4H), 3.93-3.80 (m, 1H), 3.73-3.56 (m, 2H), 3.56-3.43 (m, 1H), 3.27-3.11 (m, 1H), 3.04-2.87 (m, 1H), 2.37-2.26 (m, 2H), 1.77-1.71 (m, 3H), 1.61-1.41 (m, 11H), 1.32-1.19 (m, 40H), 0.88 (t, J=6.8 Hz, 12H)

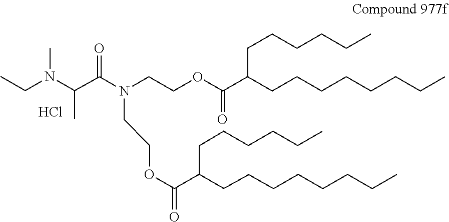 [SMILES: CCCCCCCCC(CCCCCC)C(=O)OCCN(CCOC(=O)C(CCCCCC)CCCCCCCC)C(=O)C(C)N(C)CC ; Cl]

Compound 977f. ¹H NMR (400 MHz, CHLOROFORM-d) δ=12.40 (m, 1H), 4.48 (m, 1H), 4.23 (br s, 4H), 3.84-3.40 (m, 5H), 3.30-3.12 (m, 1H), 3.03 (br s, 2H), 2.83 (br s, 1H), 2.31 (br d, J=4.0 Hz, 2H), 1.68 (br s, 3H), 1.56 (br s, 4H), 1.52-1.42 (m, 7H), 1.25 (br s, 40H), 0.88 (t, J=6.8 Hz, 12H)

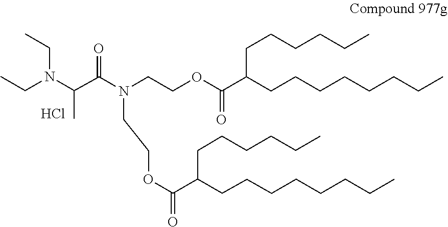 [SMILES: CCCCCCCCC(CCCCCC)C(=O)OCCN(CCOC(=O)C(CCCCCC)CCCCCCCC)C(=O)C(C)N(CC)CC ; Cl]

Compound 977g. ¹H NMR (400 MHz, CHLOROFORM-d) δ=12.05 (br s, 1H), 4.54 (d, J=6.8 Hz, 1H), 4.31-4.18 (m, 4H), 3.71 (d, J=14.4 Hz, 2H), 3.66-3.54 (m, 3H), 3.48 (d, J=4.8 Hz, 1H), 3.29 (d, J=5.6 Hz, 2H), 2.31 (dt, J=2.8, 5.6 Hz, 2H), 1.74 (d, J=6.8 Hz, 3H), 1.54 (t, J=7.2 Hz, 7H), 1.51-1.39 (m, 7H), 1.33-1.18 (m, 40H), 0.88 (t, J=6.8 Hz, 12H)

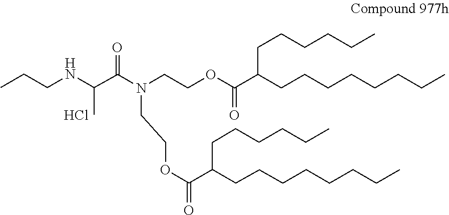 [SMILES: CCCCCCCCC(CCCCCC)C(=O)OCCN(CCOC(=O)C(CCCCCC)CCCCCCCC)C(=O)C(C)NCCC ; Cl]

Compound 977h. ¹H NMR (400 MHz, DMSO-d₆) δ=9.81 (br s, 1H), 8.85 (br d, J=8.4 Hz, 1H), 4.46-4.27 (m, 1H), 4.24-4.08 (m, 4H), 3.77-3.59 (m, 3H), 3.53-3.47 (m, 1H), 2.80 (m, 1H), 2.69-2.59 (m, 1H), 2.34-2.24 (m, 2H), 1.73-1.61 (m, 2H), 1.54-1.44 (m, 4H), 1.44-1.36 (m, 7H), 1.22 (br s, 40H), 0.92-0.81 (m, 15H)

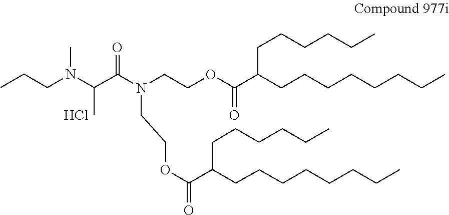 [SMILES: CCCCCCCCC(CCCCCC)C(=O)OCCN(CCOC(=O)C(CCCCCC)CCCCCCCC)C(=O)C(C)N(C)CCC ; Cl]

Compound 977i. ¹H NMR (400 MHz, DMSO-d₆) δ=10.73-9.89 (m, 1H), 4.73-4.45 (m, 1H), 4.34-4.06 (m, 4H), 3.82-3.63 (m, 3H), 3.51 (br dd, J=6.8, 13.6 Hz, 1H), 3.22-2.85 (m, 2H), 2.83-2.71 (m, 3H), 2.35-2.23 (m, 2H), 1.78-1.64 (m, 2H), 1.54-1.35 (m, 11H), 1.22 (br s, 40H), 0.94-0.78 (m, 15H)

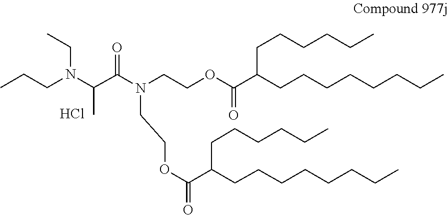 [SMILES: CCCCCCCCC(CCCCCC)C(=O)OCCN(CCOC(=O)C(CCCCCC)CCCCCCCC)C(=O)C(C)N(CC)CCC ; Cl]

Compound 977j. ¹H NMR (400 MHz, CHLOROFORM-d) δ=12.13-11.79 (m, 1H), 4.54 (m, 1H), 4.23 (br t, J=5.6 Hz, 4H), 3.79-3.67 (m, 2H), 3.64-3.54 (m, 2H), 3.48 (m, 1H), 3.35-3.04 (m, 3H), 2.31 (br dd, J=5.6, 8.4 Hz, 2H), 2.06-1.87 (m, 2H), 1.74 (br d, J=5.6 Hz, 6H), 1.55-1.40 (m, 8H), 1.32-1.18 (m, 40H), 1.04-0.96 (m, 3H), 0.88 (t, J=6.8 Hz, 12H)

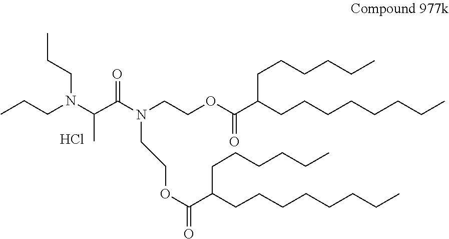 [SMILES: CCCCCCCCC(CCCCCC)C(=O)OCCN(CCOC(=O)C(CCCCCC)CCCCCCCC)C(=O)C(C)N(CCC)CCC ; Cl]

Compound 977k. ¹H NMR (400 MHz, DMSO-d₆) δ=10.89-9.68 (m, 1H), 4.56-4.45 (m, 1H), 4.26-4.05 (m, 4H), 3.82-3.56 (m, 3H), 3.55-3.48 (m, 1H), 3.25-3.11 (m, 1H), 3.07-2.91 (m, 2H), 2.87-2.72 (m, 1H), 2.35-2.23 (m, 2H), 1.70 (m, 4H), 1.55-1.45 (m, 4H), 1.42 (br d, J=6.4 Hz, 7H), 1.22 (br s, 40H), 0.88-0.87 (m, 1H), 0.97-0.79 (m, 18H)

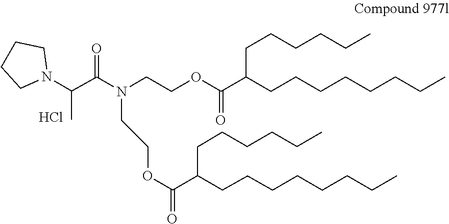 [SMILES: CCCCCCCCC(CCCCCC)C(=O)OCCN(CCOC(=O)C(CCCCCC)CCCCCCCC)C(=O)C(C)N1CCCC1 ; Cl]

Compound 977l. ¹H NMR (400 MHz, CHLOROFORM-d) δ=12.68 (br s, 1H), 4.50 (br d, J=4.3 Hz, 1H), 4.31-4.13 (m, 4H), 3.99-3.87 (m, 1H), 3.83-3.76 (m, 1H), 3.72-3.53 (m, 4H), 3.53-3.44 (m, 1H), 3.02-2.82 (m, 1H), 2.36-2.28 (m, 2H), 2.20-2.04 (m, 4H), 1.71 (d, J=6.8 Hz, 3H), 1.61-1.52 (m, 4H), 1.49-1.40 (m, 4H), 1.32-1.19 (m, 40H), 0.97-0.81 (m, 12H)

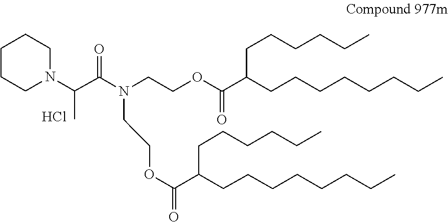 [SMILES: CCCCCCCCC(CCCCCC)C(=O)OCCN(CCOC(=O)C(CCCCCC)CCCCCCCC)C(=O)C(C)N1CCCCC1 ; Cl]

Compound 977m. ¹H NMR (400 MHz, CHLOROFORM-d) δ=11.94-11.81 (m, 1H), 4.49-4.40 (m, 1H), 4.32-4.17 (m, 4H), 3.79-3.69 (m, 2H), 3.68-3.49 (m, 4H), 3.42 (br d, J=10.8 Hz, 1H), 3.07-2.94 (m, 1H), 2.42-2.16 (m, 4H), 1.90 (br d, J=12.8 Hz, 2H), 1.80 (br s, 1H), 1.69 (d, J=6.8 Hz, 3H), 1.62-1.51 (m, 4H), 1.50-1.40 (m, 5H), 1.32-1.20 (m, 40H), 0.88 (t, J=6.8 Hz, 12H)

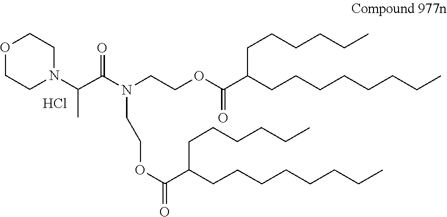 [SMILES: CCCCCCCCC(CCCCCC)C(=O)OCCN(CCOC(=O)C(CCCCCC)CCCCCCCC)C(=O)C(C)N1CCOCC1 ; Cl]

Compound 977n. ¹H NMR (400 MHz, CHLOROFORM-d) δ=13.15 (br s, 1H), 4.44 (br d, J=6.0 Hz, 1H), 4.39-4.28 (m, 2H), 4.28-4.18 (m, 4H), 4.00 (br d, J=13.2 Hz, 1H), 3.93 (br d, J=11.2 Hz, 2H), 3.82-3.67 (m, 2H), 3.65-3.51 (m, 2H), 3.48-3.32 (m, 2H), 3.26 (br s, 1H), 2.37-2.26 (m, 2H), 1.69 (br d, J=6.0 Hz, 3H), 1.63-1.51 (m, 4H), 1.45 (m, 4H), 1.34-1.15 (m, 40H), 0.88 (t, J=6.8 Hz, 12H)

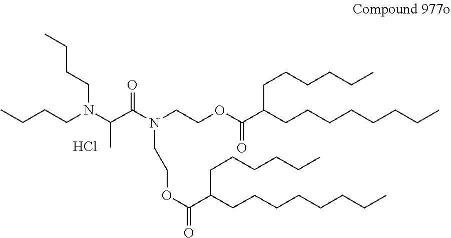 [SMILES: CCCCCCCCC(CCCCCC)C(=O)OCCN(CCOC(=O)C(CCCCCC)CCCCCCCC)C(=O)C(C)N(CCCC)CCCC ; Cl]

Compound 977o. ¹H NMR (400 MHz, CHLOROFORM-d) δ=11.83 (br s, 1H), 4.53 (br s, 1H), 4.36-4.11 (m, 4H), 3.80-3.67 (m, 2H), 3.66-3.42 (m, 3H), 3.30 (br s, 1H), 3.23-3.04 (m, 2H), 2.31 (br d, J=4.4 Hz, 2H), 2.08-1.96 (m, 1H), 1.73 (br s, 3H), 1.56 (br s, 4H), 1.50-1.35 (m, 8H), 1.34-1.06 (m, 43H), 0.97 (q, J=7.2 Hz, 6H), 0.88 (t, J=6.8 Hz, 12H)

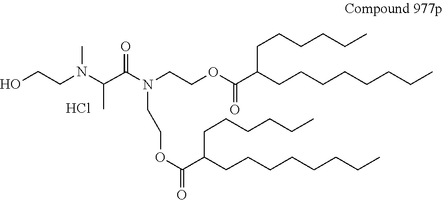 [SMILES: CCCCCCCCC(CCCCCC)C(=O)OCCN(CCOC(=O)C(CCCCCC)CCCCCCCC)C(=O)C(C)N(C)CCO ; Cl]

Compound 977p. ¹H NMR (400 MHz, DMSO-d₆) δ=10.03-9.55 (m, 1H), 5.35 (br s, 1H), 4.71-4.50 (m, 1H), 4.33-4.10 (m, 4H), 3.84-3.65 (m, 4H), 3.63-3.39 (m, 2H), 3.30-3.06 (m, 2H), 2.84 (br s, 3H), 2.37-2.23 (m, 2H), 1.53-1.35 (m, 11H), 1.22 (br s, 40H), 0.85 (t, J=6.8 Hz, 12H)

### Representative Synthesis and Characterization of Compounds 987a-p

Common intermediates: Synthesis of Compounds A-C were described above. Compounds J-L were prepared under the same reaction conditions.

### Representative Synthesis and Characterization of Common Intermediates, Compounds J-L:

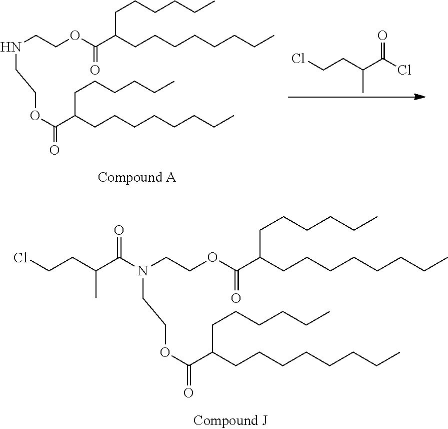

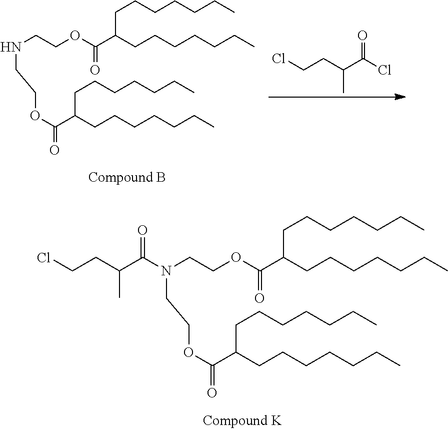

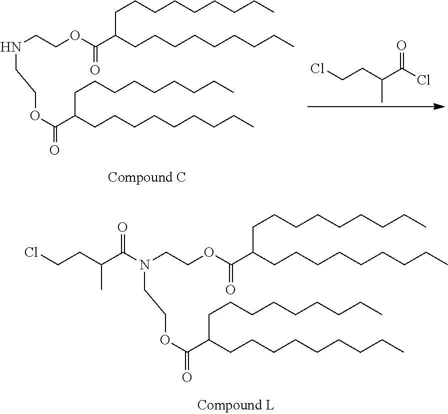

### Representative Preparation of Compounds J-L: Preparation of Compound K

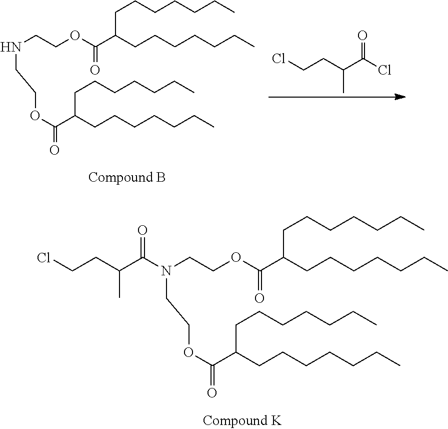

To a solution of Compound B (3.3 g, 5.67 mmol, 1 eq.) in DCM (33 mL) was added TEA (573.80 mg, 5.67 mmol, 789.27 μL, 1 eq.) and 4-chloro-2-methylbutanoyl chloride (1.32 g, 8.51 mmol, 1.5 eq) at 0° C. The mixture was stirred at 20° C. for 1 hour. TLC (Petroleum ether:Ethyl acetate=5:1, R_{f}=0.78) indicated Compound B was consumed and new spots formed. The reaction mixture was concentrated under reduced pressure to give crude product. The crude product was purified by column chromatography (SiO₂, Petroleum ether:Ethyl acetate=98:2 to 94:6) to give Compound K (3.8 g, 5.42 mmol, 76.00% yield) as a light yellow oil.

LCMS [M+23]⁺=722.5. ¹H NMR (400 MHz, CHLOROFORM-d) δ=4.28-4.17 (m, 4H), 3.82-3.58 (m, 5H), 3.54-3.45 (m, 1H), 3.14-3.05 (m, 1H), 2.39-2.16 (m, 3H), 1.90-1.76 (m, 1H), 1.58-1.54 (m, 4H), 1.52-1.38 (m, 4H), 1.37-1.20 (m, 40H), 1.18 (d, J=6.8 Hz, 3H), 0.88 (t, J=6.8 Hz, 12H)

### Compound J

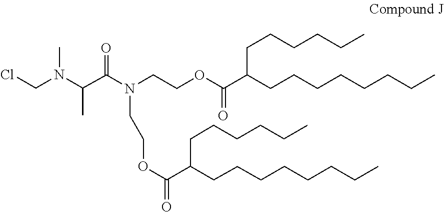 [SMILES: CCCCCCCCC(CCCCCC)C(=O)OCCN(CCOC(=O)C(CCCCCC)CCCCCCCC)C(=O)C(C)N(C)CCl]

### LCMS [M+23]⁺=722.5

¹H NMR (400 MHz, CHLOROFORM-d) δ=4.29-4.16 (m, 4H), 3.82-3.73 (m, 1H), 3.73-3.57 (m, 4H), 3.55-3.44 (m, 1H), 3.15-3.04 (m, 1H), 2.39-2.16 (m, 3H), 1.92-1.75 (m, 2H), 1.59 (m, 4H), 1.52-1.40 (m, 4H), 1.37-1.13 (m, 43H), 0.88 (t, J=6.8 Hz, 12H)

### Compound L

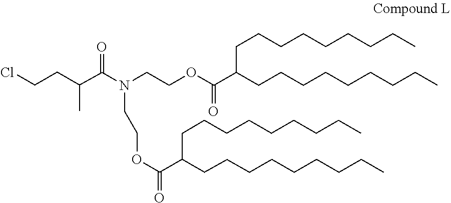 [SMILES: CCCCCCCCCC(CCCCCCCCC)C(=O)OCCN(CCOC(=O)C(CCCCCCCCC)CCCCCCCCC)C(=O)C(C)CCCl]

¹H NMR (400 MHz, CHLOROFORM-d) δ=4.30-4.15 (m, 4H), 3.83-3.71 (m, 1H), 3.70-3.55 (m, 4H), 3.54-3.44 (m, 1H), 3.16-3.02 (m, 1H), 2.40-2.27 (m, 2H), 2.25-2.14 (m, 1H), 1.83 (tdd, J=5.2, 8.8, 14.0 Hz, 1H), 1.60-1.52 (m, 4H), 1.50-1.40 (m, 4H), 1.26 (s, 56H), 1.17 (d, J=6.8 Hz, 3H), 0.88 (t, J=6.8 Hz, 12H).

Synthesis of Compounds 987a-p: Prepared in the same way from common intermediates. Compounds 987a-p were all synthesized using 20 equiv. of amine.

### Representative Synthesis and Characterization of Compound 987a

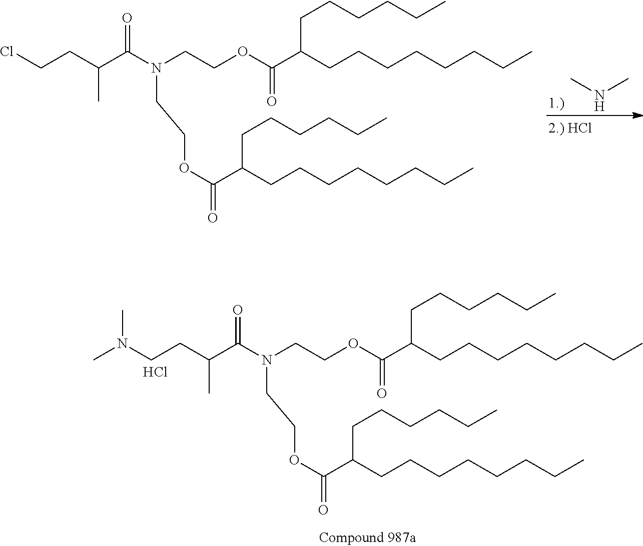

1.) To a solution of Compound J (300 mg, 428.26 μmol, 1 eq.) in dioxane (1 mL) and toluene (1 mL) was added KI (78.20 mg, 471.08 μmol, 1.1 eq.), TEA (173.34 mg, 1.71 mmol, 238.43 μL, 4 eq.) and dimethylamine (2 M, 4.28 mL, 20 eq.). The mixture was stirred at 80° C. for 6 hours. LCMS showed trace of Compound J remained and desired MS was detected. The reaction mixture was diluted with H₂O (10 mL) and extracted with DCM (60 mL). The combined organic layers were washed with brine (20 mL), dried over Na₂SO₄, filtered and concentrated under reduced pressure to give a residue. The residue was purified by column chromatography (SiO₂, Dichloromethane:Methanol=200:1 to 70:1) to give a colorless oil (80 mg, 112.81 μmol, 26.34% yield, 100% purity).

LCMS [M+1]⁺=709.6. ¹H NMR (400 MHz, CHLOROFORM-d) δ=4.21 (t, J=6.4 Hz, 4H), 3.89-3.74 (m, 1H), 3.68-3.50 (m, 3H), 2.86 (br dd, J=6.8, 13.6 Hz, 1H), 2.45-2.29 (m, 4H), 2.27 (s, 6H), 1.92 (dt, J=8.4, 13.6 Hz, 2H), 1.67-1.53 (m, 4H), 1.53-1.37 (m, 4H), 1.26 (br s, 40H), 1.15 (d, J=6.8 Hz, 3H), 0.88 (t, J=6.8 Hz, 12H) 2.) A portion of this oil (70 mg, 98.71 μmol, 1 eq.) was dissolved in HCl/H₂O (14.10 μmol, 20 mL, 0.04 wt %) and stirred at 25° C. for 1 hour. The mixture was lyophilized to give Compound 987a (29.51 mg, 39.58 μmol, 40.10% yield, HCl) as a colorless gum.

¹H NMR (400 MHz, CHLOROFORM-d) δ=12.34 (br s, 1H), 4.29 (td, J=5.2, 11.2 Hz, 2H), 4.20-4.05 (m, 2H), 3.83-3.71 (m, 1H), 3.63 (br t, J=5.6 Hz, 2H), 3.53-3.34 (m, 1H), 3.04 (m, 1H), 2.94 (m, 2H), 2.80 (br t, J=4.0 Hz, 6H), 2.40-2.27 (m, 2H), 2.23 (br dd, J=3.2, 4.8 Hz, 1H), 2.04 (br d, J=1.2 Hz, 1H), 1.63-1.52 (m, 4H), 1.46 (m, 4H), 1.35-1.17 (m, 43H), 0.88 (t, J=6.8 Hz, 12H)

### Characterization of Compounds 987b-p

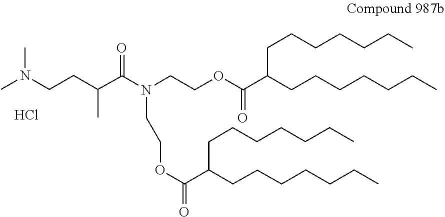 [SMILES: CCCCCCCC(CCCCCCC)C(=O)OCCN(CCOC(=O)C(CCCCCCC)CCCCCCC)C(=O)C(C)CCN(C)C ; Cl]

987b. ¹H NMR (400 MHz, CHLOROFORM-d) δ=12.80-11.89 (m, 1H), 4.30 (td, J=5.6, 11.2 Hz, 2H), 4.12 (qd, J=5.6, 11.2 Hz, 2H), 3.76 (td, J=5.6, 14.0 Hz, 1H), 3.63 (br t, J=6.4 Hz, 2H), 3.44 (td, J=5.6, 14.0 Hz, 1H), 3.13-2.87 (m, 3H), 2.80 (s, 6H), 2.38-2.15 (m, 3H), 2.12-1.98 (m, 1H), 1.57 (m, 4H), 1.51-1.40 (m, 4H), 1.37-1.16 (m, 43H), 0.88 (t, J=6.8 Hz, 12H)

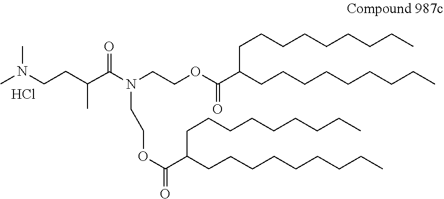 [SMILES: CCCCCCCCCC(CCCCCCCCC)C(=O)OCCN(CCOC(=O)C(CCCCCCCCC)CCCCCCCCC)C(=O)C(C)CCN(C)C ; Cl]

Compound 987c. ¹H NMR (400 MHz, CHLOROFORM-d) δ=12.89-12.07 (m, 1H), 4.36-4.24 (m, 2H), 4.18-4.06 (m, 2H), 3.76 (td, J=5.6, 14.0 Hz, 1H), 3.63 (br t, J=6.4 Hz, 2H), 3.52-3.37 (m, 1H), 3.12-2.85 (m, 3H), 2.79 (s, 6H), 2.40-2.15 (m, 3H), 2.12-1.95 (m, 1H), 1.60-1.51 (m, 4H), 1.50-1.39 (m, 4H), 1.32-1.20 (m, 59H), 0.88 (t, J=6.9 Hz, 12H).

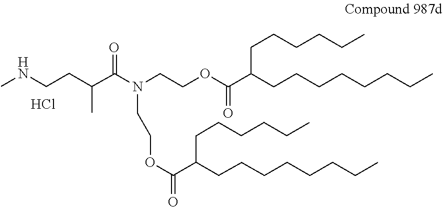 [SMILES: CCCCCCCCC(CCCCCC)C(=O)OCCN(CCOC(=O)C(CCCCCC)CCCCCCCC)C(=O)C(C)CCNC ; Cl]

Compound 987d. ¹H NMR (400 MHz, CHLOROFORM-d) δ=9.87-9.30 (m, 2H), 4.43-4.04 (m, 4H), 3.92-3.76 (m, 1H), 3.72-3.56 (m, 2H), 3.48-3.30 (m, 1H), 3.04 (br s, 3H), 2.71 (br s, 3H), 2.42-2.26 (m, 2H), 2.23-1.98 (m, 2H), 1.65-1.51 (m, 4H), 1.46 (m, 4H), 1.38-1.03 (m, 43H), 0.88 (t, J=6.8 Hz, 12H)

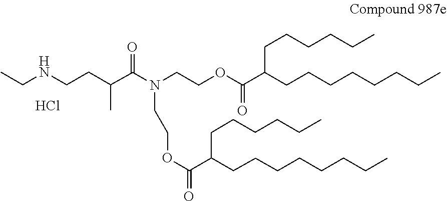 [SMILES: CCCCCCCCC(CCCCCC)C(=O)OCCN(CCOC(=O)C(CCCCCC)CCCCCCCC)C(=O)C(C)CCNCC ; Cl]

Compound 987e. ¹H NMR (400 MHz, CHLOROFORM-d) δ=10.01-9.70 (m, 1H), 9.46-9.19 (m, 1H), 4.37-4.09 (m, 4H), 3.93-3.78 (m, 1H), 3.64 (q, J=6.0 Hz, 2H), 3.46-3.28 (m, 1H), 3.18-2.92 (m, 5H), 2.33 (tdd, J=2.8, 5.6, 8.4 Hz, 2H), 2.23-2.00 (m, 2H), 1.52-1.40 (m, 8H), 1.37-1.15 (m, 46H), 0.89 (t, J=6.8 Hz, 12H)

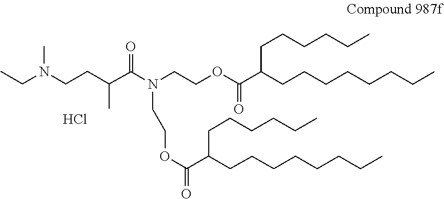 [SMILES: CCCCCCCCC(CCCCCC)C(=O)OCCN(CCOC(=O)C(CCCCCC)CCCCCCCC)C(=O)C(C)CCN(C)CC ; Cl]

Compound 987f. ¹H NMR (400 MHz, CHLOROFORM-d) δ=12.20-11.98 (m, 1H), 4.36-4.22 (m, 2H), 4.19-4.05 (m, 2H), 3.78 (br dd, J=6.4, 13.6 Hz, 1H), 3.62 (br t, J=6.0 Hz, 2H), 3.42 (br dd, J=6.0, 13.6 Hz, 1H), 3.29-2.85 (m, 5H), 2.75 (dd, J=5.2, 6.8 Hz, 3H), 2.33 (dt, J=3.2, 8.4 Hz, 2H), 2.22 (br d, J=7.2 Hz, 1H), 2.11-1.94 (m, 1H), 1.61-1.53 (m, 4H), 1.51-1.39 (m, 8H), 1.37-1.15 (m, 43H), 0.88 (t, J=6.8 Hz, 12H)

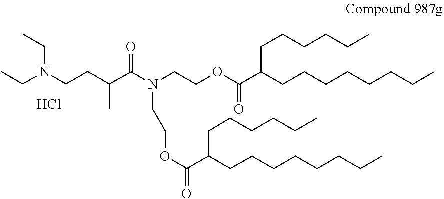 [SMILES: CCCCCCCCC(CCCCCC)C(=O)OCCN(CCOC(=O)C(CCCCCC)CCCCCCCC)C(=O)C(C)CCN(CC)CC ; Cl]

Compound 987g. ¹H NMR (400 MHz, CHLOROFORM-d) δ=10.99-10.21 (m, 1H), 4.28 (tt, J=5.6, 10.8 Hz, 2H), 4.14 (qd, J=5.6, 11.6 Hz, 2H), 3.81 (td, J=5.6, 14.0 Hz, 1H), 3.72-3.57 (m, 2H), 3.46-3.35 (m, 1H), 3.31-3.07 (m, 5H), 3.06-2.88 (m, 2H), 2.39-2.29 (m, 2H), 2.26-2.04 (m, 2H), 1.58-1.52 (m, 4H), 1.46 (br d, J=6.4 Hz, 10H), 1.37-1.16 (m, 43H), 0.89 (t, J=6.8 Hz, 12H)

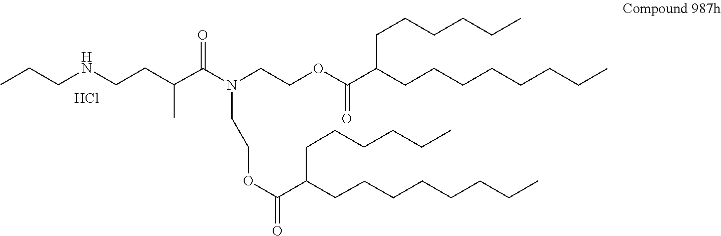 [SMILES: CCCCCCCCC(CCCCCC)C(=O)OCCN(CCOC(=O)C(CCCCCC)CCCCCCCC)C(=O)C(C)CCNCCC ; Cl]

Compound 987h. ¹H NMR (400 MHz, CHLOROFORM-d) δ=9.76 (br s, 1H), 9.41 (br s, 1H), 4.34-4.11 (m, 4H), 3.89-3.77 (m, 1H), 3.70-3.56 (m, 2H), 3.44-3.33 (m, 1H), 3.13-2.81 (m, 5H), 2.38-2.27 (m, 2H), 2.13 (br d, J=4.8 Hz, 2H), 1.96-1.85 (m, 2H), 1.64-1.52 (m, 4H), 1.46 (m, 4H), 1.36-1.16 (m, 43H), 1.03 (t, J=7.2 Hz, 3H), 0.88 (t, J=6.8 Hz, 12H)

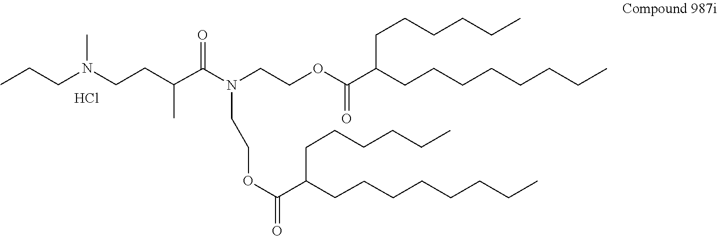 [SMILES: CCCCCCCCC(CCCCCC)C(=O)OCCN(CCOC(=O)C(CCCCCC)CCCCCCCC)C(=O)C(C)CCN(C)CCC ; Cl]

Compound 987i. ¹H NMR (400 MHz, CHLOROFORM-d) δ=12.17 (s, 1H), 4.36-4.21 (m, 2H), 4.13 (d, J=5.2, 10.8 Hz, 2H), 3.82-3.74 (m, 1H), 3.62 (t, J=6.0 Hz, 2H), 3.42 (d, J=5.6, 13.6 Hz, 1H), 3.14-2.83 (m, 5H), 2.76 (t, J=5.6 Hz, 3H), 2.40-2.29 (m, 2H), 2.22 (d, J=3.2, 8.4 Hz, 1H), 2.11-1.99 (m, 1H), 1.97-1.81 (m, 2H), 1.58-1.40 (m, 8H), 1.35-1.17 (m, 43H), 1.02 (td, J=7.2, 10.4 Hz, 3H), 0.89 (t, J=6.8 Hz, 12H)

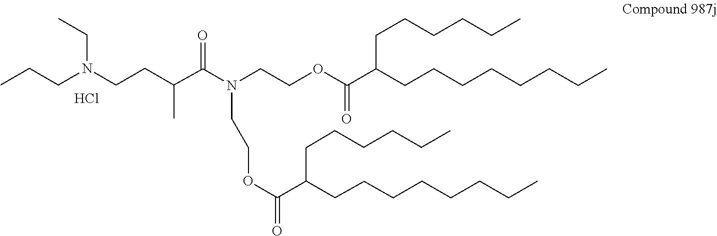 [SMILES: CCCCCCCCC(CCCCCC)C(=O)OCCN(CCOC(=O)C(CCCCCC)CCCCCCCC)C(=O)C(C)CCN(CC)CCC ; Cl]

Compound 987j. ¹H NMR (400 MHz, CHLOROFORM-d) δ=11.98 (br s, 1H), 4.26 (td, J=5.2, 10.4 Hz, 2H), 4.14 (br dd, J=6.0, 10.8 Hz, 2H), 3.90-3.74 (m, 1H), 3.62 (br t, J=6.0 Hz, 2H), 3.49-3.35 (m, 1H), 3.25-3.06 (m, 2H), 3.05-2.76 (m, 5H), 2.33 (br dd, J=4.0, 8.4 Hz, 2H), 2.26-2.11 (m, 1H), 2.09-1.96 (m, 1H), 1.95-1.77 (m, 2H), 1.61-1.51 (m, 4H), 1.51-1.37 (m, 7H), 1.35-1.17 (m, 43H), 1.07-0.97 (m, 3H), 0.88 (t, J=6.8 Hz, 12H)

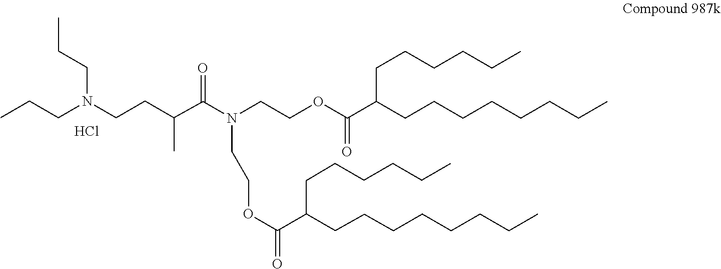 [SMILES: CCCCCCCCC(CCCCCC)C(=O)OCCN(CCOC(=O)C(CCCCCC)CCCCCCCC)C(=O)C(C)CCN(CCC)CCC ; Cl]

Compound 987k. ¹H NMR (400 MHz, CHLOROFORM-d) δ=12.04-11.91 (m, 1H), 4.33-4.21 (m, 2H), 4.19-4.06 (m, 2H), 3.80 (td, J=5.2, 14.0 Hz, 1H), 3.61 (br t, J=6.4 Hz, 2H), 3.46-3.34 (m, 1H), 3.07-2.83 (m, 7H), 2.39-2.28 (m, 2H), 2.25-2.11 (m, 1H), 2.10-1.97 (m, 1H), 1.87 (td, J=8.0, 16.4 Hz, 4H), 1.57 (br dd, J=5.2, 13.2 Hz, 4H), 1.52-1.41 (m, 4H), 1.34-1.17 (m, 43H), 1.01 (td, J=7.6, 9.6 Hz, 6H), 0.88 (t, J=6.8 Hz, 12H)

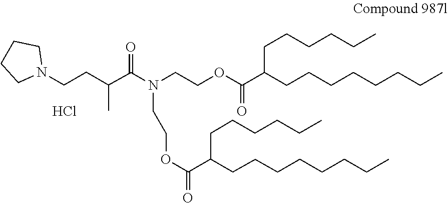 [SMILES: CCCCCCCCC(CCCCCC)C(=O)OCCN(CCOC(=O)C(CCCCCC)CCCCCCCC)C(=O)C(C)CCN1CCCC1 ; Cl]

Compound 987l. ¹H NMR (400 MHz, CHLOROFORM-d) δ=12.31 (s, 1H), 4.36-4.22 (m, 2H), 4.17-4.06 (m, 2H), 3.84-3.69 (m, 3H), 3.63 (t, J=6.0 Hz, 2H), 3.46 (td, J=5.6, 13.9 Hz, 1H), 3.19-3.07 (m, 1H), 3.00-2.82 (m, 3H), 2.78 (m, 1H), 2.39-2.28 (m, 2H), 2.22 (d, J=6.4 Hz, 3H), 2.16-1.98 (m, 3H), 1.46 (m, 8H), 1.36-1.16 (m, 43H), 0.89 (t, J=6.8 Hz, 12H)

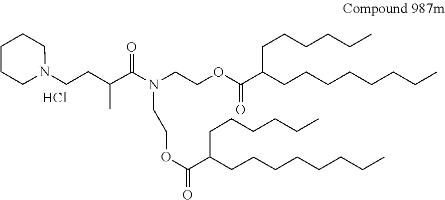 [SMILES: CCCCCCCCC(CCCCCC)C(=O)OCCN(CCOC(=O)C(CCCCCC)CCCCCCCC)C(=O)C(C)CCN1CCCCC1 ; Cl]

Compound 987m. ¹H NMR (400 MHz, CHLOROFORM-d) δ=11.88 (m, 1H), 4.34-4.22 (m, 2H), 4.13 (td, J=5.6, 11.6 Hz, 2H), 3.82-3.70 (m, 1H), 3.67-3.55 (m, 3H), 3.52-3.37 (m, 2H), 3.05-2.80 (m, 3H), 2.71-2.54 (m, 2H), 2.41-2.17 (m, 5H), 2.13 (m, 1H), 1.95-1.77 (m, 3H), 1.62-1.52 (m, 4H), 1.51-1.38 (m, 5H), 1.33-1.17 (m, 43H), 0.88 (t, J=6.8 Hz, 12H)

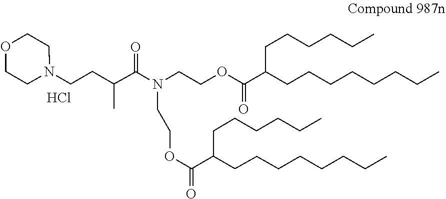 [SMILES: CCCCCCCCC(CCCCCC)C(=O)OCCN(CCOC(=O)C(CCCCCC)CCCCCCCC)C(=O)C(C)CCN1CCOCC1 ; Cl]

Compound 987n. ¹H NMR (400 MHz, CHLOROFORM-d) δ=13.04-12.83 (m, 1H), 4.39-4.22 (m, 4H), 4.11 (qd, J=6.0, 12.4 Hz, 2H), 4.03-3.90 (m, 2H), 3.84-3.69 (m, 1H), 3.62 (br t, J=6.0 Hz, 2H), 3.56-3.33 (m, 3H), 3.12-2.78 (m, 5H), 2.40-2.21 (m, 3H), 2.19-2.06 (m, 1H), 1.60-1.52 (m, 4H), 1.52-1.39 (m, 4H), 1.34-1.19 (m, 43H), 0.89 (t, J=6.8 Hz, 12H).

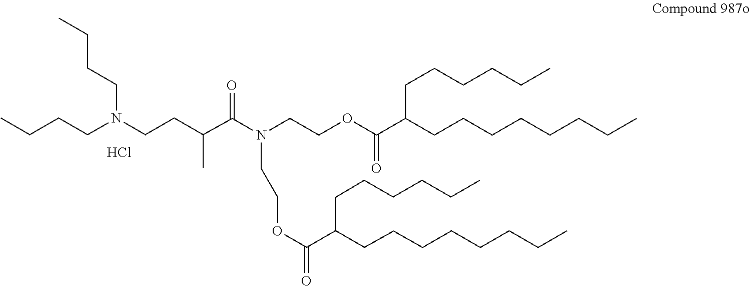 [SMILES: CCCCCCCCC(CCCCCC)C(=O)OCCN(CCOC(=O)C(CCCCCC)CCCCCCCC)C(=O)C(C)CCN(CCCC)CCCC ; Cl]

Compound 987o. ¹H NMR (400 MHz, CHLOROFORM-d) δ=11.88-11.09 (m, 1H), 4.34-4.21 (m, 2H), 4.15 (dt, J=5.6, 10.8 Hz, 2H), 3.86-3.74 (m, 1H), 3.70-3.58 (m, 2H), 3.47-3.36 (m, 1H), 3.13-2.87 (m, 7H), 2.38-2.28 (m, 2H), 2.26-1.99 (m, 2H), 1.80 (M, 4H), 1.50-1.38 (m, 8H), 1.37-1.18 (m, 47H), 1.06-0.95 (m, 6H), 0.89 (t, J=6.8 Hz, 12H)

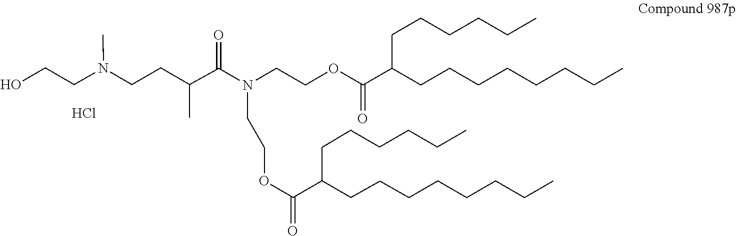 [SMILES: CCCCCCCCC(CCCCCC)C(=O)OCCN(CCOC(=O)C(CCCCCC)CCCCCCCC)C(=O)C(C)CCN(C)CCO ; Cl]

Compound 987p. ¹H NMR (400 MHz, DMSO-d6) δ=9.51-9.35 (m, 1H), 5.39-5.25 (m, 1H), 4.22-4.02 (m, 4H), 3.70 (m, 2H), 3.67-3.55 (m, 3H), 3.45-3.35 (m, 2H), 3.21-3.12 (m, 1H), 3.12-2.95 (m, 2H), 2.86-2.79 (m, 1H), 2.74 (br s, 3H), 2.35-2.22 (m, 2H), 1.97-1.83 (m, 1H), 1.78-1.61 (m, 1H), 1.54-1.44 (m, 4H), 1.44-1.34 (m, 4H), 1.22 (br s, 41H), 1.04 (d, J=6.8 Hz, 3H), 0.84 (t, J=6.4 Hz, 12H).

### General Procedures for the Preparation of Series 900 Lipidoids 917a Through 917i

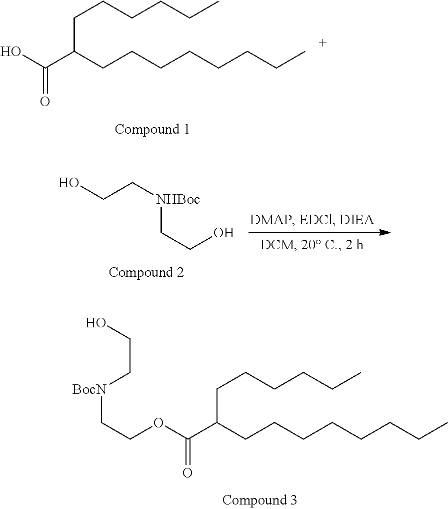

To a solution of Compound 1 (9.37 g, 36.54 mmol, 1 eq.) in DCM (75 mL) was added DMAP (446.41 mg, 3.65 mmol, 0.1 eq.), EDCI (8.41 g, 43.85 mmol, 1.2 eq.), DIEA (7.08 g, 54.81 mmol, 9.55 mL, 1.5 eq.) and Compound 2 (7.5 g, 36.54 mmol, 6.91 mL, 1 eq.). The mixture was stirred at 20° C. for 12 hours. LCMS indicated Compound 1 was consumed completely and one main peak with desired MS was detected. The reaction mixture was diluted with H₂O (100 mL) and extracted with DCM (125 mL×2). The combined organic layers were dried over Na₂SO₄, filtered and concentrated under reduced pressure to give a residue. The residue was purified by column chromatography (SiO₂, petroleum ether:ethyl acetate=70:1 to 50:1) to give Compound 3 (8.96 g, 20.20 mmol, 27.63% yield) as a colorless oil. (1 Batch: 7.5 g, 2 Batches) LCMS [M−100+1]⁺=344.4

¹H NMR (400 MHz, CHLOROFORM-d) δ=4.19 (br s, 2H), 3.77 (br s, 2H), 3.48 (br s, 4H), 2.38-2.29 (m, 1H), 1.64-1.54 (m, 2H), 1.48 (s, 9H), 1.46-1.39 (m, 2H), 1.33-1.20 (m, 20H), 0.88 (t, J=6.8 Hz, 6H).

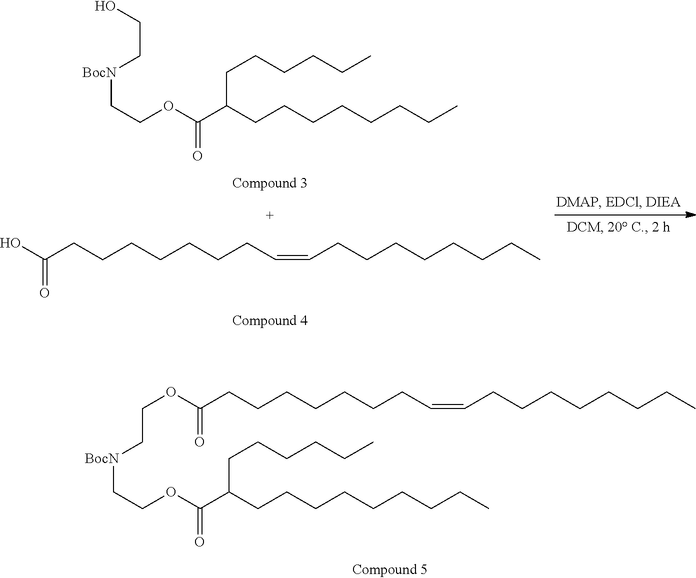 [SMILES: CCCCCCCCC(CCCCCC)C(=O)OCCN(CCO)C(=O)OC(C)(C)C ; CCCCCCCC/C=C\CCCCCCCC(=O)O ; CCCCCCCC/C=C\CCCCCCCC(=O)OCCN(CCOC(=O)C(CCCCCC)CCCCCCCCC)C(=O)OC(C)(C)C]

To a solution of Compound 4 (6.72 g, 23.80 mmol, 6.72 mL, 1.2 eq.) in DCM (90 mL) was added DMAP (242.32 mg, 1.98 mmol, 0.1 eq.), EDCI (4.56 g, 23.80 mmol, 1.2 eq.), DIEA (3.85 g, 29.75 mmol, 5.18 mL, 1.5 eq.) and Compound 3 (8.8 g, 19.84 mmol, 1 eq.). The mixture was stirred at 20° C. for 2 hours. TLC (petroleum ether:ethyl acetate=5:1, R_{f}=0.50) indicated Compound 3 was consumed completely and one new spot formed. The mixture was concentrated under reduced pressure to give a residue. The residue was purified by column chromatography (SiO₂, petroleum ether:ethyl acetate=92:8 to 95:5) to give Compound 5 (13.47 g, 19.02 mmol, 95.90% yield) as a yellow oil.

LCMS [M−100+1]⁺=608.6

¹H NMR (400 MHz, CHLOROFORM-d) δ=5.43-5.29 (m, 2H), 4.27-4.11 (m, 4H), 3.61-3.40 (m, 4H), 2.40-2.26 (m, 3H), 2.07-1.94 (m, 4H), 1.67-1.57 (m, 4H), 1.47 (s, 9H), 1.46-1.41 (m, 2H), 1.38-1.20 (m, 40H), 0.96-0.81 (m, 9H).

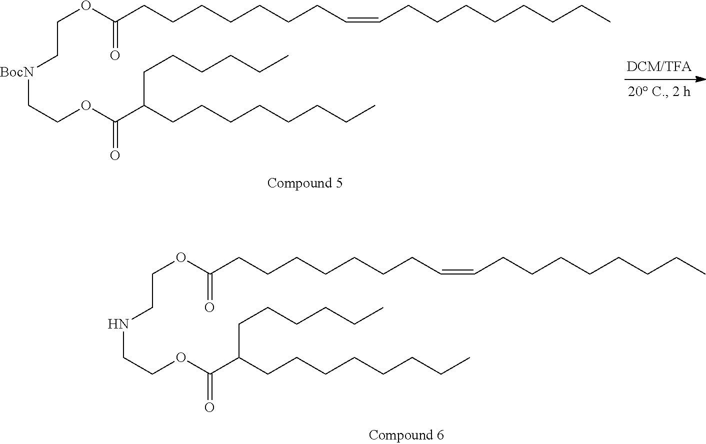 [SMILES: CCCCCCCC/C=C\CCCCCCCC(=O)OCCN(CCOC(=O)C(CCCCCC)CCCCCCCC)C(=O)OC(C)(C)C ; CCCCCCCC/C=C\CCCCCCCC(=O)OCCNCCOC(=O)C(CCCCCC)CCCCCCCC]

To a solution of Compound 5 (7.4 g, 10.45 mmol, 1 eq) in DCM (70 mL) was added TFA (14 mL). The mixture was stirred at 20° C. for 2 hours. TLC (petroleum ether:ethyl acetate=5:1, R_{f}=0) indicated Compound 5 was consumed completely and one new spot formed. The reaction mixture was concentrated under reduced pressure to give Compound 6 (7.5 g, 10.39 mmol, 99.40% yield, TFA) as a yellow oil.

LCMS [M+1]⁺=608.6

¹H NMR (400 MHz, CHLOROFORM-d) δ=5.43-5.27 (m, 2H), 4.40 (td, J=2.8, 6.8 Hz, 4H), 3.42 (br d, J=1.2 Hz, 4H), 2.48-2.29 (m, 3H), 2.02 (q, J=6.0 Hz, 4H), 1.63-1.41 (m, 6H), 1.35-1.20 (m, 40H), 0.89 (t, J=6.8 Hz, 9H).

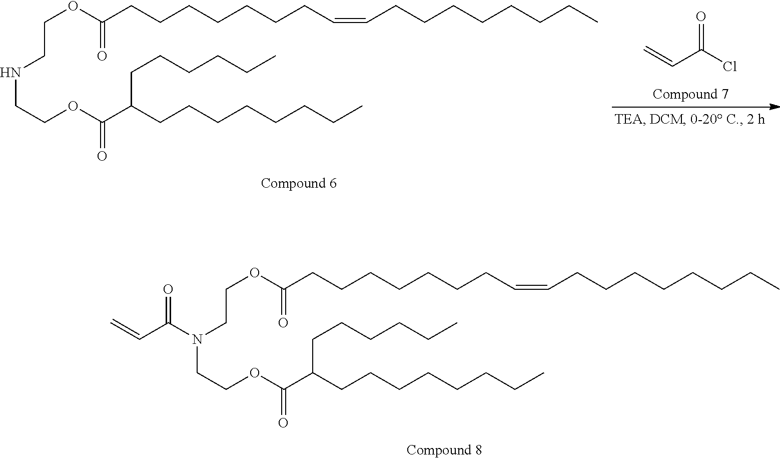

To a solution of Compound 6 (7.4 g, 12.17 mmol, 1 eq.) in DCM (74 mL) was added TEA (3.69 g, 36.51 mmol, 5.08 mL, 3 eq.) at 20° C., followed by Compound 7 (1.65 g, 18.26 mmol, 1.48 mL, 1.5 eq) at 0° C. The mixture was stirred at 20° C. for 2 hours. TLC (petroleum ether:ethyl acetate=5:1, R_{f}=0.35) indicated Compound 6 was consumed completely and one new spot formed. The reaction mixture was concentrated under reduced pressure to give a residue. The residue was purified by column chromatography (SiO₂, petroleum ether:ethyl acetate=50:1 to 20:1) to give Intermediate Compound 8 (5.8 g, 8.76 mmol, 71.98% yield) as a yellow oil.

LCMS [M+1]⁺=662.5

¹H NMR (400 MHz, CHLOROFORM-d) δ=6.70-6.59 (m, 1H), 6.40 (dd, J=2.0, 16.8 Hz, 1H), 5.74 (br d, J=10.4 Hz, 1H), 5.42-5.29 (m, 2H), 4.29-4.19 (m, 4H), 3.72-3.66 (m, 4H), 2.35-2.27 (m, 3H), 2.02 (q, J=6.4 Hz, 4H), 1.66-1.60 (m, 2H), 1.55 (br s, 2H), 1.48-1.41 (m, 2H), 1.35-1.22 (m, 40H), 0.89 (t, J=6.8 Hz, 9H).

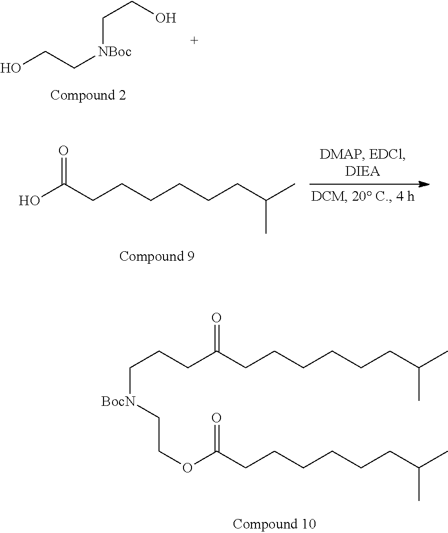 [SMILES: CC(C)(C)OC(=O)N(CCO)CCO ; CC(C)CCCCCCC(=O)O ; CC(C)CCCCCCC(=O)CCCN(CCOC(=O)CCCCCCC(C)C)C(=O)OC(C)(C)C]

A mixture of Compound 9 (9.06 g, 52.62 mmol, 2.4 eq.), Compound 2 (4.5 g, 21.92 mmol, 4.15 mL, 1 eq.) EDCI (10.51 g, 54.81 mmol, 2.5 eq.), DIEA (8.50 g, 65.77 mmol, 11.46 mL, 3 eq.) and DMAP (535.69 mg, 4.38 mmol, 0.2 eq.) in DCM (45 mL) was degassed and purged with N₂ three times, and the mixture was stirred at 20° C. for 4 hours under N₂ atmosphere. TLC (petroleum ether:ethyl acetate=3:1, R_{f}=0.60) indicated Compound 2 was consumed, and one new spot was detected. The reaction mixture was diluted with saturated NH₄Cl (10 mL) and extracted with DCM (20 mL*2). The combined organic layers were washed with brine (20 mL), dried over Na₂SO₄, filtered and concentrated under reduced pressure to give a residue.

The residue was purified by column chromatography (SiO₂, petroleum ether:ethyl acetate=50:1 to 15:1) to give Compound 10 (10.35 g, 20.06 mmol, 91.48% yield, 99.56% purity) as white solid.

LCMS [M−100+1]⁺=414.4

¹H NMR (400 MHz, CHLOROFORM-d) δ=4.25-4.11 (m, 4H), 3.56-3.43 (m, 4H), 2.30 (t, J=7.6 Hz, 4H), 1.67-1.58 (m, 4H), 1.56-1.49 (m, 2H), 1.46 (s, 9H), 1.28 (br s, 12H), 1.20-1.11 (m, 4H), 0.86 (d, J=6.8 Hz, 12H)

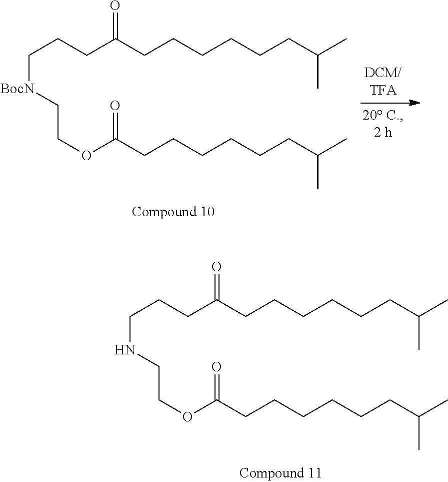 [SMILES: CC(C)CCCCCCC(=O)CCCN(CCOC(=O)CCCCCCC(C)C)C(=O)OC(C)(C)C ; CC(C)CCCCCCC(=O)CCCNCCOC(=O)CCCCCCC(C)C]

A mixture of Compound 10 (13.4 g, 26.08 mmol, 1 eq.) in DCM (111.5 mL) and TFA (22.3 mL) was degassed and purged with N₂ three times, and the mixture was stirred at 20° C. for 2 hours under N₂ atmosphere. TLC (petroleum ether:ethyl acetate=3:1, R_{f}=0.54) indicated Compound 10 was consumed, and one new spot was detected. The reaction mixture was concentrated to give Compound 11 (13.5 g, 25.48 mmol, 97.68% yield, 99.58% purity, TFA) as a yellow oil.

LCMS [M+1]⁺=414.4

¹H NMR (400 MHz, CHLOROFORM-d) δ=8.53 (br s, 2H), 4.46-4.32 (m, 4H), 3.47 (br s, 4H), 2.36 (t, J=7.6 Hz, 4H), 1.64-1.47 (m, 6H), 1.34-1.24 (m, 12H), 1.15 (q, J=6.8 Hz, 4H), 0.87 (d, J=6.8 Hz, 12H)

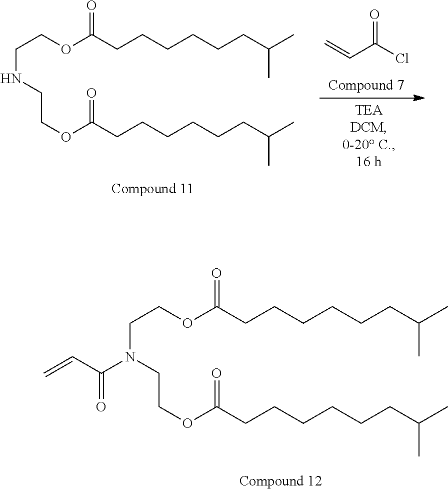

To a solution of Compound 11 (14.5 g, 27.48 mmol, 1 eq., TFA), TEA (13.90 g, 137.40 mmol, 19.12 mL, 5 eq.) in DCM (200 mL) was added Compound 7 (3.73 g, 41.22 mmol, 3.35 mL, 1.5 eq.) dropwise at 0° C. The resulting mixture was stirred at 20° C. for 16 hours. TLC (petroleum ether:ethyl acetate=3:1, R_{f}=0.62) indicated Compound 11 was consumed, and one new spot was detected. The reaction mixture was diluted with saturated NH₄Cl (50 mL) and extracted with DCM (100 mL×2). The reaction mixture was washed with brine (50 mL×2), dried over Na₂SO₄, filtered and concentrated under reduced pressure to give a residue. The residue was purified by column chromatography (SiO₂, petroleum ether:ethyl acetate=10:1 to 5:1) to give Intermediate Compound 12 (5.01 g, 10.71 mmol, 38.98% yield, 100% purity) as a yellow oil.

LCMS [M+1]⁺=468.4

¹H NMR (400 MHz, CHLOROFORM-d) δ=6.62 (dd, J=10.4, 16.8 Hz, 1H), 6.38 (dd, J=2.0, 16.8 Hz, 1H), 5.73 (dd, J=2.0, 10.4 Hz, 1H), 4.31-4.17 (m, 4H), 3.68 (t, J=6.0 Hz, 4H), 2.30 (dt, J=4.8, 7.6 Hz, 4H), 1.65-1.45 (m, 6H), 1.33-1.23 (m, 12H), 1.15 (q, J=6.8 Hz, 4H), 0.86 (d, J=6.8 Hz, 12H)

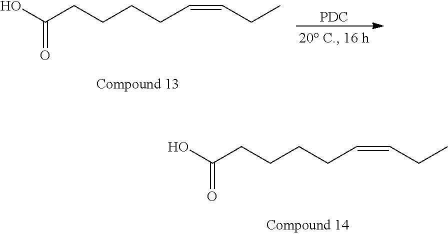

To a solution of Compound 13 (4 g, 28.12 mmol, 1 eq.) in DMF (40 mL) was added PDC (52.90 g, 140.61 mmol, 5 eq.). The mixture was stirred at 20° C. for 16 hours. TLC (petroleum ether:ethyl acetate=3:1, R_{f}=0.49) indicated Compound 13 was consumed and one major new spot was detected. The suspension was filtered through a Celite pad and the filter cake was washed with ethyl acetate (50 mL) and H₂O (50 mL). The filtrate was diluted with H₂O (300 mL) and extracted with ethyl acetate (100 mL×3), dried over Na₂SO₄, filtered and concentrated under reduced pressure to give a residue. The residue was purified by column chromatography (SiO₂, petroleum ether:ethyl acetate=100:0 to 95:5) to give Compound 14 (5.14 g, 32.90 mmol, 58.50% yield) as a colorless oil. (2 Batches)

¹H NMR (400 MHz, DMSO-d6) δ=11.98 (br s, 1H), 5.41-5.22 (m, 2H), 2.19 (t, J=7.6 Hz, 2H), 2.05-1.94 (m, 4H), 1.49 (quin, J=7.6 Hz, 2H), 1.37-1.25 (m, 2H), 0.91 (t, J=7.6 Hz, 3H)

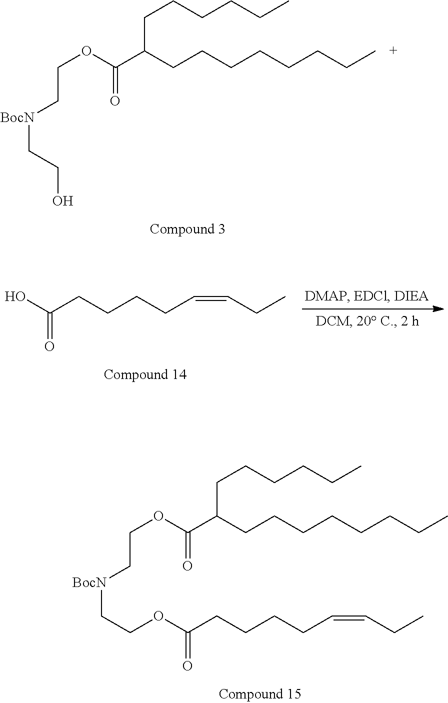 [SMILES: CCCCCCCCC(CCCCCC)C(=O)OCCN(CCO)C(=O)OC(C)(C)C ; CC/C=C\CCCCC(=O)O ; CC/C=C\CCCCC(=O)OCCN(CCOC(=O)C(CCCCCC)CCCCCCCC)C(=O)OC(C)(C)C]

To a solution of Compound 14 (2.46 g, 15.78 mmol, 1.4 eq.) in DCM (50 mL) was added EDCI (2.59 g, 13.52 mmol, 1.2 eq.), DIEA (2.18 g, 16.90 mmol, 2.94 mL, 1.5 eq.), DMAP (137.68 mg, 1.13 mmol, 0.1 eq.) and Compound 3 (5 g, 11.27 mmol, 1 eq.). The mixture was stirred at 20° C. for 2 hours. LCMS showed Compound 14 was consumed completely, and one main peak with desired MS was detected. The reaction mixture was diluted with H₂O (100 mL) and extracted with DCM (30 mL×3), dried over Na₂SO₄, filtered and concentrated under reduced pressure to give a residue. The residue was purified by column chromatography (SiO₂, petroleum ether:ethyl acetate=100:0 to 96:4) to give Compound 15 (9.45 g, 16.24 mmol, 72.05% yield) as a colorless oil. (2 Batches)

LCMS [M−100+1]⁺=482.5

¹H NMR (400 MHz, CHLOROFORM-d) δ=5.45-5.25 (m, 2H), 4.25-4.12 (m, 4H), 3.58-3.41 (m, 4H), 2.38-2.26 (m, 3H), 2.11-1.98 (m, 4H), 1.70-1.53 (m, 5H), 1.47 (s, 9H), 1.39 (br dd, J=7.6, 15.2 Hz, 3H), 1.33-1.21 (m, 20H), 0.96 (t, J=7.6 Hz, 3H), 0.88 (t, J=6.8 Hz, 6H)

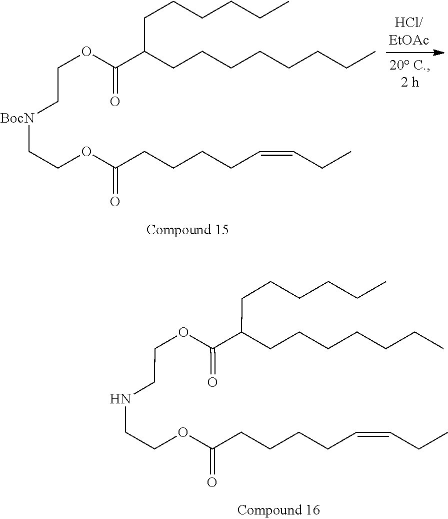 [SMILES: CC/C=C\CCCCC(=O)OCCN(CCOC(=O)C(CCCCCC)CCCCCCCC)C(=O)OC(C)(C)C ; CC/C=C\CCCCC(=O)OCCNCCOC(=O)C(CCCCCC)CCCCCCC]

A solution of Compound 15 (8.83 g, 15.18 mmol, 1 eq) in HCl/EtOAc (88 mL) was stirred at 20° C. for 2 hours. TLC (petroleum ether:ethyl acetate=3:1, R_{f}=0) indicated Compound 15 was consumed completely and a new spot formed. The reaction mixture was concentrated to give crude product. The crude product was diluted with saturated NaHCO₃ (100 mL) and extracted with DCM (30 mL×3). The combined organic layers were dried over Na₂SO₄, filtered and concentrated under reduced pressure to give Compound 16 (7.18 g, 14.90 mmol, 98.26% yield) as a colorless oil.

LCMS [M+1]⁺=482.4

¹H NMR (400 MHz, CHLOROFORM-d) δ=5.44-5.26 (m, 2H), 4.20 (q, J=5.6 Hz, 4H), 2.92 (t, J=5.6 Hz, 4H), 2.39-2.30 (m, 3H), 2.10-1.99 (m, 4H), 1.70-1.53 (m, 4H), 1.50-1.35 (m, 4H), 1.32-1.22 (m, 20H), 0.96 (t, J=7.6 Hz, 3H), 0.88 (t, J=6.8 Hz, 6H)

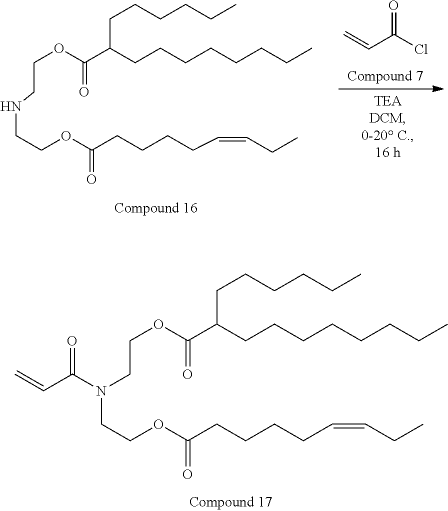

To a solution of Compound 16 (3.75 g, 7.78 mmol, 1 eq.) and TEA (2.36 g, 23.35 mmol, 3.25 mL, 3 eq.) in DCM (37.5 mL) was added Compound 7 (1.06 g, 11.68 mmol, 948.65 μL, 1.5 eq.) dropwise at 0° C.; the mixture was stirred at 20° C. for 16 hours under N₂ atmosphere. LCMS showed Compound 16 was consumed completely and one main peak with desired MS was detected. The reaction mixture was diluted with H₂O (200 mL) and extracted with DCM (100 mL×2). The combined organic layers were dried over Na₂SO₄, filtered and concentrated under reduced pressure to give a residue. The residue was purified by column chromatography (SiO₂, petroleum ether:ethyl acetate=100:0 to 93:7) to give Intermediate Compound 17 (5.35 g, 9.99 mmol, 64.17% yield) as a light yellow oil.

### LCMS [M+1]⁺=536.4

¹H NMR (400 MHz, CHLOROFORM-d) δ=6.71-6.56 (m, 1H), 6.40 (dd, J=1.6, 16.8 Hz, 1H), 5.74 (br d, J=10.4 Hz, 1H), 5.44-5.24 (m, 2H), 4.31-4.16 (m, 4H), 3.73-3.64 (m, 4H), 2.37-2.27 (m, 3H), 2.10-1.98 (m, 4H), 1.67-1.54 (m, 4H), 1.49-1.34 (m, 4H), 1.25 (s, 20H), 0.96 (t, J=7.6 Hz, 3H), 0.88 (t, J=6.8 Hz, 6H)

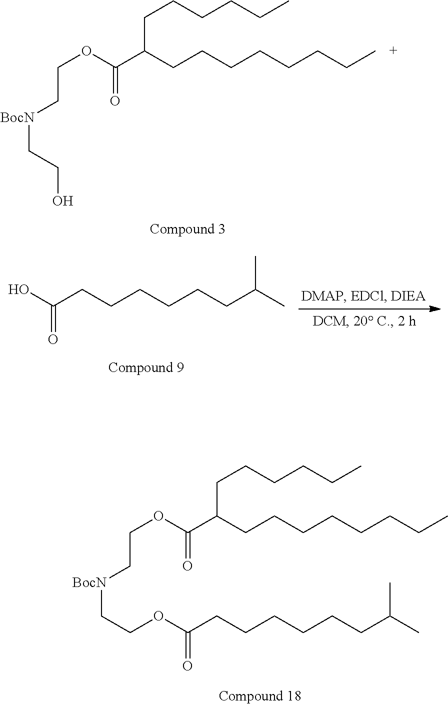 [SMILES: CCCCCCCCC(CCCCCC)C(=O)OCCN(CCO)C(=O)OC(C)(C)C ; CC(C)CCCCCCC(=O)O ; CCCCCCCCC(CCCCCC)C(=O)OCCN(CCOC(=O)CCCCCCC(C)C)C(=O)OC(C)(C)C]

To a solution of Compound 3 (8.5 g, 19.16 mmol, 1 eq.) and Compound 9 (3.96 g, 22.99 mmol, 1.2 eq.) in DCM (100 mL) was added DMAP (468.12 mg, 3.83 mmol, 0.2 eq.), EDCI (4.41 g, 22.99 mmol, 1.2 eq.) and DIEA (7.43 g, 57.48 mmol, 10.01 mL, 3 eq.). The mixture was stirred at 20° C. for 2 hours. TLC (petroleum ether:ethyl acetate=5:1, R_{f}=0.55) indicated Compound 3 was consumed completely and one new spot formed. The reaction mixture was concentrated under reduced pressure to give a residue. The residue was purified by column chromatography (SiO₂, petroleum ether:ethyl acetate=100:1 to 20:1) to give Compound 18 (10.02 g, 16.76 mmol, 87.47% yield) as a colorless oil.

LCMS [M−100+1]⁺=498.5

¹H NMR (400 MHz, CHLOROFORM-d) δ=4.18-4.02 (m, 4H), 3.54-3.28 (m, 4H), 2.32-2.15 (m, 3H), 1.59-1.40 (m, 7H), 1.39 (s, 9H), 1.29-1.13 (m, 26H), 1.08 (br d, J=6.8 Hz, 2H), 0.86-0.73 (m, 12H)

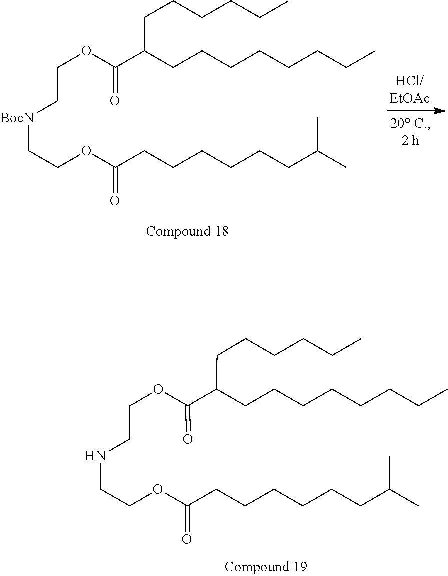 [SMILES: CCCCCCCCC(CCCCCC)C(=O)OCCN(CCOC(=O)CCCCCCC(C)C)C(=O)OC(C)(C)C ; CCCCCCCCC(CCCCCC)C(=O)OCCNCCOC(=O)CCCCCCC(C)C]

A solution of Compound 18 (8.5 g, 14.22 mmol, 1 eq.) in HCl/EtOAc (85 mL) was stirred at 20° C. for 2 hours. TLC (petroleum ether:ethyl acetate=5:1, R_{f}=0) indicated Compound 18 was consumed completely and one new spot formed. The reaction mixture was concentrated under reduced pressure to give Compound 19 (8 g, 14.08 mmol, 99.01% yield, 94% purity, HCl) as a colorless oil.

LCMS [M+1]⁺=498.5

¹H NMR (400 MHz, CHLOROFORM-d) δ=10.18-10.01 (m, 2H), 4.53 (br s, 4H), 3.45-3.27 (m, 4H), 2.51-2.25 (m, 4H), 1.71-1.40 (m, 8H), 1.26 (br s, 24H), 1.19-1.12 (m, 2H), 0.93-0.81 (m, 12H)

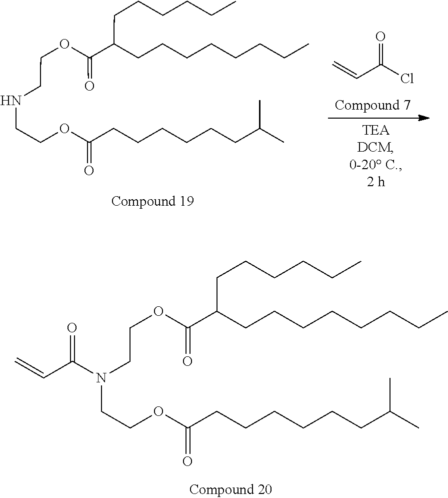

To a solution of Compound 19 (7.9 g, 15.87 mmol, 1 eq.) in DCM (80 mL) was added TEA (4.82 g, 47.61 mmol, 6.63 mL, 3 eq.) at 20° C., followed by Compound 7 (2.15 g, 23.81 mmol, 1.93 mL, 1.5 eq.) at 0° C. The mixture was stirred at 20° C. for 2 hours. TLC (petroleum ether:Ethyl acetate=3:1, R_{f}=0.45) indicated Compound 19 was consumed completely and one new spot formed. The reaction mixture was concentrated under reduced pressure to give a residue. The residue was purified by column chromatography (SiO₂, petroleum ether:ethyl acetate=20:1 to 5:1) to give Intermediate Compound 20 (7.15 g, 12.96 mmol, 81.64% yield) as a yellow oil.

LCMS [M+1]⁺=552.5

¹H NMR (400 MHz, CHLOROFORM-d) δ=6.71-6.58 (m, 1H), 6.40 (dd, J=2.0, 16.8 Hz, 1H), 5.74 (br d, J=10.4 Hz, 1H), 4.30-4.18 (m, 4H), 3.73-3.65 (m, 4H), 2.37-2.27 (m, 3H), 1.61 (br s, 2H), 1.58-1.37 (m, 5H), 1.33-1.21 (m, 26H), 1.15 (q, J=6.8 Hz, 2H), 0.93-0.82 (m, 12H)

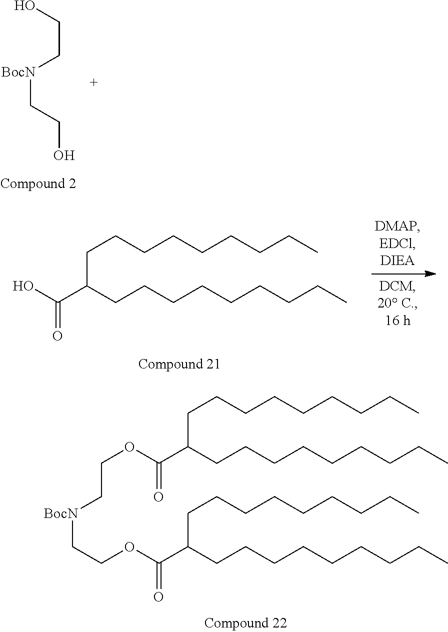 [SMILES: CC(C)(C)OC(=O)N(CCO)CCO ; CCCCCCCCCC(CCCCCCCCC)C(=O)O ; CCCCCCCCCC(CCCCCCCCC)C(=O)OCCN(CCOC(=O)C(CCCCCCCCC)CCCCCCCCC)C(=O)OC(C)(C)C]

To a solution of Compound 2 (3.35 g, 10.72 mmol, 2.2 eq.) and Compound 21 (1 g, 4.87 mmol, 921.66 μL, 1 eq.) in DCM (10 mL) was added EDCI (2.33 g, 12.18 mmol, 2.5 eq.), DIEA (1.89 g, 14.62 mmol, 2.55 mL, 3 eq.) and DMAP (119.04 mg, 974.42 μmol, 0.2 eq.). The mixture was stirred at 20° C. for 16 hours. TLC (petroleum ether:ethyl acetate=10:1, P1:R_{f}=0.48) indicated Compound 21 and Compound 2 was consumed completely and new spots formed. The reaction mixture was diluted with H₂O (10 mL) and extracted with DCM (10 mL×3). The combined organic layers were dried over Na₂SO₄, filtered and concentrated under reduced pressure to give a residue. The residue was purified by column chromatography (SiO₂, petroleum ether:ethyl acetate=100:1 to 0:1) to give Compound 22 (1.98 g, 2.49 mmol, 51.17% yield) as colorless oil.

LCMS [M−100+1]⁺=695.6.

¹H NMR (400 MHz, CHLOROFORM-d) δ=4.25-4.09 (m, 4H), 3.58-3.42 (m, 4H), 2.40-2.26 (m, 2H), 1.65-1.53 (m, 8H), 1.50-1.38 (m, 13H), 1.36-1.30 (m, 4H), 1.26 (s, 48H), 0.89 (t, J=6.8 Hz, 12H).

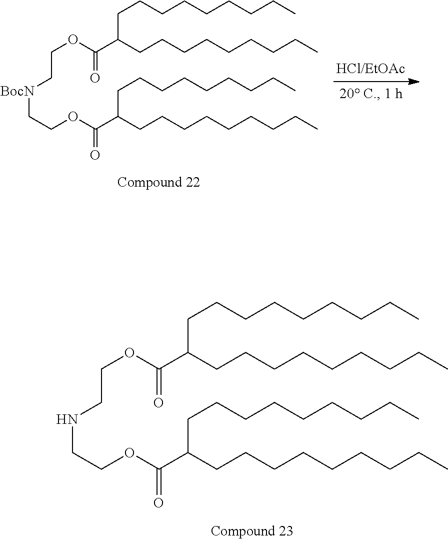 [SMILES: CCCCCCCCCC(CCCCCCCCC)C(=O)OCCN(CCOC(=O)C(CCCCCCCCC)CCCCCCCCC)C(=O)OC(C)(C)C ; CCCCCCCCCC(CCCCCCCCC)C(=O)OCCNCCOC(=O)C(CCCCCCCCC)CCCCCCCCC]

A solution of Compound 22 (1.98 g, 2.49 mmol, 1 eq) in HCl/EtOAc (4M, 15 mL) was stirred at 20° C. for 1 hour. TLC (petroleum ether:ethyl acetate=10:1, P1:R_{f}0.02) indicated Compound 22 was consumed completely and one new spot formed. The reaction mixture was concentrated to give Compound 23 (1.8 g, 2.46 mmol, 98% yield, HCl) as colorless oil.

LCMS [M+1]⁺=694.7.

¹H NMR (400 MHz, CHLOROFORM-d) δ=9.79 (br s, 2H), 4.50 (br s, 4H), 3.42-3.28 (m, 4H), 2.49-2.35 (m, 2H), 1.68-1.56 (m, 4H), 1.54-1.43 (m, 4H), 1.31 (br s, 4H), 1.26 (s, 48H), 0.89 (t, J=6.8 Hz, 12H).

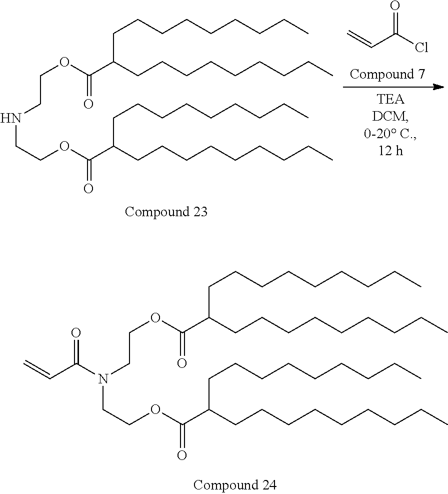

To a solution of Compound 4 (14 g, 20.17 mmol, 1 eq.) and TEA (6.12 g, 60.50 mmol, 8.42 mL, 3 eq.) in DCM (140 mL) was added Compound 5 (2.74 g, 30.25 mmol, 2.46 mL, 1.5 eq.) dropwise at 0° C. The resulting mixture was stirred at 20° C. for 16 hours. LCMS showed Compound 4 was consumed and desired MS was detected. The reaction mixture was poured into saturated NH₄C₁ (50 mL) and extracted with DCM (100 mL×2). The combined organic layers were washed with brine (50 mL), dried over Na₂SO₄, filtered and concentrated under reduced pressure to give a residue. The residue was purified by column chromatography (SiO₂, petroleum ether:ethyl acetate=55:1 to 17:1) to give Intermediate Compound 24 (10.9 g, 14.57 mmol, 72.23% yield, 100% purity) as white solid.

LCMS [M+1]⁺=748.7

¹H NMR (400 MHz, CHLOROFORM-d) δ=6.72-6.53 (m, 1H), 6.39 (dd, J=2.0, 16.8 Hz, 1H), 5.73 (dd, J=2.0, 10.4 Hz, 1H), 4.34-4.14 (m, 4H), 3.68 (t, J=6.0 Hz, 4H), 2.38-2.25 (m, 2H), 1.62-1.51 (m, 4H), 1.48-1.40 (m, 4H), 1.31-1.18 (m, 56H), 0.88 (t, J=6.8 Hz, 12H)

### General Procedure for Preparation of Series 900 Compounds 917a-i

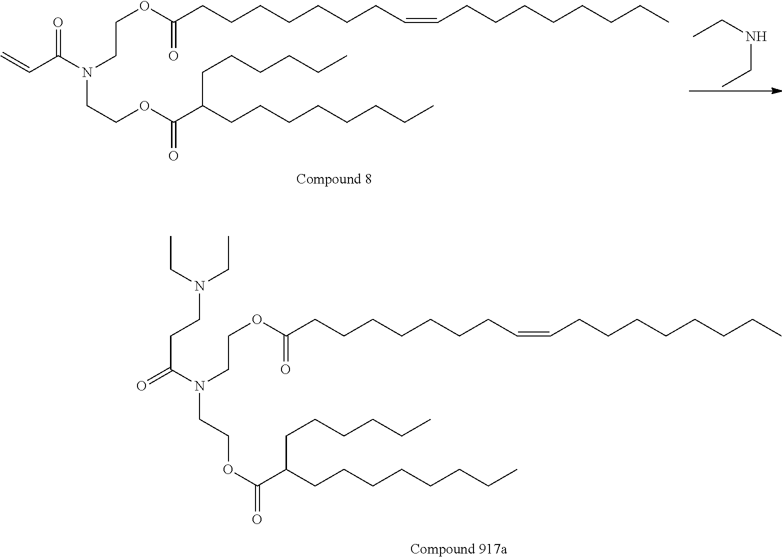

To a solution of Intermediate Compound 8 (100 mg, 0.151 mmol, 1 eq.) in ethanol (100 μL) was added diethylamine (0.151 mmol, 15.64 μL, 1 eq.) dissolved in 16 μL ethanol. The mixture was stirred at 20° C. for 18 hours.

The residue was purified by column chromatography (Biotage Selekt: silica gel 10 g column; mobile phase 100% acetone) to give Compound 917a (60.8 mg, 0.082 mmol, 54.71% yield, 94.15% purity) as a colorless oil.

Compound 917a: LCMS [M−100+1]⁺=735.19. ¹H NMR (400 MHz, chloroform-d) δ=5.39-5.29 (m, 2H), 4.24-4.13 (m, 4H), 3.62 (dtd, J=8.5, 5.9, 3.4 Hz, 4H), 2.81 (t, J=7.5 Hz, 2H), 2.54 (q, J=7.0 Hz, 6H), 2.30 (qd, J=6.6, 3.2 Hz, 4H), 2.01 (q, J=6.6 Hz, 4H), 1.68-1.50 (m, 5H), 1.44 (qd, J=7.5, 3.4 Hz, 2H), 1.38-1.20 (m, 40H), 1.04 (t, J=7.1 Hz, 6H), 0.92-0.85 (m, 9H).

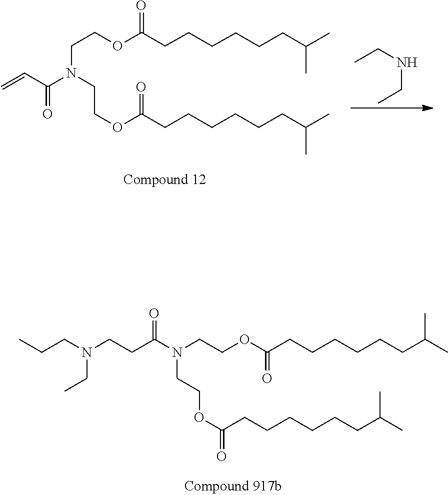

To a solution of Intermediate Compound 12 (100 mg, 0.213 mmol, 1 eq) in ethanol (100 μL) was added diethylamine (0.213 mmol, 22.14 μL, 1 eq.) dissolved in 22 μL ethanol. The mixture was stirred at 20° C. for 18 hours.

The residue was purified by column chromatography (Biotage Selekt: silica gel 10 g column; mobile phase 100% acetone) to give Compound 917b (69.1 mg, 0.128 mmol, 59.7% yield, 61.87% purity) as a colorless oil.

Compound 917b: LCMS [M−100+1]⁺=541.5. ¹H NMR (400 MHz, chloroform-d) δ=4.20 (q, J=5.8 Hz, 4H), 3.61 (dt, J=8.5, 5.8 Hz, 4H), 2.80 (dd, J=8.6, 6.6 Hz, 2H), 2.58-2.47 (m, 6H), 2.30 (td, J=7.6, 4.1 Hz, 4H), 1.60 (p, J=7.5 Hz, 4H), 1.50 (dq, J=13.2, 6.6 Hz, 2H), 1.36-1.22 (m, 12H), 1.21-1.09 (m, 4H), 1.03 (t, J=7.1 Hz, 6H), 0.86 (d, J=6.6 Hz, 12H).

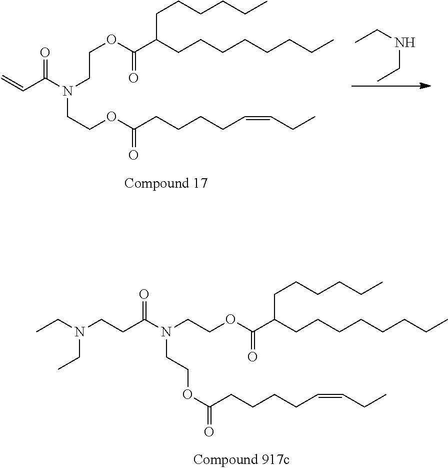

To a solution of Intermediate Compound 17 (100 mg, 0.186 mmol, 1 eq.) in ethanol (100 μL) was added diethylamine (0.186 mmol, 19.32 μL, 1 eq.) dissolved in 19 μL ethanol. The mixture was stirred at 20° C. for 18 hours.

The residue was purified by column chromatography (Biotage Selekt: silica gel 10 g column; mobile phase 100% acetone) to give Compound 917c (66.1 mg, 0.109 mmol, 58.1% yield, 85.52% purity) as a colorless oil.

Compound 917c: LCMS [M−100+1]⁺=609.7. ¹H NMR (400 MHz, chloroform-d) δ=5.43-5.23 (m, 2H), 4.24-4.15 (m, 4H), 3.62 (dtd, J=8.4, 5.9, 3.5 Hz, 4H), 2.80 (dd, J=8.6, 6.5 Hz, 2H), 2.53 (p, J=6.4 Hz, 6H), 2.31 (td, J=7.7, 3.6 Hz, 3H), 2.09-1.97 (m, 4H), 1.62 (s, 4H), 1.49-1.25 (m, 4H), 1.25 (s, 20H), 1.03 (t, J=7.1 Hz, 6H), 0.95 (t, J=7.5 Hz, 3H), 0.87 (t, J=6.7 Hz, 6H).

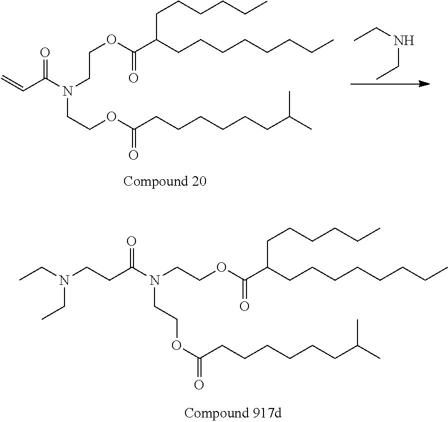

To a solution of Intermediate Compound 20 (100 mg, 0.181 mmol, 1 eq.) in ethanol (100 μL) was added diethylamine (0.181 mmol, 18.76 μL, 1 eq.) dissolved in 19 μL ethanol. The mixture was stirred at 20° C. for 18 hours. The residue was purified by column chromatography (Biotage Selekt: silica gel 10 g column; mobile phase 100% acetone) to give Compound 917d (76.9 mg, 0.123 mmol, 67.85% yield, 76.21% purity) as a colorless oil.

Compound 917d: LCMS [M−100+1]⁺=625.6. ¹H NMR (400 MHz, chloroform-d) δ=4.24-4.15 (m, 4H), 3.62 (dtd, J=8.6, 5.9, 3.5 Hz, 4H), 2.80 (dd, J=8.6, 6.5 Hz, 2H), 2.53 (q, J=7.1 Hz, 6H), 2.37-2.25 (m, 3H), 1.66-1.39 (m, 8H), 1.36-1.18 (m, 26H), 1.03 (t, J=7.1 Hz, 6H), 0.91-0.83 (m, 12H).

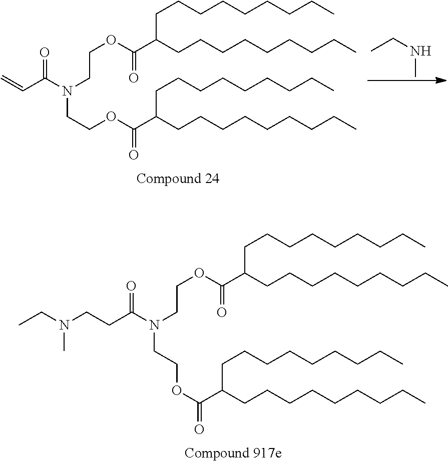

To a solution of Intermediate Compound 24 (100 mg, 0.134 mmol, 1 eq.) in ethanol (100 μL) was added ethylmethylamine (0.134 mmol, 11.49 μL, 1 eq.) dissolved in 12 μL ethanol. The mixture was stirred at 45° C. for 18 hours. The residue was purified by column chromatography (Biotage Selekt: silica gel 10g column; mobile phase 100% acetone) to give Compound 917e (56.4 mg, 0.0698 mmol, 52.23% yield, 53.11% purity) as a colorless oil.

Compound 917e: LCMS [M−100+1]⁺=807.8. ¹H NMR (400 MHz, chloroform-d) δ=4.20 (q, J=5.8 Hz, 4H), 3.67-3.57 (m, 4H), 2.73 (t, J=7.5 Hz, 2H), 2.56 (t, J=7.6 Hz, 2H), 2.45 (q, J=7.2 Hz, 2H), 2.31 (ttd, J=8.6, 5.7, 2.8 Hz, 2H), 2.25 (s, 3H), 1.64-1.52 (m, 6H), 1.48-1.39 (m, 4H), 1.35-1.15 (m, 54H), 1.07 (t, J=7.2 Hz, 3H), 0.92-0.83 (m, 12H).

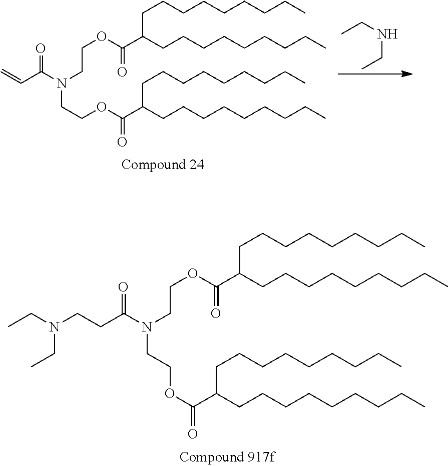

To a solution of Intermediate Compound 24 (100 mg, 0.134 mmol, 1 eq.) in ethanol (100 μL) was added diethylamine (0.134 mmol, 13.84 μL, 1 eq.) dissolved in 14 μL ethanol. The mixture was stirred at 20° C. for 18 hours. The residue was purified by column chromatography (Biotage Selekt: silica gel 10 g column; mobile phase 100% acetone) to give Compound 917f (57.7 mg, 0.071 mmol, 52.5% yield, 91.31% purity) as a colorless oil.

Compound 917f: LCMS [M−100+1]⁺=821.7. ¹H NMR (400 MHz, chloroform-d) δ=4.20 (q, J=5.8 Hz, 4H), 3.62 (dt, J=11.4, 6.0 Hz, 4H), 2.81 (t, J=7.5 Hz, 2H), 2.54 (q, J=7.1 Hz, 6H), 2.31 (ttd, J=8.2, 5.5, 2.0 Hz, 2H), 1.64-1.50 (m, 6H), 1.44 (tt, J=13.9, 5.0 Hz, 4H), 1.35-1.13 (m, 54H), 1.03 (t, J=7.1 Hz, 6H), 0.96-0.80 (m, 12H).

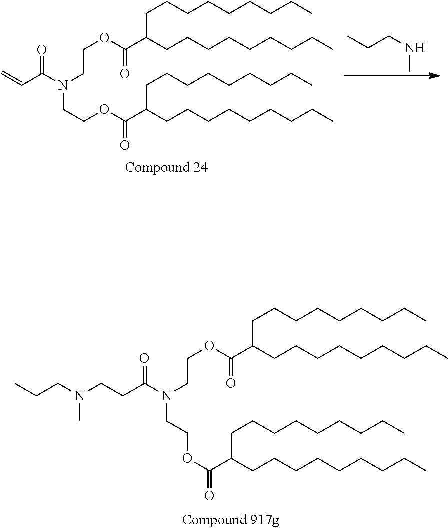

To a solution of Intermediate Compound 24 (100 mg, 0.134 mmol, 1 eq.) in ethanol (100 μL) was added methylpropylamine (0.134 mmol, 13.72 μL, 1 eq.) dissolved in 14 μL ethanol. The mixture was stirred at 20° C. for 18 hours. The residue was purified by column chromatography (Biotage Selekt: silica gel 10 g column; mobile phase 100% acetone) to give Compound 917g (70.1 mg, 0.0853 mmol, 63.8% yield, 93.39% purity) as a colorless oil.

Compound 917g: LCMS [M−100+1]⁺=821.8. ¹H NMR (400 MHz, chloroform-d) δ=4.20 (td, J=5.9, 3.8 Hz, 4H), 3.62 (dt, J=8.6, 6.0 Hz, 4H), 2.72 (dd, J=8.5, 6.5 Hz, 2H), 2.55 (dd, J=8.5, 6.5 Hz, 2H), 2.38-2.26 (m, 4H), 2.24 (s, 3H), 1.67-1.36 (m, 12H), 1.34-1.14 (m, 54H), 0.88 (td, J=7.2, 3.4 Hz, 15H).

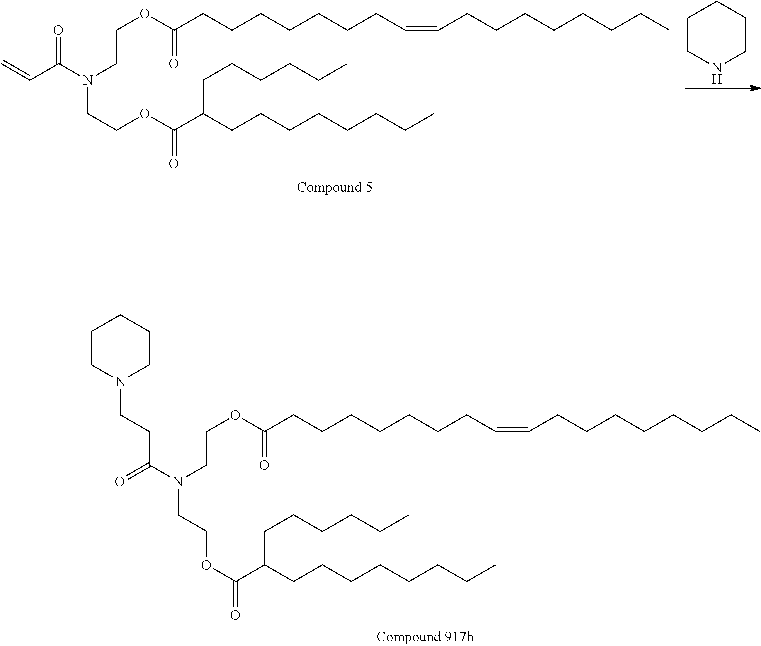

To a solution of Intermediate Compound 8 (100 mg, 0.151 mmol, 1 eq.) in ethanol (100 μL) was added piperidine (0.151 mmol, 14.94 μL, 1 eq.) dissolved in 15 μL ethanol. The mixture was stirred at 20° C. for 18 hours. The residue was purified by column chromatography (Biotage Selekt: silica gel 10 g column; mobile phase 100% acetone) to give Compound 917h (89.9 mg, 0.1203 mmol, 79.59% yield, 84.82% purity) as a colorless oil.

Compound 917h: LCMS [M−100+1]⁺=747.8. ¹H NMR (400 MHz, chloroform-d) δ=5.39-5.28 (m, 2H), 4.24-4.15 (m, 4H), 3.67-3.56 (m, 4H), 2.68 (ddt, J=8.4, 5.5, 1.5 Hz, 2H), 2.57 (dt, J=9.0, 5.7 Hz, 2H), 2.41 (s, 3H), 2.36-2.25 (m, 3H), 2.01 (q, J=6.5 Hz, 4H), 1.59 (s, 10H), 1.45 (t, J=5.7 Hz, 4H), 1.35-1.20 (m, 42H), 0.92-0.84 (m, 9H).

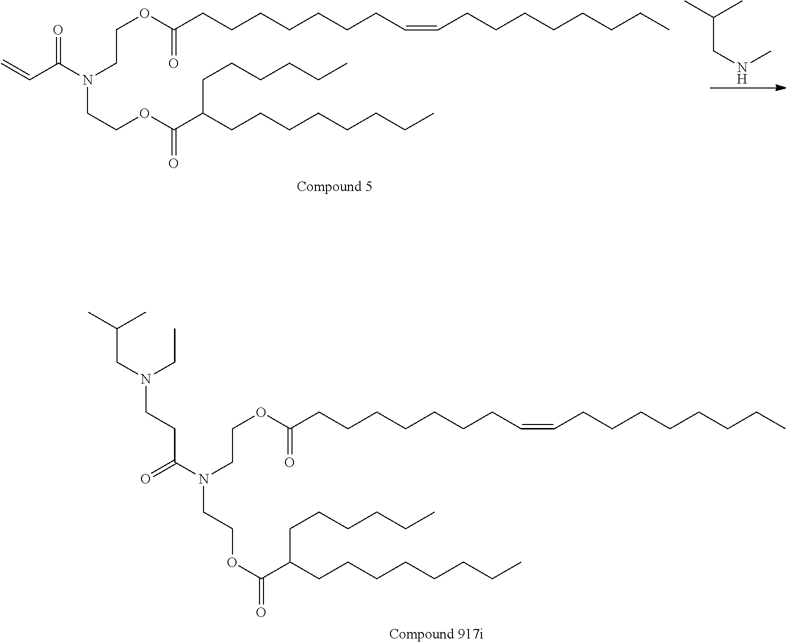

To a solution of Intermediate Compound 8 (100 mg, 0.151 mmol, 1 eq.) in ethanol (100 μL) was added methylisopropylamine (0.151 mmol, 18.05 μL, 1 eq.) dissolved in 18 μL ethanol. The mixture was stirred at 20° C. for 18 hours. The residue was purified by column chromatography (Biotage Selekt: silica gel 10 g column; mobile phase 100% acetone) to give Compound 917i (66.3 mg, 0.0885 mmol, 58.54% yield, 89.53% purity) as a colorless oil.

Compound 917i: LCMS [M−100+1]⁺=747.7. ¹H NMR (400 MHz, chloroform-d) δ=5.39-5.28 (m, 2H), 4.20 (td, J=6.0, 3.0 Hz, 4H), 3.60 (td, J=5.7, 2.0 Hz, 4H), 2.69 (t, J=7.3 Hz, 2H), 2.53 (q, J=7.3 Hz, 2H), 2.31 (qd, J=8.2, 5.1 Hz, 3H), 2.21 (d, J=1.7 Hz, 3H), 2.10 (d, J=7.2 Hz, 2H), 2.00 (p, J=5.7 Hz, 4H), 1.66-1.50 (m, 5H), 1.43 (dt, J=13.8, 4.5 Hz, 2H), 1.37-1.20 (m, 40H), 0.95-0.84 (m, 15H).

### General Procedure for Purification of Compound 918a:

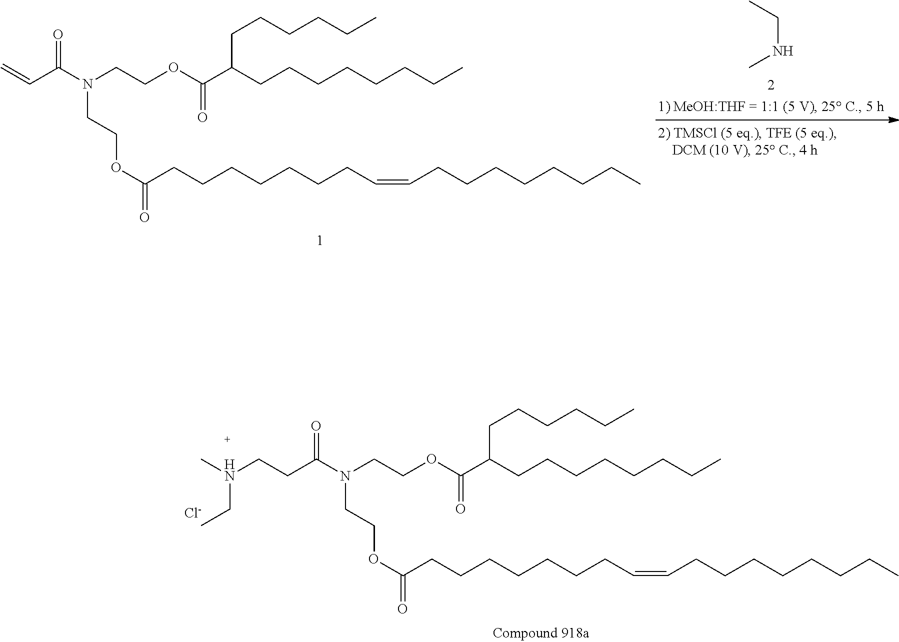

To a solution of Compound 1 (1.00 g, 1.51 mmol, 1 eq.) in THF (5 mL) and MeOH (5 mL) was added Compound 2 (178.57 mg, 3.02 mmol, 259.55 μL, 2 eq.). The mixture was stirred at 25° C. for 5 hours. TLC (Petroleum ether:Ethyl acetate=5:1, R_{f}=0.42) indicated Compound 1 was consumed completely and one new spot formed. The reaction mixture was concentrated under reduced pressure to give a residue. The residue was purified by column chromatography (SiO₂, Petroleum ether:Ethyl acetate=100:1 to 60:40) to give the crude product. The crude product was re-purified by prep-HPLC (column: X-Select CSH Phenyl-Hexyl 100×30 5u; mobile phase: [H₂O (0.04% HCl)-ACN:THF=1:1]; gradient: 40%-85% B over 16.0 min) and (SiO₂, Petroleum ether:Ethyl acetate=100:1 to 60:40) to give Compound 918a (357 mg, 492.57 μmol, 32.61% yield, 99.50% purity, Free) as a light-yellow oil.

LCMS [M+1]*=721.7

¹H NMR (400 MHz, CHLOROFORM-d) δ=5.43-5.24 (m, 2H), 4.28-4.17 (m, 4H), 3.74-3.57 (m, 4H), 3.40-3.18 (m, 2H), 3.17-2.86 (m, 4H), 2.68 (br s, 3H), 2.32 (td, J=7.6, 10.8 Hz, 3H), 2.05-1.95 (m, 4H), 1.60-1.52 (m, 4H), 1.47-1.38 (m, 5H), 1.36-1.17 (m, 40H), 0.88 (t, J=6.8 Hz, 9H).

A mixture of TFE (246.93 mg, 2.47 mmol, 177.52 μL, 5 eq.) and TMSCI (268.16 mg, 2.47 mmol, 313.27 μL, 5 eq.) was stirred at 25° C. for 1 hour. Then, Compound 918a (356 mg, 493.66 μmol, 1 eq.) in DCM (3 mL) was added. The resulting mixture was stirred at 25° C. for 3 hours. The reaction mixture was concentrated under reduced pressure and lyophilized to give a residue. The residue was purified by column chromatography (SiO₂, DCM:MeOH=100:0 to 100:1) to give Compound 918a (300 mg, 389.21 μmol, 78.84% yield, 98.29% purity, 1.0 HCl) as colorless gum.

LCMS [M+1]⁺=721.7

¹H NMR (400 MHz, CHLOROFORM-d) δ=12.62-12.23 (m, 1H), 5.44-5.24 (m, 2H), 4.31-4.13 (m, 4H), 3.74-3.65 (m, 2H), 3.61 (q, J=5.6 Hz, 2H), 3.51-3.42 (m, 1H), 3.37-3.29 (m, 1H), 3.28-3.13 (m, 3H), 3.05 (td, J=6.4, 12.8 Hz, 1H), 2.75 (d, J=4.8 Hz, 3H), 2.33 (td, J=7.6, 12.0 Hz, 3H), 2.01 (br d, J=5.6 Hz, 4H), 1.61-1.52 (m, 4H), 1.47 (br t, J=7.2 Hz, 5H), 1.37-1.18 (m, 40H), 0.88 (t, J=6.8 Hz, 9H)

### General Procedure for Preparation of Compound 918b

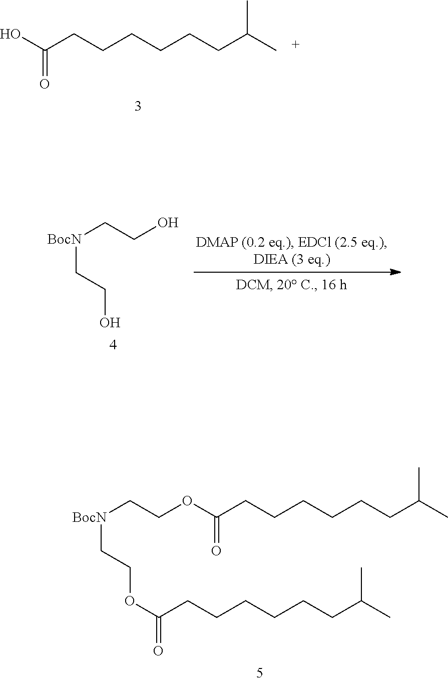 [SMILES: CC(C)CCCCCCC(=O)O ; CC(C)(C)OC(=O)N(CCO)CCO ; CC(C)CCCCCCC(=O)OCCN(CCOC(=O)CCCCCCC(C)C)C(=O)OC(C)(C)C]

To a solution of Compound 3 (9.79 g, 56.81 mmol, 2.2 eq.) in DCM (53 mL) was added DIEA (10.01 g, 77.47 mmol, 13.49 mL, 3 eq.), EDCI (12.38 g, 64.56 mmol, 2.5 eq.), DMAP (630.94 mg, 5.16 mmol, 0.2 eq.) and Compound 4 (5.3 g, 25.82 mmol, 4.88 mL, 1 eq.). The mixture was stirred at 25° C. for 16 hours. LCMS showed Compound 4 was consumed completely and one main peak with desired MS was detected. The reaction mixture was diluted with H₂O (90 mL) and extracted with DCM (30 mL×3); the combined organic layers were dried over Na₂SO₄, filtered and concentrated under reduced pressure to give a residue. The residue was purified by column chromatography (SiO₂, Petroleum ether:Ethyl acetate=99:1 to 95:5) to give Compound 5 (5.82 g, 10.94 mmol, 42.38% yield, 96.6% purity) as a colorless oil.

LCMS [M−100+1]⁺=414.3

¹H NMR (400 MHz, CHLOROFORM-d) δ=4.25-4.12 (m, 4H), 3.58-3.41 (m, 4H), 2.31 (t, J=7.6 Hz, 4H), 1.67-1.59 (m, 4H), 1.56-1.49 (m, 2H), 1.47 (s, 9H), 1.36-1.23 (m, 12H), 1.15 (q, J=6.8 Hz, 4H), 0.87 (d, J=6.8 Hz, 12H)

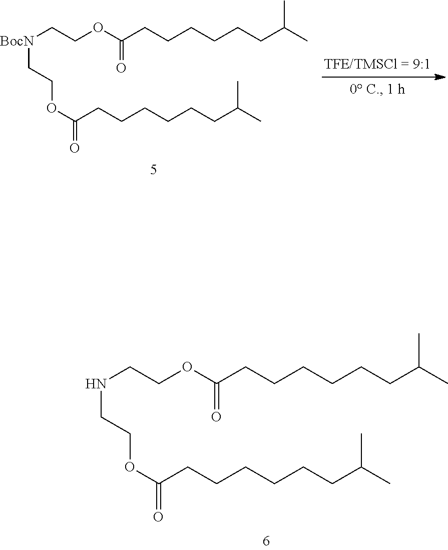 [SMILES: CC(C)CCCCCCC(=O)OCCN(CCOC(=O)CCCCCCC(C)C)C(=O)OC(C)(C)C ; CC(C)CCCCCCC(=O)OCCNCCOC(=O)CCCCCCC(C)C]

A solution of Compound 5 (8.12 g, 15.81 mmol, 1 eq.) in TFE (72 mL) and TMSCI (8 mL) was stirred at 0° C. for 1 hour. LCMS showed Compound 5 was consumed completely, and one main peak with desired MS was detected. The reaction mixture was concentrated under reduced pressure to give Compound 6 (HCl salt). The Compound 6 (HCl salt) was dissolved in DCM (10 mL) and then basified with saturated NaHCO₃ to pH=7. The organic layer was separated and washed with brine (10 mL), dried over Na₂SO₄, filtered and concentrated under reduced pressure to give Compound 6 (6.5 g, 14.88 mmol, 94.17% yield, 94.72% purity) as a colorless oil.

LCMS [M+1]⁺=414.4

¹H NMR (400 MHz, CHLOROFORM-d) δ=4.19 (t, J=5.6 Hz, 4H), 2.91 (t, J=5.6 Hz, 4H), 2.33 (t, J=7.6 Hz, 4H), 1.65-1.58 (m, 4H), 1.52 (quind, J=6.8, 13.2 Hz, 2H), 1.37-1.22 (m, 12H), 1.21-1.10 (m, 4H), 0.86 (d, J=6.8 Hz, 12H)

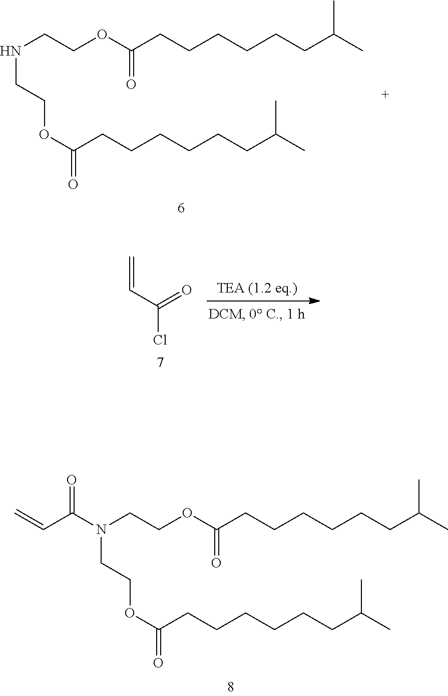

To a solution of Compound 6 (6.5 g, 15.71 mmol, 1 eq.) in DCM (65 mL) was added TEA (1.91 g, 18.86 mmol, 2.62 mL, 1.2 eq.) and Compound 7 (1.71 g, 18.86 mmol, 1.53 mL, 1.2 eq.). The mixture was stirred at 0° C. for 1 hour. LCMS showed Compound 6 was consumed completely and one main peak with desired MS was detected. The reaction mixture was diluted with H₂O (20 mL) and extracted with (5 mL×3); the organic layers were dried over Na₂SO₄, filtered and concentrated under reduced pressure to give a residue. The residue was purified by column chromatography (SiO₂, Petroleum ether:Ethyl acetate=99:1 to 96:4) to give Compound 8 (5.42 g, 11.14 mmol, 70.89% yield, 96.13% purity) as a colorless oil.

LCMS [M+1]⁺=468.4

¹H NMR (400 MHz, CHLOROFORM-d) δ=6.63 (dd, J=10.4, 16.8 Hz, 1H), 6.39 (dd, J=2.0, 16.8 Hz, 1H), 5.74 (dd, J=2.0, 10.4 Hz, 1H), 4.32-4.17 (m, 4H), 3.73-3.64 (m, 4H), 2.30 (dt, J=4.0, 7.6 Hz, 4H), 1.67-1.58 (m, 4H), 1.56-1.46 (m, 2H), 1.28 (br s, 12H), 1.16 (q, J=6.8 Hz, 4H), 0.87 (d, J=6.8 Hz, 12H)

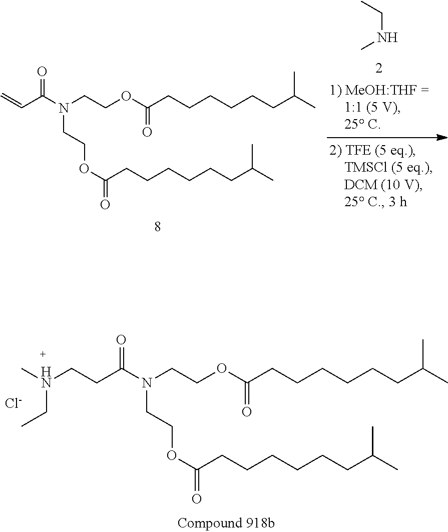

To a solution of Compound 8 (600 mg, 1.28 mmol, 1 eq.) in THF (1.5 mL) and MeOH (1.5 mL) was added Compound 2 (227.50 mg, 3.85 mmol, 330.67 μL, 3 eq.). The mixture was stirred at 20° C. for 3 hours. LCMS showed Compound 8 was consumed completely and one main peak with desired MS was detected. The reaction mixture was concentrated to give a residue. The residue was purified by column chromatography (SiO₂, Petroleum ether:Ethyl acetate=60:40 to 0:100) to give Compound 918b (174 mg, 327.92 μmol, 25.56% yield, 99.28% purity, free) as a colorless oil.

LCMS [M+1]⁺=527.5

¹H NMR (400 MHz, CHLOROFORM-d) δ=4.21 (q, J=5.6 Hz, 4H), 3.62 (q, J=5.6 Hz, 4H), 2.79-2.70 (m, 2H), 2.61-2.53 (m, 2H), 2.48 (q, J=7.2 Hz, 2H), 2.31 (dt, J=5.2, 7.6 Hz, 4H), 2.27 (s, 3H), 1.64-1.57 (m, 4H), 1.56-1.46 (m, 2H), 1.35-1.23 (m, 12H), 1.21-1.12 (m, 4H), 1.08 (t, J=7.2 Hz, 3H), 0.87 (d, J=6.8 Hz, 12H)

A solution of TMSCI (169.11 mg, 1.56 mmol, 197.56 μL, 5 eq.) in TFE (155.72 mg, 1.56 mmol, 111.95 μL, 5 eq.) was stirred at 20° C. for 1 hour. Then, Compound 918b (164 mg, 311.32 μmol, 1 eq., free) in DCM (1.64 mL) was added at 20° C. The resulting mixture was stirred at 20° C. for 3 hours. The reaction mixture was concentrated to give Compound 918b (140 mg, 248.51 μmol, 79.83% yield, 98.04% purity, 0.7 HCl) as a colorless gum.

LCMS [M+1]⁺=527.5

¹H NMR (400 MHz, CHLOROFORM-d) δ=12.41 (br s, 1H), 4.29-4.15 (m, 4H), 3.75-3.56 (m, 4H), 3.54-3.41 (m, 1H), 3.39-3.28 (m, 1H), 3.19 (br s, 3H), 3.11-2.98 (m, 1H), 2.75 (d, J=4.4 Hz, 3H), 2.32 (td, J=7.6, 11.2 Hz, 4H), 1.62-1.43 (m, 9H), 1.28 (br s, 12H), 1.19-1.10 (m, 4H), 0.86 (d, J=6.8 Hz, 12H)

### General Procedure for Preparation of Compound 918c

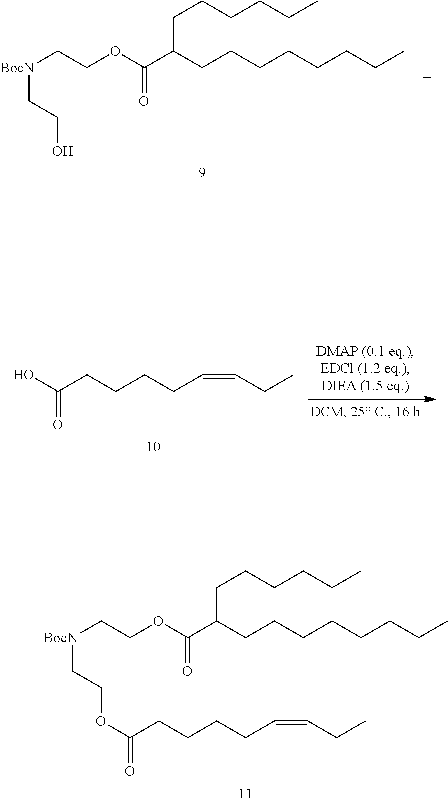 [SMILES: CCCCCCCCC(CCCCCC)C(=O)OCCN(CCO)C(=O)OC(C)(C)C ; CC/C=C\CCCCC(=O)O ; CC/C=C\CCCCC(=O)OCCN(CCOC(=O)C(CCCCCC)CCCCCCCC)C(=O)OC(C)(C)C]

To a mixture of Compound 10 (3.1 g, 19.84 mmol, 1 eq.) in DCM (88 mL) was added DMAP (242.42 mg, 1.98 mmol, 0.1 eq.) and EDCI (4.56 g, 23.81 mmol, 1.2 eq.), DIEA (3.85 g, 29.77 mmol, 5.18 mL, 1.5 eq.) and Compound 9 (8.80 g, 19.84 mmol, 1 eq.) at 25° C. The mixture was stirred at 25° C. for 16 hours. TLC (Petroleum ether:Ethyl acetate=8:1, R_{f}=0.53) indicated Compound 9 was consumed completely and one major new spot was formed. The reaction mixture was diluted with H₂O (150 mL) and extracted with DCM (200 mL). The combined organic layer was dried over Na₂SO₄, filtered and concentrated under reduced pressure to give a residue. The residue was purified by flash silica gel chromatography (ISCO®; 330 g SepaFlash® Silica Flash Column, 0-50% Ethyl acetate: Petroleum ether gradient @100 mL/min) to give (8.87 g, 15.05 mmol, 75.82% yield, 98.70% purity) as a colorless oil.

LCMS [M−100+1]⁺=482.4

¹H NMR (400 MHz, CHLOROFORM-d) δ=5.46-5.22 (m, 2H), 4.27-4.06 (m, 4H), 3.63-3.42 (m, 4H), 2.32 (br t, J=7.2 Hz, 3H), 2.13-1.99 (m, 4H), 1.70-1.58 (m, 4H), 1.47 (s, 9H), 1.45-1.33 (m, 4H), 1.26 (s, 20H), 0.96 (t, J=7.6 Hz, 3H), 0.88 (t, J=6.8 Hz, 6H)

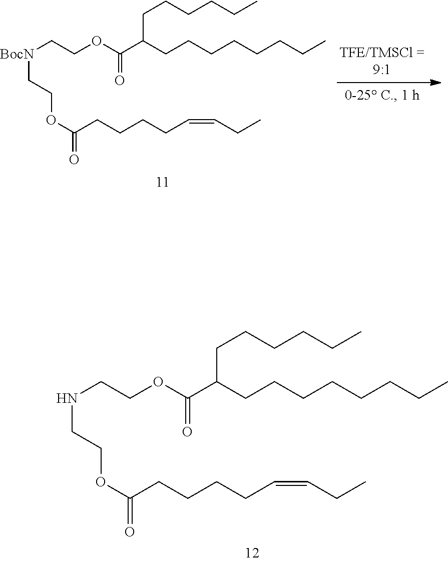 [SMILES: CC/C=C\CCCCC(=O)OCCN(CCOC(=O)C(CCCCCC)CCCCCCCC)C(=O)OC(C)(C)C ; CC/C=C\CCCCC(=O)OCCNCCOC(=O)C(CCCCCC)CCCCCCCC]

A solution of Compound 11 (8.87 g, 15.24 mmol, 1 eq.) in TFE (81 mL) and TMSCI (9 mL) was stirred at 0° C. for 1 hour. TLC (Petroleum ether:Ethyl acetate=5:1, R_{f}=0.1) indicated Compound 11 was consumed completely and one new spot formed. The reaction mixture was concentrated under reduced pressure to give Compound 12 (HCl salt). Compound 12 (HCl salt) was dissolved in DCM (15 mL) and basified with saturated NaHCO₃ (30 mL) to pH 7-8 at 0° C. The organic phase was separated and the aqueous phase extracted with DCM (15 mL×2). The combined organic layers were dried over Na₂SO₄, filtered and concentrated under reduced pressure to give Compound 12 (6.3 g, 13.08 mmol, 97.93% purity, Free) as a yellow oil.

LCMS [M+1]⁺=482.4

¹H NMR (400 MHz, CHLOROFORM-d) δ=5.48-5.23 (m, 2H), 4.19 (q, J=5.6 Hz, 4H), 2.90 (t, J=5.6 Hz, 4H), 2.43-2.27 (m, 3H), 2.12-1.95 (m, 4H), 1.71-1.60 (m, 4H), 1.49-1.36 (m, 4H), 1.31-1.21 (m, 20H), 0.96 (t, J=7.6 Hz, 3H), 0.88 (t, J=6.8 Hz, 6H)

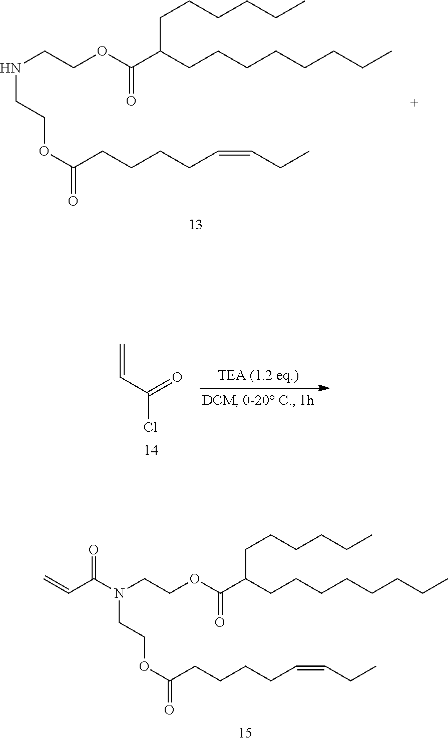

To a solution of Compound 13 (6.3 g, 13.08 mmol 1 eq.) and TEA (1.59 g, 15.69 mmol, 2.18 mL, 1.2 eq.) in DCM (63 mL) was added Compound 14 (1.42 g, 15.69 mmol, 1.27 mL, 1.2 eq.) at 0° C. The reaction mixture was stirred at 25° C. for 1 hour. TLC (Petroleum ether:Ethyl acetate=5:1, R_{f}=0.45) indicated Compound 13 was consumed completely. The reaction mixture was diluted with H₂O (100 mL) and extracted with DCM (80 mL×2). The combined organic layers were dried over Na₂SO₄, filtered and concentrated under reduced pressure to give a residue. The residue was purified by column chromatography (SiO₂, Petroleum ether:Ethyl acetate=50:1 to 3:1) to give Compound 15 (6 g, 11.20 mmol, 96.31% purity) as a light yellow oil.

LCMS [M+1]⁺=536.4

¹H NMR (400 MHz, CHLOROFORM-d) δ=6.72-6.53 (m, 1H), 6.40 (dd, J=2.0, 16.8 Hz, 1H), 5.74 (br d, J=10.0 Hz, 1H), 5.46-5.22 (m, 2H), 4.33-4.13 (m, 4H), 3.75-3.64 (m, 4H), 2.31 (br d, J=6.4 Hz, 3H), 2.13-1.93 (m, 4H), 1.69-1.57 (m, 4H), 1.48-1.34 (m, 4H), 1.26 (s, 20H), 0.96 (t, J=7.6 Hz, 3H), 0.92-0.83 (m, 6H)

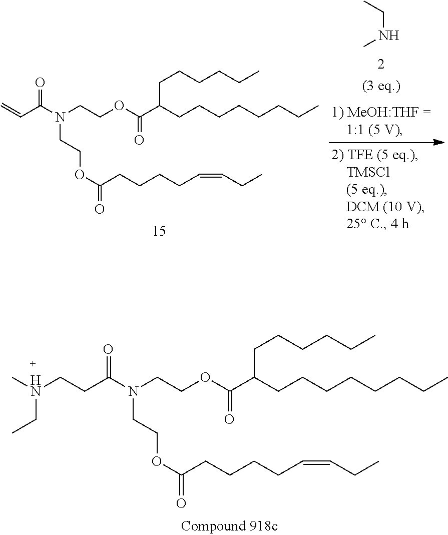

To a solution of Compound 15 (688 mg, 1.28 mmol, 1 eq.) in THF (1.7 mL) and MeOH (1.7 mL) was added Compound 2 (227.70 mg, 3.85 mmol, 330.96 μL, 3 eq.) at 20° C. The mixture was stirred at 20° C. for 2 hours. TLC (Petroleum ether:Ethyl acetate=3:1, R_{f}=0.00) indicated Compound 15 was consumed completely and one major new spot was formed. The reaction mixture was concentrated under reduced pressure to give a residue. The residue was purified by flash silica gel chromatography (ISCO®; 20 g SepaFlash® Silica Flash Column, 0-50% Ethyl acetate:Petroleum ether gradient @100 mL/min) to give Compound 918c (180 mg, 297.52 μmol, 23.17% yield, 98.33% purity, Free) as a light yellow oil.

LCMS [M+1]⁺=595.5

¹H NMR (400 MHz, CHLOROFORM-d) δ=5.51-5.21 (m, 2H), 4.29-4.15 (m, 4H), 3.71-3.52 (m, 4H), 2.87-2.44 (m, 6H), 2.36-2.24 (m, 6H), 2.10-1.95 (m, 4H), 1.68-1.60 (m, 4H), 1.46-1.33 (m, 4H), 1.26 (s, 20H), 1.10 (br t, J=7.2 Hz, 3H), 0.96 (t, J=7.6 Hz, 3H), 0.88 (t, J=6.8 Hz, 6H)

A mixture of TMSCI (164.36 mg, 1.51 mmol, 192.00 μL, 5 eq.) in TFE (151.34 mg, 1.51 mmol, 108.80 μL, 5 eq.) was stirred at at 25° C. for 1 hour, and then Compound 918c (180 mg, 302.57 μmol, 1 eq.) in DCM (1.8 mL) was added dropwise at 25° C. The resulting mixture was stirred at 25° C. for 3 hours. The reaction mixture was concentrated under reduced pressure to give Compound 918c (180 mg, 284.97 μmol, 94.18% yield, 99.38% purity, 0.9 HCl) as a light yellow gum.

LCMS [M+1]⁺=595.5

¹H NMR (400 MHz, CHLOROFORM-d) δ=12.59-12.26 (m, 1H), 5.45-5.25 (m, 2H), 4.29-4.15 (m, 4H), 3.68 (br t, J=5.6 Hz, 2H), 3.61 (q, J=5.6 Hz, 2H), 3.55-3.38 (m, 1H), 3.38-3.26 (m, 1H), 3.26-3.10 (m, 3H), 3.08-2.98 (m, 1H), 2.75 (d, J=4.8 Hz, 3H), 2.34 (td, J=7.6, 13.2 Hz, 3H), 2.11-1.95 (m, 4H), 1.69-1.61 (m, 2H), 1.59 (br s, 2H), 1.47 (t, J=7.2 Hz, 4H), 1.44-1.34 (m, 3H), 1.32-1.20 (m, 20H), 0.96 (t, J=7.6 Hz, 3H), 0.92-0.84 (m, 6H).

### General Procedure for Preparation of Compound 918d

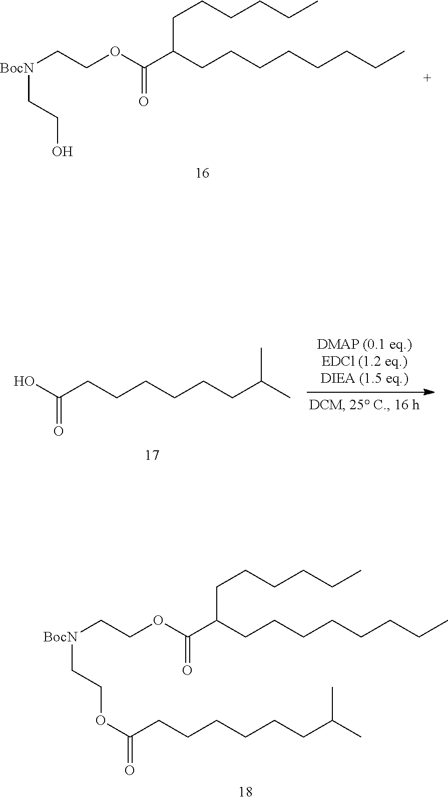 [SMILES: CCCCCCCCC(CCCCCC)C(=O)OCCN(CCO)C(=O)OC(C)(C)C ; CC(C)CCCCCCC(=O)O ; CCCCCCCCC(CCCCCC)C(=O)OCCN(CCOC(=O)CCCCCCC(C)C)C(=O)OC(C)(C)C]

To a solution of Compound 17 (3.11 g, 18.03 mmol, 1 eq.) in DCM (80 mL) was added DMAP (220.29 mg, 1.80 mmol, 0.1 eq.), EDCI (4.15 g, 21.64 mmol, 1.2 eq.), DIEA (3.50 g, 27.05 mmol, 4.71 mL, 1.5 eq.) and Compound 16 (8 g, 18.03 mmol, 1 eq.). The mixture was stirred at 25° C. for 16 hours. TLC (Petroleum ether:Ethyl acetate=5:1, R_{f}=0.82) indicated Compound 17 was consumed and new spots formed. LCMS showed Compound 16 remained and desired MS was detected. The reaction mixture was diluted with H₂O (80 mL) and extracted with DCM (80 mL×3). The combined organic layers were washed with brine (50 mL), dried over Na₂SO₄, filtered and concentrated under reduced pressure to give a residue (13.65 g). The residue was purified by flash silica gel chromatography (ISCO®; 120 g SepaFlash® Silica Flash Column, Eluent of 2-4% Ethyl acetate/Petroleum ether gradient @120 mL/min) to give Compound 18 (7.6 g, 12.22 mmol, 67.75% yield, 96.11% purity) as a colorless oil.

LCMS [M−100+1]⁺=498.5

¹H NMR (400 MHz, CHLOROFORM-d) δ=4.28-4.04 (m, 4H), 3.66-3.35 (m, 4H), 2.41-2.21 (m, 3H), 1.68-1.59 (m, 7H), 1.47 (s, 9H), 1.38-1.21 (m, 26H), 1.15 (br d, J=6.4 Hz, 2H), 0.96-0.80 (m, 12H)

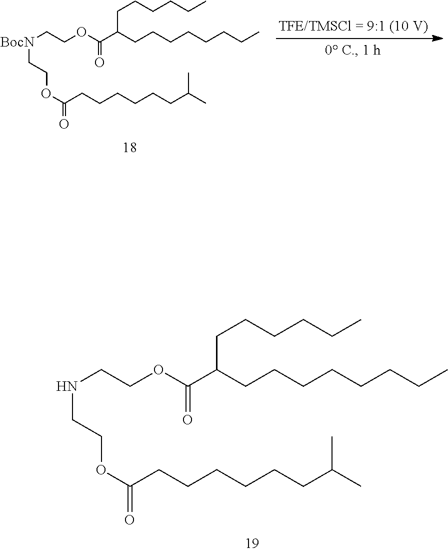 [SMILES: CCCCCCCCC(CCCCCC)C(=O)OCCN(CCOC(=O)CCCCCCC(C)C)C(=O)OC(C)(C)C ; CCCCCCCCC(CCCCCC)C(=O)OCCNCCOC(=O)CCCCCCC(C)C]

To a solution of Compound 18 (6.83 g, 11.42 mmol, 1 eq.) in TFE (61.5 mL) was added TMSCI (6.83 mL) at 0° C. The mixture was stirred at 0° C. for 1 hour. TLC (Petroleum ether:Ethyl acetate=5:1, R_{f}=5:1) indicated Compound 18 was consumed completely and one new spot formed. The reaction mixture was concentrated under reduced pressure to give a residue (HCl salt). The residue was dissolved with DCM (50 mL) and basified with saturated NaHCO₃ (45 mL) to pH=7 at 25° C. The mixture was poured into separatory funnel and separated. The aqueous layer was extracted with DCM (30 mL×2). The combined organic layers were dried over Na₂SO₄, filtered, and the filtrate was concentrated to give Compound 19 (5.53 g, 10.88 mmol, 95.27% yield, 97.96% purity, Free) as a colorless oil.

LCMS [M+1]⁺=498.5

¹H NMR (400 MHz, CHLOROFORM-d) δ=4.19 (q, J=6.0 Hz, 4H), 2.90 (t, J=5.6 Hz, 4H), 2.39-2.25 (m, 3H), 1.61-1.38 (m, 7H), 1.37-1.21 (m, 26H), 1.15 (br d, J=6.8 Hz, 2H), 0.98-0.73 (m, 12H).

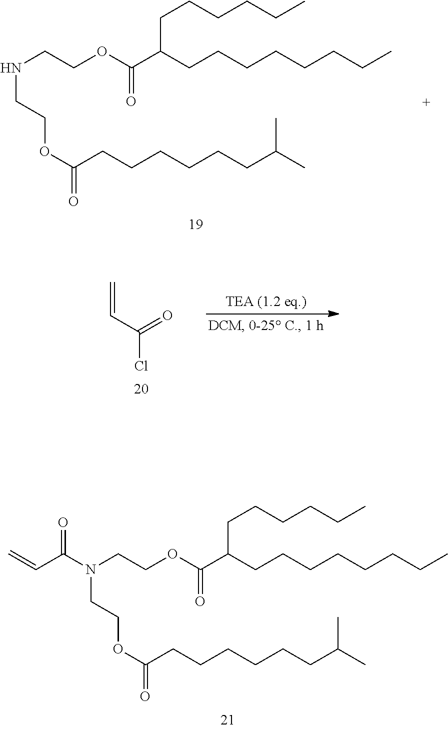

To a solution of Compound 19 (5.53 g, 11.11 mmol, 1 eq.) in DCM (55.3 mL) was added TEA (1.35 g, 13.33 mmol, 1.86 mL, 1.2 eq.) and Compound 20 (1.21 g, 13.33 mmol, 1.08 mL, 1.2 eq.) at 0° C. The mixture was stirred at 25° C. for 1 hour. TLC (Petroleum ether:Ethyl acetate=1:1, R_{f}=0.87) indicated Compound 19 was consumed completely and new spots formed. The reaction mixture was diluted with saturated NH₄Cl (50 mL) and extracted with DCM (50 mL×2). The combined organic layers were washed with brine (30 mL), dried over Na₂SO₄, filtered and concentrated under reduced pressure to give a residue (6.8 g). The residue was purified by flash silica gel chromatography (ISCO®; 80 g SepaFlash® Silica Flash Column, 2-6 and 6-10% Ethyl acetate/Petroleum ether gradient @150 mL/min) to give Compound 21 (4.58 g, 8.04 mmol, 72.36% yield, 96.86% purity) as a light-yellow oil.

LCMS [M+1]⁺=552.5

¹H NMR (400 MHz, CHLOROFORM-d) δ=6.74-6.54 (m, 1H), 6.40 (dd, J=2.0, 16.8 Hz, 1H), 5.74 (br d, J=10.4 Hz, 1H), 4.32-4.13 (m, 4H), 3.76-3.61 (m, 4H), 2.38-2.24 (m, 3H), 1.61-1.40 (m, 6H), 1.42 (br d, J=7.6 Hz, 1H), 1.36-1.20 (m, 26H), 1.15 (br d, J=6.8 Hz, 2H), 0.95-0.80 (m, 12H

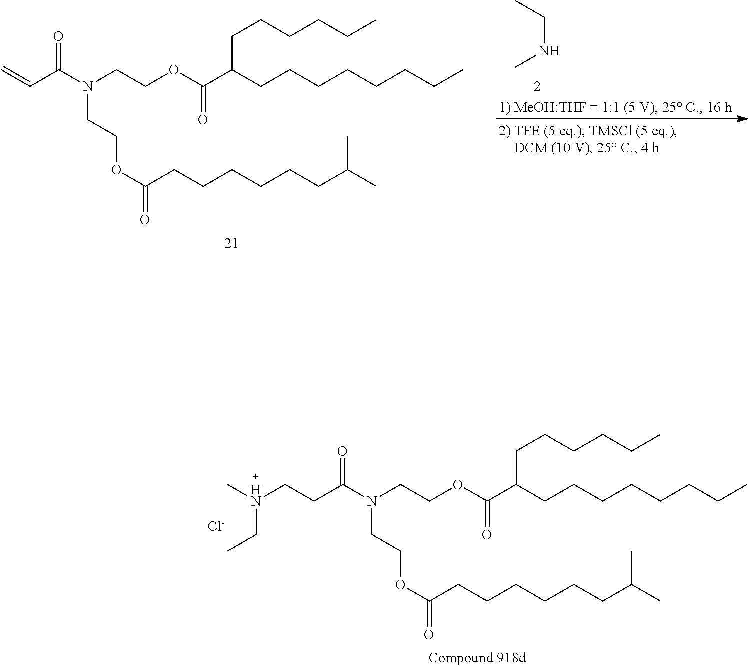

To a solution of Compound 21 (800 mg, 1.45 mmol, 1 eq.) in MeOH (2 mL) and THF (2 mL) was added Compound 2 (257.07 mg, 4.35 mmol, 373.66 μL, 3 eq.). The mixture was stirred at 25° C. for 16 hours. TLC (Petroleum ether:Ethyl acetate=5:1, R_{f}=0.04) indicated trace Compound 21 remained and new spots formed. The reaction mixture was concentrated under reduced pressure to give the crude product. The crude product was purified by column chromatography (SiO₂, Petroleum ether:Ethyl acetate=3:1 to 1:1) to give Compound 918d (155 mg, 249.42 μmol, 15.24% yield, 98.31% purity, Free) as a colorless oil.

LCMS [M+1]⁺=611.6

1H NMR (400 MHz, CHLOROFORM-d) δ=4.25-4.19 (m, 4H), 3.69-3.59 (m, 4H), 2.80-2.72 (m, 2H), 2.64-2.45 (m, 4H), 2.38-2.25 (m, 6H), 1.63-1.43 (m, 7H), 1.36-1.23 (m, 26H), 1.20-1.07 (m, 5H), 0.93-0.85 (m, 12H)

A mixture of TFE (126.90 mg, 1.27 mmol, 91.23 μL, 5 eq.) and TMSCI (137.81 mg, 1.27 mmol, 161.00 μL, 5 eq.) was stirred at 25° C. for 1 hour under N₂ atmosphere. Then, Compound 918d (155 mg, 253.70 μmol, 1 eq.) in DCM (1.5 mL) was added to the mixture. The resulting mixture was stirred at 25° C. for 3 hours under N₂ atmosphere. The reaction mixture was concentrated under reduced pressure to give Compound 918d (153 mg, 236.15 μmol, 93.08% yield, 98.80% purity, 0.8 HCl) as a colorless gum.

LCMS [M+1]⁺=611.6

¹H NMR (400 MHz, CHLOROFORM-d) δ=12.54-12.38 (m, 1H), 4.28-4.16 (m, 4H), 3.74-3.56 (m, 4H), 3.53-3.41 (m, 1H), 3.38-2.98 (m, 5H), 2.75 (d, J=4.8 Hz, 3H), 2.33 (td, J=7.6, 12.0 Hz, 3H), 1.66-1.59 (m, 3H), 1.55-1.42 (m, 7H), 1.34-1.20 (m, 26H), 1.18-1.12 (m, 2H), 0.93-0.83 (m, 12H)

### General Procedure for Preparation of Compound 918e:

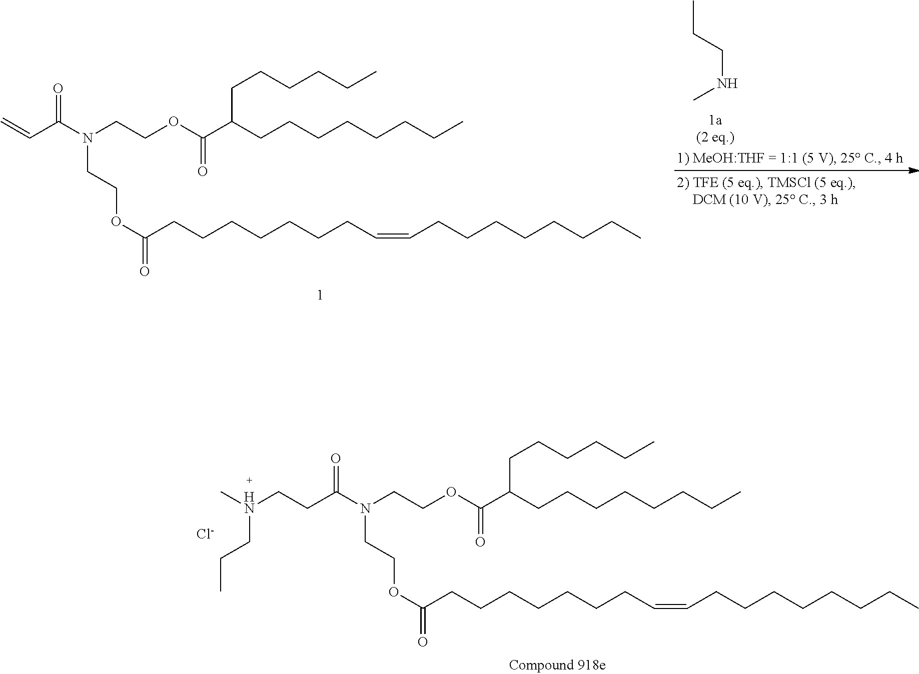

To a solution of Compound 1 (1 g, 1.51 mmol, 1 eq.) in THF (2.5 mL) and MeOH (2.5 mL) was added Compound 1a (220.94 mg, 3.02 mmol, 309.88 μL, 2 eq.). The mixture was stirred at 25° C. for 4 hours. LCMS showed Compound 1 was consumed completely and one main peak with desired MS was detected. The reaction mixture was concentrated to give a residue. The residue was purified by column chromatography (SiO₂, Petroleum ether:Ethyl acetate=75:25 to 0:100) to give Compound 918e (6.2 g, 7.94 mmol, 46.18% yield, 98.2% purity) as a colorless oil.

LCMS [M+1]⁺=735.6

¹H NMR (400 MHz, CHLOROFORM-d) δ=5.44-5.27 (m, 2H), 4.26-4.15 (m, 4H), 3.69-3.56 (m, 4H), 2.89-2.51 (m, 4H), 2.45-2.24 (m, 8H), 2.06-1.96 (m, 4H), 1.58-1.39 (m, 8H), 1.36-1.21 (m, 40H), 0.96-0.82 (m, 12H)

To a solution of TMSCI (295.56 mg, 2.72 mmol, 345.28 μL, 5 eq.) was added TFE (272.15 mg, 2.72 mmol, 195.65 μL, 5 eq.) at 20° C. for 1 hour. Then, Compound 918e (400 mg, 544.09 μmol, 1 eq.) in DCM (4 mL) was added at 20° C. The resulting mixture was stirred at 20° C. for 3 hours. The reaction mixture was concentrated to give Compound 918e (106.21 mg, 135.82 μmol, 24.96% yield, 98.21% purity, 0.9 HCl) as a colorless gum.

LCMS [M+1]⁺=735.7

¹H NMR (400 MHz, CHLOROFORM-d) δ=12.58-12.22 (m, 1H), 5.41-5.29 (m, 2H), 4.29-4.14 (m, 4H), 3.75-3.56 (m, 4H), 3.53-3.41 (m, 1H), 3.38-3.27 (m, 1H), 3.25-3.14 (m, 2H), 3.11-2.99 (m, 1H), 2.97-2.83 (m, 1H), 2.76 (d, J=4.8 Hz, 3H), 2.32-2.32 (m, 1H), 2.33 (td, J=7.6, 11.6 Hz, 2H), 2.02 (br t, J=2.8 Hz, 4H), 1.98-1.81 (m, 2H), 1.62 (br s, 2H), 1.58-1.52 (m, 2H), 1.49-1.40 (m, 2H), 1.37-1.20 (m, 40H), 1.04 (t, J=7.2 Hz, 3H), 0.89 (t, J=6.8 Hz, 9H).

### General Procedure for Preparation of Compound 918f:

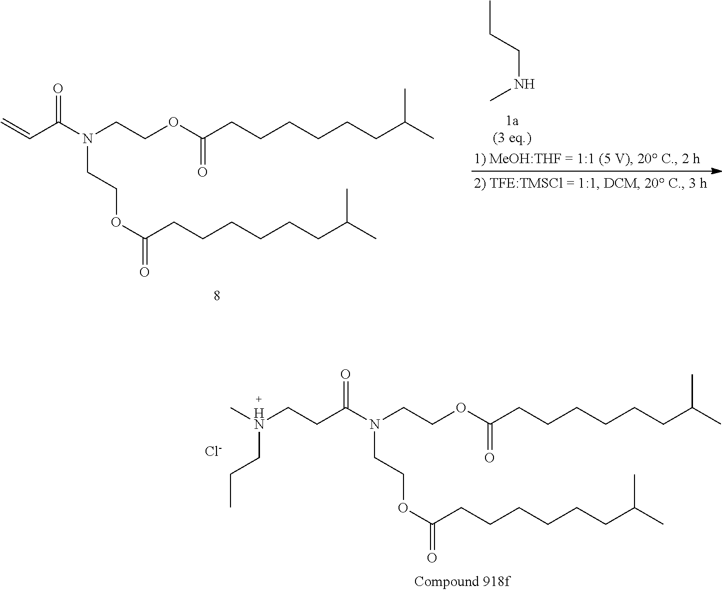

To a solution of Compound 8 (600 mg, 1.28 mmol, 1 eq.) in MeOH (1.5 mL) and THF (1.5 mL) was added Compound 1a (281.49 mg, 3.85 mmol, 394.79 μL, 3 eq.). The mixture was stirred at 20° C. for 2 hours. TLC (Petroleum ether:Ethyl acetate=5:1, R_{f}=0) indicated Compound 8 was consumed completely and one new spot formed. The reaction mixture was concentrated under reduced pressure to give a residue. The residue was purified by column chromatography (SiO₂, Petroleum ether:Ethyl acetate=80:20 to 0:1) to give Compound 918f (530 mg, 970.20 μmol, 75.62% yield, 99% purity, Free) as a white solid.

LCMS [M+1]⁺=541.5

¹H NMR (400 MHz, CHLOROFORM-d) δ=4.28-4.14 (m, 4H), 3.72-3.53 (m, 4H), 2.79-2.69 (m, 2H), 2.57 (br t, J=7.2 Hz, 2H), 2.38-2.28 (m, 6H), 2.26 (s, 3H), 1.62-1.56 (m, 4H), 1.56-1.45 (m, 4H), 1.28 (br s, 12H), 1.19-1.12 (m, 4H), 0.95-0.83 (m, 15H)

A solution of TMSCI (2.5 mL) and TFE (2.5 mL) in DCM (5 mL) was added Compound 918f (530 mg, 980.00 μmol, 1 eq.) at 20° C. The mixture was stirred at 20° C. for 3 hours. The reaction mixture was concentrated under reduced pressure to give Compound 918f (304 mg, 523.97 μmol, 53.47% yield, 98.87% purity, 0.9 HCl) as a light yellow oil.

LCMS [M+1]⁺=541.5

¹H NMR (400 MHz, CHLOROFORM-d) δ=12.36 (br s, 1H), 4.31-4.15 (m, 4H), 3.68 (br s, 2H), 3.64-3.54 (m, 2H), 3.52-3.41 (m, 1H), 3.39-3.27 (m, 1H), 3.25-3.14 (m, 2H), 3.10-2.98 (m, 1H), 2.91 (br dd, J=6.0, 12.0 Hz, 1H), 2.75 (d, J=4.4 Hz, 3H), 2.31 (td, J=7.6, 11.2 Hz, 4H), 2.01-1.84 (m, 2H), 1.65-1.56 (m, 4H), 1.55-1.44 (m, 2H), 1.27 (br s, 12H), 1.15 (br d, J=6.0 Hz, 4H), 1.03 (t, J=7.2 Hz, 3H), 0.86 (d, J=6.4 Hz, 12H)

### General Procedure for Preparation of Compound 918g:

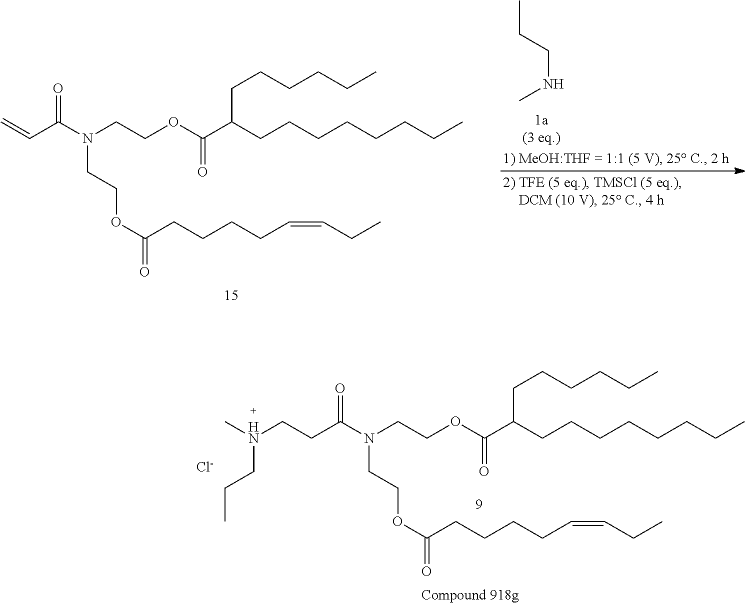

To a solution of Compound 15 (800 mg, 1.49 mmol, 1 eq.) in MeOH (2 mL) and THF (2 mL) was added Compound 1a (327.60 mg, 4.48 mmol, 459.47 μL, 3 eq.). The mixture was stirred at 25° C. for 2 hours. TLC (Petroleum ether:Ethyl acetate=3:1) showed Compound was consumed completely and one major new spot was formed. The reaction mixture was concentrated under reduced pressure to give a residue. The residue was purified by column chromatography (SiO₂, Petroleum ether:Ethyl acetate=5:1 to 1:1) to give Compound 918g (200 mg, 322.79 μmol, 21.62% yield, 98.28% purity, Free) as a colorless oil.

LCMS [M+1]⁺=609.5

1H NMR (400 MHz, CHLOROFORM-d) δ=5.44-5.26 (m, 2H), 4.26-4.17 (m, 4H), 3.71-3.56 (m, 4H), 3.12-2.58 (m, 4H), 2.57-2.21 (m, 8H), 2.10-1.97 (m, 4H), 1.70-1.61 (m, 4H), 1.51-1.31 (m, 6H), 1.26 (s, 20H), 1.01-0.84 (m, 12H)

A mixture of TFE (164.29 mg, 1.64 mmol, 118.11 μL, 5 eq.) and TMSCI (178.41 mg, 1.64 mmol, 208.43 μL, 5 eq.) was degassed and purged with N₂ 3 times; the mixture was stirred at 25° C. for 1 hour under N₂ atmosphere. Then, Compound 918g (200 mg, 328.44 μmol, 1 eq) in DCM (2 mL) was added, and the mixture was stirred at 25° C. for 3 hours under N₂ atmosphere. The reaction mixture was concentrated under reduced pressure to give Compound 918g (200 mg, 307.13 μmol, 93.51% yield, 98.55% purity, 0.9 HCl) as a colorless oil.

LCMS [M+1]⁺=609.6

¹H NMR (400 MHz, CHLOROFORM-d) δ=12.48-12.28 (m, 1H), 5.44-5.22 (m, 2H), 4.33-4.13 (m, 4H), 3.76-3.55 (m, 4H), 3.52-3.27 (m, 2H), 3.19 (br s, 2H), 3.11-2.84 (m, 2H), 2.76 (br d, J=4.4 Hz, 3H), 2.39-2.27 (m, 3H), 2.10-1.81 (m, 6H), 1.68-1.61 (m, 2H), 1.55 (br s, 2H), 1.49-1.35 (m, 4H), 1.31-1.20 (m, 20H), 1.03 (t, J=7.2 Hz, 3H), 0.96 (t, J=7.6 Hz, 3H), 0.91-0.85 (m, 6H)

### General Procedure for Purification of Compound 918h:

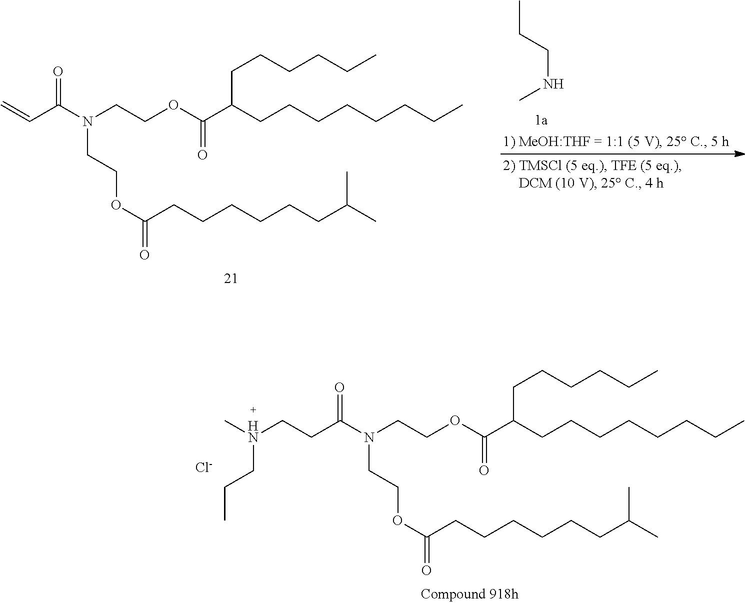

A solution of Compound 21 (0.8 g, 1.45 mmol, 1 eq.) and Compound 1a (212.05 mg, 2.90 mmol, 297.41 μL, 2 eq.) in THF (4 mL) and MeOH (4 mL) was stirred at 25° C. for 5 hours. TLC (Petroleum ether:Ethyl acetate=5:1, R_{f}=0.42) indicated Compound 21 was consumed completely and one new spot formed. The reaction mixture was concentrated under reduced pressure to give a residue. The residue was purified by column chromatography (SiO₂, Petroleum ether:Ethyl acetate=100:1 to 70:30) to give Compound 918h (735 mg, 1.16 mmol, 80.35% yield, 99.05% purity, Free) as a light-yellow oil.

LCMS [M+1]⁺=625.6

¹H NMR (400 MHz, CHLOROFORM-d) δ=4.31-4.11 (m, 4H), 3.72-3.51 (m, 4H), 2.86-2.69 (m, 2H), 2.59 (br d, J=5.6 Hz, 2H), 2.42-2.19 (m, 8H), 1.61-1.38 (m, 9H), 1.34-1.20 (m, 26H), 1.18-1.09 (m, 2H), 1.00-0.76 (m, 15H).

A solution of TFE (588.26 mg, 5.88 mmol, 422.90 μL, 5 eq.) and TMSCI (638.83 mg, 5.88 mmol, 746.30 μL, 5 eq.) was stirred at 25° C. for 1 hour. Then, Compound 918h (735 mg, 1.18 mmol, 1 eq.) in DCM (7 mL) was added. The resulting mixture was stirred at 25° C. for 3 hours. The reaction mixture was concentrated under reduced pressure to give Compound 918h (731 mg, 1.11 mmol, 94.06% yield, 99.54% purity, 0.9HCl) as a yellow oil.

LCMS [M+1]⁺=625.6

¹H NMR (400 MHz, CHLOROFORM-d) δ=12.57-12.25 (m, 1H), 4.36-4.09 (m, 4H), 3.69 (br s, 2H), 3.64-3.54 (m, 2H), 3.52-3.42 (m, 1H), 3.40-3.28 (m, 1H), 3.28-3.11 (m, 2H), 3.10-2.99 (m, 1H), 2.96-2.86 (m, 1H), 2.76 (d, J=4.8 Hz, 3H), 2.33 (td, J=7.6, 11.2 Hz, 3H), 2.03-1.81 (m, 2H), 1.59-1.40 (m, 7H), 1.35-1.20 (m, 26H), 1.18-1.12 (m, 2H), 1.04 (t, J=7.2 Hz, 3H), 0.94-0.81 (m, 12H)

### General Procedure for Preparation of Compound 918i:

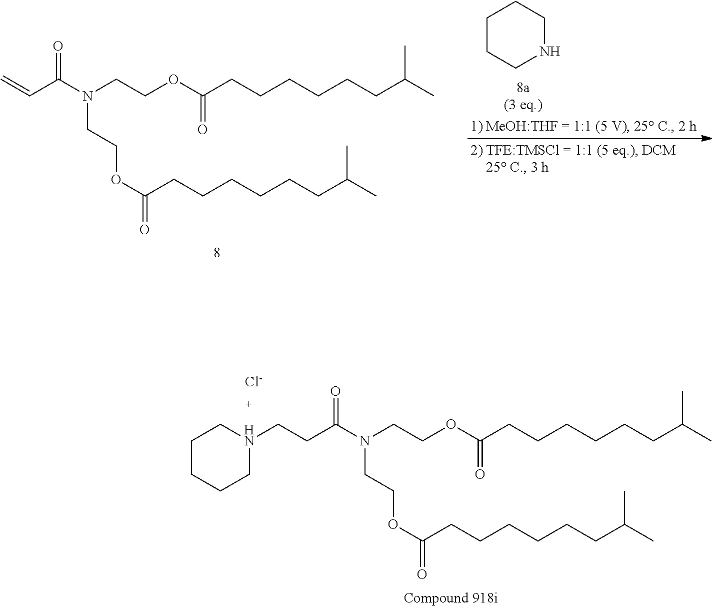

To a solution of Compound 8 (600.00 mg, 1.28 mmol, 1 eq.) in MeOH (1.5 mL) and THF (1.5 mL) was added Compound 8a (327.71 mg, 3.85 mmol, 380.09 μL, 3 eq.). The mixture was stirred at 20° C. for 2 hours. TLC (Petroleum ether:Ethyl acetate=5:1, R_{f}=0) indicated Compound 8 was consumed completely and one new spot formed. The reaction mixture was concentrated under reduced pressure to give a residue. The residue was purified by column chromatography (SiO₂, Petroleum ether:Ethyl acetate=80:20 to 0:1) to give Compound 918i (500 mg, 891.78 μmol, 69.51% yield, 98.6% purity, Free) as a white solid.

LCMS [M+1]⁺=553.5

¹H NMR (400 MHz, CHLOROFORM-d) δ=4.20 (q, J=5.6 Hz, 4H), 3.62 (q, J=5.6 Hz, 4H), 2.74-2.63 (m, 2H), 2.63-2.52 (m, 2H), 2.50-2.36 (m, 4H), 2.34-2.24 (m, 4H), 1.67-1.56 (m, 8H), 1.51 (td, J=6.8, 13.2 Hz, 2H), 1.47-1.39 (m, 2H), 1.28 (br s, 12H), 1.20-1.11 (m, 4H), 0.86 (d, J=6.8 Hz, 12H).

A solution of TFE (2.5 mL) and TMSCI (2.5 mL) in DCM (5 mL) was added Compound 918i (500 mg, 904.44 μmol, 1 eq.). The mixture was stirred at 20° C. for 3 hours. The reaction mixture was concentrated under reduced pressure to give Compound 918i (222 mg, 375.62 μmol, 41.53% yield, 99.09% purity, 0.9 HCl) as a light yellow solid.

LCMS [M+1]⁺=553.5

¹H NMR (400 MHz, CHLOROFORM-d) δ=12.12 (br s, 1H), 4.21 (td, J=5.6, 11.2 Hz, 4H), 3.69 (br t, J=5.6 Hz, 2H), 3.60 (t, J=5.6 Hz, 2H), 3.49 (br d, J=12.0 Hz, 2H), 3.34 (br d, J=5.2 Hz, 2H), 3.28-3.17 (m, 2H), 2.77-2.57 (m, 2H), 2.37-2.17 (m, 6H), 1.97-1.81 (m, 3H), 1.64-1.56 (m, 4H), 1.51 (td, J=6.8, 13.2 Hz, 2H), 1.46-1.37 (m, 1H), 1.28 (br d, J=2.0 Hz, 12H), 1.20-1.10 (m, 4H), 0.86 (d, J=6.4 Hz, 12H)

### General Procedure for Preparation of Compound 918j:

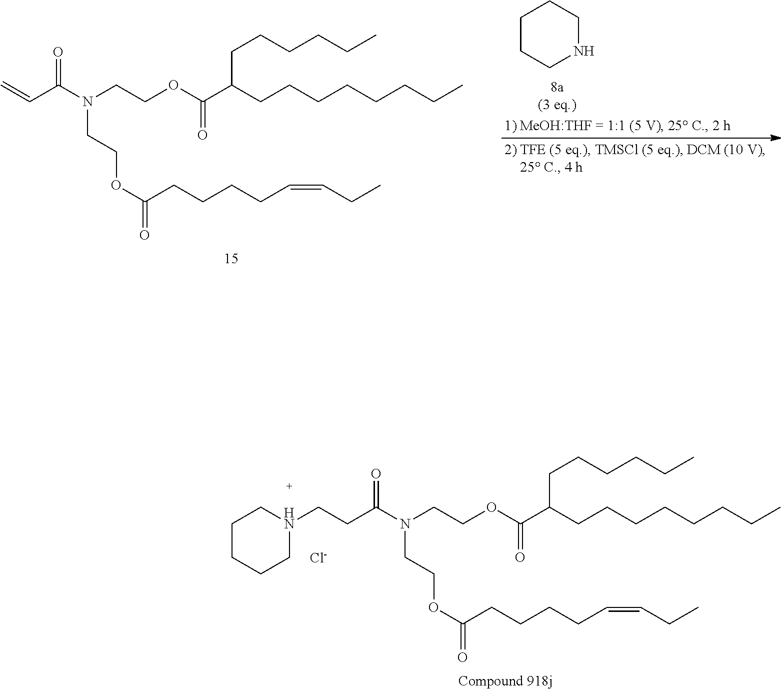

To a solution of Compound 15 (800 mg, 1.49 mmol, 1 eq.) in MeOH (2 mL) and THF (2 mL) was added Compound 8a (381.40 mg, 4.48 mmol, 442.36 μL, 3 eq.). The mixture was stirred at 25° C. for 2 hours. TLC (Petroleum ether:Ethyl acetate=3:1) showed Compound 15 was consumed completely and one major new spot was formed. The reaction mixture was concentrated under reduced pressure to give a residue. The residue was purified by column chromatography (SiO₂, Petroleum ether:Ethyl acetate=5:1 to 1:1) to give Compound 918j (710 mg, 1.13 mmol, 75.87% yield, 99.07% purity, Free) as a light yellow oil.

LCMS [M+1]⁺=621.5

¹H NMR (400 MHz, CHLOROFORM-d) δ=5.44-5.25 (m, 2H), 4.26-4.16 (m, 4H), 3.69-3.56 (m, 4H), 2.96-2.38 (m, 8H), 2.36-2.29 (m, 3H), 2.09-2.00 (m, 4H), 1.71-1.64 (m, 4H), 1.57-1.34 (m, 10H), 1.26 (s, 20H), 0.96 (t, J=7.6 Hz, 3H), 0.88 (t, J=6.8 Hz, 6H)

A mixture of CF₃CH₂OH (322.22 mg, 3.22 mmol, 231.65 μL, 5 eq.) and TMSCI (349.93 mg, 3.22 mmol, 408.79 μL, 5 eq.) was stirred at 25° C. for 1 hour under N₂ atmosphere. Then, Compound 918j (400 mg, 644.18 μmol, 1 eq.) in DCM (4 mL) was added, and the mixture was stirred at 25° C. for 3 hours under N₂ atmosphere. The reaction mixture was concentrated under reduced pressure to give Compound 918j (370 mg, 561.15 μmol, 87.11% yield, 99.15% purity, 0.9 HCl) as a colorless gum.

LCMS [M+1]⁺=621.6

¹H NMR (400 MHz, CHLOROFORM-d) δ=12.29-12.04 (m, 1H), 5.44-5.24 (m, 2H), 4.28-4.15 (m, 4H), 3.73-3.56 (m, 4H), 3.49 (br d, J=11.2 Hz, 2H), 3.34 (br d, J=4.0 Hz, 2H), 3.23 (br d, J=6.4 Hz, 2H), 2.73-2.60 (m, 2H), 2.38-2.19 (m, 5H), 2.10-1.99 (m, 4H), 1.97-1.82 (m, 3H), 1.68-1.61 (m, 2H), 1.57-1.51 (m, 2H), 1.49-1.36 (m, 5H), 1.33-1.22 (m, 20H), 0.96 (t, J=7.6 Hz, 3H), 0.88 (t, J=6.8 Hz, 6H)

### General Procedure for Preparation of Compound 918k:

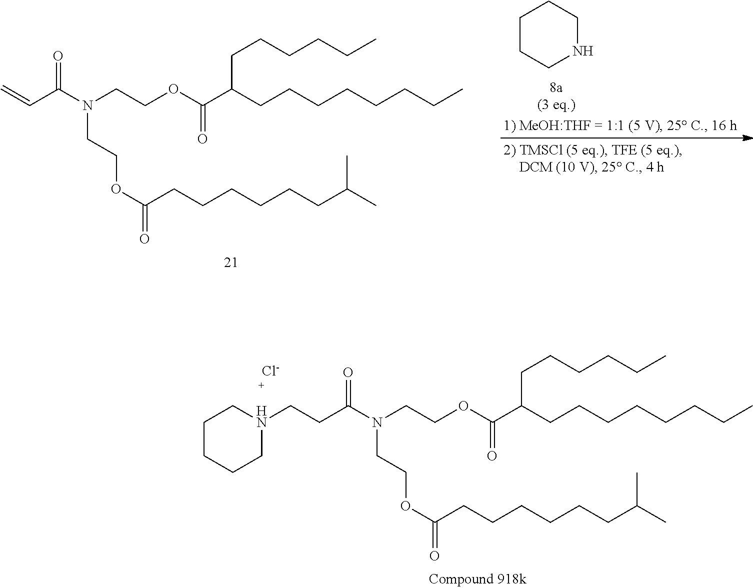

To a solution of Compound 21 (800 mg, 1.45 mmol, 1 eq.) in MeOH (2 mL) and THF (2 mL) was added Compound 8a (370.31 mg, 4.35 mmol, 3 eq.) at 25° C. The mixture was stirred at 25° C. for 16 hours. LCMS showed Compound 21 was consumed completely and desired MS was detected. The reaction mixture was concentrated under reduced pressure to give a residue. The residue was purified by column chromatography (SiO₂, Petroleum ether:Ethyl acetate=10:1 to 1:1) to give Compound 918k (670 mg, 1.05 mmol, 99.17% purity, Free) as a light yellow oil.

LCMS [M+1]⁺=637.6

¹H NMR (400 MHz, CHLOROFORM-d) δ=4.32-4.12 (m, 4H), 3.70-3.54 (m, 4H), 2.78-2.54 (m, 4H), 2.44 (br s, 3H), 2.37-2.25 (m, 3H), 1.71-1.59 (m, 8H), 1.52 (br dd, J=6.4, 13.2 Hz, 2H), 1.45 (br d, J=3.2 Hz, 4H), 1.33-1.20 (m, 26H), 1.18-1.09 (m, 2H), 0.97-0.80 (m, 12H)

A mixture of TMSCI (571.36 mg, 5.26 mmol, 5 eq.) in TFE (526.12 mg, 5.26 mmol, 5 eq.) was stirred at 25° C. for 1 hour under N₂ atmosphere. Then, Compound 918k (670 mg, 1.05 mmol, 1 eq.) in DCM (6.5 mL) was added at 25° C. The mixture was stirred at 25° C. for 3 hours. The reaction mixture was concentrated under reduced pressure to give Compound 918k (674 mg, 1.00 mmol, 100% purity) as a light yellow oil.

LCMS [M+1]⁺=637.6

¹H NMR (400 MHz, CHLOROFORM-d) δ=12.32-12.09 (m, 1H), 4.30-4.14 (m, 4H), 3.69 (br d, J=2.8 Hz, 2H), 3.61 (q, J=6.0 Hz, 2H), 3.49 (br d, J=11.2 Hz, 2H), 3.34 (br d, J=5.2 Hz, 2H), 3.27-3.15 (m, 2H), 2.80-2.56 (m, 2H), 2.41-2.17 (m, 5H), 1.98-1.80 (m, 3H), 1.67-1.60 (m, 2H), 1.57-1.38 (m, 6H), 1.35-1.20 (m, 26H), 1.16 (br d, J=6.0 Hz, 2H), 0.97-0.79 (m, 12H)

### General Procedure for Preparation of Compound 918l:

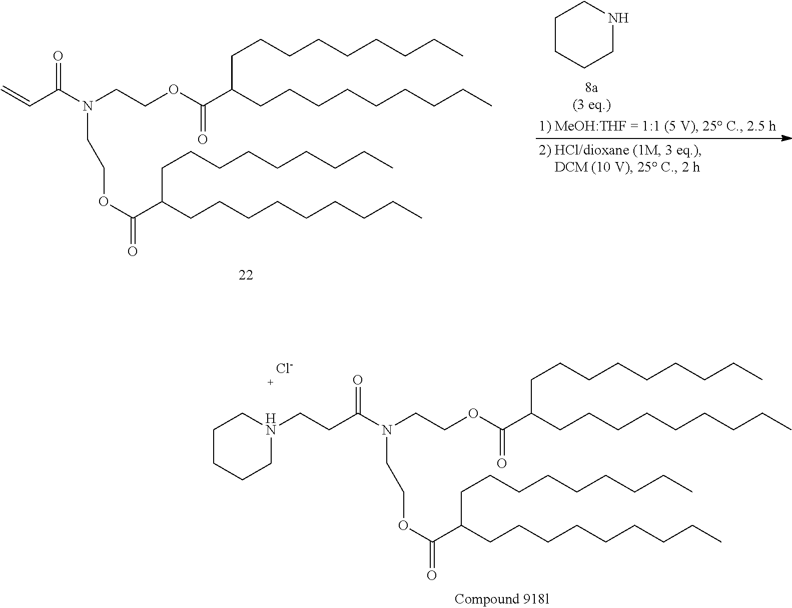

To a solution of Compound 22 (500 mg, 668.26 μmol, 1 eq.) in MeOH (1.25 mL) and THF (1.25 mL) was added Compound 8a (170.70 mg, 2.00 mmol, 197.98 μL, 3 eq.). The mixture was stirred at 25° C. for 2.5 hours. LCMS showed Compound 22 was consumed completely and one main peak with desired MS was detected. TLC (Petroleum ether:Ethyl acetate=5:1 and DCM:MeOH=10:1, R_{f}:R1=0.56, R_{f}:P=0.49) indicated Compound 22 was consumed completely and new spots formed. The reaction mixture was concentrated under reduced pressure to give a residue. The residue was purified by column chromatography (SiO₂, Petroleum ether:Ethyl acetate=10:1 to 3:1) to give Compound 918l (250 mg, 297.11 μmol, 44.46% yield, 99.04% purity) as a colorless oil.

LCMS [M+1]⁺=833.8

¹H NMR (400 MHz, CHLOROFORM-d) δ=4.20 (dt, J=3.6, 5.6 Hz, 4H), 3.67-3.56 (m, 4H), 2.78-2.57 (m, 4H), 2.46 (br s, 4H), 2.35-2.26 (m, 2H), 1.63-1.52 (m, 8H), 1.50-1.40 (m, 6H), 1.31-1.19 (m, 56H), 0.94-0.81 (m, 12H)

To a solution of Compound 918l (250 mg, 299.99 μmol, 1 eq.) in DCM (2.5 mL) was added HCl/dioxane (1 M, 299.99 μL, 1 eq.). The mixture was stirred at 25° C. for 2 hours. The mixture was concentrated and lyophilized to give Compound 918l (101.51 mg, 116.83 μmol, 38.94% yield, 99.27% purity, 0.8 HCl) as a light yellow gum.

LCMS [M+1]⁺=833.8

¹H NMR (400 MHz, CHLOROFORM-d) δ=12.26-12.01 (m, 1H), 4.21 (td, J=5.6, 17.6 Hz, 4H), 3.69 (br t, J=5.6 Hz, 2H), 3.60 (t, J=5.6 Hz, 2H), 3.50 (br d, J=11.2 Hz, 2H), 3.34 (br d, J=5.2 Hz, 2H), 3.23 (br d, J=6.0 Hz, 2H), 2.73-2.60 (m, 2H), 2.37-2.19 (m, 4H), 1.96-1.80 (m, 3H), 1.55 (br s, 4H), 1.50-1.40 (m, 5H), 1.33-1.22 (m, 56H), 0.88 (t, J=6.8 Hz, 12H)

### General Procedure for Preparation of Compound 918m:

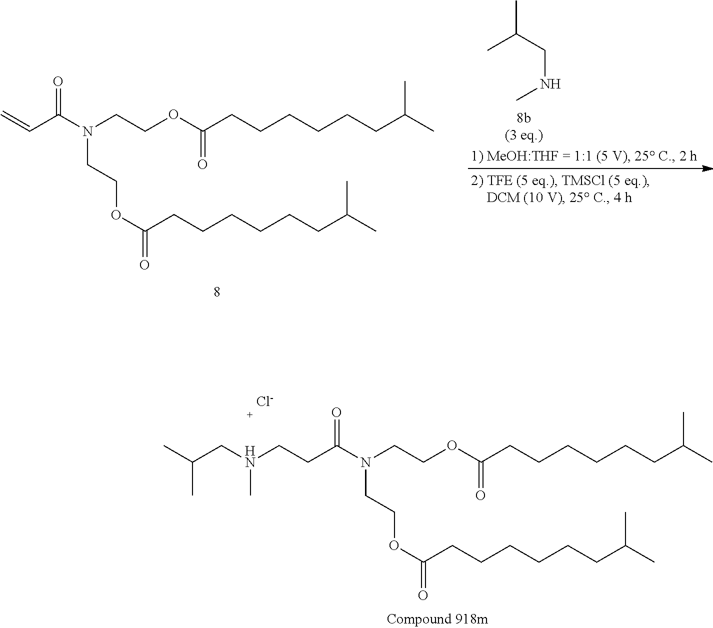

To a solution of Compound 8 (600 mg, 1.28 mmol, 1 eq.) in MeOH (1.5 mL) and THF (1.5 mL) was added Compound 8b (335.47 mg, 3.85 mmol, 3 eq.). The mixture was stirred at 25° C. for 2 hours. TLC (Dichloromethane:Methanol=10:1) showed Compound 8 was consumed completely and one major new spot was formed. The reaction mixture was concentrated under reduced pressure to give a residue. The residue was purified by column chromatography (SiO₂, Petroleum ether:Ethyl acetate=5:1 to 1:1) to give Compound 918m (300 mg, 531.29 μmol, 41.41% yield, 98.26% purity) as a colorless oil.

LCMS [M+1]⁺=555.5

1H NMR (400 MHz, CHLOROFORM-d) δ=4.21 (t, J=5.6 Hz, 4H), 3.63 (td, J=5.6, 11.6 Hz, 4H), 2.79-2.47 (m, 4H), 2.36-2.19 (m, 7H), 2.11 (br d, J=4.8 Hz, 2H), 1.82-1.69 (m, 1H), 1.63 (br s, 2H), 1.58 (br s, 2H), 1.56-1.45 (m, 2H), 1.28 (br s, 12H), 1.20-1.09 (m, 4H), 0.95-0.80 (m, 18H).

A mixture of CF₃CH₂OH (270.45 mg, 2.70 mmol, 194.43 μL, 5 eq.) and TMSCI (293.71 mg, 2.70 mmol, 343.12 μL, 5 eq.) was stirred at 25° C. for 1 hour under N₂ atmosphere. Then, Compound 918m (300 mg, 540.69 μmol, 1 eq.) in DCM (3 mL) was added and stirred at 25° C. for 3 hours under N₂ atmosphere. The reaction mixture was concentrated under reduced pressure to give a residue. The residue was purified by column chromatography by column chromatography (SiO₂, Dichloromethane:Methanol=20:1 to 10:1) to give Compound 918m (210 mg, 361.84 μmol, 66.92% yield, 100% purity, 0.7HCl) as a colorless gum.

LCMS [M+1]⁺=555.5

¹H NMR (400 MHz, CHLOROFORM-d) δ=12.35-11.00 (m, 1H), 4.22 (td, J=5.6, 12.8 Hz, 4H), 3.74-3.56 (m, 4H), 3.42-3.07 (m, 4H), 2.84-2.62 (m, 5H), 2.38-2.25 (m, 4H), 2.20-2.05 (m, 1H), 1.79-1.66 (m, 3H), 1.58-1.44 (m, 3H), 1.28 (br d, J=2.0 Hz, 13H), 1.19-1.11 (m, 9H), 0.86 (d, J=6.4 Hz, 12H)

### General Procedure for Preparation of Compound 918n:

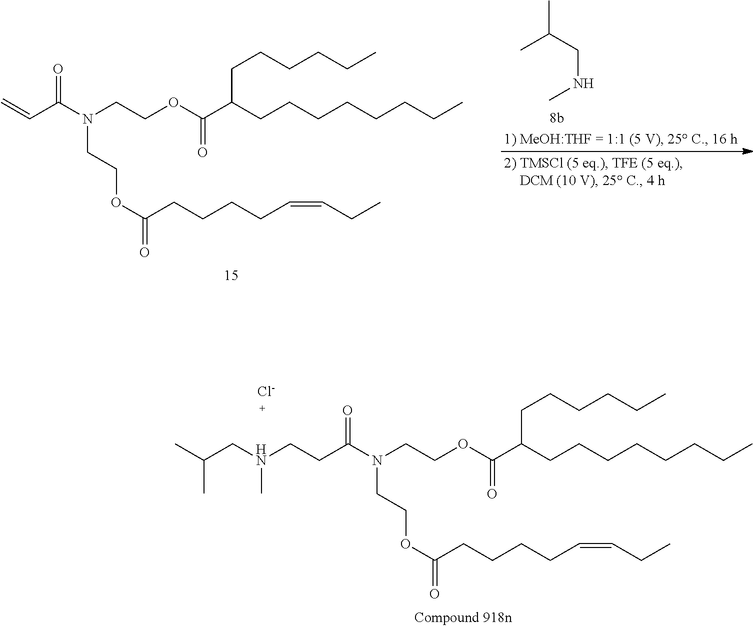

To a solution of Compound 7 (800 mg, 1.49 mmol, 1 eq.) in MeOH (2 mL) and THF (2 mL) was added Compound 7A (390.43 mg, 4.48 mmol, 3 eq.) at 25° C. The mixture was stirred at 25° C. for 16 hours. LCMS showed Compound 7 was consumed completely and desired MS was detected. The reaction mixture was concentrated under reduced pressure to give a residue. The residue was purified by column chromatography (SiO₂, Petroleum ether:Ethyl acetate=10:1 to 1:1) to give Compound 918n (530 mg, 850.78 mmol, 98.19% purity, Free) as a light yellow oil.

LCMS [M+1]⁺=623.6

¹H NMR (400 MHz, CHLOROFORM-d) δ=5.48-5.23 (m, 2H), 4.32-4.17 (m, 4H), 3.73-3.55 (m, 4H), 2.72 (br s, 2H), 2.57 (br s, 2H), 2.39-2.29 (m, 3H), 2.27-2.20 (m, 3H), 2.12 (br s, 2H), 2.09-1.95 (m, 4H), 1.85-1.72 (m, 1H), 1.70-1.55 (m, 5H), 1.47-1.35 (m, 4H), 1.25 (s, 19H), 0.96 (t, J=7.6 Hz, 3H), 0.92-0.84 (m, 12H).

A mixture of TFE (152.56 mg, 1.52 mmol, 109.68 μL, 5 eq.) and TMSCI (165.68 mg, 1.52 mmol, 193.55 μL, 5 eq.) was stirred at 25° C. for 1 hour. Then, Compound 918n (190 mg, 305.00 μmol, 1 eq.) in DCM (1.9 mL) was added at 25° C. The mixture was stirred at 25° C. for 3 hours. The reaction mixture was concentrated under reduced pressure to give Compound 918n (170 mg, 257.80 μmol 98.16% purity) as a light yellow oil.

LCMS [M+1]⁺=623.6

¹H NMR (400 MHz, CHLOROFORM-d) δ=11.94-11.71 (m, 1H), 5.45-5.22 (m, 2H), 4.30-4.15 (m, 4H), 3.82-3.57 (m, 4H), 3.55-3.45 (m, 1H), 3.43-3.17 (m, 3H), 3.03-2.89 (m, 1H), 2.86-2.74 (m, 4H), 2.40-2.28 (m, 3H), 2.26-2.14 (m, 1H), 2.09-1.97 (m, 4H), 1.68-1.62 (m, 2H), 1.59-1.52 (m, 2H), 1.49-1.34 (m, 4H), 1.26 (br s, 20H), 1.20 (d, J=6.4 Hz, 3H), 1.13 (d, J=6.4 Hz, 3H), 0.96 (t, J=7.6 Hz, 3H), 0.88 (t, J=6.8 Hz, 6H)

### General Procedure for Preparation of Compound 918o:

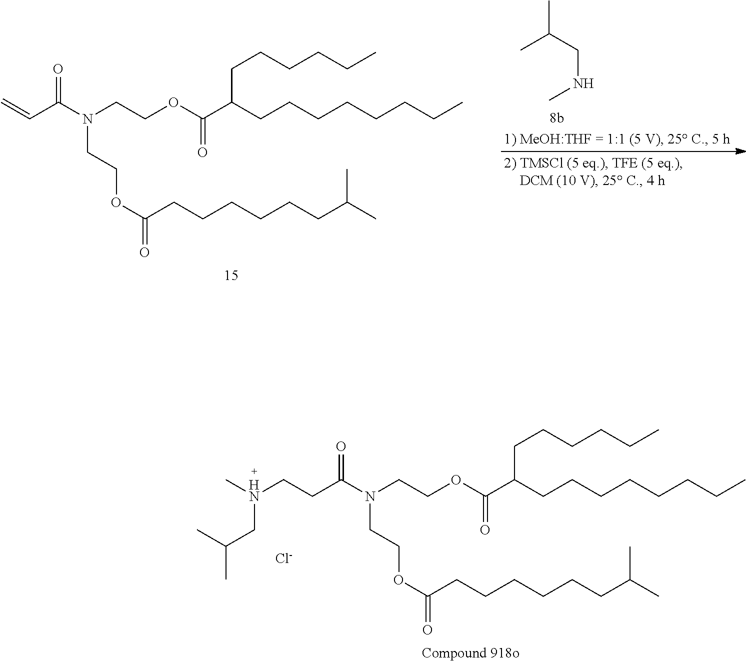

To a solution of Compound 15 (800 mg, 1.45 mmol, 1 eq.) in MeOH (2 mL) and THF (2 mL) was added Compound 8b (379.08 mg, 4.35 mmol, 3 eq.) at 25° C. The mixture was stirred at 25° C. for 2 hours. TLC (DCM:MeOH=10:1, R_{f}=0.4) indicated Compound 15 was consumed completely and many new spots formed. The reaction mixture was concentrated under reduced pressure to give a residue. The residue was purified by column chromatography (SiO₂, Petroleum ether:Ethyl acetate=80:20 to 0:1) to give Compound 918o (800 mg, 1.23 mmol, 78.78% yield, 98.48% purity, Free) as a light yellow oil.

LCMS [M+1]⁺=639.6

¹H NMR (400 MHz, CHLOROFORM-d) δ=4.26-4.17 (m, 4H), 3.69-3.56 (m, 4H), 2.77-2.64 (m, 2H), 2.56 (br s, 2H), 2.37-2.28 (m, 3H), 2.23 (br s, 3H), 2.15-2.07 (m, 2H), 1.80-1.69 (m, 1H), 1.65-1.62 (m, 1H), 1.60-1.58 (m, 2H), 1.52 (br dd, J=6.8, 13.2 Hz, 2H), 1.45 (br dd, J=5.2, 8.4 Hz, 2H), 1.32-1.21 (m, 26H), 1.18-1.11 (m, 2H), 0.98-0.79 (m, 18H).

To a solution of TFE (626.23 mg, 6.26 mmol, 450.20 μL, 5 eq.) and TMSCI (680.07 mg, 6.26 mmol, 794.48 μL, 5 eq.) in DCM (8 mL) was added Compound 918o (800 mg, 1.25 mmol, 1 eq.). The mixture was stirred at 20° C. for 3 hours. The reaction mixture was concentrated under reduced pressure to give Compound 918o (247 mg, 364.98 μ mol, 29.15% yield, 99.27% purity, 0.9 HCl) as a light yellow oil.

LCMS [M+1]⁺=639.6

¹H NMR (400 MHz, CHLOROFORM-d) δ=11.91 (br s, 1H), 4.32-4.09 (m, 4H), 3.78-3.56 (m, 4H), 3.49 (br s, 1H), 3.42-3.33 (m, 1H), 3.28 (br s, 2H), 2.99-2.87 (m, 1H), 2.86-2.72 (m, 4H), 2.38-2.27 (m, 3H), 2.26-2.11 (m, 1H), 1.63-1.57 (m, 3H), 1.52 (td, J=6.8, 13.6 Hz, 2H), 1.48-1.39 (m, 2H), 1.35-1.21 (m, 26H), 1.21-1.10 (m, 8H), 0.98-0.75 (m, 12H)

### General Procedure for Preparation of Compound 918p:

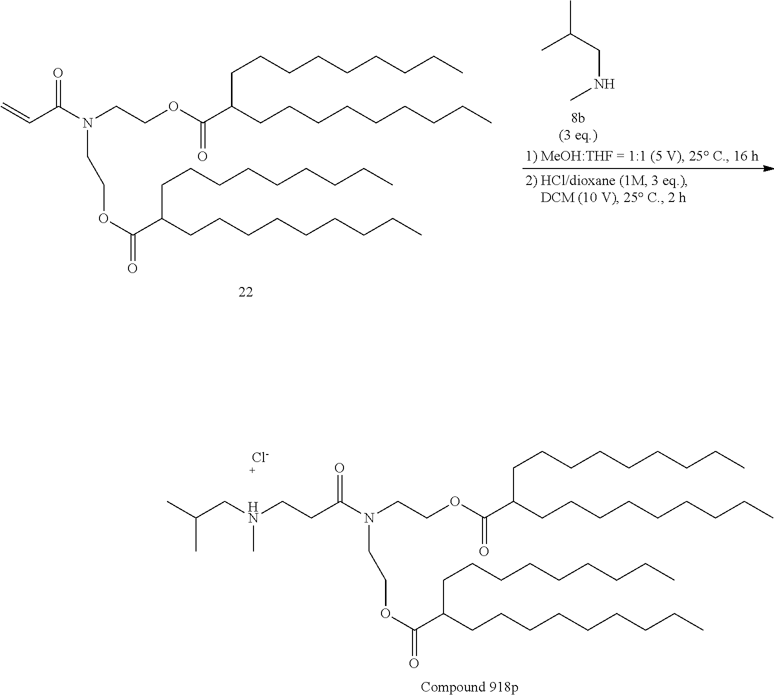

To a solution of Compound 22 (500 mg, 668.26 μmol, 1 eq.) in MeOH (1.25 mL) and THF (1.25 mL) was added Compound 8b (174.74 mg, 2.00 mmol, 3 eq.). The mixture was stirred at 25° C. for 16 hours. LCMS showed Compound 22 was consumed completely and one main peak with desired MS was detected. The reaction mixture was concentrated under reduced pressure to give a residue. The residue was purified by column chromatography (SiO₂, Petroleum ether:Ethyl acetate=20:1 to 5:1) to give Compound 918p (400 mg, 473.85 μmol, 79.17% yield, 98.96% purity) as a colorless oil.

LCMS [M+1]⁺=835.8

¹H NMR (400 MHz, CHLOROFORM-d) δ=4.21 (t, J=5.6 Hz, 4H), 3.63 (td, J=6.0, 14.4 Hz, 4H), 2.72 (br s, 2H), 2.57 (br s, 2H), 2.32 (dt, J=3.6, 8.8 Hz, 2H), 2.24 (br s, 3H), 2.19-2.04 (m, 2H), 1.81-1.71 (m, 1H), 1.61-1.52 (m, 4H), 1.44 (dt, J=5.2, 8.8 Hz, 4H), 1.32-1.20 (m, 56H), 0.99-0.76 (m, 18H).

To a solution of Compound 918p\400 mg, 478.83 μmol, 1 eq.) in DCM (4 mL) was added HCl/dioxane (1 M, 1.44 mL, 3 eq.). The mixture was stirred at 25° C. for 2 hours. The reaction mixture was concentrated and lyophilized to give Compound 918p (350 mg, 400.83 μmol, 83.71% yield, 99.01% purity, 0.8 HCl) as a colorless gum.

LCMS [M+1]⁺=835.8

¹H NMR (400 MHz, CHLOROFORM-d) δ=11.76 (br s, 1H), 4.24 (br s, 2H), 4.19 (t, J=5.6 Hz, 2H), 3.80-3.56 (m, 4H), 3.50 (br s, 1H), 3.36 (br d, J=6.0 Hz, 1H), 3.26 (br s, 2H), 2.95 (ddd, J=4.4, 7.6, 12.0 Hz, 1H), 2.87-2.72 (m, 4H), 2.40-2.27 (m, 2H), 2.19 (td, J=6.4, 13.2 Hz, 1H), 1.62-1.51 (m, 4H), 1.45 (br dd, J=5.2, 9.2 Hz, 4H), 1.25 (s, 56H), 1.19 (d, J=6.4 Hz, 3H), 1.13 (d, J=6.8 Hz, 3H), 0.93-0.83 (m, 12H)

### General Procedure for Preparation of Compounds 951-954:

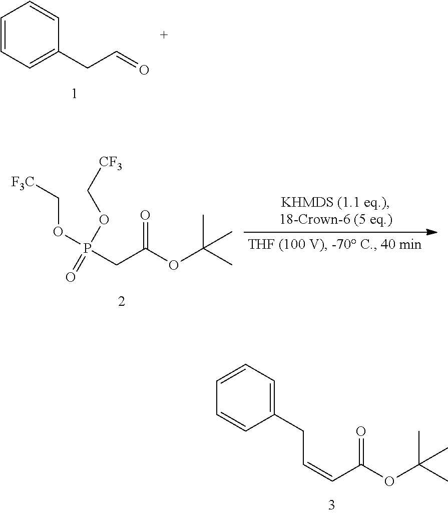 [SMILES: O=CCc1ccccc1 ; CC(C)(C)OC(=O)CP(=O)(OCC(F)(F)F)OCC(F)(F)F ; CC(C)(C)OC(=O)/C=C\Cc1ccccc1]

The reaction was set up for 2 batches in parallel. To a solution of Compound 2 (46.17 g, 128.18 mmol, 1.1 eq.) and 1,4,7,10,13,16-hexaoxacyclooctadecane (154.00 g, 582.61 mmol, 5 eq.) in THE (1500 mL) was added KHMDS (1 M, 128.18 mL, 1.1 eq.) dropwise at −70° C. The mixture was stirred at −70° C. for 10 min. Compound 1 (14 g, 116.52 mmol, 9.09 mL, 1 eq.) was added dropwise at −70° C. The mixture was stirred at −70° C. for 30 min. TLC (Petroleum ether:Ethyl acetate=10:1, R_{f}=0.6) showed Compound 1 was consumed completely. Two reactions were combined for workup. The reaction mixture was quenched by addition NH₄Cl (2000 mL) and extracted with EtOAc (2000 mL×2). The combined organic layers were washed with brine (3000 mL), dried over Na₂SO₄, filtered and concentrated under reduced pressure to give a residue. The residue was purified by column chromatography (SiO₂, Petroleum ether/Ethyl acetate=100/1 to 10/1) to give Compound 3 (25 g, 114.53 mmol, 49.14% yield) as a yellow oil.

¹H NMR (400 MHz, CHLOROFORM-d) δ=7.34-7.28 (m, 2H), 7.26-7.20 (m, 3H), 6.26 (td, J=7.6, 11.6 Hz, 1H), 5.78 (td, J=1.6, 11.6 Hz, 1H), 4.00 (dd, J=1.2, 7.6 Hz, 2H), 1.52 (s, 9H).

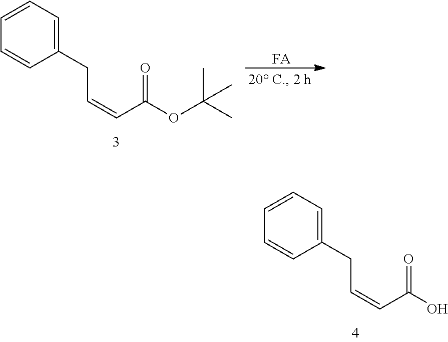

A mixture of Compound 3 (10 g, 45.81 mmol, 1 eq.) in formic acid (50 mL) was stirred at 20° C. for 2 hours under an N₂ atmosphere. TLC (Petroleum ether:Ethyl acetate=10:1, R_{f}=0) showed Compound 3 was consumed completely. Water was added to the reaction mixture (150 mL), and the solution was extracted with EtOAc (100 mL×3). The combined organic layers were washed with brine (100 mL×3), dried over Na₂SO₄, filtered and concentrated under reduced pressure to give a residue. The residue was dissolved in H₂O (50 mL) and ACN (10 mL), and the mixture was lyophilized to give Compound 4 (7.3 g, 45.01 mmol, 98.25% yield) as a white solid.

¹H NMR (400 MHz, CHLOROFORM-d) δ=13.10-10.40 (m, 1H), 7.37-7.30 (m, 2H), 7.27-7.21 (m, 3H), 6.52 (td, J=7.6, 11.6 Hz, 1H), 5.92 (td, J=1.6, 11.6 Hz, 1H), 4.06 (dd, J=1.2, 7.6 Hz, 2H)

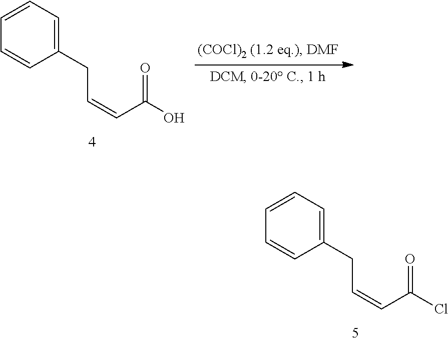 [SMILES: O=C(O)/C=C\Cc1ccccc1 ; O=C(Cl)/C=C\Cc1ccccc1]

To a solution of Compound 4 (2.4 g, 14.80 mmol, 1 eq.) in DCM (20 mL) was added DMF (54.08 mg, 739.90 μmol, 56.93 μL, 0.05 eq.) and (COCl)₂ (2.25 g, 17.76 mmol, 1.55 mL, 1.2 eq.) at 0° C. The mixture was stirred at 20° C. for 1 hour. TLC (Petroleum ether:Ethyl acetate=3:1, R_{f}=0.8, quenched by MeOH) indicated that Compound 4 was consumed completely and one new spot formed. The reaction mixture was concentrated under reduced pressure to give Compound 5 (2.67 g, crude) as a yellow oil. The crude product was used in the next step without further purification.

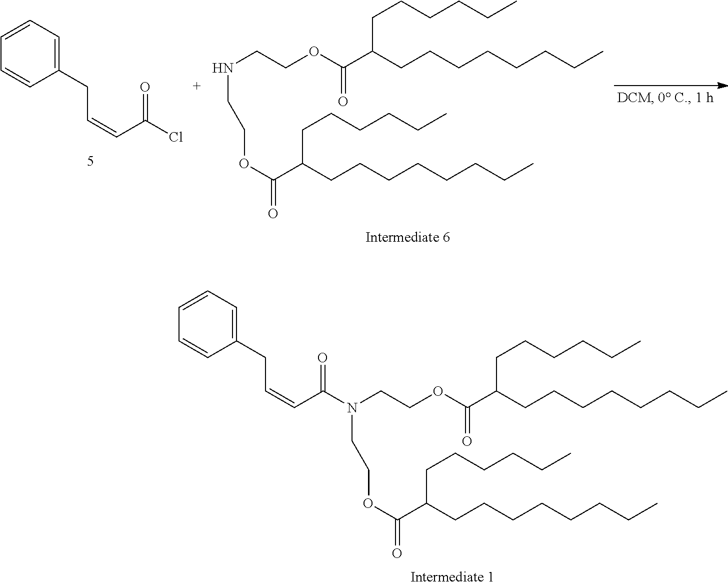 [SMILES: O=C(Cl)/C=C\Cc1ccccc1 ; CCCCCCCCC(CCCCCC)C(=O)OCCNCCOC(=O)C(CCCCCC)CCCCCCCC ; CCCCCCCCC(CCCCCC)C(=O)OCCN(CCOC(=O)C(CCCCCC)CCCCCCCC)C(=O)/C=C\Cc1ccccc1]

To a solution of Compound 5 (2.61 g, 14.43 mmol, 1.4 eq.) in DCM (60 mL) was added Intermediate 6 (6 g, 10.31 mmol, 1 eq.) in DCM (10 mL) dropwise at 0° C. The mixture was stirred at 0° C. for 1 hour. TLC (Petroleum ether:Ethyl acetate=3:1, R_{f}=0.7) indicated Intermediate 6 was consumed completely and one major new spot formed. The mixture was added to water (5 mL) and extracted with DCM (5 mL×2). The combined organic layers were washed with brine (5 mL), dried over Na₂SO₄, filtered and concentrated under reduced pressure to give a residue. The residue was purified by column chromatography (SiO₂, Petroleum ether/Ethyl acetate=100/1 to 1/1) to give Intermediate 1 (4.3 g, 5.84 mmol, 56.66% yield, 98.65% purity) as a colorless oil.

The resulting product was dissolved in DCM (10 mL) and filtered to remove the insoluble material. The filter liquor was concentrated in vacuo; Intermediate 1 (5.83 g, 7.93 mmol, 98.80% yield, 98.79% purity) is a light yellow oil.

LCMS [M+1]⁺=726.6

¹H NMR (400 MHz, METHANOL-d4) δ=7.30-7.18 (m, 5H), 6.29 (d, J=11.6 Hz, 1H), 6.19-6.11 (m, 1H), 4.27 (td, J=5.6, 19.6 Hz, 4H), 3.76 (td, J=5.6, 13.6 Hz, 4H), 3.69 (d, J=7.6 Hz, 2H), 2.41-2.32 (m, 2H), 1.58 (br dd, J=5.6, 7.2 Hz, 4H), 1.49-1.41 (m, 4H), 1.27 (br s, 40H), 0.92-0.86 (m, 12H)

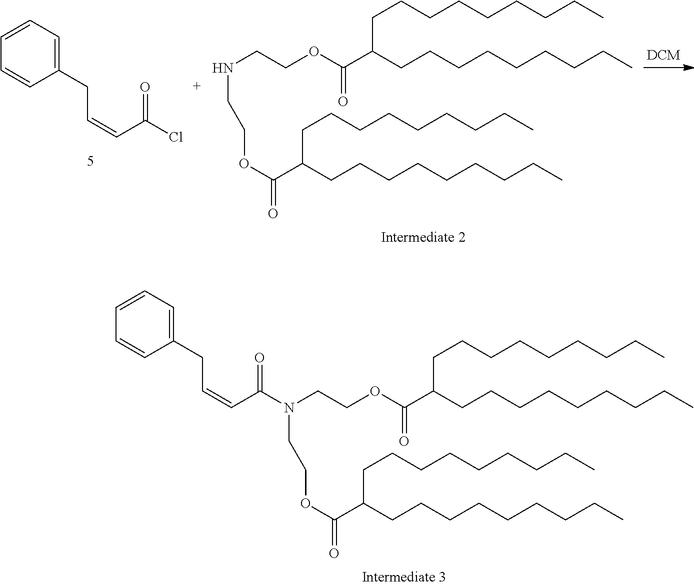 [SMILES: O=C(Cl)/C=C\Cc1ccccc1 ; CCCCCCCCCC(CCCCCCCCC)C(=O)OCCNCCOC(=O)C(CCCCCCCCC)CCCCCCCCC ; CCCCCCCCCC(CCCCCCCCC)C(=O)OCCN(CCOC(=O)C(CCCCCCCCC)CCCCCCCCC)C(=O)/C=C\Cc1ccccc1]

To a solution of Intermediate 2 (4 g, 5.76 mmol, 1 eq.) in DCM (40 mL) was added Compound 5 (1.25 g, 6.91 mmol, 1.2 eq.) at 0° C. The mixture was stirred at 0° C. for 1 hour. TLC (Petroleum ether:Ethyl acetate=3:1, R_{f}=0.36) indicated Intermediate 2 was consumed completely, and one major new spot with greater polarity was detected. The reaction mixture was diluted with H₂O (50 mL) and extracted with DCM (50 mL×3), dried over Na₂SO₄, filtered and concentrated under reduced pressure to give a residue. The residue was purified by column chromatography (SiO₂, Petroleum ether/Ethyl acetate=100/1 to 95/5) to give Intermediate 3 (3.94 g, 4.67 mmol, 81.11% yield, 99.45% purity) as a colorless oil.

LCMS [M+1]⁺=838.7

¹H NMR (400 MHz, METHANOL-d4) δ=7.42-7.04 (m, 5H), 6.32-6.24 (m, 1H), 6.20-6.10 (m, 1H), 4.30 (t, J=5.6 Hz, 2H), 4.25 (t, J=5.8 Hz, 2H), 3.81-3.72 (m, 4H), 3.69 (d, J=7.6 Hz, 2H), 2.40-2.31 (m, 2H), 1.64-1.54 (m, 4H), 1.49-1.41 (m, 4H), 1.28 (br s, 56H), 0.89 (t, J=6.8 Hz, 12H)

### General Procedure for Preparation of Compound 951:

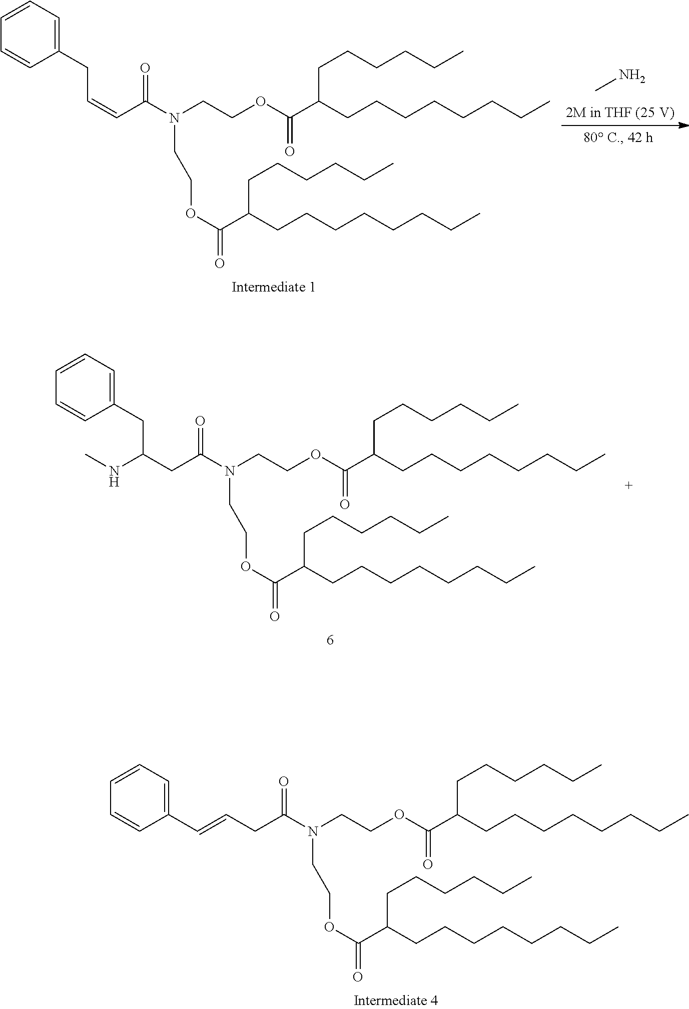

A mixture of Intermediate 1 (700 mg, 964.03 μmol, 1 eq.) in methanamine (2 M, 17.50 mL, 36.31 eq.) and the mixture was stirred at 80° C. for 2 hours in a 30-ml, sealed tube at 15 Psi. Then, DBU (293.52 mg, 1.93 mmol, 290.61 μL, 2 eq.) was added at 25° C., and the mixture was stirred at 80° C. for 40 hours in a 30-mL of sealed tube at 15 Psi. LCMS indicated Intermediate 1 remained, and one peak with desired MS was detected. The reaction mixture was concentrated under reduced pressure to give a residue. The residue was purified by column chromatography (SiO₂, Dichloromethane:Methanol=100:1 to 1:1) to give crude Compound 6 (200 mg) as a colorless oil and Intermediate 4 (879 mg, 1.19 mmol, 61.69% yield, 98.25% purity) as a colorless oil. The crude Compound 6 (200 mg) was re-purified by column chromatography (SiO₂, Dichloromethane:Methanol=100:1 to 1:1) to give (80 mg, 100.15 μmol, 5.19% yield, 94.79% purity) as a colorless oil.

LCMS [M+1]⁺=757.6

1H NMR (400 MHz, CHLOROFORM-d) δ=7.29-7.20 (m, 2H), 7.17-7.09 (m, 3H), 4.13 (t, J=5.6 Hz, 2H), 4.07-3.91 (m, 2H), 3.58-3.48 (m, 2H), 3.48-3.30 (m, 2H), 3.21-3.06 (m, 1H), 2.87-2.76 (m, 1H), 2.67 (dd, J=7.2, 13.6 Hz, 1H), 2.46-2.35 (m, 5H), 2.32-2.09 (m, 2H), 1.49-1.41 (m, 4H), 1.40-1.29 (m, 4H), 1.18 (br s, 40H), 0.81 (br t, J=6.4 Hz, 12H)

LCMS [M+1]⁺=726.6

¹H NMR (400 MHz, CHLOROFORM-d) δ=7.34-7.27 (m, 2H), 7.22 (t, J=7.6 Hz, 2H), 7.18-7.12 (m, 1H), 6.45-6.37 (m, 1H), 6.34-6.23 (m, 1H), 4.23-4.10 (m, 4H), 3.64-3.47 (m, 4H), 3.34-3.26 (m, 2H), 2.26 (dtd, J=3.2, 5.6, 14.4 Hz, 2H), 1.51 (br d, J=7.2 Hz, 4H), 1.44-1.33 (m, 4H), 1.17 (br s, 40H), 0.85-0.73 (m, 12H)

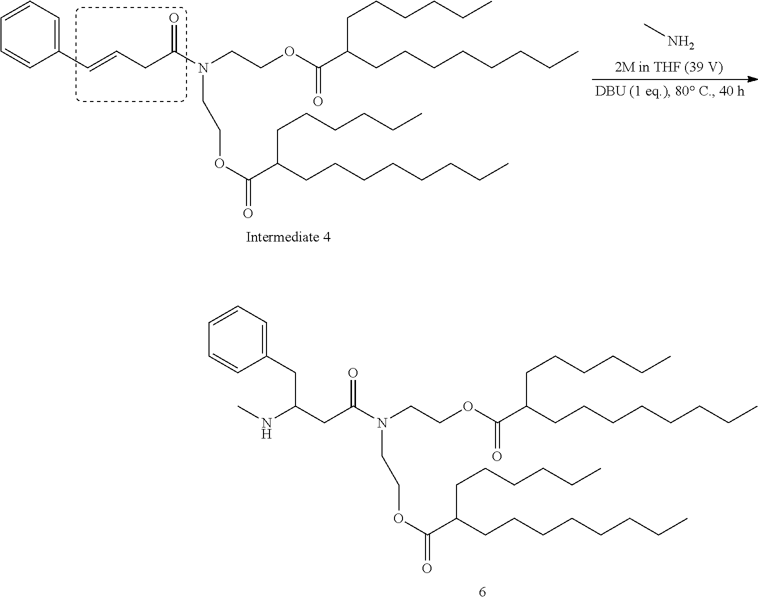

A mixture of Intermediate 4 (640 mg, 881.40 μmol, 1 eq.) and DBU (134.18 mg, 881.40 μmol, 132.86 μL, 1 eq.) was prepared in methanamine (2 M, 25 mL, 56.73 eq.) at 25° C. and the mixture was stirred at 80° C. for 42 hours in a 30-mL sealed tube at 15 Psi. LCMS indicated Intermediate 4 remained, and one peak with desired MS was detected. The reaction mixture was concentrated under reduced pressure to give a residue. The residue was purified by flash silica gel chromatography (ISCO®; 40 g SepaFlash® Silica Flash Column, 0-50% Ethyl acetate/Petroleum ether gradient @80 mL/min) to give crude Compound 6 (340 mg, 416.53 μmol, 23.63% yield, 92.76% purity) as a colorless oil. The crude Compound 6 (340 mg) was re-purified by prep-HPLC (column: X-Select CSH Phenyl-Hexyl 100× 30 5u; mobile phase: [H₂O (0.04% HCl)-ACN:THF=1:1]; gradient: 50%-90% B over 15.0 min) to give Compound 6 (320 mg, 401.07 μmol, 72.30% yield, 99.47% purity, HCl) as a colorless gum. The Compound 6 (310 mg, 390.61 μmol, 1 eq, HCl) was dissolved in ethyl acetate (20 mL) and then basified with saturated NaHCO₃ to pH=7. The reaction mixture was extracted with ethyl acetate (10 mL×2). The combined organic layers were dried over Na₂SO₄, filtered and concentrated under reduced pressure to give Compound 6 (275 mg, 354.40 μmol, 90.73% yield, 97.58% purity) as a colorless oil.

LCMS [M+1]⁺=757.1

¹H NMR (400 MHz, CHLOROFORM-d) δ=7.26-7.20 (m, 2H), 7.17-7.11 (m, 3H), 4.13 (t, J=6.0 Hz, 2H), 4.07-3.90 (m, 2H), 3.52 (t, J=6.0 Hz, 2H), 3.48-3.33 (m, 2H), 3.13 (quin, J=6.4 Hz, 1H), 2.84-2.74 (m, 1H), 2.71-2.60 (m, 1H), 2.48-2.28 (m, 5H), 2.26-2.11 (m, 2H), 1.47-1.43 (m, 4H), 1.40-1.32 (m, 4H), 1.18 (br s, 40H), 0.85-0.74 (m, 12H).

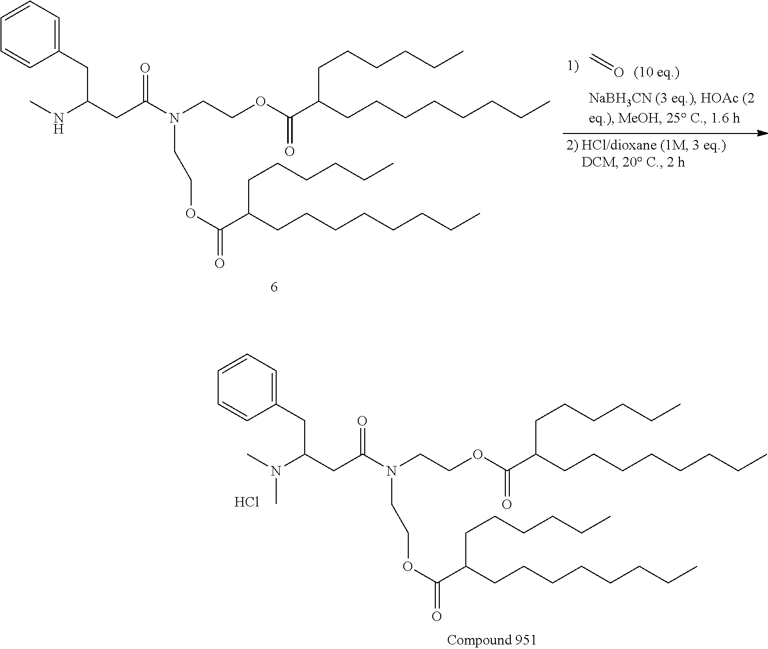

To a solution of Compound 6 (275 mg, 363.19 μmol, 1 eq.) in MEOH (3 mL) was added formaldehyde (294.73 mg, 3.63 mmol, 270.40 μL, 10 eq.) and HOAc (43.62 mg, 3.63 mmol, 1 eq.) at 25° C.; the mixture was stirred at 25° C. for 6 min. Next was added NaBH₃CN (68.47 mg, 726.38 μmol, 3 eq) at 0° C. The mixture was stirred at 25° C. for 1.5 hours. TLC (Dichloromethane:Methanol=10:1, R_{f}=0.58) indicated Compound 6 was consumed completely, and one major new spot was detected. The reaction mixture was diluted with Sat.NaHCO₃ (40 mL) and extracted with DCM (30 mL×2). The combined organic layers were dried over Na₂SO₄, filtered and concentrated under reduced pressure to give a residue. The residue was purified by flash silica gel chromatography (ISCO®; 4 g SepaFlash® Silica Flash Column, 0-50% Ethylacetate/Petroleum ether gradient @50 mL/min) to give Compound 951 (130 mg, 166.92 μmol, 45.96% yield, 99.02% purity) as a colorless oil.

LCMS [M+1]⁺=771.7

¹H NMR (400 MHz, CHLOROFORM-d) δ=7.28-7.17 (m, 5H), 4.24-4.07 (m, 4H), 3.68-3.46 (m, 5H), 3.07-2.86 (m, 1H), 2.75-2.53 (m, 2H), 2.53-2.34 (m, 6H), 2.34-2.20 (m, 3H), 1.57-1.53 (m, 4H), 1.50-1.42 (m, 4H), 1.27 (br s, 40H), 0.90 (br t, J=6.8 Hz, 12H)

To a solution of Compound 951 (130 mg, 168.57 μmol, 1 eq.) in DCM (1.3 mL) was added HCl/dioxane (0.5 M, 505.70 μL, 1.5 eq.) at 25° C. The mixture was stirred at 25° C. for 2 hours. The reaction mixture was concentrated and lyophilized to give Compound 951 (103.2 mg, 127.75 μmol, 75.79% yield, 99.08% purity, 0.8 HCl) as a colorless gum.

LCMS [M+1]⁺=771.7

¹H NMR (400 MHz, CHLOROFORM-d) δ=13.06-11.63 (m, 1H), 7.29-7.20 (m, 5H), 4.25-3.93 (m, 5H), 3.63-3.50 (m, 1H), 3.50-3.42 (m, 2H), 3.42-3.30 (m, 1H), 3.29-3.08 (m, 2H), 3.06-2.91 (m, 1H), 2.85-2.77 (m, 1H), 2.87-2.46 (m, 7H), 2.27-2.19 (m, 1H), 2.18-2.07 (m, 1H), 1.49-1.41 (m, 4H), 1.40-1.30 (m, 4H), 1.18 (br s, 40H), 0.81 (br t, J=6.6 Hz, 12H).

### General Procedure for Preparation of Compound 953

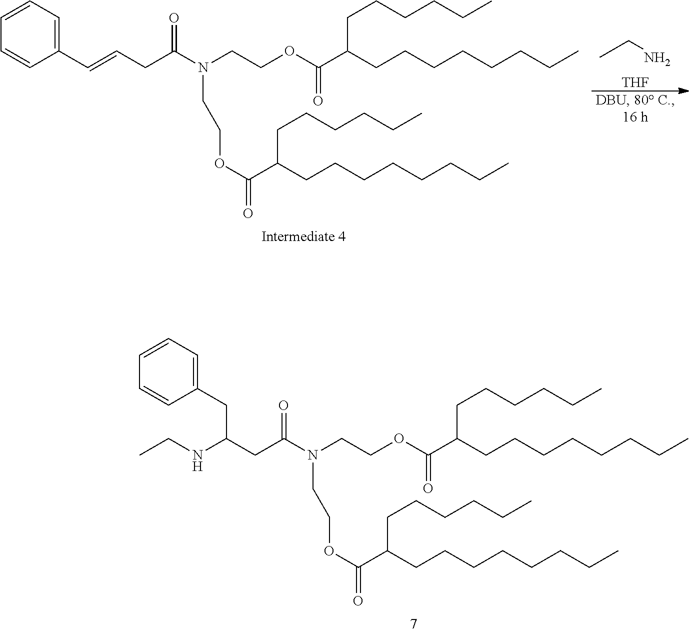

A solution of Intermediate 4 (300 mg, 413.15 μmol, 1 eq.) and DBU (62.90 mg, 413.15 μmol, 62.27 μL, 1 eq.) in ethanamine (2 M, 25 mL, 121.02 eq.) was stirred at 80° C. for 40 hours in a 30-mL sealed tube at 15 Psi. LCMS showed Intermediate 4 remained, and the desired MS was detected. The reaction mixture or solution was concentrated to give a residue. The residue was purified by column chromatography (SiO₂, DCM:MeOH=99:1 to 96:4) to give Compound 7 (169 mg, 212.34 μmol, 51.40% yield, 96.9% purity) as a light yellow oil.

LCMS [M+1]⁺=771.8

¹H NMR (400 MHz, CHLOROFORM-d) δ=7.33-7.28 (m, 2H), 7.24-7.18 (m, 3H), 4.20 (t, J=5.6 Hz, 2H), 4.14-3.99 (m, 2H), 3.60 (t, J=6.0 Hz, 2H), 3.57-3.39 (m, 2H), 3.31 (quin, J=6.0 Hz, 1H), 2.93-2.82 (m, 1H), 2.79-2.62 (m, 3H), 2.44 (br d, J=5.6 Hz, 2H), 2.36-2.20 (m, 2H), 1.57-1.50 (m, 4H), 1.43 (br dd, J=5.6, 13.6 Hz, 4H), 1.25 (br s, 40H), 1.08 (t, J=7.2 Hz, 3H), 0.89 (br t, J=6.8 Hz, 12H)

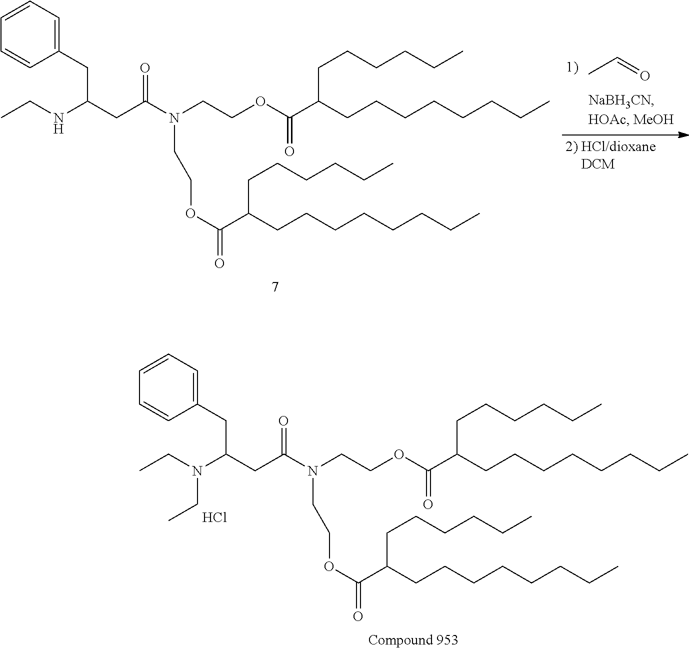

To a solution of Compound 7 (264 mg, 342.32 μmol, 1 eq.) and acetaldehyde (377.00 mg, 3.42 mmol, 480.26 μL, 10 eq.) in MeOH (10 mL) was added HOAc (205.57 mg, 3.42 mmol, 195.97 μL, 10 eq.) and NaBH₃CN (64.54 mg, 1.03 mmol, 3.00 eq.). The mixture was stirred at 25° C. for 3 hours. TLC (DCM:MeOH=15:1, R_{f}=0.48) indicated Compound 2 was consumed completely and one new spot formed. The reaction mixture was partitioned between EtOAc (50 mL) and saturated NaHCO₃ (50 mL). The organic layer was separated and concentrated under reduced pressure to give a residue. The residue was purified by column chromatography (SiO₂, DCM:MeOH=99:1 to 97:3) to give Compound 953 (190 mg, 235.34 μmol, 68.75% yield, 99.0% purity) as a yellow oil.

LCMS [M+1]⁺=799.6

¹H NMR (400 MHz, CHLOROFORM-d) δ=7.27-7.13 (m, 5H), 4.22-4.06 (m, 4H), 3.70-3.42 (m, 5H), 2.92-2.80 (m, 1H), 2.62-2.45 (m, 6H), 2.38-2.19 (m, 3H), 1.58 (br s, 4H), 1.48-1.40 (m, 4H), 1.26 (br s, 40H), 1.06-0.94 (m, 6H), 0.89 (t, J=6.0 Hz, 12H)

To a solution of Compound 953 (160 mg, 200.19 μmol, 1 eq.) in DCM (1.6 mL) was added HCl/dioxane (0.5 M, 600.56 μL, 1.5 eq.) at 25° C. The mixture was stirred at 25° C. for 2 hours. The reaction mixture was concentrated and lyophilized to give Compound 953 (102 mg, 121.44 μmol, 60.67% yield, 98.2% purity, 0.7 HCl) as a colorless oil.

LCMS [M+1]⁺=799.6

¹H NMR (400 MHz, CHLOROFORM-d) δ=11.80-11.49 (m, 1H), 7.37-7.27 (m, 5H), 4.49-4.27 (m, 1H), 4.24-3.93 (m, 4H), 3.74-3.60 (m, 1H), 3.58-3.26 (m, 6H), 3.25-2.97 (m, 4H), 2.80 (br dd, J=5.6, 17.2 Hz, 1H), 2.39-2.09 (m, 2H), 1.59-1.36 (m, 14H), 1.26 (br s, 40H), 0.88 (br t, J=6.8 Hz, 12H).

### General Procedure for Preparation of Compound 952

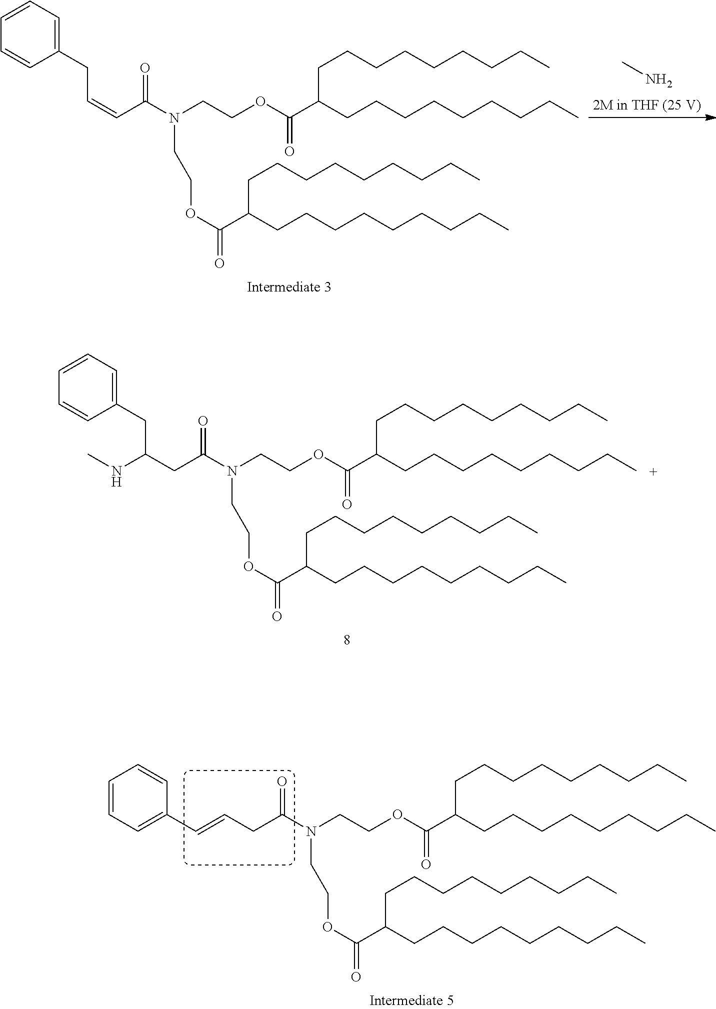

A mixture of Intermediate 3 (1 g, 1.19 mmol, 1 eq.) in methanamine (2 M, 25 mL, 41.92 eq.) was stirred at 80° C. for 2 hours in a 30-mL sealed tube; DBU (363.19 mg, 2.39 mmol, 359.59 μL, 2 eq.) was added at 25° C. and the mixture was stirred at 80° C. for 52 hours in a 30-mL sealed tube. LCMS showed that Intermediate 3 remained and the desired MS was detected. The reaction mixture was concentrated under reduced pressure to give a residue. The residue was purified by column chromatography (SiO₂, Dichloromethane:Methanol=100:0 to 1:1) to give crude product (240 mg). The crude product (240 mg) was re-purified by prep-HPLC (column: X-Select CSH Phenyl-Hexyl 100×30 5u; mobile phase: [H2O (0.04% HCl)-ACN:THF=1:1]; gradient: 40%-90% B over 14.0 min) to give Compound 8 (180 mg, 194.36 μmol, 8.15% yield, 97.81% purity, HCl) as a yellow oil, and Intermediate 5 (1.1 g, 1.27 mmol, 53.27% yield, 96.85% purity) as a yellow oil.

LCMS [M+1]⁺=869.8

¹H NMR (400 MHz, CHLOROFORM-d) δ=10.95-10.48 (m, 1H), 9.17-8.92 (m, 1H), 7.30-7.20 (m, 3H), 7.19-7.13 (m, 2H), 4.12 (br t, J=5.6 Hz, 2H), 4.06-3.95 (m, 1H), 3.83 (td, J=5.6, 11.6 Hz, 1H), 3.59-3.37 (m, 5H), 3.28 (td, J=5.2, 15.6 Hz, 1H), 3.05 (br dd, J=11.2, 13.2 Hz, 1H), 2.98-2.85 (m, 1H), 2.79 (br s, 1H), 2.74 (br t, J=4.8 Hz, 3H), 2.29-2.19 (m, 1H), 2.09-1.96 (m, 1H), 1.55-1.42 (m, 4H), 1.42-1.32 (m, 4H), 1.18 (br s, 56H), 0.87-0.74 (m, 12H)

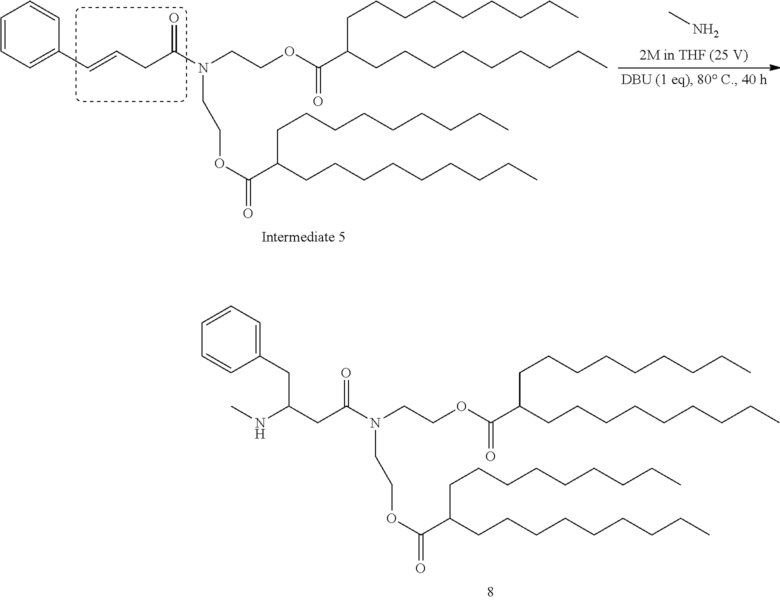

To a solution of Intermediate 5 (1.00 g, 1.19 mmol, 1 eq.) in methanamine (2 M, 25 mL, 41.92 eq.) was added DBU (181.60 mg, 1.19 mmol, 179.80 L, 1 eq.) at 25° C., and the mixture was stirred at 80° C. for 40 hours in a 30-mL sealed tube at 15 Psi. TLC (Dichloromethane:Methanol=10:1, R_{f}=0.5) indicated Intermediate 5 remained, and one major new spot was detected. The reaction mixture was concentrated under reduced pressure to give a residue.

The residue was purified by column chromatography (SiO₂, Dichloromethane:Methanol=100:0 to 1:1) to give Compound 8 (310 mg, 346.66 μmol, 29.06% yield, 97.22% purity) as a yellow oil.

LCMS [M+1]⁺=869.8

¹H NMR (400 MHz, CHLOROFORM-d) δ=7.26-7.20 (m, 2H), 7.18-7.10 (m, 3H), 4.16-4.07 (m, 2H), 4.06-3.91 (m, 2H), 3.52 (br t, J=5.6 Hz, 2H), 3.47-3.32 (m, 2H), 3.17 (quin, J=6.4 Hz, 1H), 2.91-2.83 (m, 1H), 2.83-2.63 (m, 2H), 2.49-2.33 (m, 5H), 2.32-2.10 (m, 2H), 1.53-1.42 (m, 4H), 1.39-1.29 (m, 4H), 1.23-1.10 (m, 56H), 0.81 (t, J=6.8 Hz, 12H).

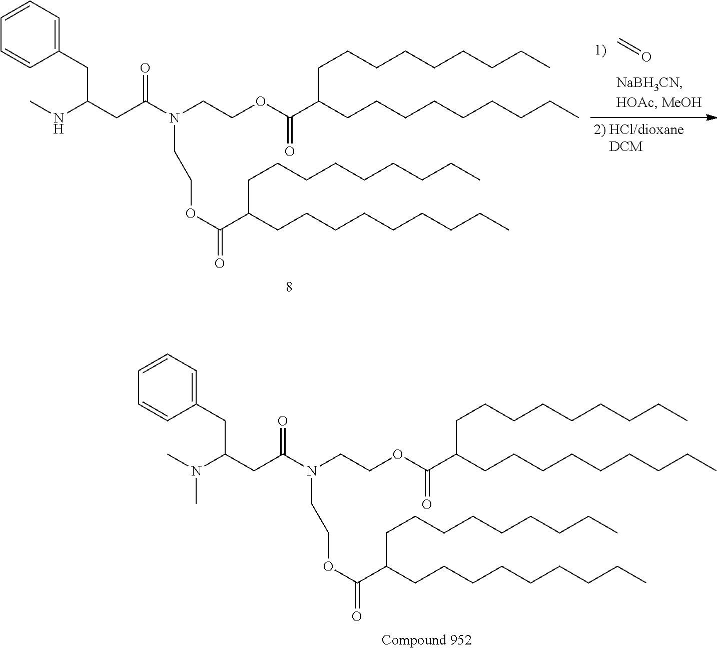

To a solution of Compound 8 (310 mg, 356.57 μmol, 1 eq.) in MeOH (3 mL) was added AcOH (21.41 mg, 356.57 μmol, 20.41 μL, 1 eq.), formaldehyde (289.36 mg, 3.57 mmol, 265.47 μL, 10 eq.) and NaBH₃CN (67.22 mg, 1.07 mmol, 3 eq.) at 0° C. The mixture was stirred at 25° C. for 2 hours. TLC (Dichloromethane:Methanol=10:1, R_{f}=0.55) indicated Compound 8 was consumed completely and one new spot formed. The reaction mixture was concentrated under reduced pressure to give a residue. The residue was purified by column chromatography (SiO₂, Dichloromethane:Methanol=100:0 to 1:1) to give a crude product (240 mg). The crude product (240 mg) was re-purified by prep-HPLC (column: Agela DuraShell NH₂ 150 mm×30 mm×5 um; mobile phase: [Heptane-THF:ACN=2:1]; gradient: 0%-30% B over 10.0 min) to give Compound 952 (100 mg, 112.42 μmol, 31.53% yield, 99.31% purity, free) as a colorless oil.

LCMS [M+1]⁺=883.8

¹H NMR (400 MHz, CHLOROFORM-d) δ=7.23-7.20 (m, 1H), 7.19-7.08 (m, 4H), 4.11-3.96 (m, 4H), 3.57-3.32 (m, 5H), 2.97-2.79 (m, 1H), 2.68-2.47 (m, 2H), 2.45-2.05 (m, 9H), 1.49-1.42 (m, 4H), 1.38-1.31 (m, 4H), 1.17 (br s, 56H), 0.81 (t, J=6.8 Hz, 12H)

To a solution of Compound 952 (100 mg, 113.20 μmol, 1 eq., free) in DCM (2 mL) was added HCl/dioxane (0.5 M, 339.59 μL, 1.5 eq.) at 25° C. The mixture was stirred at 25° C. for 2 hours. The reaction mixture was concentrated and lyophilized to give Compound 952 (102 mg, 99.91% purity, HCl).

LCMS [M+1]⁺=883.8

¹H NMR (400 MHz, CHLOROFORM-d) δ=7.28-7.20 (m, 4H), 7.19-7.15 (m, 1H), 4.14-4.00 (m, 4H), 4.00-3.92 (m, 1H), 3.63-3.54 (m, 1H), 3.53-3.40 (m, 2H), 3.34 (td, J=5.6, 15.2 Hz, 1H), 3.24-3.10 (m, 2H), 3.03-2.92 (m, 1H), 2.80-2.54 (m, 7H), 2.27-2.19 (m, 1H), 2.15-2.06 (m, 1H), 1.50-1.40 (m, 4H), 1.37-1.28 (m, 4H), 1.25-1.11 (m, 56H), 0.81 (t, J=6.8 Hz, 12H)

### General Procedure for Preparation of Compound 954

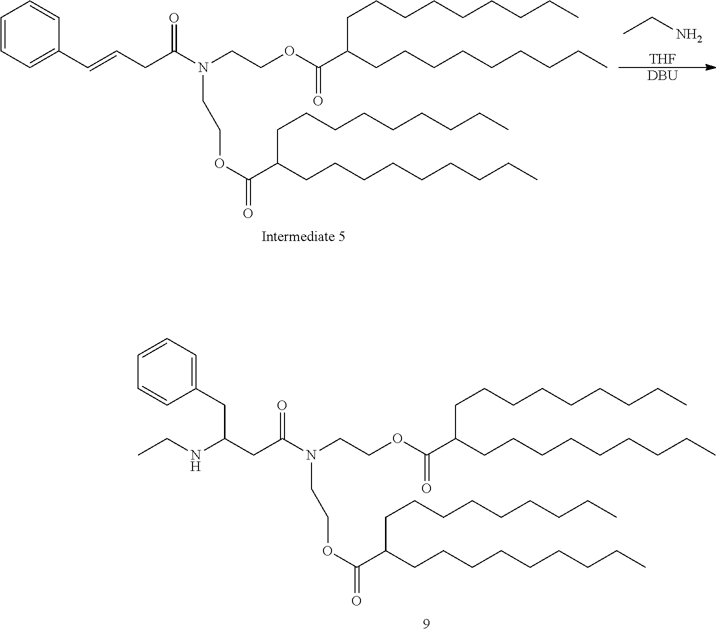

A mixture of Intermediate 5 (1.00 g, 1.19 mmol, 1 eq.) and DBU (181.60 mg, 1.19 mmol, 179.80 μL, 1 eq.) in ethanamine (2 M, 25.00 mL, 41.92 eq.) was degassed and purged with N₂ 3 times, and the mixture was stirred at 80° C. for 36 hours under an N₂ atmosphere. LCMS showed Intermediate 5 remained and the desired MS was detected. The reaction mixture was concentrated under reduced pressure to give a residue. The residue was purified by column chromatography (SiO₂, Dichloromethane:Methanol=50:1 to 40:1) to give Compound 9 (480 mg, 527.59 μmol, 44.23% yield, 97.1% purity) as a yellow oil.

LCMS [M+1]⁺=883.7

¹H NMR (400 MHz, CHLOROFORM-d) δ=7.33-7.27 (m, 2H), 7.25-7.18 (m, 3H), 4.20 (t, J=6.0 Hz, 2H), 4.14-4.01 (m, 2H), 3.60 (t, J=6.0 Hz, 2H), 3.55-3.37 (m, 2H), 3.31 (quin, J=6.4 Hz, 1H), 2.92-2.82 (m, 1H), 2.81-2.62 (m, 3H), 2.44 (br d, J=6.0 Hz, 2H), 2.36-2.21 (m, 2H), 1.60-1.51 (m, 4H), 1.47-1.37 (m, 4H), 1.33-1.20 (m, 56H), 1.08 (t, J=7.2 Hz, 3H), 0.89 (t, J=6.8 Hz, 12H).

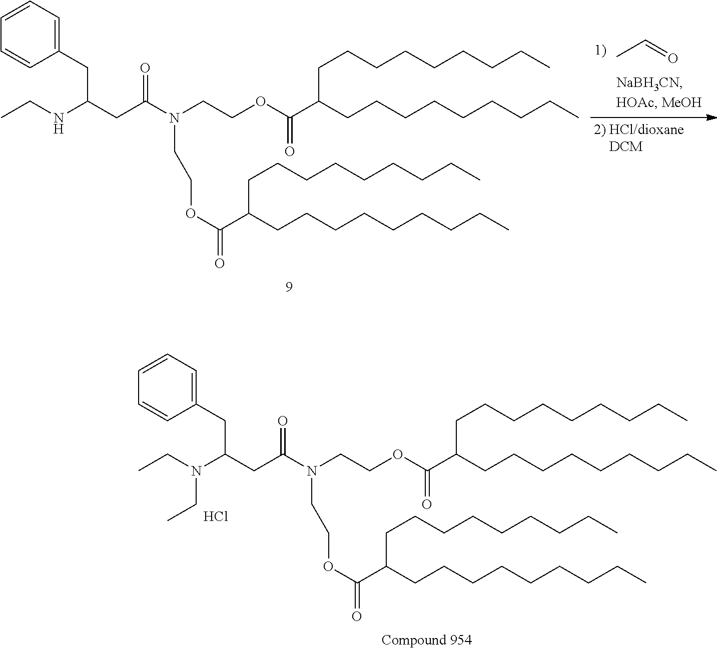

To a solution of Compound 9 (480 mg, 543.35 μmol, 1 eq.), acetaldehyde (598.39 mg, 5.43 mmol, 762.28 μL, 10 eq.) and HOAc (326.29 mg, 5.43 mmol, 311.05 μL, 10 eq.) in MeOH (20 mL) was added NaBH₃CN (102.43 mg, 1.63 mmol, 3 eq.) at 0° C. The resulting mixture was stirred at 25° C. for 3 hours. LCMS showed Compound 9 was consumed and the desired MS was detected. The reaction mixture was diluted with Sat.NaHCO₃ (20 mL) and extracted with ethyl acetate (15 mL×2). The combined organic layers were dried over Na₂SO₄, filtered and concentrated under reduced pressure to give a residue. The residue was purified by column chromatography (SiO₂, Dichloromethane:Methanol=100:1 to 80:1) to give crude product (350 mg), and the crude product was purified by reversed-phase HPLC (column: X-Select CSH Phenyl-Hexyl 100×30 5u; mobile phase: [H₂O (0.04% HCl)-ACN: THF=1:1]; gradient: 50%-90% B over 14.0 min) to give Compound 954 (101.41 mg, 105.89 μmol, 19.49% yield, 98.22% purity, 0.8 HCl) as a colorless oil.

LCMS [M+1]⁺=911.8

¹H NMR (400 MHz, CHLOROFORM-d) δ=11.93 (br s, 1H), 7.38-7.27 (m, 5H), 4.42 (br s, 1H), 4.25-3.96 (m, 4H), 3.81-3.63 (m, 1H), 3.61-3.29 (m, 6H), 3.28-2.96 (m, 4H), 2.79 (br dd, J=2.0, 6.0 Hz, 1H), 2.42-2.07 (m, 2H), 1.54-1.32 (m, 14H), 1.31-1.09 (m, 56H), 0.89 (t, J=6.8 Hz, 12H).

### Example 7. Lipid Screening by In Vivo Expression Evaluation in Lipid Nanoparticle Formulations

In this example, messenger RNA molecules encoding hEPO proteins were formulated in lipid nanoparticles for delivery in vivo. The lipid nanoparticle formulations comprised of a lipid composition of ionizable lipid:helper lipid:cholesterol:DMG-PEG2k at 50:10:38.5:1.5 mol %. The lipid mixture in ethanol was mixed with hEPO mRNA in RNA acidifying buffer (10 mM citrate, pH 4) at an ionizable-lipid-nitrogen-to-RNA-phosphate ratio (N:P) of 6 using a microfluidic device (Precision NanoSystems, Inc.) at a combined flow rate of 10 mL/min (7.5 mL/min for aqueous buffer, RNA and 2.5 mL/min for ethanol, lipid mix). The resulting particles were neutralized by buffer exchange into Dulbecco's phosphate buffer solution via PD-10 desalting column. The neutralized particles were concentrated using 100 kDa AMICON® Ultra centrifugal filters and sterile filtered using 0.2 um syringe filters. Samples were then characterized and diluted as needed.

LNPs were characterized by dynamic light scattering (DLS) measurement for measuring its hydrodynamic radius and polydispersity index (PDI). Encapsulation efficiency (EE %) and total RNA concentration were quantitated using a fluorescence based Ribogreen assay. The measurements for each of the LNPs are shown in Table 1 below.

TABLE 1
LNP Formulations for in vivo studies

| | | | | | | Zeta | |
|---|---|---|---|---|---|---|---|
| Ionizable | lipid:DSPC:Cholesterol:DMG-PEG2k | | N:P | Size | | potential | Encapsulation |
| Lipid | (mol %) | mRNA | ratio | (nm) | PDI | (mV) | Efficiency % |
| SM-102 | 50:10:38.5:1.5 | hEPO mRNA | 6 | 81.8 | 0.074 | −3.729 | 94.9 |
| Compound 101 | 50:10:38.5:1.5 | hEPO mRNA | 6 | 62.6 | 0.088 | 4.394 | 97.9 |
| Compound 103 | 50:10:38.5:1.5 | hEPO mRNA | 6 | 77.3 | 0.092 | 9.778 | 97.5 |
| Compound 104 | 50:10:38.5:1.5 | hEPO mRNA | 6 | 74.7 | 0.441 | −2.361 | 94.4 |
| Compound 105 | 50:10:38.5:1.5 | hEPO mRNA | 6 | 72.6 | 0.217 | −4.852 | 91.3 |
| Compound 107 | 50:10:38.5:1.5 | hEPO mRNA | 6 | 100.4 | null | 5.484 | 93 |
| Compound 109 | 50:10:38.5:1.5 | hEPO mRNA | 6 | 90.7 | 0.557 | 8.911 | 97.6 |
| Compound 110 | 50:10:38.5:1.5 | hEPO mRNA | 6 | 57.7 | 0.069 | −4.943 | 98 |
| Compound 111 | 50:10:38.5:1.5 | hEPO mRNA | 6 | 93.4 | 0.223 | −3.357 | 95.9 |
| Compound 113 | 50:10:38.5:1.5 | hEPO mRNA | 6 | 79.1 | 0.126 | −2.878 | 95.2 |
| Compound 115 | 50:10:38.5:1.5 | hEPO mRNA | 6 | 70.8 | 0.291 | −2.403 | 89.5 |
| Compound 116 | 50:10:38.5:1.5 | hEPO mRNA | 6 | 79.5 | null | −2.839 | 91.8 |
| Compound 117 | 50:10:38.5:1.5 | hEPO mRNA | 6 | 61.6 | 0.07 | −5.265 | 96.7 |
| Compound 121 | 50:10:38.5:1.5 | hEPO mRNA | 6 | 105.4 | null | 8.346 | 93.1 |
| Compound 122 | 50:10:38.5:1.5 | hEPO mRNA | 6 | 71.8 | 0.259 | 6.541 | 94.2 |
| Compound 123 | 50:10:38.5:1.5 | hEPO mRNA | 6 | 83.3 | 0.338 | −3.45 | 97 |
| Compound 125 | 50:10:38.5:1.5 | hEPO mRNA | 6 | 109.4 | 0.46 | null | 99.4 |
| Compound 127 | 50:10:38.5:1.5 | hEPO mRNA | 6 | 85.9 | 0.387 | 6.011 | 89.5 |
| Compound 128 | 50:10:38.5:1.5 | hEPO mRNA | 6 | 77.8 | 0.058 | 6.43 | 95.2 |
| Compound 129 | 50:10:38.5:1.5 | hEPO mRNA | 6 | 93.8 | null | null | 99.7 |
| Compound 132 | 50:10:38.5:1.5 | hEPO mRNA | 6 | 75.4 | 0.15 | −4.36 | 92.9 |
| Compound 134 | 50:10:38.5:1.5 | hEPO mRNA | 6 | 87.1 | 0.321 | 6.281 | 90.5 |
| Compound 135 | 50:10:38.5:1.5 | hEPO mRNA | 6 | 111.3 | 0.228 | 2.909 | 89.8 |
| Compound 130 | 50:10:38.5:1.5 | hEPO mRNA | 6 | 74.2 | 0.146 | −5.059 | 95.1 |

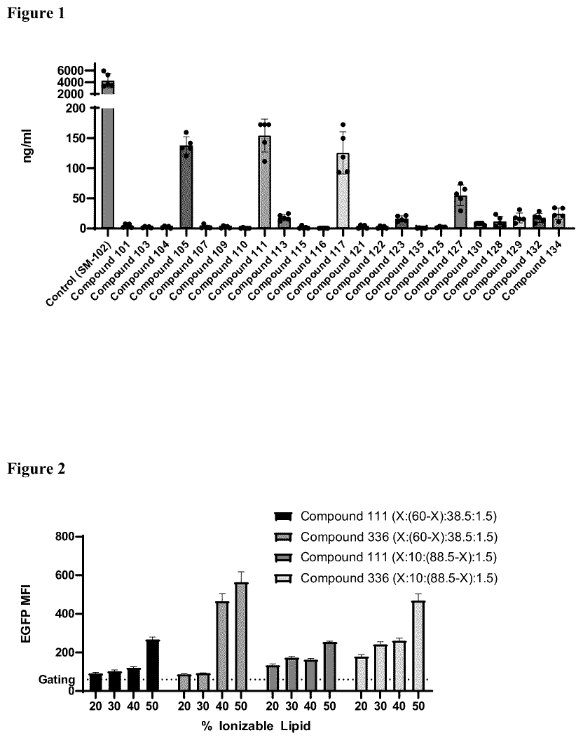

The in vivo studies were performed in C₅₇BL/6 female mice at 6 to 8 weeks weighing in at approximately 20 g. The LNPs formulated with different ionizable lipids at 0.2 mg/kg of hEPO mRNA were administered by tail vein injection and animals were euthanized at 6 h post-administration for blood serum sample collection. The hEPO levels from the samples were analyzed and cross-compared by enzyme-linked immunoassay (ELISA) according to manufacturer's protocol. The hEPO expression levels for each of the LNPs was compared to a control LNP comprising SM102 (DC Chemicals Cat. No. DC52025) (FIG. 1).

### Example 8: In Vitro Evaluation of Lipids with Hydroxyethyl Modifications and Compositional Modifications in LNPs

In this example, messenger RNA molecules encoding EGFP proteins were formulated in lipid nanoparticles for delivery in vitro. The lipid nanoparticle formulations comprised of ionizable lipids at 50, 40, 30, or 20 mol % of which were backfilled with either DSPC or cholesterol while DMG-PEG2000 was fixed at 1.5%. The lipid mixture in ethanol was mixed with EGFP mRNA in RNA acidifying buffer (10 mM citrate, pH 4) at an ionizable-lipid-nitrogen-to-RNA-phosphate ratio (N:P) of 6, 4.8, 3.6, or 2.4 using a microfluidic device (Precision NanoSystems, Inc.) at a combined flow rate of 10 mL/min (7.5 mL/min for aqueous buffer, RNA and 2.5 mL/min for ethanol, lipid mix). The resulting particles were neutralized by buffer exchange into Dulbecco's phosphate buffer solution via PD-10 desalting column. The neutralized particles were concentrated using 100 kDa AMICON® Ultra centrifugal filters and sterile filtered using 0.2 um syringe filters. Resulting LNPs were characterized by dynamic light scattering (DLS) measurement for measuring its hydrodynamic radius and polydispersity index (PDI) and the Zeta potential was measured using a Zetasizer (Malvern). Encapsulation efficiency (EE %) and total RNA concentration were quantitated using a fluorescence based Ribogreen assay. The measurements for each of the LNPs are shown in Table 2 below.

TABLE 2
LNP Formulations for in vitro studies

| | | | | | | Zeta | |
|---|---|---|---|---|---|---|---|
| Ionizable | lipid:DSPC:Cholesterol:DMG-PEG2k | | N:P | Size | | potential | Encapsulation |
| Lipid | (mol %) | mRNA | ratio | (nm) | PDI | (mV) | Efficiency % |
| Compound 111 | 50:10:38.5:1.5 | EGFP | 6 | 106 | 0.253 | −6.678 | 99.8 |
| Compound 111 | 40:20:38.5:1.5 | EGFP | 4.8 | 48.2 | 0.136 | −8.503 | 99.6 |
| Compound 111 | 30:30:38.5:1.5 | EGFP | 3.6 | 57 | 0.117 | −9.169 | 99.2 |
| Compound 111 | 20:40:38.5:1.5 | EGFP | 2.4 | 69.2 | 0.095 | −8.876 | 99.3 |
| Compound 311 | 50:10:38.5:1.5 | EGFP | 6 | 61.7 | 0.094 | −8.861 | 98.6 |
| Compound 311 | 40:20:38.5:1.5 | EGFP | 4.8 | 52.3 | 0.13 | −12.12 | 97.8 |
| Compound 311 | 30:30:38.5:1.5 | EGFP | 3.6 | 76.4 | 0.073 | −10.25 | 97.9 |
| Compound 311 | 20:40:38.5:1.5 | EGFP | 2.4 | 87.9 | 0.067 | −7.495 | 98.2 |
| Compound 111 | 50:10:38.5:1.5 | EGFP | 6 | 110.9 | 0.235 | −2.7 | 99.4 |
| Compound 111 | 40:10:48.5:1.5 | EGFP | 4.8 | 44.1 | 0.038 | −6.9 | 99.7 |
| Compound 111 | 30:10:58.5:1.5 | EGFP | 3.6 | 46.5 | 0.082 | −4.4 | 99.8 |
| Compound 111 | 20:10:68.5:1.5 | EGFP | 2.4 | 57.5 | 0.129 | −6.2 | 99.7 |
| Compound 311 | 50:10:38.5:1.5 | EGFP | 6 | 54.9 | 0.074 | −10.9 | 99.5 |
| Compound 311 | 40:10:48.5:1.5 | EGFP | 4.8 | 49.6 | 0.111 | −6.1 | 99.4 |
| Compound 311 | 30:10:58.5:1.5 | EGFP | 3.6 | 52.2 | 0.098 | −7.1 | 99.2 |
| Compound 311 | 20:10:68.5:1.5 | EGFP | 2.4 | 59.5 | 0.254 | −12.1 | 99 |

Cultured Jurkat cells grown in a 6-well plate were treated with LNPs containing EGFP mRNA at a dosage of 0.5 ug EGFP mRNA per 300,000 cells for 2 hours. After initial treatment, cells were washed and replenished with fresh cell media then further incubated for 22 hours. EGFP expression was detected by flow cytometry and the median fluorescence intensity is reported for comparative analyses (FIG. 2).

### Example 9: In Vivo Expression Evaluation of Chemically Modified Lipids in Lipid Nanoparticle Formulations

In this example, messenger RNA molecules encoding hEPO proteins were formulated in LNPs for delivery in vivo. Isomeric lipids (compound 201 and 202) and hydroxyethyl functionalized lipids (compounds 305, 311, and 317) were directly compared against the precursor lipids (compound 105, 107, 111, 113, and 117) in their LNP forms.

The LNP formulations comprised of a lipid composition of lipid:helper lipid:cholesterol:DMG-PEG2k at 50:10:38.5:1.5 mol %. The lipid mixture in ethanol was mixed with hEPO mRNA in RNA acidifying buffer (10 mM citrate, pH 4) at an ionizable-lipid-nitrogen-to-RNA-phosphate ratio (N:P) of 6 using a microfluidic device (Precision NanoSystems, Inc.) at a combined flow rate of 10 mL/min (7.5 mL/min for aqueous buffer, RNA and 2.5 mL/min for ethanol, lipid mix). The resulting particles were neutralized by buffer exchange into Dulbecco's phosphate buffer solution via PD-10 desalting column. The neutralized particles were concentrated using 100 kDa AMICON® Ultra centrifugal filters and sterile filtered using 0.2 um syringe filters. Resulting LNPs were characterized by dynamic light scattering (DLS) measurement for measuring its hydrodynamic radius and polydispersity index (PDI) and the Zeta potential was measured using a Zetasizer (Malvern). Encapsulation efficiency (EE %) and total RNA concentration were quantitated using a fluorescence based Ribogreen assay. The formulation and measurements for each of the LNPs are shown in Table 3 below.

TABLE 3
LNP Formulations for in vivo studies

| | | | | | | Zeta | |
|---|---|---|---|---|---|---|---|
| Ionizable | lipid:DSPC:Cholesterol:DMG-PEG2k | | N:P | Size | | potential | Encapsulation |
| Lipid | (mol %) | mRNA | ratio | (nm) | PDI | (mV) | Efficiency % |
| Compound 105 | 50:10:38.5:1.5 | hEPO mRNA | 6 | 162.2 | 0.256 | null | 99.9 |
| Compound 305 | 50:10:38.5:1.5 | hEPO mRNA | 6 | 53.3 | 0.051 | null | 99.3 |
| Compound 111 | 50:10:38.5:1.5 | hEPO mRNA | 6 | 100.9 | 0.27 | null | 98.1 |
| Compound 311 | 50:10:38.5:1.5 | hEPO mRNA | 6 | 66 | 0.113 | null | 98.8 |
| Compound 117 | 50:10:38.5:1.5 | hEPO mRNA | 6 | 70.6 | 0.071 | null | 95.1 |
| Compound 311 | 50:10:38.5:1.5 | hEPO mRNA | 6 | 56.3 | 0.052 | null | 95.9 |
| Compound 107 | 50:10:38.5:1.5 | hEPO mRNA | 6 | 60.6 | 0.016 | null | 99.9 |
| Compound 201 | 50:10:38.5:1.5 | hEPO mRNA | 6 | 63.1 | 0.032 | null | 99.9 |
| Compound 113 | 50:10:38.5:1.5 | hEPO mRNA | 6 | 61.4 | 0.068 | null | 99.7 |
| Compound 202 | 50:10:38.5:1.5 | hEPO mRNA | 6 | 60.7 | 0.033 | null | 99.7 |

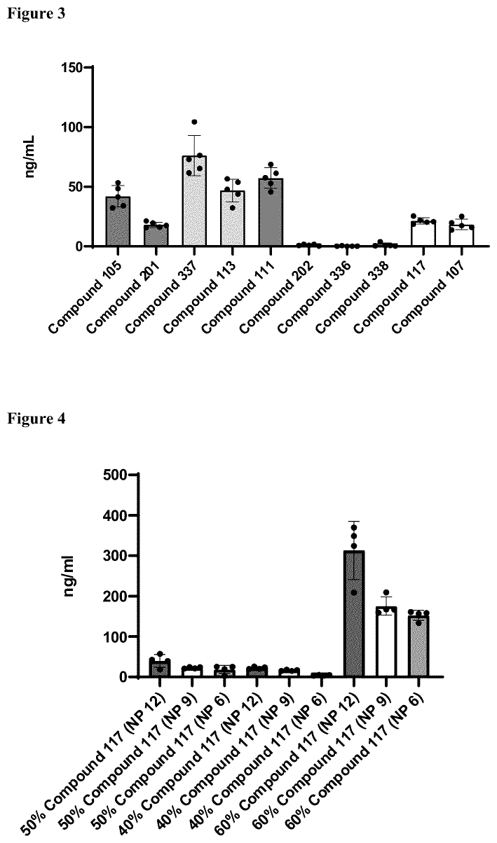

The in vivo studies were performed in C₅₇BL/6 female mice at 6 to 8 weeks weighing in at approximately 20 g. The LNPs formulated with different ionizable lipids at 0.2 mg/kg of hEPO mRNA were administered by tail vein injection and animals were euthanized at 6 h post-administration for blood serum sample collection. The hEPO levels from the samples were analyzed and cross-compared by enzyme-linked immunoassay (ELISA) according to manufacturer's protocol (FIG. 3).

### Example 10: In Vivo Expression of Phenylalanine Lipids in Different Lipid Nanoparticle Compositions

In this example, messenger RNA molecules encoding hEPO proteins were formulated in LNPs for delivery in vivo. Phenylalanine lipids were formulated into three different formulation strategies of which each were prepared at N:P ratios of 6, 9, and 12. Standard lipid composition of ionizable lipid:helper lipid:cholesterol:DMG-PEG2k at 50:10:38.5:1.5 mol % was used along with a ratio of 40:10:48.5:1.5 and 60:0:38.5:1.5. The nine different lipid mixtures in ethanol were mixed with hEPO mRNA in RNA acidifying buffer (10 mM citrate, pH 4) using a microfluidic device (Precision NanoSystems, Inc.) at a combined flow rate of 10 mL/min (7.5 mL/min for aqueous buffer, RNA and 2.5 mL/min for ethanol, lipid mix). The resulting particles were neutralized by buffer exchange into Dulbecco's phosphate buffer solution via PD-10 desalting column. The neutralized particles were concentrated using 100 kDa AMICON® Ultra centrifugal filters and sterile filtered using 0.2 um syringe filters. Resulting LNPs were characterized by dynamic light scattering (DLS) measurement for measuring its hydrodynamic radius and polydispersity index (PDI) and the Zeta potential was measured using a Zetasizer (Malvern). Encapsulation efficiency (EE %) and total RNA concentration were quantitated using a fluorescence based Ribogreen assay, and the apparent pKa of the LNPs was measured by a fluorescence based TNS assay. The formulation and measurements for each of the LNPs are shown in Table 4 below.

TABLE 4
LNP formulations for in vivo studies

| | | | | | | Zeta | | |
|---|---|---|---|---|---|---|---|---|
| Ionizable | Lipid:DSPC:Cholesterol:DMG-PEG2k | | N:P | Size | | potential | Encapsulation | |
| Lipid | (mol %) | mRNA | ratio | (nm) | PDI | (mV) | Efficiency % | pKa |
| Compound 117 | 50:10:38.5:1.5 | hEPO | 6 | 62.2 | 0.134 | −4.369 | 96.3 | 4.005 |
| Compound 117 | 50:10:38.5:1.5 | hEPO | 9 | 65.3 | 0.365 | −2.051 | 98.6 | 4.019 |
| Compound 117 | 50:10:38.5:1.5 | hEPO | 12 | 67.8 | 0.515 | −4.408 | 98.2 | 3.227 |
| Compound 117 | 40:10:48.5:1.5 | hEPO | 6 | 56.4 | 0.204 | −0.6716 | 87.7 | 3.568 |
| Compound 117 | 40:10:48.5:1.5 | hEPO | 9 | 67.8 | 0.354 | −1.95 | 98 | 3.999 |
| Compound 117 | 40:10:48.5:1.5 | hEPO | 12 | 75.7 | 0.119 | −3.302 | 98.3 | 4.234 |
| Compound 117 | 60:0:38.5:1.5 | hEPO | 6 | 125 | 0.089 | −5.393 | 90.2 | null |
| Compound 117 | 60:0:38.5:1.5 | hEPO | 9 | 123 | 0.018 | −5.043 | 92.3 | null |
| Compound 117 | 60:0:38.5:1.5 | hEPO | 12 | 55 | 0.263 | −5.125 | 94.8 | null |

The in vivo studies were performed in C₅₇BL/6 female mice at 6 to 8 weeks weighing in at approximately 20 g. The LNPs formulated with different ionizable lipids at 0.2 mg/kg of hEPO mRNA were administered by tail vein injection and animals were euthanized at 6 h post-administration for blood serum sample collection. The hEPO levels from the samples were analyzed and cross-compared by enzyme-linked immunoassay (ELISA) according to manufacturer's protocol (FIG. 4).

### Example 11: In Vivo Expression Evaluation of Glycine, Alanine, and Phenylalanine Lipids with Different Lipid Tail Structures in Lipid Nanoparticle Formulations

In this example, messenger RNA molecules encoding hEPO proteins were formulated in LNPs for delivery in vivo. The lipid nanoparticle formulations comprised of a lipid composition of lipid:helper lipid:cholesterol:DMG-PEG2k at 50:10:38.5:1.5 mol % or 60:0:38.5:1.5 mol %. The lipid mixture in ethanol was mixed with hEPO mRNA in RNA acidifying buffer (10 mM citrate, pH 4) at a N-to-P ratio of 6 using a microfluidic device (Precision NanoSystems, Inc.) at a combined flow rate of 10 mL/min (7.5 mL/min for aqueous buffer, RNA and 2.5 mL/min for ethanol, lipid mix). The resulting particles were neutralized by buffer exchange into Dulbecco's phosphate buffer solution via PD-10 desalting column. The neutralized particles were concentrated using 100 kDa AMICON® Ultra centrifugal filters and sterile filtered using 0.2 um syringe filters. Resulting LNPs were characterized by dynamic light scattering (DLS) measurement for measuring its hydrodynamic radius and polydispersity index (PDI) and the Zeta potential was measured using a Zetasizer (Malvern). Encapsulation efficiency (EE %) and total RNA concentration were quantitated using a fluorescence based Ribogreen assay, and the apparent pKa of the LNPs was measured by a fluorescence based TNS assay. The formulation and measurements for each of the LNPs are shown in Table 5 below.

TABLE 5
LNP formulations for in vivo studies

| | | | | | | Zeta | | |
|---|---|---|---|---|---|---|---|---|
| Ionizable | Lipid:DSPC:Cholesterol:DMG-PEG2k | | N:P | Size | | potential | Encapsulation | |
| Lipid | (mol %) | mRNA | ratio | (nm) | PDI | (mV) | Efficiency % | pKa |
| Compound 601 | 50:10:38.5:1.5 | hEPO | 6 | 83.8 | 0.185 | −4.878 | 99.8 | 4.48 |
| Compound 602 | 50:10:38.5:1.5 | hEPO | 6 | 84.8 | 0.337 | −5.94 | 99.1 | 3.73 |
| Compound 603 | 50:10:38.5:1.5 | hEPO | 6 | 71.7 | 0.213 | −3.116 | 98.4 | 5.15 |
| Compound 604 | 50:10:38.5:1.5 | hEPO | 6 | 208.5 | 0.102 | −7.347 | 99.3 | 3.36 |
| Compound 605 | 50:10:38.5:1.5 | hEPO | 6 | 68.4 | 0.399 | −6.585 | 99.8 | 3.34 |
| Compound 606 | 50:10:38.5:1.5 | hEPO | 6 | 57.2 | 0.457 | −2.662 | 99.9 | 4.28 |
| Compound 607 | 50:10:38.5:1.5 | hEPO | 6 | 68.9 | null | −12.44 | 99.7 | null |
| Compound 608 | 50:10:38.5:1.5 | hEPO | 6 | 77.1 | 0.13 | −4.462 | 97.7 | 3.79 |
| Compound 609 | 50:10:38.5:1.5 | hEPO | 6 | 98 | null | −4.452 | 99.8 | 5.56 |
| Compound 610 | 50:10:38.5:1.5 | hEPO | 6 | 77.3 | 0.202 | −1.535 | 99.7 | 7.28 |
| Compound 611 | 50:10:38.5:1.5 | hEPO | 6 | 155.4 | 0.24 | −5.841 | 99.3 | 5.25 |
| Compound 612 | 50:10:38.5:1.5 | hEPO | 6 | 69.9 | 0.403 | −3.371 | 99.8 | 5.79 |
| Compound 613 | 50:10:38.5:1.5 | hEPO | 6 | 53.7 | 0.236 | −6.511 | 99.8 | 7.03 |
| Compound 614 | 50:10:38.5:1.5 | hEPO | 6 | 79.1 | 0.443 | −8.485 | 99.7 | null |
| Compound 615 | 50:10:38.5:1.5 | hEPO | 6 | 104.2 | 0.408 | −11.83 | 98.7 | 5.94 |
| Compound 616 | 50:10:38.5:1.5 | hEPO | 6 | 67.5 | 0.435 | −13.88 | 98.6 | 5.69 |
| Compound 617 | 50:10:38.5:1.5 | hEPO | 6 | 63.7 | 0.32 | −5.824 | 99.1 | 6 |
| Compound 618 | 50:10:38.5:1.5 | hEPO | 6 | 53.9 | 0.304 | −13.69 | 97.9 | 3.68 |
| Compound 619 | 50:10:38.5:1.5 | hEPO | 6 | 60.7 | 0.183 | −11.63 | 98.1 | 4.26 |
| Compound 620 | 50:10:38.5:1.5 | hEPO | 6 | 56.7 | 0.23 | −5.044 | 99 | 5.19 |
| Compound 621 | 50:10:38.5:1.5 | hEPO | 6 | 49.4 | 0.332 | −10.9 | 98.9 | 5.82 |
| Compound 105 | 50:10:38.5:1.5 | hEPO | 6 | 166.9 | 0.104 | −5.091 | 99.6 | 4.58 |
| Compound 305 | 50:10:38.5:1.5 | hEPO | 6 | 59.3 | 0.05 | −0.5189 | 99.5 | 6.66 |
| Compound 105 | 60:0:38.5:1.5 | hEPO | 6 | 146.3 | 0.198 | −8.315 | 99.3 | null |
| Compound 305 | 60:0:38.5:1.5 | hEPO | 6 | 52.7 | 0.098 | −2.027 | 99.5 | 6.75 |
| Compound 604 | 60:0:38.5:1.5 | hEPO | 6 | 236.6 | 0.15 | −3.753 | 98 | null |
| Compound 611 | 60:0:38.5:1.5 | hEPO | 6 | 55.6 | 0.244 | −5.555 | 98.4 | null |
| Compound 618 | 60:0:38.5:1.5 | hEPO | 6 | 211.9 | 0.116 | −17.91 | 92.8 | 4.15 |

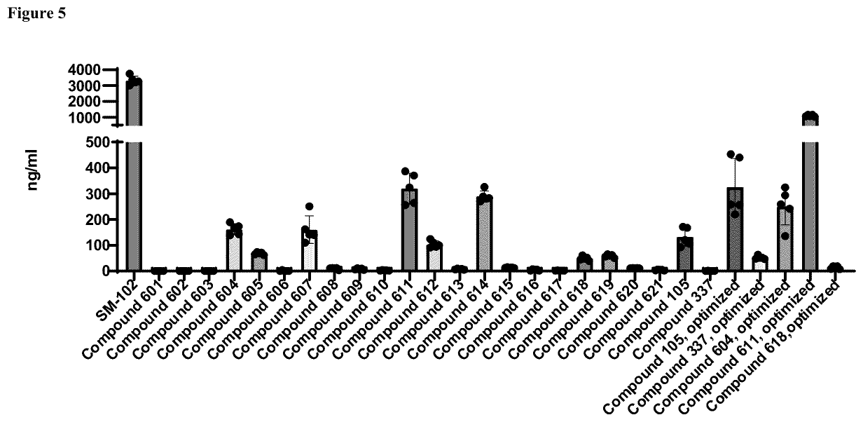

The in vivo studies were performed in C57BL/6 female mice at 6 to 8 weeks weighing in at approximately 20 g. The LNPs formulated with different ionizable lipids at 0.2 mg/kg of hEPO mRNA were administered by tail vein injection and animals were euthanized at 6 h post-administration for blood serum sample collection. The hEPO levels from the samples were analyzed and cross-compared by enzyme-linked immunoassay (ELISA) according to manufacturer's protocol. The hEPO expression levels for each of the LNPs was compared to a control LNP comprising SM102 (DC Chemicals Cat. No. DC52025) (FIG. 5).

### Example 12: In Vivo Expression Evaluation of Beta- and Gamma-Amino Acid Lipids in Lipid Nanoparticle Formulations

In this example, messenger RNA molecules encoding hEPO proteins were formulated in LNPs for delivery in vivo. The lipid nanoparticle formulations comprised of a lipid composition of lipid:helper lipid:cholesterolDMG-PEG2k at 50:10:38.5:1.5 mol % or 60:0:38.5:1.5 mol %. The lipid mixture in ethanol was mixed with hEPO mRNA in RNA acidifying buffer (10 mM citrate, pH 4) at a N-to-P ratio of 6 using a microfluidic device (Precision NanoSystems, Inc.) at a combined flow rate of 10 mL/min (7.5 mL/min for aqueous buffer, RNA and 2.5 mL/min for ethanol, lipid mix). The resulting particles were neutralized by buffer exchange into Dulbecco's phosphate buffer solution via PD-10 desalting column. The neutralized particles were concentrated using 100 kDa AMICON® Ultra centrifugal filters and sterile filtered using 0.2 um syringe filters. Resulting LNPs were characterized by dynamic light scattering (DLS) measurement for measuring its hydrodynamic radius and polydispersity index (PDI) and the Zeta potential was measured using a Zetasizer (Malvern). Encapsulation efficiency (EE %) and total RNA concentration were quantitated using a fluorescence based Ribogreen assay, and the apparent pKa of the LNPs was measured by a fluorescence based TNS assay. The formulation and measurements for each of the LNPs are shown in Table 6 below.

TABLE 6
LNP formulations for in vivo studies

| | | | | | | Zeta | | |
|---|---|---|---|---|---|---|---|---|
| Ionizable | lipid:DSPC:Cholesterol:DMG-PEG2k | | N:P | Size | | potential | Encapsulation | |
| Lipid | (mol %) | mRNA | ratio | (nm) | PDI | (mV) | Efficiency % | pKa |
| Dlin-MC3-DMA | 50:10:38.5:1.5 | hEPO | 6 | 79.2 | 0.051 | −1.608 | 90 | 5.21 |
| Compound 801 | 50:10:38.5:1.5 | hEPO | 6 | 105.8 | 0.436 | −1.5 | 98.5 | 5.21 |
| Compound 802 | 50:10:38.5:1.5 | hEPO | 6 | 104.5 | 0.224 | −1.8 | 98.4 | 3.97 |
| Compound 804 | 50:10:38.5:1.5 | hEPO | 6 | 96 | 0.281 | −1.7 | 98.3 | 4.8 |
| Compound 805 | 50:10:38.5:1.5 | hEPO | 6 | 110.2 | 0.214 | −0.2 | 98.3 | 3.86 |
| Compound 807 | 50:10:38.5:1.5 | hEPO | 6 | 77.7 | 0.126 | −1.9 | 98.3 | 5.26 |
| Compound 808 | 50:10:38.5:1.5 | hEPO | 6 | 96.3 | 0.114 | −0.3 | 97 | 5.58 |
| Compound 801 | 60:0:38.5:1.5 | hEPO | 6 | 93.6 | 0.169 | 1.6 | 98.4 | null |
| Compound 802 | 60:0:38.5:1.5 | hEPO | 6 | 87.6 | 0.195 | 0.1 | 98.4 | null |
| Compound 804 | 60:0:38.5:1.5 | hEPO | 6 | 81.1 | 0.147 | 2.8 | 99.9 | 9.06 |
| Compound 805 | 60:0:38.5:1.5 | hEPO | 6 | 84.7 | 0.137 | 3 | 99.9 | 10.4 |
| Compound 807 | 60:0:38.5:1.5 | hEPO | 6 | 114.7 | 0.079 | −0.6 | 99.7 | 7.57 |
| Compound 808 | 60:0:38.5:1.5 | hEPO | 6 | 131.4 | 0.012 | −0.4 | 99.2 | 6.71 |

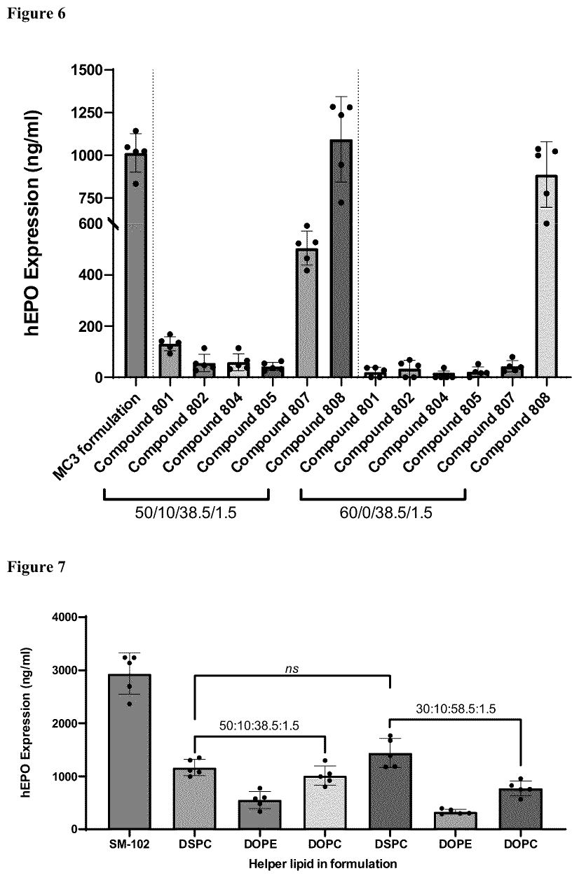

The in vivo studies were performed in C57BL/6 female mice at 6 to 8 weeks weighing in at approximately 20 g. The LNPs formulated with different ionizable lipids at 0.2 mg/kg of hEPO mRNA were administered by tail vein injection and animals were euthanized at 6 h post-administration for blood serum sample collection. The hEPO levels from the samples were analyzed and cross-compared by enzyme-linked immunoassay (ELISA) according to manufacturer's protocol. The hEPO expression levels for each of the LNPs was compared to a control LNP comprising MC3 (BroadPharm Cat. No. BP-25497) (FIG. 6).

### Example 13: In Vivo Expression Evaluation of Beta Dimethyl Glycine Lipids in Lipid Nanoparticle Formulations with Varying Helper Lipids

In this example, messenger RNA molecules encoding hEPO proteins were formulated in LNPs with varying helper lipids for delivery in vivo. The lipid nanoparticle formulations comprised of a lipid composition of lipid:helper lipid:cholesterol:DMG-PEG2k at 50:10:38.5:1.5 mol % or 30:10:38.5:1.5 mol % and the additional helper lipids included in this study were 1,2-dioleoyl-sn-glycero-3-phosphatidylethanolamine (DOPE) and 1,2-dioleoyl-sn-glycero-3-phosphatidylcholine (DOPC). The lipid mixture in ethanol was mixed with hEPO mRNA in RNA acidifying buffer (10 mM citrate, pH 4) at a N-to-P ratio of 6 using a microfluidic device (Precision NanoSystems, Inc.) at a combined flow rate of 10 mL/min (7.5 mL/min for aqueous buffer, RNA and 2.5 mL/min for ethanol, lipid mix). The resulting particles were neutralized by buffer exchange into Dulbecco's phosphate buffer solution via PD-10 desalting column. The neutralized particles were concentrated using 100 kDa AMICON® Ultra centrifugal filters and sterile filtered using 0.2 um syringe filters. Resulting LNPs were characterized by dynamic light scattering (DLS) measurement for measuring its hydrodynamic radius and polydispersity index (PDI) and the Zeta potential was measured using a Zetasizer (Malvern). Encapsulation efficiency (EE %) and total RNA concentration were quantitated using a fluorescence based Ribogreen assay, and the apparent pKa of the LNPs was measured by a fluorescence based TNS assay. The formulation and measurements for each of the LNPs are shown in Table 7 below.

TABLE 7
LNP formulations for in vivo studies

| | Compound 808:Helper | | | | | Zeta | | |
|---|---|---|---|---|---|---|---|---|
| | Lipid:Cholesterol:DMG- | | N:P | Size | | potential | Encapsulation | |
| Helper Lipid | PEG2k (mol %) | mRNA | ratio | (nm) | PDI | (mV) | Efficiency % | pKa |
| SM-102/DSPC | 50:10:38.5:1.5 | hEPO | 6 | 75.3 | 0.041 | −17.15 | 98.7 | 6.1 |
| DSPC | 50:10:38.5:1.5 | hEPO | 6 | 114.4 | 0.13 | −7.7 | 98.8 | 6.1 |
| DOPE | 50:10:38.5:1.5 | hEPO | 6 | 134.5 | 0.214 | −7.52 | 99.5 | 6.6 |
| DOPC | 50:10:38.5:1.5 | hEPO | 6 | 99.1 | 0.131 | −8.233 | 98.8 | 6.7 |
| DSPC | 30:10:58.5:1.5 | hEPO | 6 | 93.8 | 0.033 | −7.184 | 99.6 | 5.3 |
| DOPE | 30:10:58.5:1.5 | hEPO | 6 | 79.1 | 0.105 | −10.16 | 99.7 | 4.8 |
| DOPC | 30:10:58.5:1.5 | hEPO | 6 | 120.1 | 0.113 | −9.115 | 99.5 | 6.4 |

The in vivo studies were performed in C₅₇BL/6 female mice at 6 to 8 weeks weighing in at approximately 20 g. The LNPs formulated with different ionizable lipids at 0.2 mg/kg of hEPO mRNA were administered by tail vein injection and animals were euthanized at 6 h post-administration for blood serum sample collection. The hEPO levels from the samples were analyzed and cross-compared by enzyme-linked immunoassay (ELISA) according to manufacturer's protocol. The hEPO expression levels for each of the LNPs was compared to a control LNP comprising SM102 (DC Chemicals Cat. No. DC52025) (FIG. 7).

### Example 14: In Vivo Expression Evaluation of Headgroup Modification on Lipids in Lipid Nanoparticle Formulations

In this example, messenger RNA molecules encoding hEPO proteins were formulated in LNPs for delivery in vivo. The lipid nanoparticle formulations comprised of a lipid composition of a lipid:helper lipid:cholesterol:DMG-PEG2k at 50:10:38.5:1.5 mol %. The lipid mixture in ethanol was mixed with hEPO mRNA in RNA acidifying buffer (10 mM citrate, pH 4) at a N-to-P ratio of 6 using a microfluidic device (Precision NanoSystems, Inc.) at a combined flow rate of 10 mL/min (7.5 mL/min for aqueous buffer, RNA and 2.5 mL/min for ethanol, lipid mix). The resulting particles were neutralized by buffer exchange into Dulbecco's phosphate buffer solution via PD-10 desalting column. The neutralized particles were concentrated using 100 kDa AMICON® Ultra centrifugal filters and sterile filtered using 0.2 um syringe filters. Resulting LNPs were characterized by dynamic light scattering (DLS) measurement for measuring its hydrodynamic radius and polydispersity index (PDI) and the Zeta potential was measured using a Zetasizer (Malvern). Encapsulation efficiency (EE %) and total RNA concentration were quantitated using a fluorescence based Ribogreen assay, and the apparent pKa of the LNPs was measured by a fluorescence based TNS assay. The formulation and measurements for each of the LNPs are shown in Table 8, Table 9, and Table 10 below.

TABLE 8
LNP formulations for in vivo studies

| | | | | | | Zeta | | |
|---|---|---|---|---|---|---|---|---|
| Ionizable | Lipid:DSPC:Cholesterol:DMG-PEG2k | | N:P | Size | | potential | Encapsulation | |
| Lipid | (mol %) | mRNA | ratio | (nm) | PDI | (mV) | Efficiency % | pKa |
| SM-102 | 50:10:38.5:1.5 | hEPO | 6 | 80.3 | 0.042 | −9.7 | 99 | 5.973 |
| Dlin-MC3-DMA | 50:10:38.5:1.5 | hEPO | 6 | 69.3 | 0.028 | −9.2 | 100 | 5.818 |
| Compound 901 | 50:10:38.5:1.5 | hEPO | 6 | 91.4 | 0.076 | −9.9 | 98 | 5.968 |
| Compound 902 | 50:10:38.5:1.5 | hEPO | 6 | 83 | 129 | −101 | 98 | 598 |
| Compound 903 | 50:10:38.5:1.5 | hEPO | 6 | 73.7 | 0.02 | −10.9 | 98 | 5.71 |
| Compound 904 | 50:10:38.5:1.5 | hEPO | 6 | 86.7 | 0.189 | −1.6 | 100 | 6.294 |
| Compound 905 | 50:10:38.5:1.5 | hEPO | 6 | 70.4 | 0.204 | −2.9 | 100 | 6.258 |
| Compound 906 | 50:10:38.5:1.5 | hEPO | 6 | 81.9 | 0.127 | −9.9 | 97 | 5.963 |
| Compound 907 | 50:10:38.5:1.5 | hEPO | 6 | 80.3 | 0.037 | −10.4 | 98 | 5.608 |
| Compound 908 | 50:10:38.5:1.5 | hEPO | 6 | 69.2 | 0.215 | −8 | 100 | 6.252 |
| Compound 909 | 50:10:38.5:1.5 | hEPO | 6 | 72.7 | 0.088 | −11.3 | 95 | 5.689 |
| Compound 910 | 50:10:38.5:1.5 | hEPO | 6 | 98 | 0.116 | −11.1 | 92 | 5.628 |
| Compound 911 | 50:10:38.5:1.5 | hEPO | 6 | 88.1 | 0.073 | −10.5 | 79 | 5.371 |
| Compound 912 | 50:10:38.5:1.5 | hEPO | 6 | 101.9 | 0.047 | −9 | 96 | 5.935 |
| Compound 913 | 50:10:38.5:1.5 | hEPO | 6 | 92 | 0.097 | −11.3 | 94 | 5.841 |
| Compound 914 | 50:10:38.5:1.5 | hEPO | 6 | 73.8 | 0.217 | −12 | 84 | 4.848 |
| Compound 915 | 50:10:38.5:1.5 | hEPO | 6 | 74.3 | 0.167 | −13.4 | 85 | 5.05 |
| Compound 916 | 50:10:38.5:1.5 | hEPO | 6 | 85.5 | 0.118 | −7.2 | 99 | 6.004 |
| Compound 921 | 50:10:38.5:1.5 | hEPO | 6 | 84.8 | 0.122 | −14.7 | 93.6 | 4.798 |
| Compound 922 | 50:10:38.5:1.5 | hEPO | 6 | 74.9 | 0.095 | −13.7 | 93.4 | 4.73 |
| Compound 923 | 50:10:38.5:1.5 | hEPO | 6 | 64.1 | 0.109 | −15.9 | 84.8 | 4.552 |
| Compound 924 | 50:10:38.5:1.5 | hEPO | 6 | 87.2 | 0.142 | −8.7 | 99.9 | N/A |
| Compound 925 | 50:10:38.5:1.5 | hEPO | 6 | 79.6 | 0.146 | −6.1 | 99.9 | N/A |
| Compound 926 | 50:10:38.5:1.5 | hEPO | 6 | 88.5 | 0.102 | −13.1 | 93.4 | 4.786 |
| Compound 927 | 50:10:38.5:1.5 | hEPO | 6 | 87.8 | 0.09 | −12.6 | 87.9 | 4.443 |
| Compound 928 | 50:10:38.5:1.5 | hEPO | 6 | 75.5 | 0.177 | −11.9 | 91.8 | N/A |
| Compound 929 | 50:10:38.5:1.5 | hEPO | 6 | 72.9 | 0.223 | −14.2 | 89.1 | 4.237 |
| Compound 970 | 50:10:38.5:1.5 | hEPO | 6 | 89 | 0.133 | −16.3 | 82.9 | 4.304 |
| Compound 971 | 50:10:38.5:1.5 | hEPO | 6 | 138 | 0.267 | −18.1 | 72.2 | 3.958 |
| Compound 972 | 50:10:38.5:1.5 | hEPO | 6 | 83.6 | 0.095 | −15.5 | 94.6 | 4.935 |
| Compound 973 | 50:10:38.5:1.5 | hEPO | 6 | 94.8 | 0.17 | −13.4 | 86.5 | 4.374 |
| Compound 974 | 50:10:38.5:1.5 | hEPO | 6 | 67.3 | 0.171 | −14.5 | 65.2 | 3.073 |
| Compound 975 | 50:10:38.5:1.5 | hEPO | 6 | 77.9 | 0.135 | −14.4 | 63.7 | 3.449 |
| Compound 976 | 50:10:38.5:1.5 | hEPO | 6 | 62.4 | 0.11 | −12.2 | 93.5 | 4.795 |
| Compound 931 | 50:10:38.5:1.5 | hEPO | 6 | 125.8 | 0.117 | −8.457 | 99.9 | 6.869 |
| Compound 932 | 50:10:38.5:1.5 | hEPO | 6 | 123.7 | 0.155 | −9.4 | 99.9 | 6.92 |
| Compound 933 | 50:10:38.5:1.5 | hEPO | 6 | 110.9 | 0.301 | −12.2 | 99.2 | |
| Compound 935 | 50:10:38.5:1.5 | hEPO | 6 | 111.9 | 0.167 | −9.5 | 99.9 | |
| Compound 936 | 50:10:38.5:1.5 | hEPO | 6 | 110.1 | 0.175 | −10.7 | 99.9 | 6.463 |
| Compound 937 | 50:10:38.5:1.5 | hEPO | 6 | 144 | 0.136 | −9.2 | 99.9 | 6.806 |
| Compound 938 | 50:10:38.5:1.5 | hEPO | 6 | 103.7 | 0.248 | −18.3 | 99.9 | |
| Compound 939 | 50:10:38.5:1.5 | hEPO | 6 | 103.5 | 0.172 | −14.9 | 98.5 | 6.293 |
| Compound 980 | 50:10:38.5:1.5 | hEPO | 6 | 90.1 | 0.159 | −18.1 | 94.9 | 6.314 |
| Compound 982 | 50:10:38.5:1.5 | hEPO | 6 | 120.5 | 0.081 | −14.2 | 99.9 | 7.083 |
| Compound 983 | 50:10:38.5:1.5 | hEPO | 6 | 90.4 | 0.114 | −15.9 | 97.1 | 6.551 |
| Compound 986 | 50:10:38.5:1.5 | hEPO | 6 | 112.9 | 0.088 | −8.2 | 99.9 | 6.956 |
| Compound 942 | 50:10:38.5:1.5 | hEPO | 6 | 96.8 | 0.081 | | 98 | |
| Compound 943 | 50:10:38.5:1.5 | hEPO | 6 | 74.6 | 0.038 | | 95.1 | |
| Compound 946 | 50:10:38.5:1.5 | hEPO | 6 | 84.4 | 0.047 | | 97.6 | |
| Compound 947 | 50:10:38.5:1.5 | hEPO | 6 | 82.7 | 0.05 | | 97.7 | |
| Compound 948 | 50:10:38.5:1.5 | hEPO | 6 | 74.1 | 0.188 | | 99.7 | |
| Compound 949 | 50:10:38.5:1.5 | hEPO | 6 | 79.8 | 0.1 | | 95.1 | |
| Compound 990 | 50:10:38.5:1.5 | hEPO | 6 | 92.6 | 0.074 | | 93 | |
| Compound 992 | 50:10:38.5:1.5 | hEPO | 6 | 99.7 | 0.076 | | 95.7 | |
| Compound 993 | 50:10:38.5:1.5 | hEPO | 6 | 89 | 0.061 | | 94 | |

TABLE 9
Additional LNP formulations for in vivo studies

| | | | | | | Zeta | | |
|---|---|---|---|---|---|---|---|---|
| Ionizable | Lipid:DSPC:Cholesterol:DMG-PEG2k | | N:P | Size | | potential | Encapsulation | |
| Lipid | (mol %) | mRNA | ratio | (nm) | PDI | (mV) | Efficiency % | pKa |
| SM-102 | 50:10:38.5:1.5 | hEPO | 6 | 80.7 | 0.098 | — | 99.8 | 6.361 |
| Compound 961 | 50:10:38.5:1.5 | hEPO | 6 | — | — | — | 99.8 | — |
| Compound 962 | 50:10:38.5:1.5 | hEPO | 6 | 74.9 | 0.132 | −30.33 | 99.6 | 5.17 |
| Compound 963 | 50:10:38.5:1.5 | hEPO | 6 | 76.1 | 0.447 | −41.21 | 99.2 | 5.151 |
| Compound 964 | 50:10:38.5:1.5 | hEPO | 6 | 73.9 | 0.317 | −26.13 | 99.3 | 5.49 |
| Compound 965 | 50:10:38.5:1.5 | hEPO | 6 | 76.3 | 0.216 | −14.27 | 98.6 | 6.251 |
| Compound 966 | 50:10:38.5:1.5 | hEPO | 6 | 89.5 | 0.076 | −36.65 | 97.8 | 4.965 |
| Compound 967 | 50:10:38.5:1.5 | hEPO | 6 | 82.1 | 0.101 | −21.99 | 97.9 | 4.894 |
| Compound 968 | 50:10:38.5:1.5 | hEPO | 6 | 76.7 | 0.368 | −13.15 | 98.2 | 5.838 |
| Compound 969 | 50:10:38.5:1.5 | hEPO | 6 | 70 | 0.15 | −28.83 | 99.4 | 4.556 |
| Compound 960a | 50:10:38.5:1.5 | hEPO | 6 | 77.4 | 0.125 | −28.49 | 99.7 | 4.335 |
| Compound 960b | 50:10:38.5:1.5 | hEPO | 6 | 61.4 | 0.099 | −23.35 | 99.8 | 3.767 |
| Compound 960c | 50:10:38.5:1.5 | hEPO | 6 | 88 | 0.079 | −20.89 | 99.7 | 5.495 |
| Compound 960d | 50:10:38.5:1.5 | hEPO | 6 | 74.5 | 0.133 | −32.3 | 99.5 | 4.517 |
| Compound 960e | 50:10:38.5:1.5 | hEPO | 6 | 73.3 | 0.384 | −45.58 | 99.4 | 3.398 |
| Compound 960f | 50:10:38.5:1.5 | hEPO | 6 | 111.4 | 0.186 | −26.56 | 99.2 | 3.756 |
| Compound 960g | 50:10:38.5:1.5 | hEPO | 6 | 72.6 | 0.254 | −12.1 | 99 | 5.437 |

TABLE 10
Additional LNP formulations for in vivo studies

| | | | | | | Zeta | | |
|---|---|---|---|---|---|---|---|---|
| Ionizable | Lipid:DSPC:Cholesterol:DMG-PEG2k | | N:P | Size | | potential | Encapsulation | |
| Lipid | (mol %) | mRNA | ratio | (nm) | PDI | (mV) | Efficiency % | pKa |
| SM-102 | 50:10:38.5:1.5 | hEPO | 6 | 56.5 | 0.272 | −5.9 | 92.3 | 7.979 |
| ALC-0315 | 50:10:38.5:1.5 | hEPO | 6 | 61.4 | 0.295 | −10.3 | 83.8 | 6.817 |
| Compound 917a | 50:10:38.5:1.5 | hEPO | 6 | 82.8 | 0.124 | −5.5 | 43.4 | 5.974 |
| Compound 917b | 50:10:38.5:1.5 | hEPO | 6 | 163.6 | 0.282 | −1.3 | 98 | 6.325 |
| Compound 917c | 50:10:38.5:1.5 | hEPO | 6 | 106.9 | 0.122 | −4.5 | 84 | 6.13 |
| Compound 917d | 50:10:38.5:1.5 | hEPO | 6 | 122 | 0.096 | −3.3 | 60.3 | 6.192 |
| Compound 917e | 50:10:38.5:1.5 | hEPO | 6 | 52.5 | 0.222 | −4.2 | 95.4 | 7.052 |
| Compound 917f | 50:10:38.5:1.5 | hEPO | 6 | 51.5 | 0.314 | −11.1 | 91.9 | 7.342 |
| Compound 917g | 50:10:38.5:1.5 | hEPO | 6 | 49.2 | 0.281 | −10.2 | 93.5 | 6.369 |
| Compound 917h | 50:10:38.5:1.5 | hEPO | 6 | 89.6 | 0.137 | −8.2 | 91.4 | 6.256 |
| Compound 917i | 50:10:38.5:1.5 | hEPO | 6 | 84.6 | 0.059 | −6.6 | 87 | 5.788 |

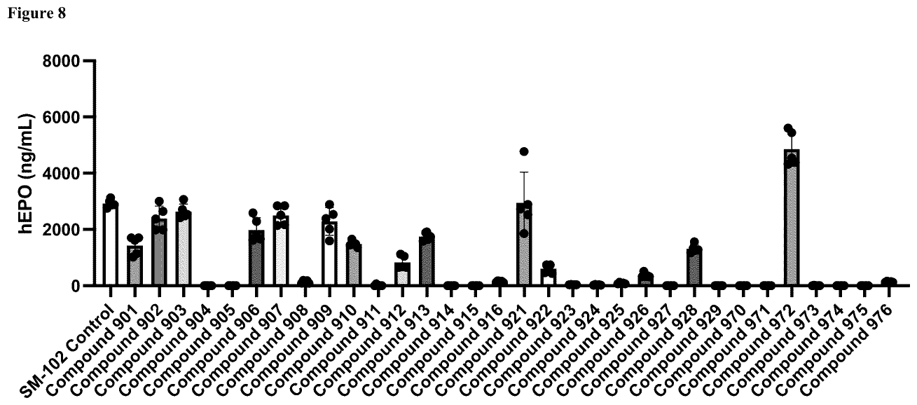

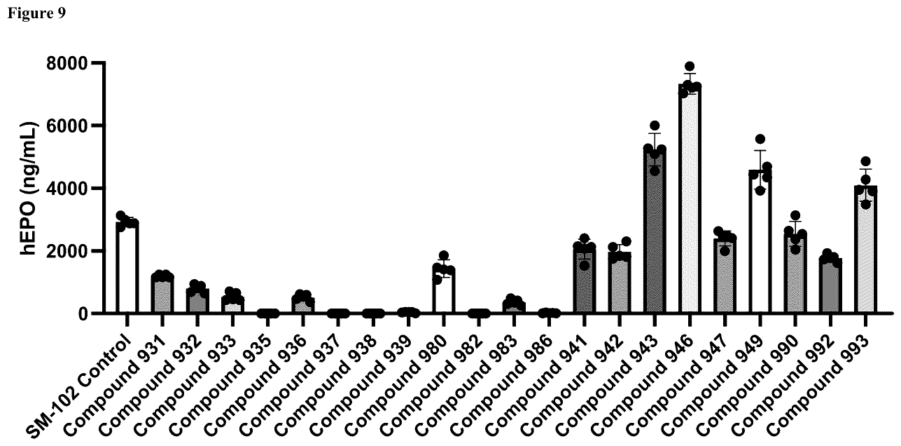

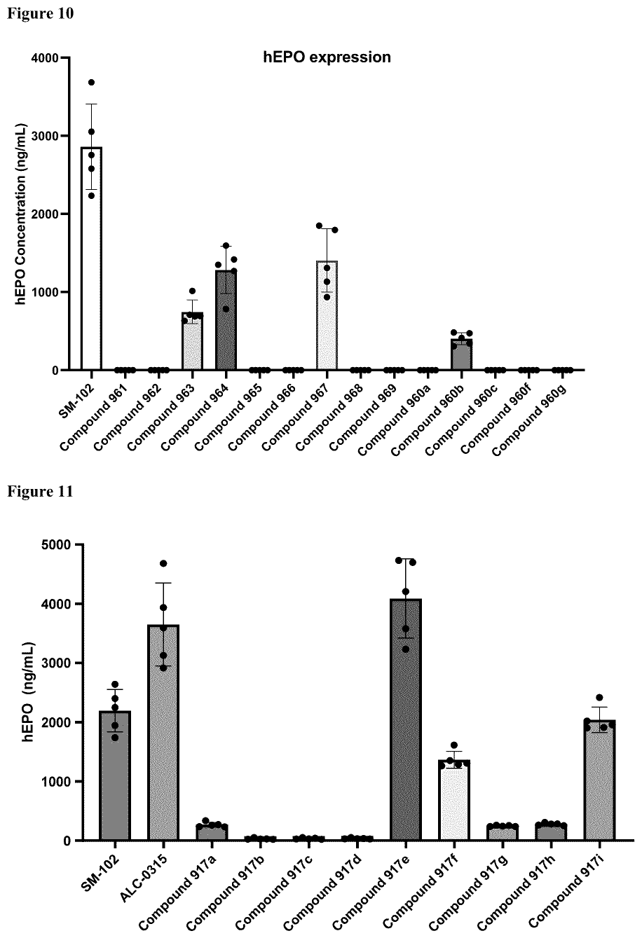

The in vivo studies were performed in C₅₇BL/6 female mice at 6 to 8 weeks weighing in at approximately 20 g. The LNPs formulated with different ionizable lipids at 0.2 mg/kg of hEPO mRNA were administered by tail vein injection and animals were euthanized at 6 h post-administration for blood serum sample collection. The hEPO levels from the samples were analyzed and cross-compared by enzyme-linked immunoassay (ELISA) according to manufacturer's protocol. The hEPO expression levels for each of the LNPs shown in Table 8 were compared to a control LNP comprising SM102 (DC Chemicals Cat. No. DC52025) and Dlin-MC3-DMA (BroadPharm Cat. No. BP-25497) (FIGS. 8 and 9). The hEPO expression levels for each of the LNPs shown in Table 9 were compared to a control LNP comprising SM102 (DC Chemicals Cat. No. DC52025) (FIG. 10). The hEPO expression levels for each of the LNPs shown in Table 10 were compared to a control LNP comprising SM-102 (DC Chemicals Cat. No. DC52025) and ALC-0315 (DC Chemicals Cat. No. DC42537) (FIG. 11).

### Example 15: In Vivo Expression Evaluation of Head Group Modification on Lipids in Lipid Nanoparticle Formulations

In this example, messenger RNA molecules encoding hEPO proteins were formulated in LNPs for delivery in vivo. The lipid nanoparticle formulations comprised of a lipid composition of a lipid:helper lipid:cholesterol:DMG-PEG2k at 50:10:38.5:1.5 mol %. The lipid mixture in ethanol was mixed with hEPO mRNA in RNA acidifying buffer (10 mM citrate, pH 4) at a N-to-P ratio of 6 using a microfluidic device (Precision NanoSystems, Inc.) at a combined flow rate of 10 mL/min (7.5 mL/min for aqueous buffer, RNA and 2.5 mL/min for ethanol, lipid mix). The resulting particles were neutralized by buffer exchange into 20 mM HEPES (pH 7.8), 50 mM sodium chloride, 15% (w/v) sucrose solution via PD-10 desalting column. The neutralized particles were concentrated using 100 kDa AMICON® Ultra centrifugal filters and sterile filtered using 0.2 um syringe filters. LNPs were then stored at −80C until use. Prior to in vivo dosing, LNPs were thawed at room temperature and diluted 2-fold with 20 mM HEPES (pH 7.8), resulting in a final dosing buffer of 20 mM HEPES (pH 7.8), 25 mM sodium chloride, 7.5% (w/v) sucrose. Resulting LNPs were characterized by dynamic light scattering (DLS) measurement for measuring its hydrodynamic diameter and polydispersity index (PDI) and the Zeta potential was measured using a Zetasizer (Malvern). Encapsulation efficiency (EE %) and total RNA concentration were quantitated using a fluorescence based Ribogreen assay, and the apparent pKa of the LNPs was measured by a fluorescence based TNS assay. The formulation and measurements for each of the LNPs are shown in Table 11 below.

The in vivo studies were performed in C57BL/6 female mice at 6 to 8 weeks weighing in at approximately 20 g. The LNPs formulated with different ionizable lipids at 0.2 mg/kg of hEPO mRNA were administered by tail vein injection and animals were euthanized at 6 h post-administration for blood serum sample collection. The hEPO levels from the samples were analyzed and cross-compared by enzyme-linked immunoassay (ELISA) according to manufacturer's protocol. The hEPO expression levels for each of the LNPs shown in Table 11 were compared to a control LNP comprising SM-102 (DC Chemicals Cat. No. DC52025) and ALC-0315 (DC Chemicals Cat. No. DC42537) (FIG. 17).

TABLE 11
Additional LNP formulations for in vivo studies

| | | | | | | Zeta | | |
|---|---|---|---|---|---|---|---|---|
| Ionizable | Lipid:DSPC:Cholesterol:DMG-PEG2k | | N:P | Size | | potential | Encapsulation | |
| Lipid | (mol %) | mRNA | ratio | (nm) | PDI | (mV) | Efficiency % | pKa |
| SM-102 | 50:10:38.5:1.5 | hEPO | 6 | 69.6 | 0.087 | −1.0 | 97.8 | 6.3 |
| ALC-0315 | 46.3:9.4:42.7:1.6 | hEPO | 6 | 74.7 | 0.280 | −7.5 | 91.0 | 6.0 |
| Compound 987a | 50:10:38.5:1.5 | hEPO | 6 | 73.2 | 0.104 | −3.5 | 95.7 | 6.4 |
| Compound 987b | 50:10:38.5:1.5 | hEPO | 6 | 74.1 | 0.102 | −2.1 | 93.6 | 6.3 |
| Compound 987c | 50:10:38.5:1.5 | hEPO | 6 | 72.0 | 0.066 | −6.6 | 98.0 | 5.9 |
| Compound 987d | 50:10:38.5:1.5 | hEPO | 6 | 62.9 | 0.092 | −1.5 | 99.2 | 5.7 |
| Compound 987e | 50:10:38.5:1.5 | hEPO | 6 | 70.5 | 0.110 | 0.8 | 99.4 | |
| Compound 987f | 50:10:38.5:1.5 | hEPO | 6 | 79.4 | 0.072 | −2.8 | 92.3 | 6.3 |
| Compound 987g | 50:10:38.5:1.5 | hEPO | 6 | 70.5 | 0.097 | −3.5 | 95.4 | 6.4 |
| Compound 987h | 50:10:38.5:1.5 | hEPO | 6 | 69.1 | 0.099 | 0.0 | 99.4 | 7.4 |
| Compound 987i | 50:10:38.5:1.5 | hEPO | 6 | 71.7 | 0.059 | −4.1 | 94.9 | 5.9 |
| Compound 987j | 50:10:38.5:1.5 | hEPO | 6 | 76.6 | 0.101 | −4.4 | 95.7 | 5.9 |
| Compound 987k | 50:10:38.5:1.5 | hEPO | 6 | 76.7 | 0.045 | −3.4 | 87.2 | 5.5 |
| Compound 987l | 50:10:38.5:1.5 | hEPO | 6 | 76.4 | 0.051 | −2.9 | 95.9 | 6.5 |
| Compound 987m | 50:10:38.5:1.5 | hEPO | 6 | 73.7 | 0.076 | −5.1 | 94.9 | 5.8 |
| Compound 987n | 50:10:38.5:1.5 | hEPO | 6 | 65.7 | 0.107 | −3.8 | 77.8 | 2.1 |
| Compound 987o | 50:10:38.5:1.5 | hEPO | 6 | 57.4 | 0.121 | −4.1 | 90.2 | 4.8 |
| Compound 987p | 50:10:38.5:1.5 | hEPO | 6 | 74.2 | 0.114 | −1.5 | 99.2 | 6.9 |

### Example 16: Storage Conditions and Stability Evaluation

In this example, LNPs encapsulating hEPO mRNA are formulated to evaluate long-term stability in different cold storage conditions including 2-5° C., −30° C., and −80° C. The lipid nanoparticle formulations comprise a lipid composition of lipid:helper lipid:cholesterol:DMG-PEG2k at 50:10:38.5:1.5 mol %. The lipid mixture in ethanol is mixed with hEPO mRNA in RNA acidifying buffer (10 mM citrate, pH 4) at a N-to-P ratio of 6 using a microfluidic device (Precision NanoSystems, Inc.) at a combined flow rate of 10 mL/min (7.5 mL/min for aqueous buffer, RNA and 2.5 mL/min for ethanol, lipid mix). The resulting particles are neutralized by buffer exchange into 20 mM tris buffer solution pH 7.4, via PD-10 desalting column. The neutralized particles are concentrated using 100 kDa AMICON® Ultra centrifugal filters, then diluted with a high sucrose content tris buffer to obtain an 8% sucrose containing LNP solution. The final solution is sterile filtered using 0.2 um syringe filters, then aliquoted appropriately. Resulting LNPs are characterized by dynamic light scattering (DLS) measurement for measuring its hydrodynamic radius and polydispersity index (PDI) and the Zeta potential is measured using a Zetasizer (Malvern). Encapsulation efficiency (EE %) and total RNA concentration are quantitated using a fluorescence based Ribogreen assay, and the apparent pKa of the LNPs is measured by a fluorescence based TNS assay.

Aliquoted vials are split into 3 groups and each group is placed into 2-5° C., −30° C., and −80° C. An aliquot from each group is pulled at week 1, 2, 3, 4 and every month thereafter for up to 6 months. The size and encapsulation efficiency are characterized for the different batches for stability assessment.

### Example 17: Scale-Up Process Compatibility

In this example, LNPs encapsulating hEPO mRNA are formulated at larger scale batches to evaluate scale-up process compatibility. The LNP formulations comprise a lipid composition of a lipid:helper lipid:cholesterol:DMG-PEG2k at 50:10:38.5:1.5 mol %. For small scale batches, the lipid mixture in ethanol is mixed with hEPO mRNA in RNA acidifying buffer (10 mM citrate, pH 4) at a N-to-P ratio of 6 using a microfluidic device (Precision NanoSystems, Inc.) at a combined flow rate of 10 mL/min (7.5 mL/min for aqueous buffer, RNA and 2.5 mL/min for ethanol, lipid mix). The resulting particles are neutralized by buffer exchange into Dulbecco's phosphate buffer solution via PD-10 desalting column. The neutralized particles are concentrated using 100 kDa AMICON® Ultra centrifugal filters and sterile filtered using 0.2 um syringe filters. For larger scale batches, the mixing is performed on a cross-T mixer and the mixed particles are neutralized then buffer exchanged into Dulbecco's phosphate buffer via tangential flow filtration. The concentrated samples are then sterile filtered by 0.2 um syringe filters. Resulting LNPs from the two batches are characterized by dynamic light scattering (DLS) measurement for measuring its hydrodynamic radius and polydispersity index (PDI) and the Zeta potential is measured using a Zetasizer (Malvern). Encapsulation efficiency (EE %) and total RNA concentration are quantitated using a fluorescence based Ribogreen assay, and the apparent pKa of the LNPs is measured by a fluorescence based TNS assay.

The in vivo studies are performed in C₅₇BL/6 female mice at 6 to 8 weeks weighing in at approximately 20 g. The LNPs formulated with different ionizable lipids at 2 mg/kg of hEPO mRNA are administered by tail vein injection and animals are euthanized at 6 h or 24 h post-administration for blood serum sample collection and liver tissue extraction. The hEPO levels from the samples were analyzed and cross-compared by enzyme-linked immunoassay (ELISA) according to manufacturer's protocol.

### Example 18: LNP Toxicity Study to Observe Cytokine Profiles and Aminotransferase activity

In this Example, Messenger RNA Molecules Encoding hEPO Proteins are formulated in LNPs for delivery in vivo. The lipid nanoparticle formulations comprise a lipid composition of lipid:helper lipid:cholesterol:DMG-PEG2k at 50:10:38.5:1.5 mol %. The lipid mixture in ethanol is mixed with hEPO mRNA in RNA acidifying buffer (10 mM citrate, pH 4) at a N-to-P ratio of 6 using a microfluidic device (Precision NanoSystems, Inc.) at a combined flow rate of 10 mL/min (7.5 mL/min for aqueous buffer, RNA and 2.5 mL/min for ethanol, lipid mix). The resulting particles are neutralized by buffer exchange into Dulbecco's phosphate buffer solution via PD-10 desalting column. The neutralized particles are concentrated using 100 kDa AMICON® Ultra centrifugal filters and sterile filtered using 0.2 um syringe filters. Resulting LNPs are characterized by dynamic light scattering (DLS) measurement for measuring its hydrodynamic radius and polydispersity index (PDI) and the Zeta potential is measured using a Zetasizer (Malvern). Encapsulation efficiency (EE %) and total RNA concentration are quantitated using a fluorescence based Ribogreen assay, and the apparent pKa of the LNPs is measured by a fluorescence based TNS assay.

The in vivo studies are performed in C₅₇BL/6 female mice at 6 to 8 weeks weighing in at approximately 20 g. The LNPs formulated with different ionizable lipids at 2 mg/kg of hEPO mRNA are administered by tail vein injection and animals are euthanized at 6 h or 24 h post-administration for blood serum sample collection and liver tissue extraction. The hEPO levels from the samples are analyzed and cross-compared by enzyme-linked immunoassay (ELISA) according to manufacturer's protocol.

For cytokine profiling of the tissue sample, the blood samples are processed and analyzed using a custom ProcartaPlex kit in a Luminex 200. The kit is customized to detect and quantitate IFN-alpha, IFN-beta, IFN-gamma, IL-1 beta, IL-6, IL-18, IL-22, IP-10 (CXCL10), MCP-1 (CCL2), MIP-2 alpha (CXCL2), and TNF alpha in mouse serum.

Alanine Aminotransferase (ALT)/Aspartate aminotransferase (AST) assay are performed on the blood serum samples according to manufacturer's instructions to assess liver damage with LNP treatment.

### Example 19: Vaccine

For Studies 1 and 2, detailed below, messenger RNA molecules encoding SARS-CoV-2 spike proteins (spike mRNA) were formulated in LNPs for delivery in vivo and antibody titers were assayed at 28 days after administration. The lipid nanoparticle formulations comprised a lipid composition of lipid:helper lipid:cholesterol:DMG-PEG2k in the ratios specified below. The lipid mixture in ethanol was mixed with spike mRNA in RNA acidifying buffer (10 mM citrate, pH 4) at a N-to-P ratio of 6 using a microfluidic device (Precision NanoSystems, Inc.) at a combined flow rate of 10 mL/min (7.5 mL/min for aqueous buffer, RNA and 2.5 mL/min for ethanol, lipid mix). The resulting particles were neutralized by buffer exchange into Dulbecco's phosphate buffer solution via PD-10 desalting column. The neutralized particles were concentrated using 100 kDa AMICON® Ultra centrifugal filters and sterile filtered using 0.2 um syringe filters. Resulting LNPs were characterized by dynamic light scattering (DLS) measurement for measuring their hydrodynamic radius and polydispersity index (PDI) and the Zeta potential was measured using a Zetasizer (Malvern). Encapsulation efficiency (EE %) and total RNA concentration were quantitated using a fluorescence based Ribogreen assay, and the apparent pKa of the LNPs was measured by a fluorescence based TNS assay.

The in vivo studies were performed in BALB-C female mice at 6 to 8 weeks weighing in at approximately 20 g. The spike mRNA were dosed at the indicated dosages by intramuscular injection, and a primary boost of the indicated dosage was administered after day 14 post-vaccination. Blood samples were collected at day 28 for an antibody titer assay.

### Study 1: Dosage and Formulation Study

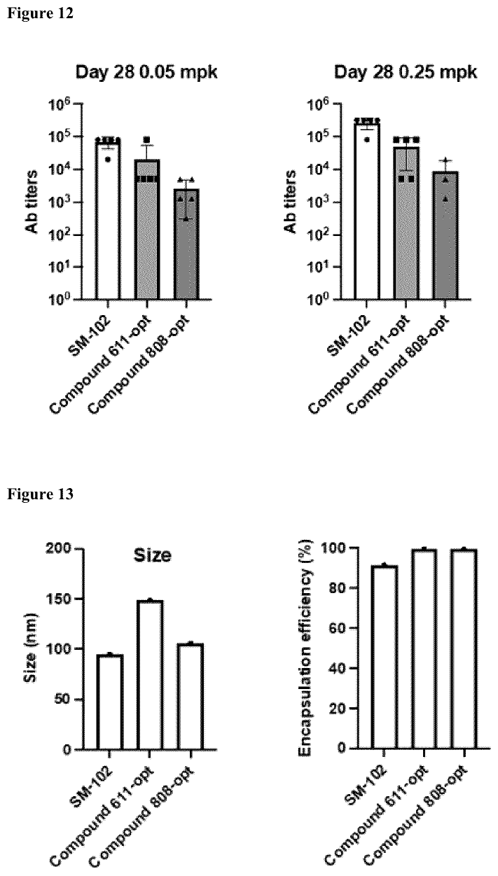

Following the procedure outlined above, Spike mRNA formulated in an LNP comprising an exemplary ionizable lipid were dosed by intramuscular injection at either 0.05 mg/kg or at 0.25 mg/kg at time (T)=0, and then boosted with either 0.05 mg/kg or at 0.25 mg/kg at T=14 days. The mice were bled and the antibody titers assayed at T=28 days. Results are shown in FIG. 12.

LNP formulation data for the LNPs used in Study 1 are shown below. All formulations use the standard mol. ratios of LNP components, unless noted otherwise by “opt.”

| | | Ionizable | | | |
|---|---|---|---|---|---|
| Formulation | Compound | Lipid | Chol | DSPC | PEG |
| Control | SM-102 | 50 | 38.5 | 10 | 1.5 |
| Compound 611-opt | 611 | 60 | 38.5 | 0 | 1.5 |
| Compound 808-opt | 808 | 30 | 58.5 | 10 | 1.5 |

Properties of the tested LNP formulations are shown below and in FIG. 13.

| Ionizable | Size | | |
|---|---|---|---|
| Lipid | (nm) | PDI | EE(%) |
| SM-102 | 94.8 | 0.065 | 91.5 |
| Compound 611-opt | 148.6 | 0.151 | 99.5 |
| Compound 808-opt | 105.6 | 0.042 | 99.4 |

### Study 2: Dosage and Formulation Study

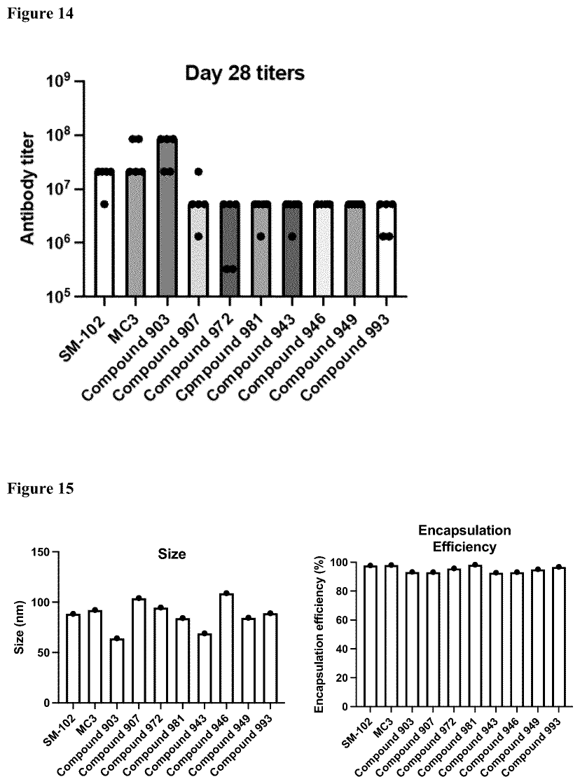

Spike mRNA formulated in an LNP comprising an exemplary ionizable lipid were dosed by intramuscular injection at 0.25 mg/kg at time (T)=0, and then boosted with 0.25 mg/kg at T=14 days. The mice were bled and the antibody titers assayed at T=28 days. Results are shown in FIG. 14.

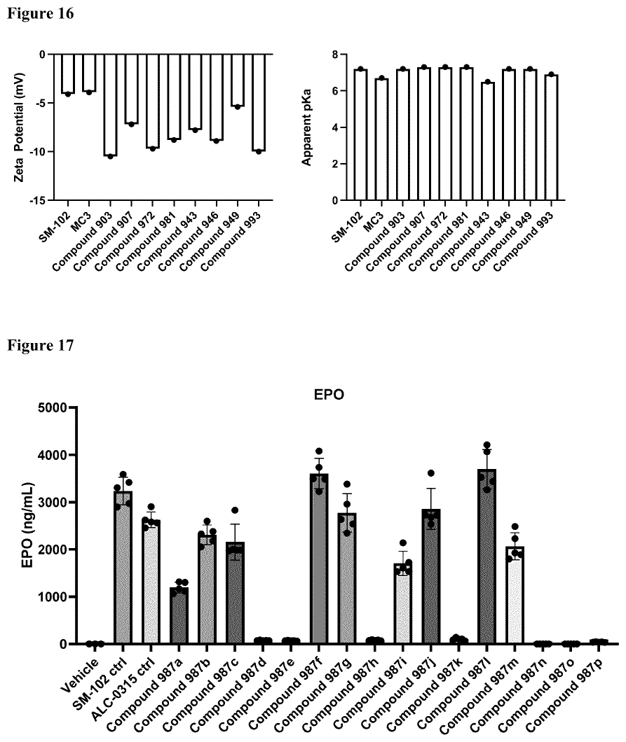

All formulations use the standard mol. ratios of LNP components, i.e., 50/10/38.5/1.5 mol % of ionizable lipid:helper lipid:cholesterol:DMG-PEG2k. Physical properties of these LNP formulations are shown below and in FIGS. 15-16.

| Ionizable | Size | | Zeta | EE | Apparent |
|---|---|---|---|---|---|
| Lipid | (nm) | PDI | (mV) | (%) | pKa |
| SM-102 | 88.4 | 0.127 | −4.1 | 97.8 | 7.2 |
| MC3 | 92.3 | 0.046 | −3.9 | 98 | 6.7 |
| Compound 903 | 64 | 0.049 | −10.5 | 93.3 | 7.2 |
| Compound 907 | 104.1 | 0.08 | −7.2 | 93.1 | 7.3 |
| Compound 972 | 94.7 | 0.093 | −9.7 | 95.8 | 7.3 |
| Compound 981 | 84.3 | 0.054 | −8.8 | 98.3 | 7.3 |
| Compound 943 | 69.1 | 0.051 | −7.8 | 92.8 | 6.5 |
| Compound 946 | 108.9 | 0.065 | −8.9 | 93.1 | 7.2 |
| Compound 949 | 84.5 | 0.093 | −5.4 | 95.1 | 7.2 |
| Compound 993 | 89 | 0.067 | −10 | 96.8 | 6.9 |

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

We claim:

1. A lipidoid having the structure of formula (X): 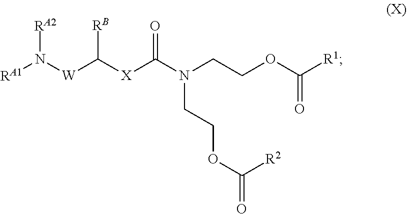
or a salt thereof;
wherein:
R^{A1} and R^{A2} are each independently H or alkyl;
or wherein R^{A1} and R^{A2}, taken together with the intervening nitrogen, form an optionally substituted heterocyclic ring;
R^{B} is H or alkyl;
X is a bond or —CH₂—;
W is a bond, —CH₂—, or —CH₂CH₂—; and
R¹ and R² are each independently branched (C₈-C₃₀)alkyl or linear (C₈-C₃₀)alkenyl; wherein a branched (C₈-C₃₀)alkyl may comprise more than one branch point, and a linear (C₈-C₃₀)alkenyl may comprise more than one carbon-carbon double bond.

2. The lipidoid of claim 1, wherein R^{A1} and R^{A2} are the same.

3. The lipidoid of claim 1, wherein R^{A1} and R^{A2} are different.

4. The lipidoid of claim 1, wherein R^{A1} and R^{A2} are each independently selected from H, methyl, ethyl, propyl, and isobutyl.

5. The lipidoid of claim 1, wherein R^{A1} and R^{A2}, taken together with the intervening nitrogen, form an optionally substituted 5-membered or 6-membered heterocyclic ring.

6. The lipidoid of claim 1, wherein R^{B} is alkyl.

7. The lipidoid of claim 1, wherein R^{B} is methyl.

8. The lipidoid of claim 1, wherein R¹ and R² are the same.

9. The lipidoid of claim 1, wherein R¹ and R² are different.

10. The lipidoid of claim 1, wherein R¹ and R² are each independently branched (C₁₂-C₂₀)alkyl.

11. The lipidoid of claim 1, wherein if R^{B} is H and R^{A1} and R^{A2} are both methyl, then R¹ and R² are each independently branched (C₈-C₃₀)alkyl

12. A lipidoid having the structure of formula (X): 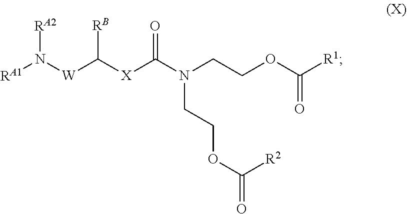
or a salt thereof;
wherein:
R^{A1} and R^{A2} are each alkyl;
or wherein R^{A1} and R^{A2}, taken together with the intervening nitrogen, form an optionally substituted heterocyclic ring;
R^{B} is H or alkyl;
X is a bond or —CH₂—;
W is a bond, —CH₂—, or —CH₂CH₂—; and
R¹ and R² are each independently branched (C₈-C₃₀)alkyl; wherein a branched (C₈-C₃₀)alkyl may comprise more than one branch point.

13. The lipidoid of claim 12, wherein R^{A1} and R^{A2} are the same.

14. The lipidoid of claim 12, wherein R^{A1} and R^{A2} are different.

15. The lipidoid of claim 12, wherein R^{A1} and R^{A2} are each independently selected from methyl, ethyl, propyl, and isobutyl.

16. The lipidoid of claim 12, wherein R^{A1} and R^{A2}, taken together with the intervening nitrogen, form an optionally substituted 5-membered or 6-membered heterocyclic ring.

17. The lipidoid of claim 12, wherein R^{B} is alkyl.

18. The lipidoid of claim 12, wherein R^{B} is methyl.

19. The lipidoid of claim 12, wherein R¹ and R² are the same.

20. The lipidoid of claim 12, wherein R¹ and R² are different.

21. The lipidoid of claim 12, wherein R¹ and R² are each independently branched (C₁₂-C₂₀)alkyl.

22. The lipidoid of claim 12, wherein when X is a bond, then W is —CH₂— or —CH₂CH₂—; and when W is a bond, then X is —CH₂—.

23. The lipidoid of claim 12, wherein when X is a bond, then W is —CH₂—; and when W is a bond, then X is —CH₂—.

24. A lipidoid having the structure of formula (I): 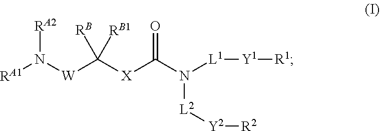
or a salt thereof;
wherein:
R^{A1} and R^{A2} are each independently H, alkyl, or hydroxyalkyl;
or wherein R^{A1} and R^{A2}, taken together with the intervening nitrogen, form an optionally substituted heterocyclic ring;
R^{B} and R^{B1} are each independently H, optionally substituted alkyl, alkenyl, cycloalkyl, arylalkyl, heteroarylalkyl, hydroxyalkyl, alkoxyalkyl, aminoalkyl, or thioalkyl;
or wherein R^{B} and R^{B1}, taken together with the intervening carbon, form an optionally substituted cycloalkyl or heterocyclic ring;
or R^{A1} and R^{B} taken together with the intervening N—CH atoms to which they are attached form an N-containing heterocyclic ring;
L¹ and L² are each independently —CH₂CH₂—, —(CH₂)₃—, —(CH₂)₄—, —CH(CH₃)CH₂—, or —CH₂CH(CH₃)—;
X is a bond, —CH₂—, or —CH₂CH₂—;
W is a bond, —CH₂—, or —CH₂CH₂—;
Y¹ and Y² are each independently —O(C═O)—, —S(C═O)—, or —O(C═O)O—; and
R¹ and R² are each independently linear (C₈-C₃₀)alkyl, branched (C₈-C₃₀)alkyl, linear (C₈-C₃₀)alkenyl, branched (C₈-C₃₀)alkenyl, linear (C₈-C₃₀)alkynyl, or branched (C₈-C₃₀)alkynyl; wherein a branched (C₈-C₃₀)alkyl, a branched (C₈-C₃₀)alkenyl, or a branched (C₈-C₃₀)alkynyl may comprise more than one branch point, and a linear or branched (C₈-C₃₀)alkenyl may comprise more than one carbon-carbon double bond, and a linear or branched (C₈-C₃₀)alkynyl may comprise more than one carbon-carbon triple bond;
provided that the compound is not:
L-Lysine-bis(O,O′-oleoyl-β-hydroxyethyl)amide;
L-Lysine-bis(O,O′-palmitoyl-β-hydroxyethyl)amide;
L-Lysine-bis(O,O′-myristoyl-β-hydroxyethyl)amide;
L-Ornithine-bis(O,O′-palmitoyl-β-hydroxyethyl)amide;
L-Ornithine-bis(O,O′-oleoyl-β-hydroxyethyl)amide;
L-Ornithine-bis(O,O′-myristoyl-β-hydroxyethyl)amide;
L-Arginine-bis(O,O′-palmitoyl-β-hydroxyethyl)amide;
L-Arginine-bis(O,O′-oleoyl-β-hydroxyethyl)amide;
L-Serine-bis(O,O′-oleoyl-β-hydroxyethyl)amide;
Glycine-bis(O,O′-palmitoyl-β-hydroxyethyl)amide;
Sarcosine-bis(O,O′-palmitoyl-β-hydroxyethyl)amide;
L-Histidine-bis(O,O′-palmitoyl-β-hydroxyethyl)amide;
L-Glutamine-bis(O,O′-palmitoyl-β-hydroxyethyl)amide;
L-Glutamic acid-γ-N′-bis(O,O′-palmitoyl-β-hydroxyethyl)amide;
L-aspartic acid-β-N′-bis(O,O′-palmitoyl-β-hydroxyethyl)amide; or
L-aspartic acid-α-N′-bis(O,O′-palmitoyl-β-hydroxyethyl)amide.

25. The lipidoid of claim 24, wherein if R^{B} and R^{B1} are both H and R^{A1} and R^{A2} are both methyl, then R¹ and R² are each independently branched (C₈-C₃₀)alkyl.

26. The lipidoid of claim 24, wherein 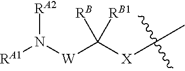
is represented by

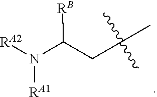

27. The lipidoid of claim 24, wherein 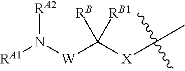
is represented by

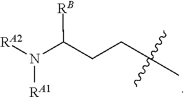

28. The lipidoid of claim 24, wherein 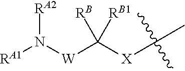
is represented by

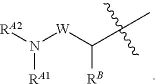

29. The lipidoid of claim 24, wherein R^{A1} and R^{A2} are the same.

30. The lipidoid of claim 24, wherein R^{A1} and R^{A2} are different.

31. The lipidoid of claim 24, wherein R^{A1} and R^{A2} are each independently selected from methyl, ethyl, propyl, and isobutyl.

32. The lipidoid of claim 24, wherein R^{A1} and R^{A2}, taken together with the intervening nitrogen, form an optionally substituted 5-membered or 6-membered heterocyclic ring.

33. A lipidoid having the structure of formula (II): 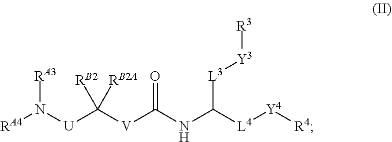
or a salt thereof;
wherein:
R^{A3} and R^{A4} are each independently H, alkyl, or hydroxyalkyl;
or wherein R^{A3} and R^{A4}, taken together with the intervening nitrogen, form an optionally substituted heterocycloalkyl ring;
R^{B2} and R^{B2A} are each independently H, optionally substituted alkyl, alkenyl, cycloalkyl, arylalkyl, heteroarylalkyl, hydroxyalkyl, alkoxyalkyl, aminoalkyl, or thioalkyl;
or wherein R^{B2} and R^{B2A}, taken together with the intervening carbon, form an optionally substituted cycloalkyl or heterocycloalkyl ring;
or R^{A3} and R^{B2} taken together with the intervening N—CH atoms to which they are attached form an N-containing heterocyclic ring;
L³ and L⁴ are each independently —CH₂—, —CH₂CH₂—, —(CH₂)₃—, —(CH₂)₄—, —CH(CH₃)CH₂—, or —CH₂CH(CH₃)—;
V is a bond, —CH₂— or —CH₂CH₂—;
U is a bond, —CH₂— or —CH₂CH₂—;
Y³ and Y⁴ are each independently —O(C═O)—, —S(C═O)—, or —O(C═O)O—; and
R³ and R⁴ are each independently linear (C₈-C₃₀)alkyl, branched (C₈-C₃₀)alkyl, linear (C₈-C₃₀)alkenyl, branched (C₈-C₃₀)alkenyl, linear (C₈-C₃₀)alkynyl, or branched (C₈-C₃₀)alkynyl; wherein a branched (C₈-C₃₀)alkyl, a branched (C₈-C₃₀)alkenyl, or a branched (C₈-C₃₀)alkynyl may comprise more than one branch point, and a linear or branched (C₈-C₃₀)alkenyl may comprise more than one carbon-carbon double bond, and a linear or branched (C₈-C₃₀)alkynyl may comprise more than one carbon-carbon triple bond.

34. A nanoparticle composition, comprising a lipidoid of claim 1, or a pharmaceutically acceptable salt thereof.

35. The nanoparticle composition of claim 34, further comprising a PEGylated lipid, a sterol, a phospholipid, and/or a neutral lipid.

36. The nanoparticle composition of claim 35, wherein the nanoparticle composition is substantially free of a phospholipid.

37. The nanoparticle composition of claim 34, further comprising a therapeutic agent.

38. The nanoparticle composition of claim 34, further comprising an antigen; wherein the antigen is a protein or a nucleic acid; the antigen is a protein; or the antigen is a nucleic acid.

39. The nanoparticle composition of claim 34, further comprising an mRNA molecule that encodes an antigen.

40. A method of delivering a therapeutic agent, comprising administering to a subject in need thereof an effective amount of the nanoparticle composition of claim 37.

41. A method of vaccination, comprising administering to a subject in need thereof an effective amount of the nanoparticle composition of claim 39.
